(19) United States
(12) Patent Application Publication
(10) Pub. No.: US 2019/0382477 A1
(43) Pub. Date: Dec. 19, 2019

(54) ANTI-PVRIG/ANTI-TIGIT BISPECIFIC ANTIBODIES AND METHODS OF USE

(71) Applicant: Compugen Ltd., Holon (IL)

(72) Inventors: Andrew W. Drake, South San Francisco, CA (US); Sandeep Kumar, South San Francisco, CA (US); Sayantan Mitra, South San Francisco, CA (US); Adam Sallers, South San Francisco, CA (US); Sarah Whelan, South San Francisco, CA (US); Arun Kashyap, South San Francisco, CA (US); Keith Akama, South San Francisco, CA (US); Neha Yevaekar, South San Francisco, CA (US)

(21) Appl. No.: 16/428,856

(22) Filed: May 31, 2019

Related U.S. Application Data
(60) Provisional application No. 62/679,703, filed on Jun. 1, 2018.
(60) Provisional application No. 62/773,586, filed on Nov. 30, 2018.

(51) Int. Cl. C07K 16/28; C07K 16/46

(52) U.S. Cl. CPC C07K 16/28; C07K 16/468; C07K 2317/92; C07K 2317/24; C07K 2317/31; C07K 16/2818

(57) ABSTRACT

Anti-PVRIG, anti-TIGIT, and anti-PVRIG/anti-TIGIT bispecific antibodies are provided, as well as compositions, and methods of using the antibodies for the treatment of cancer.

### CROSS-REFERENCE TO RELATED APPLICATIONS

This application claims priority to U.S. Provisional Patent Applications Nos. 62/679,703, filed Jun. 1, 2018 and 62/773,586, filed Nov. 30, 2018, all of which are incorporated by reference in their entireties.

The instant application contains a Sequence Listing which has been submitted electronically in ASCII format and is hereby incorporated by reference in its entirety. The ASCII copy, created on May 29, 2019, is named 114386-5011-WO_SL.txt and is 2,631,244 bytes in size.

### I. BACKGROUND OF THE INVENTION

Naïve T cells must receive two independent signals from antigen-presenting cells (APC) in order to become productively activated. The first, Signal 1, is antigen-specific and occurs when T cell antigen receptors encounter the appropriate antigen-MHC complex on the APC. The fate of the immune response is determined by a second, antigen-independent signal (Signal 2) which is delivered through a T cell costimulatory molecule that engages its APC-expressed ligand. This second signal could be either stimulatory (positive costimulation) or inhibitory (negative costimulation or coinhibition). In the absence of a costimulatory signal, or in the presence of a coinhibitory signal, T-cell activation is impaired or aborted, which may lead to a state of antigen-specific unresponsiveness (known as T-cell anergy), or may result in T-cell apoptotic death.

Costimulatory molecule pairs usually consist of ligands expressed on APCs and their cognate receptors expressed on T cells. The prototype ligand/receptor pairs of costimulatory molecules are B7/CD28 and CD40/CD40L. The B7 family consists of structurally related, cell-surface protein ligands, which may provide stimulatory or inhibitory input to an immune response. Members of the B7 family are structurally related, with the extracellular domain containing at least one variable or constant immunoglobulin domain.

Both positive and negative costimulatory signals play critical roles in the regulation of cell-mediated immune responses, and molecules that mediate these signals have proven to be effective targets for immunomodulation. Based on this knowledge, several therapeutic approaches that involve targeting of costimulatory molecules have been developed, and were shown to be useful for prevention and treatment of cancer by turning on, or preventing the turning off, of immune responses in cancer patients and for prevention and treatment of autoimmune diseases and inflammatory diseases, as well as rejection of allogenic transplantation, each by turning off uncontrolled immune responses, or by induction of “off signal” by negative costimulation (or coinhibition) in subjects with these pathological conditions.

Manipulation of the signals delivered by B7 ligands has shown potential in the treatment of autoimmunity, inflammatory diseases, and transplant rejection. Therapeutic strategies include blocking of costimulation using monoclonal antibodies to the ligand or to the receptor of a costimulatory pair, or using soluble fusion proteins composed of the costimulatory receptor that may bind and block its appropriate ligand. Another approach is induction of co-inhibition using soluble fusion protein of an inhibitory ligand. These approaches rely, at least partially, on the eventual deletion of auto- or allo-reactive T cells (which are responsible for the pathogenic processes in autoimmune diseases or transplantation, respectively), presumably because in the absence of costimulation (which induces cell survival genes) T cells become highly susceptible to induction of apoptosis. Thus, novel agents that are capable of modulating costimulatory signals, without compromising the immune system's ability to defend against pathogens, are highly advantageous for treatment and prevention of such pathological conditions.

Costimulatory pathways play an important role in tumor development. Interestingly, tumors have been shown to evade immune destruction by impeding T cell activation through inhibition of co-stimulatory factors in the B7-CD28 and TNF families, as well as by attracting regulatory T cells, which inhibit anti-tumor T cell responses (see Wang (2006), “Immune Suppression by Tumor Specific CD4⁺ Regulatory T cells in Cancer”, Semin. Cancer. Biol. 16:73-79; Greenwald, et al. (2005), “The B7 Family Revisited”, Ann. Rev. Immunol. 23:515-48; Watts (2005), “TNF/TNFR Family Members in Co-stimulation of T Cell Responses”, Ann. Rev. Immunol. 23:23-68; Sadum, et al., (2007) “Immune Signatures of Murine and Human Cancers Reveal Unique Mechanisms of Tumor Escape and New Targets for Cancer Immunotherapy”, Clin. Canc. Res. 13(13): 4016-4025). Such tumor expressed co-stimulatory molecules have become attractive cancer biomarkers and may serve as tumor-associated antigens (TAAs). Furthermore, costimulatory pathways have been identified as immunologic checkpoints that attenuate T cell dependent immune responses, both at the level of initiation and effector function within tumor metastases.

Over the past decade, agonists and/or antagonists to various costimulatory proteins have been developed for treating autoimmune diseases, graft rejection, allergy and cancer. For example, CTLA4-Ig (Abatacept, Orencia®) is approved for treatment of RA, mutated CTLA4-Ig (Belatacept, Nulojix®) for prevention of acute kidney transplant rejection and by the anti-CTLA4 antibody (Ipilimumab, Yervoy®), recently approved for the treatment of melanoma. Other costimulation regulators have been approved, such as the anti-PD-1 antibodies of Merck (Keytruda®) and BMS (Opdivo®), have been approved for cancer treatments and are in testing for viral infections as well.

However, while monotherapy with anti-checkpoint inhibitor antibodies have shown promise, a number of studies (Ahmadzadeh et al., Blood 114:1537 (2009), Matsuzaki et al., PNAS 107(17):7875-7880 (2010), Fourcade et al., Cancer Res. 72(4):887-896 (2012) and Gros et al., J. Clinical Invest. 124(5):2246 (2014)) examining tumor-infiltrating lymphocytes (TILs) have shown that TILs commonly express multiple checkpoint receptors. Moreover, it is likely that TILs that express multiple checkpoints are in fact the most tumor-reactive. In contrast, non-tumor reactive T cells in the periphery are more likely to express a single checkpoint. Checkpoint blockade with monospecific full-length antibodies is likely nondiscriminatory with regards to de-repression of tumor-reactive TILs versus autoantigen-reactive single expressing T cells that are assumed to contribute to autoimmune toxicities.

One target of interest is PVRIG. PVRIG, also called Poliovirus Receptor Related Immunoglobulin Domain Containing Protein, Q6DKI7 or C7orf15, is a transmembrane domain protein of 326 amino acids in length, with a signal peptide (spanning from amino acid 1 to 40), an extracellular domain (spanning from amino acid 41 to 171), a transmembrane domain (spanning from amino acid 172 to 190) and a cytoplasmic domain (spanning from amino acid 191 to 326). PVRIG binds to Poliovirus receptor-related 2 protein (PVLR2, also known as nectin-2, CD112 or herpesvirus entry mediator B, (HVEB) a human plasma membrane glycoprotein), the binding partner of PVRIG.

Another target of interest is TIGIT. TIGIT is a coinhibitory receptor that is highly expressed on effector & regulatory (Treg) CD4+ T cells, effector CD8+ T cells, and NK cells. TIGIT has been shown to attenuate immune response by (1) direct signaling, (2) inducing ligand signaling, and (3) competition with and disruption of signaling by the costimulatory receptor CD226 (also known as DNAM-1). TIGIT signaling has been the most well-studied in NK cells, where it has been demonstrated that engagement with its cognate ligand, poliovirus receptor (PVR, also known as CD155) directly suppresses NK cell cytotoxicity through its cytoplasmic ITIM domain. Knockout of the TIGIT gene or antibody blockade of the TIGIT/PVR interaction has shown to enhance NK cell killing in vitro, as well as to exacerbate autoimmune diseases in vivo. In addition to its direct effects on T- and NK cells, TIGIT can induce PVR-mediated signaling in dendritic or tumor cells, leading to the increase in production of anti-inflammatory cytokines such as IL10. In T-cells TIGIT can also inhibit lymphocyte responses by disrupting homodimerization of the costimulatory receptor CD226, and by competing with it for binding to PVR.

TIGIT is highly expressed on lymphocytes, including Tumor Infiltrating Lymphocytes (TILs) and Tregs, that infiltrate different types of tumors. PVR is also broadly expressed in tumors, suggesting that the TIGIT-PVR signaling axis may be a dominant immune escape mechanism for cancer. Notably, TIGIT expression is tightly correlated with the expression of another important coinhibitory receptor, PD1. TIGIT and PD1 are co-expressed on the TILs of numerous human and murine tumors. Unlike TIGIT and CTLA4, PD1 inhibition of T cell responses does not involve competition for ligand binding with a costimulatory receptor.

Accordingly, PVRIG/TIGIT bispecific antibodies, capable of targeting both pathways, are an attractive target for single antibody therapy. Such antibodies will allow for targeting of multiple checkpoint receptors and provide therapeutic importance in the treatment of cancer. Also provided are anti-PVRIG and anti-TIGIT antibodies for use as described herein.

### II. BRIEF SUMMARY OF THE INVENTION

Accordingly, the present invention provides an anti-PVRIG/anti-TIGIT bispecific antibody that monovalently binds a human PVRIG and monovalently binds TIGIT for use in activating T cells for the treatment of cancer.

In some embodiments, the present invention provides an anti-PVRIG/anti-TIGIT bispecific antibody that monovalently binds a human PVRIG and monovalently binds TIGIT for use in activating T cells and/or NK cells for the treatment of cancer.

In some embodiments, the anti-PVRIG/anti-TIGIT bispecific antibody comprises:
a) a first antigen binding portion comprising:
   i. a first heavy chain variable domain comprising a vhCDR1, vhCDR2, and vhCDR3 from an anti-PVRIG antibody; and
   ii. a first light chain variable domain comprising a vlCDR1, vlCDR2, and vlCDR3 from an anti-PVRIG antibody;
      wherein the anti-PVRIG antibody is selected from the group consisting of CHA.7.518.4, CHA.7.518.1, CHA.7.518, CHA.7.524 CHA.7.530, CHA.7.538_1, CHA.7.538_2, CHA.7.502, CHA.7.503, CHA.7.506, CHA.7.508, CHA.7.510, CHA.7.512, CHA.7.514, CHA.7.516, CHA.7.518, CHA.7.520.1, CHA.7.520.2, CHA.7.522, CHA.7.524, CHA.7.526, CHA.7.527, CHA.7.528, CHA.7.530, CHA.7.534, CHA.7.535, CHA.7.537, CHA.7.538.1, CHA.7.538.2, CHA.7.543, CHA.7.544, CHA.7.545, CHA.7.546, CHA.7.547, CHA.7.548, CHA.7.549,CHA.7.550, CHA7.538.1.2, CPA.7.021, CPA.7.001, CPA.7.003, CPA.7.004, CPA.7.006, CPA.7.008, CPA.7.009, CPA.7.010, CPA.7.011, CPA.7.012, CPA.7.013, CPA.7.014, CPA.7.015, CPA.7.017, CPA.7.018, CPA.7.019,CPA.7.022, CPA.7.023, CPA.7.024, CPA.7.033, CPA.7.034, CPA.7.036, CPA.7.040, CPA.7.046, CPA.7.047, CPA.7.049, CPA.7.050, and CHA.7.518; and
a) a second antigen binding portion comprising an anti-TIGIT antigen binding domain.

In some embodiments, the anti-PVRIG/anti-TIGIT bispecific antibody comprises:
a) a first antigen binding portion comprising an anti-PVRIG antigen binding domain; and
b) a second antigen binding portion comprising:
   i. a second heavy chain variable domain comprising a vhCDR1, vhCDR2, and vhCDR3 from an anti-TIGIT antibody; and
   ii. a second light chain variable domain comprising a vlCDR1, vlCDR2, and vlCDR3 from an anti-TIGIT antibody;
      wherein the anti-TIGIT antibody is selected from the group consisting of CPA.9.086, CHA.9.547.18, CPA.9.018, CPA.9.027, CPA.9.049, CPA.9.057, CPA.9.059, CPA.9.083, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, and CHA.9.541.8.

In some embodiments, the anti-PVRIG/anti-TIGIT bispecific antibody comprises:
a) a first antigen binding portion comprising:
   i. a first heavy chain variable domain comprising a vhCDR1, vhCDR2, and vhCDR3 from an anti-PVRIG antibody; and
   ii. a first light chain variable domain comprising a vlCDR1, vlCDR2, and vlCDR3 from an anti-PVRIG antibody
      wherein the anti-PVRIG antibody is selected from the group consisting of CHA.7.518.4, CHA.7.518.1, humanized CHA.7.518, humanized CHA.7.524, humanized CHA.7.530, humanized CHA.7.538_1, humanized CHA.7.538_2, CHA.7.502, CHA.7.503, CHA.7.506, CHA.7.508, CHA.7.510, CHA.7.512, CHA.7.514, CHA.7.516, CHA.7.518, CHA.7.520.1, CHA.7.520.2, CHA.7.522, CHA.7.524, CHA.7.526, CHA.7.527, CHA.7.528, CHA.7.530, CHA.7.534, CHA.7.535, CHA.7.537, CHA.7.538.1, CHA.7.538.2, CHA.7.543, CHA.7.544, CHA.7.545, CHA.7.546, CHA.7.547, CHA.7.548, CHA.7.549,CHA.7.550, CHA7.538.1.2, CPA.7.021, CPA.7.001, CPA.7.003, CPA.7.004, CPA.7.006, CPA.7.008, CPA.7.009, CPA.7.010, CPA.7.011, CPA.7.012, CPA.7.013, CPA.7.014, CPA.7.015, CPA.7.017, CPA.7.018, CPA.7.019,CPA.7.022, CPA.7.023, CPA.7.024, CPA.7.033, CPA.7.034, CPA.7.036, CPA.7.040, CPA.7.046, CPA.7.047, CPA.7.049, and CPA.7.050CHA.7.518; and
b) a second antigen binding portion comprising:
   i. a second heavy chain variable domain comprising a vhCDR1, vhCDR2, and vhCDR3 from an anti-TIGIT antibody; and
   ii. a second light chain variable domain comprising a vlCDR1, vlCDR2, and vlCDR3 from an anti-TIGIT antibody;
      wherein the anti-TIGIT antibody is selected from the group consisting of CPA.9.086, CHA.9.547.18, CPA.9.018, CPA.9.027, CPA.9.049, CPA.9.057, CPA.9.059, CPA.9.083, CPA.9.086, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, and CHA.9.541.8.

In some embodiments, the first antigen binding portion comprises:
i. a first heavy chain comprising VH-CH1-hinge-CH2-CH3; and
ii. a first light chain comprising VL-CL, wherein the CL is the constant domain of either a kappa or lambda antibody.

In some embodiments, the first heavy chain CH3 comprises the amino acid substitutions S354C, E356D, M358L, and T366W.

In some embodiments, the CL is kappa.

In some embodiments, the second antigen binding portion comprises:
i. a second heavy chain comprising HC-CL-hinge-CH2-CH3, wherein the CL is either kappa or lambda; and
ii. a second light chain comprising VL-CH1.

In some embodiments, the second heavy chain CH3 comprises the amino acid substitutions Y349C, E356D, M358L, T366S, L368A, and Y407V.

In some embodiments, the CL is lambda.

In some embodiments, the CL is kappa.

In some embodiments, the anti-PVRIG/anti-TIGIT bispecific antibody comprises:
a) a first antigen binding portion comprising an anti-PVRIG antigen binding domain; and
b) a second anti-TIGIT antigen binding portion comprising:

i.
a second heavy chain variable region
comprising CPA.9.086 VH
(EVQLVETGGGLIQPGRSLRLSCAASGFTFSSYAMHWVRQAPG
KGLEWVAVISYAGEVKYYADSVKGRFTISRDNSKNTLYLQMN
SLRAEDTAVYYCARDPLPLHYYGMDVWGQGTTVTVSS; SEQ
ID NO: 1634);
and
ii.
a second light chain variable region
comprising CPA.9.086 VL
(QSALTQPRSASGNPGQRVTISCSGSSSNMGRRPVNWYQQIPGT
APKLLIYSQNQRPSGVPDRFSGSQSGTSASLTISGLQSEDEAEYF
CAVWDDIGRVLQLGGGTQLAVL; SEQ ID NO: 1639).

In some embodiments, the anti-PVRIG/anti-TIGIT bispecific antibody comprises:
c) a first antigen binding portion comprising an anti-PVRIG antigen binding domain; and
d) a second anti-TIGIT antigen binding portion comprising:

i.
a second heavy chain variable region
comprising CPA.9.547.18 VH
(EVQLVESGGGLVQPGGSLRLSCAASGFTFSSYIMSWVRQAPGK
GLEWVATISGGSKGQYYADSVKGRFTISRDNSKNTLYLQMNSL
RAEDTAVYYCAKWLLSYYAMDYWGQGTLVTVSS; SEQ ID
NO: 1664);
and
ii.
a second light chain variable region
comprising CPA.9.547.18 VL
(DIQMTQSPSSLSASVGDRITITCRASQSMAIWLSWYQQKPGKA
PKLLIYKASKSHTGVPSRFSGSGSGTDFTLTISSLQPEDFATYYC
QQGQSYPYTFGQGTKLEIK; SEQ ID NO: 1668).

In some embodiments, the anti-PVRIG/anti-TIGIT bispecific antibody comprises:
a) a first antigen binding portion comprising an anti-PVRIG binding doming comprising:

i.
a first heavy chain variable region
comprising CHA.7.518.1 VH
(QVQLVQSGAEVKKPGASVKVSCKASGYTFTDYNINWVRQAP
GQGLEWMGYIYPYIGGSGYAQKFQGRVTMTRDTSTSTVYMEL
SSLRSEDTAVYYCAREDKTARNAMDYWGQGTLVTVSS; SEQ
ID NO: 1539);
and
ii.
a first light chain comprising CHA.7.518.1 VL
(DIQMTQSPSSLSASVGDRVTITCRVSENIYSNLAWYQQKPGKA
PKLLIYEATNLAEGVPSRFSGSGSGTDFTLTISSLQPEDFATYYC
QHFWGTPYTFGQGTKLEIK; SEQ ID NO: 1544);

and
b) a second antigen binding portion comprising an anti-TIGIT antigen binding domain.

In some embodiments, the anti-PVRIG/anti-TIGIT bispecific antibody comprises:
a) a first antigen binding portion comprising an anti-PVRIG binding doming comprising:

i.
a first heavy chain variable region
comprising CHA.7.518.4 VH
(QVQLVQSGAEVKKPGASVKVSCKASGYTFTDYNINWVRQAP
GQGLEWMGYIYPYIGGSGYAQKFQGRVTMTRDTSTSTVYMEL
SSLRSEDTAVYYCAREDKTARNAMDYWGQGTLVTVSS; SEQ
ID NO: 3179);
and
ii.
a first light chain comprising CHA.7.518.4 VL
(DIQMTQSPSSLSASVGDRVTITCRVSENIYDVLAWYQQKPGKA
PKLLIYEATNLAEGVPSRFSGSGSGTDFTLTISSLQPEDFATYYC
QHFWGTPYTFGQGTKLEIK; SEQ ID NO: 3180);

and
b) a second antigen binding portion comprising an anti-TIGIT antigen binding domain.

In some embodiments, the anti-PVRIG/anti-TIGIT bispecific antibody comprises:
a) a first antigen binding portion comprising an anti-PVRIG binding doming comprising:

i.
a first heavy chain variable region
comprising CHA.7.518.1 VH
(QVQLVQSGAEVKKPGASVKVSCKASGYTFTDYNINWVRQAP
GQGLEWMGYIYPYIGGSGYAQKFQGRVTMTRDTSTSTVYMEL
SSLRSEDTAVYYCAREDKTARNAMDYWGQGTLVTVSS; SEQ
ID NO: 1539);
and
ii.
a first light chain variable region
comprising CHA.7.518.1
(DIQMTQSPSSLSASVGDRVTITCRVSENIYSNLAWYQQKPGKA
PKLLIYEATNLAEGVPSRFSGSGSGTDFTLTISSLQPEDFATYYC
QHFWGTPYTFGQGTKLEIK; SEQ ID NO: 1544):

and
b) a second antigen binding portion comprising an anti-TIGIT binding doming comprising

i.
a first heavy chain variable region
comprising CPA.9.086 VH
(EVQLVETGGGLIQPGRSLRLSCAASGFTFSSYAMHWVRQAPG
KGLEWVAVISYAGEVKYYADSVKGRFTISRDNSKNTLYLQMN
SLRAEDTAVYYCARDPLPLHYYGMDVWGQGTTVTVSS; SEQ
ID NO: 1634);
and
ii.
a first light chain variable region
comprising CPA.9.086 VL
(QSALTQPRSASGNPGQRVTISCSGSSSNMGRRPVNWYQQIPGT
APKLLIYSQNQRPSGVPDRFSGSQSGTSASLTISGLQSEDEAEYF
CAVWDDIGRVLQLGGGTQLAVL; SEQ ID NO: 1639).

In some embodiments, the anti-PVRIG/anti-TIGIT bispecific antibody comprises:
a) a first antigen binding portion comprising an anti-PVRIG binding doming comprising:

i.
a first heavy chain variable region
comprising CHA.7.518.4 VH
(QVQLVQSGAEVKKPGASVKVSCKASGYTFTDYNINWVRQAP
GQGLEWMGYIYPYIGGSGYAQKFQGRVTMTRDTSTSTVYMEL
SSLRSEDTAVYYCAREDKTARNAMDYWGQGTLVTVSS; SEQ
ID NO: 3179);
and
ii.
a first light chain comprising CHA.7.518.4 VL
(DIQMTQSPSSLSASVGDRVTITCRVSENIYDVLAWYQQKPGKA
PKLLIYEATNLAEGVPSRFSGSGSGTDFTLTISSLQPEDFATYYC
QHFWGTPYTFGQGTKLEIK; SEQ ID NO: 3180)

and
b) a second antigen binding portion comprising an anti-TIGIT binding doming comprising:

i.
a first heavy chain variable region
comprising CPA.9.086 VH
(EVQLVETGGGLIQPGRSLRLSCAASGFTFSSYAMHWVRQAPG
KGLEWVAVISYAGEVKYYADSVKGRFTISRDNSKNTLYLQMN
SLRAEDTAVYYCARDPLPLHYYGMDVWGQGTTVTVSS; SEQ
ID NO: 1634);
and
ii.
a first light chain variable region
comprising CPA.9.086 VL
(QSALTQPRSASGNPGQRVTISCSGSSSNMGRRPVNWYQQIPGT
APKLLIYSQNQRPSGVPDRFSGSQSGTSASLTISGLQSEDEAEYF
CAVWDDIGRVLQLGGGTQLAVL; SEQ ID NO: 1639).

In some embodiments, the anti-PVRIG/anti-TIGIT bispecific antibody comprises:
a) a first antigen binding portion comprising an anti-PVRIG binding doming comprising:

i. a first heavy chain variable region comprising
CHA.7.518.1
(QVQLVQSGAEVKKPGASVKVSCKASGYTFTDYNINWVRQAP
GQGLEWMGYIYPYIGGSGYAQKFQGRVTMTRDTSTSTVYMEL
SSLRSEDTAVYYCAREDKTARNAMDYWGQGTLVTVSS; SEQ
ID NO: 1539);
and
ii. a first light chain variable region comprising
CHA.7.518.1 VL
(DIQMTQSPSSLSASVGDRVTITCRVSENIYSNLAWYQQKPGKA
PKLLIYEATNLAEGVPSRFSGSGSGTDFTLTISSLQPEDFATYYC
QHFWGTPYTFGQGTKLEIK; SEQ ID NO: 1544);

and
b) a second antigen binding portion comprising an anti-TIGIT binding doming comprising:

i. a first heavy chain variable region comprising
CHA.9.547.18 HC
(EVQLVESGGGLVQPGGSLRLSCAASGFTFSSYIMSWVRQAPGK
GLEWVATISGGSKGQYYADSVKGRFTISRDNSKNTLYLQMNSL
RAEDTAVYYCAKWLLSYYAMDYWGQGTLVTVSS; SEQ ID
NO: 1664);
and
ii. a first light chain variable region comprising
CHA.9.547.18 VL
(DIQMTQSPSSLSASVGDRITITCRASQSMAIWLSWYQQKPGKA
PKLLIYKASKSHTGVPSRFSGSGSGTDFTLTISSLQPEDFATYYC
QQGQSYPYTFGQGTKLEIK; SEQ ID NO: 1668).

In some embodiments, the anti-PVRIG/anti-TIGIT bispecific antibody comprises:
a) a first antigen binding portion comprising an anti-PVRIG binding doming comprising:

i. a first heavy chain variable region comprising
CHA.7.518.4 VH
(QVQLVQSGAEVKKPGASVKVSCKASGYTFTDYNINWVRQAP
GQGLEWMGYIYPYIGGSGYAQKFQGRVTMTRDTSTSTVYMEL
SSLRSEDTAVYYCAREDKTARNAMDYWGQGTLVTVSS; SEQ
ID NO: 3179);
and
ii. a first light chain comprising CHA.7.518.4 VL
(DIQMTQSPSSLSASVGDRVTITCRVSENIYDVLAWYQQKPGKA
PKLLIYEATNLAEGVPSRFSGSGSGTDFTLTISSLQPEDFATYYC
QHFWGTPYTFGQGTKLEIK; SEQ ID NO: 3180);

and
b) a second antigen binding portion comprising an anti-TIGIT binding doming comprising:

i. a first heavy chain variable region comprising
CHA.9.547.18 HC
(EVQLVESGGGLVQPGGSLRLSCAASGFTFSSYIMSWVRQAPGK
GLEWVATISGGSKGQYYADSVKGRFTISRDNSKNTLYLQMNSL
RAEDTAVYYCAKWLLSYYAMDYWGQGTLVTVSS (SEQ ID
NO: 1664);
and
ii. a first light chain variable region comprising
CHA.9.547.18 VL
(DIQMTQSPSSLSASVGDRITITCRASQSMAIWLSWYQQKPGKA
PKLLIYKASKSHTGVPSRFSGSGSGTDFTLTISSLQPEDFATYYC
QQGQSYPYTFGQGTKLEIK; SEQ ID NO: 1668).

In some embodiments, the anti-PVRIG/anti-TIGIT bispecific antibody is a humanized antibody.

In some embodiments, the present invention provides a composition comprising an anti-PVRIG/anti-TIGIT bispecific antibody as described herein.

A nucleic acid composition comprising:
a) a first nucleic acid encoding a first heavy chain or heavy chain variable domain as described herein;
b) a second nucleic acid encoding a first light chain or light chain variable domain as described herein;
c) a third nucleic acid encoding a second heavy chain or heavy chain variable domain as described herein; and
d) a fourth nucleic acid encoding a second light chain or light chain variable domain as described herein.

In some embodiments, the present invention provides an expression vector composition comprising:
a) a first expression vector comprising the first nucleic acid as described herein;
b) a second expression vector comprising the second nucleic acid as described herein;
c) a third expression vector comprising the third nucleic acid as described herein; and
d) a fourth expression vector comprising the fourth nucleic acid as described herein.

In some embodiments, the present invention provides an expression vector composition comprising:
a) a first expression vector comprising the first and second nucleic acids as described herein; and
b) a second expression vector comprising the third and fourth nucleic acids as described herein.

In some embodiments, the present invention provides a host cell comprising the expression vector composition as described herein.

In some embodiments, the present invention provides a method of making an anti-PVRIG/anti-TIGIT bispecific antibody comprising:
a) culturing the host cell as described herein under conditions wherein the antibody is expressed; and
b) recovering the antibody.

In some embodiments, the present invention provides a method of activating T cells of a patient comprising administering the anti-PVRIG/anti-TIGIT bispecific antibody as described herein to the patient, wherein a subset of the T cells of the patient are activated.

In some embodiments, the present invention provides a method of activating cytotoxic T cells (CTLs) of a patient comprising administering the anti-PVRIG/anti-TIGIT bispecific antibody as described herein to the patient, wherein a subset of the CTLs of the patient are activated.

In some embodiments, the present invention provides a method of activating NK cells of a patient comprising administering the anti-PVRIG/anti-TIGIT bispecific antibody as described herein to the patient, wherein a subset of the NK cells of the patient are activated.

In some embodiments, the present invention provides a method of activating γδ T cells of a patient comprising administering the anti-PVRIG/anti-TIGIT bispecific antibody as described herein to the patient, wherein a subset of the γδ T cells of the patient are activated.

In some embodiments, the present invention provides a method of activating Th1 cells of a patient comprising administering the anti-PVRIG/anti-TIGIT bispecific antibody as described herein to the patient, wherein a subset of the Th1 cells of the patient are activated.

In some embodiments, the present invention provides a method of decreasing or eliminating cell number and/or activity of at least one of regulatory T cells (Tregs) in a patient comprising administering the anti-PVRIG/anti-TIGIT bispecific antibody as described herein to the patient.

In some embodiments, the present invention provides a method of increasing interferon-γ production and/or pro-inflammatory cytokine secretion in a patient comprising administering the anti-PVRIG/anti-TIGIT bispecific antibody as described herein to the patient.

In some embodiments, the present invention provides a method of treating cancer in a patient comprising administering the anti-PVRIG/anti-TIGIT antibody as described herein to the patient.

In some embodiments, the cancer is selected from the group consisting of prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung, non-small cell lung), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, esophageal cancer, Merkel Cells cancer, MSI-high cancer, KRAS mutant tumors, adult T-cell leukemia/lymphoma, myeloma, and Myelodysplastic syndromes (MDS).

In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma, and Myelodysplastic syndromes (MDS).

In some embodiments, the present invention provides an anti-PVRIG/anti-TIGIT bispecific antibody comprising:
a) a first antigen binding portion comprising an anti-PVRIG antigen binding portion comprising:
   i. a first heavy chain variable domain comprising a vhCDR1, vhCDR2, and vhCDR3 from an anti-PVRIG antibody; and
   ii. a first light chain variable domain comprising a vlCDR1, vlCDR2, and vlCDR3 from an anti-PVRIG antibody;
      wherein the anti-PVRIG antigen binding portion is selected from the group consisting of CPA.7.021, CPA.7.001, CPA.7.003, CPA.7.004, CPA.7.006, CPA.7.008, CPA.7.009, CPA.7.010, CPA.7.011, CPA.7.012, CPA.7.013, CPA.7.014, CPA.7.015, CPA.7.017, CPA.7.018, CPA.7.019, CPA.7.022, CPA.7.023, CPA.7.024, CPA.7.033, CPA.7.034, CPA.7.036, CPA.7.040, CPA.7.046, CPA.7.047, CPA.7.049, CPA.7.050, CHA.7.502, CHA.7.503, CHA.7.506, CHA.7.508, CHA.7.510, CHA.7.512, CHA.7.514, CHA.7.516, CHA.7.518, CHA.7.520.1, CHA.7.520.2, CHA.7.522, CHA.7.524, CHA.7.526, CHA.7.527, CHA.7.528, CHA.7.530, CHA.7.534, CHA.7.535, CHA.7.537, CHA.7.538.1, CHA.7.538.2, CHA.7.543, CHA.7.544, CHA.7.545, CHA.7.546, CHA.7.547, CHA.7.548, CHA.7.549, CHA.7.550, CHA.7.518.1; CHA7.538.1.2 and CHA.7.518.4; and
b) a second antigen binding portion comprising an anti-TIGIT antigen binding domain, wherein the anti-TIGIT antigen binding domain is from an antibody as provided in FIGS. 24A-24EE and 41A-41T, and in particular FIG. 24A-24EE.

In some embodiments, the present invention provides an anti-PVRIG/anti-TIGIT bispecific antibody comprising:
a) a first antigen binding portion comprising an anti-PVRIG antigen binding domain wherein the anti-PVRIG antigen binding domain is from an antibody as provided in FIG. 35A-35D; and
b) a second antigen binding portion comprising an anti-TIGIT antigen binding domain comprising:
   i. a second heavy chain variable domain comprising a vhCDR1, vhCDR2, and vhCDR3 from an anti-TIGIT antibody; and
   ii. a second light chain variable domain comprising a vlCDR1, vlCDR2, and vlCDR3 from an anti-TIGIT antibody;
      wherein the anti-TIGIT antigen binding domain is selected from the group consisting of CPA.9.018, CPA.9.027, CPA.9.049, CPA.9.057, CPA.9.059, CPA.9.083, CPA.9.086, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, CHA.9.541.8, and CHA.9.547.18.

In some embodiments, the present invention provides an anti-PVRIG antibody comprising:

i) a heavy chain or heavy chain variable domain
comprising the vhCDR1, vhCDR2, and vhCDR3
from the following sequence:

(SEQ ID NO: 3179)

QVQLVQSGAEVKKPGASVKVSCKASGYTFTDYNINWVRQAPGQGLEWMG
YIYPYIGGSGYAQKFQGRVTMTRDTSTSTVYMELSSLRSEDTAVYYCAR
EDKTARNAMDYWGQGTLVTVSS,
and
ii) a light chain or light chain variable domain
comprisingthe vlCDR1, vlCDR2, vlCDR3 from
the following sequence:

(SEQ ID NO: 3180)

DIQMTQSPSSLSASVGDRVTITCRVSENIYDVLAWYQQKPGKAPKLLIY
EATNLAEGVPSRFSGSGSGTDFTLTISSLQPEDFATYYCQHFWGTPYT
FGQGTKLEIK.

In some embodiments, the present invention provides an anti-TIGIT antibody comprising:

i) a heavy chain or heavy chain variable domain
comprising the vhCDR1, vhCDR2, and vhCDR3
from the following sequence:

(SEQ ID NO: 1664)

EVQLVESGGGLVQPGGSLRLSCAASGFTFSSYIMSWVRQAPGKGLEW
VATISGGSKGQYYADSVKGRFTISRDNSKNTLYLQMNSLRAEDTAVYYC
AKWLLSYYAMDYWGQGTLVTVSS,
and
ii) a light chain or light chain variable domain
comprising the vlCDR1, vlCDR2, and vlCDR3
from the following sequence:

(SEQ ID NO: 1668)

DIQMTQSPSSLSASVGDRITITCRASQSMAIWLSWYQQKPGKAPKLLIY
KASKSHTGVPSRFSGSGSGTDFTLTISSLQPEDFATYYCQQGQSYPYTF
GQGTKLEIK.

In some embodiments, the anti-PVRIG antibody comprises:

a) a heavy chain comprising CHA.7.518.4 VH
(QVQLVQSGAEVKKPGASVKVSCKASGYTFTDYNINWVRQAPGQGLE
WMGYIYPYIGGSGYAQKFQGRVTMTRDTSTSTVYMELSSLRSEDTAV
YYCAREDKTARNAMDYWGQGTLVTVSSASTKGPSVFPLAPCSRSTSE
STAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVEV
HNAKTKPREEQFNSTYRVVSVLTVLHQDWLNGKEYKCKVSNKGLPSS
IEKTISKAKGQPREPQVYTLPPCQDELTKNQVSLWCLVKGFYPSDIAV
EWESNGQPENNYKTTPPVLDSDGSFFLYSRLTVDKSRWQEGNVFSCS
VMHEALHNHYTQKSLSLSPGK; SEQ ID NO: 3175);
and
b) a light chain comprising CHA.7.518.4 VL
(DIQMTQSPSSLSASVGDRVTITCRVSENIYDVLAWYQQKPGKAPKLLI
YEATNLAEGVPSRFSGSGSGTDFTLTISSLQPEDFATYYCQHFWGTPYT
FGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYA
CEVTHQGLSSPVTKSFNRGEC; SEQ ID NO: 3362).

In some embodiments, the present invention provides a composition comprising an anti-PVRIG antibody according as described herein.

In some embodiments, the present invention provides a nucleic acid composition comprising:
i) a first nucleic acid encoding a heavy chain or heavy chain variable domain as described herein; and
ii) a second nucleic acid encoding a light chain or light chain variable domain as described herein.

In some embodiments, the present invention provides a composition comprising an anti-TIGIT antibody as described herein.

In some embodiments, the present invention provides a nucleic acid composition comprising:
i) a first nucleic acid encoding a heavy chain or heavy chain variable domain as described herein; and
ii) a second nucleic acid encoding a light chain or light chain variable domain as described herein.

In some embodiments, the present invention provides an expression vector composition comprising:
i) a first expression vector comprising the first nucleic acid as described herein; and
ii) a second expression vector comprising the second nucleic acid as described herein.

An expression vector comprising:
i) the first nucleic acid as described herein; and
ii) the second nucleic acid as described herein.

In some embodiments, the present invention provides an expression vector composition comprising:
i) a first expression vector comprising the first nucleic acid as described herein; and
ii) a second expression vector comprising the second nucleic acid as described herein.

In some embodiments, the present invention provides an expression vector comprising:
i) the first nucleic acid as described herein; and
ii) the second nucleic acid as described herein.

In some embodiments, the present invention provides a host cell comprising the expression vector or vector composition as described herein.

In some embodiments, the present invention provides a method of making an anti-PVRIG or anti-TIGIT antibody comprising:
i) culturing the host cell as described herein under conditions wherein the antibody is expressed; and
ii) recovering the antibody.

In some embodiments, the present invention provides a method of activating T cells of a patient comprising administering the anti-PVRIG or anti-TIGIT antibody as described herein to the patient, wherein a subset of the T cells of the patient are activated.

In some embodiments, the present invention provides a method of activating cytotoxic T cells (CTLs) of a patient comprising administering the anti-PVRIG or anti-TIGIT antibody as described herein to the patient, wherein a subset of the CTLs of the patient are activated.

In some embodiments, the present invention provides a method of activating NK cells of a patient comprising administering the anti-PVRIG or anti-TIGIT antibody as described herein to the patient, wherein a subset of the NK cells of the patient are activated.

In some embodiments, the present invention provides a method of activating γδ T cells of a patient comprising administering the anti-PVRIG or anti-TIGIT antibody as described herein to the patient, wherein a subset of the γδ T cells of the patient are activated.

In some embodiments, the present invention provides a method of activating Th1 cells of a patient comprising administering the anti-PVRIG or anti-TIGIT antibody as described herein to the patient, wherein a subset of the Th1 cells of the patient are activated.

In some embodiments, the present invention provides a method of decreasing or eliminating cell number and/or activity of at least one of regulatory T cells (Tregs) in a patient comprising administering the anti-PVRIG or anti-TIGIT antibody as described herein to the patient.

In some embodiments, the present invention provides a method of increasing interferon-γ production and/or pro-inflammatory cytokine secretion in a patient comprising administering the anti-PVRIG or anti-TIGIT antibody as described herein to the patient.

In some embodiments, the present invention provides a method of treating cancer in a patient comprising administering the anti-PVRIG or anti-TIGIT antibody as described herein to the patient.

In some embodiments, the cancer is selected from the group consisting of prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung, non-small cell lung), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, esophageal cancer, Merkel Cells cancer, MSI-high cancer, KRAS mutant tumors, adult T-cell leukemia/lymphoma, myeloma, and Myelodysplastic syndromes (MDS).

In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma, and Myelodysplastic syndromes (MDS).

In some embodiments, the present invention provides a method of treating cancer in a patient comprising administering a combination therapy comprising an anti-PVRIG/anti-TIGIT bispecific antibody according to as described herein.

In some embodiments, the present invention provides a method of treating cancer in a patient comprising administering a combination therapy comprising an anti-PVRIG antibody as described herein and an anti-PD-1 antibody.

In some embodiments, the anti-PD-1 antibody is an antibody selected from the group consisting of pembrolizumab and nivolumab.

In some embodiments, the present invention provides a method of treating cancer in a patient comprising administering a combination therapy comprising an anti-TIGIT antibody as described herein and an anti-PD-1 antibody.

In some embodiments, the anti-PD-1 antibody is an antibody selected from the group consisting of pembrolizumab and nivolumab.

In some embodiments, the present invention provides a method of treating cancer in a patient comprising administering a combination therapy comprising an anti-TIGIT antibody as described herein and an anti-PVRIG antibody as described herein.

In some embodiments, the present invention provides a method of treating cancer in a patient comprising administering a triple combination therapy comprising an anti-TIGIT antibody as described herein, an anti-PVRIG antibody, and an anti-PD-1 antibody.

In some embodiments, the present invention provides a method of treating cancer in a patient comprising administering a triple combination therapy comprising an anti-TIGIT, an anti-PVRIG antibody as described herein, and an anti-PD-1 antibody.

In some embodiments, the present invention provides a method of treating cancer in a patient comprising administering a triple combination therapy comprising an anti-TIGIT as described herein, an anti-PVRIG antibody as described herein, and an anti-PD-1 antibody.

In some embodiments, the anti-PD-1 antibody is an antibody selected from the group consisting of pembrolizumab and nivolumab.

In some embodiments, the present invention provides a method of activating T cells of a patient comprising administering the anti-PVRIG or anti-TIGIT antibody as described herein to the patient, wherein a subset of the T cells of the patient are activated.

In some embodiments, the present invention provides a method of activating cytotoxic T cells (CTLs) of a patient comprising administering the anti-PVRIG or anti-TIGIT antibody as described herein to the patient, wherein a subset of the CTLs of the patient are activated.

In some embodiments, the present invention provides a method of activating γδ T cells of a patient comprising administering the anti-PVRIG or anti-TIGIT antibody as described herein to the patient, wherein a subset of the γδ T cells of the patient are activated.

In some embodiments, the present invention provides a method of activating Th1 cells of a patient comprising administering the anti-PVRIG or anti-TIGIT antibody as described herein to the patient, wherein a subset of the Th1 cells of the patient are activated.

In some embodiments, the present invention provides a method of decreasing or eliminating cell number and/or activity of at least one of regulatory T cells (Tregs) in a patient comprising administering the anti-PVRIG or anti-TIGIT antibody as described herein to the patient.

In some embodiments, the present invention provides a method of increasing interferon-γ production and/or pro-inflammatory cytokine secretion in a patient comprising administering the anti-PVRIG or anti-TIGIT as described herein to the patient.

In some embodiments, the present invention provides a method of treating cancer in a patient comprising administering the anti-PVRIG or anti-TIGIT antibody as described herein to the patient.

In some embodiments, the cancer is selected from the group consisting of prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung, non-small cell lung), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, esophageal cancer, Merkel Cells cancer, MSI-high cancer, KRAS mutant tumors, adult T-cell leukemia/lymphoma, myeloma, and Myelodysplastic syndromes (MDS).

In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma, and Myelodysplastic syndromes (MDS).

### III. BRIEF DESCRIPTION OF THE DRAWINGS

FIG. 1 depicts the sequences of human PVRIG (showing two different methionine starting points as well as the full length sequence). The signal peptide is underlined, the ECD is double underlined. PVRIG, also called Poliovirus Receptor Related Immunoglobulin Domain Containing Protein, Q6DKI7 or C7orf15, relates to amino acid and nucleic acid sequences shown in RefSeq accession identifier NP_076975, shown in FIG. 1.

FIG. 2 depicts the sequence of the human Poliovirus receptor-related 2 protein (PVLR2, also known as nectin-2, CD112 or herpesvirus entry mediator B, (HVEB)), the binding partner of PVRIG. PVLR2 is a human plasma membrane glycoprotein.

FIG. 3A-3C shows the CDR sequences for Fabs that were determined to successfully block interaction of the PVRIG with its counterpart PVRL2.

FIGS. 4A-4AA shows the amino acid sequences of the variable heavy and light domains, the full length heavy and light chains, and the variable heavy and variable light CDRs for the enumerated human CPA anti-PVRIG sequences of the invention that both bind PVRIG and block binding of PVRIG and PVLR2.

FIGS. 5A-5H depicts the amino acid sequences of the variable heavy and light domains, the full length heavy and light chains, and the variable heavy and variable light CDRs for eight human CPA anti-PVRIG sequences of the invention that bind PVRIG and but do not block binding of PVRIG and PVLR2.

FIGS. 6A-6G depicts the CDRs for all CPA anti-PVRIG antibody sequences that were generated that bind PVRIG, including those that do not block binding of PVRIG and PVLR2.

FIGS. 7A-7AE depicts the variable heavy and light chains as well as the vhCDR1, vhCDR2, vhCDR3, vlCDR1, vlCDR2 and vlCDR3 sequences of each of the enumerated CHA antibodies of the invention, CHA.7.502, CHA.7.503, CHA.7.506, CHA.7.508, CHA.7.510, CHA.7.512, CHA.7.514, CHA.7.516, CHA.7.518, CHA.7.520.1, CHA.7.520.2, CHA.7.522, CHA.7.524, CHA.7.526, CHA.7.527, CHA.7.528, CHA.7.530, CHA.7.534, CHA.7.535, CHA.7.537, CHA.7.538.1, CHA.7.538.2, CHA.7.543, CHA.7.544, CHA.7.545, CHA.7.546, CHA.7.547, CHA.7.548, CHA.7.549, CHA.7.550, and CHA.7.518.4 (these include the variable heavy and light sequences from mouse sequences (from Hybridomas).

FIGS. 8A-8D depicts the vhCDR1, vhCDR2, vhCDR3, vlCDR1, vlCDR2 and vlCDR3 sequences of each of the enumerated CPA antibodies of the invention, CPA.7.001 to CPA.7.050 are human sequences (from Phage display).

FIGS. 9A-9C depicts the sequences of human IgG1, IgG2, IgG3 and IgG4.

FIG. 10 depicts a number of human PVRIG ECD fragments.

FIGS. 11A-11I depicts a collation of the humanized sequences of five CHA antibodies.

FIGS. 12A-12E depicts a collation of the humanized sequences of five CHA antibodies.

FIG. 13 depicts schemes for combining the humanized VH and VL CHA antibodies of FIGS. 11A-11I and FIGS. 12A-12E. The “chimVH” and “chimVL” are the mouse variable heavy and light sequences attached to a human IgG constant domain.

FIGS. 14A-14BX show a number of PVRIG sequences and other sequences that find use in the invention.

FIGS. 15A and 15B depict the variable heavy and light chains as well as the vhCDR1, vhCDR2, vhCDR3, vlCDR1, vlCDR2 and vlCDR3 sequences of each of the enumerated CHA antibodies of the invention, CHA.7.518.1.H4(S241P), and CHA.7.538.1.2.H4(S241P).

FIG. 16A-16E depict four humanized sequences for each of CHA.7.518, CHA.7.524, CHA.7.530, CHA.7.538_1 and CHA.7.538_2. All humanized antibodies comprise the H4(S241P) substitution. Note that the light chain for CHA.7.538_2 is the same as for CHA.7.538_1. The “H1” of each is a “CDR swap” with no changes to the human framework. Subsequent sequences alter framework changes shown in larger bold font. CDR sequences are noted in bold. CDR definitions are AbM from website www.bioinf.org.uk/abs/. Human germline and joining sequences from IMGT® the international ImMunoGeneTics® information system www.imgt.org (founder and director: Marie-Paule Lefranc, Montpellier, France). Residue numbering shown as sequential (seq) or according to Chothia from website www.bioinf.org.uk/abs/(AbM). “b” notes buried sidechain; “p” notes partially buried; “i” notes sidechain at interface between VH and VL domains. Sequence differences between human and murine germlines noted by asterisk (*). Potential additional mutations in frameworks are noted below sequence. Potential changes in CDR sequences noted below each CDR sequence as noted on the figure (# deamidation substitutions: Q/S/A; these may prevent asparagine (N) deamidation. @ tryptophan oxidation substitutions: Y/F/H; these may prevent tryptophan oxidation; @ methionine oxidation substitutions: L/F/A).

FIG. 17A to 17C depicts a collation of the humanized sequences of three CHA antibodies: CHA.7.518, CHA.7.538.1, and CHA.7.538.2.

FIG. 18 depicts schemes for combining the humanized VH and VL CHA antibodies. The “chimVH” and “chimVL” are the mouse variable heavy and light sequences attached to a human IgG constant domain.

FIG. 19. Sequence alignment of PVRIG orthologs. Aligned sequences of the human, cynomolgus, marmoset, and rhesus PVRIG extra-cellular domain. The differences between human and cynomolgus are highlighted in yellow.

FIG. 20A-20D depict the amino acid sequences and the nucleic acid sequence for the variable heavy chain (A and B, respectfully) and the amino acid sequences and the nucleic acid sequence for the variable light chain (C and D, respectfully) for AB-407 (BOJ-5G4-F4).

FIGS. 21A and 21B depicts the amino acid sequences of the constant domains of human IgG1 (with some useful amino acid substitutions), IgG2, IgG3, IgG4, IgG4 with a hinge variant that finds particular use in the present invention, and the constant domains of the kappa and lambda light chains.

FIG. 22 depicts the sequences of human and cynomolgus macaque (referred to as cyno) TIGIT ECD and of the human PVR ECD proteins.

FIG. 23A-23D depict the sequences of anti-TIGIT antibodies. Unless otherwise noted, the CDRs utilize the IMGT numbering (including the antibodies of the sequence listing).

FIG. 24A-24EE depict the sequences of numerous anti-TIGIT antibodies for use in the bispecifc antibodies of the present invention.

FIG. 25 depicts a schematic of an exemplary bispecific antibody embodiment using the CrossMab Technology (MAbs. 2016 August-September; 8(6): 1010-1020) and knobs in hole technology (Nat Biotechnol. 1998 July; 16(7):677-81).

FIG. 26A-26B provides the sequences of the heavy and light chains for each arm of the CHA.7.518.1.H4(S241P)/CPA.9.086 bispecific antibody. The variable domains are shown in italics. The CDRs are colored in red. The mutations in the Fc domains are shown in bold and underlined.

FIG. 27 provides the SPR sensorgrams (black lines) of (A) human TIGIT binding to captured TIGIT-PVRIG Bispecific antibody over two independent surfaces and of (B) human PVRIG binding to captured TIGIT-PVRIG Bispecific antibody over two independent surfaces. The red lines in both (A) and (B) are the global fit of the sensorgrams to a simple 1:1 kinetic binding model. The concentration range of TIGIT was 362 pM-88 nM and the concentration range of PVRIG was 460 pM-112 nM. Because there wasn't sufficient dissociation signal decay data after 15 minutes with the TIGIT antigen, the k_{d} was arbitrarily held constant at 1e⁻⁵ sec⁻¹ for the global fit. When holding kd constant for the TIGIT-bispecific interaction, the ka was estimated to be 3.9e⁶ M⁻¹ sec⁻¹ with the k_{d}/kₐ ratio calculating K_{D}=2.6 pM. The binding constants for the PVRIG-Bispecific antibody interaction were kₐ=1.3e⁶ M⁻¹ sec⁻¹, k_{d}=2.4e⁻⁴ sec⁻¹, and K_{D}=187 pM.

FIG. 28 depicts SPR “sandwich” assay demonstrating simultaneous binding of human TIGIT and human PVRIG to the anti-TIGIT-PVRIG bispecific antibody. The bispecific antibody is first injected (A) over human TIGIT covalently immobilized to the biosensor chip. Human PVRIG is then injected (B) over the TIGIT-bound bispecific antibody.

FIGS. 29A-29B depicts characterization of pp65 specific CD8⁺ T cells. A) PBMCs were activated with pp65₍₄₉₅₋₅₀₃₎ peptide, IL-2 and IL-7 for 11 days. Flow cytometry was performed to assess the percentage of pp65 reactive T cells and the expression of PVRIG, TIGIT, and PD-1. Representative gating hierarchy from 1 donor and representative tetramer staining from two donors are shown. From left to right, lymphocytes were gated by forward scatter (FSC)/side scatter (SSC) (upper left), and live CD3⁺CD8⁺ positive cells. Within the CD3⁺CD8⁺ positive population, the percentage of cells that bind HLA-A201 pp65 tetramer is determined and data from 2 donors are shown. B. The expression of PVRIG, TIGIT and PD-1 on the pp65 reactive T cells was evaluated by flow cytometry. The number in the upper right-hand corner denotes the MFIr of the target of interest relative to isotype control.

FIG. 30 depicts the effect of inhibitory receptor blockade on CMV pp65 reactive CD8 T cells in co-culture with Mel-624 pp65 cancer cell line. CMV pp65 reactive T cells for 2 donors, Donor 4 (A) and Donor 72 (B) were co-cultured with a modified Mel-624 tumor cell line ectopically expressing pp65 in the presence of CHA.7.518.1.H4(S241P) and CPA.9.086 either alone or in combination, CHA.7.518.1.H4(S241P)/CPA.9.086 BsAb or a human IgG isotype control for 18 hrs. Conditioned media were assayed for cytokine secretion. The numbers above the bars indicate the % change relative to isotype control. The bar graphs show the average±standard deviation for IFN-γ (N=2).

FIG. 31A-31B provides dose-dependent titration of inhibitory receptor blockade on CMV pp65 reactive CD8 T cells in co-culture with Mel-624 pp65 cancer cell line. CMV pp65 reactive T cells for 2 donors, Donor 4 (A) and Donor 72 (B) were co-cultured with a modified Mel-624 tumor cell line ectopically expressing pp65 in the presence of CHA.7.518.1.H4(S241P) and CPA.9.086 either alone or in combination, CHA.7.518.1.H4(S241P)/CPA.9.086 BsAb or a human IgG isotype control for 18 hrs. A 10 point, 4-fold dilution series starting at 20 μg/ml was used for each antibody. Conditioned media were assayed for cytokine secretion. EC₅₀ (nM) values for combined blockade using CHA.7.518.1.H4(S241P) and CPA.9.086 and for the bispecific antibody treatments are reported. Representative data (N=2).

FIG. 32 provides an overview of Fc heterodimerzation strategies for use in bispecific antibody generation applicable to the bispecific antibodies of the invention. (See, Godar, et. al., Expert Opinion on Therapeutic Patents, 28(3):251-276 (2018).)

FIG. 33 provides an overview of asymmetric bispecific antibody formats applicable to the bispecific antibodies of the invention. (See, Brinkmann and Kontermann, The making of bispecific antibodies, MAbs, 9(2): 182-212 (2017).)

FIG. 34 provides an overview of symmetric bispecific antibody formats applicable to the bispecific antibodies of the invention. (See, Brinkmann and Kontermann, The making of bispecific antibodies, MAbs, 9(2): 182-212 (2017).)

FIG. 35A-35D provides additional anti-PVRIG antibodies for use in the bispecifc antibodies of the present invention.

FIG. 36 provides the SPR sensorgrams (black lines) of human PVRIg binding to captured PVRIg bispecific and monospecific antibodies. The red lines in both are the global fit of the sensorgrams to a simple 1:1 kinetic binding model. The concentration range of PVRIG was 460 pM-112 nM. (A) CHA.7.518.4-H4 hole+CHA.9.547.18-H4 CrossMab knob, (B) CHA.7.518.4-H4 hole+CPA.9.086-CrossMab H4 knob, (C) CHA.7.518.4-H4 hole+CPA.9.086 scFv (VL-VH)-H4 knob, (D) CHA.7.518.4-H4 hole+CPA.9.086 scFv (VH-VL)-H4 knob, (E) CPA.9.086 scFv (VH-VL) H4 var1+CHA.7.518.1 Fab H4 var2, (F) CHA.7.518.1 scFv (VL-VH) H4 var1+CPA.9.086 Fab H4 var2, (G) CHA.9.547.18 scFv (VH-VL) H4 var1+CHA.7.518.4 Fab H4 var2, (H) CHA.7.518.4 scFv (VL-VH) H4 var1+CHA.9.547.18 Fab H4 var2, (I) CHA.7.518.1 H4, (I) (J) Synagis H4. The binding affinities were estimated from the k_{d}/kₐ ratio (K_{D}) calculation for each antibody and are presented in FIG. 38.

FIG. 37 provides the SPR sensorgrams (black lines) of human TIGIT binding to captured TIGIT bispecific and monospecific antibodies. The red lines are the global fit of the sensorgrams to a simple 1:1 kinetic binding model. The concentration range of TIGIT was 362 pM-88 nM. (A) CHA.7.518.4-H4 hole+CHA.9.547.18-H4 CrossMab knob, (B) CHA.7.518.4-H4 hole+CPA.9.086-CrossMab H4 knob, (C) CHA.7.518.4-H4 hole+CPA.9.086 scFv (VL-VH)-H4 knob, (D) CHA.7.518.4-H4 hole+CPA.9.086 scFv (VH-VL)-H4 knob, (E) CPA.9.086 scFv (VH-VL) H4 var1+CHA.7.518.1 Fab H4 var2, (F) CHA.7.518.1 scFv (VL-VH) H4 var1+CPA.9.086 Fab H4 var2, (G) CHA.9.547.18 scFv (VH-VL) H4 var1+CHA.7.518.4 Fab H4 var2, (H) CHA.7.518.4 scFv (VL-VH) H4 var1+CHA.9.547.18 Fab H4 var2, (I) CPA.9.086 H4, (J) Synagis H4. The binding affinities were estimated from the k_{d}/kₐ ratio (K_{D}) calculation for each antibody and are presented in FIG. 39.

FIG. 38 provides the PVRIG binding affinities of bispecific and monospecific antibodies determined by SPR.

FIG. 39 provides the TIGIT binding affinities of bispecific and monospecific antibodies determined by SPR.

FIG. 40A-40T provides additional anti-PVRIG antibodies for use in the bispecifc antibodies of the present invention. Red font text indicates amino acid substitutions and ( ) indicates a deletion relative to the reference human IgG4 amino acid sequence. Underlined text indicates CDRs. Grey highlighted text indicates the Fc domains.

FIG. 41A-41T provides additional anti-TIGIT antibodies for use in the bispecifc antibodies of the present invention. Red font text indicates amino acid substitutions and ( ) indicates a deletion relative to the reference human IgG4 amino acid sequence. Underlined text indicates CDRs. Grey highlighted text indicates the Fc domains.

FIG. 42 provides a diagram of an exemplary CrossMab bispecific antibody format that has one traditional antibody arm and one in which the heavy chain and light chain constant domains are swapped. Amino acid substitutions for the “knob into hole” format are indicated according to human IgG1 Eu numbering.

FIG. 43 provides a diagram of an exemplary “bottle opener” bispecific antibody format that has one traditional antibody arm and one scFv-Fc fusion arm. Amino acid substitutions for the “knob into hole” format are indicated according to human IgG1 Eu numbering.

FIG. 44 provides a diagram of an exemplary “bottle opener” bispecific antibody format that has one traditional antibody arm and one scFv-Fc fusion arm. Amino acid substitutions for the “isovolumetric heterodimerization” format are indicated according to human IgG1 Eu numbering. Additional substitutions that contribute to reduced FcgR binding are indicated.

FIG. 45 provides data regarding the expression and purification of anti-PVRIG-TIGIT bispecific antibodies, monomer content and % correct assembled bispecific antibody as determined by LC-MS after MabSelect Sure affinity chromatography step and size exclusion chromatography (if % monomer was less than 95% from affinity chromatography).

FIG. 46A-46C provides data regarding CMV screening assays for anti-PVRIG-TIGIT bispecific antibody screening on three different T-cell donors and performed at 10 ug/ml total antibody concentration per treatment

FIG. 47 provides flow cytometry examples of PVRIG, TIGIT and PD-1 ligand (PVR, PVRL2 and PD-L1, respectively) expression on recombinant CHO cell lines used in the cell assays.

FIG. 48 provides flow cytometry examples of PVRIG, TIGIT and PD-1 expression levels in T-cells from three different donors.

FIG. 49 provides ELISA binding data demonstrating simultaneous binding of both PVRIG and TIGIT for various anti-PVRIG-TIGIT antibodies using a PVRIG coated plate and detecting bound antibodies using biotinylated TIGIT-His and Steptavidin-HRP.

FIG. 50 provides ELISA binding data demonstrating simultaneous binding of both PVRIG and TIGIT for various anti-PVRIG-TIGIT antibodies using a TIGIT coated plate and detecting bound antibodies using biotinylated PVRIG-His and Steptavidin-HRP.

FIG. 51 provides EC-50 data for the ELISA assay data provided in FIGS. 49 and 50.

FIG. 52 provides example stability data of bispecific antibodies in multiple formats as assessed by Differential Scanning Fluorimetry (DSF) and aggregate formation after a low pH hold and 3 cycles of freezing and thaw.

FIG. 53A-53D provides stability assessment of the different bispecific formats. Tests were performed to simulate viral inactivation by holding the antibodies at pH 3 for various times and to stress test by performing 3 serial freeze/thaw cycles. Changes in the relative amount of heavy chain (scFv or Fab-containing) (A), light chain (B) under reducing conditions or intact molecule (C) in non-reducing conditions (CE-SDS) and in low molecular weight, high molecular weigh, and monomer species content (SEC-UPLC) (D). Data are reported as a change from T=0.

FIG. 54A-54B FIG. 54A provides an in vitro co-culture assay with human CMV-specific CD8+ T cells from three donors (Donor 4, Donor 210, and Donor 234) were utilized to assess the effect of anti-TIGIT antibodies (CPA.9.086 and CHA.9.547.18) and anti-PVRIG antibodies (CHA.7.518.1, CHA.7.518.4) on antigen-specific cytokine secretion either alone, in combination or as bispecific antibodies. The target cell line used in the assay was the HLA-A2+PVRL2+PVR+expressing melanoma 624 (Mel-624) cell line which has been modified to ectopically express pp65. Cells were plated at 75,000 cells/well in 96-well round-bottom tissue culture treated plates. 15,000 human CD8+ T cells were added to each well. The indicated anti-human PVRIG, TIGIT or isotype control hIgG4 antibodies were added at a concentration of 1 μg/ml. Co-cultures were incubated at 37° C. with 5% CO2 for 24 hours. The amount of human IFNγ in the co-culture supernatant was measured by flow cytometry using a LEGENDplex IFN bead assay (BD Biosciences). The percent increase of IFNγ secretion for each antibody over the hIgG4 isotype is indicated (n=2 experiments, representative results from 1 experiment shown). (A) CHA.7.518.4-H4 hole+CPA.9.086-CrossMab H4 knob, (B) CHA.7.518.4-H4 hole+CPA.9.086 scFv (VH-VL)-H4 knob, (C) CPA.9.086 scFv (VH-VL) H4 var1+CHA.7.518.1 Fab H4 var2, (D) CHA.7.518.1 scFv (VL-VH) H4 var1+CPA.9.086 Fab H4 var2, (E) CHA.7.518.1 H4, (F) CPA.9.086 H4, (G) CHA.7.518.1 H4, (H) CPA.9.086 H4, (I) Synagis H4. FIG. 54B provides a summary of the two CMV assay replicates performed with different expansions of the donor cells.

### IV. DETAILED DESCRIPTION OF THE INVENTION

### A. Overview

The present invention provides a number of useful anti-PVRIG, anti-TIGIT, and/or anti-PVRIG/anti-TIGIT bispecific antibodies, for use in particular in the treatment of cancer. Cancer can be considered as an inability of the patient to recognize and eliminate cancerous cells. In many instances, these transformed (e.g. cancerous) cells counteract immunosurveillance. There are natural control mechanisms that limit T-cell activation in the body to prevent unrestrained T-cell activity, which can be exploited by cancerous cells to evade or suppress the immune response. Restoring the capacity of immune effector cells-especially T cells-to recognize and eliminate cancer is the goal of immunotherapy. The field of immuno-oncology, sometimes referred to as “immunotherapy” is rapidly evolving, with several recent approvals of T cell checkpoint inhibitory antibodies such as Yervoy, Keytruda and Opdivo. These antibodies are generally referred to as “checkpoint inhibitors” because they block normally negative regulators of T cell immunity. It is generally understood that a variety of immunomodulatory signals, both costimulatory and coinhibitory, can be used to orchestrate an optimal antigen-specific immune response. Generally, these antibodies bind to checkpoint inhibitor proteins such as CTLA-4 or PD-1, which under normal circumstances prevent or suppress activation of cytotoxic T cells (CTLs). By inhibiting the checkpoint protein, for example through the use of antibodies that bind these proteins, an increased T cell response against tumors can be achieved. That is, these cancer checkpoint proteins suppress the immune response; when the proteins are blocked, for example using antibodies to the checkpoint protein, the immune system is activated, leading to immune stimulation, resulting in treatment of conditions such as cancer and infectious disease.

The present invention is directed to the use of bispecific antibodies to additional checkpoint proteins, PVRIG and TIGIT. PVRIG is expressed on the cell surface of NK and T-cells and shares several similarities to other known immune checkpoints. The identification and methods used to show that PVRIG is a checkpoint receptor are discussed in WO2016/134333, expressly incorporated herein by reference. Anti-PVRIG, anti-TIGIT, and/or anti-PVRIG/anti-TIGIT bispecific antibodies to human PVRIG that block the interaction and/or binding of PVLR2 are provided herein. When PVRIG is bound by its ligand (PVRL2), an inhibitory signal is elicited which acts to attenuate the immune response of NK and T-cells against a target cell (i.e. analogous to PD-1/PDL1). Blocking the binding of PVRL2 to PVRIG shuts-off this inhibitory signal of PVRIG and as a result modulates the immune response of NK and T-cells. Utilizing an antibody against PVRIG that blocks binding to PVRL2 is a therapeutic approach that enhances the killing of cancer cells by NK and T-cells. Blocking antibodies have been generated which bind PVRIG and block the binding of its ligand, PVRL2.

Similarly, TIGIT has been shown to also have attributes of a checkpoint receptor, and the present invention provides anti-PVRIG, anti-TIGIT, and/or anti-PVRIG/anti-TIGIT bispecific antibodies that block the interaction and/or binding of TIGIT to PVR are provided. When TIGIT is bound by its ligand (PVR), an inhibitory signal is elicited which acts to attenuate the immune response of NK and T-cells against a target cell (i.e. analogous to PD-1/PDL1). Blocking the binding of PVR to TIGIT shuts-off this inhibitory signal of TIGIT and as a result modulates the immune response of NK and T-cells. Utilizing an antibody against TIGIT that blocks binding to PVR is a therapeutic approach that enhances the killing of cancer cells by NK and T-cells. Blocking antibodies have been generated which bind TIGIT and block the binding of its ligand, PVR.

Additionally, the invention provides anti-PVRIG, anti-TIGIT, and/or anti-PVRIG/anti-TIGIT bispecific antibodiesfor use in the treatment of cancer.

### B. Definitions

In order that the application may be more completely understood, several definitions are set forth below. Such definitions are meant to encompass grammatical equivalents.

IgG domain definitions used herein are in accordance with IMGT reference sequences (www.IMGT.org)

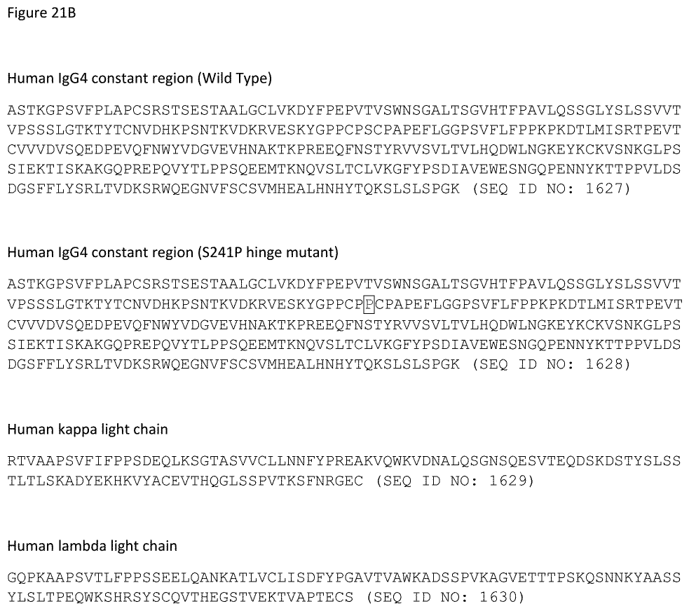

By “ablation” herein is meant a decrease or removal of activity. In some embodiments, it is useful to remove activity from the constant domains of the antibodies. Thus, for example, “ablating FcγR binding” means the Fc region amino acid variant has less than 50% starting binding as compared to an Fc region not containing the specific variant, with less than 70-80-90-95-98% loss of activity being preferred, and in general, with the activity being below the level of detectable binding in a Biacore assay. As shown in FIG. 21A-21B, one ablation variant in the IgG1 constant region is the N297A variant, which removes the native glycosylation site and significantly reduces the FcγRIIIa binding and thus reduces the antibody dependent cell-mediated cytotoxicity (ADCC).

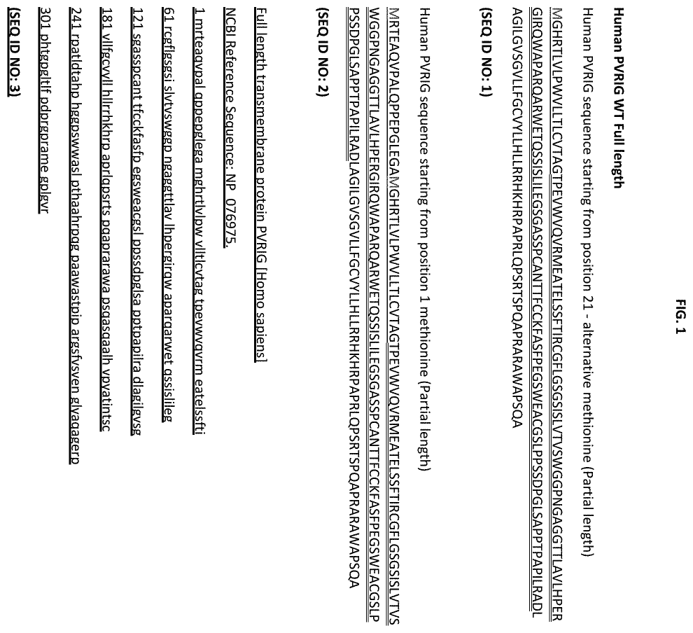

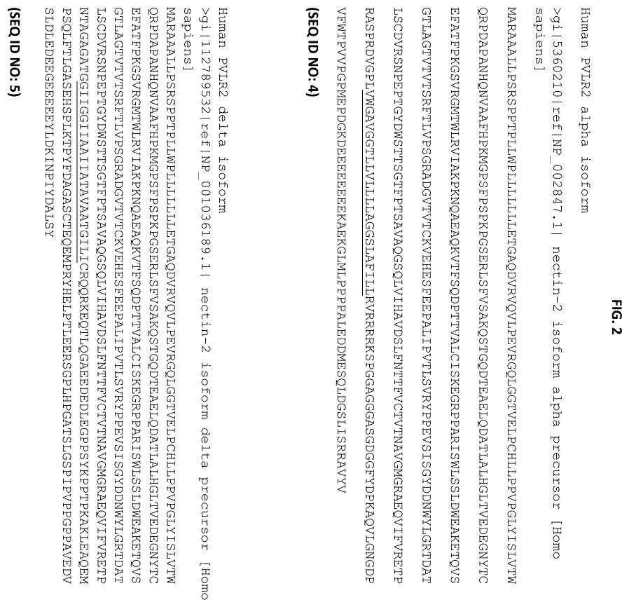

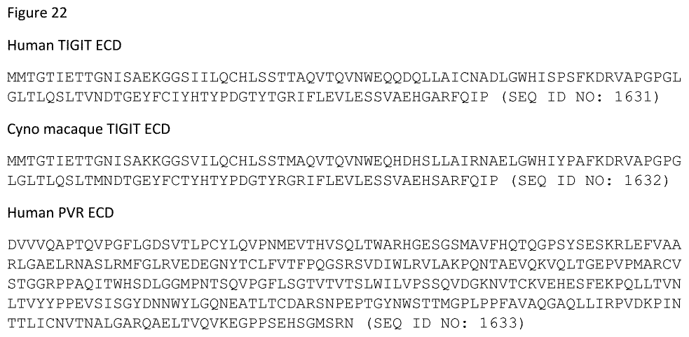

By “antigen binding domain” or “ABD” herein is meant a set of six Complementary Determining Regions (CDRs) that, when present as part of a polypeptide sequence, specifically binds a target antigen as discussed herein. Thus, a “TIGIT antigen binding domain” binds TIGIT antigen (the sequence of which is shown in FIG. 22) as outlined herein. Similarly, a “PVRIG antibody binding domain” binds PVRIG antigen (the sequence of which is shown in FIG. 1) as outlined herein. As is known in the art, these CDRs are generally present as a first set of variable heavy CDRs (vhCDRs or V_{H}CDRs) and a second set of variable light CDRs (vlCDRs or V_{L}CDRs), each comprising three CDRs: vhCDR1, vhCDR2, vhCDR3 for the heavy chain and vlCDR1, vlCDR2 and vlCDR3 for the light. The CDRs are present in the variable heavy and variable light domains, respectively, and together form an Fv region. Thus, in some cases, the six CDRs of the antigen binding domain are contributed by a variable heavy and variable light chain. In a “Fab” format, the set of 6 CDRs are contributed by two different polypeptide sequences, the variable heavy domain (vh or V_{H}; containing the vhCDR1, vhCDR2 and vhCDR3) and the variable light domain (vl or V_{L}; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). The phrase “antigen binding portion” can comprise an ABD or be synonymous with ABD.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g. the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution N297A refers to a variant polypeptide, in this case an Fc variant, in which the asparagine at position 297 is replaced with alanine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233− or E233#, E233( ) or E233del designates a deletion of glutamic acid at position 233. Additionally, EDA233− or EDA233# designates a deletion of the sequence GluAspAla that begins at position 233.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g. from about one to about seventy amino acid modifications, and preferably from about one to about five amino acid modifications compared to the parent. As described below, in some embodiments the parent polypeptide, for example an Fc parent polypeptide, is a human wild type sequence, such as the Fc region from IgG1, IgG2, IgG3 or IgG4, although human sequences with variants can also serve as “parent polypeptides”. The protein variant sequence herein will preferably possess at least about 80% identity with a parent protein sequence, and most preferably at least about 90% identity, more preferably at least about 95-98-99% identity. Variant protein can refer to the variant protein itself, compositions comprising the protein variant, or the DNA sequence that encodes it. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain. The Fc variants of the present invention are defined according to the amino acid modifications that compose them. Thus, for example, S241P or S228P is a hinge variant with the substitution proline at position 228 relative to the parent IgG4 hinge polypeptide, wherein the numbering S228P is according to the EU index and the S241P is the Kabat numbering. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. Substitutions can include naturally occurring amino acids and, in some cases, synthetic amino acids. Examples include U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all entirely incorporated by reference.

As used herein, “protein” herein is meant at least two covalently attached amino acids, which includes proteins, polypeptides, oligopeptides and peptides. The peptidyl group may comprise naturally occurring amino acids and peptide bonds, or synthetic peptidomimetic structures, e.g., “analogs”, such as peptoids (see Simon et al., PNAS USA 89(20):9367 (1992), entirely incorporated by reference). The amino acids may either be naturally occurring or synthetic (e.g. not an amino acid that is coded for by DNA); as will be appreciated by those in the art. For example, homo-phenylalanine, citrulline, ornithine and noreleucine are considered synthetic amino acids for the purposes of the invention, and both D- and L- (R or S) configured amino acids may be utilized. The variants of the present invention may comprise modifications that include the use of synthetic amino acids incorporated using, for example, the technologies developed by Schultz and colleagues, including but not limited to methods described by Cropp & Shultz, 2004, Trends Genet. 20(12):625-30, Anderson et al., 2004, Proc Natl Acad Sci USA 101 (2):7566-71, Zhang et al., 2003, 303(5656):371-3, and Chin et al., 2003, Science 301(5635):964-7, all entirely incorporated by reference. In addition, polypeptides may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “Fab” or “Fab region” as used herein is meant the polypeptide that comprises the VH, CH1, VL, and CL immunoglobulin domains. Fab may refer to this region in isolation, or this region in the context of a full length antibody or antibody fragment.

By “Fv” or “Fv fragment” or “Fv region” as used herein is meant a polypeptide that comprises the VL and VH domains of a single antibody. As will be appreciated by those in the art, these generally are made up of two chains.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In general, the linker is a scFv linker as is generally known in the art, with the linker peptide predominantly including the following amino acid residues: Gly, Ser, Ala, or Thr.

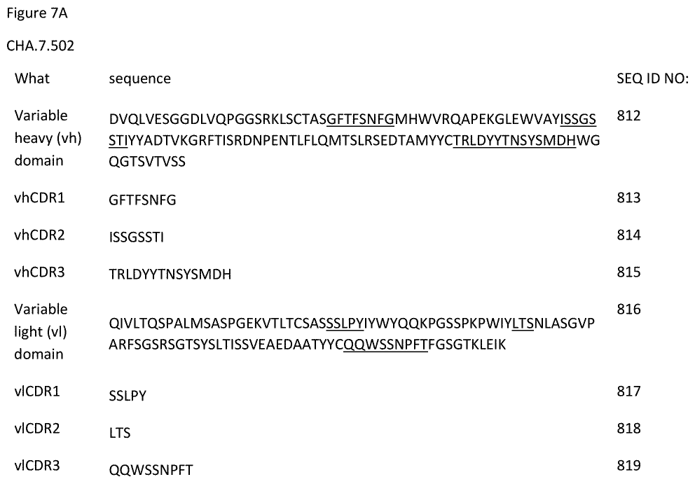

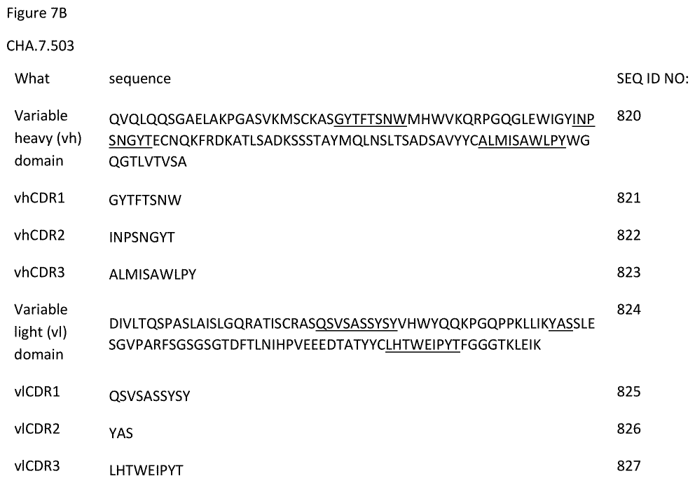

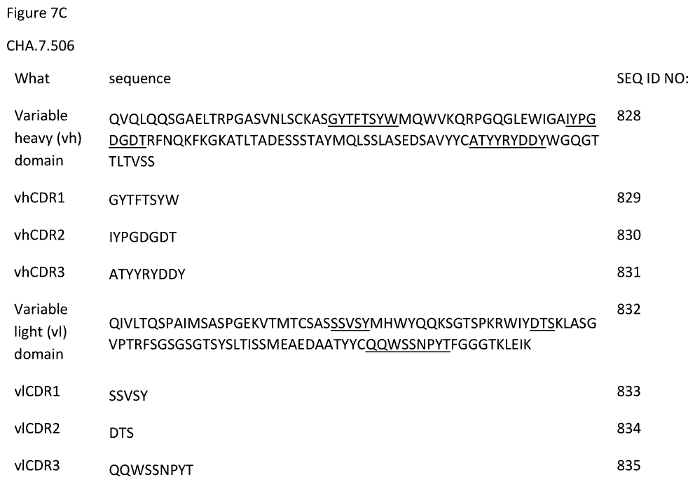

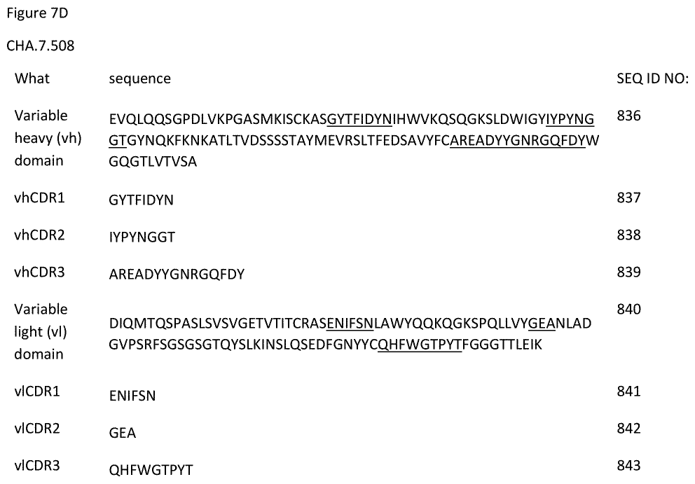

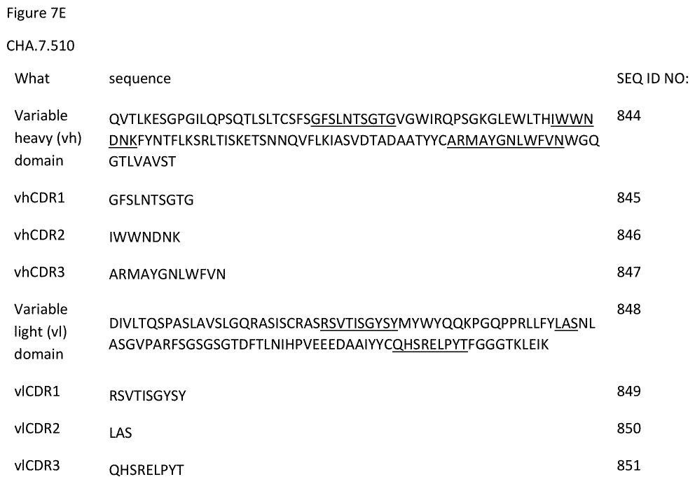

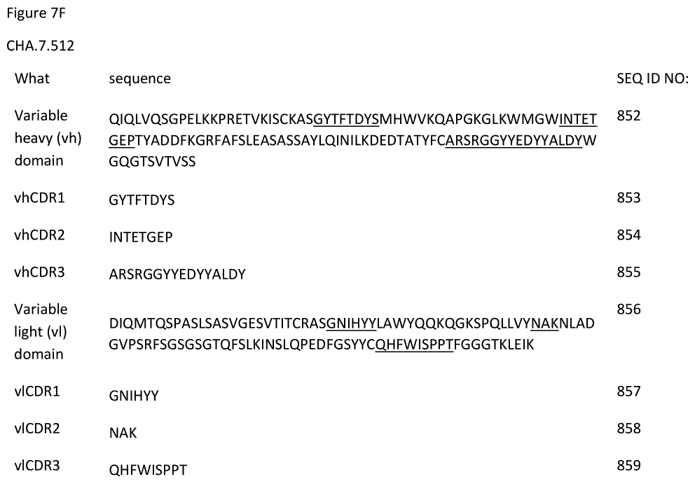

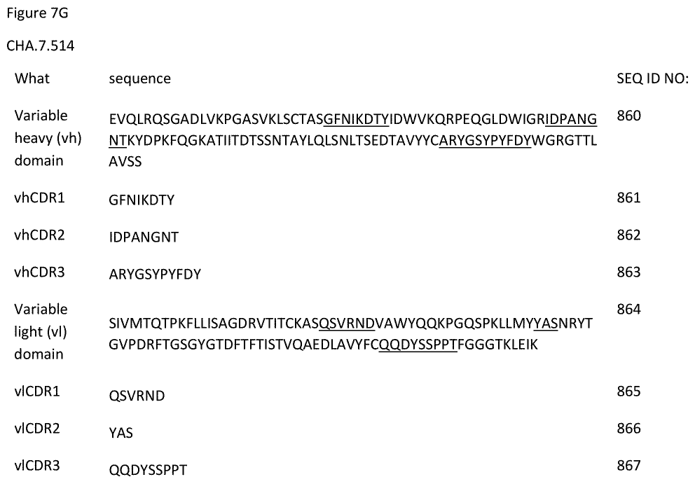

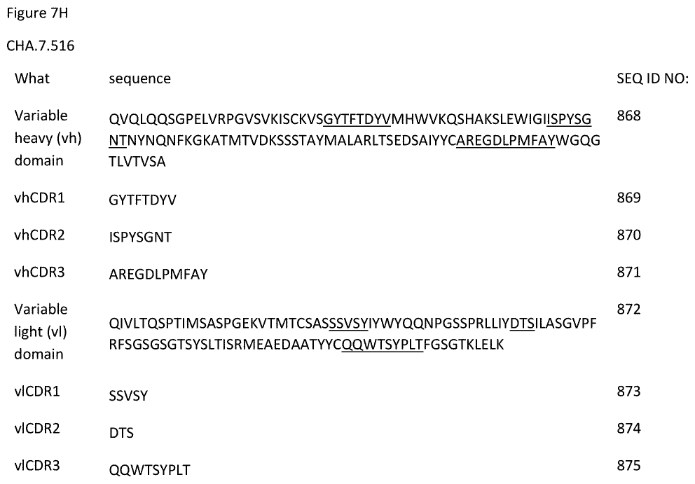

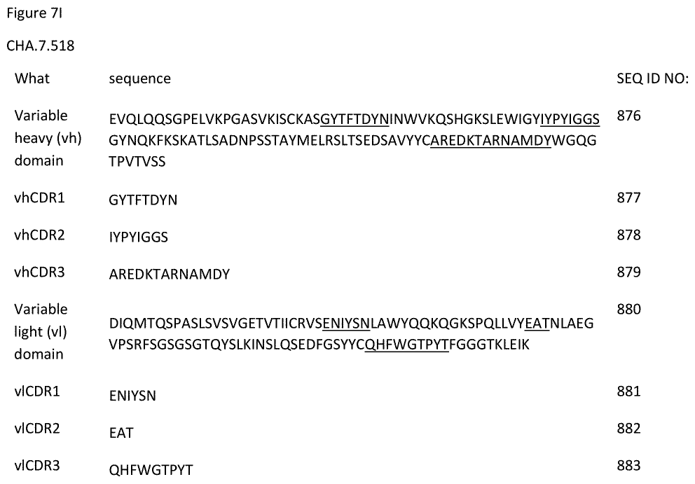

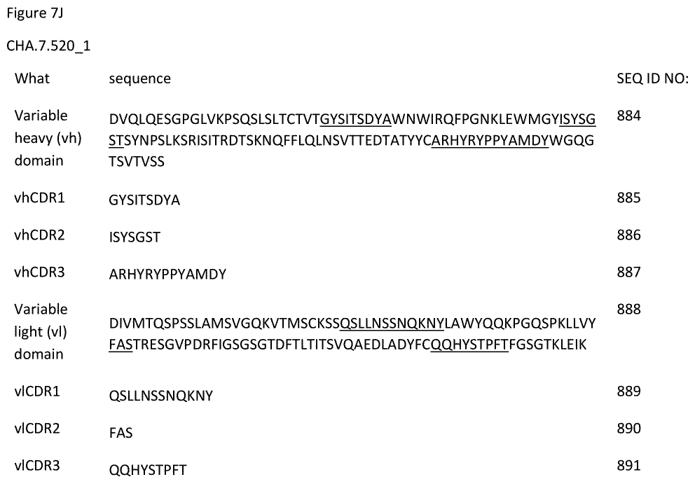

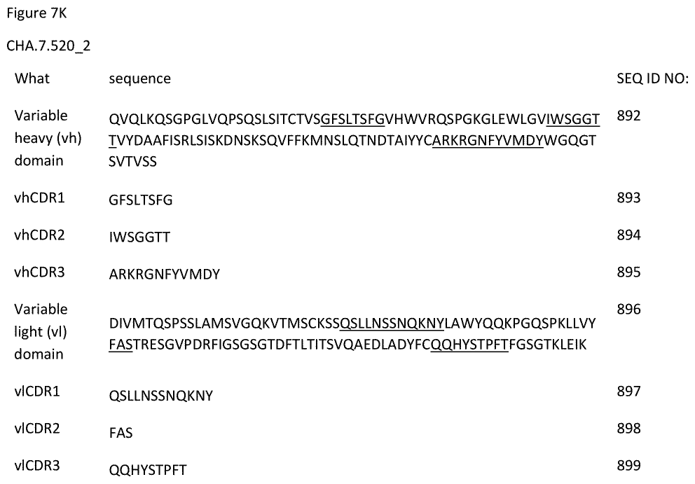

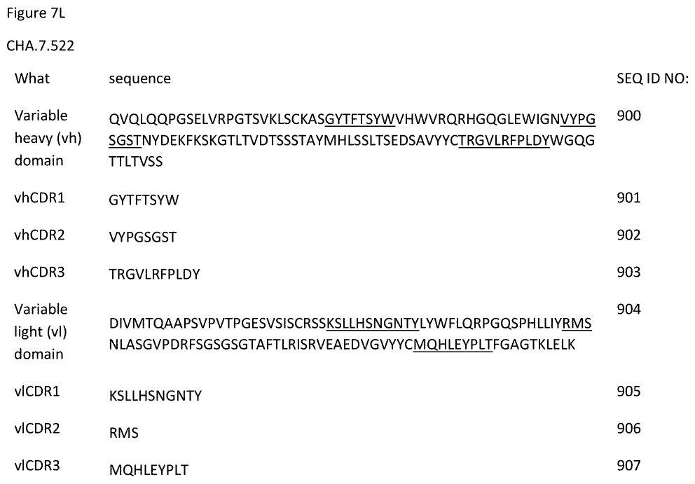

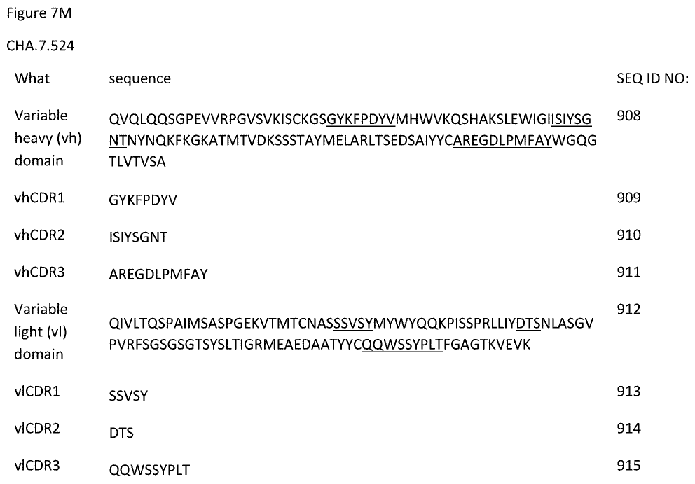

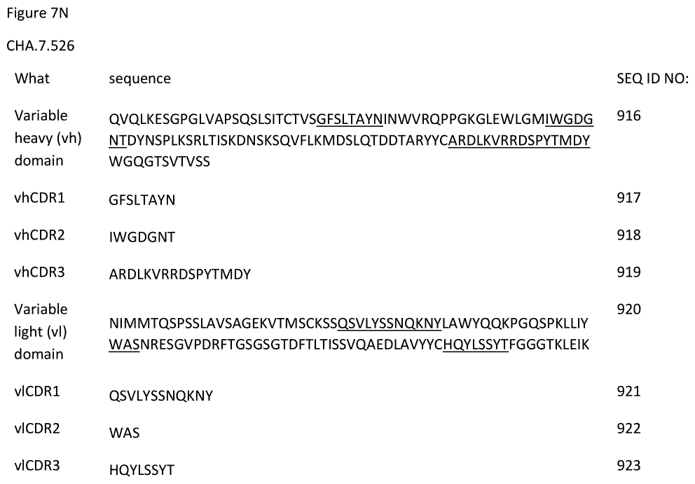

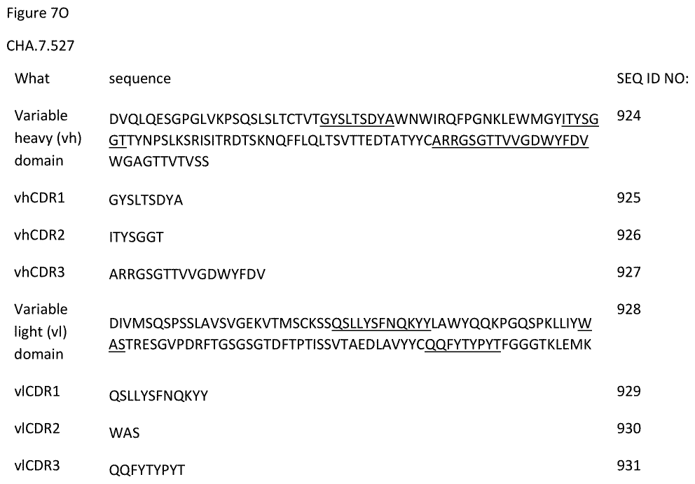

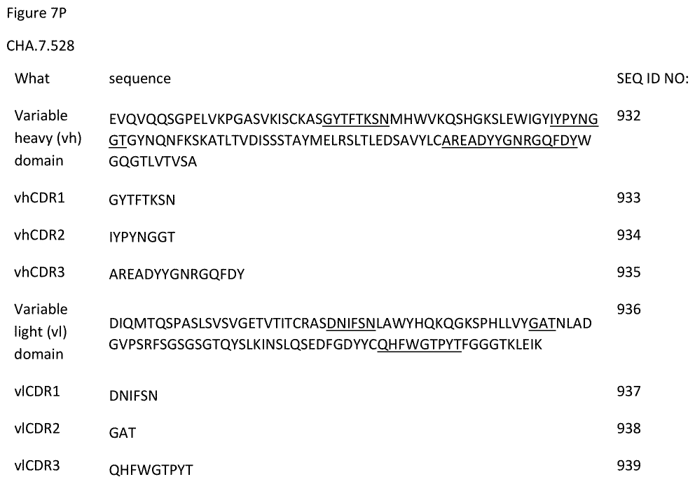

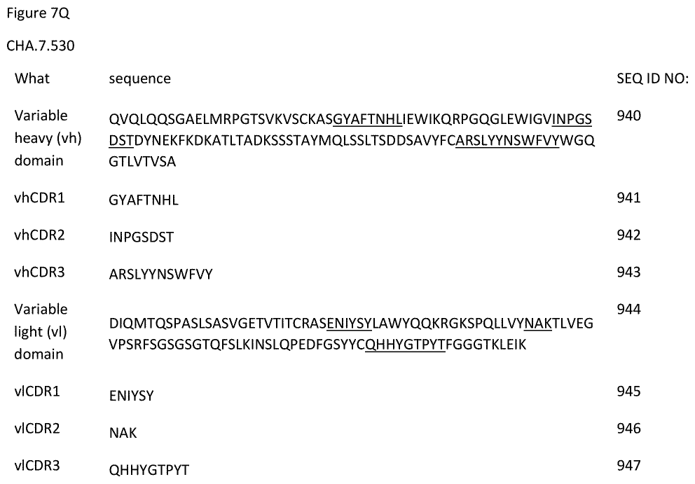

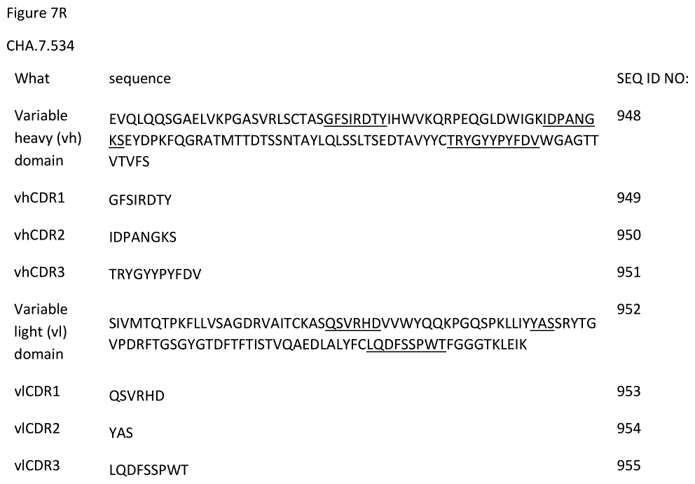

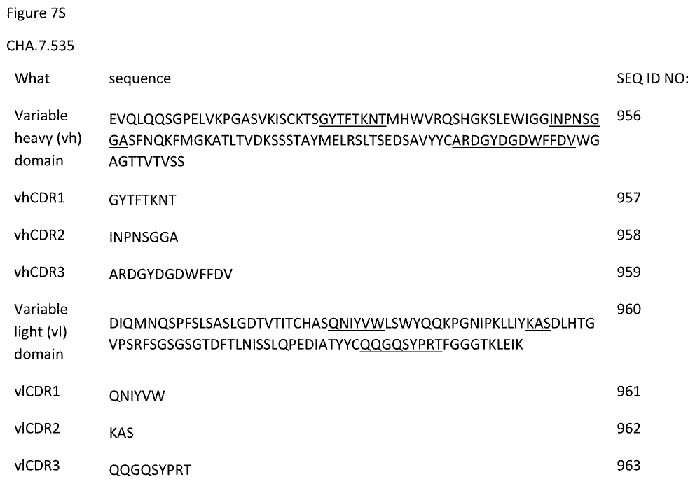

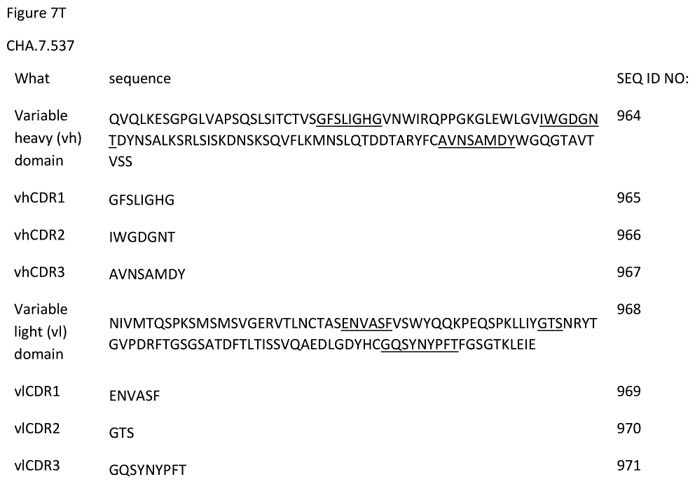

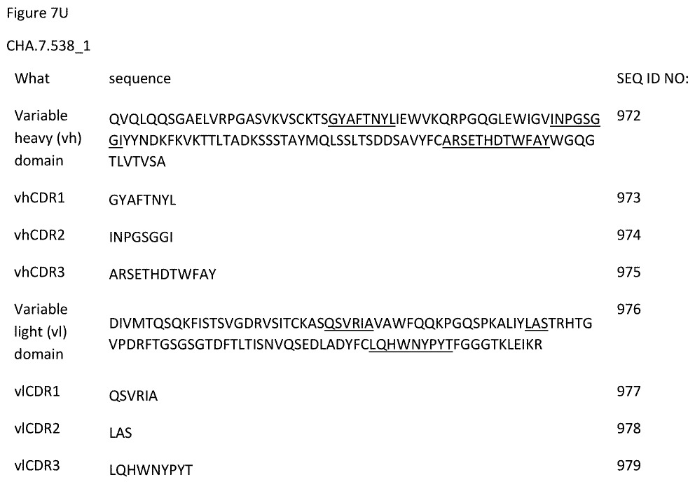

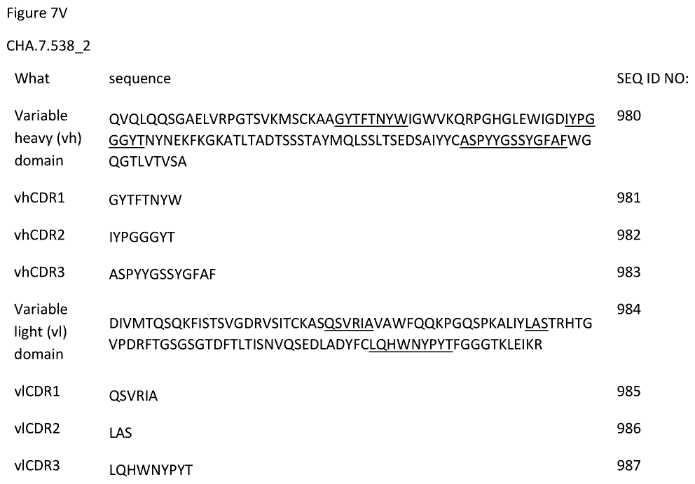

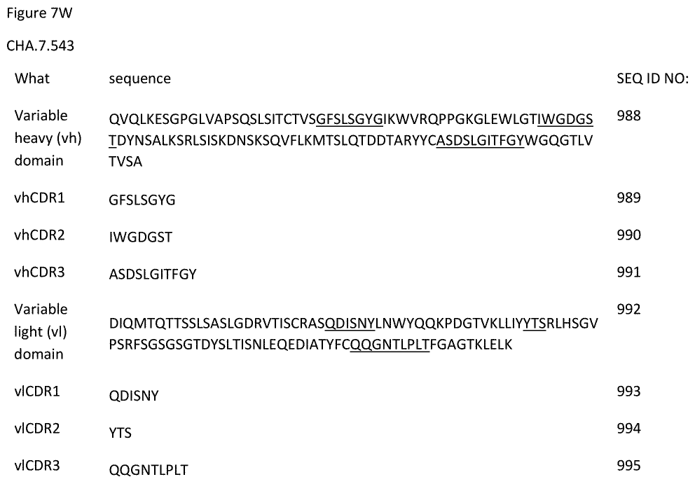

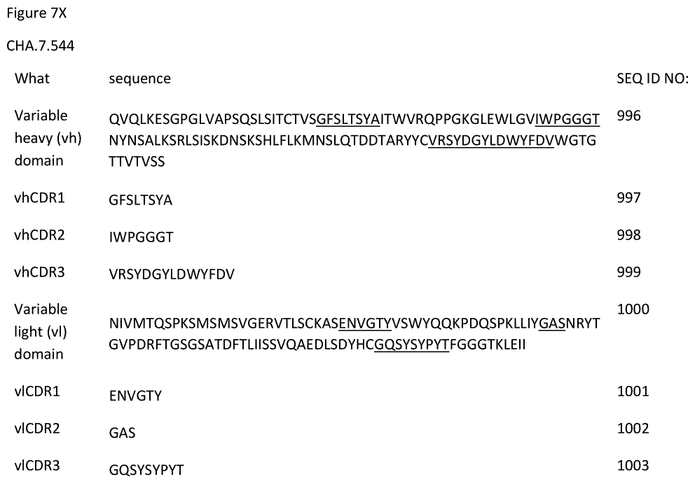

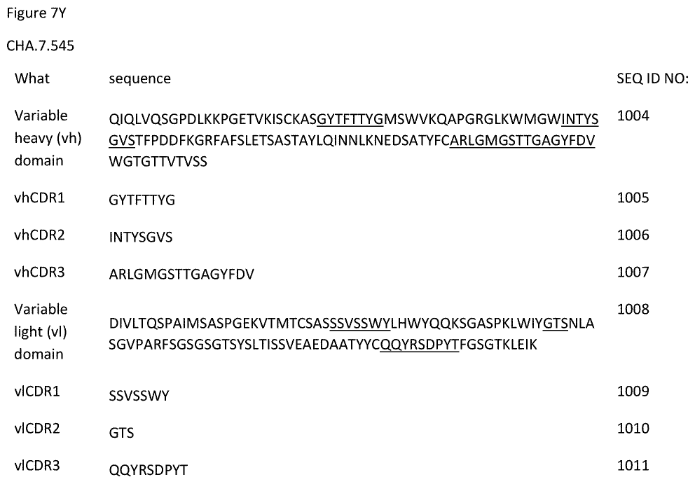

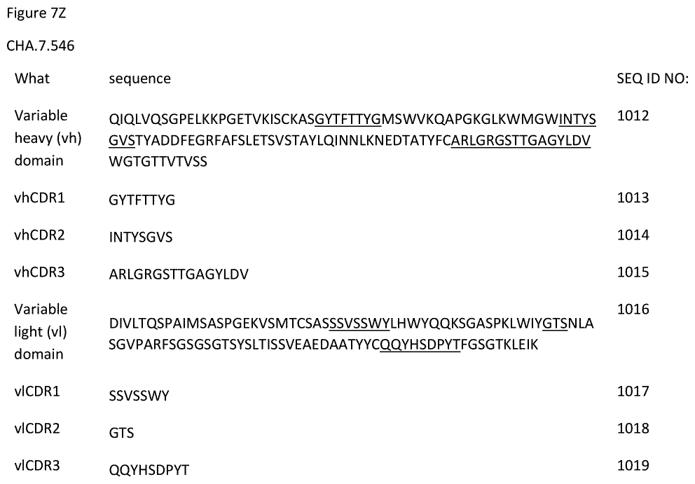

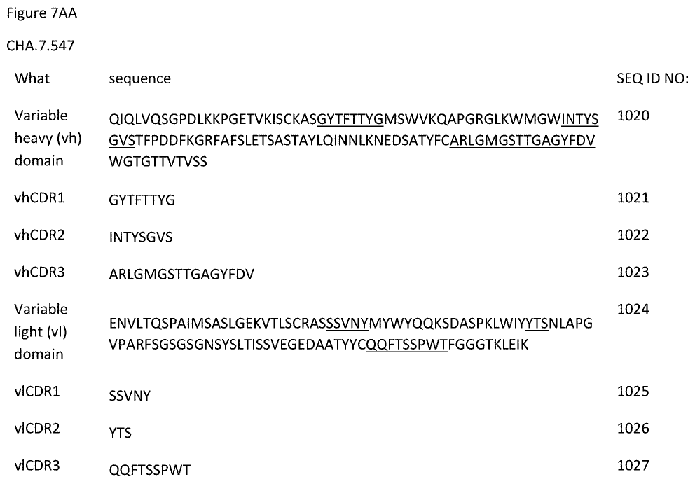

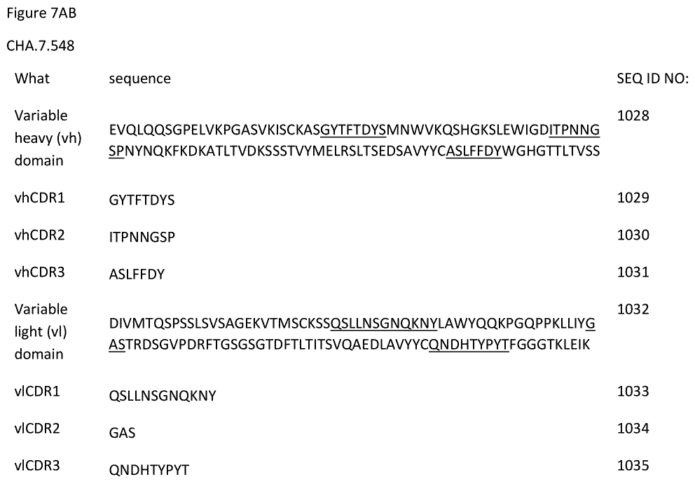

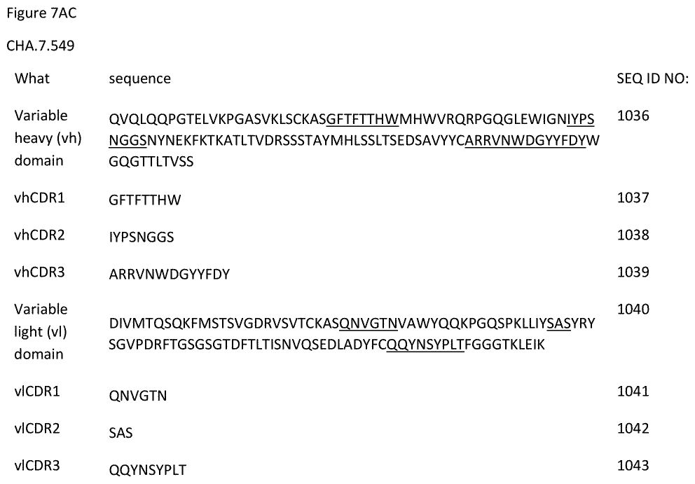

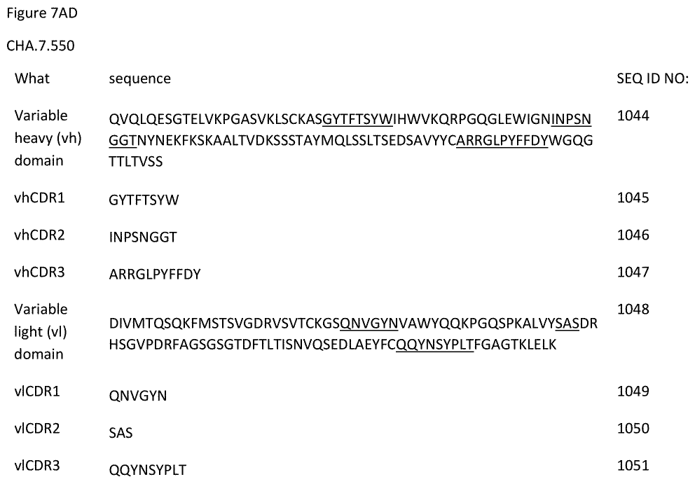

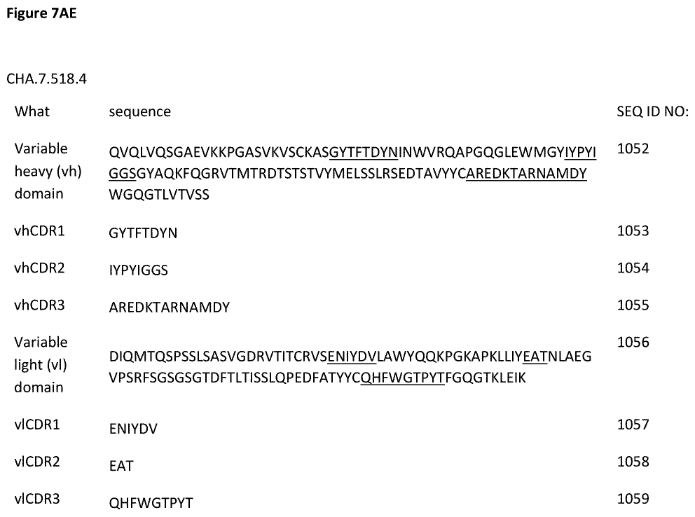

By “linker” is generally meant a peptide linker that is used in the context of an scFv or a bispecific antibody as described herein. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n, (GGGGS)n, and (GGGS)n, where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers, that is may find use as linkers. In some embodiments, linkers are selected from those listed in the Tables 1 and 2 below, (see, also, FIG. 7 from U.S. Pat. No. 9,650,446.)

TABLE 1
Positive charged scFv linkers

| | | | | SEQ |
|---|---|---|---|---|
| | | | | ID |
| Name | Sequence | Length | Charge | NO: |
| Gly-Ser 15 | GGGGSGGGGSGGGGS | 15 | 0 | 3632 |
| Whitlow | GSTSGSGKPGSGEGSTKG | 18 | +1 | 3633 |
| linker | | | | |
| 6paxk_1 | IRPRAIGGSKPRVA | 14 | +4 | 3634 |
| (+A) | | | | |
| +B | GKGGSGKGGSGKGGS | 15 | +3 | 3635 |
| +C | GGKGSGGKGSGGKGS | 15 | +3 | 3636 |
| +D | GGGKSGGGKSGGGKS | 15 | +3 | 3637 |
| +E | GKGKSGKGKSGKGKS | 15 | +6 | 3638 |
| +F | GGGKSGGKGSGKGGS | 15 | +3 | 3639 |
| +G | GKPGSGKPGSGKPGS | 15 | +3 | 3640 |
| +H | GKPGSGKPGSGKPGSGKPGS | 20 | +4 | 3641 |
| +I | GKGKSGKGKSGKGKSGKGKS | 20 | +8 | 3642 |

TABLE 2
Negative charged scFv linkers

| | | | | SEQ |
|---|---|---|---|---|
| | | | | ID |
| Name | Sequence | Length | Charge | NO: |
| Gly-Ser 15 | GGGGSGGGGSGGGGSGGGGS | 20 | 0 | 3643 |
| 3hsc2 (−A) | STAGEITHLGGEDFD | 14 | −4 | 3644 |
| −B | GEGGSGEGGSGEGGS | 15 | −3 | 3645 |
| −C | GGEGSGGEGSGGEGS | 15 | −3 | 3646 |
| −D | GGGESGGGESGGGES | 15 | −3 | 3647 |
| −E | GEGESGEGESGEGFS | 1.5 | −6 | 3648 |
| −F | GGGESGGEGSGEGGS | 15 | −3 | 3649 |
| −G | GEGESGEGESGEGESGEGES | 20 | −8 | 3650 |

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification. Similarly, because IgG1 has a proline at position 241 and IgG4 has a serine there, an IgG4 molecule with a S241P is considered an IgG subclass modification. Note that subclass modifications are considered amino acid substitutions herein.

By “non-naturally occurring modification” as used herein is meant an amino acid modification that is not isotypic. For example, because none of the IgGs comprise AN asparagine at position 297, the substitution N297A in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRls, FcγRlls, FcγRllls, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below.

By “Fc” or “Fc region” or “Fc domain” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

By “heavy constant region” herein is meant the CH1-hinge-CH2-CH3 portion of an antibody.

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. In the present case, one target antigen of interest herein is TIGIT, usually human TIGIT and optionally cyno TIGIT, as defined below. Another target antigen of interest is PVRIG, usually human PVRIG and optionally cyno PVRIG, as defined below.

By “target cell” as used herein is meant a cell that expresses a target antigen.

By “variable region” as used herein is meant the region of an immunoglobulin that comprises one or more Ig domains substantially encoded by any of the Vκ (V.kappa), Vλ (V.lamda), and/or VH genes that make up the kappa, lambda, and heavy chain immunoglobulin genetic loci respectively.

By “wild type or WT” herein is meant an amino acid sequence or a nucleotide sequence that is found in nature, including allelic variations. A WT protein has an amino acid sequence or a nucleotide sequence that has not been intentionally modified.

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁹ M, at least about 10⁻¹⁰ M, at least about 10⁻¹¹ M, at least about 10⁻¹² M, at least about 10⁻¹³ M, at least about 10⁻¹⁴ M, at least about 10⁻¹⁵ M, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using surface plasmon resonance (e.g. Biacore assay) and flow cytometry with antigen-expressing cells.

### C. Sequences

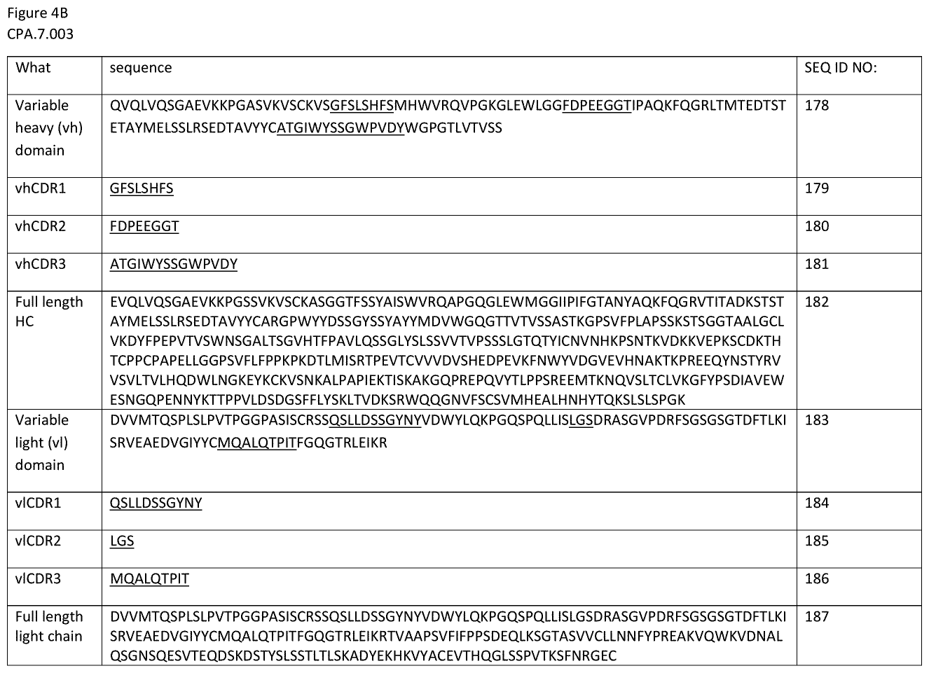

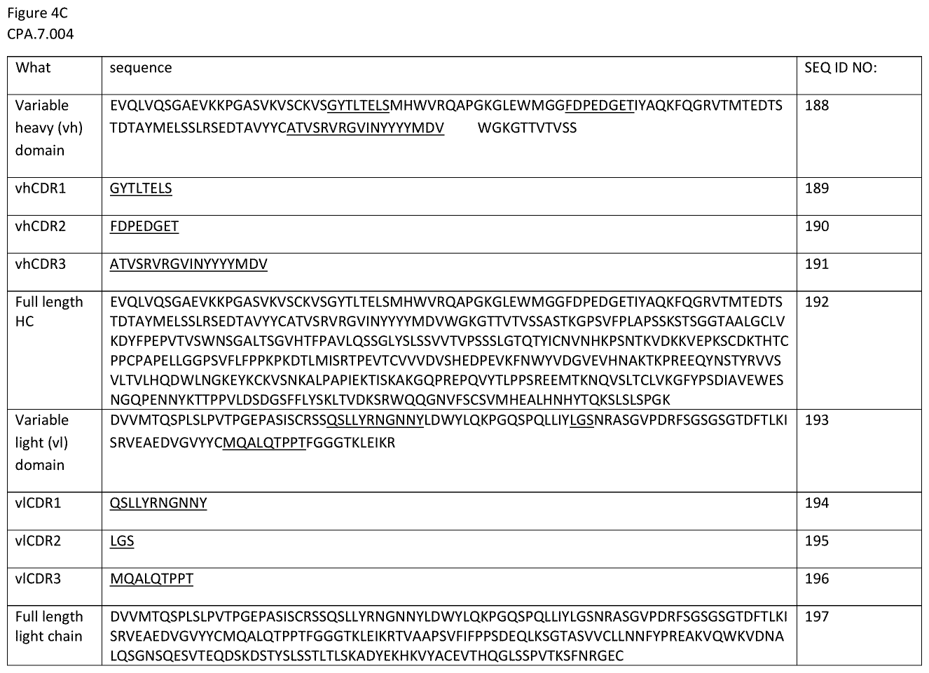

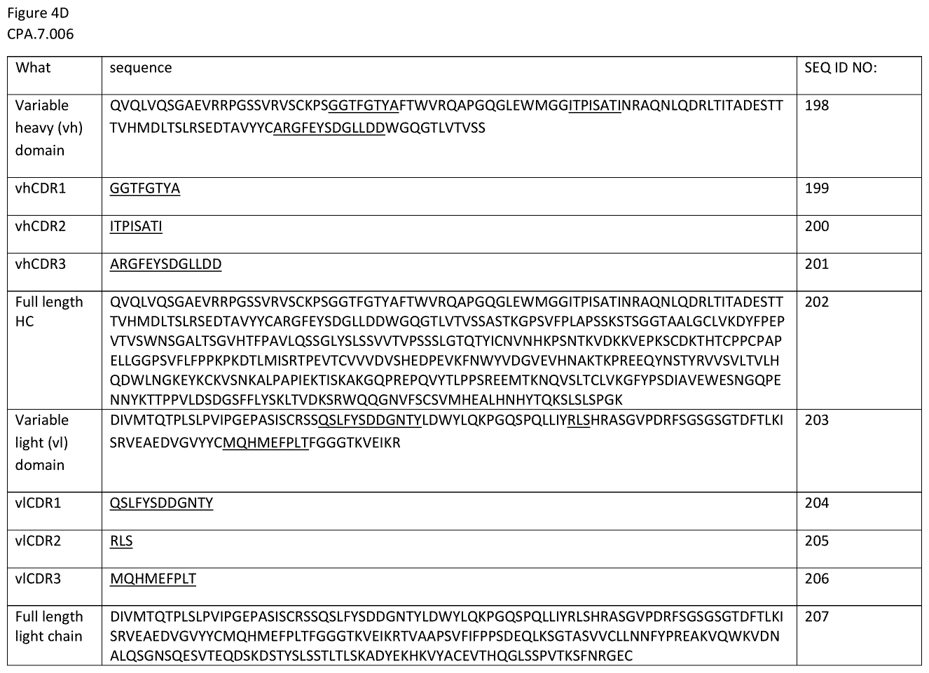

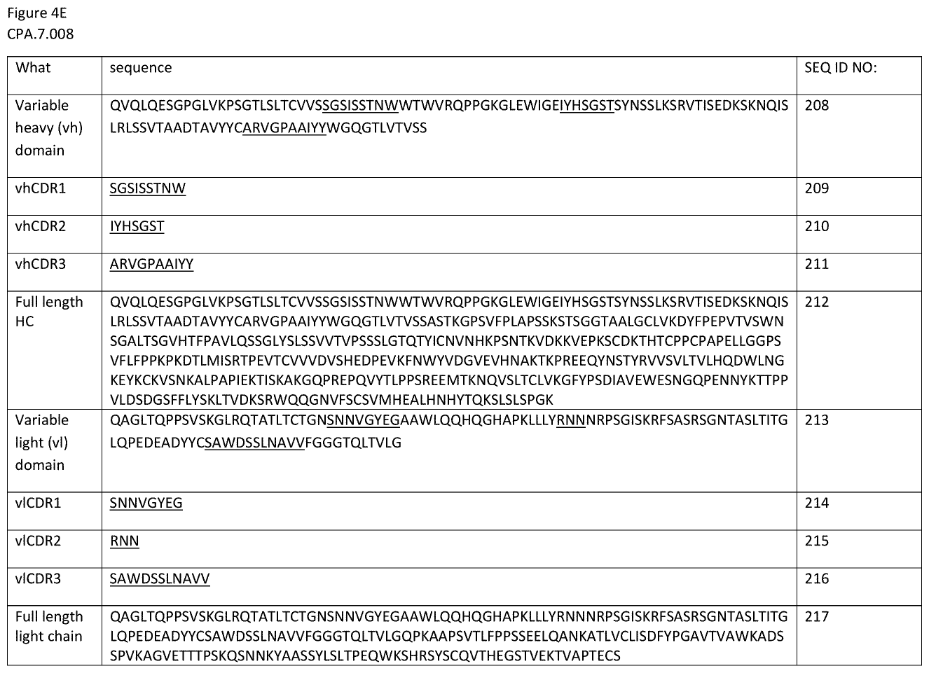

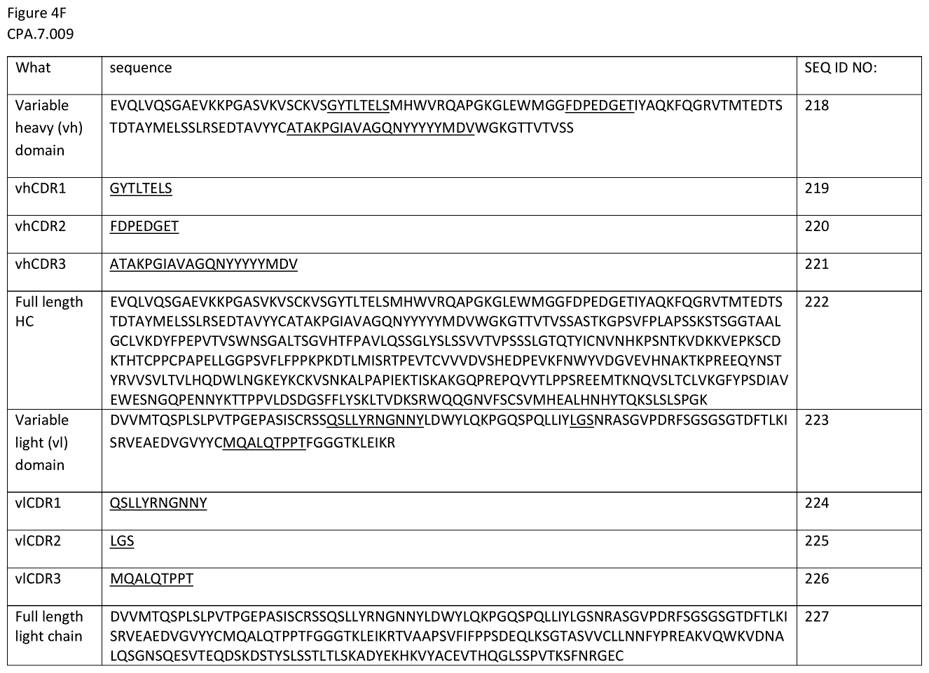

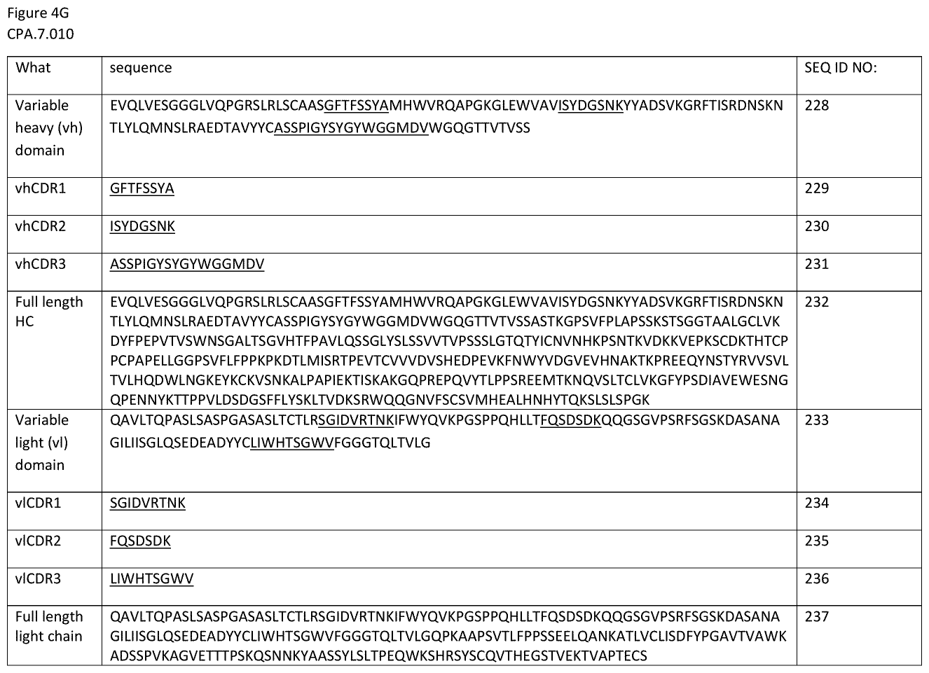

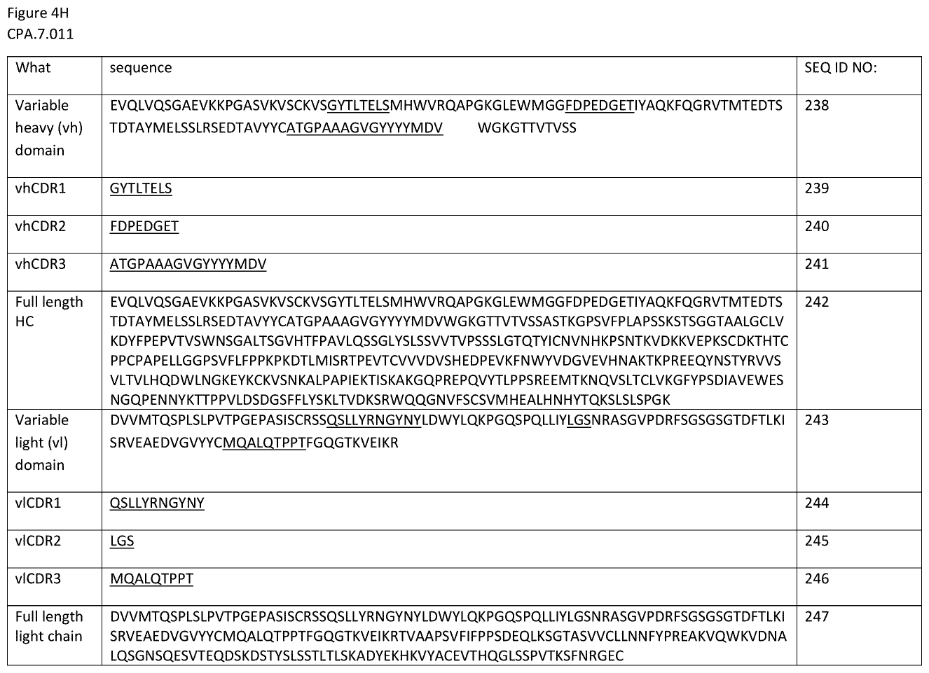

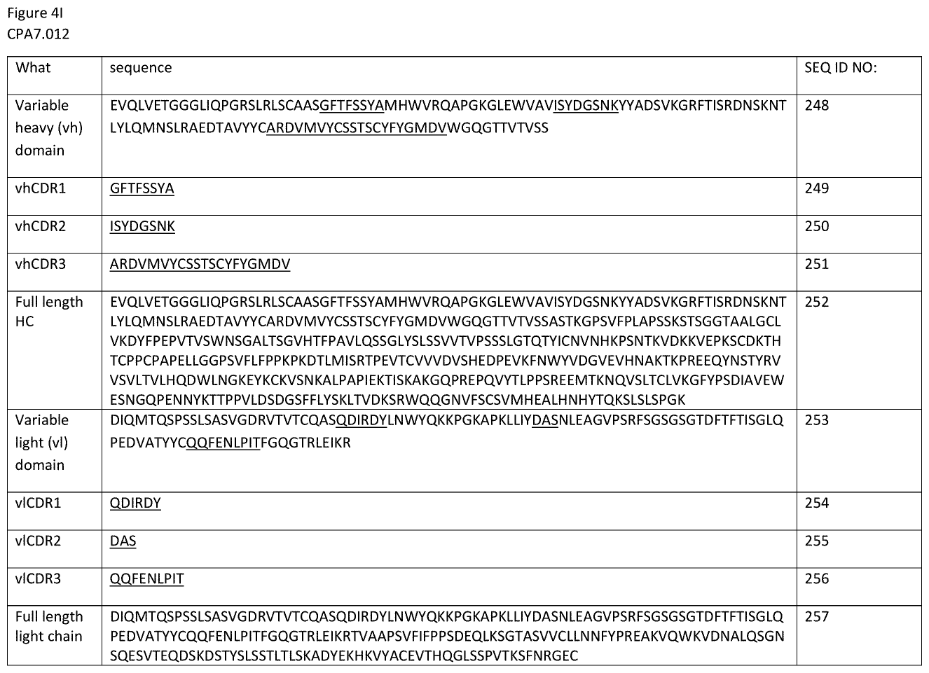

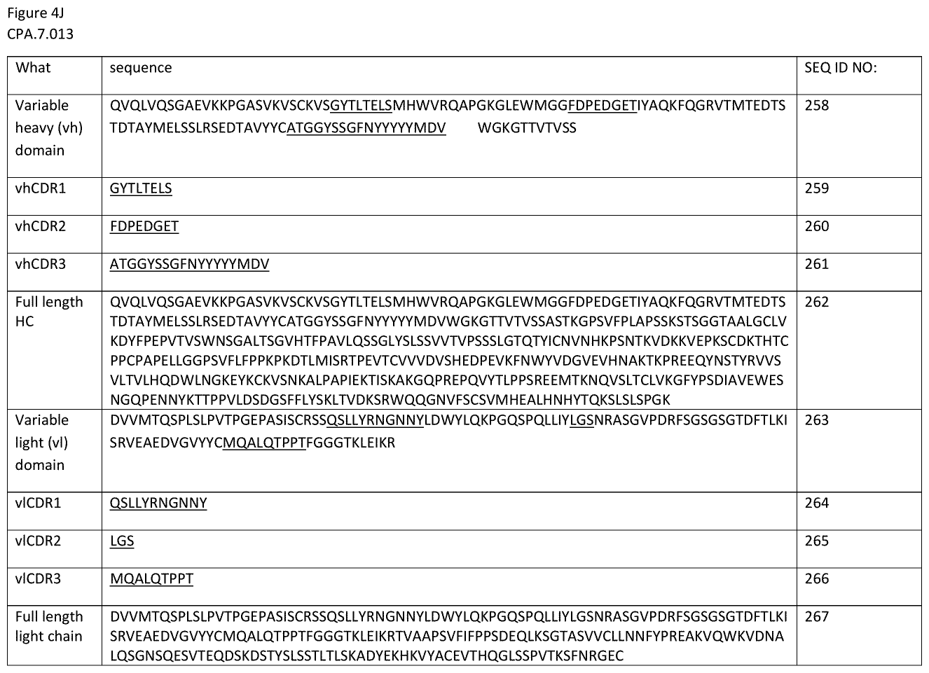

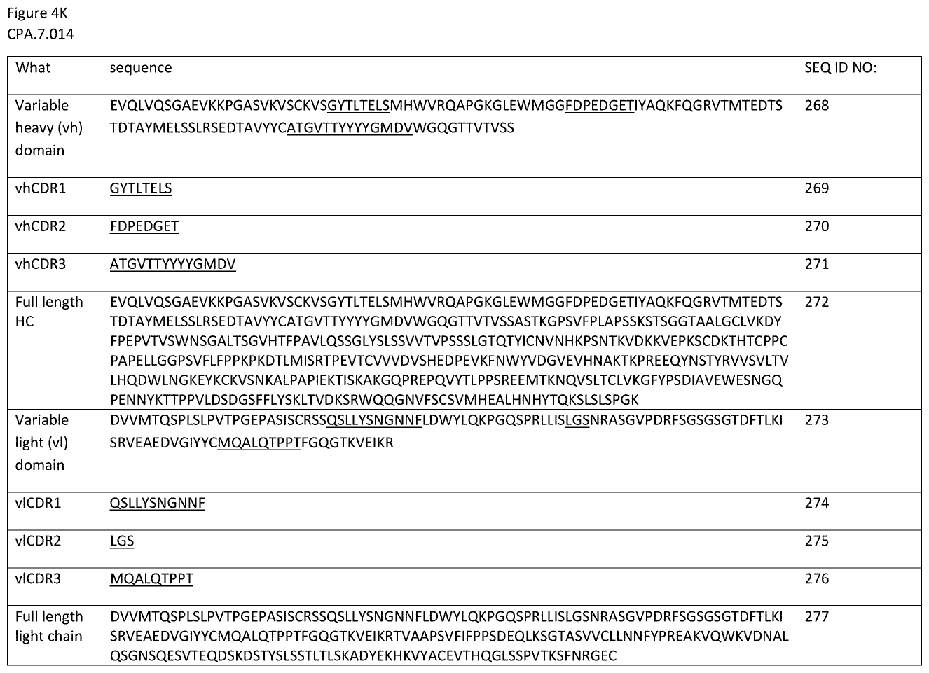

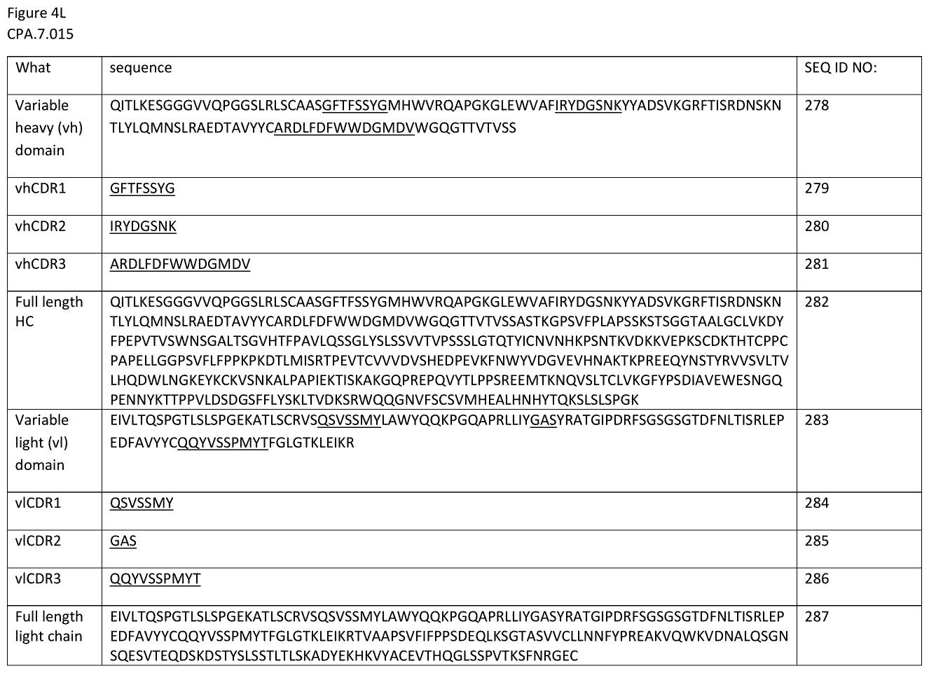

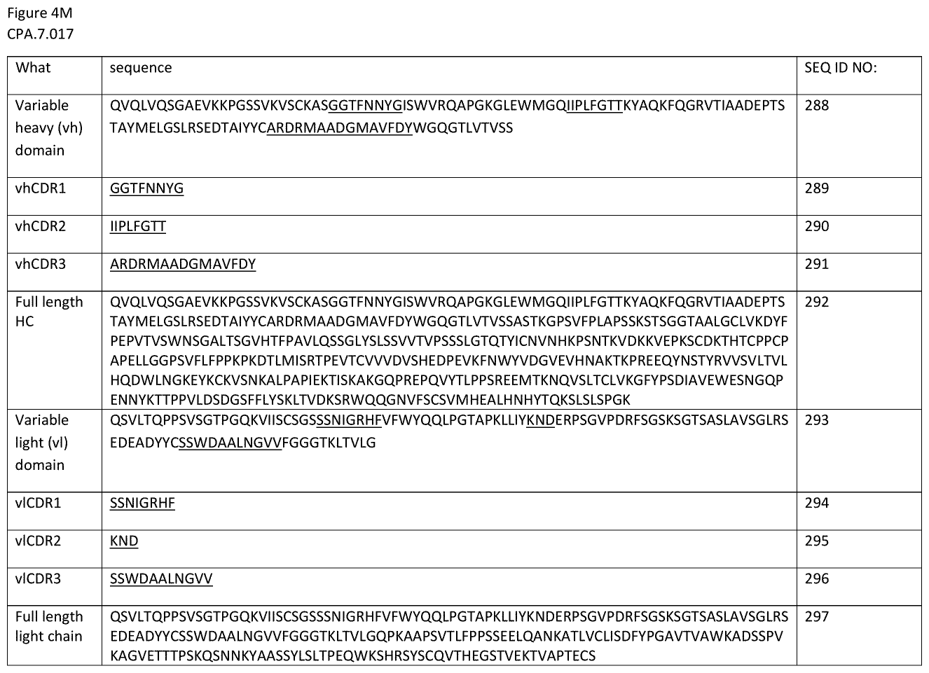

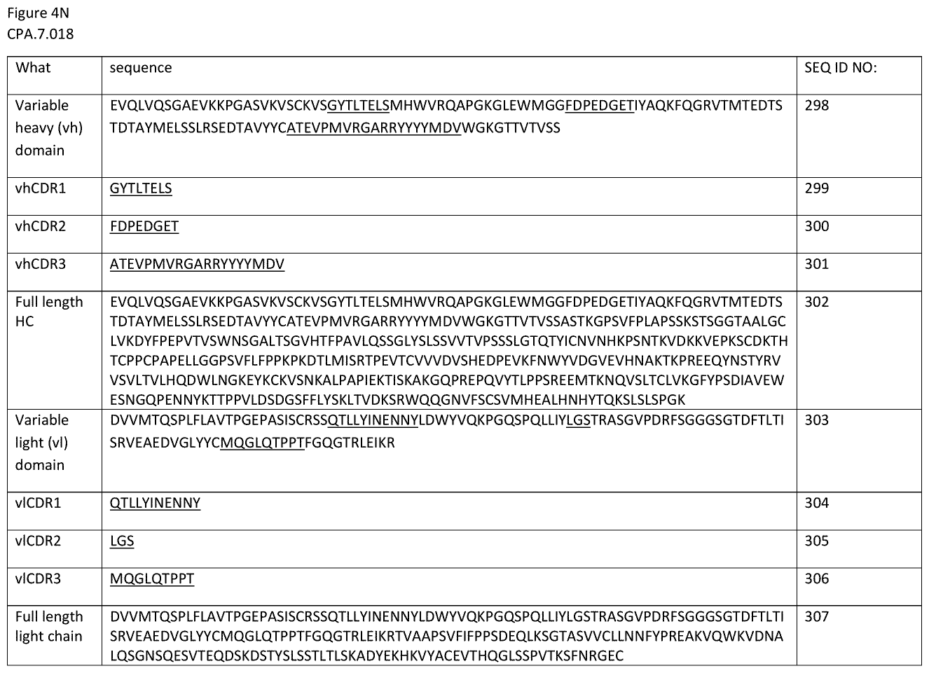

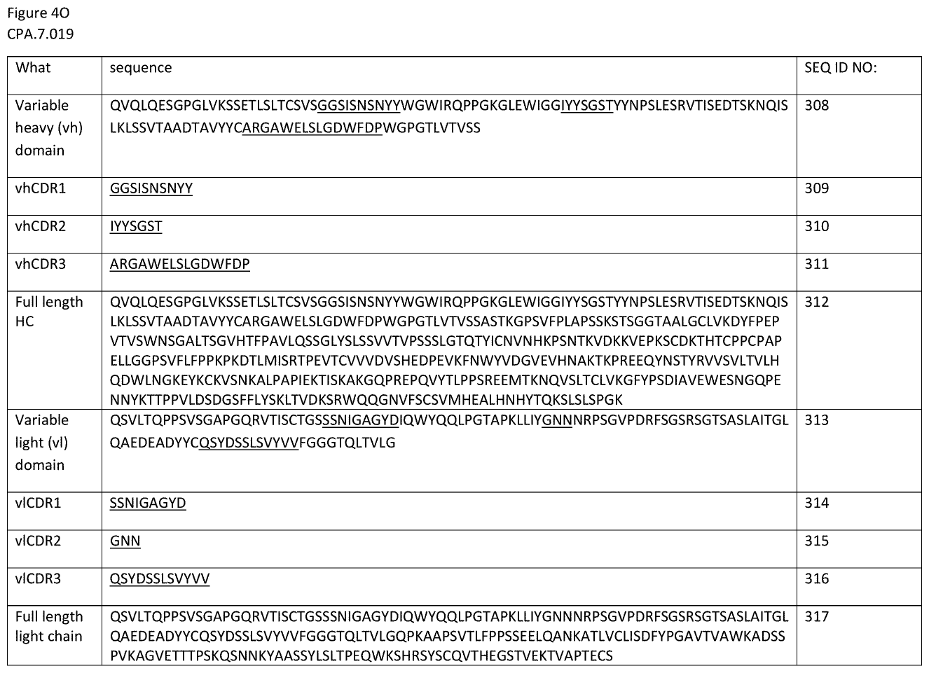

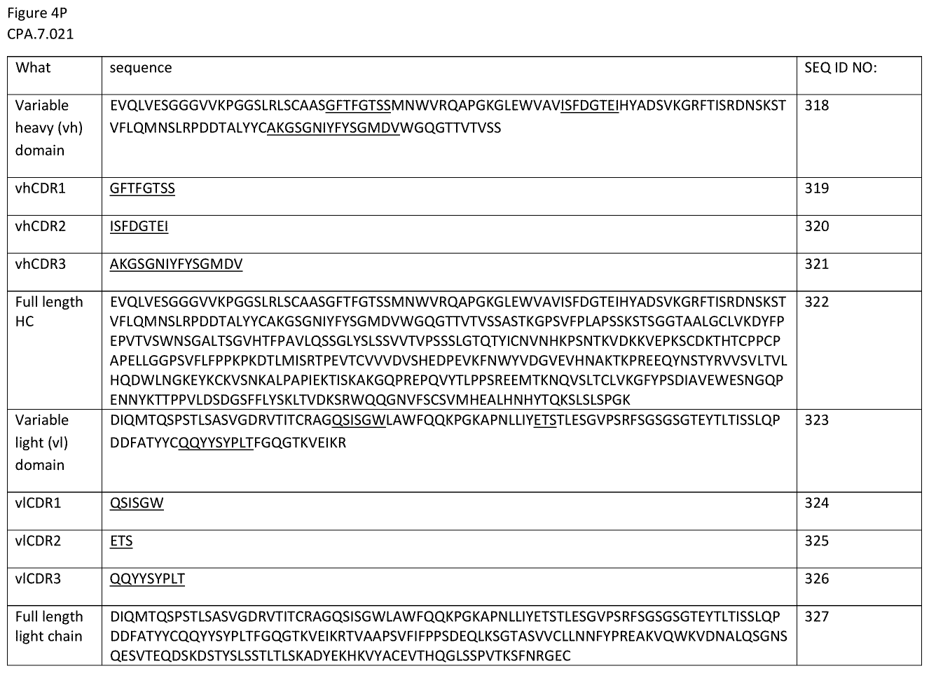

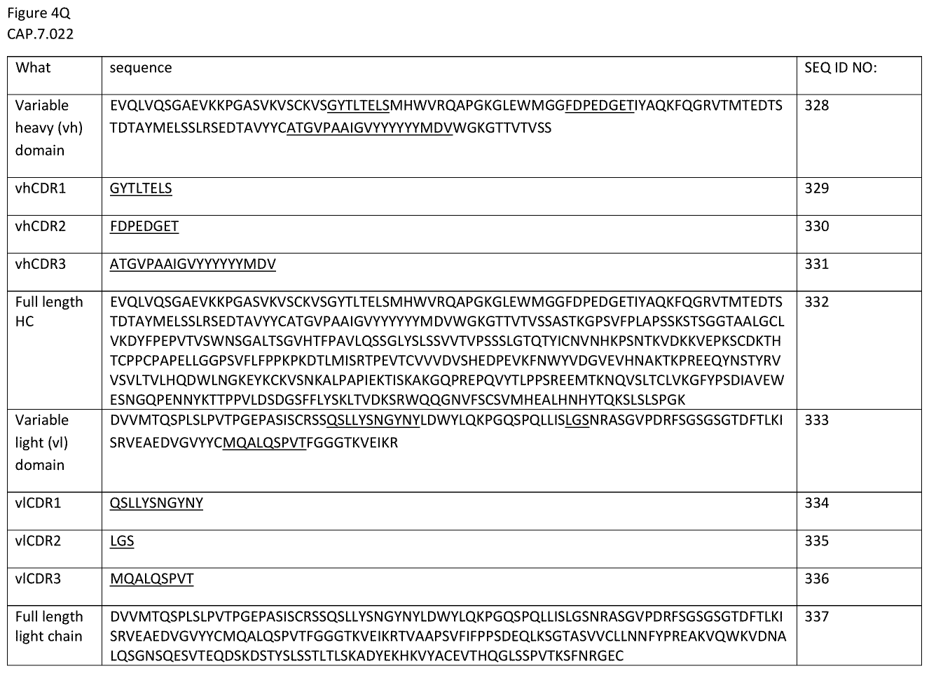

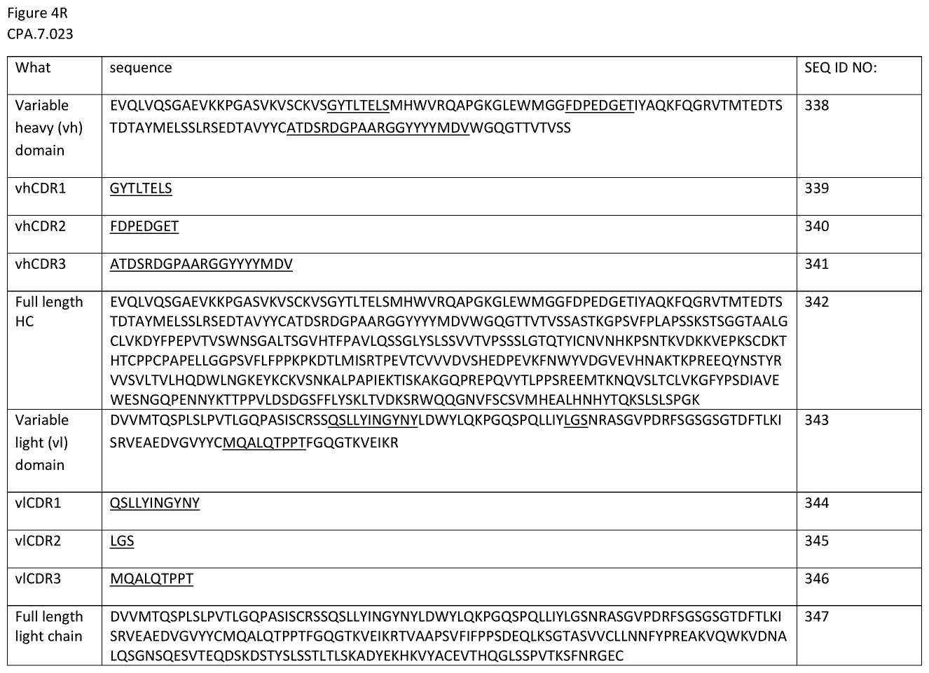

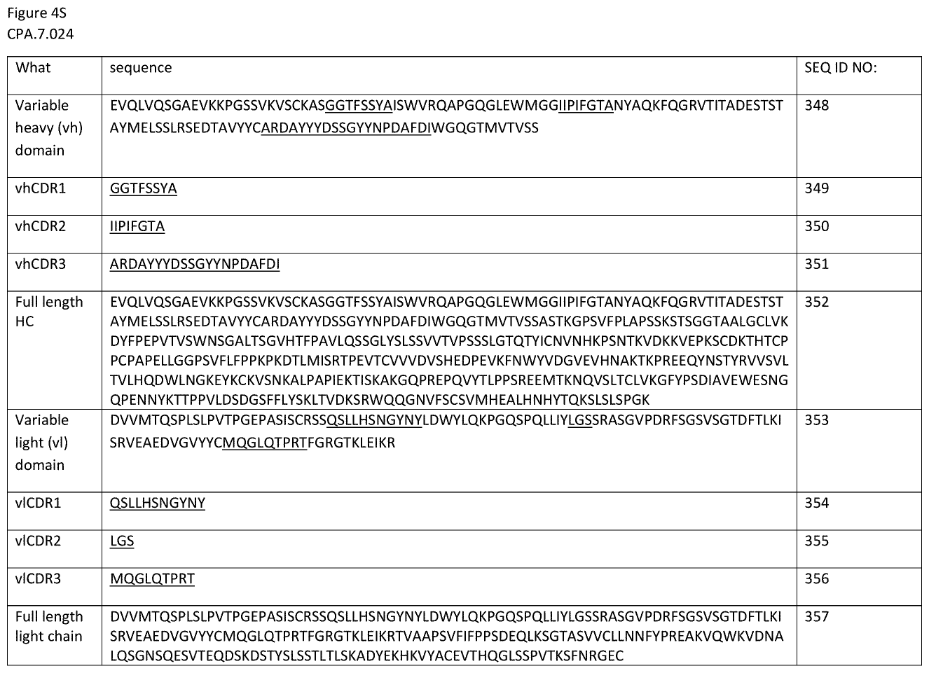

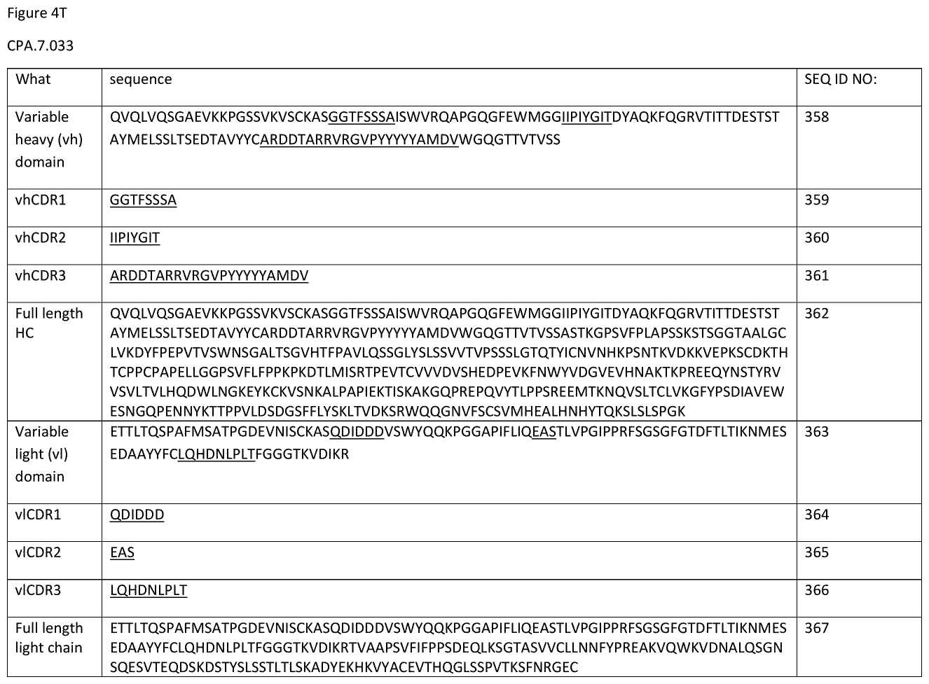

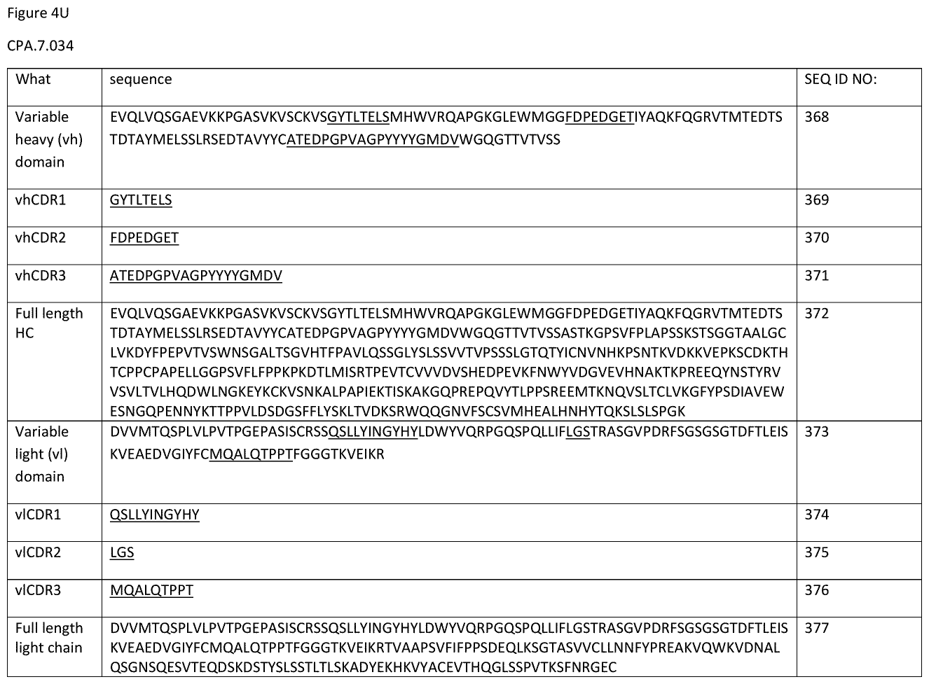

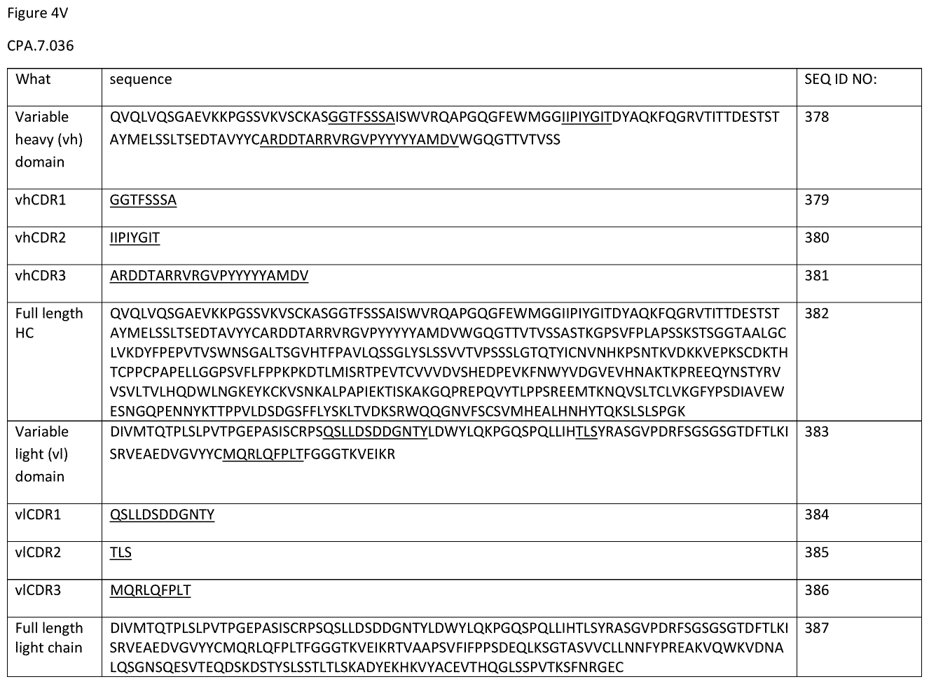

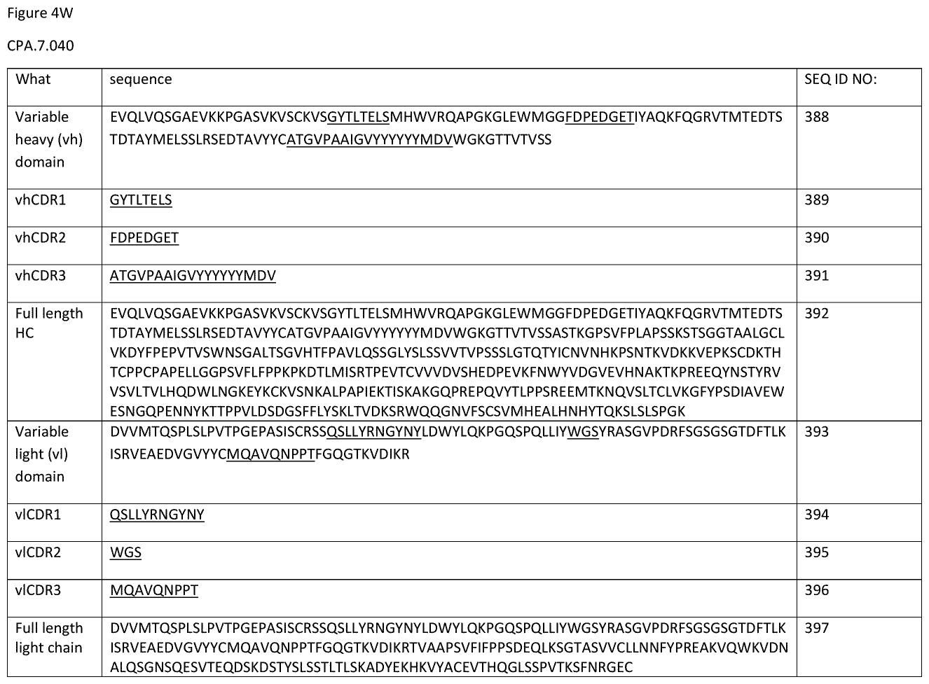

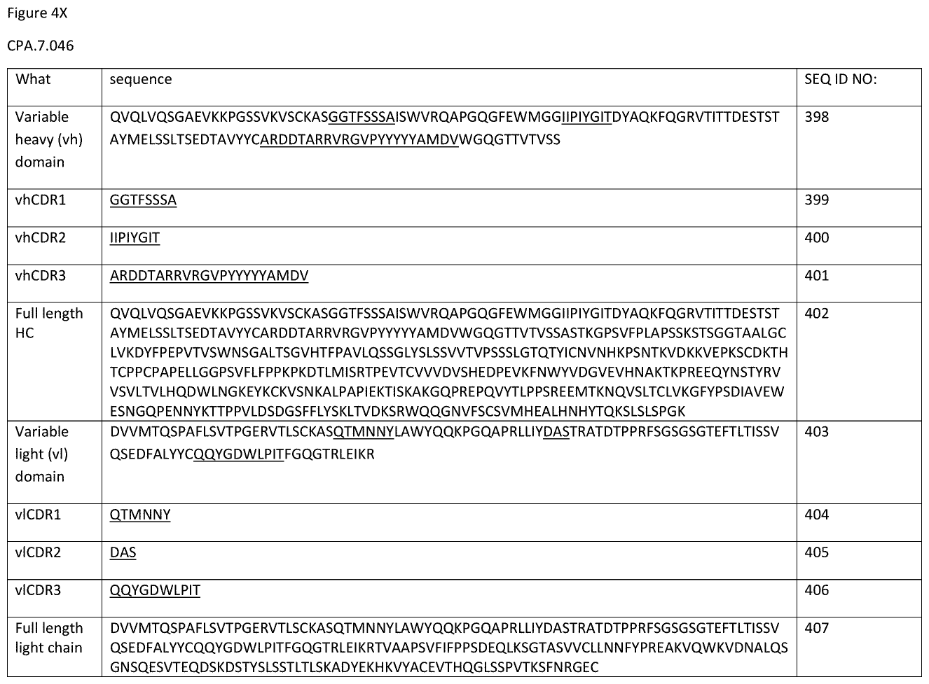

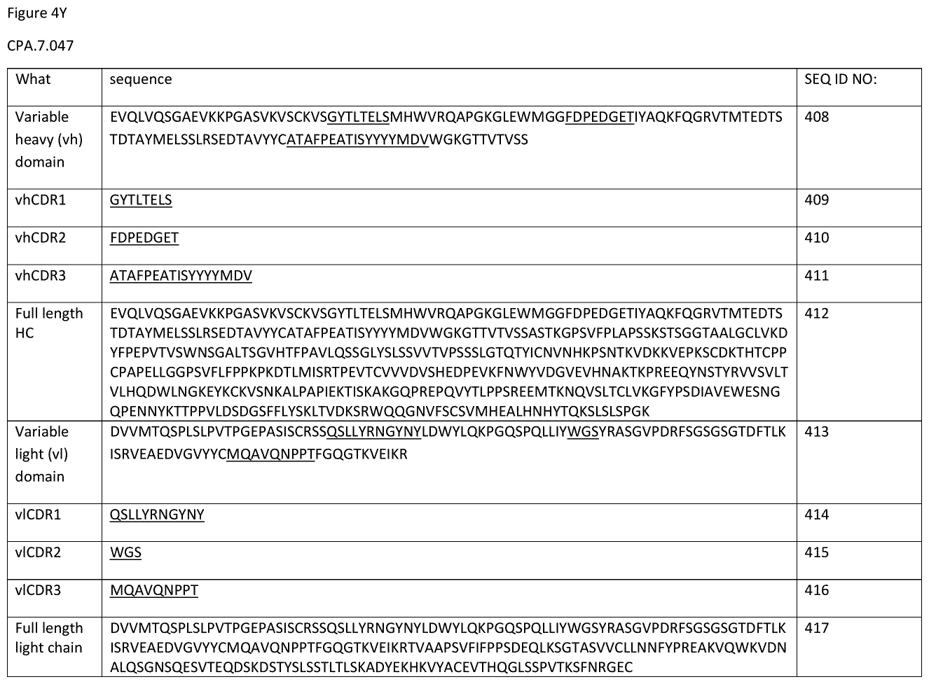

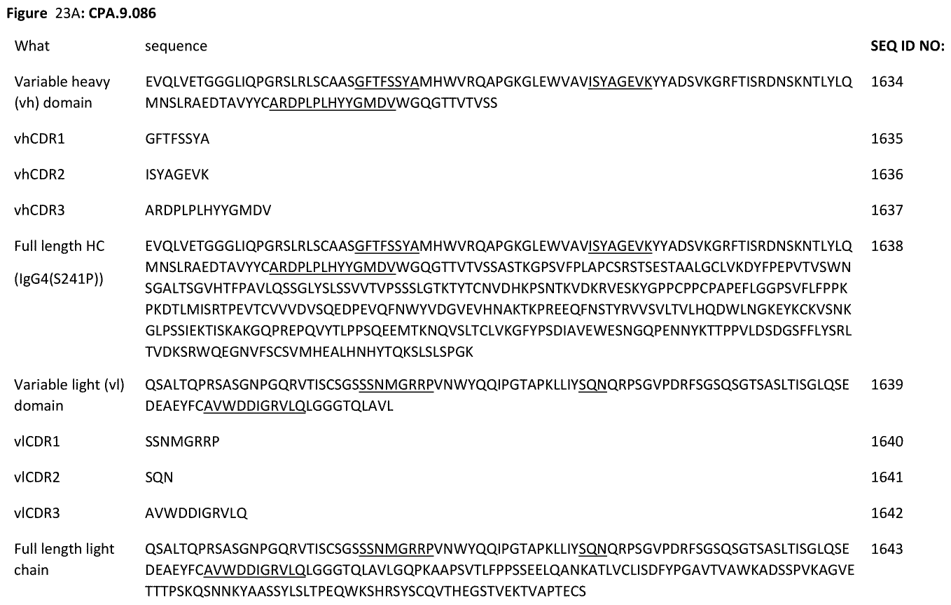

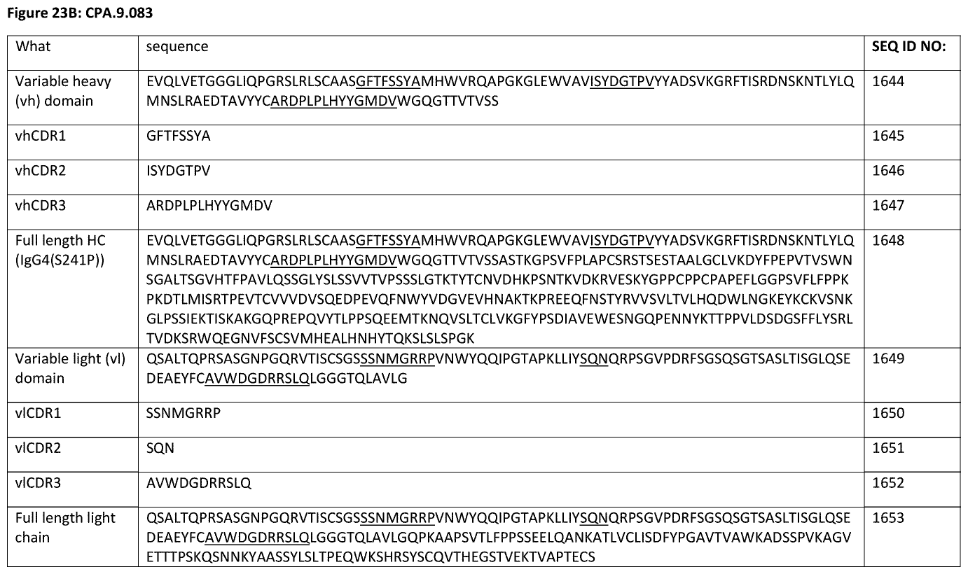

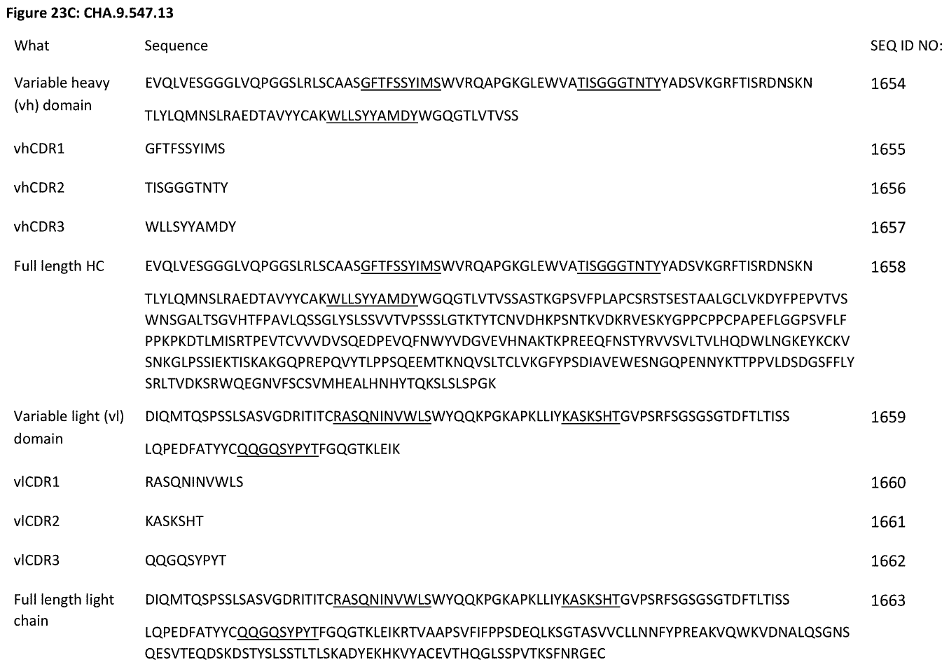

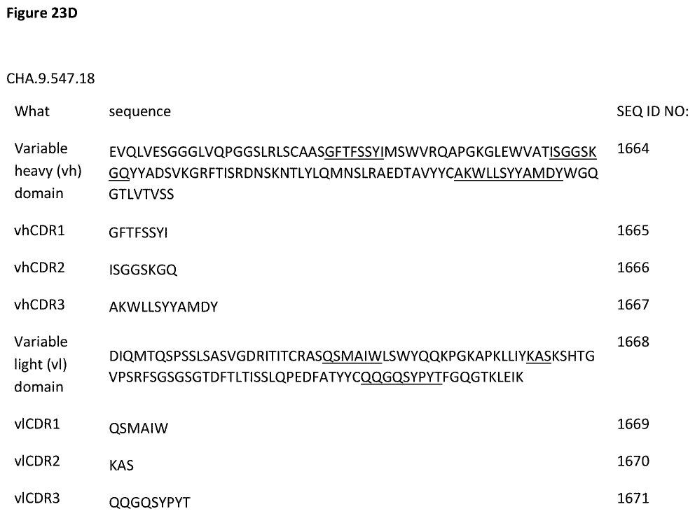

The sequence listing provides a number of sequences based on the Format of FIG. 23A-23D; reference is made to FIG. 4 of U.S. Ser. No. 62/513,916 (hereby expressly incorporated by reference) as a guide to the labeling of the sequences. The variable heavy domain is labeled with the identifier (e.g., “CPA.9.086”), with the next sequence following the format of FIG. 23A-23D of the present specification (identical to the format of FIG. 4, referenced above), in that the next sequence identifier is to the vhCDR1, the next to vhCDR2, with vhCDR3, the full length heavy chain, the variable light domain, vlCDR1, vlCDR2, vlCDR3 and the full length light chain. Thus an individual antibody has 10 associated sequence identifiers). Included in the sequence listing are the sequences of BM26 mouse IgG1 (BM26-M1) (WO2016/028656A1, Clone 3106) and BM29 mouse IgG1 (BM29-M1) (US2016/0176963A1, Clone 22G2). Unless noted, the full length HC sequences of the PVRIG and/or TIGIT antibodies are in the H4(S241P) format.

### D. PVRIG Proteins

The present invention provides anti-PVRIG and/or anti-PVRIG/anti-TIGIT bispecific antibodies that specifically bind to PVRIG proteins and prevent activation by its ligand protein, PVRL2, a human plasma membrane glycoprotein. PVRIG, also called Poliovirus Receptor Related Immunoglobulin Domain Containing Protein, Q6DKI7 or C7orf15, relates to amino acid and nucleic acid sequences shown in RefSeq accession identifier NP_076975, shown in FIG. 1. The sequence of human Poliovirus receptor-related 2 protein (PVLR2, also known as nectin-2, CD112 or herpesvirus entry mediator B, (HVEB)), the binding partner of PVRIG (as shown in Example 5 of US Publication 2016/0244521), is shown in FIG. 2. The anti-PVRIG/anti-TIGIT bispecific antibodies of the invention are specific for the PVRIG extracellular domain such that the binding of PVRIG and PVLR2 is blocked.

PVRIG is a transmembrane domain protein of 326 amino acids in length, with a signal peptide (spanning from amino acid 1 to 40), an extracellular domain (spanning from amino acid 41 to 171), a transmembrane domain (spanning from amino acid 172 to 190) and a cytoplasmic domain (spanning from amino acid 191 to 326). There are two methionines that can be start codons, but the mature proteins are identical.

Accordingly, as used herein, the term “PVRIG” or “PVRIG protein” or “PVRIG polypeptide” may optionally include any such protein, or variants, conjugates, or fragments thereof, including but not limited to known or wild type PVRIG, as described herein, as well as any naturally occurring splice variants, amino acid variants or isoforms, and in particular the ECD fragment of PVRIG.

As noted herein and more fully described below, anti-PVRIG and/or anti-PVRIG/anti-TIGIT bispecific antibodiesthat both bind to PVRIG and prevent activation by PVRL2 (e.g. most commonly by blocking the interaction of PVRIG and PVLR2), are used to enhance T cell and/or NK cell activation and be used in treating diseases such as cancer and pathogen infection.

### E. TIGIT Proteins

The present invention provides anti-TIGIT, and/or anti-PVRIG/anti-TIGIT bispecific antibodiesthat specifically bind to TIGIT proteins and prevent activation by its ligand protein, PVR, poliovirus receptor (aka CD155) a human plasma membrane glycoprotein. TIGIT, or T cell immunoreceptor with Ig and ITIM domains, is a co-inhibiotry receptor protein also known as WUCAM, Vstm3 or Vsig9. TIGIT has an immunoglobulin variable domain, a transmembrane domain, and an immunoreceptor tyrosine-based inhibitory motif (ITIM) and contains signature sequence elements of the PVR protein family. The extracellular domain (ECD) sequences of TIGIT and of PVR are shown in FIG. 22. The antibodies of the invention are specific for the TIGIT ECD such that the binding of TIGIT and PVR is blocked

Accordingly, as used herein, the term “TIGIT” or “TIGIT protein” or “TIGIT polypeptide” may optionally include any such protein, or variants, conjugates, or fragments thereof, including but not limited to known or wild type TIGIT, as described herein, as well as any naturally occurring splice variants, amino acid variants or isoforms, and in particular the ECD fragment of TIGIT.

As noted herein and more fully described below, anti-TIGIT antibodies (including antigen-binding fragments) that both bind to TIGIT and prevent activation by PVR (e.g., most commonly by blocking the interaction of TIGIT and PVR), are used to enhance T cell and/or NK cell activation and be used in treating diseases such as cancer and pathogen infection.

### V. ANTIBODIES

As is discussed below, the term “antibody” is used generally. Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention is directed to monoclonal antibodies that generally are based on the IgG class, which has several subclasses, including, but not limited to IgG1, IgG2, IgG3, and IgG4. In general, IgG1, IgG2 and IgG4 are used more frequently than IgG3. It should be noted that IgG1 has different allotypes with polymorphisms at 356 (D or E) and 358 (L or M). The sequences depicted herein use the 356D/358M allotype, however the other allotype is included herein. That is, any sequence inclusive of an IgG1 Fc domain included herein can have 356E/358L replacing the 356D/358M allotype. The term antibody further includes bispecific antibodies, for example, those antibodies that bind to at least two different targets. In some embodiments, the antibodies of the invention are bispecific antibodies that bind PVRIG and TIGIT (refereed to herein as anti-TIGIT/anti-PVRIG bispecific antibodies).

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3). A useful comparison of CDR numbering is as below, see Lafranc et al., Dev. Comp. Immunol. 27(1):55-77 (2003):

| | Kabat + | | | | | |
|---|---|---|---|---|---|---|
| | Clothia | IMGT | Kabat | AbM | Chothia | Contact |
| vhCDR1 | 26-35 | 27-38 | 31-35 | 26-35 | 26-32 | 30-35 |
| vhCDR2 | 50-65 | 56-65 | 50-65 | 50-58 | 53-55 | 47-58 |
| vhCDR3 | 95-102 | 105-117 | 95-102 | 95-102 | 96-101 | 93-101 |
| vlCDR1 | 24-34 | 27-38 | 24-34 | 24-34 | 26-32 | 30-36 |
| vlCDR2 | 50-56 | 56-65 | 50-56 | 50-56 | 50-52 | 46-55 |
| vlCDR3 | 89-97 | 105-117 | 89-97 | 89-97 | 91-96 | 89-96 |

Throughout the present specification, the Kabat numbering system is generally used when referring to a residue in the variable domain (approximately, residues 1-107 of the light chain variable region and residues 1-113 of the heavy chain variable region) and the hinge and the EU numbering system for Fc regions (e.g, Kabat et al., supra (1991)).

The present invention provides a large number of different CDR sets. In this case, a “full CDR set” comprises the three variable light and three variable heavy CDRs, e.g. a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used, or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and non-conformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Another type of Ig domain of the heavy chain is the hinge region. By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 220, and the IgG CH2 domain begins at residue EU position 237. Thus for IgG the antibody hinge is herein defined to include positions 221 (D221 in IgG1) to 236 (G236 in IgG1), wherein the numbering is according to the EU index as in Kabat.

The light chain generally comprises two domains, the variable light domain (containing the light chain CDRs and together with the variable heavy domains forming the Fv region), and a constant light chain region (often referred to as CL or Cκ). In general, either the constant lambda or constant kappa domain can be used, with lambda generally finding use in the invention.

Another region of interest for additional substitutions, outlined below, is the Fc region.

A. Chimeric and Humanized Antibodies

In some embodiments, the anti-PVRIG/anti-TIGIT bispecific antibodies herein can be derived from a mixture from different species, e.g. a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. Nos. 5,530,101; 5,585,089; 5,693,761; 5,693,762; 6,180,370; 5,859,205; 5,821,337; 6,054,297; 6,407,213, all entirely incorporated by reference). The humanized antibody optimally also will comprise at least a portion, and usually all, of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al.,1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci USA 89:4285-9, Presta et al., 1997, Cancer Res. 57(20):4593-9; Gorman et al., 1991, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference.

Thus, the vhCDRs and vlCDRs from any of the enumerated antibodies herein may be humanized (or “rehumanized”, for those that were already humanized).

In certain embodiments, the antibodies of the invention comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene excluding the CDRs. That is, the CDRs may be murine, but the framework regions of the variable region (either heavy or light) can be at least 96%, 97%, 98%, or 99% identical in amino acid sequence to the framework amino acids encoded by one human germline immunoglobulin gene.

Typically, a humanized antibody derived from a particular human germline sequence will display no more than 10-20 amino acid differences from the amino acid sequence encoded by the human germline immunoglobulin gene. In certain cases, the humanized antibody may display no more than 5, or even no more than 4, 3, 2, or 1 amino acid difference from the amino acid sequence encoded by the germline immunoglobulin gene (again, prior to the introduction of any variants herein; that is, the number of variants is generally low).

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

B. Optional Antibody Engineering

The anti-PVRIG/anti-TIGIT bispecific antibodies of the invention can be modified, or engineered, to alter the amino acid sequences by amino acid substitutions. As discussed herein, amino acid substitutions can be made to alter the affinity of the CDRs for the protein (e.g., TIGIT or PVRIG, including both increasing and decreasing binding), as well as to alter additional functional properties of the antibodies. For example, the antibodies may be engineered to include modifications within the Fc region, typically to alter one or more functional properties of the antibody, such as serum half-life, complement fixation, Fc receptor binding, and/or antigen-dependent cellular cytotoxicity. Furthermore, an antibody according to at least some embodiments of the invention may be chemically modified (e.g., one or more chemical moieties can be attached to the antibody) or be modified to alter its glycosylation, again to alter one or more functional properties of the antibody. Such embodiments are described further below. The numbering of residues in the Fc region is that of the EU index of Kabat.

In one embodiment, the hinge region of Cut is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In still another embodiment, the anti-PVRIG/anti-TIGIT bispecific antibodies can be modified to abrogate in vivo Fab arm exchange, in particular when IgG4 constant domains are used. Specifically, this process involves the exchange of IgG4 half-molecules (one heavy chain plus one light chain) between other IgG4 antibodies that effectively results in bispecific antibodies which are functionally monovalent. Mutations to the hinge region and constant domains of the heavy chain can abrogate this exchange (see Aalberse, R C, Schuurman J., 2002, Immunology 105:9-19). As outlined herein, a mutation that finds particular use in the present invention is the S241P in the context of an IgG4 constant domain. IgG4 finds use in the present invention as it has no significant effector function, and is thus used to block the receptor binding to its ligand without cell depletion (e.g. PVRIG to PVRL2 or TIGIT to PVR).

In some embodiments, amino acid substitutions can be made in the Fc region, in general for altering binding to FcγR receptors. By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII-1 (CD16), and FcγRIII-2 (CD16-2), as well as any undiscovered mouse FcγRs or FcγR isoforms or allotypes.

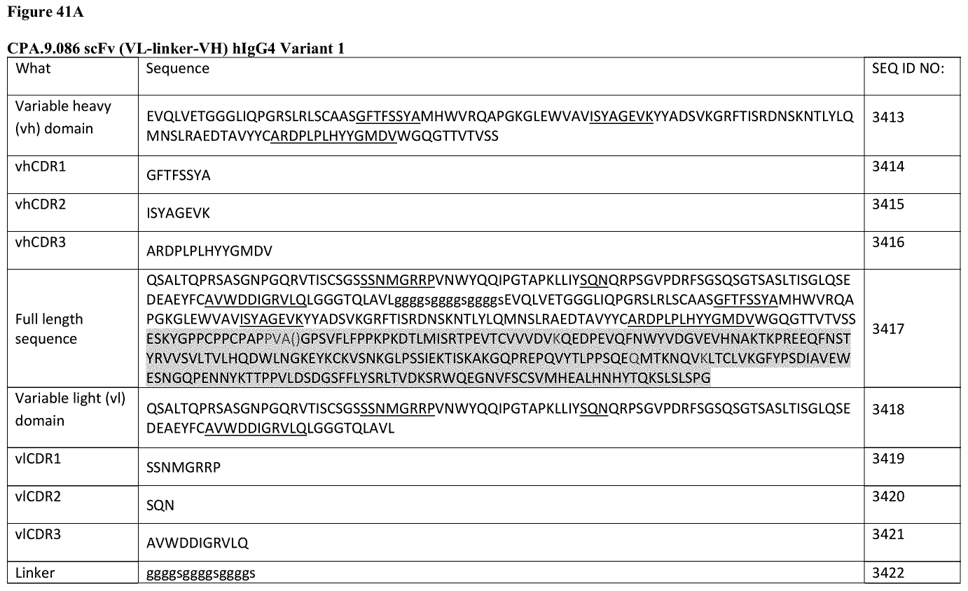

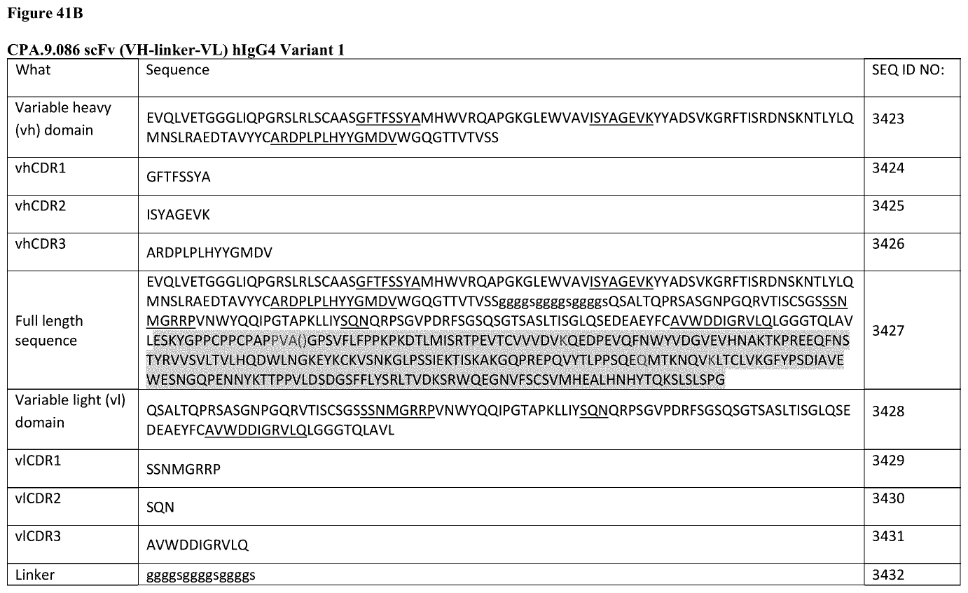

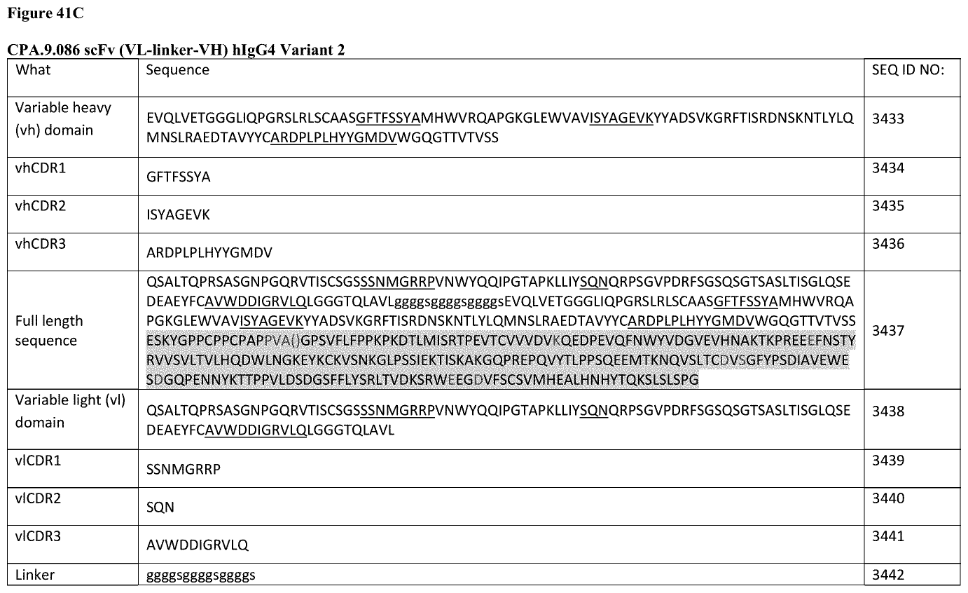

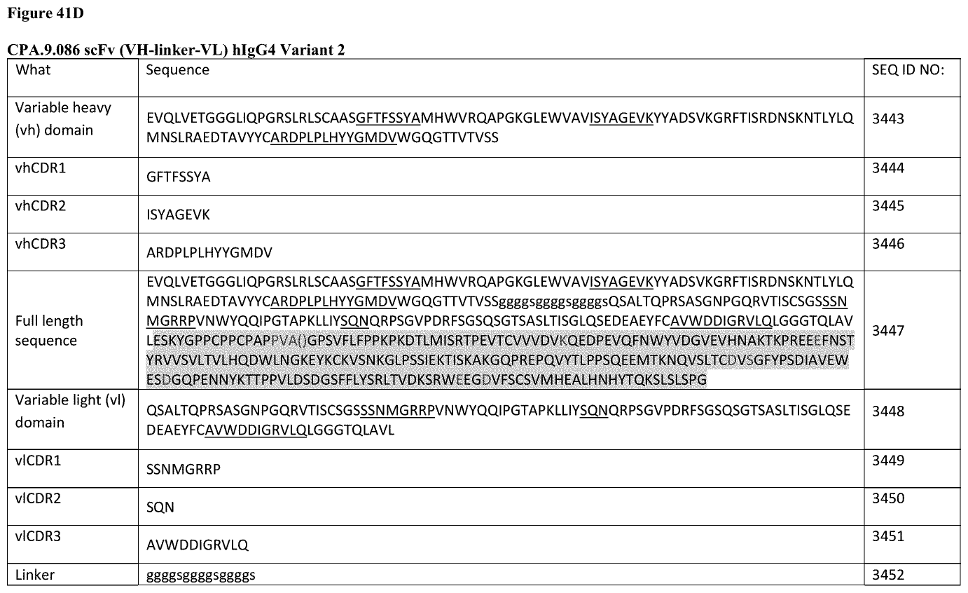

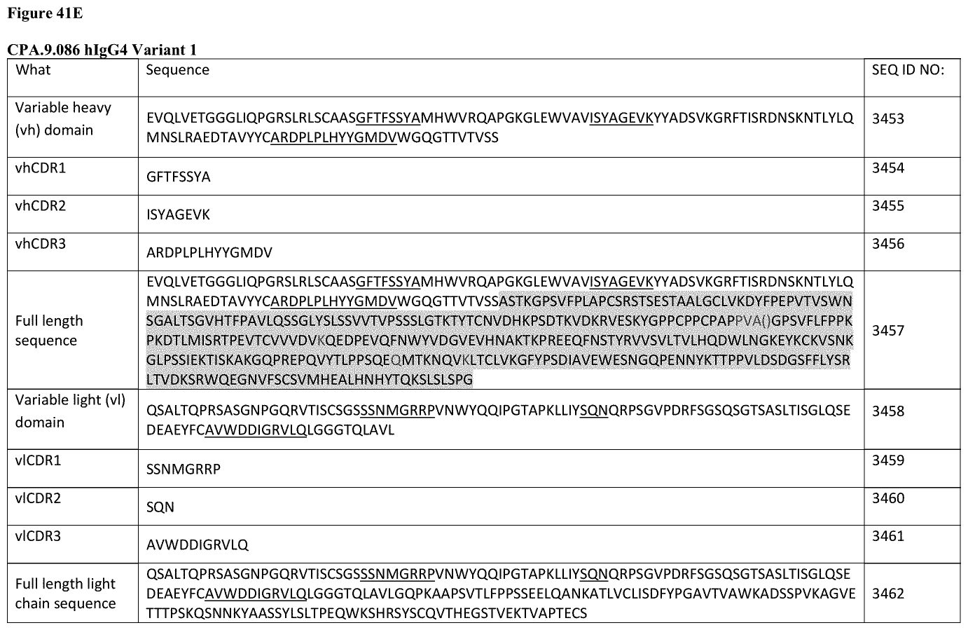

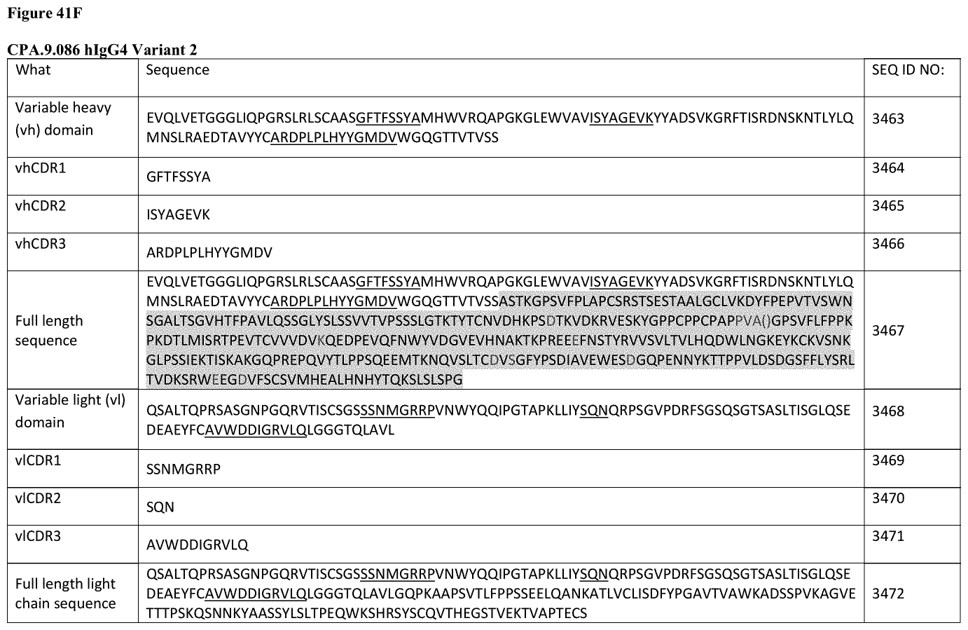

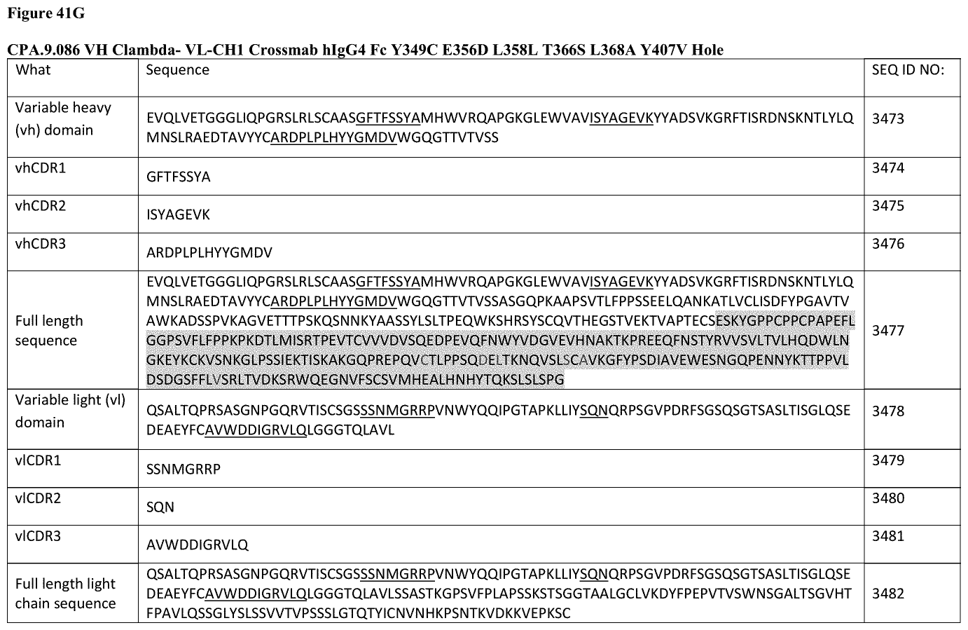

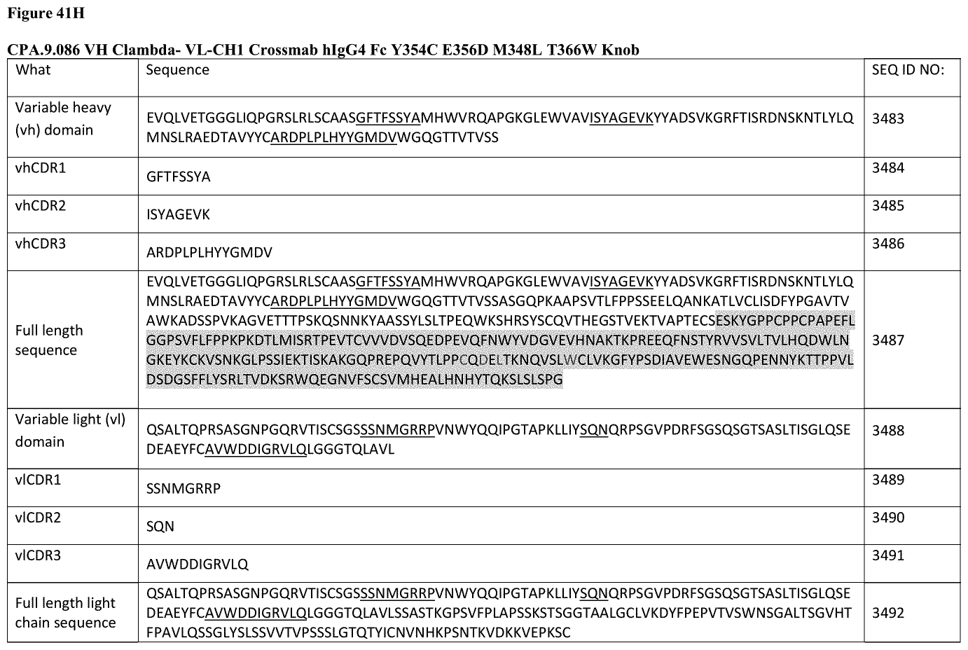

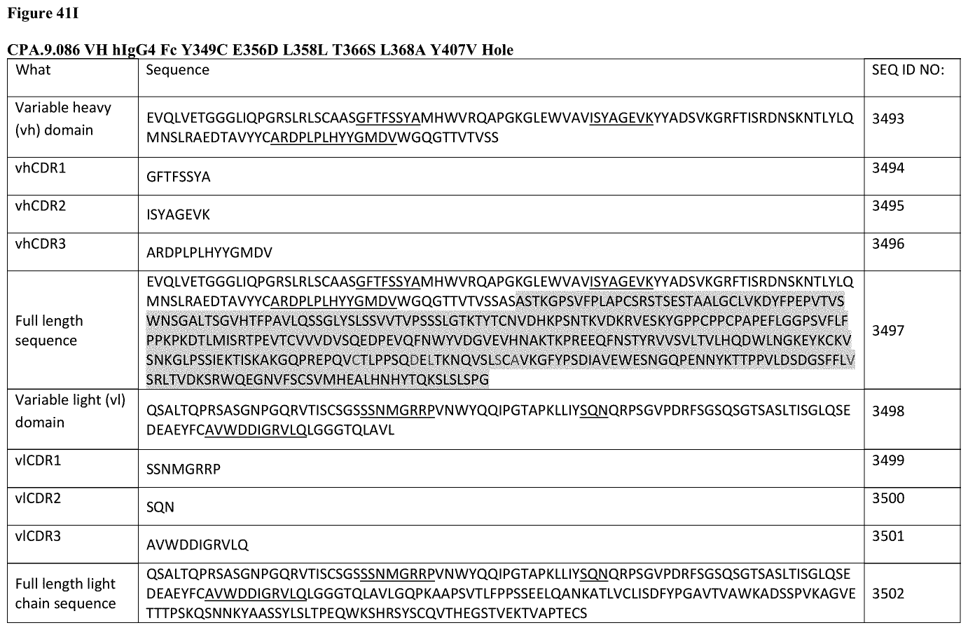

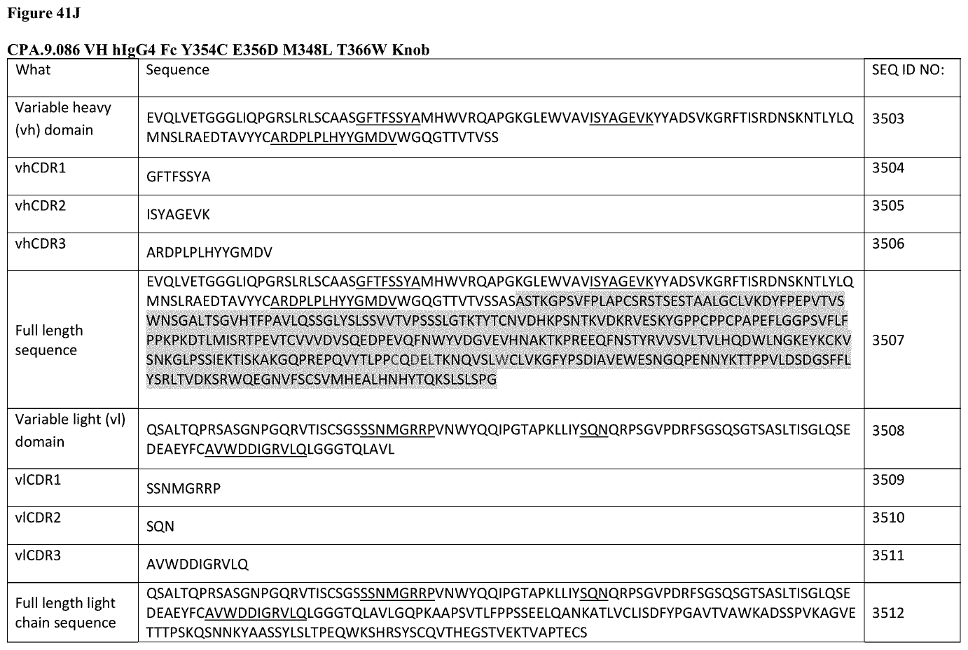

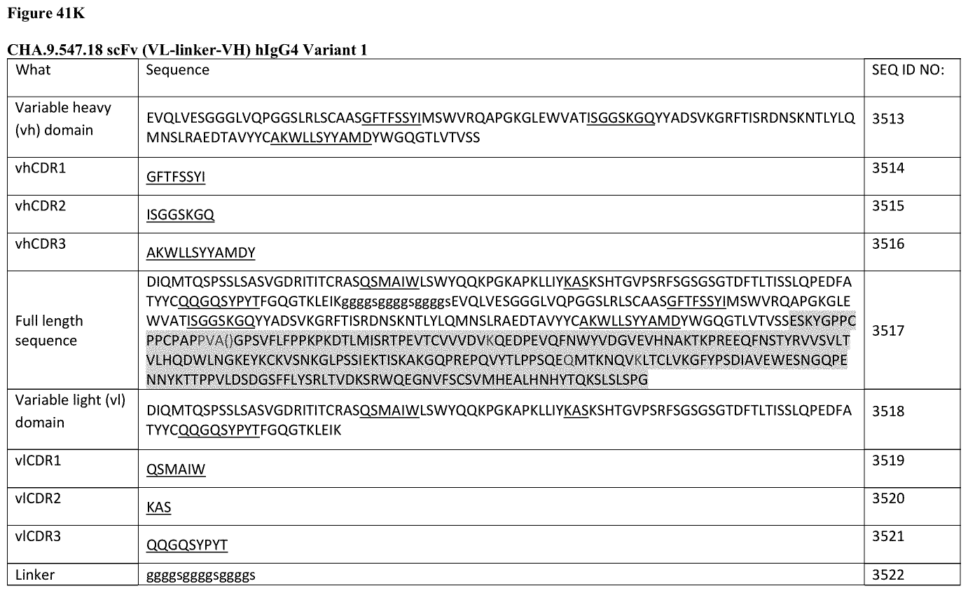

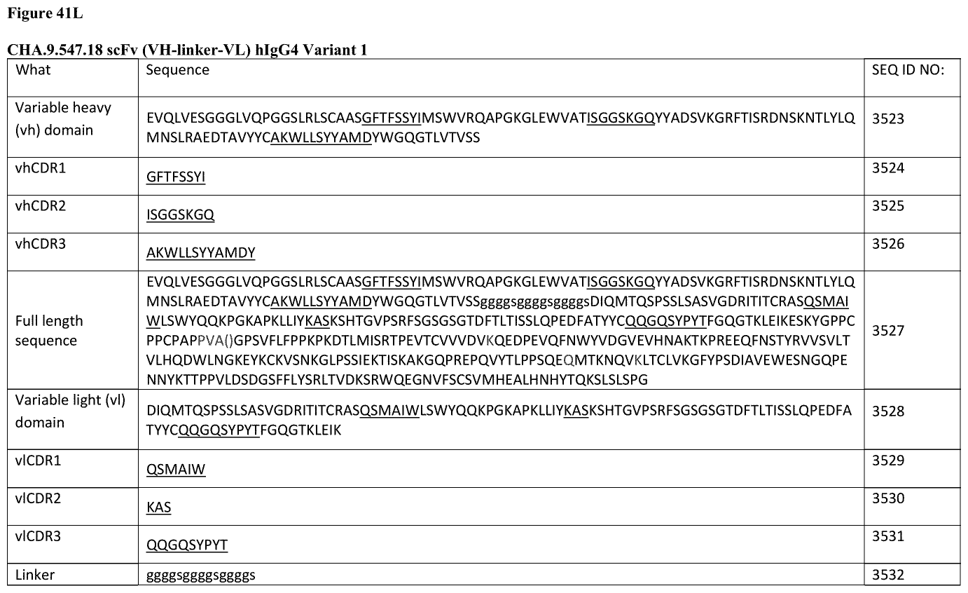

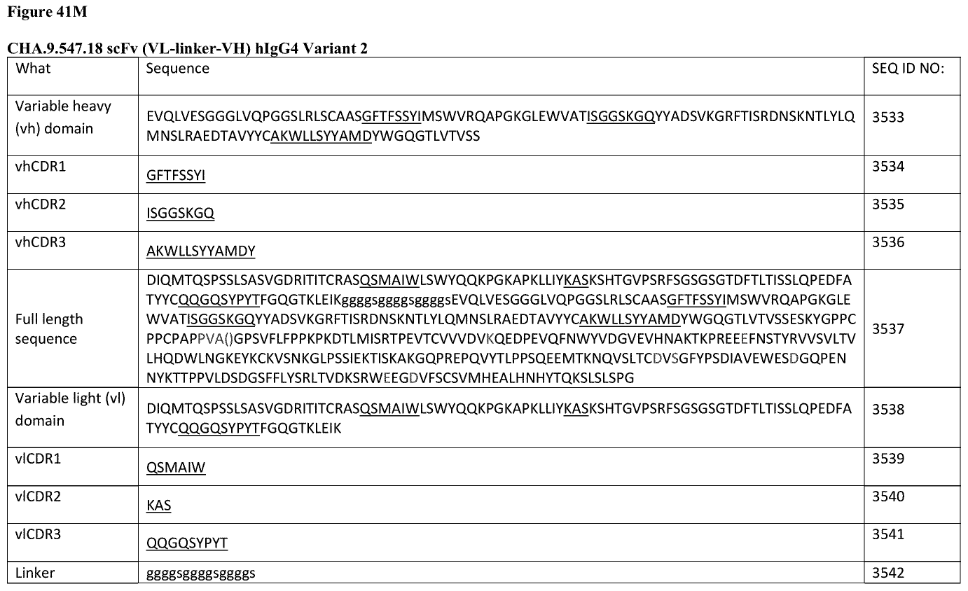

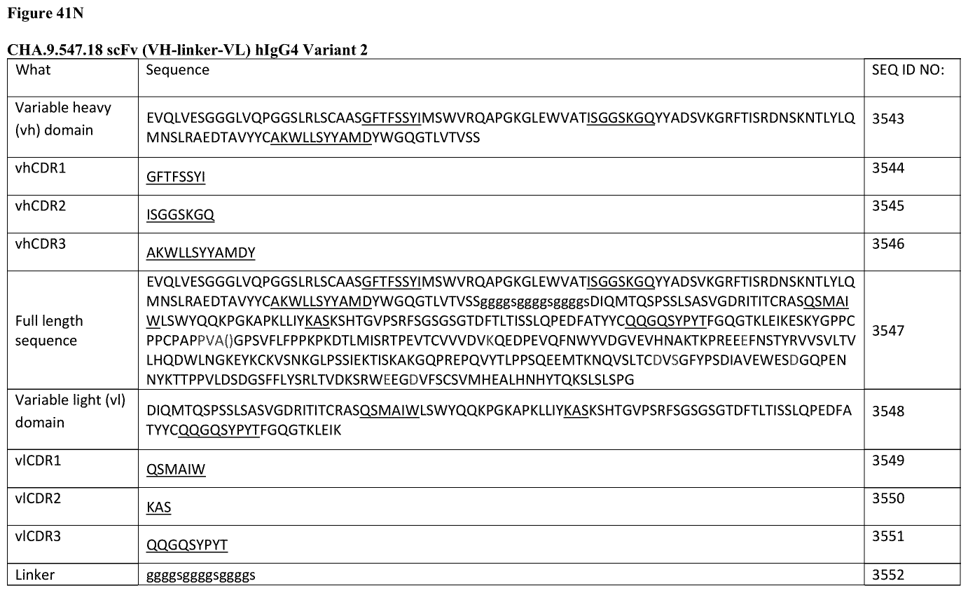

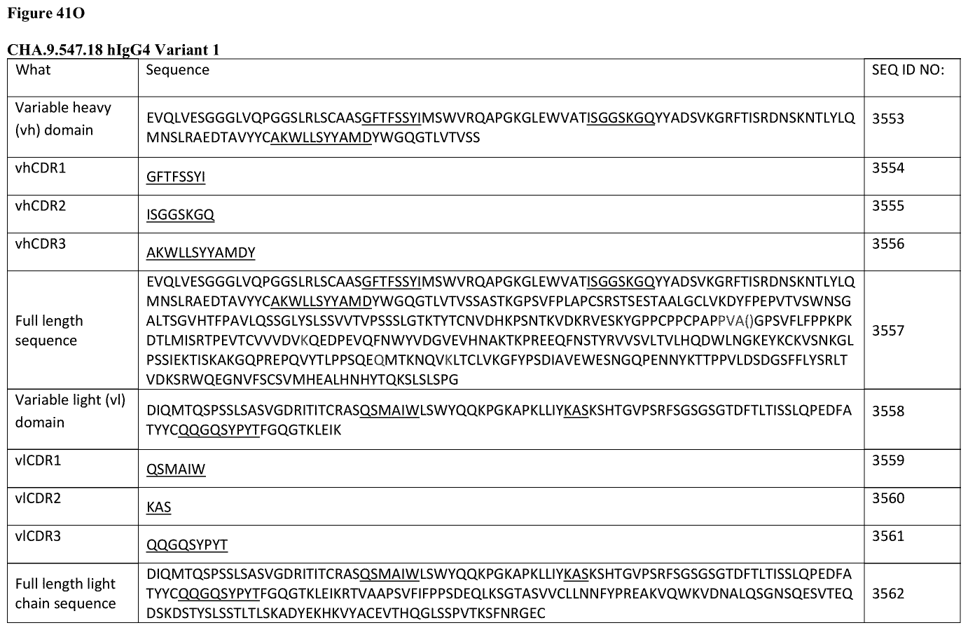

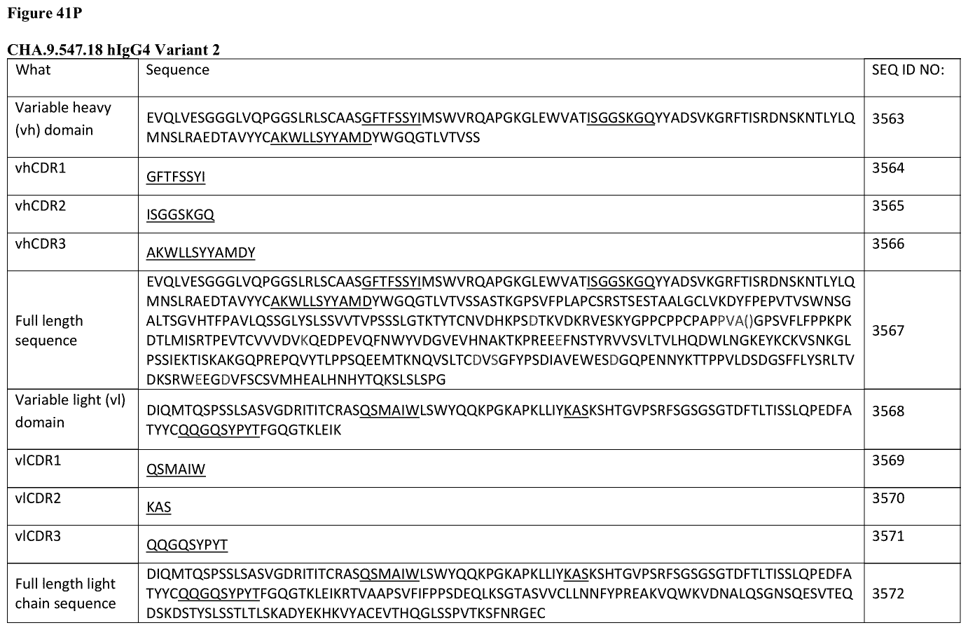

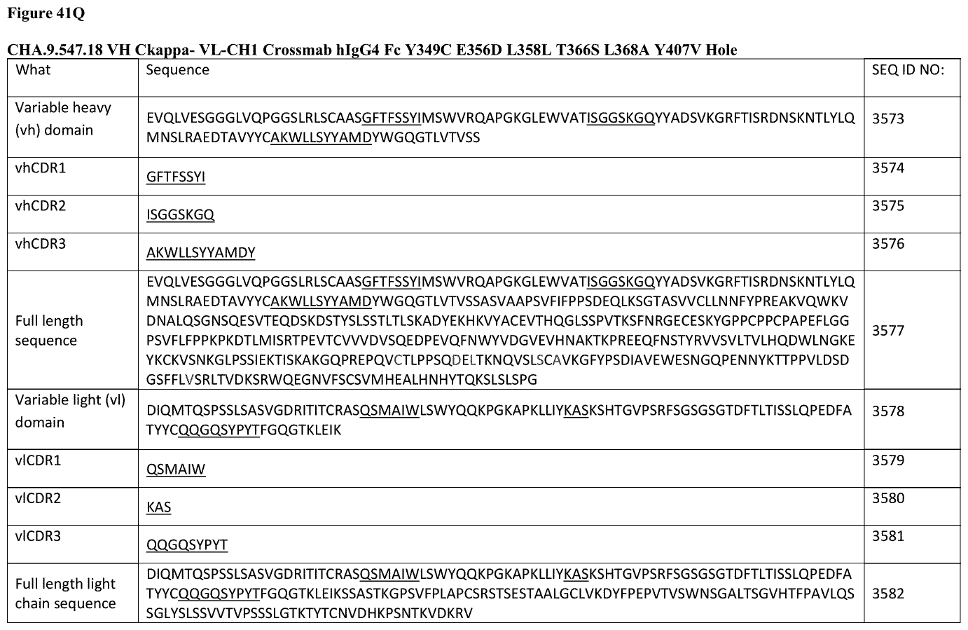

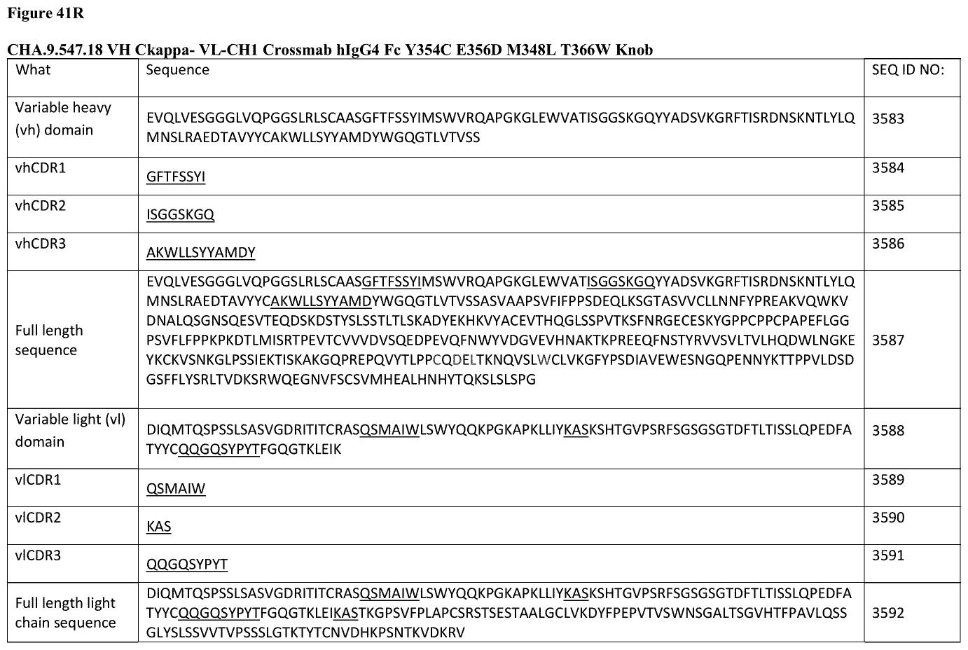

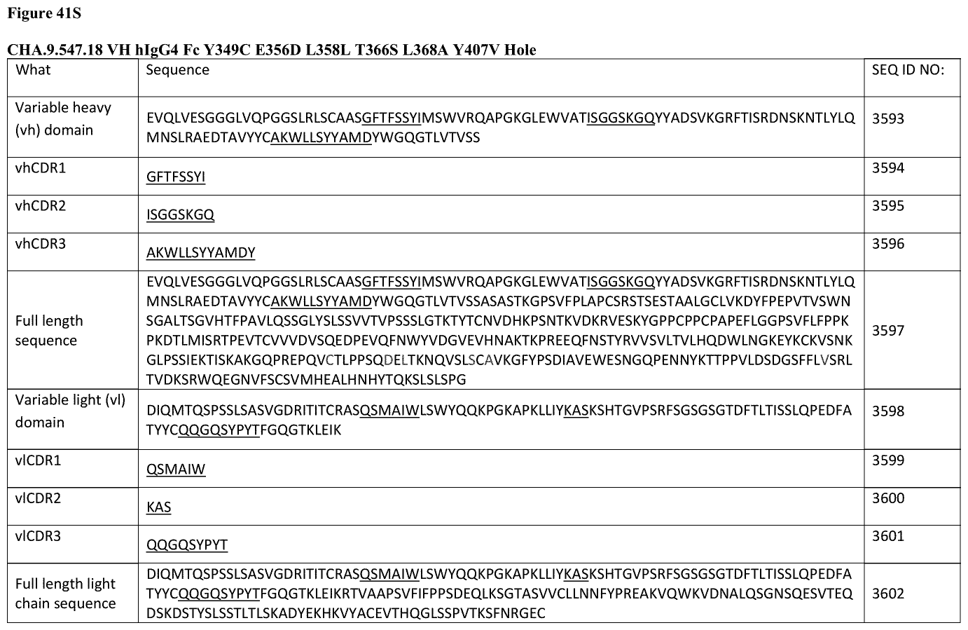

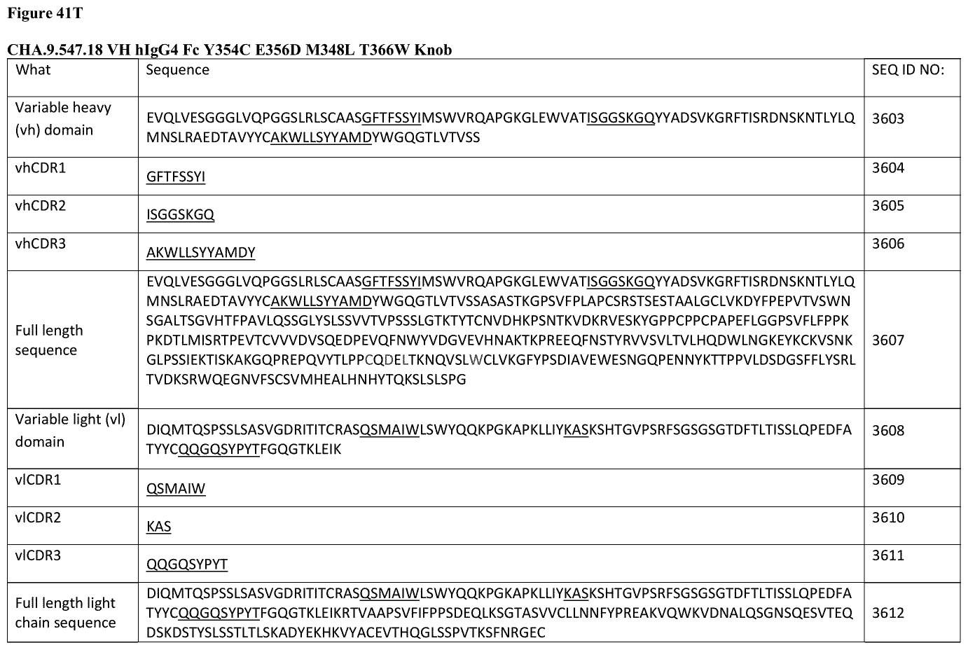

There are a number of useful Fc substitutions that can be made to alter binding to one or more of the FcγR receptors. Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell. Similarly, decreased binding to FcγRIIb (an inhibitory receptor) can be beneficial as well in some circumstances. Amino acid substitutions that find use in the present invention include those listed in U.S. Ser. No. 11/124,620 (particularly FIG. 41) and U.S. Pat. No. 6,737,056, both of which are expressly incorporated herein by reference in their entirety and specifically for the variants disclosed therein.

In yet another example, the Fc region is modified to increase the ability of the anti-PVRIG/anti-TIGIT bispecific antibodies to mediate antibody dependent cellular cytotoxicity (ADCC) and/or to increase the affinity of the antibody for an Fcγ receptor, and/or increase FcRn binding, by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 are shown to improve binding to FcγRIII. Additionally, the following combination mutants are shown to improve FcγRIII binding: T256A/S298A, S298A/E333A, S298A/K224A and S298A/E333A/K334A. Furthermore, mutations such as M252Y/S254T/T256E or M428L/N434S improve binding to FcRn and increase antibody circulation half-life (see Chan C A and Carter P J (2010) Nature Rev Immunol 10:301-316).

In addition, the anti-PVRIG/anti-TIGIT bispecific antibodies of the invention are modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the C_{H1} or C_{L} region to contain a salvage receptor binding epitope taken from two loops of a CH2 domain of an Fc region of an IgG, as described in U.S. Pat. Nos. 5,869,046 and 6,121,022 by Presta et al. Additional mutations to increase serum half-life are disclosed in U.S. Pat. Nos. 8,883,973, 6,737,056 and 7,371,826 and include 428L, 434A, 434S, and 428L/434S.

In still another embodiment, the glycosylation of an anti-PVRIG/anti-TIGIT bispecific antibody can be modified. For example, an aglycosylated antibody can be made (e.g., the antibody lacks glycosylation). Glycosylation can be altered to, for example, increase the affinity of the antibody for antigen or reduce effector function such as ADCC. Such carbohydrate modifications can be accomplished by, for example, altering one or more sites of glycosylation within the antibody sequence, for example N297. For example, one or more amino acid substitutions can be made that result in elimination of one or more variable region framework glycosylation sites to thereby eliminate glycosylation at that site, with an alanine replacement finding use in some embodiments.

Additionally or alternatively, an anti-PVRIG/anti-TIGIT bispecific antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies according to at least some embodiments of the invention to thereby produce an antibody with altered glycosylation. See for example, U.S. Patent Publication No. 20040110704 and WO 2003/035835.

Another modification of the anti-PVRIG/anti-TIGIT bispecific antibodies herein that is contemplated by the invention is PEGylation or the addition of other water soluble moieties, typically polymers, e.g., in order to enhance half-life. An antibody can be PEGylated to, for example, increase the biological (e.g., serum) half-life of the antibody as is known in the art.

In addition to substitutions made to alter binding affinity to FcγRs and/or FcRn and/or increase in vivo serum half-life, additional antibody modifications can be made, as described in further detail below.

In some cases, affinity maturation is done. Amino acid modifications in the CDRs are sometimes referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, it may be desirable to decrease the affinity of an antibody to its antigen.

In some embodiments, one or more amino acid modifications are made in one or more of the CDRs of the anti-PVRIG/anti-TIGIT bispecific antibodies of the invention (for example, to the PVRIG CDRs or the TIGIT CDRs). In general, only 1 or 2 or 3-amino acids are substituted in any single CDR, and generally no more than from 1, 2, 3. 4, 5, 6, 7, 8, 9, or 10 changes are made within a set of 6 CDRs (e.g., vhCDR1-3 and vlCDR1-3). However, it should be appreciated that any combination of no substitutions, 1, 2 or 3 substitutions in any CDR can be independently and optionally combined with any other substitution.

Affinity maturation can be done to increase the binding affinity of the antibody for the antigen by at least about 10% to 50-100-150% or more, or from 1 to 5 fold as compared to the “parent” antibody. In some embodiments, affinity matured antibodies will have nanomolar or even picomolar affinities for the antigen. Affinity matured antibodies are produced by known procedures. The correlation of affinity and efficacy is discussed below.

Alternatively, amino acid modifications can be made in one or more of the CDRs of the antibodies of the invention that are “silent”, e.g., that do not significantly alter the affinity of the antibody for the antigen. These can be made for a number of reasons, including optimizing expression (as can be done for the nucleic acids encoding the antibodies of the invention).

Thus, included within the definition of the CDRs and anti-PVRIG/anti-TIGIT bispecific antibodies of the invention are variant CDRs and anti-PVRIG/anti-TIGIT bispecific antibodies; that is, the anti-PVRIG/anti-TIGIT bispecific antibodies of the invention can include amino acid modifications in one or more of the CDRs of the enumerated antibodies of the invention. In addition, as outlined below, amino acid modifications can also independently and optionally be made in any region outside the CDRs, including framework and constant regions.

### VI. ANTI-PVRIG, ANTI-TIGIT, AND/OR ANTI-PVRIG/ANTI-TIGIT BISPECIFIC ANTIBODIES OF THE INVENTION

The present invention provides bispecific anti-PVRIG/anti-TIGIT antibodies, as well as anti-PVRIG and/or anti-TIGIT antibodies. (For convenience, “anti-PVRIG/anti-TIGIT antibodies” and “bispecific PVRIG/TIGIT antibodies” and “anti-PVRIG/anti-TIGIT bispecific antibodies” are used interchangeably). The anti-PVRIG/anti-TIGIT bispecific antibodies of the invention specifically bind to human TIGIT, and preferably the ECD of human TIGIT, as well as PVRIG, and again, preferably the ECD of human PVRIG. The invention further provides antigen binding domains, including full length antibodies, which contain a number of specific, enumerated sets of 12 CDRs, 6 CDRs that bind to TIGIT and 6 CDRs that bind to PVRIG.

The present invention also provides anti-PVRIG antibodies that can be in the context of a monospecific antibody or a bispecific antibody. Such antibodies include:

Antibody sequence for PVRIG
(IMGT CDR definition bold and underlined)
CHA.7.518.4 VH

(SEQ ID NO: 3179)

QVQLVQSGAEVKKPGASVKVSCKASGYTFTDYNINWVRQAPGQGLEW
MGYIYPYIGGSGYAQKFQGRVTMTRDTSTSTVYMELSSLRSEDTAVYY
CAREDKTARNAMDYWGQGTLVTVSS
CHA.7.518.4 VL

(SEQ ID NO: 3180)

DIQMTQSPSSLSASVGDRVTITCRVSENIYDVLAWYQQKPGKAPKLLIY
EATNLAEGVPSRFSGSGSGTDFTLTISSLQPEDFATYYCQHFWGTPYTF
GQGTKLEIK

The present invention also provides anti-TIGIT antibodies that can be in the context of a monospecific antibody or a bispecific antibody. Such antibodies include:

Antibody sequence for TIGIT
(IMGT CDR definition bold and underlined)
CHA.9.547.18 VH

(SEQ ID NO: 1664)

EVQLVESGGGLVQPGGSLRLSCAASGFTFSSYIMSWVRQAPGKGLEWV
ATISGGSKGQYYADSVKGRFTISRDNSKNTLYLQMNSLRAEDTAVYYC
AKWLLSYYAMDYWGQGTLVTVSS
CHA.9.547.18 VL

(SEQ ID NO: 1668)

DIQMTQSPSSLSASVGDRITITCRASQSMAIWLSWYQQKPGKAPKLLIY
KASKSHTGVPSRFSGSGSGTDFTLTISSLQPEDFATYYCQQGQSYPYT
FGQGTKLEIK

Specific binding for PVRIG and/or TIGIT or a PVRIG and/or TIGIT epitope can be exhibited, for example, by an antibody having a K_{D} of at least about 10⁻⁴ M, at least about 10⁻⁵ M, at least about 10⁻⁶ M, at least about 10⁻⁷ M, at least about 10⁻⁸ M, at least about 10⁻⁹M, alternatively at least about 10⁻¹⁰ M, at least about 10⁻¹¹ M, at least about 10⁻¹²M, at least about 10⁻¹³ M, at least about 10⁻¹⁴ M, at least about 10⁻¹⁵ M, or greater, where K_{D} refers to the equilibrium dissociation constant of a particular antibody-antigen interaction for each antigen independently. Typically, an antibody that specifically binds an antigen will have a K_{D} that is 20-, 50-, 100-, 500-, 1000-, 5,000-, 10,000- or more times greater for a control molecule relative to the TIGIT antigen or epitope.

However, for optimal binding to PVRIG and/or TIGIT expressed on the surface of NK and T-cells, the antibodies preferably have a KD less 50 nM and most preferably less than 1 nM, with less than 0.1 nM and less than 1 pM finding use in the methods of the invention

Also, specific binding for a particular antigen or an epitope can be exhibited, for example, by an antibody having a ka (referring to the association rate constant) for a PVRIG and/or a TIGIT antigen or epitope of at least 20-, 50-, 100-, 500-, 1000-, 5,000-, 10,000- or more times greater for the epitope relative to a control, where ka refers to the association rate constant of a particular antibody-antigen interaction.

In some embodiments, the anti-PVRIG/anti-TIGIT bispecific antibodies of the invention bind to human TIGIT and/or human PVRIG with a K_{D} of 100 nM or less, 50 nM or less, 10 nM or less, or 1 nM or less (that is, higher binding affinity), or 1 pM or less, wherein K_{D} is determined by known methods, e.g., surface plasmon resonance (SPR, e.g. Biacore assays), ELISA, KINEXA, and most typically SPR at 25° or 37° C.

A. Bispecific and/or Heterodimeric Antibodies

The present invention provides bispecific PVRIG and TIGIT checkpoint antibodies that rely on the use of two different heavy chain variant Fc sequences, which self-assemble to form Fc domains that are heterodimeric and antibodies that are heterodimeric antibodies (e.g., bispecific antibodies).

In some embodiments, the present invention provides anti-PVRIG/anti-TIGIT bispecific antibodies that allow for binding to both PVRIG and TIGIT. The antibody constructs described herein are based on the self-assembling and pairing of two Fc domains from two heavy chains (e.g., two Fc domains. two variable regions comprising the two Fc domains, and/or two heavy chains), to assembly into a dimer. In some embodiments, the amino acid sequences of each monomer are altered for to facilitate assembly of the monomers into dimers. In some embodiments, these amino acid variants and/or alterations are in the constant region. In some embodiments, the amino acid variants are different in each constant region in order to promote and/or facilitate heterodimeric assembly as compared to homodimeric assembly. Numerous methods and formats for bispecific antibodies are known in the art (see, for Example, Godar, et. al., Expert Opinion on Therapeutic Patents, 28(3):251-276 (2018) and Brinkmann and Kontermann, The making of bispecific antibodies, MAbs, 9(2): 182-212 (2017), both of which are incorporated by reference herein in their entireties).

As provided herein, the anti-PVRIG/anti-TIGIT bispecific heterodimeric antibodies of the invention include two antigen binding domains (ABDs), each of which bind to a different checkpoint protein, in particular PVRIG and TIGIT. These heterodimeric antibodies can be bispecific and bivalent (each antigen is bound by a single ABD, for example), or bispecific and trivalent (one antigen is bound by a single ABD and the other is bound by two ABDs). For all of the variable heavy and light domains listed herein, further variants can be made. In some embodiments, the anti-PVRIG/anti-TIGIT bispecific antibodies of the invention can comprise ABDs from any of the variable heavy and light domains and/or chains listed herein, such as for example in FIGS. 4A-4AA, 7A-7AE, 8A-8D, 11A-11I, 12A-12E, 15A15B, 16A-16E, 17A-17C, 23A-23D, 24A-24EE, 40A-40T, and 41A-41T. In some embodiments, the anti-PVRIG/anti-TIGIT bispecific antibodies of the invention can comprise CDRs from any of the variable heavy and light domains and/or chains listed herein, such as for example in FIGS. 4A-4AA, 7A-7AE, 8A-8D, 11A-11I, 12A-12E, 15A15B, 16A-16E, 17A-17C, 23A-23D, 24A-24EE, 40A-40T, and 41A-41T. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 80, 85 or 90% identity to a human germline sequence. Thus, for example, the identical CDRs as described herein can be combined with different framework sequences from human germline sequences, as long as the framework regions retain at least 80, 85 or 90% identity to a human germline sequence. Alternatively, the CDRs can have amino acid modifications (e.g. from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.), as well as having framework region changes, as long as the framework regions retain at least 80, 85 or 90% identity to a human germline sequence.

The present invention provides anti-PVRIG/anti-TIGIT bispecific antibodies. Bispecific antibodies are generally made by expressing genes for each heavy and light chain in the host cells. This generally results in the formation of the desired heterodimer (A-B), as well as the two homodimers (A-A and B-B (not including the light chain heterodimeric issues)).

In order to address the issues surrounding homodimeric and heterodimeric antibody formation (and the issues around separation of the two during purification), methods have been developed to bias self-assembly into the heterodimeric forms as opposed to the homodimeric forms. A number of mechanism and methods can be used to generate heterodimeric antibodies and in particular to generate heterodimers using the PVRIG and TIGIT antibody sequences of the present invention, including those FIGS. 4A-4AA, 7A-7AE, 8A-8D, 11A-11I, 12A-12E, 15A15B, 16A-16E, 17A-17C, 23A-23D, 24A-24EE, 40A-40T, and 41A-41T. In some embodiments, the anti-PVRIG/anti-TIGIT bispecific antibody comprise the sequences provided in FIGS. 26A-26B, 40A-40T, and 41A-41T (SEQ IN NOs:3213-3612). In some embodiments, mechanisms and methods are combined to ensure a high percentage of heterodimerization.

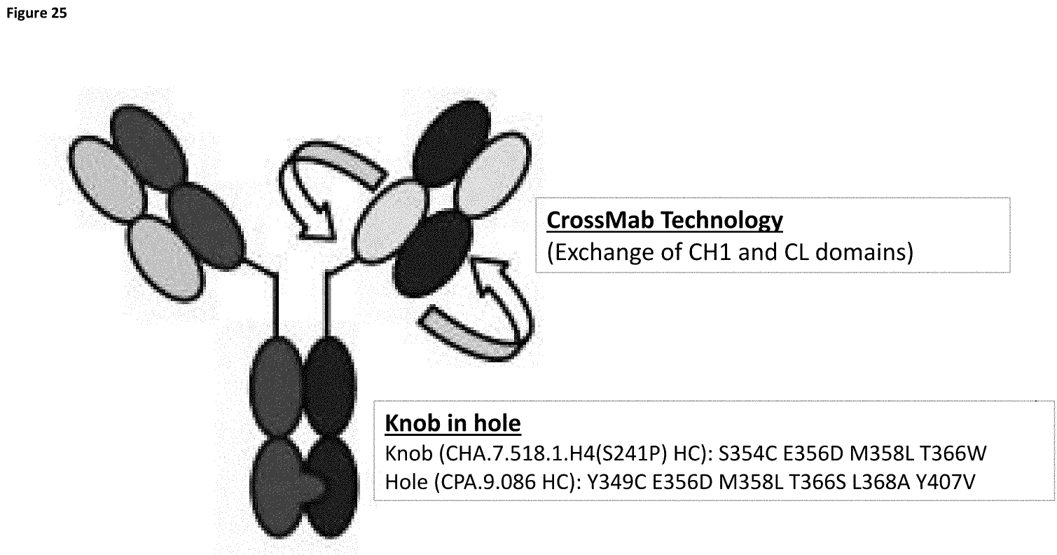

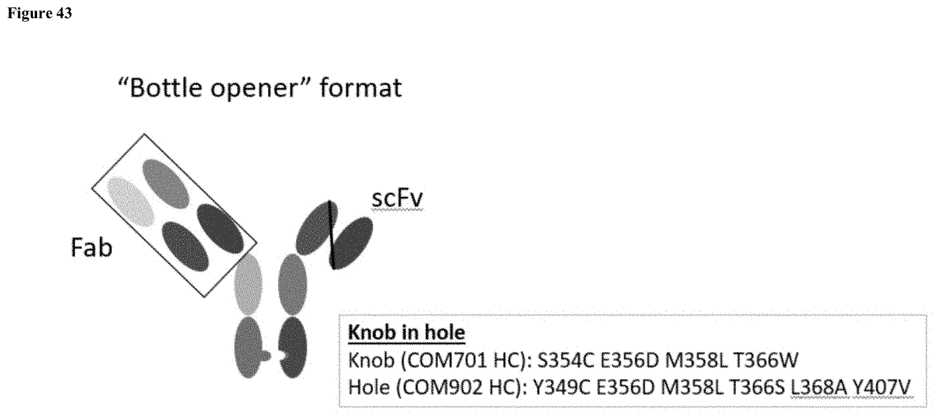

Variants that can promote heterodimerization variants (sometimes referred to as “heterodimerization variants) can include steric variants (e.g., the “knobs and holes” as provided as an example in FIGS. 25, 42, and 43, or “skew” variants described below and the “charge pairs” variants described below) as well as “pi variants”, which allow for purification of homodimers away from heterodimers. Such methods have been described in a variety of patent publications, including WO1996/027011 as well as WO2014/145806, hereby incorporated by reference in its entirety for the discussion of “heterodimerization variants”, useful mechanisms for heterodimerization including “knobs and holes” (“KIH”; sometimes herein as “skew” variants, “electrostatic steering” or “charge pairs” as described in WO2006/106905 or WO2014/145806, pi variants as described in WO 2007/114325 or WO2014/145806, as well as WO2017/218707, and WO2018045110; all of which are incorporated by reference herein in their entireties.

1. Heterodimerization Variants

The present invention provides anti-PVRIG/anti-TIGIT bispecific antibodies antibodies in a variety of formats, which utilize heterodimeric variants to allow for heterodimeric formation and/or purification away from homodimers.

There are a number of suitable pairs of sets of heterodimerization skew variants. These variants come in “pairs” of “sets”. In some embodiments, one set of the pair is incorporated into the first monomer and the other set of the pair is incorporated into the second monomer. In some embodiments, these sets do not necessarily behave as “knobs in holes” variants, with a one-to-one correspondence between a residue on one monomer and a residue on the other, but rather these pairs of sets form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). In some embodiments, the percentage of heterodimers formed is greater than 90%, greater than 91%, greater than 92%, greater than 93%, greater than 94%, greater than 95%, greater than 96%, greater than 97%, greater than 98%, or greater than 99%.

In some embodiments, the formation of heterodimers can be facilitated by the addition of steric variants. In some embodiments, by altering amino acids in each heavy chain, different heavy chains are more likely to associate to form the heterodimeric structure than to form homodimers with the same Fc amino acid sequences (e.g., to prevent HC/HC mispairing). Suitable steric variants are known in the art and discussed in further detail below.

In some embodiments, a mechanism referred to as “knobs and holes” or “knobs into holes”, referring to amino acid engineering that creates steric influences that promote heterodimeric formation and disfavor homodimeric formation can also optionally be used; this is sometimes referred to as “knobs and holes”, as described in Ridgway et al., Protein Engineering 9(7):617 (1996); Atwell et al, J. Mol. Biol. 1997 270:26; U.S. Pat. No. 8,216,805; and WO1996/027011, all of which are hereby incorporated by reference in their entirety. In some embodiments the “knobs” refers to a CH3 domain a variant, T366Y, and the “holes” refers to a CH3 domain b variant, Y407T. In some embodiments the “knobs” refers to a CH3 domain a variant, S354C/T366W, and the “holes” refers to a CH3 domain b variant, Y349C/T366S/L368A/Y407V. In addition, as described in Merchant et al, Nature Biotech. 16:677 (1998), these “knobs and holes” mutations can be combined with disulfide bonds to skew formation to heterodimerization. In some embodiments, the PVRIG binding portion comprises one set of substitutions from the pair and the TIGIT binding portion comprises the other set of substitutions from the pair. In some embodiments, the PVRIG binding portion comprises the “knobs” substitutions and the TIGIT binding portion comprises the “hole” substitutions from the pair. In some embodiments, the TIGIT binding portion comprises the “knobs” substitutions and the PVRIG binding portion comprises the “hole” substitutions from the pair. In some embodiments, the PVRIG binding portion comprises the substitutions S354C/T366W and the TIGIT binding portion comprises the substitutions Y349C/T366S/L368A/Y407V. In some embodiments, the PVRIG binding portion comprises the substitutions S354C/E356D/M358L/T366W and the TIGIT binding portion comprises the substitutions Y349C/E356D/M358L/T366S/L368A/Y407V.

In some embodiments, a mechanism referred to as “ART-Ig” (Asymmetric Re-engineering Technology-Immunoglobulin), referring to amino acid engineering that which introduces mutations to create electrostatic steering effects can also optionally be used; for example, these address LC/HC pairing (common LCs by framework/complementarity determining regions shuffling) and HC/HC mispairing problems solved by introducing mutations to create electrostatic steering effects. In some embodiments, the PVRIG binding portion comprises one set of substitutions from the pair and the TIGIT binding portion comprises the other set of substitutions from the pair. Such methods are described in Gunasekaran et al., J. Biol. Chem. 285(25): 19637 (2010); and WO2006/106905, hereby incorporated by reference in their entireties. This method is also sometimes referred to “charge pairs”. In some embodiments, electrostatics are used to skew the formation towards heterodimerization. These substitutions can also have an effect on pi, and thus on purification, and in some embodiments, could also be considered pi variants. In some embodiments, such substitutions are considered and/or referred to as “steric variants”. In some embodiments, these include IgG1 hinge/CH3 charge pairs (EEE-RRR) comprising the set of substitutions D221E/P228E/L368E, paired with D221R/P228R/K409R. In some embodiments, these include IgG2 hinge/CH3 charge pairs (EEE-RRRR) comprising the set of substitutions C223E/P228E/L368E paired with C223R/E225R/P228R/K409R. In some embodiments, these include CH3 charge pairs (DD-KK) K392D/K409D paired with E356K/D399K. In some embodiments, these include EW-RVT pairs, with K360E/K409W paired with Q347R/D399V/F405T. In some embodiments, these include EW-RVTS-S pairs, with K360E/K409W/Y349C paired with Q347R/D399V/F405T/S354C. In some embodiments, these include 366K (+351K) paired with 351D or E or D at 349, 368, 349, or 349+355. In some embodiments, these include DuoBody (L-R) pairs, with F405L paired with K409R. In some embodiments, these include SEEDbody pairs, with IgG/A chimera paired with an IgG/A chimera. In some embodiments, these include BEAT pairs, with residues from TCRα interface paired with residues from TCRβ interface. In some embodiments, these include BEAT pairs, with residues from TCRα interface in CH3 domain a paired with residues from TCRβ interface in CH3 domain b. In some embodiments, these include 7.8.60 (DMA-RRVV) pairs, with K360D/D399M/Y407A paired with E345R/Q347R/T366V/K409V. In some embodiments, these include 20.8.34 (SYMV-GDQA) pairs, with Y349S/K370Y/T366M/K409V paired with E356G/E357D/S364Q/Y407A. See, for example, Brinkmann and Kontermann, The making of bispecific antibodies, MAbs, 9(2): 182-212 (2017). In some embodiments, the PVRIG binding portion comprises one set of substitutions from the pair and the TIGIT binding portion comprises the other set of substitutions from the pair. See, for example, WO2012131555, and WO2011131746, incorporated by reference herein in their entireties.

In some embodiments, a mechanism referred to as CrossMAb can be employed to address LC/HC mispairing issues. In some embodiments, CrossMAb^{VH-VL} is employed to exchange the VH and VL domains. In some embodiments, CrossMAb^{CH1-CL} is employed to exchange the CH1 and CL domains. In some embodiments, CrossMAb^{Fab} is employed to exchange the VH-CH1 and VL-CL domains. In some embodiments, the anti-PVRIG/anti-TIGIT bispecific heterodimeric antibodies of the invention employs the CrossMAb^{CH1-CL} and the CH1 and CL domains are exchanged. In some embodiments, the anti-PVRIG/anti-TIGIT bispecific heterodimeric antibodies of the invention employs the CrossMAb^{VH-VL} and the VH and VL domains are exchanged. In some embodiments, the anti-PVRIG/anti-TIGIT bispecific heterodimeric antibodies of the invention employs the CrossMAb^{Fab} and the VH-CH1 and VL-CL domains are exchanged. See, for example, WO2009080251, incorporated by reference herein in its entirety.

In some embodiments, a mechanism referred to as BiMAb can be employed to address LC/HC and HC/HC mispairing issues and promote formation of the desired bispecific antibody. See, for example, WO2010129304, incorporated by reference herein in its entirety. In some embodiments, LC/HC and HC/HC mispairing problems can be solved by introducing mutations to create electrostatic steering effects, e.g., in a human IgG2. In some embodiments, these include BiMAb pairs, with CH3 domain a substitutions K249E/K288E paired with CH3 domain b substitutions E236K/D278K. In some embodiments, the PVRIG binding portion comprises one set of substitutions from the BiMAb pair and the TIGIT binding portion comprises the other set of substitutions from the BiMAb pair.

In some embodiments, a mechanism referred to as FcΔAdp can be employed to address LC/HC and HC/HC mispairing issues and promote formation of the desired bispecific antibody. See, for example, WO2010151792, incorporated by reference herein in its entirety. In some embodiments, LC/HC and HC/HC mispairing problems can be solved by introducing mutations to create differential protein A affinity. In some embodiments, these include FcΔAdp pairs, with CH3 domain a substitution H435R paired with no substitutions in the CH3 domain. In some embodiments, the PVRIG binding portion comprises one set of substitutions from the FcΔAdp pair and the TIGIT binding portion comprises the other set of substitutions from the FcΔAdp pair.

In some embodiments, a mechanism referred to as XmAb can be employed to address LC/HC (Fab-scFv-Fc) and HC/HC mispairing issues and promote formation of the desired bispecific antibody. See, for example, WO2011028952, incorporated by reference herein in its entirety. In some embodiments LC/HC (Fab-scFv-Fc) and HC/HC mispairing problems can be solved by introducing HA-TF substitutions. In some embodiments, the HA-TF pair includes CH3 domain a substitutions S364H/F405A with CH3 domain b substitutions Y349T/T394F. In some embodiments, the PVRIG binding portion comprises one set of substitutions from the XmAb pair and the TIGIT binding portion comprises the other set of substitutions from the XmAb pair.

In some embodiments, a mechanism referred to as DuoBody can be employed to address LC/HC (controlled Fab-arm exchanged) and HC/HC mispairing issues and promote formation of the desired bispecific antibody. See, for example, WO2011131746, incorporated by reference herein in its entirety. In some embodiments, LC/HC and HC/HC mispairing problems can be solved by introducing CH3 domain substitutions. In some embodiments, the DuoBody (L-R) pair includes a CH3 domain a substitution F405L paired with a CH3 domain b substitution K409R. In some embodiments, the PVRIG binding portion comprises one of the substitutions from the DuoBody pair and the TIGIT binding portion comprises the other substitution from the DuoBody pair.

In some embodiments, a mechanism referred to as Azymetric can be employed to address LC/HC (orthoFab-Ig) and HC/HC mispairing issues and promote formation of the desired bispecific antibody. See, for example, WO2012058768, incorporated by reference herein in its entirety. In some embodiments, mispairing problems can be solved by introducing ZW1 substitutions. In some embodiments, the ZW1 pair includes CH3 domain a substitutions T350V/L351Y/S400E/F405A/Y407V paired with CH3 domain b substitutions T350V/T366L/N390R/K392M/T394W. In some embodiments, the PVRIG binding portion comprises one of the substitutions from the ZW1 pair and the TIGIT binding portion comprises the other substitution from the ZW1 pair.

In some embodiments, a mechanism referred to as Biclonics can be employed to address LC/HC (common LCs generated by using the transgenic mouse MeMo and phage display libraries) and HC/HC mispairing mispairing issues and promote formation of the desired bispecific antibody. See, for example, WO2013157953, incorporated by reference herein in its entirety. In some embodiments, mispairing problems can be solved by introducing various substitutions. In some embodiments, the substitution pair includes CH3 domain a substitution T366K (+L351K) paired with CH3 domain b substitutions L351D/E or D at any one of Y349, L368, or Y349, and +R355. In some embodiments, the PVRIG binding portion comprises one of the substitutions from the Biclonics pair and the TIGIT binding portion comprises the other substitution from the Biclonics pair.

In some embodiments, the steric variants discussed herein can be optionally and independently incorporated with any pi or other variants such as Fc variants, FcRn variants, etc., into one or both monomers, and can be independently and optionally included or excluded from the anti-PVRIG/anti-TIGIT bispecific antibodies of the invention.

2. Exemplary Bispecific Antibody Formats

As would be understood by those skilled in the art and discussed more fully below, the anti-TIGIT/anti-PVRIF bispecific heterodimeric antibodies of the present invention can occur in a variety of configurations, as provided in FIGS. 33 (asymmetric formats) and 34 (symmetric formats) (see, also, Brinkmann and Kontermann, The making of bispecific antibodies, MAbs, 9(2): 182-212 (2017); incorporated by reference herein). In some embodiments, the heterodimeric formats of the anti-TIGIT/anti-PVRIF bispecific antibodies of the invention can have different valencies as well as be bispecific. In some embodiments, the anti-TIGIT/anti-PVRIF bispecific heterodimeric antibodies of the invention can be bivalent and bispecific, wherein one checkpoint target (e.g., either PVRIG or TIGIT) is bound by one binding portion or antigen binding domain (ABD) and the other checkpoint target (e.g., either TIGIT or PVRIG) is bound by a second binding portion or antigen binding domain (ABD). In some embodiments, the anti-TIGIT/anti-PVRIF bispecific heterodimeric antibodies of the invention can also be trivalent and bispecific, wherein the first checkpoint target (e.g., either PVRIG or TIGIT) is bound by two binding portions or antigen binding domains (ABDs) and the second checkpoint target (e.g., either PVRIG or TIGIT) by a second binding portion or antigen binding domain (ABD).

In some embodiments, the format is any one of those provided, for example in FIGS. 33 and 34, as well as WO2015/149077A1 and WO2017/218707, incorporated by reference herein in its entirety. These formats can be combined with any of the above variants as well.

B. PVRIG Binding Portion of the Anti-PVRIG/Anti-TIGIT Bispecific Antibodies

Specific binding for PVRIG or a PVRIG epitope can be exhibited, for example, by an antibody having a KD of at least about 10⁻⁴M, at least about 10⁻⁵ M, at least about 10⁻⁶ M, at least about 10⁻⁷ M, at least about 10⁻⁸M, at least about 10⁻⁹M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the PVRIG antigen or epitope.

Generally, for optimal binding to PVRIG expressed on the surface of NK and T-cells, the antibodies preferably have a KD less 50 nM and most preferably less than 1 nM, with less than 0.1 nM and less than 1 pM and 0.1 pM finding use in the methods of the invention.

Also, specific binding for a particular antigen or an epitope can be exhibited, for example, by an antibody having a KA or Ka for a PVRIG antigen or epitope of at least 20-, 50-, 100-, 500-, 1000-, 5,000-, 10,000- or more times greater for the epitope relative to a control, where KA or Ka refers to an association rate of a particular antibody-antigen interaction.

In some embodiments, the anti-PVRIG and/or anti-PVRIG/anti-TIGIT bispecific antibodies of the invention bind to human PVRIG with a K_{D} of 100 nM or less, 50 nM or less, 10 nM or less, or 1 nM or less (that is, higher binding affinity), or 1 pM or less, wherein K_{D} is determined by known methods, e.g. surface plasmon resonance (SPR, e.g. Biacore assays), ELISA, KINEXA, and most typically SPR at 25° or 37° C.

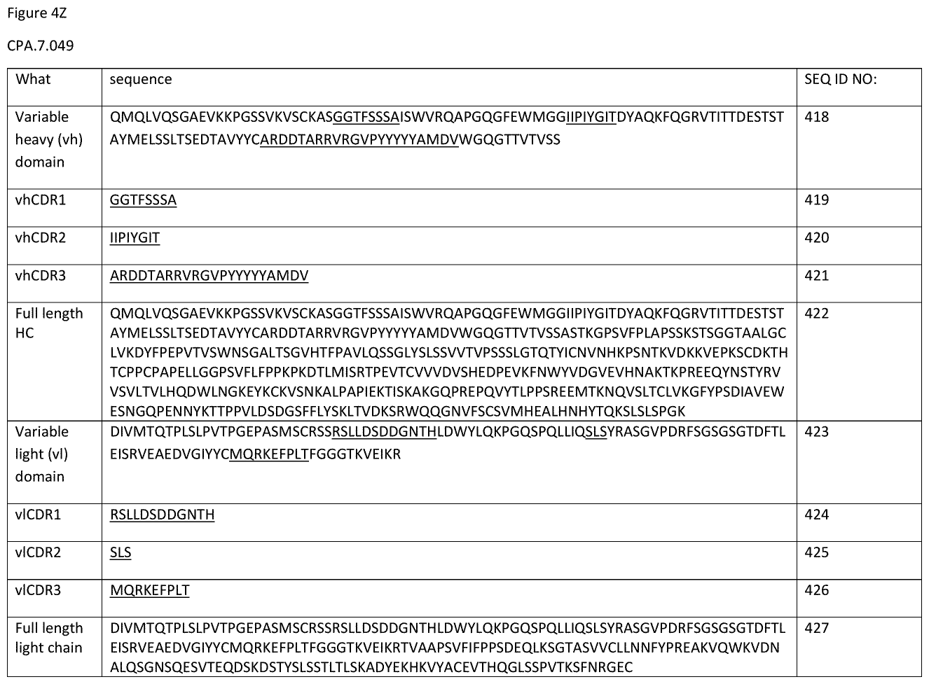

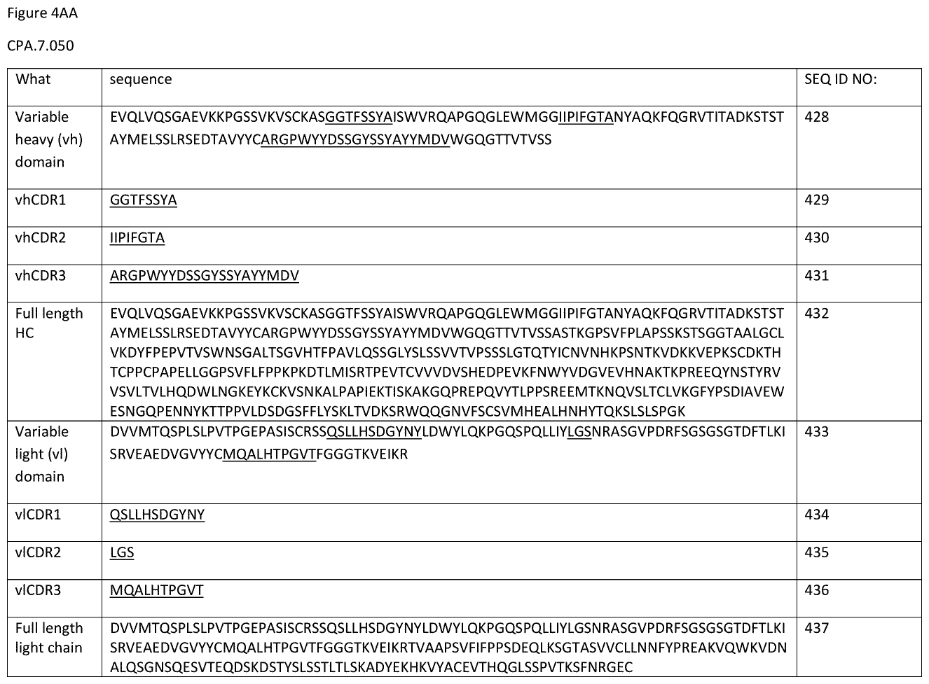

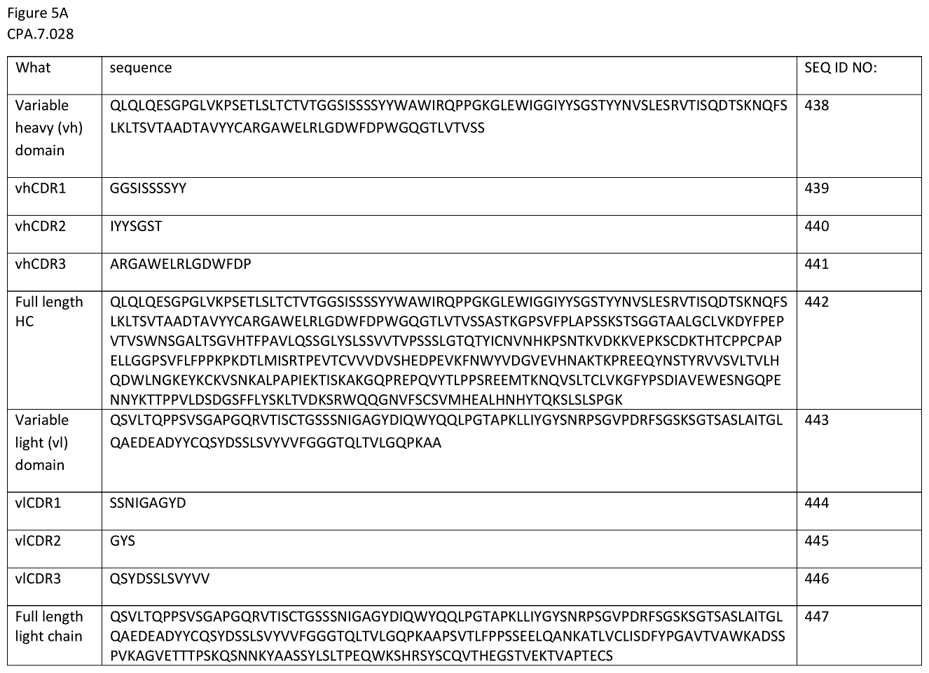

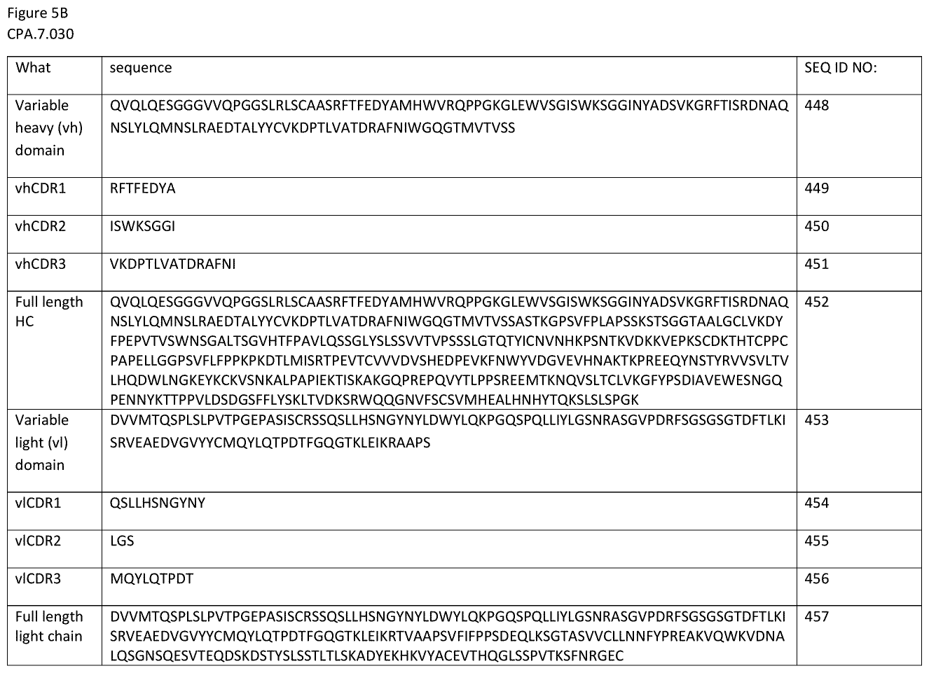

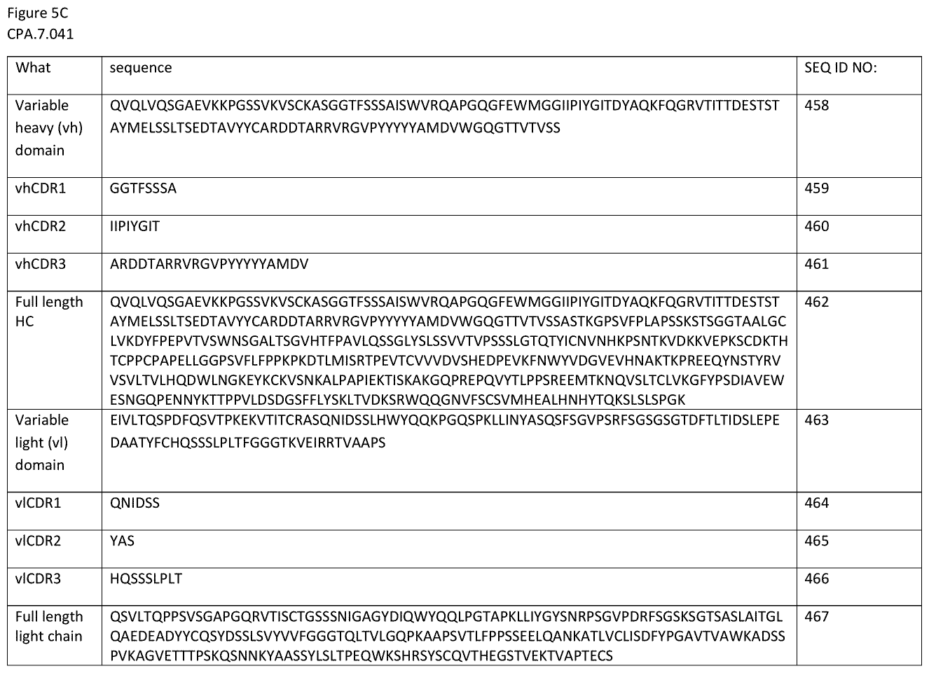

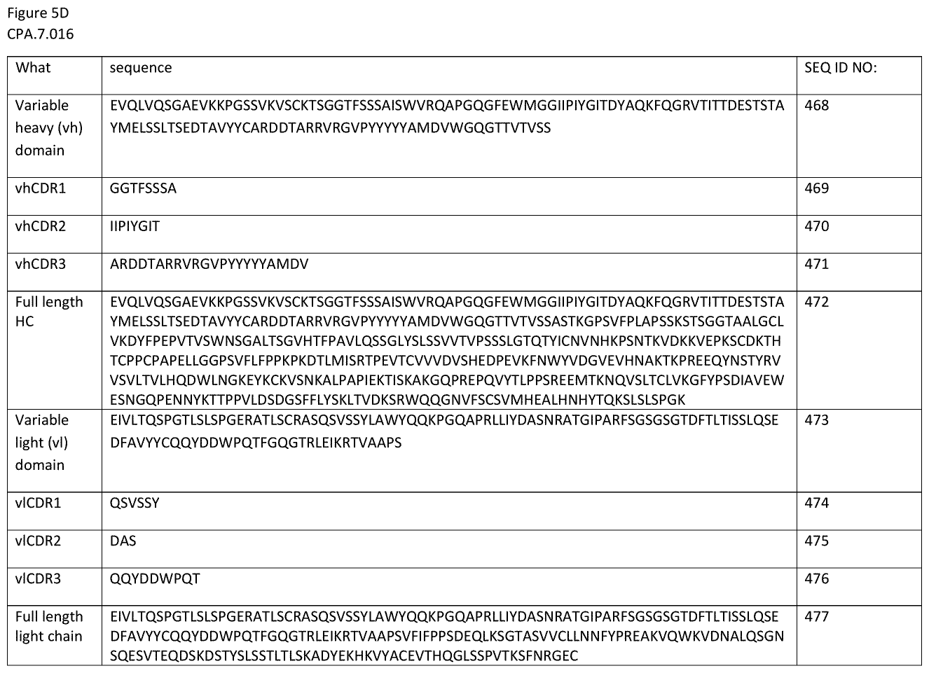

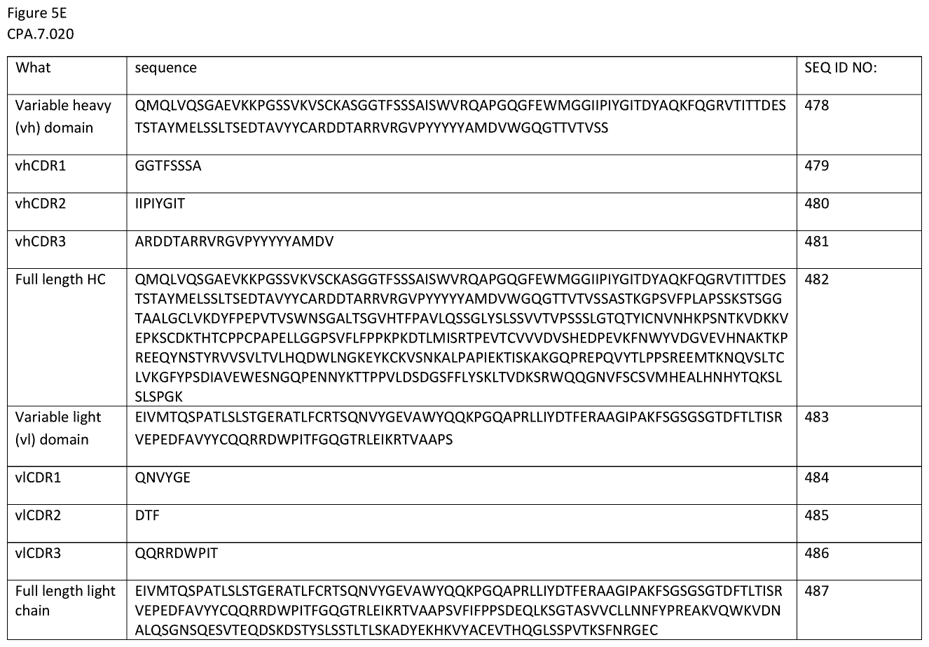

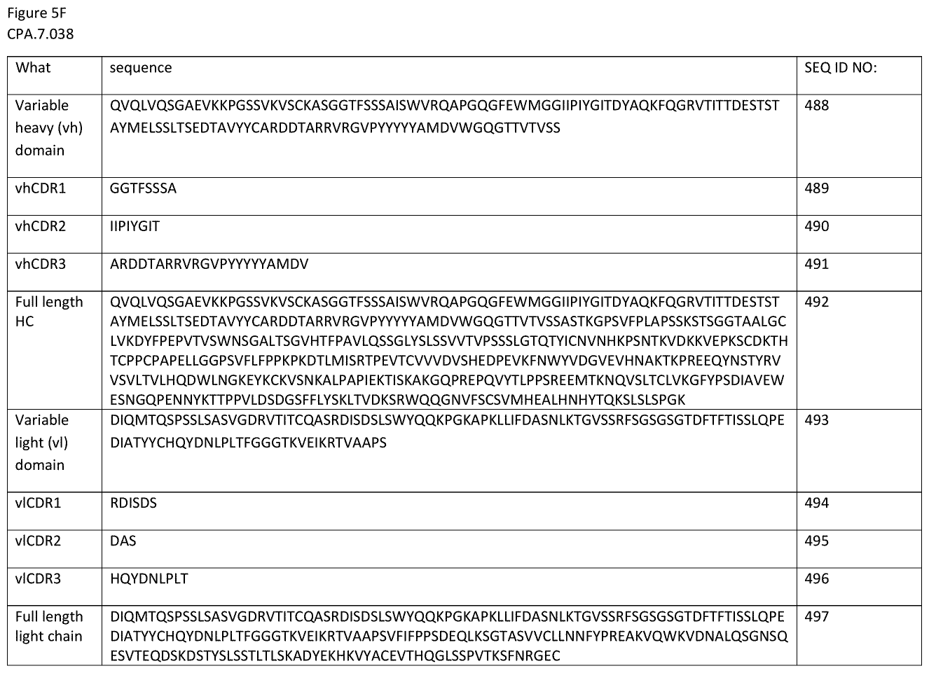

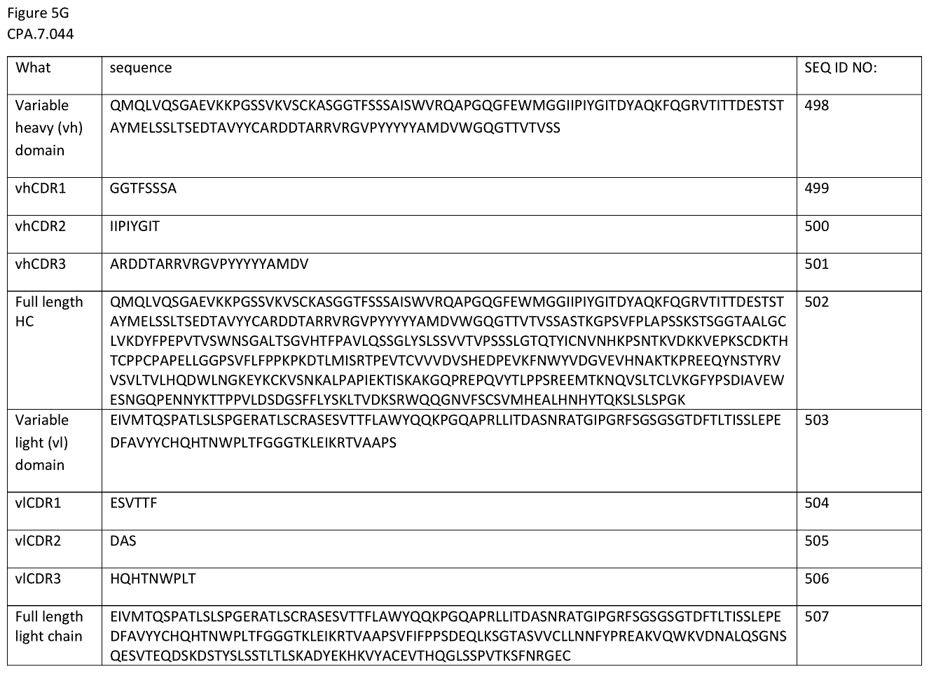

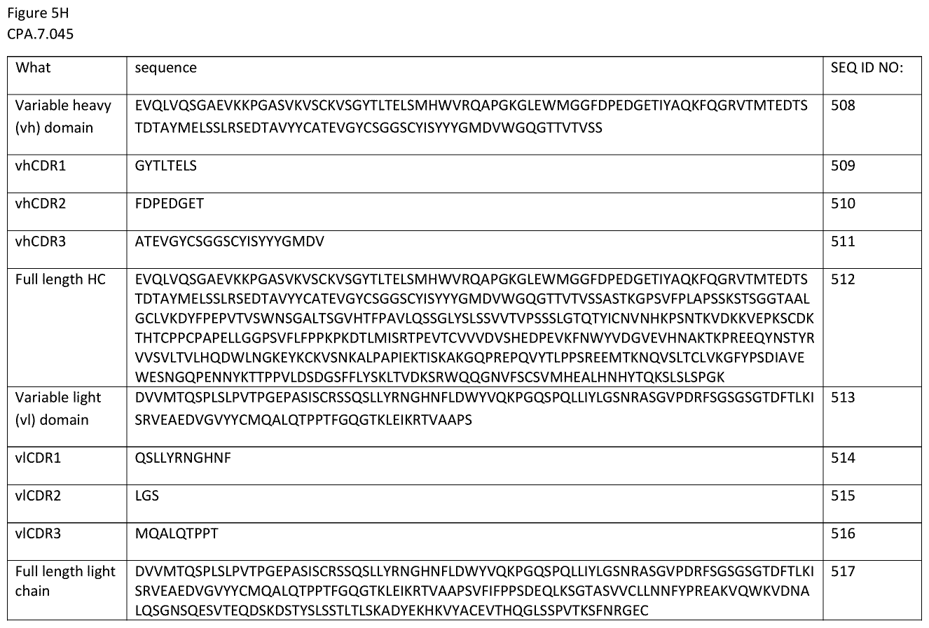

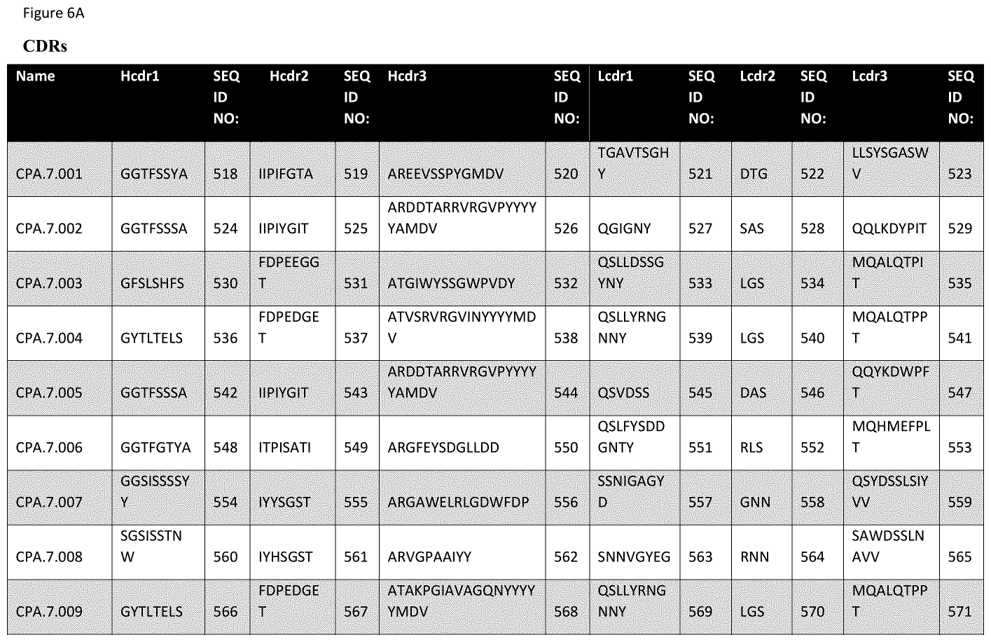

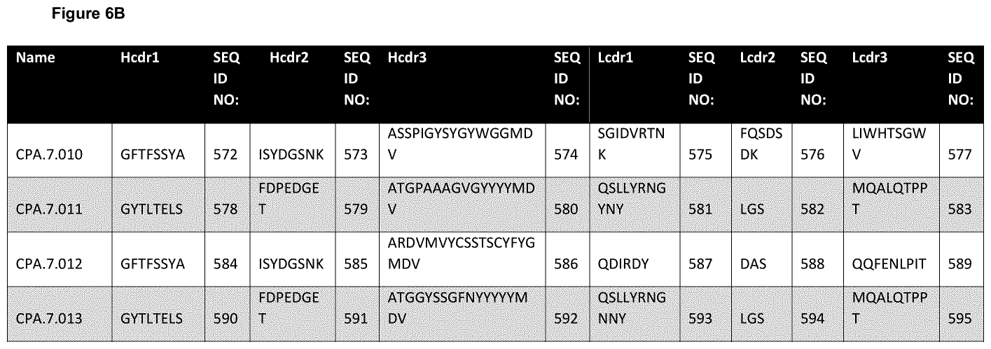

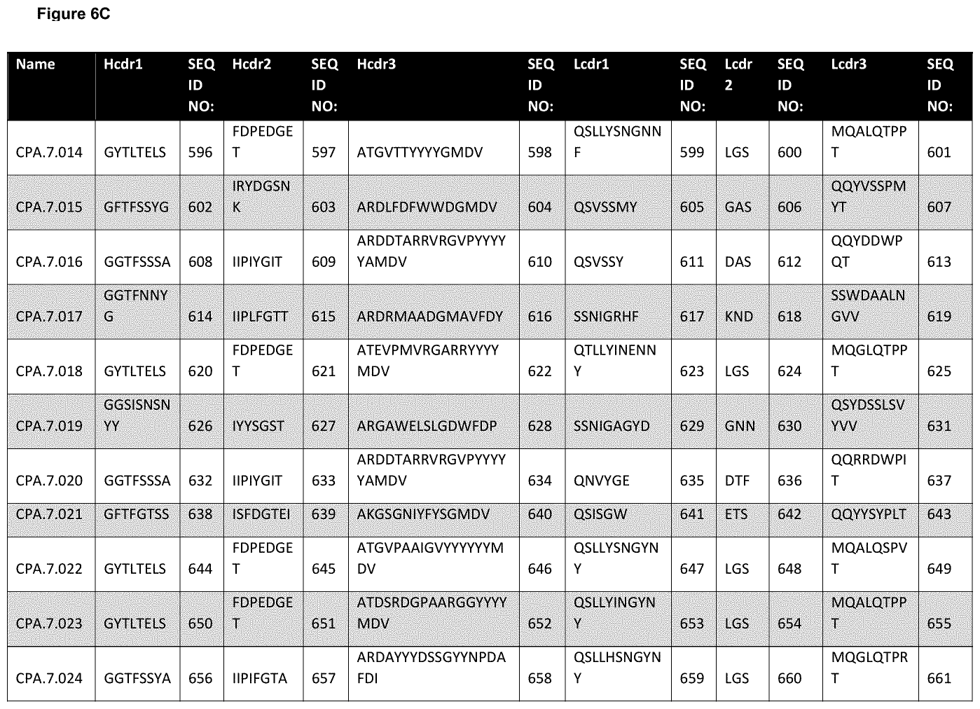

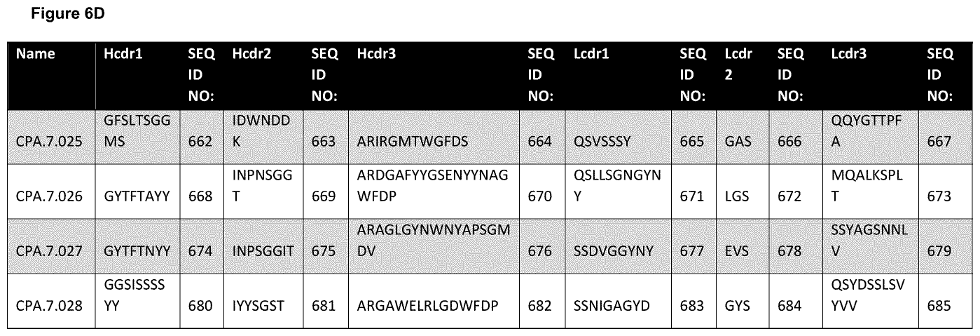

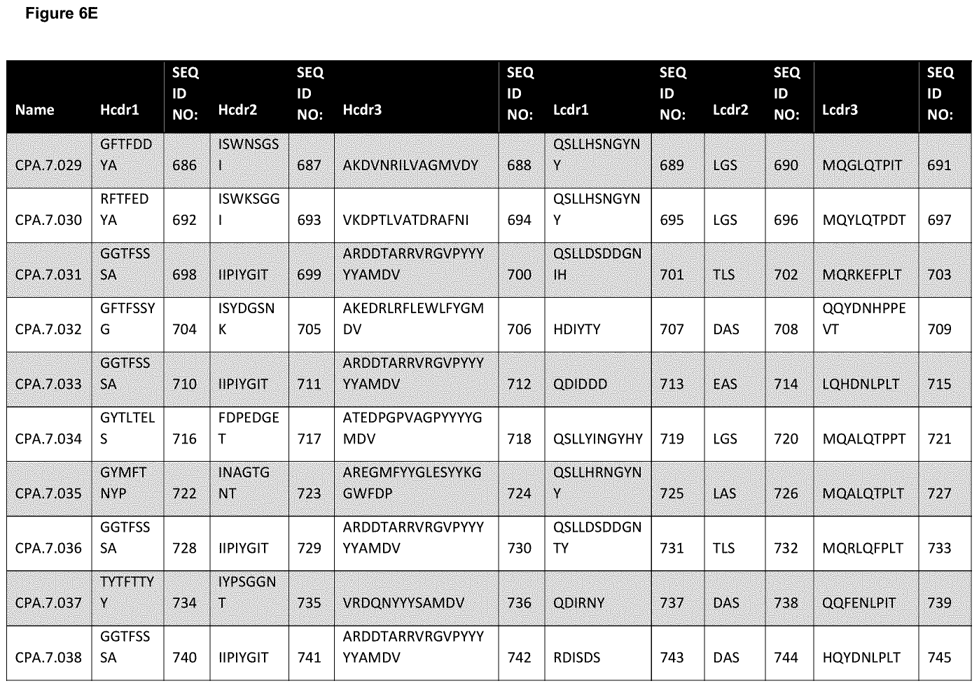

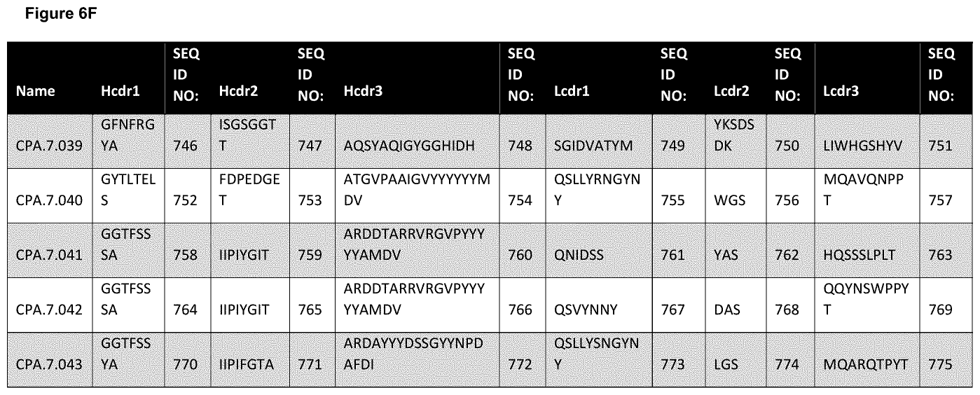

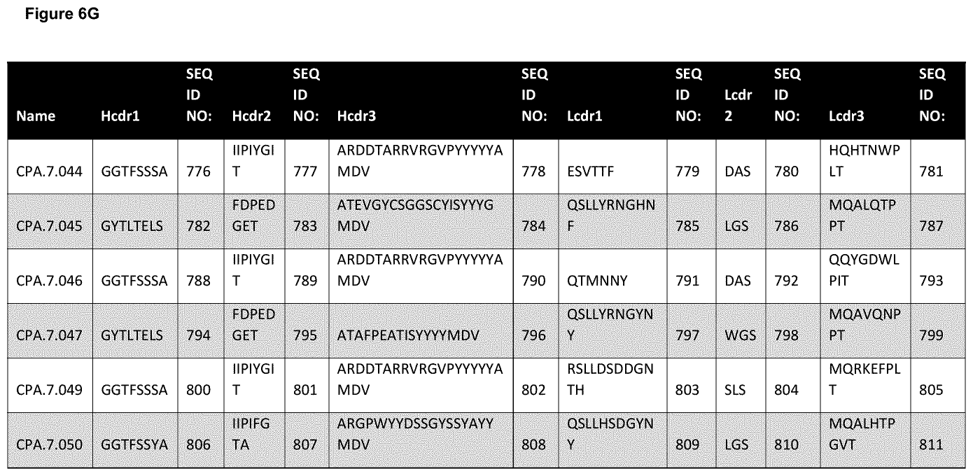

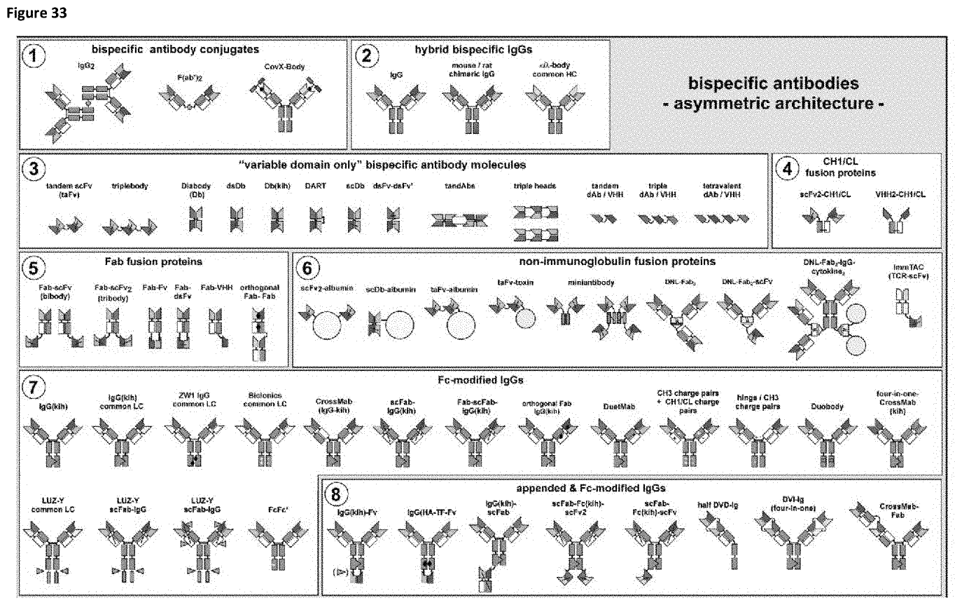

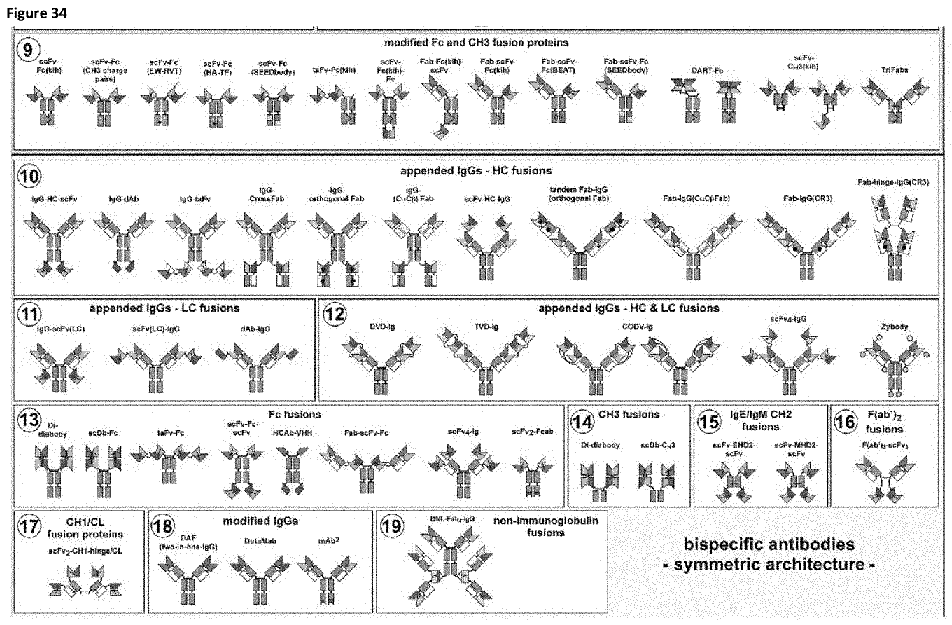

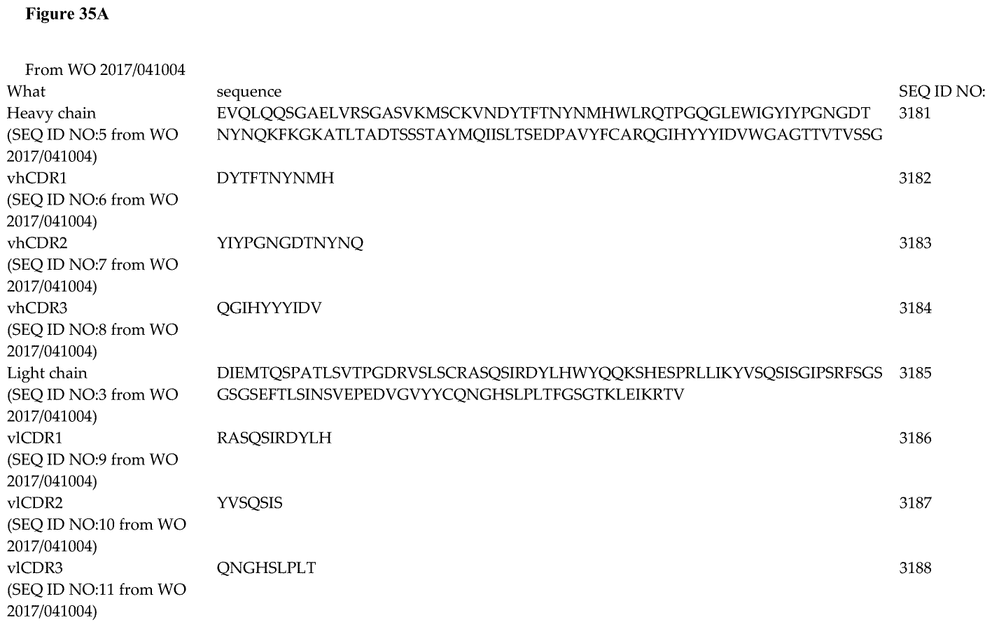

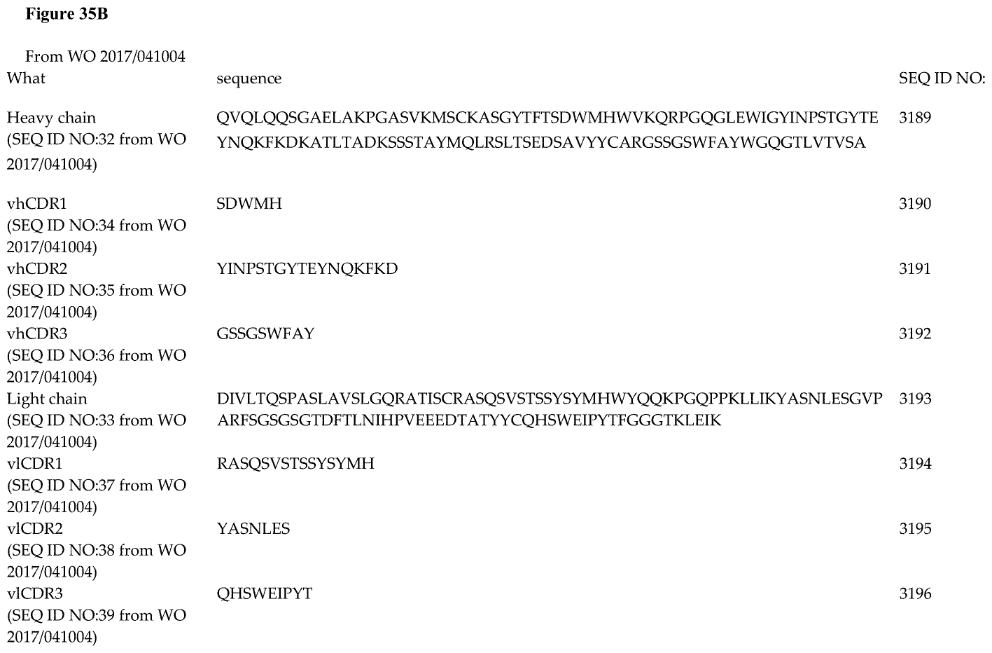

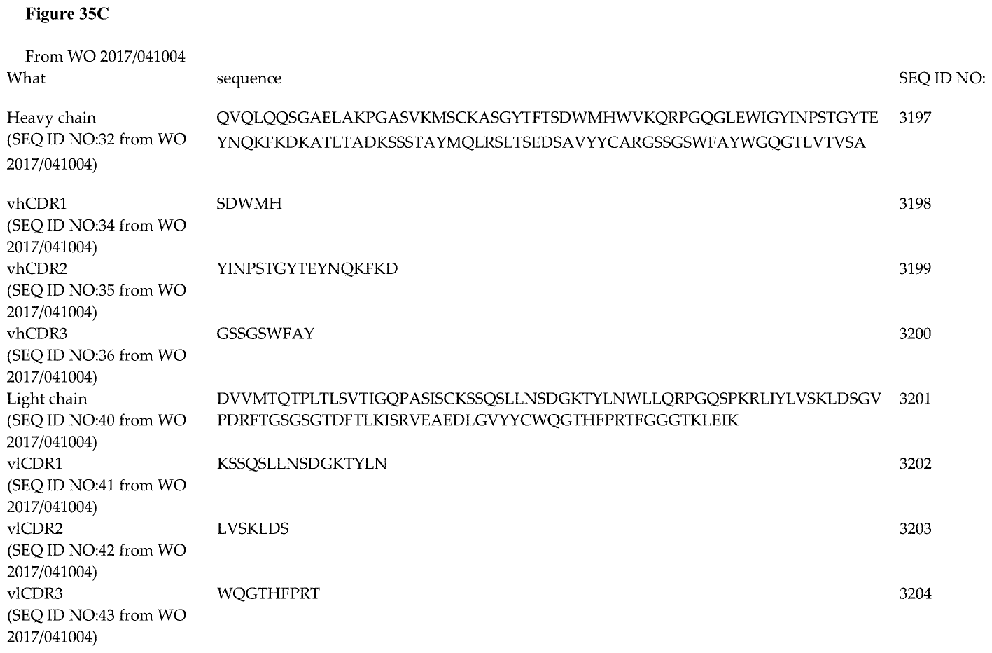

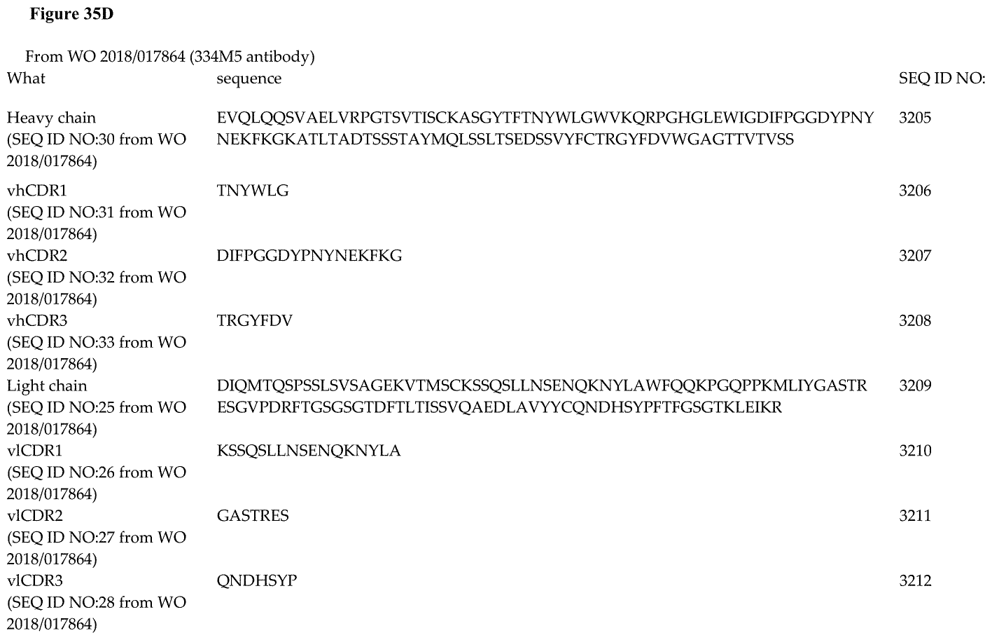

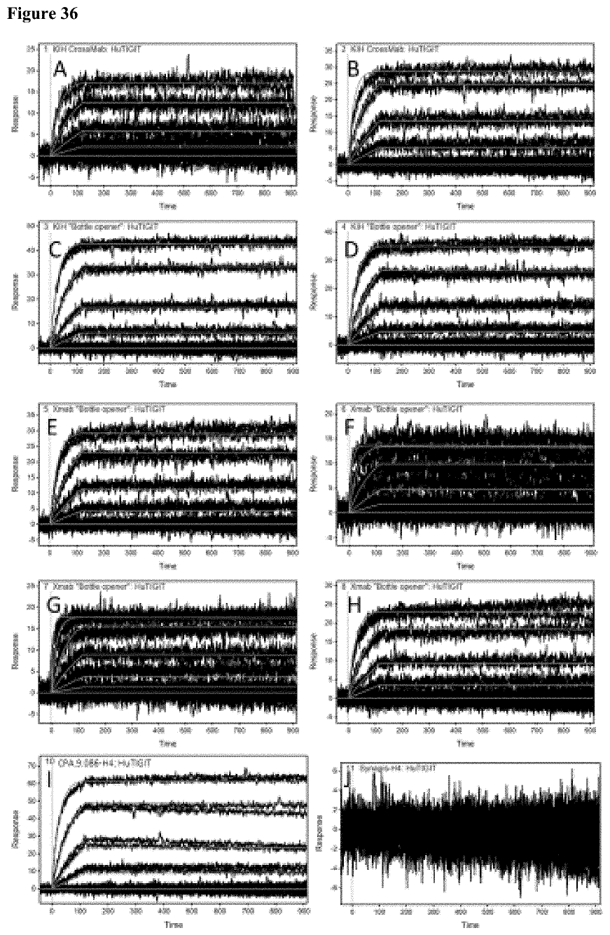

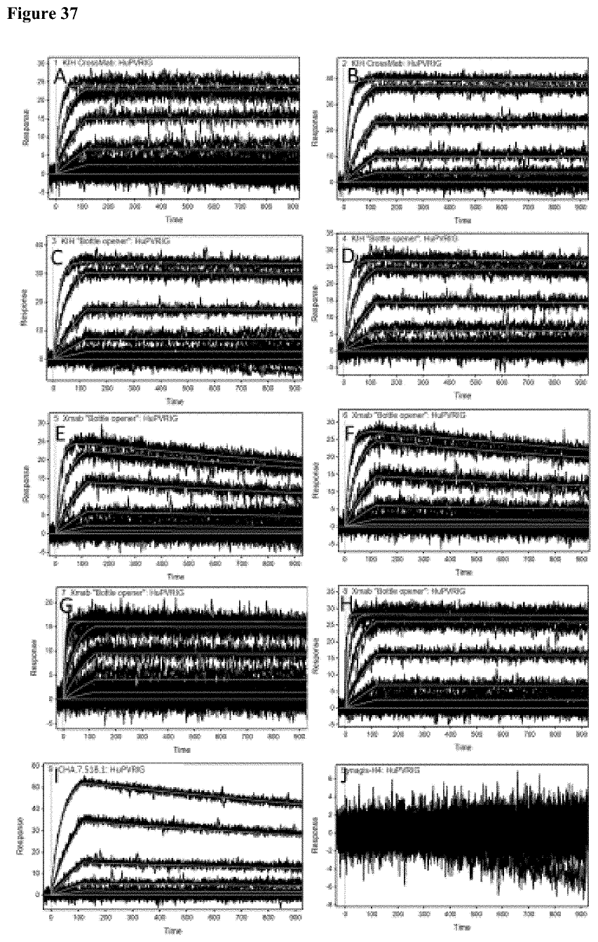

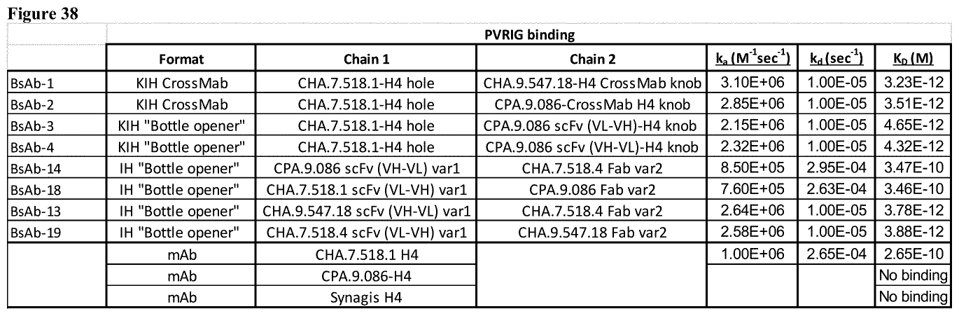

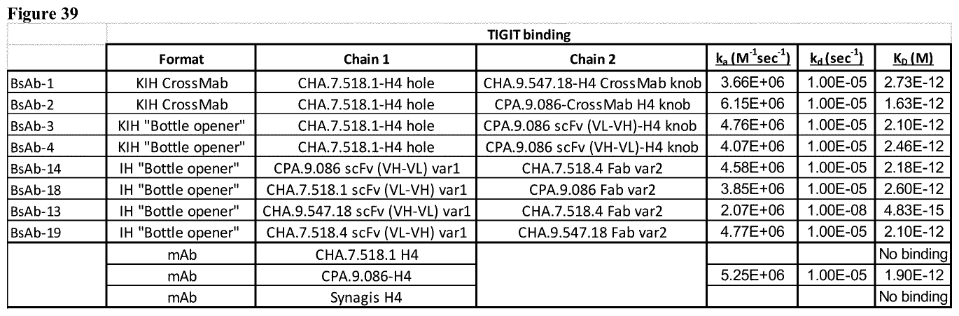

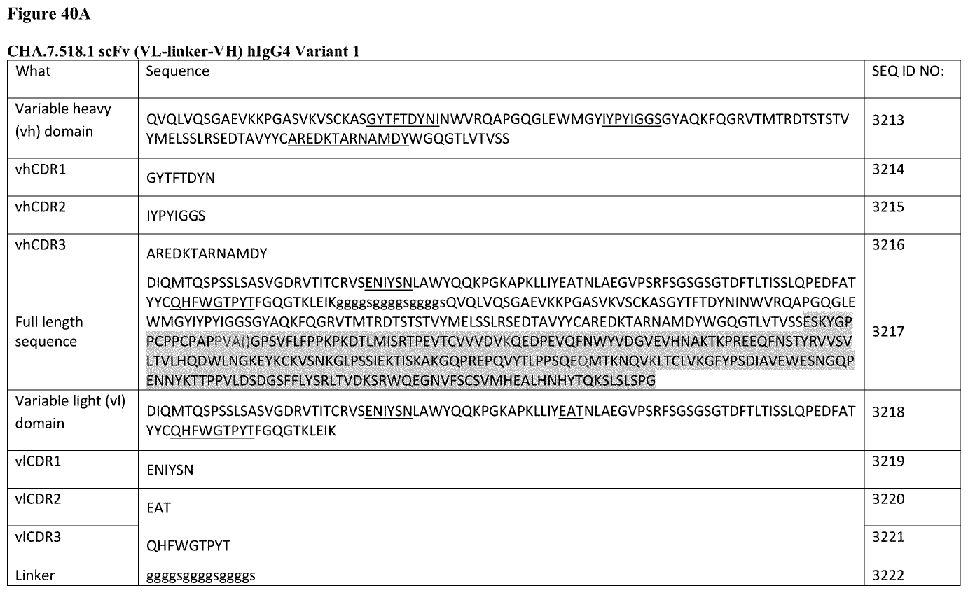

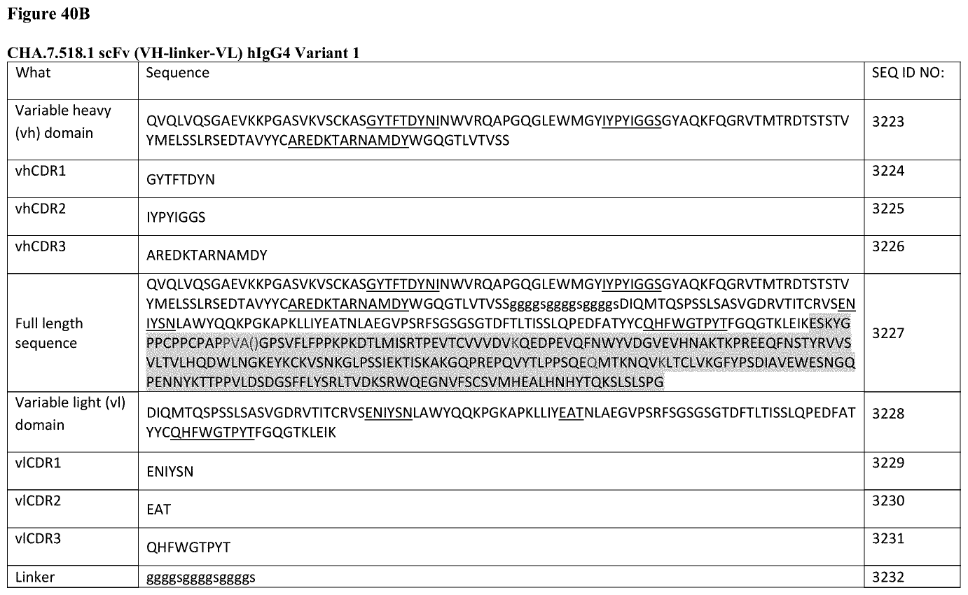

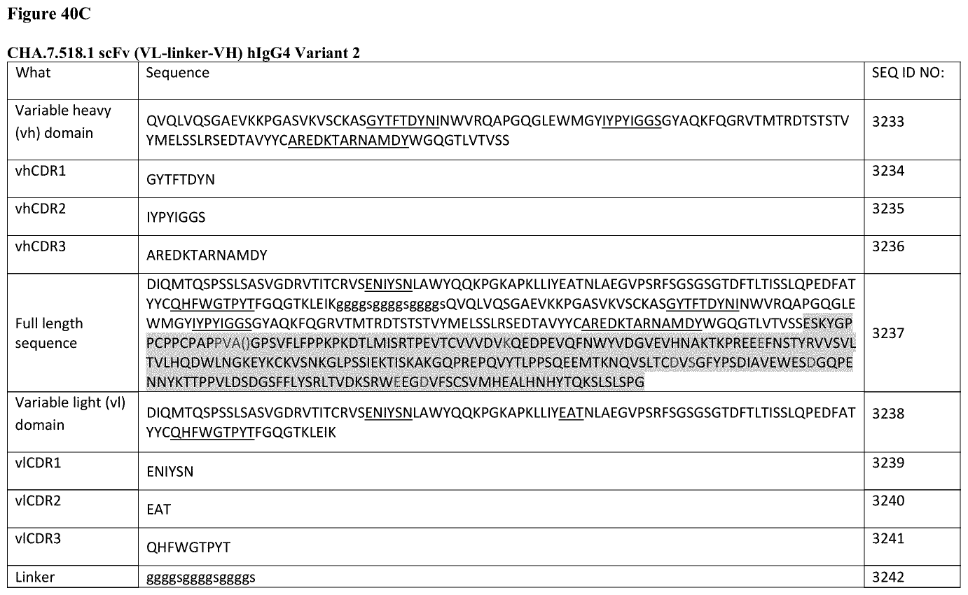

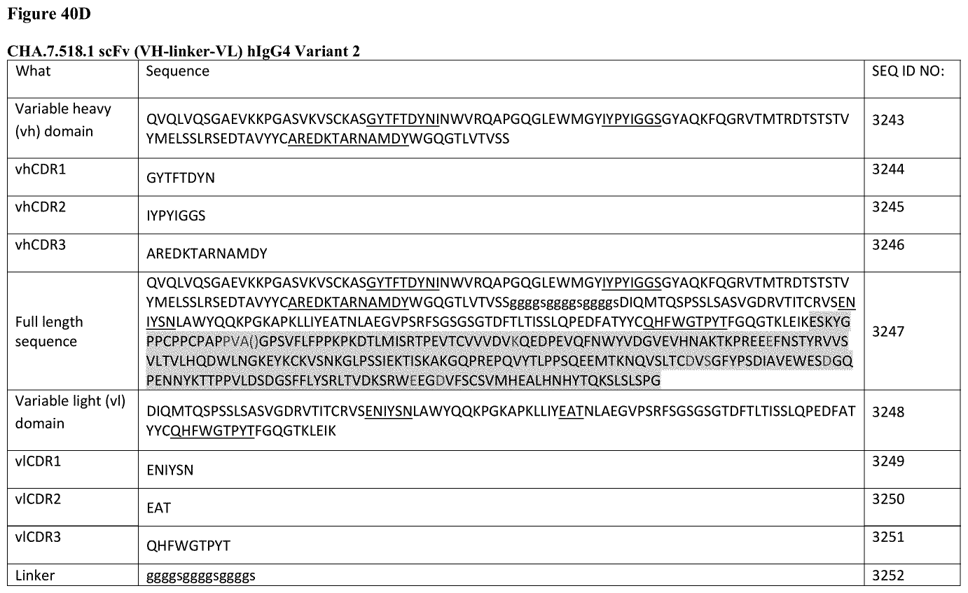

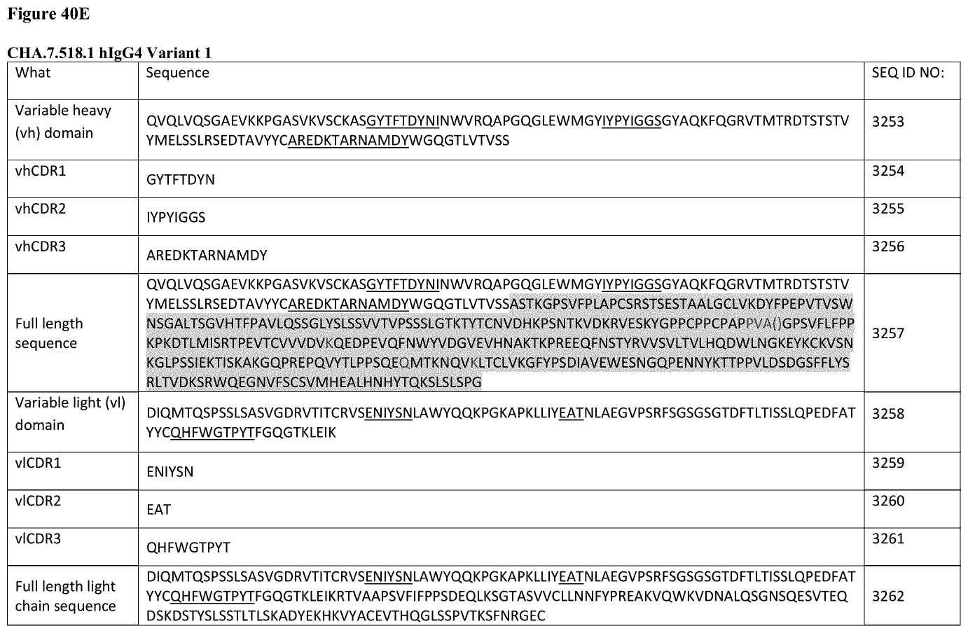

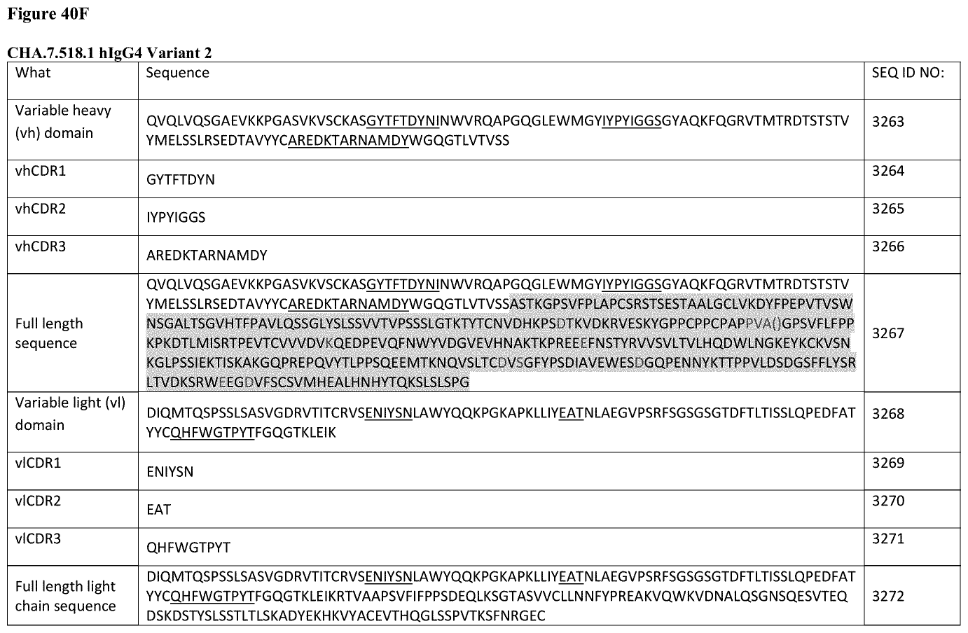

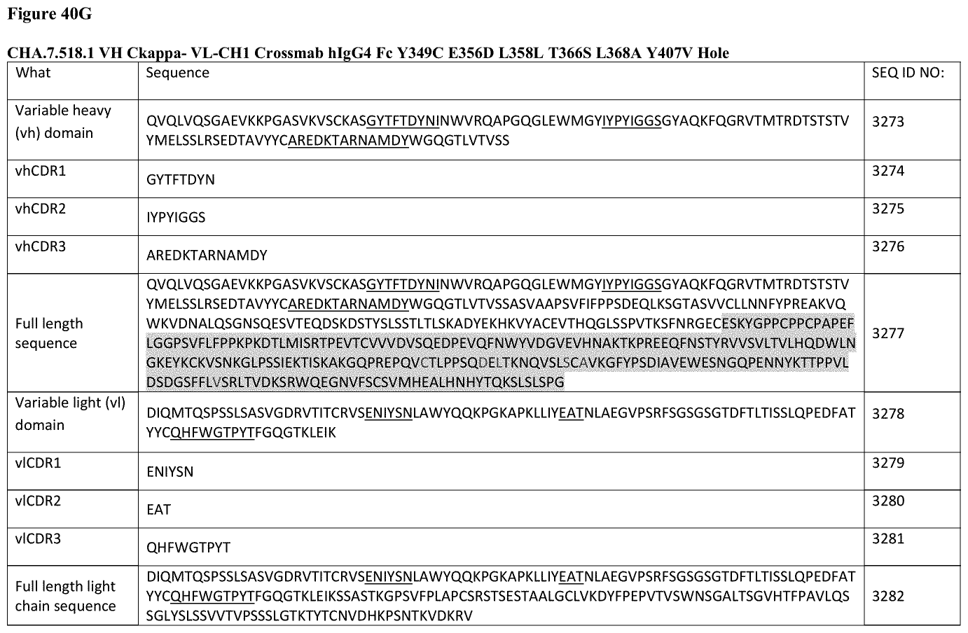

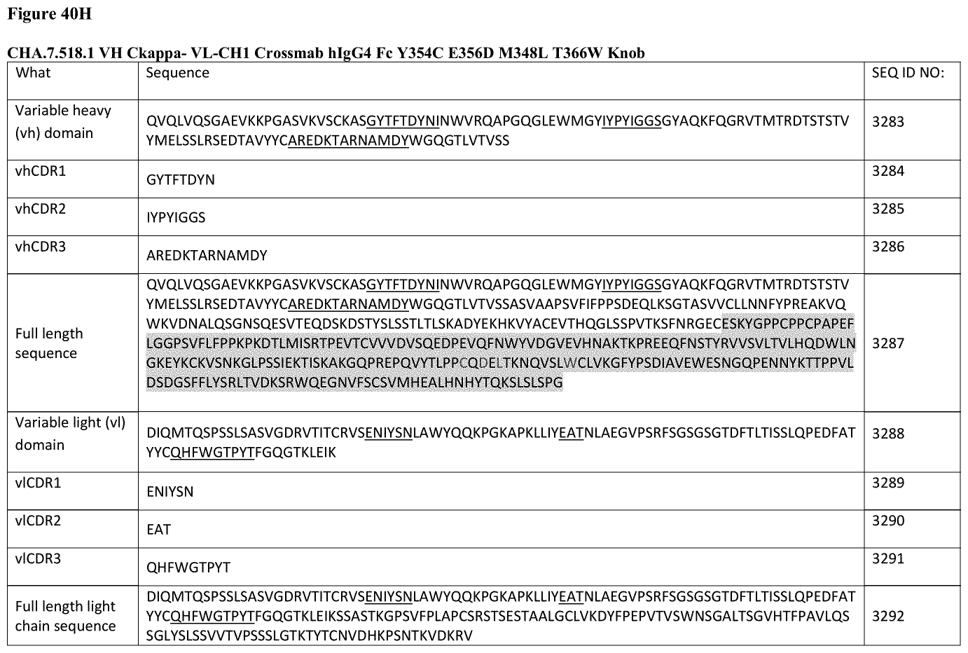

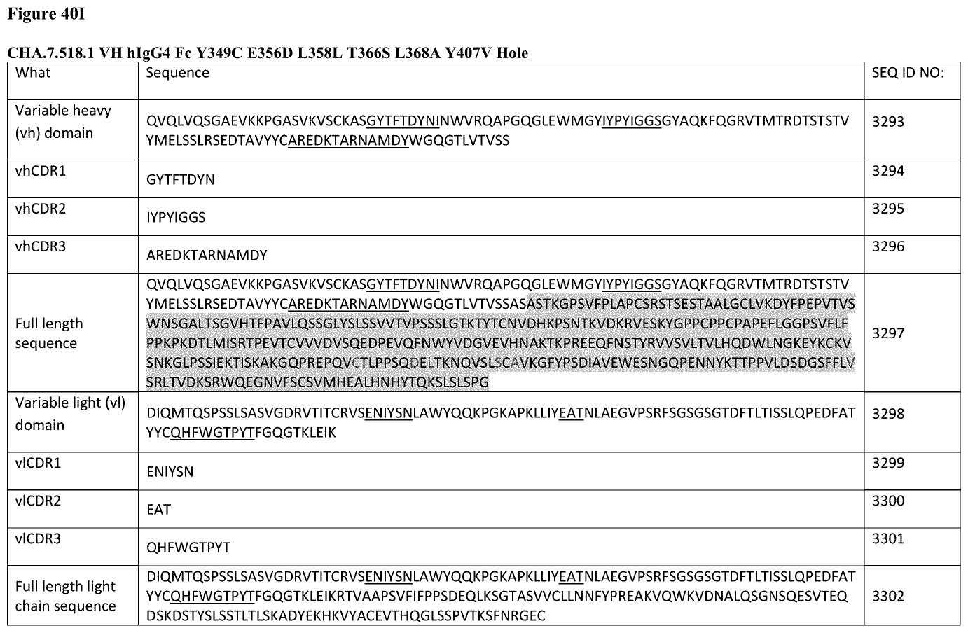

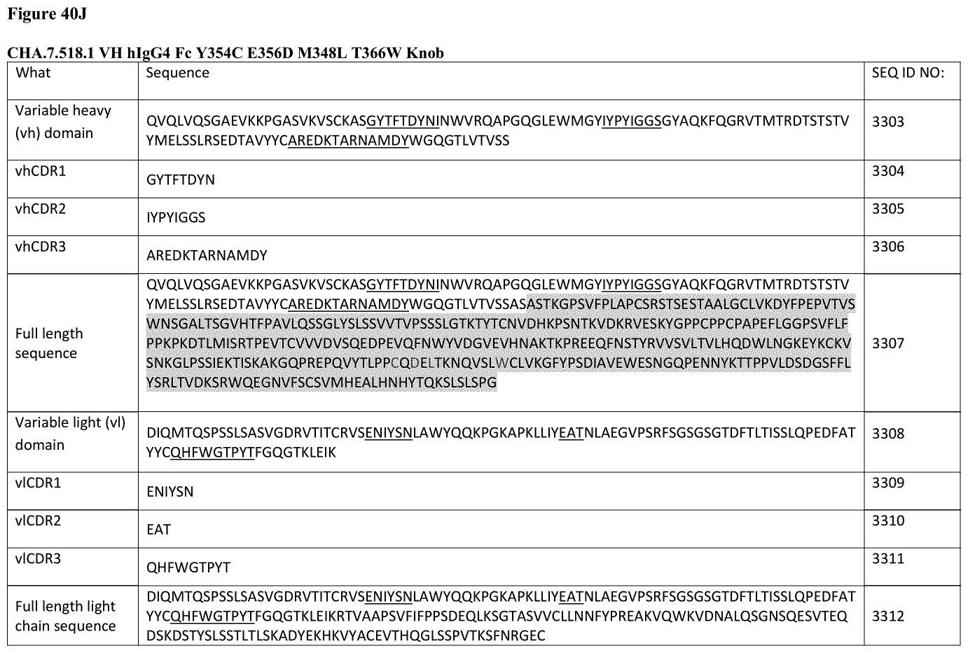

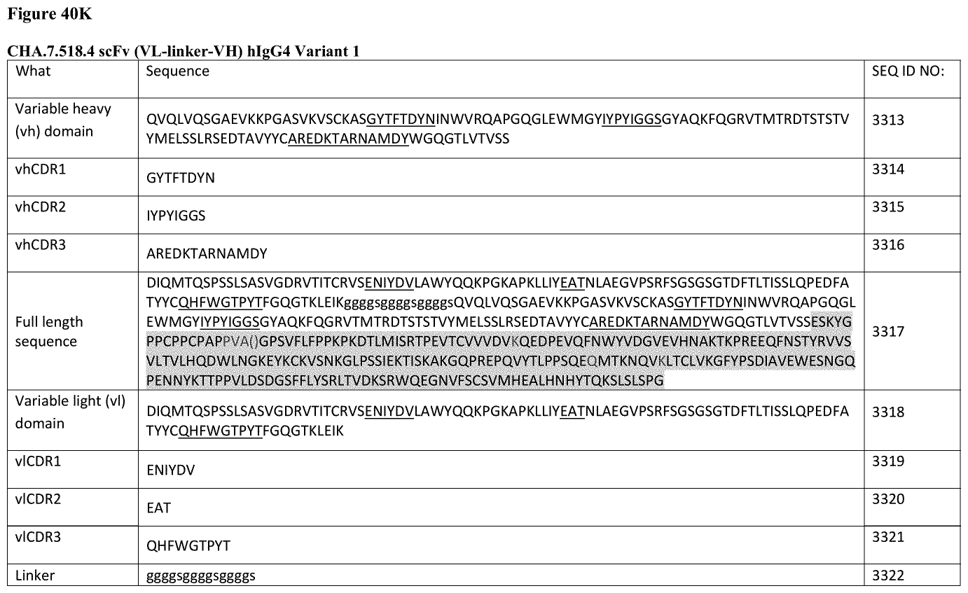

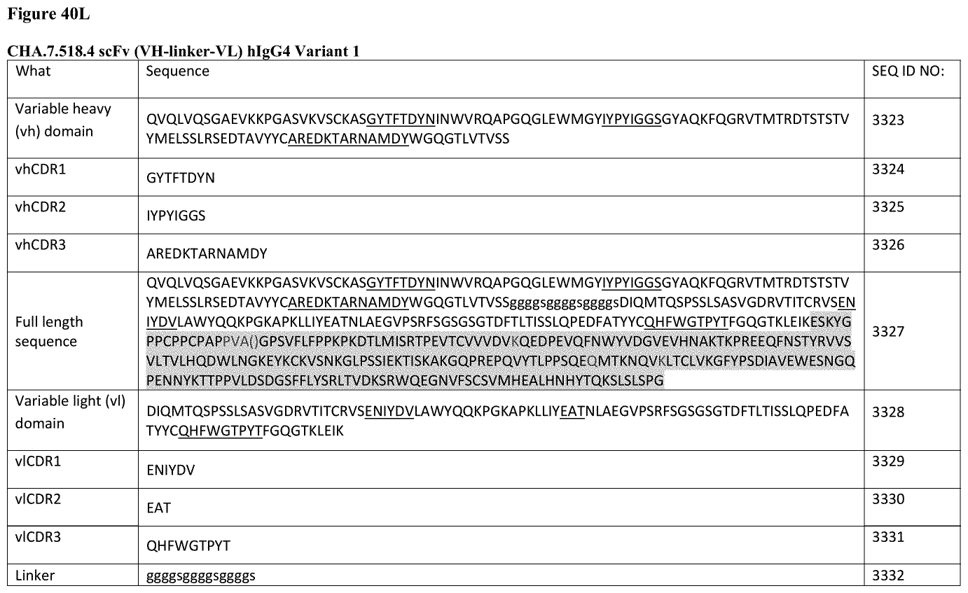

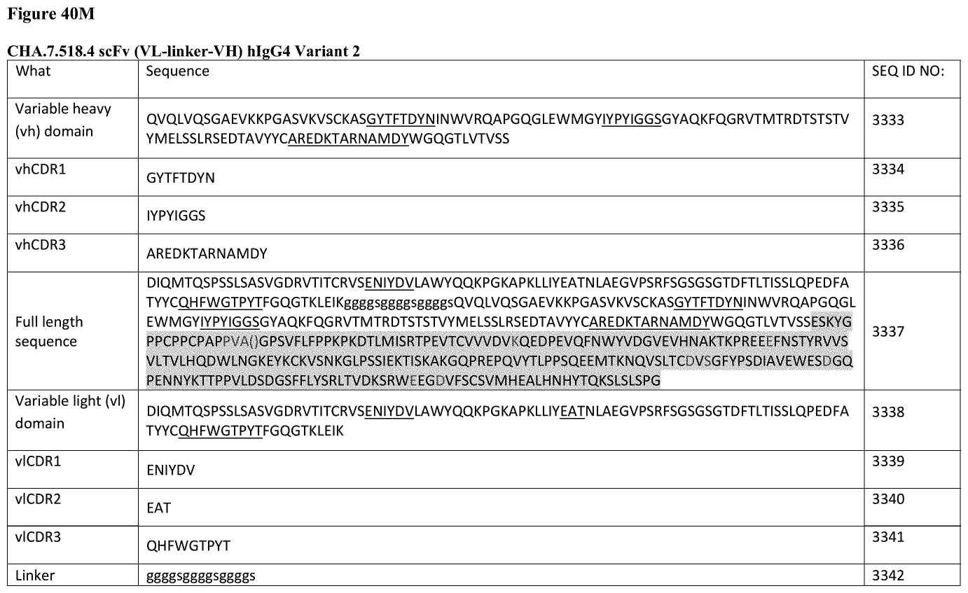

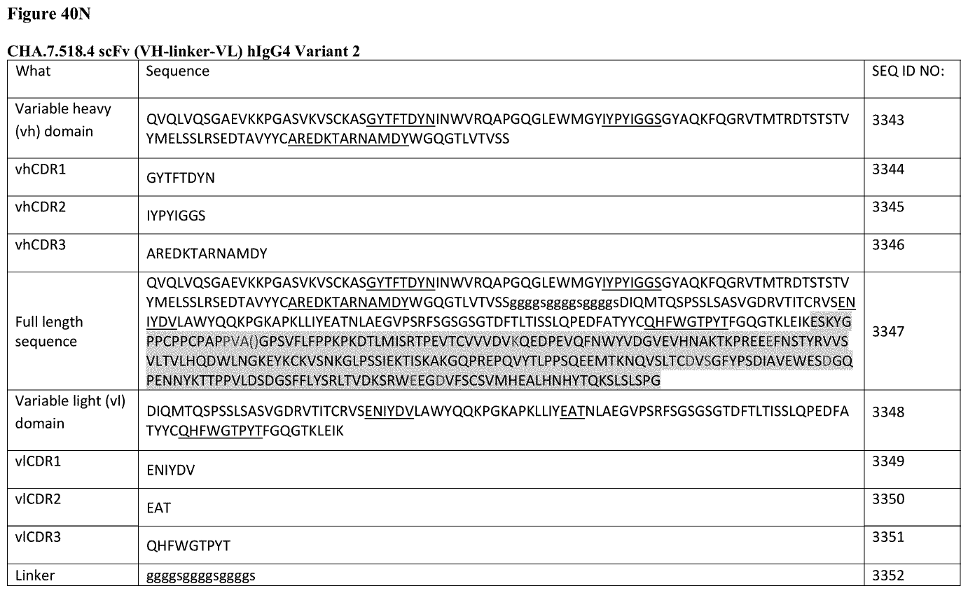

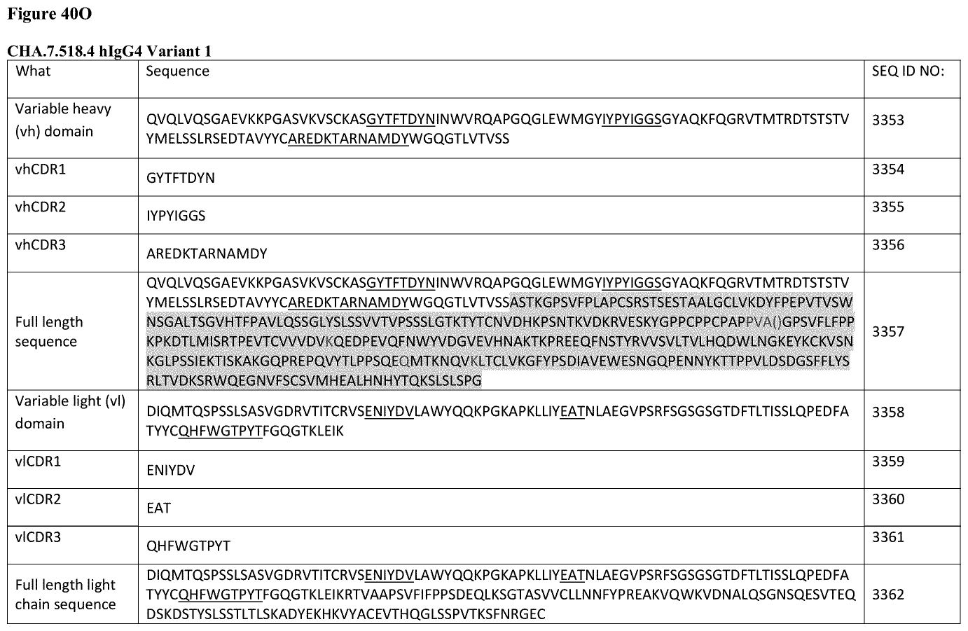

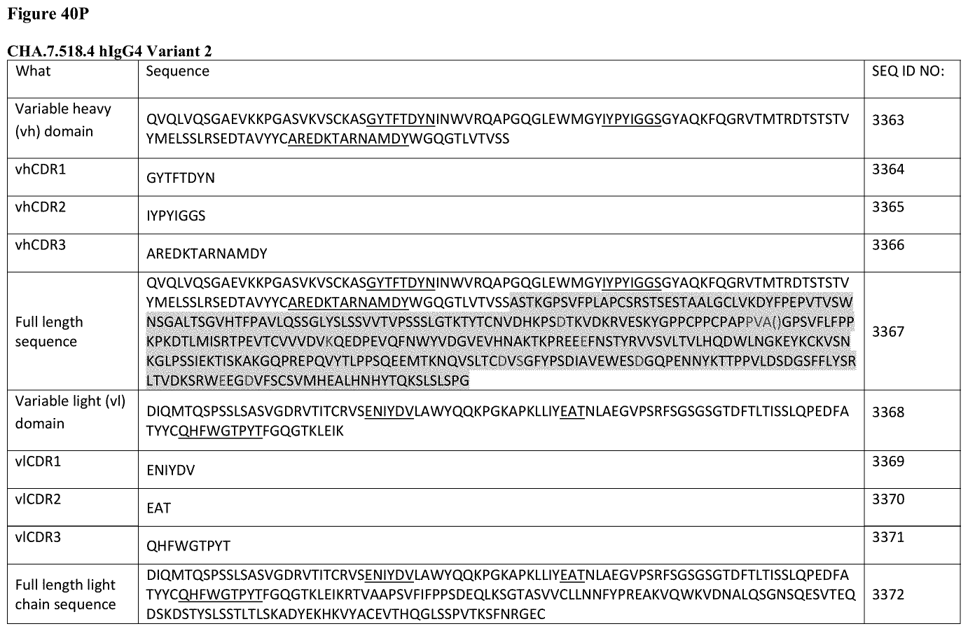

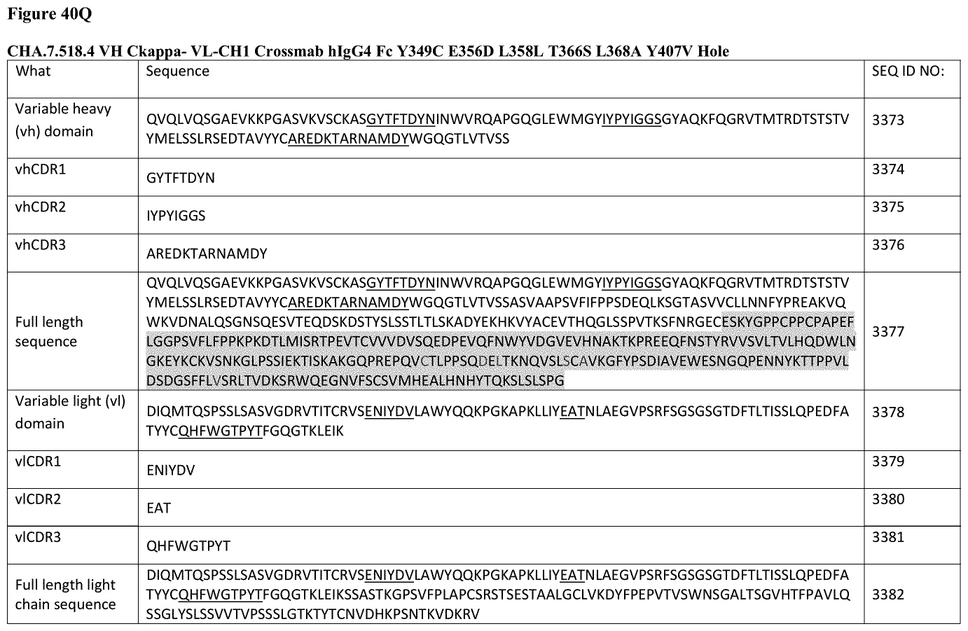

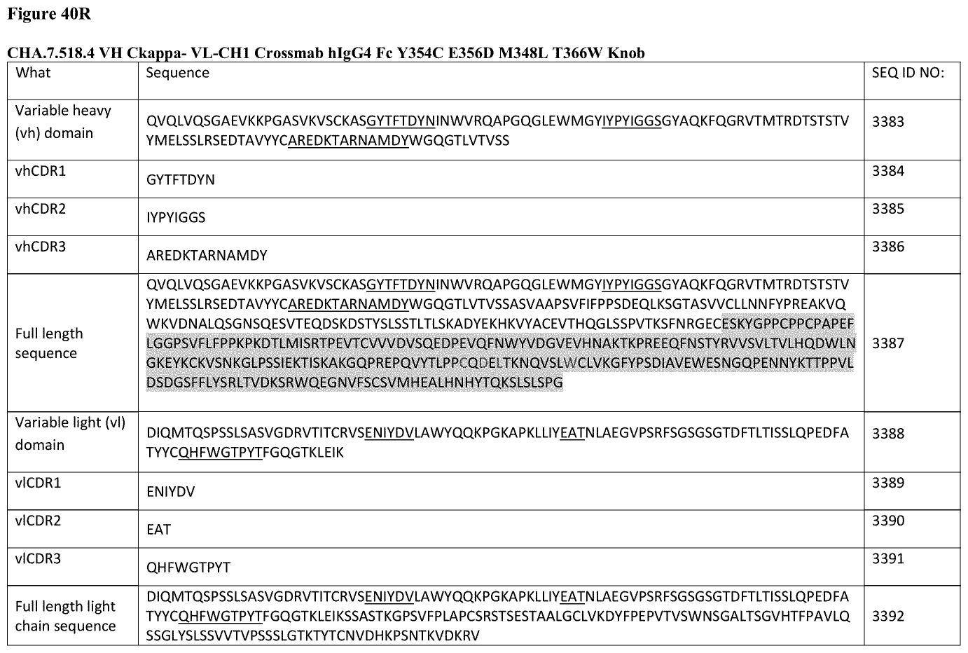

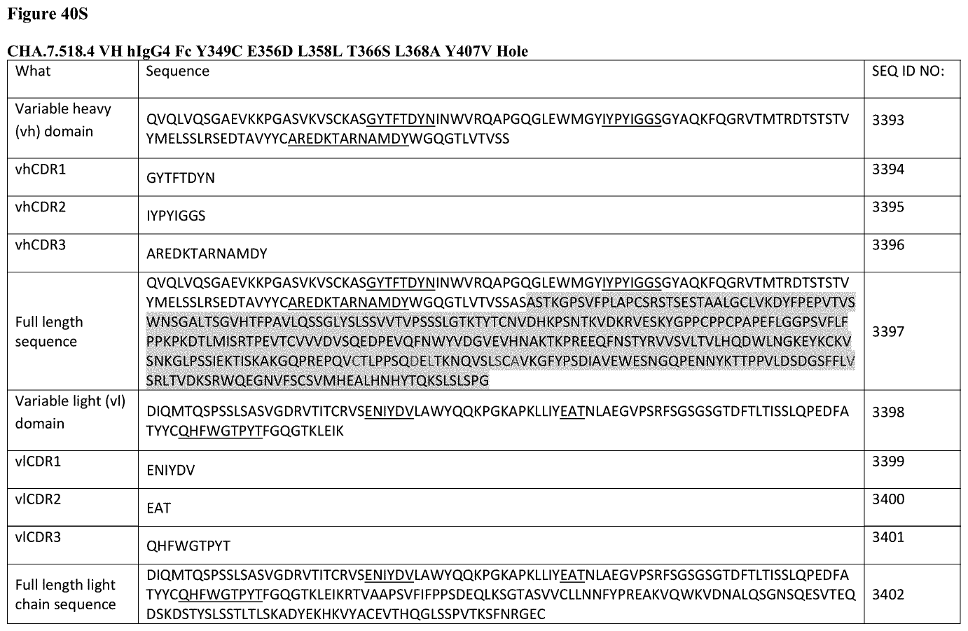

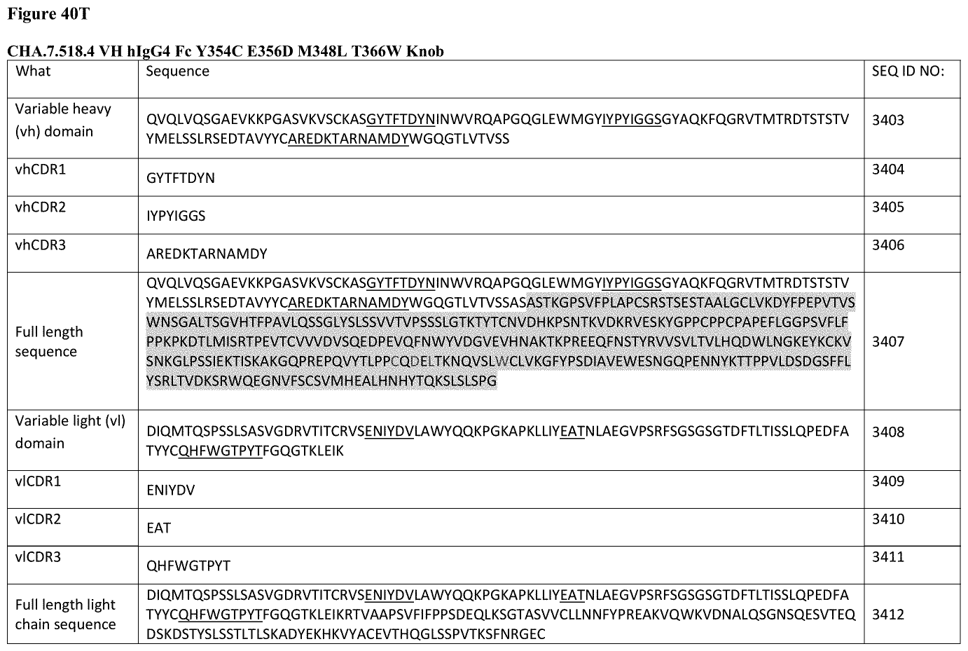

In some embodiments, binding affinity for the anti-PVRIG and/or anti-PVRIG/anti-TIGIT bispecific antibodies can be correlated with activity. Antibodies that exhibit the highest maximum signal on T cells can correlate with affinities in the picomolar range. In some embodiments, the anti-PVRIG/anti-TIGIT bispecific antibodies can be useful for T cell-based immunotherapy, which is based in part on their affinity. Reference is made to antibody sequences from WO2016/134333, hereby incorporated by reference and in particular for the anti-PVRIG antigen binding domains outlined in FIG. 38 (depicting sequences that bind PVRIG and block the interaction of PVRIG and PVRL2; include herein as FIG. 4A-4AA), FIG. 39 (depicting sequences that bind PVRIG and do not block the interaction of PVRIG and PVRL2; included herein as FIG. 5A-5H), FIG. 40A-40T (depicting CDRs and data from these antibodies; included herein as FIG. 6A-6G), and FIG. 41A-41T (depicting CDRs from hybridomas that bind and block; included herein as FIG. 7A-7AE), as well as FIG. 35A-35D providing additional PVRIG binding portions for use in the anti-PVRIG/anti-TIGIT bispecific antibodies. That is, the Figures and Legends as well as the particular sequences and SEQ ID NO:s from all CPA.7 and CHA.7 antibodies (including CDRs, VH and VL and full length sequences) from WO2016/134333 are expressly incorporated herein.

The anti-PVRIG and/or anti-PVRIG/anti-TIGIT bispecific antibodies of the invention have binding affinities (as measured using techniques outlined herein) in the picomolar range, e.g. from 0.1 to 9 pM, with from about 0.2 to about 2 being preferred, and from about 0.2 to about 0.5 being of particular use.

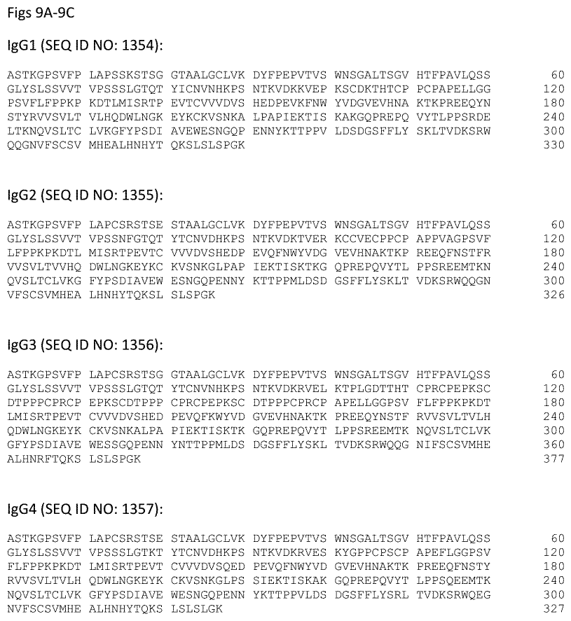

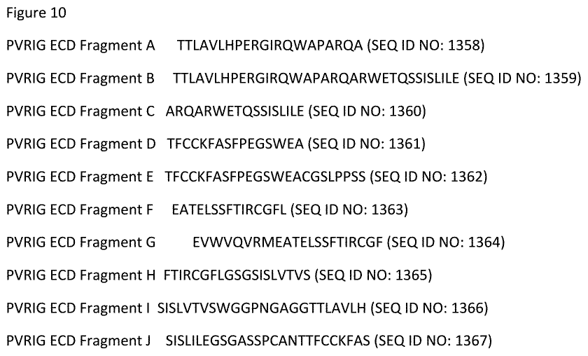

The PVRIG antibodies which can find use in providing the PVRIG binding portion of the anti-PVRIG/anti-TIGIT bispecific antibodies of the invention are labeled as follows. These PVRIG antibodies described herein are labeled as follows. The PVRIG antibodies have reference numbers, for example “CPA.7.013”. This represents the combination of the variable heavy and variable light chains, as depicted in FIG. 4A-4AA and FIGS. 5A-5H for example. “CPA.7.013.VH” refers to the variable heavy portion of CPA.7.013, while “CPA.7.013.VL” is the variable light chain. “CPA.7.013.vhCDR1”, “CPA.7.013.vhCDR2”, “CPA.7.013.vhCDR3”, “CPA.7.013.vlCDR1”, “CPA.7.013.vlCDR2”, and “CPA.7.013.vlCDR3”, refers to the CDRs are indicated. “CPA.7.013.HC” refers to the entire heavy chain (e.g. variable and constant domain) of this molecule, and “CPA.7.013.LC” refers to the entire light light chain (e.g. variable and constant domain) of the same molecule. “CPA.7.013.H1” refers to a full length antibody comprising the variable heavy and light domains, including the constant domain of Human IgG1 (hence, the H1; IgG1, IgG2, IgG3 and IgG4 sequences are shown in FIGS. 9A-9C and 21A-21B). Accordingly, “CPA.7.013.H2” would be the CPA.7.013 variable domains linked to a Human IgG2. “CPA.7.013.H3” would be the CPA.7.013 variable domains linked to a Human IgG3, and “CPA.7.013.H4” would be the CPA.7.013 variable domains linked to a Human IgG4. The anti-PVRIG/anti-TIGIT bispecific antibodies of the invention can comprise any of the PVRIG antibody sequences and/or PVRIG antigen binding domain sequences as the PVRIG binding portion of the anti-PVRIG/anti-TIGIT bispecific antibodies.

The PVRIG antibodies which can find use in providing the PVRIG binding portion of the anti-PVRIG/anti-TIGIT bispecific antibodies of the invention are labeled as follows. The antibodies have reference numbers, for example “CHA.7.518.1”. This represents the combination of the variable heavy and variable light chains, as depicted in FIG. 7A-7AE, for example, with the understanding that these antibodies include two heavy chains and two light chains. “CPA. 7.518.1.VH” refers to the variable heavy portion of CPA. 7.518.1, while “CPA.7.518.1.VL” is the variable light chain. “CPA. 7.518.1.vhCDR1”, “CPA.7.518.1.vhCDR2”, “CPA. 7.518.1.vhCDR3”, “CPA. 7.518.1.vlCDR1”, “CPA. 7.518.1.vlCDR2”, and “CPA. 7.518.1.vlCDR3”, refers to the CDRs are indicated. “CPA. 7.518.1.HC” refers to the entire heavy chain (e.g. variable and constant domain) of this molecule, and “CPA. 7.518.1.LC” refers to the entire light chain (e.g. variable and constant domain) of the same molecule. In general, the human kappa light chain is used for the constant domain of each phage (or humanized hybridoma) antibody herein, although in some embodiments the lambda light constant domain is used. “CPA. 7.518.1.H1” refers to a full-length antibody comprising the variable heavy and light domains, including the constant domain of Human IgG1 (hence, the H1; IgG1, IgG2, IgG3 and IgG4 sequences are shown in FIG. 21A-21B). Accordingly, “CPA. 7.518.1.H2” would be the CPA. 7.518.1 variable domains linked to a Human IgG2. “CPA. 7.518.1.H3” would be the CPA. 7.518.1 variable domains linked to a Human IgG3, and “CPA. 7.518.1.H4” would be the CPA. 7.518.1 variable domains linked to a Human IgG4. Note that in some cases, the human IgGs may have additional mutations, such are described below, and this can be annotated. For example, in many embodiments, there may be a S241P mutation in the human IgG4, and this can be annotated as “CPA. 7.518.1.H4(S241P)” for example. The human IgG4 sequence with this S241P hinge variant is shown in FIG. 21A-21B. Other potential variants are IgG1(N297A), (or other variants that ablate glycosylation at this site and thus many of the effector functions associated with FcγRIIIa binding), and IgG1(D265A), which reduces binding to FcγR receptors. The anti-PVRIG/anti-TIGIT bispecific antibodies of the invention can comprise any of the PVRIG antibody sequences as the PVRIG binding portion of the anti-PVRIG/anti-TIGIT bispecific antibodies. The anti-PVRIG/anti-TIGIT bispecific antibodies of the invention can comprise any of the PVRIG antigen binding domain sequences as the PVRIG binding portion of the anti-PVRIG/anti-TIGIT bispecific antibodies.

The invention further provides variable heavy and light domains as well as full length heavy and light chains, any of which can be employed as part of the PVRIG binding portion of the anti-PVRIG/anti-TIGIT bispecific antibodies.

In some embodiments, the invention provides scFvs that bind to PVRIG comprising a variable heavy domain and a variable light domain linked by an scFv linker as outlined above. The VL and VH domains can be in either orientation, e.g. from N- to C-terminus “VH-linker-VL” or “VL-linker”VH”. These are named by their component parts; for example, “scFv-CHA.7.518.1VH-linker-VL” or “scFv-CPA. 7.518.1.VL-linker-VH.” Thus, “scFv-CPA. 7.518.1” can be in either orientation. The anti-PVRIG/anti-TIGIT bispecific antibodies of the invention can comprise an scFv that binds to PVRIG as the PVRIG binding portion of the anti-PVRIG/anti-TIGIT bispecific antibodies.

The invention provides antigen binding domains, including full length antibodies, which contain a number of specific, enumerated sets of 6 CDRs. The anti-PVRIG/anti-TIGIT bispecific antibodies of the invention can comprise any of the sets of 6 CDRs from the PVRIG antibody sequences provided herein in the PVRIG binding portion of the anti-PVRIG/anti-TIGIT bispecific antibodies.

The invention further provides variable heavy and light domains as well as full length heavy and light chains.

In many embodiments, the anti-PVRIG/anti-TIGIT bispecific antibodies of the invention are human (derived from phage) and block binding of PVRIG and PVLR2. The anti-PVRIG/anti-TIGIT bispecific antibodies of the invention can comprise a PVRIG antibody and/or antigen binding domain sequence capable of both binding and blocking the receptor-ligand interaction as the PVRIG binding portion of the anti-PVRIG/anti-TIGIT bispecific antibodies. The anti-PVRIG/anti-TIGIT bispecific antibodies of the invention can comprise the CDRs from a PVRIG antibody sequence capable of both binding and blocking the receptor-ligand interaction as the PVRIG binding portion of the anti-PVRIG/anti-TIGIT bispecific antibodies. The CPA antibodies, as well as the CDR sequences, that both bind and block the receptor-ligand interaction are as below, with their components outlined as well, the sequences for which are shown in FIG. 4A-4AA:

CPA.7.001, CPA.7.001.VH, CPA.7.001.VL, CPA.7.001.HC, CPA.7.001.LC and CPA.7.001.H1, CPA.7.001.H2, CPA.7.001.H3, CPA.7.001.H4; CPA.7.001.vhCDR1, CPA.7.001.vhCDR2, CPA.7.001.vhCDR3, CPA.7.001.vlCDR1, CPA.7.001.vlCDR2, and CPA.7.001.vlCDR3;

CPA.7.003, CPA.7.003.VH, CPA.7.003.VL, CPA.7.003.HC, CPA.7.003.LC, CPA.7.003.H1, CPA.7.003.H2, CPA.7.003.H3, CPA.7.003.H4; CPA.7.003.vhCDR1, CPA.7.003.vhCDR2, CPA.7.003.vhCDR3, CPA.7.003.vlCDR1, CPA.7.003.vlCDR2, and CPA.7.003.vlCDR3;

CPA.7.004, CPA.7.004.VH, CPA.7.004.VL, CPA.7.004.HC, CPA.7.004.LC, CPA.7.004.H1, CPA.7.004.H2, CPA.7.004.H3 CPA.7.004.H4; CPA.7.004.vhCDR1, CPA.7.004.vhCDR2, CPA.7.004.vhCDR3, CPA.7.004.vlCDR1, CPA.7.004.vlCDR2, and CPA.7.004.vlCDR3;

CPA.7.006, CPA.7.006.VH, CPA.7.006.VL, CPA.7.006.HC, CPA.7.006.LC, CPA.7.006.H1, CPA.7.006.H2, CPA.7.006.H3 CPA.7.006.H4; CPA.7.006.vhCDR1, CPA.7.006.vhCDR2, CPA.7.006.vhCDR3, CPA.7.006.vlCDR1, CPA.7.006.vlCDR2, and CPA.7.006.vlCDR3;

CPA.7.008, CPA.7.008.VH, CPA.7.008.VL, CPA.7.008.HC, CPA.7.008.LC, CPA.7.008.H1, CPA.7.008.H2, CPA.7.008.H3 CPA.7.008.H4; CPA.7.008.vhCDR1, CPA.7.008.vhCDR2, CPA.7.008.vhCDR3, CPA.7.008.vlCDR1, CPA.7.008.vlCDR2, and CPA.7.008.vlCDR3;

CPA.7.009, CPA.7.009.VH, CPA.7.009.VL, CPA.7.009.HC, CPA.7.009.LC, CPA.7.009.H1, CPA.7.009.H2, CPA.7.009.H3 CPA.7.009.H4; CPA.7.009.vhCDR1, CPA.7.009.vhCDR2, CPA.7.009.vhCDR3, CPA.7.009.vlCDR1, CPA.7.009.vlCDR2, and CPA.7.009.vlCDR3;

CPA.7.010, CPA.7.010.VH, CPA.7.010.VL, CPA.7.010.HC, CPA.7.010.LC, CPA.7.010.H1, CPA.7.010.H2, CPA.7.010.H3 CPA.7.010.H4; CPA.7.010.vhCDR1, CPA.7.010.vhCDR2, CPA.7.010.vhCDR3, CPA.7.010.vlCDR1, CPA.7.010.vlCDR2, and CPA.7.010.vlCDR3;

CPA.7.011, CPA.7.011.VH, CPA.7.011.VL, CPA.7.011.HC, CPA.7.011.LC, CPA.7.011.H1, CPA.7.011.H2, CPA.7.011.H3 CPA.7.011.H4; CPA.7.011.vhCDR1, CPA.7.011.vhCDR2, CPA.7.011.vhCDR3, CPA.7.011.vlCDR1, CPA.7.011.vlCDR2, and CPA.7.011.vlCDR3;

CPA.7.012, CPA.7.012.VH, CPA.7.012.VL, CPA.7.012.HC, CPA.7.012.LC, CPA.7.012.H1, CPA.7.012.H2, CPA.7.012.H3 CPA.7.012.H4; CPA.7.012.vhCDR1, CPA.7.012.vhCDR2, CPA.7.012.vhCDR3, CPA.7.012.vlCDR1, CPA.7.012.vlCDR2, and CPA.7.012.vlCDR3;

CPA.7.013, CPA.7.013.VH, CPA.7.013.VL, CPA.7.013.HC, CPA.7.013.LC, CPA.7.013.H1, CPA.7.013.H2, CPA.7.013.H3 CPA.7.013.H4; CPA.7.013.vhCDR1, CPA.7.013.vhCDR2, CPA.7.013.vhCDR3, CPA.7.013.vlCDR1, CPA.7.013.vlCDR2, and CPA.7.013.vlCDR3;

CPA.7.014, CPA.7.014.VH, CPA.7.014.VL, CPA.7.014.HC, CPA.7.014.LC, CPA.7.014.H1, CPA.7.014.H2, CPA.7.014.H3 CPA.7.014.H4; CPA.7.014.vhCDR1, CPA.7.014.vhCDR2, CPA.7.014.vhCDR3, CPA.7.014.vlCDR1, CPA.7.014.vlCDR2, and CPA.7.014.vlCDR3;

CPA.7.015, CPA.7.015.VH, CPA.7.015.VL, CPA.7.015.HC, CPA.7.015.LC, CPA.7.015.H1, CPA.7.015.H2, CPA.7.015.H3 CPA.7.015.H4; CPA.7.015.vhCDR1, CPA.7.015.vhCDR2, CPA.7.015.vhCDR3, CPA.7.015.vlCDR1, CPA.7.015.vlCDR2, and CPA.7.015.vlCDR3;

CPA.7.017, CPA.7.017.VH, CPA.7.017.VL, CPA.7.017.HC, CPA.7.017.LC, CPA.7.017H1, CPA.7.017.H2, CPA.7.017.H3 CPA.7.017.H4; CPA.7.017.vhCDR1, CPA.7.000171.vhCDR2, CPA.7.017.vhCDR3, CPA.7.017.vlCDR1, CPA.7.017.vlCDR2, and CPA.7.017.vlCDR3;

CPA.7.018, CPA.7.018.VH, CPA.7.018.VL, CPA.7.018.HC, CPA.7.018.LC, CPA.7.018.H1, CPA.7.018.H2, CPA.7.018.H3 CPA.7.018.H4; CPA.7.017.vhCDR1, CPA.7.017.vhCDR2, CPA.7.017.vhCDR3, CPA.7.017.vlCDR1, CPA.7.017.vlCDR2, and CPA.7.017.vlCDR3;

CPA.7.019, CPA.7.019.VH, CPA.7.019.VL, CPA.7.019.HC, CPA.7.019.LC, CPA.7.019.H1, CPA.7.019.H2, CPA.7.019.H3 CPA.7.019.H4; CPA.7.019.vhCDR1, CPA.7.019.vhCDR2, CPA.7.019.vhCDR3, CPA.7.019.vlCDR1, CPA.7.019.vlCDR2, and CPA.7.019.vlCDR3;

CPA.7.021, CPA.7.021.VH, CPA.7.021.VL, CPA.7.021.HC, CPA.7.021.LC, CPA.7.021.H1, CPA.7.021.H2, CPA.7.021.H3 CPA.7.021.H4; CPA.7.021.vhCDR1, CPA.7.021.vhCDR2, CPA.7.021.vhCDR3, CPA.7.021.vlCDR1, CPA.7.021.vlCDR2, and CPA.7.021.vlCDR3;

CPA.7.022, CPA.7.022.VH, CPA.7.022.VL, CPA.7.022.HC, CPA.7.022.LC, CPA.7.022.H1, CPA.7.022.H2, CPA.7.022.H3 CPA.7.022.H4; CPA.7.022.vhCDR1, CPA.7.022.vhCDR2, CPA. 7.002201.vhCDR3, CPA.7.022.vlCDR1, CPA.7.022.vlCDR2, and CPA.7.022.vlCDR3;

CPA.7.023, CPA.7.023.VH, CPA.7.023.VL, CPA.7.023.HC, CPA.7.023.LC, CPA.7.023.H1, CPA.7.023.H2, CPA.7.023.H3 CPA.7.023.H4; CPA.7.023.vhCDR1, CPA.7.023.vhCDR2, CPA.7.023.vhCDR3, CPA.7.023.vlCDR1, CPA.7.023.vlCDR2, and CPA.7.023.vlCDR3;

CPA.7.024, CPA.7.024.VH, CPA.7.024.VL, CPA.7.024.HC, CPA.7.024.LC, CPA.7.024.H1, CPA.7.024.H2, CPA.7.024.H3 CPA.7.024.H4; CPA.7.024.vhCDR1, CPA.7.024.vhCDR2, CPA.7.024.vhCDR3, CPA.7.024.vlCDR1, CPA.7.024.vlCDR2, and CPA.7.024.vlCDR3;

CPA.7.033, CPA.7.033.VH, CPA.7.033.VL, CPA.7.033.HC, CPA.7.033.LC, CPA.7.033.H1, CPA.7.033.H2, CPA.7.033.H3 CPA.7.033.H4; CPA.7.033.vhCDR1, CPA.7.033.vhCDR2, CPA.7.033.vhCDR3, CPA.7.033.vlCDR1, CPA.7.033.vlCDR2, and CPA.7.033.vlCDR3;

CPA.7.034, CPA.7.034.VH, CPA.7.034.VL, CPA.7.034.HC, CPA.7.034.LC, CPA.7.034.H1, CPA.7.034.H2, CPA.7.034.H3 CPA.7.034.H4; CPA.7.034.vhCDR1, CPA.7.034.vhCDR2, CPA.7.034.vhCDR3, CPA.7.034.vlCDR1, CPA.7.034.vlCDR2, and CPA.7.034.vlCDR3;

CPA.7.036, CPA.7.036.VH, CPA.7.036.VL, CPA.7.036.HC, CPA.7.036.LC, CPA.7.036.H1, CPA.7.036.H2, CPA.7.036.H3 CPA.7.036.H4; CPA.7.036.vhCDR1, CPA.7.036.vhCDR2, CPA.7.036.vhCDR3, CPA.7.036.vlCDR1, CPA.7.036.vlCDR2, and CPA.7.036.vlCDR3;

CPA.7.040, CPA.7.040.VH, CPA.7.040.VL, CPA.7.040.HC, CPA.7.040.LC, CPA.7.040.H1, CPA.7.040.H2, CPA.7.040.H3 and CPA.7.040.H4; CPA.7.040.vhCDR1, CPA.7.040.vhCDR2, CPA.7.040.vhCDR3, CPA.7.040.vlCDR1, CPA.7.040.vlCDR2, and CPA.7.040.vlCDR3;

CPA.7.046, CPA.7.046.VH, CPA.7.046.VL, CPA.7.046.HC, CPA.7.046.LC, CPA.7.046.H1, CPA.7.046.H2, CPA.7.046.H3 CPA.7.046.H4; CPA.7.046.vhCDR1, CPA.7.046.vhCDR2, CPA.7.046.vhCDR3, CPA.7.046.vlCDR1, CPA.7.046.vlCDR2, and CPA.7.046.vlCDR3;

CPA.7.047, CPA.7.047.VH, CPA.7.047.VL, CPA.7.047.HC, CPA.7.047.LC, CPA.7.047.H1, CPA.7.047.H2, CPA.7.047.H3 CPA.7.047.H4; CPA.7.047.vhCDR1, CPA.7.047.vhCDR2, CPA.7.047.vhCDR3, CPA.7.047.vlCDR1, CPA. 7.004701.vlCDR2, and CPA.7.047.vlCDR3;

CPA.7.049, CPA.7.049.VH, CPA.7.049.VL, CPA.7.049.HC, CPA.7.049.LC, CPA.7.049.H1, CPA.7.049.H2, CPA.7.049.H3 CPA.7.049.H4; CPA.7.049.vhCDR1, CPA.7.049.vhCDR2, CPA.7.049.vhCDR3, CPA.7.049.vlCDR1, CPA.7.049.vlCDR2, and CPA.7.049.vlCDR3; and

CPA.7.050, CPA.7.050.VH, CPA.7.050.VL, CPA.7.050.HC, CPA.7.050.LC, CPA.7.050.H1, CPA.7.050.H2, CPA.7.050.H3 CPA.7.050.H4, CPA.7.050.vhCDR1, CPA.7.050.vhCDR2, CPA.7.050.vhCDR3, CPA.7.050.vlCDR1, CPA.7.050.vlCDR2, and CPA.7.050.vlCDR3.

In addition, there are a number of CPA antibodies generated herein that bound to PVRIG but did not block the interaction of PVRIG and PVLR2. The anti-PVRIG/anti-TIGIT bispecific antibodies of the invention can comprise a PVRIG antibody and/or antigen binding domain sequence capable of binding but not blocking the receptor-ligand interaction as the PVRIG binding portion of the anti-PVRIG/anti-TIGIT bispecific antibodies. The anti-PVRIG/anti-TIGIT bispecific antibodies of the invention can comprise the CDRs from a PVRIG antibody sequence capable of sequence capable of binding but not blocking the receptor-ligand interaction as the PVRIG binding portion of the anti-PVRIG/anti-TIGIT bispecific antibodies. The CPA antibodies, as well as the CDR sequences, that bind but do not block the receptor-ligand interaction are as below, with their components outlined as well, the sequences for which are shown in FIG. 4A-4AA:

CPA.7.028, CPA.7.028.VH, CPA.7.028.VL, CPA.7.028.HC, CPA.7.028.LC, CPA.7.028.H1, CPA.7.028.H2, CPA.7.028.H3 and CPA.7.028.H4; CPA.7.028.vhCDR1, CPA.7.028.vhCDR2, CPA.7.028.vhCDR3, CPA.7.028.vlCDR1, CPA.7.028.vlCDR2, and CPA.7.028.vlCDR3.

CPA.7.030, CPA.7.030.VH, CPA.7.030.VL, CPA.7.030.HC, CPA.7.030.LC, CPA.7.030.H1, CPA.7.030.H2, CPA.7.030.H3 and CPA.7.030.H4; CPA.7.030.vhCDR1, CPA.7.030.vhCDR2, CPA.7.030.vhCDR3, CPA.7.030.vlCDR1, CPA.7.030.vlCDR2, and CPA.7.030.vlCDR3.

CPA.7.041, CPA.7.041.VH, CPA.7.041.VL, CPA.7.041.HC, CPA.7.041.LC, CPA.7.041.H1, CPA.7.041.H2, CPA.7.041.H3 and CPA.7.041.H4; CPA.7.041.vhCDR1, CPA.7.041.vhCDR2, CPA.7.041.vhCDR3, CPA.7.041.vlCDR1, CPA.7.041.vlCDR2, and CPA.7.041.vlCDR3.

CPA.7.016, CPA.7.016.VH, CPA.7.016.VL, CPA.7.016.HC, CPA.7.016.LC, CPA.7.016.H1, CPA.7.016.H2, CPA.7.016.H3 and CPA.7.016.H4; CPA.7.016.vhCDR1, CPA.7.016.vhCDR2, CPA.7.016.vhCDR3, CPA.7.016.vlCDR1, CPA.7.016.vlCDR2, and CPA.7.016.vlCDR3.

CPA.7.020, CPA.7.020.VH, CPA.7.020.VL, CPA.7.020.HC, CPA.7.020.LC, CPA.7.020.H1, CPA.7.020.H2, CPA.7.020.H3 and CPA.7.020.H4; CPA.7.020.vhCDR1, CPA.7.020.vhCDR2, CPA.7.020.vhCDR3, CPA.7.020.vlCDR1, CPA.7.020.vlCDR2, and CPA.7.020.vlCDR3.

CPA.7.038, CPA.7.038.VH, CPA.7.038.VL, CPA.7.038.HC, CPA.7.038.LC, CPA.7.038.H1, CPA.7.038.H2, CPA.7.038.H3 and CPA.7.038.H4; CPA.7.038.vhCDR1, CPA.7.038.vhCDR2, CPA.7.038.vhCDR3, CPA.7.038.vlCDR1, CPA.7.038.vlCDR2, and CPA.7.038.vlCDR3.

CPA.7.044, CPA.7.044.VH, CPA.7.044.VL, CPA.7.044.HC, CPA.7.044.LC, CPA.7.044.H1, CPA.7.044.H2, CPA.7.044.H3 and CPA.7.044.H4; CPA.7.044.vhCDR1, CPA.7.044.vhCDR2, CPA.7.044.vhCDR3, CPA.7.044.vlCDR1, CPA.7.044.vlCDR2, and CPA.7.044.vlCDR3.

CPA.7.045, CPA.7.045.VH, CPA.7.045.VL, CPA.7.045.HC, CPA.7.045.LC, CPA.7.045.H1, CPA.7.045.H2, CPA.7.045.H3 and CPA.7.045.H4; CPA.7.045.vhCDR1, CPA.7.045.vhCDR2, CPA.7.045.vhCDR3, CPA.7.045.vlCDR1, CPA.7.045.vlCDR2, and CPA.7.045.vlCDR3.

As discussed herein, the invention further provides variants of the above components, including variants in the CDRs, as outlined above. In addition, variable heavy chains can be 80%, 90%, 95%, 98% or 99% identical to the “VH” sequences herein, and/or contain from 1, 2, 3, 4, 5, 6, 7, 8, 9, 10 amino acid changes, or more, when Fc variants are used. Variable light chains are provided that can be 80%, 90%, 95%, 98% or 99% identical to the “VL” sequences herein, and/or contain from 1, 2, 3, 4, 5, 6, 7, 8, 9, 10 amino acid changes, or more, when Fc variants are used. Similarly, heavy and light chains are provided that are 80%, 90%, 95%, 98% or 99% identical to the “HC” and “LC” sequences herein, and/or contain from 1, 2, 3, 4, 5, 6, 7, 8, 9, 10 amino acid changes, or more, when Fc variants are used. The anti-PVRIG/anti-TIGIT bispecific antibodies of the invention can comprise any of these PVRIG antibody and/or antigen bindgin domain sequences as the PVRIG binding portion of the anti-PVRIG/anti-TIGIT bispecific antibodies.

Furthermore, the present invention provides a number of CHA antibodies, which are murine antibodies generated from hybridomas. As is well known the art, the six CDRs are useful when put into either human framework variable heavy and variable light regions or when the variable heavy and light domains are humanized.

The anti-PVRIG and/or anti-PVRIG/anti-TIGIT bispecific antibodies of the invention can comprise any of the following CHA sets of CDRs from PVRIG antibody sequences as part of the PVRIG binding portion of the anti-PVRIG/anti-TIGIT bispecific antibodies of the invention. Accordingly, the present invention provides anti-PVRIG/anti-TIGIT bispecific antibodies, that comprise the following CHA sets of CDRs as part of the PVRIG binding portion of the anti-PVRIG/anti-TIGIT bispecific antibody, the sequences of which are shown in FIG. 7A-7AE:

CHA.7.502.vhCDR1, CHA.7.502.vhCDR2, CHA.7.502.vhCDR3, CHA.7.502.vlCDR1, CHA.7.502.vlCDR2, and CHA.7.502.vlCDR3.

CHA.7.503.vhCDR1, CHA.7.503.vhCDR2, CHA.7.503.vhCDR3, CHA.7.503.vlCDR1, CHA.7.503.vlCDR2, and CHA.7.503.vlCDR3.

CHA.7.506.vhCDR1, CHA.7.506.vhCDR2, CHA.7.506.vhCDR3, CHA.7.506.vlCDR1, CHA.7.506.vlCDR2, and CHA.7.506.vlCDR3.

CHA.7.508.vhCDR1, CHA.7.508.vhCDR2, CHA.7.508.vhCDR3, CHA.7.508.vlCDR1, CHA.7.508.vlCDR2, and CHA.7.508.vlCDR3.

CHA.7.510.vhCDR1, CHA.7.510.vhCDR2, CHA.7.510.vhCDR3, CHA.7.510.vlCDR1, CHA.7.510.vlCDR2, and CHA.7.510.vlCDR3.

CHA.7.512.vhCDR1, CHA.7.512.vhCDR2, CHA.7.512.vhCDR3, CHA.7.512.vlCDR1, CHA.7.512.vlCDR2, and CHA.7.512.vlCDR3.

CHA.7.514.vhCDR1, CHA.7.514.vhCDR2, CHA.7.514.vhCDR3, CHA.7.514.vlCDR1, CHA.7.514.vlCDR2, and CHA.7.514.vlCDR3.

CHA.7.516.vhCDR1, CHA.7.516.vhCDR2, CHA.7.516.vhCDR3, CHA.7.516.vlCDR1, CHA.7.516.vlCDR2, and CHA.7.516.vlCDR3.

CHA.7.518.vhCDR1, CHA.7.518.vhCDR2, CHA.7.518.vhCDR3, CHA.7.518.vlCDR1, CHA.7.518.vlCDR2, and CHA.7.518.vlCDR3.

CHA.7.520_1.vhCDR1, CHA.7.520_1.vhCDR2, CHA.7.520_1.vhCDR3, CHA.7.520_1.vlCDR1, CHA.7.520_1.vlCDR2, and CHA.7.520_1.vlCDR3.

CHA.7.520_2.vhCDR1, CHA.7.520_2.vhCDR2, CHA.7.520_2.vhCDR3, CHA.7.520_2.vlCDR1, CHA.7.520_2.vlCDR2, and CHA.7.520_2.vlCDR3.

CHA.7.522.vhCDR1, CHA.7.522.vhCDR2, CHA.7.522.vhCDR3, CHA.7.522.vlCDR1, CHA.7.522.vlCDR2, and CHA.7.522.vlCDR3.

CHA.7.524.vhCDR1, CHA.7.524.vhCDR2, CHA.7.524.vhCDR3, CHA.7.524.vlCDR1, CHA.7.524.vlCDR2, and CHA.7.524.vlCDR3.

CHA.7.526.vhCDR1, CHA.7.526.vhCDR2, CHA.7.526.vhCDR3, CHA.7.526.vlCDR1, CHA.7.526.vlCDR2, and CHA.7.526.vlCDR3.

CHA.7.527.vhCDR1, CHA.7.527.vhCDR2, CHA.7.527.vhCDR3, CHA.7.527.vlCDR1, CHA.7.527.vlCDR2, and CHA.7.527.vlCDR3.

CHA.7.528.vhCDR1, CHA.7.528.vhCDR2, CHA.7.528.vhCDR3, CHA.7.528.vlCDR1, CHA.7.528.vlCDR2, and CHA.7.528.vlCDR3.

CHA.7.530.vhCDR1, CHA.7.530.vhCDR2, CHA.7.530.vhCDR3, CHA.7.530.vlCDR1, CHA.7.530.vlCDR2, and CHA.7.530.vlCDR3.

CHA.7.534.vhCDR1, CHA.7.534.vhCDR2, CHA.7.534.vhCDR3, CHA.7.534.vlCDR1, CHA.7.534.vlCDR2, and CHA.7.534.vlCDR3.

CHA.7.535.vhCDR1, CHA.7.535.vhCDR2, CHA.7.535.vhCDR3, CHA.7.535.vlCDR1, CHA.7.535.vlCDR2, and CHA.7.535.vlCDR3.

CHA.7.537.vhCDR1, CHA.7.537.vhCDR2, CHA.7.537.vhCDR3, CHA.7.537.vlCDR1, CHA.7.537.vlCDR2, and CHA.7.537.vlCDR3.

CHA.7.538_1.vhCDR1, CHA.7.538_1.vhCDR2, CHA.7.538_1.vhCDR3, CHA.7.538_1.vlCDR1, CHA.7.538_1.vlCDR2, and CHA.7.538_1.vlCDR3.

CHA.7.538_2.vhCDR1, CHA.7.538_2.vhCDR2, CHA.7.538_2.vhCDR3, CHA.7.538_2.vlCDR1, CHA.7.538_2.vlCDR2, and CHA.7.538_2.vlCDR3.

CHA.7.543.vhCDR1, CHA.7.543.vhCDR2, CHA.7.543.vhCDR3, CHA.7.543.vlCDR1, CHA.7.543.vlCDR2, and CHA.7.543.vlCDR3.

CHA.7.544.vhCDR1, CHA.7.544.vhCDR2, CHA.7.544.vhCDR3, CHA.7.544.vlCDR1, CHA.7.544.vlCDR2, and CHA.7.544.vlCDR3.

CHA.7.545.vhCDR1, CHA.7.545.vhCDR2, CHA.7.545.vhCDR3, CHA.7.545.vlCDR1, CHA.7.545.vlCDR2, and CHA.7.545.vlCDR3.

CHA.7.546.vhCDR1, CHA.7.546.vhCDR2, CHA.7.546.vhCDR3, CHA.7.546.vlCDR1, CHA.7.546.vlCDR2, and CHA.7.546.vlCDR3.

CHA.7.547.vhCDR1, CHA.7.547.vhCDR2, CHA.7.547.vhCDR3, CHA.7.547.vlCDR1, CHA.7.547.vlCDR2, and CHA.7.547.vlCDR3.

CHA.7.548.vhCDR1, CHA.7.548.vhCDR2, CHA.7.548.vhCDR3, CHA.7.548.vlCDR1, CHA.7.548.vlCDR2, and CHA.7.548.vlCDR3.

CHA.7.549.vhCDR1, CHA.7.549.vhCDR2, CHA.7.549.vhCDR3, CHA.7.549.vlCDR1, CHA.7.549.vlCDR2, and CHA.7.549.vlCDR3.

CHA.7.550.vhCDR1, CHA.7.550.vhCDR2, CHA.7.550.vhCDR3, CHA.7.550.vlCDR1, CHA.7.550.vlCDR2, and CHA.7.550.vlCDR3.

CHA.7.518.4.vhCDR1, CHA.7.518.4.vhCDR2, CHA.7.518.4.vhCDR3, CHA.7.518.4.vlCDR1, CHA.7.518.4.vlCDR2, and CHA.7.518.4.vlCDR3.

As above, these sets of CDRs may also be amino acid variants as described above.

In addition, the framework regions of the variable heavy and variable light chains can be humanized as is known in the art (with occasional variants generated in the CDRs as needed), and thus humanized variants of the VH and VL chains of FIGS. 7A-7DD can be generated. Furthermore, the humanized variable heavy and light domains can then be fused with human constant regions, such as the constant regions from IgG1, IgG2, IgG3 and IgG4.

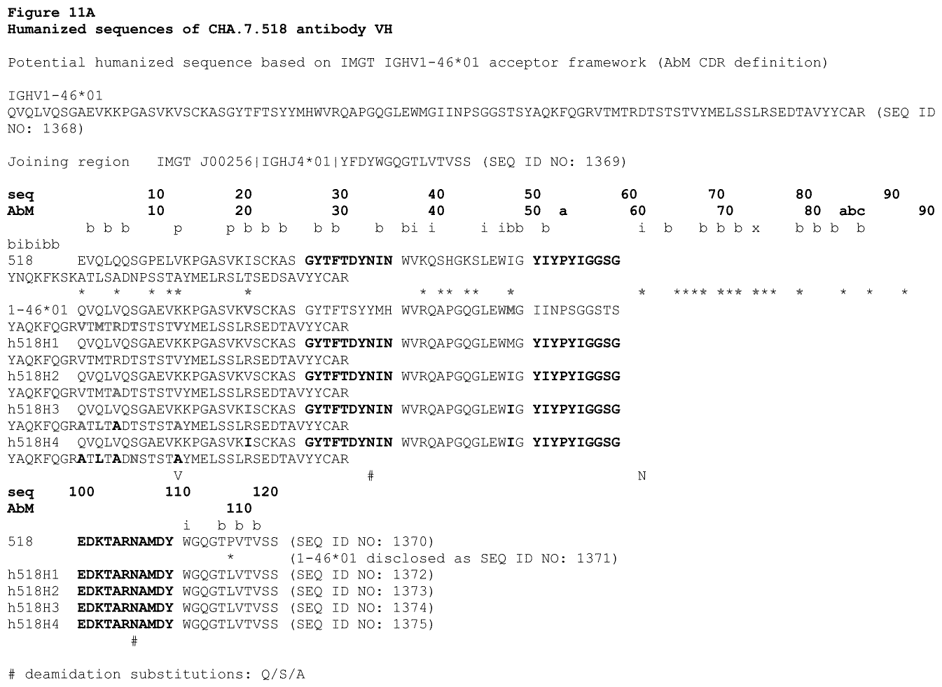

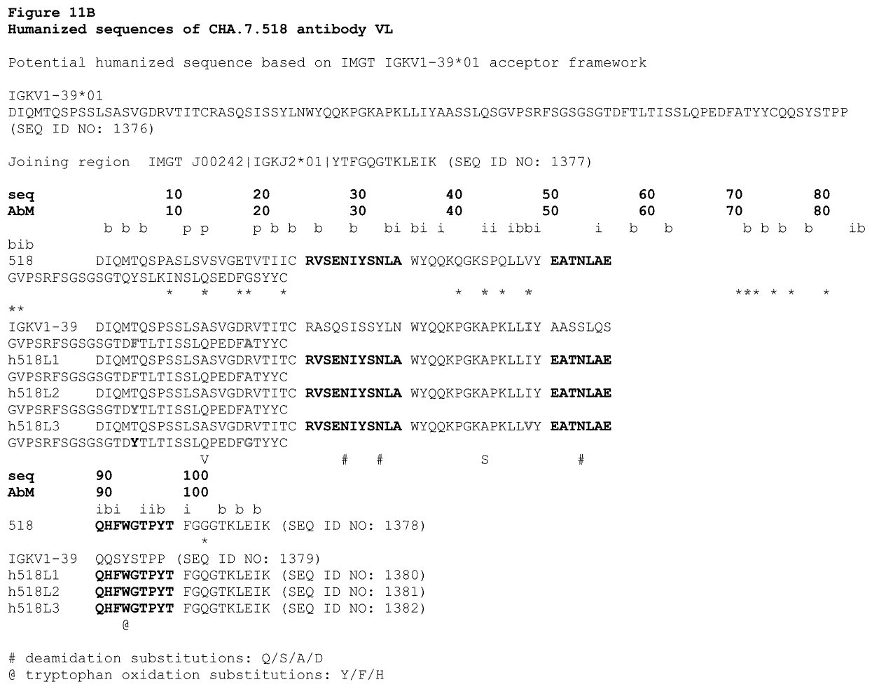

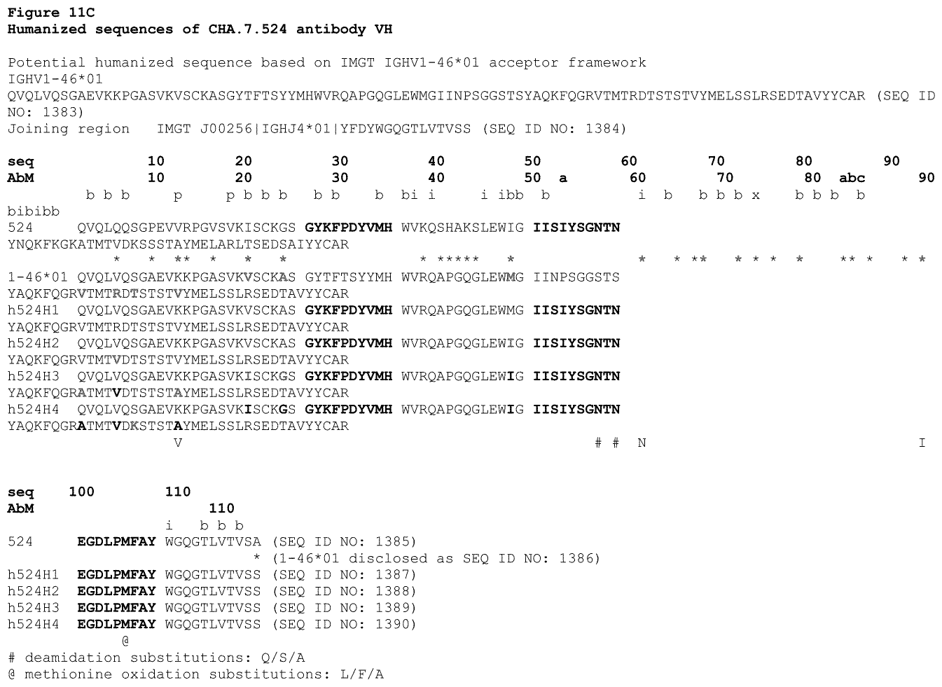

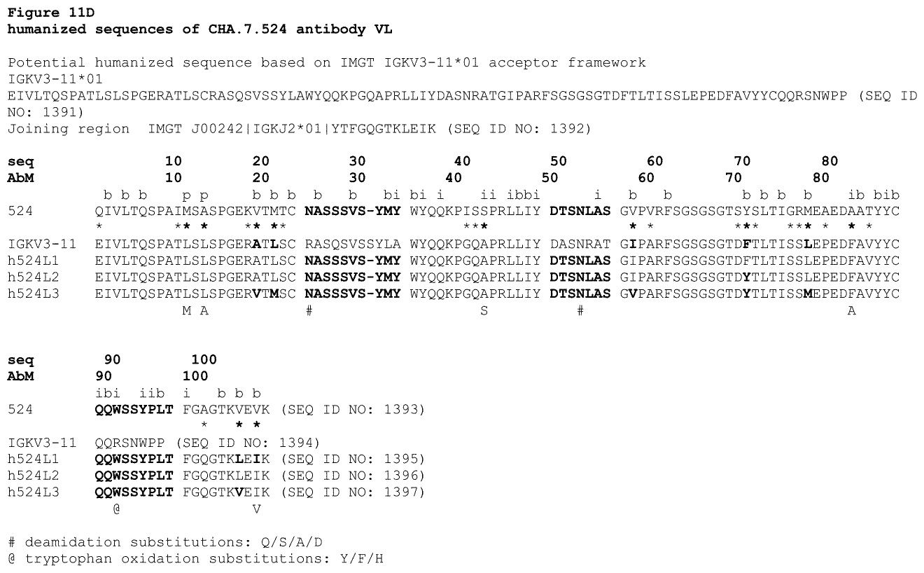

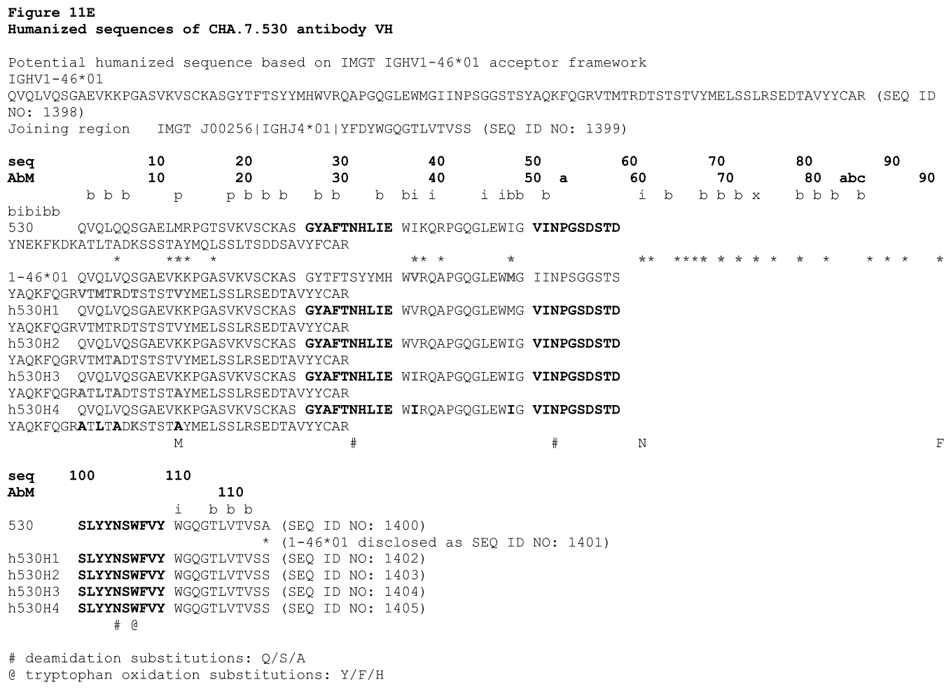

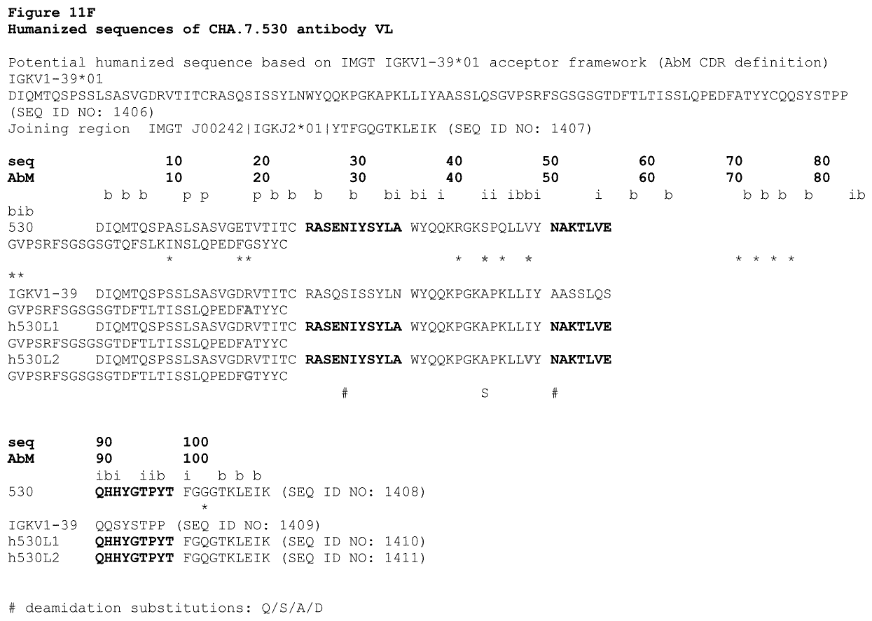

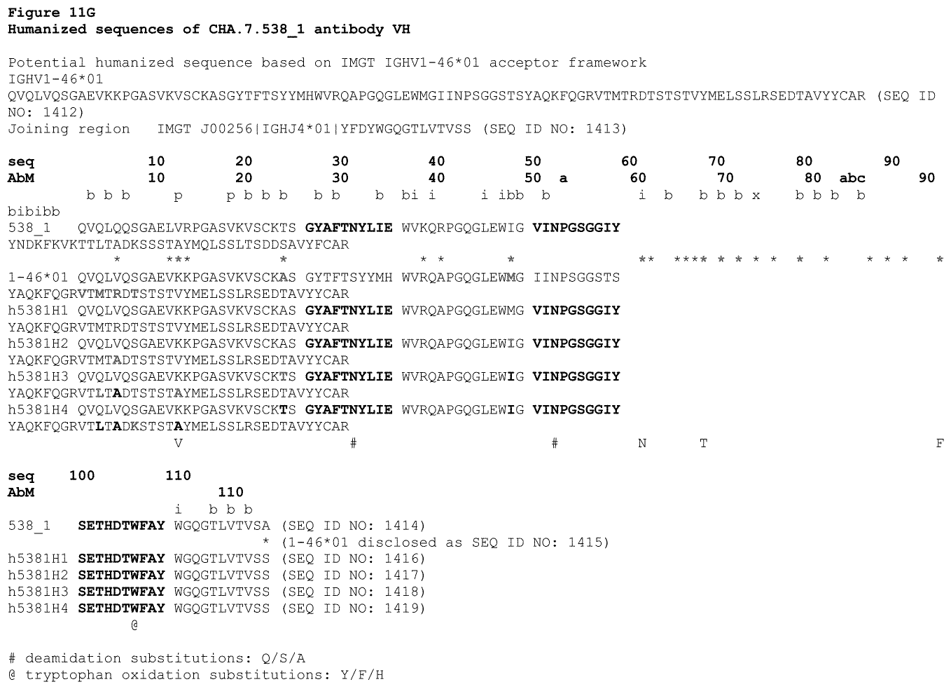

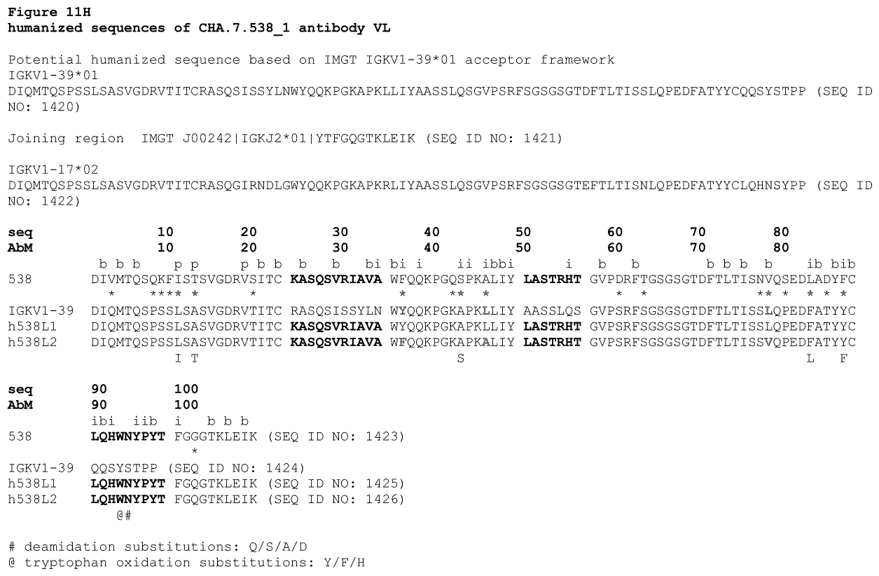

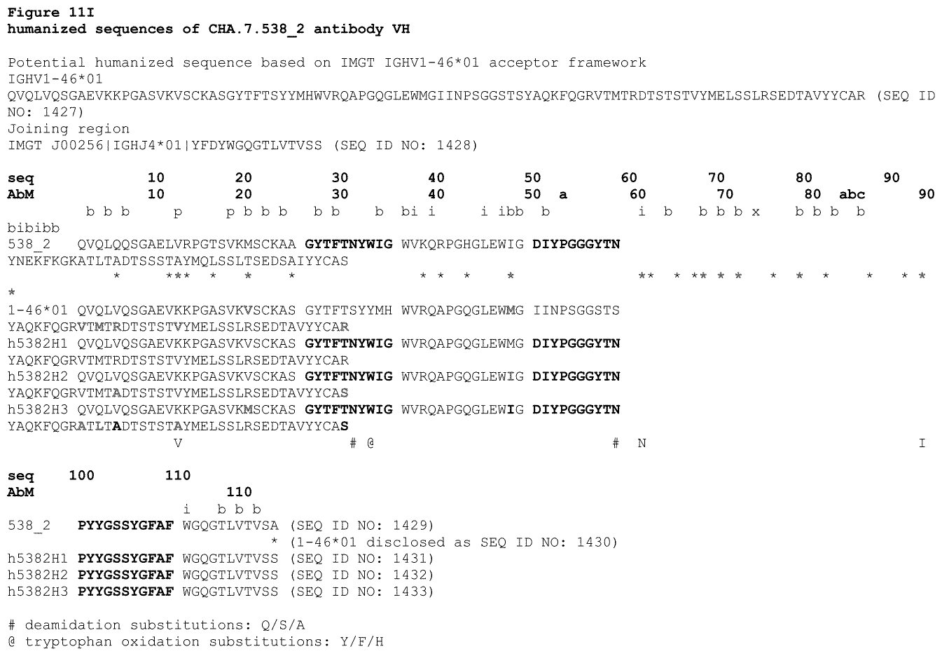

In particular, as is known in the art, murine VH and VL chains can be humanized as is known in the art, for example, using the IgBLAST program of the NCBI website, as outlined in Ye et al. Nucleic Acids Res. 41:W34-W40 (2013), herein incorporated by reference in its entirety for the humanization methods. IgBLAST takes a murine VH and/or VL sequence and compares it to a library of known human germline sequences. As shown herein, for the humanized sequences generated herein, the databases used were IMGT human VH genes (F+ORF, 273 germline sequences) and IMGT human VL kappa genes (F+ORF, 74 germline sequences). An exemplary five CHA sequences were chosen: CHA.7.518, CHA.7.530, CHA.7.538_1, CHA.7.538_2 and CHA.7.524 (see FIGS. 7A-7DD for the VH and VL sequences). For this embodiment of the humanization, human germline IGHV1-46(allele1) was chosen for all 5 as the acceptor sequence and the human heavy chain IGHJ4(allele1) joining region (J gene). For three of four (CHA.7.518, CHA.7.530, CHA.7.538_1 and CHA.7.538_2), human germline IGKV1-39(allele 1) was chosen as the acceptor sequence and human light chain IGKJ2(allele1) (J gene) was chosen. The J gene was chosen from human joining region sequences compiled at IMGT® the international ImMunoGeneTics information system as www.imgt.org. CDRs were defined according to the AbM definition (see www.bioinfo.org.uk/abs/). FIG. 11A-11I depict humanized sequences as well as some potential changes to optimize binding to PVRIG. The anti-PVRIG/anti-TIGIT bispecific antibodies of the invention can comprise any of these humanized PVRIG antibody or antigen binding domain sequences as the PVRIG binding portion of the anti-PVRIG/anti-TIGIT bispecific antibodies. The anti-PVRIG/anti-TIGIT bispecific antibodies of the invention can comprise CHA.7.518 PVRIG antibody sequences as the PVRIG binding portion of the anti-PVRIG/anti-TIGIT bispecific antibodies. The anti-PVRIG/anti-TIGIT bispecific antibodies of the invention can comprise CHA.7.530 PVRIG antibody sequences as the PVRIG binding portion of the anti-PVRIG/anti-TIGIT bispecific antibodies. The anti-PVRIG/anti-TIGIT bispecific antibodies of the invention can comprise CHA.7.538_1 PVRIG antibody sequences as the PVRIG binding portion of the anti-PVRIG/anti-TIGIT bispecific antibodies. The anti-PVRIG/anti-TIGIT bispecific antibodies of the invention can comprise CHA.7.538_2 PVRIG antibody sequences as the PVRIG binding portion of the anti-PVRIG/anti-TIGIT bispecific antibodies. The anti-PVRIG/anti-TIGIT bispecific antibodies of the invention can comprise CHA.7.518.4 PVRIG antibody sequences as the PVRIG binding portion of the anti-PVRIG/anti-TIGIT bispecific antibodies

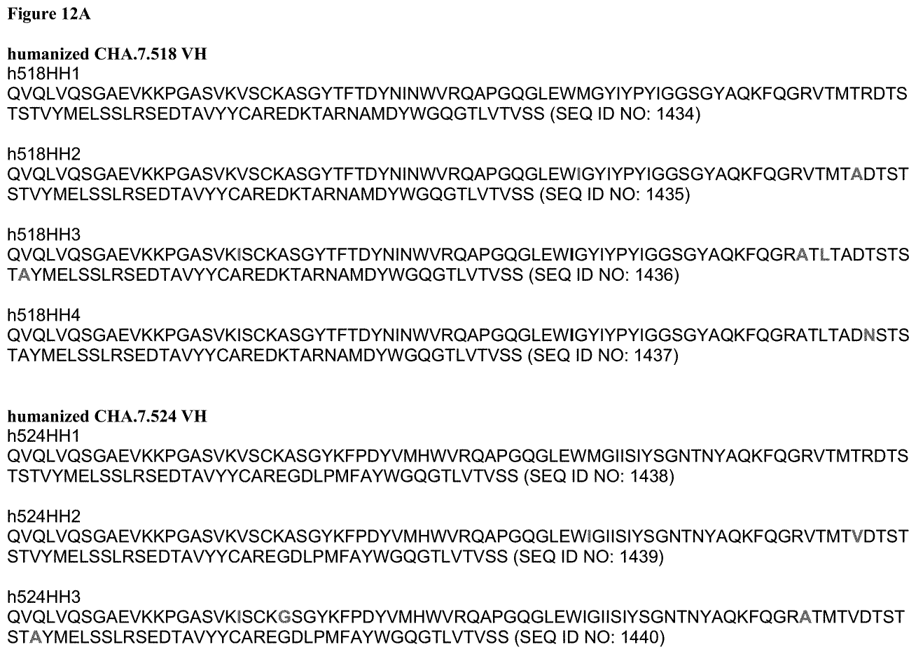

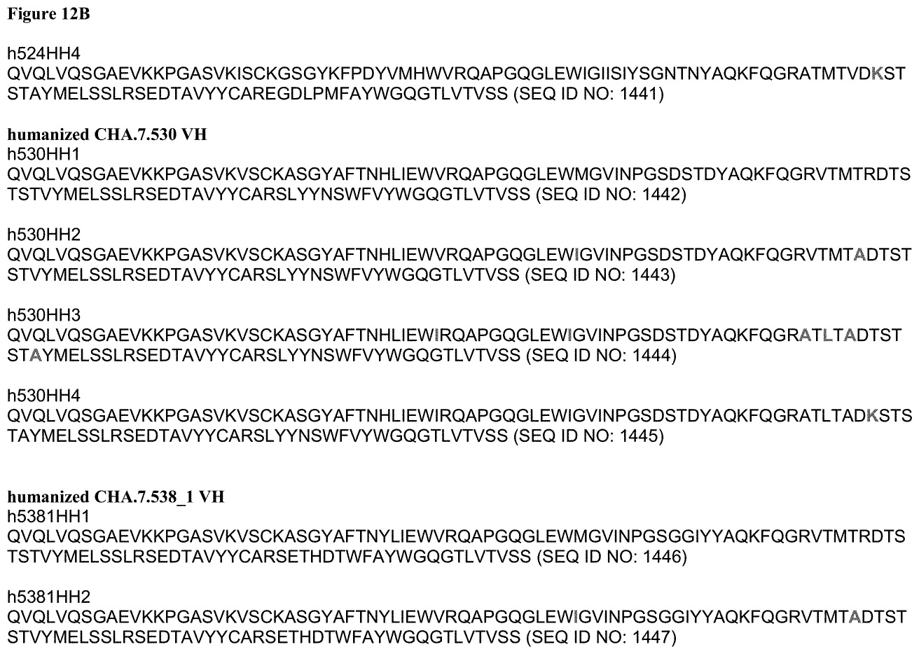

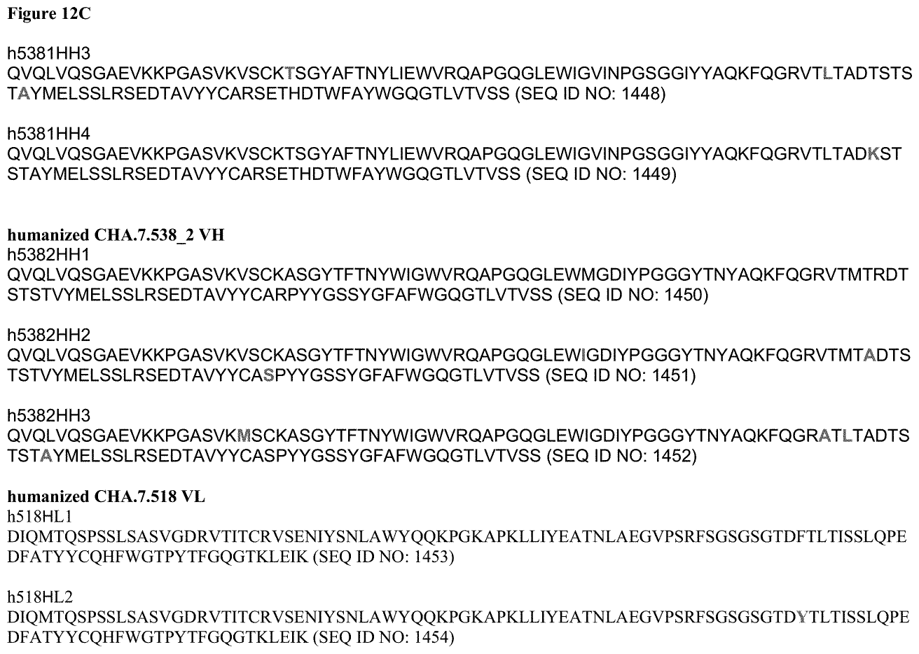

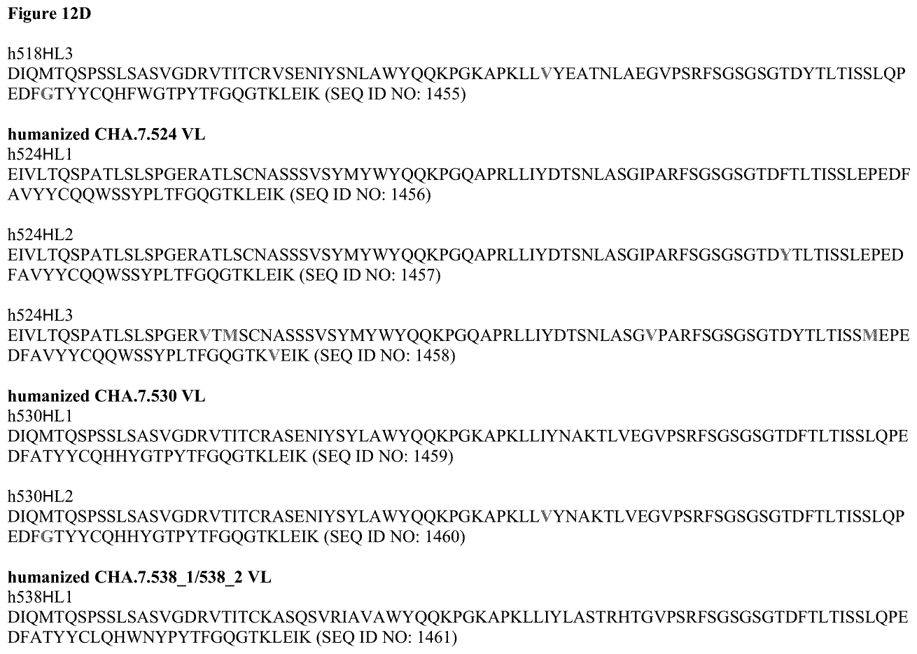

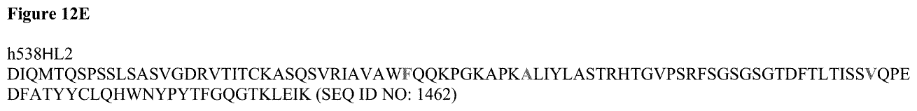

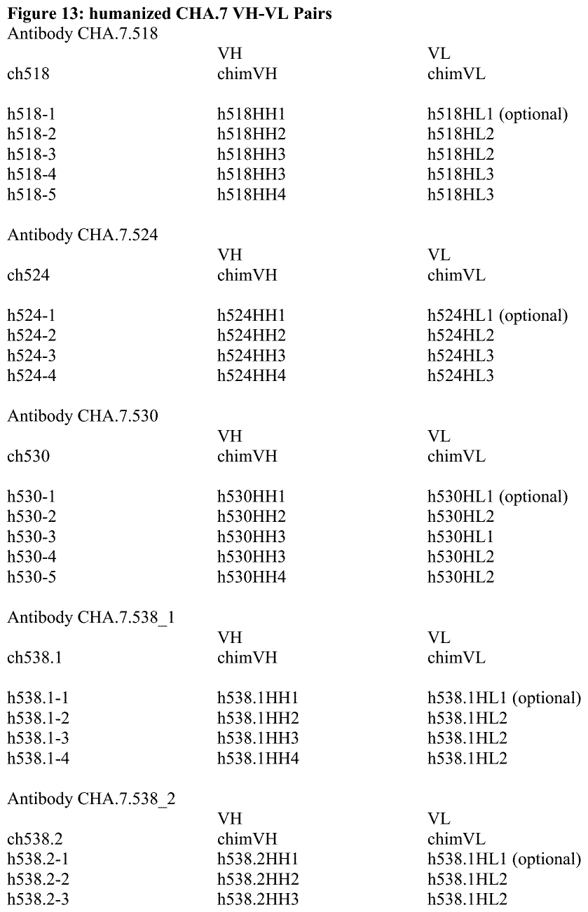

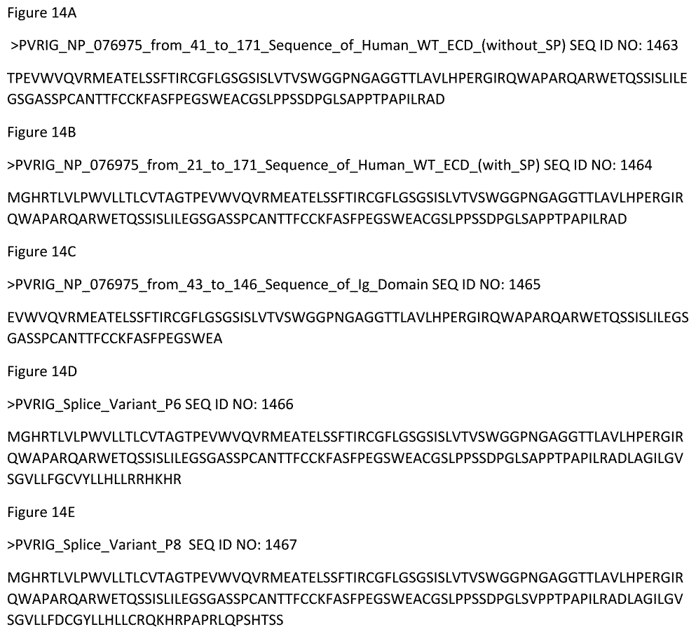

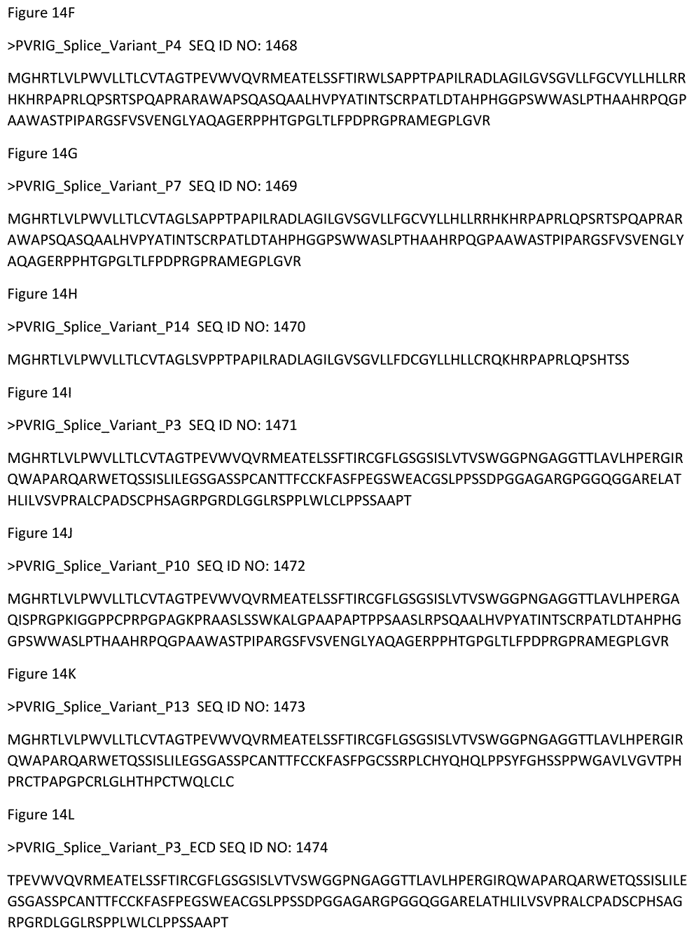

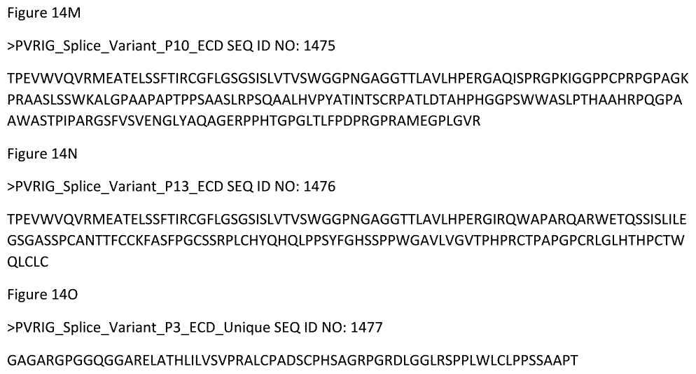

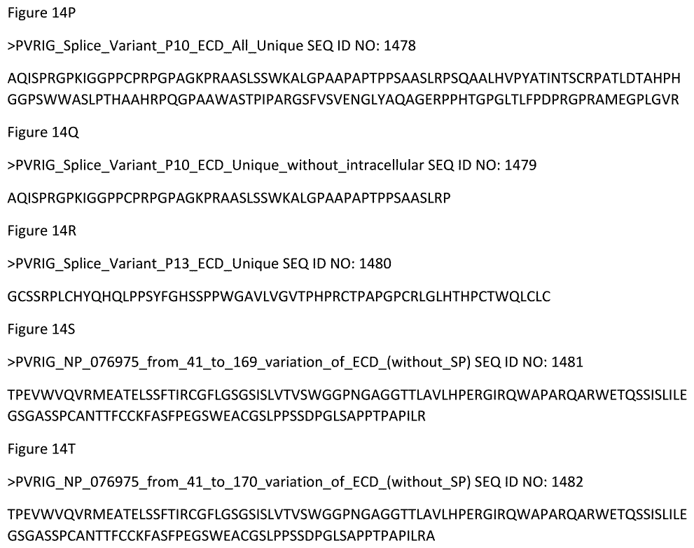

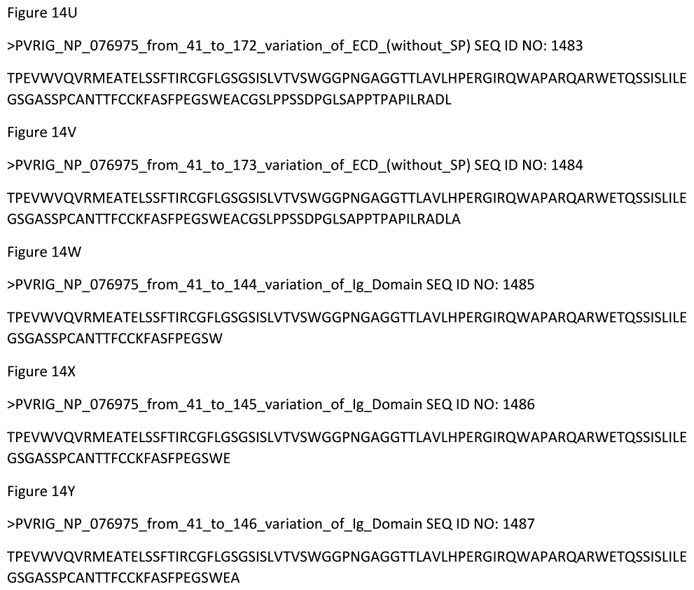

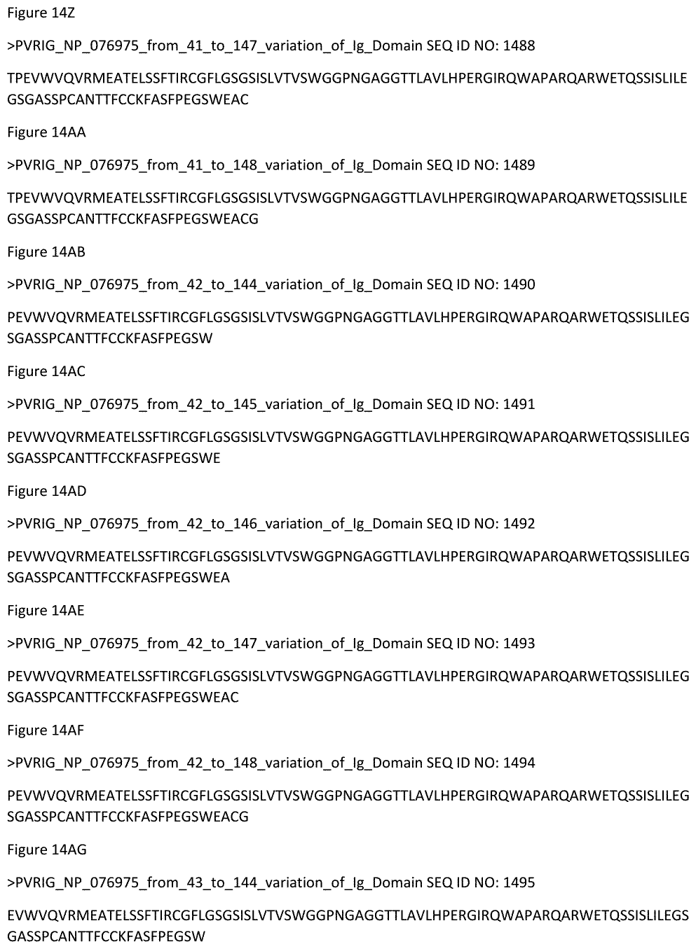

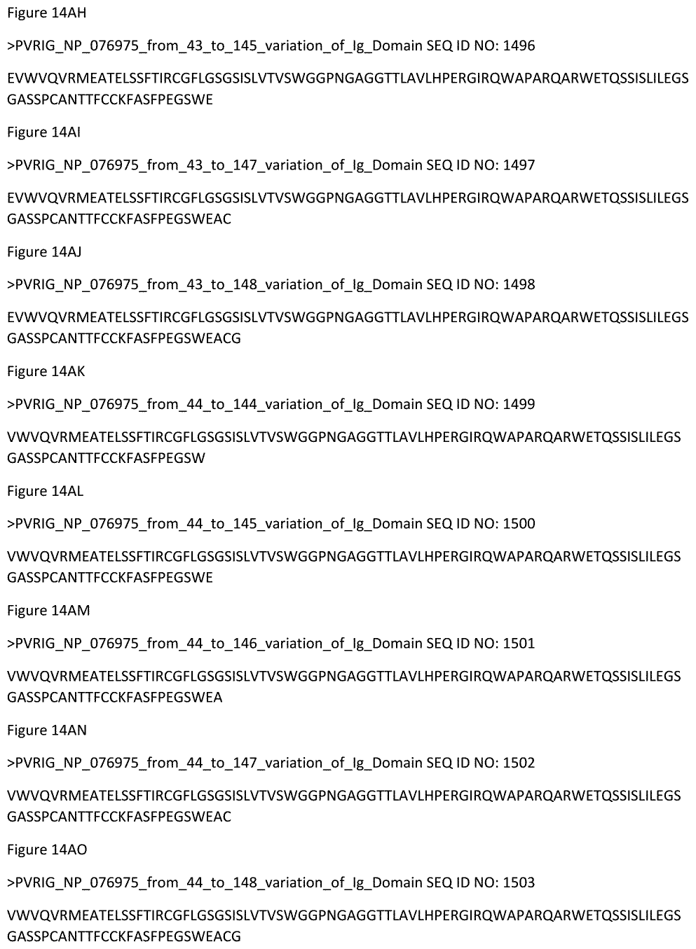

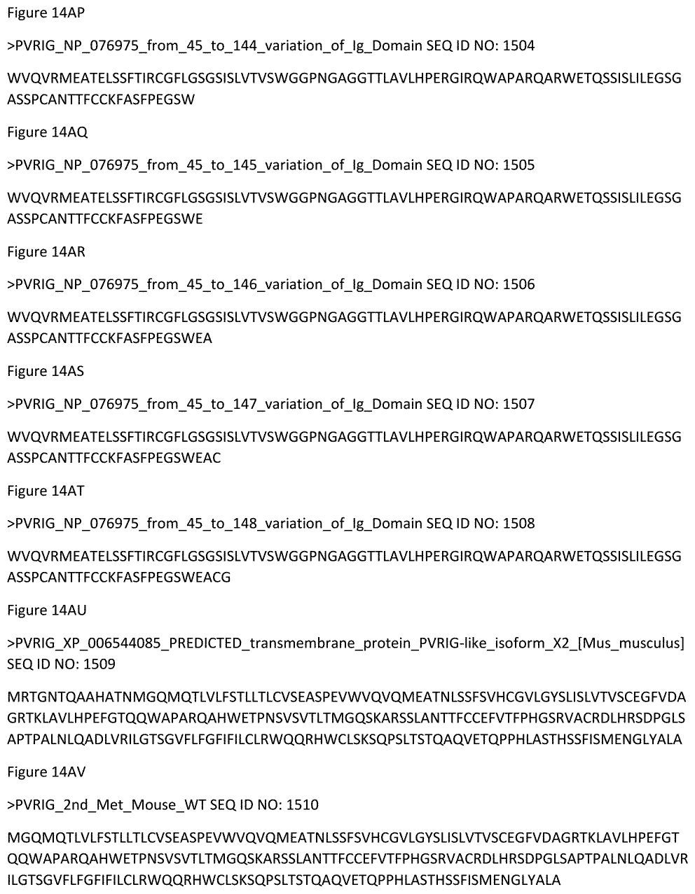

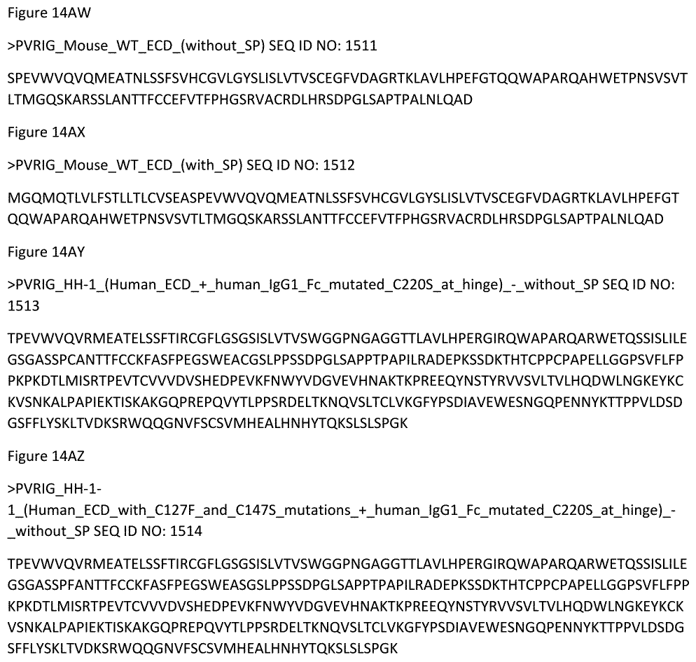

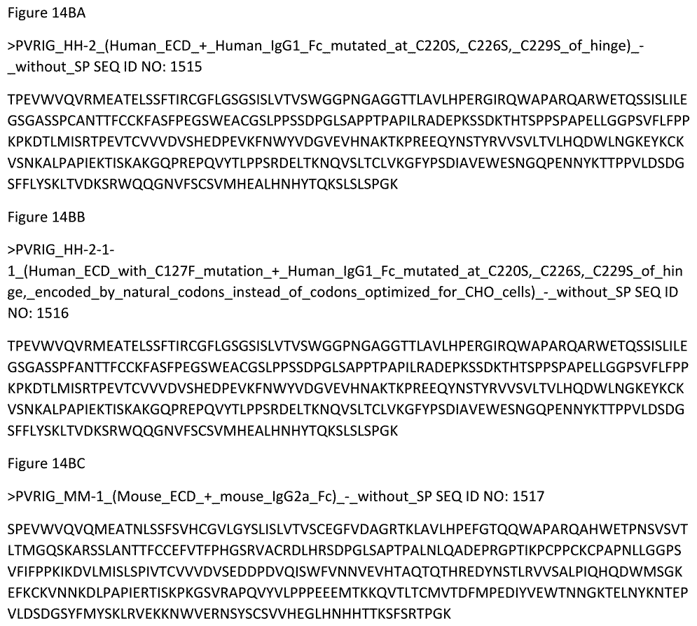

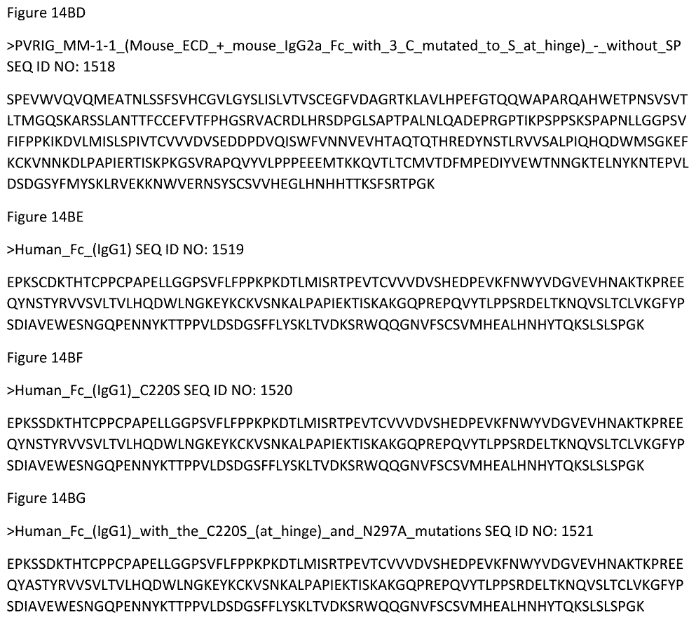

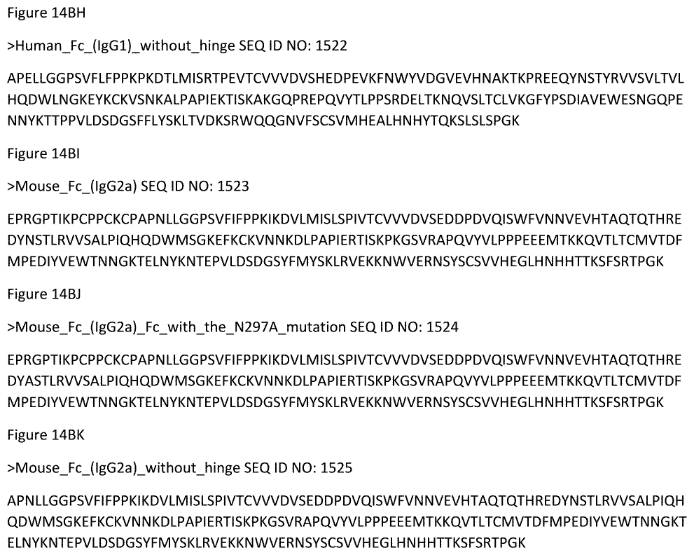

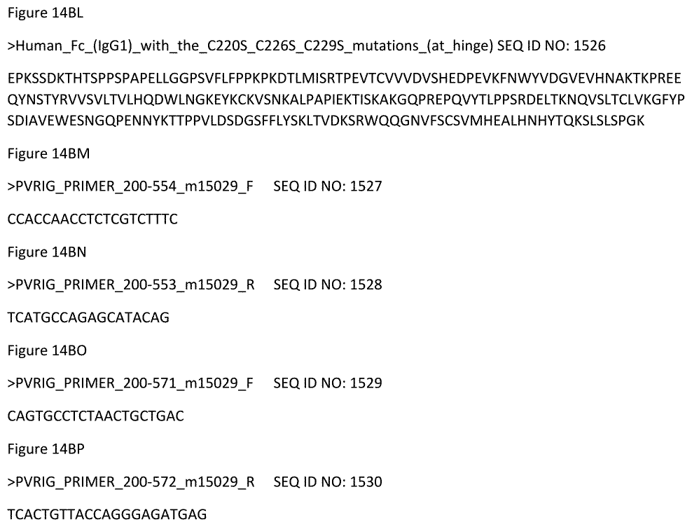

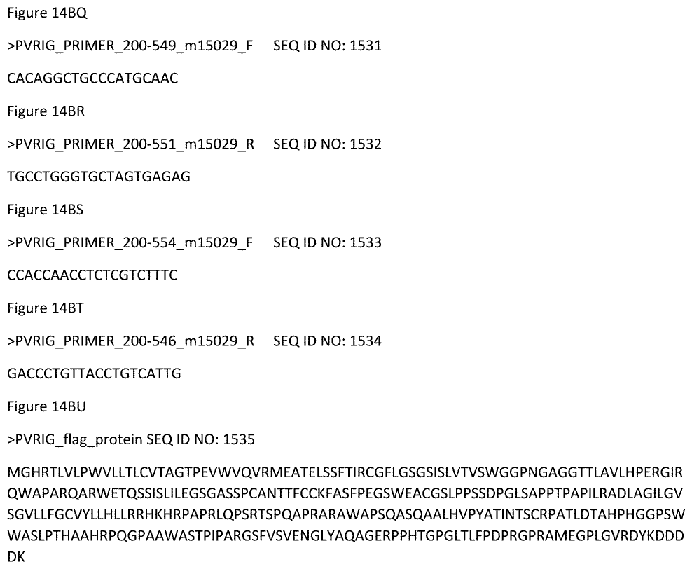

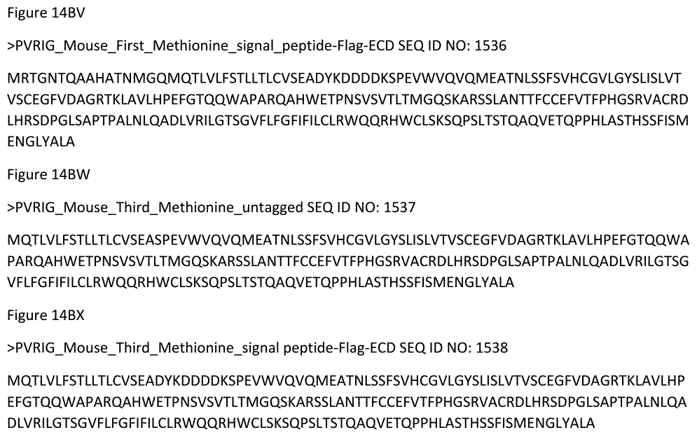

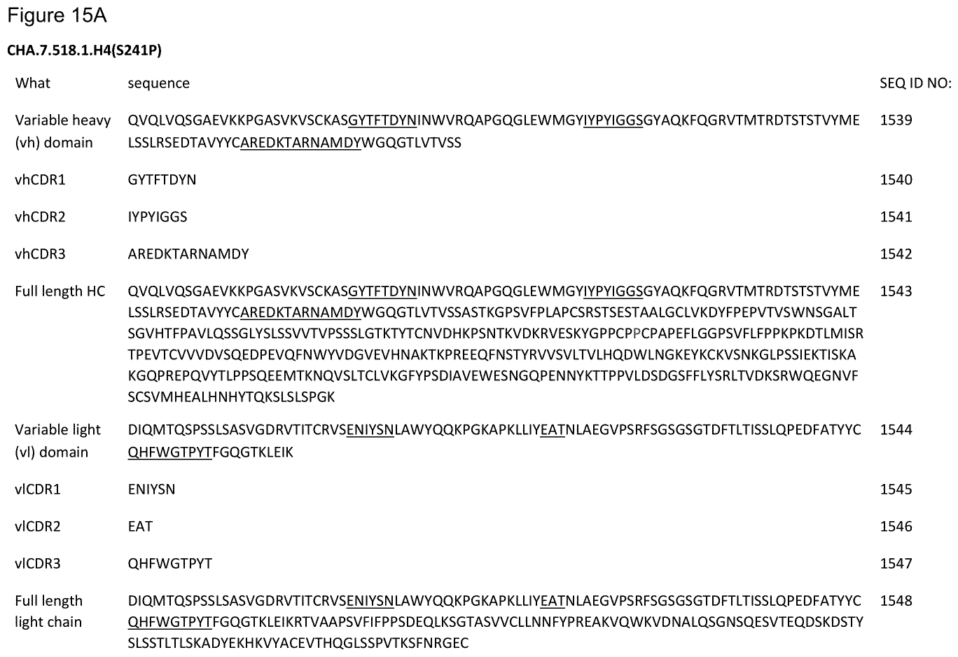

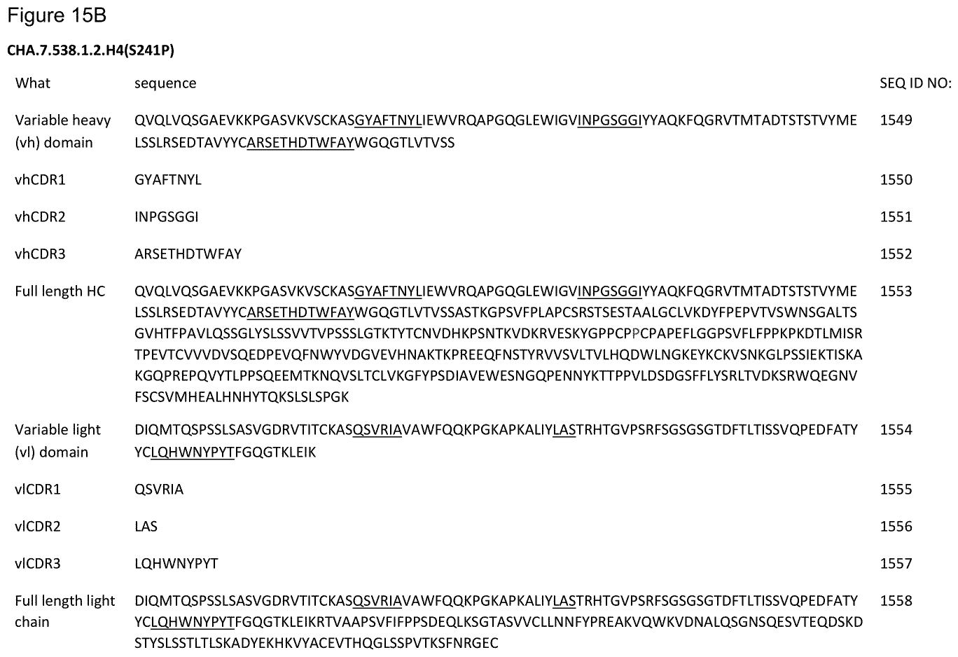

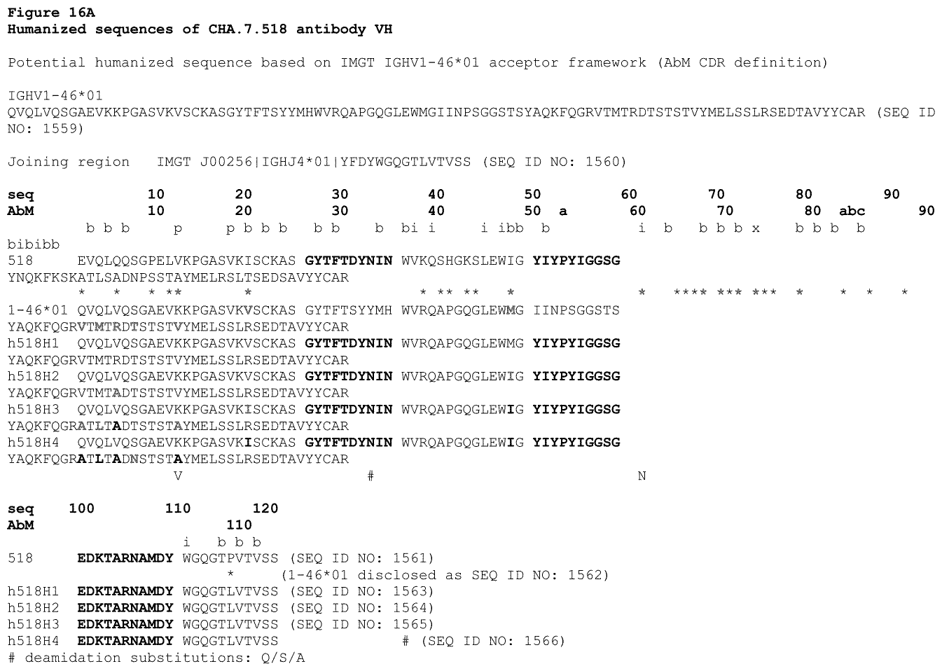

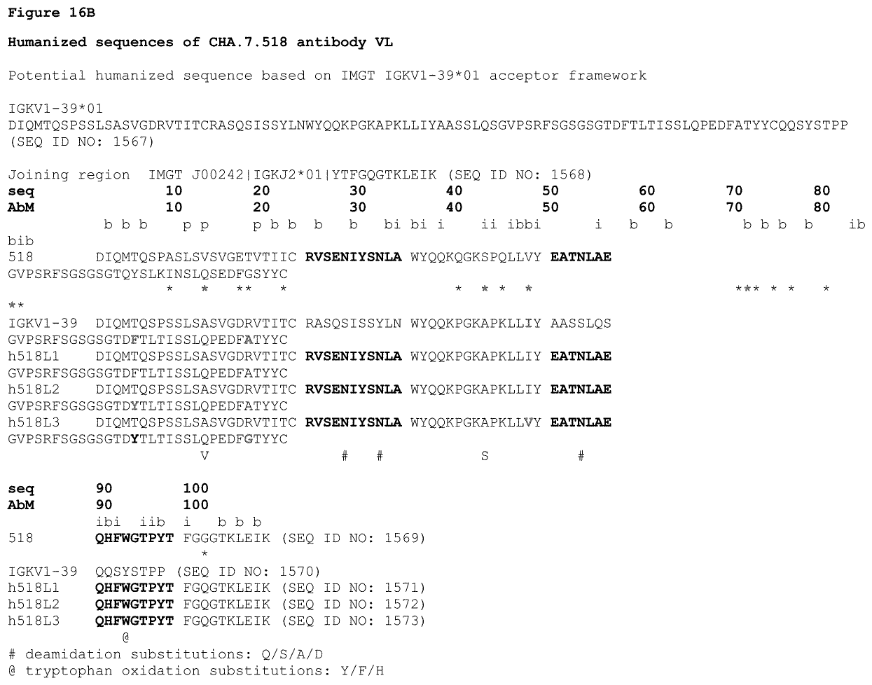

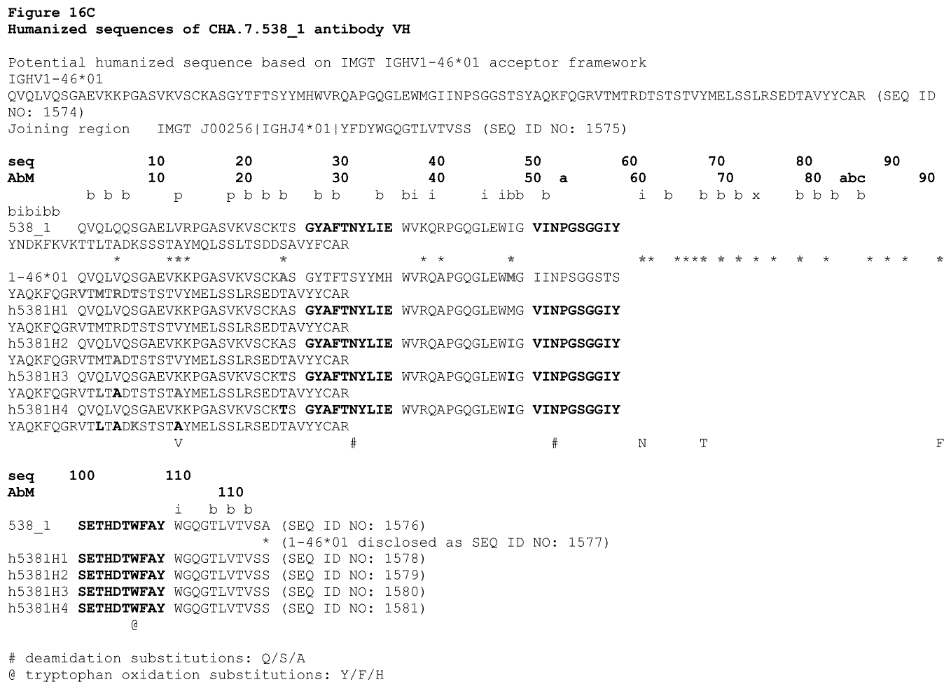

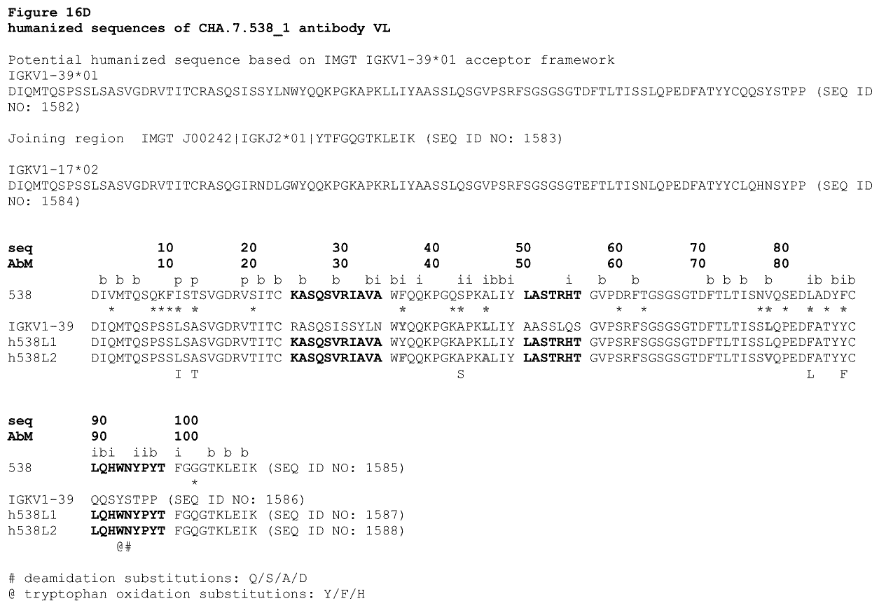

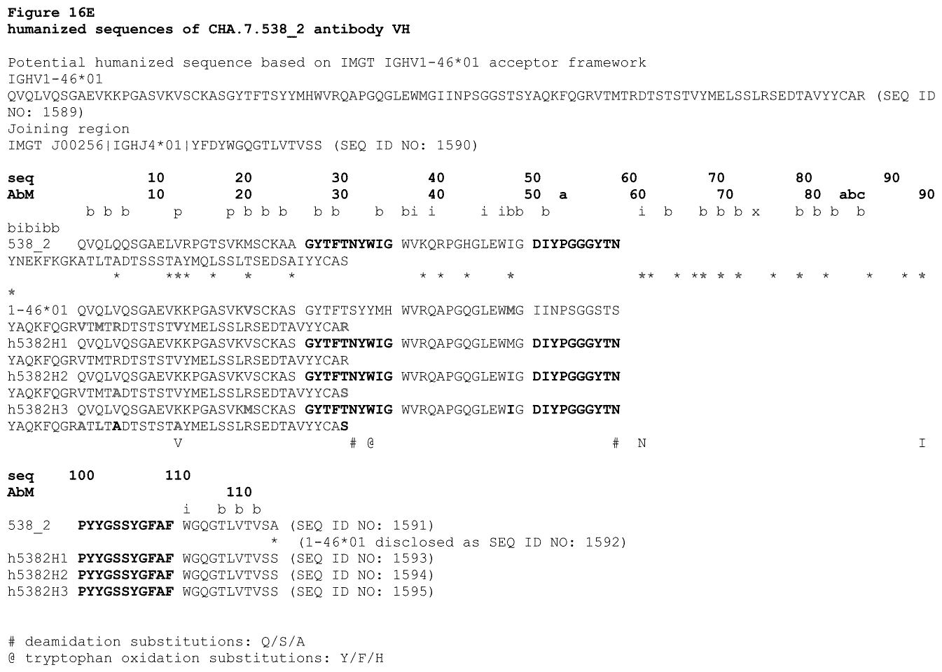

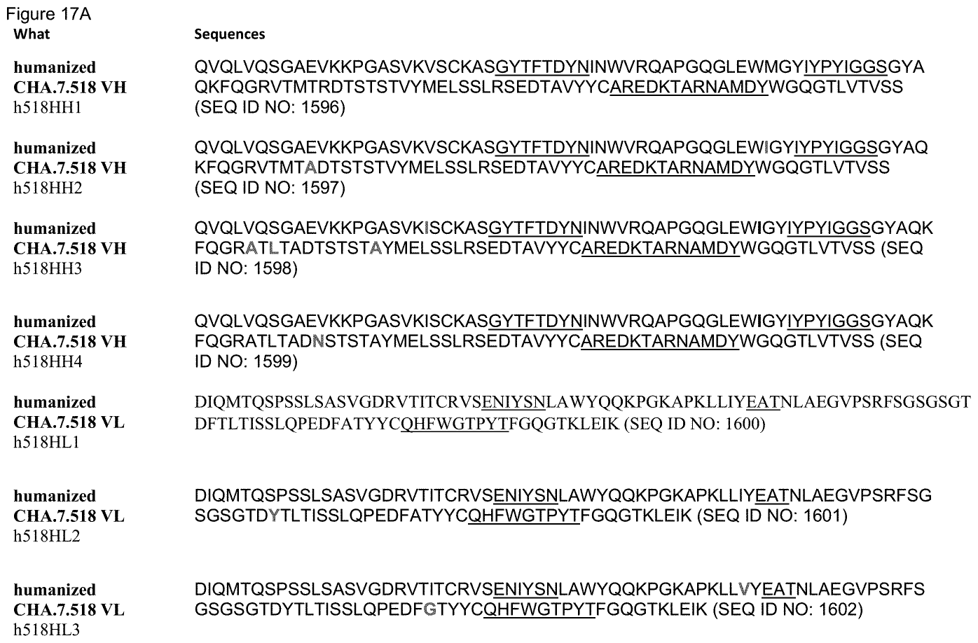

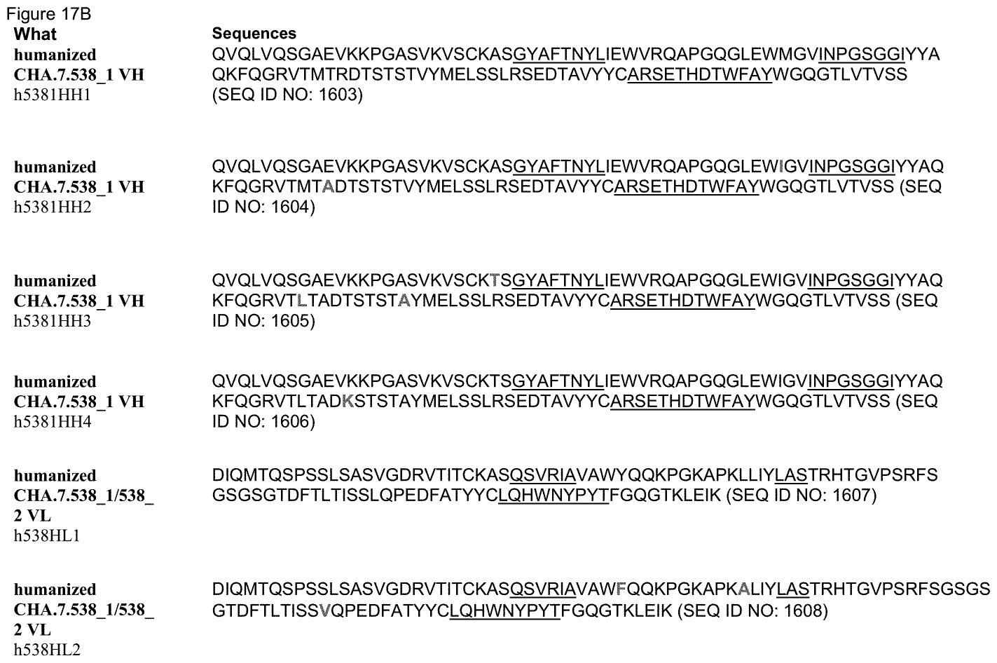

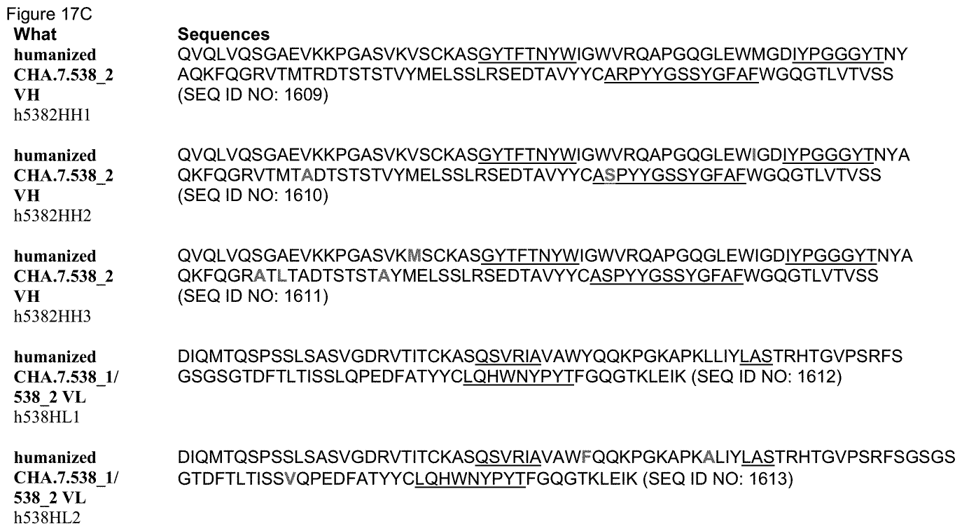

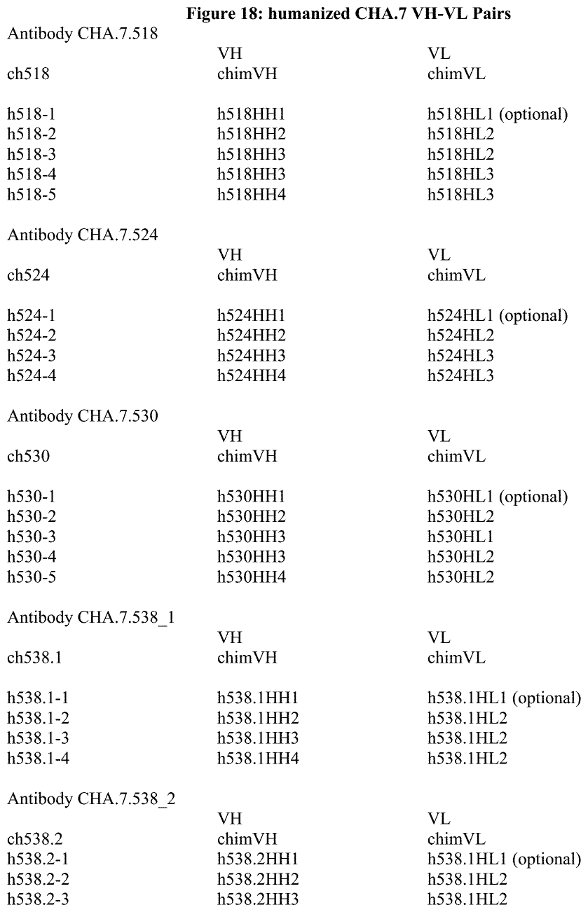

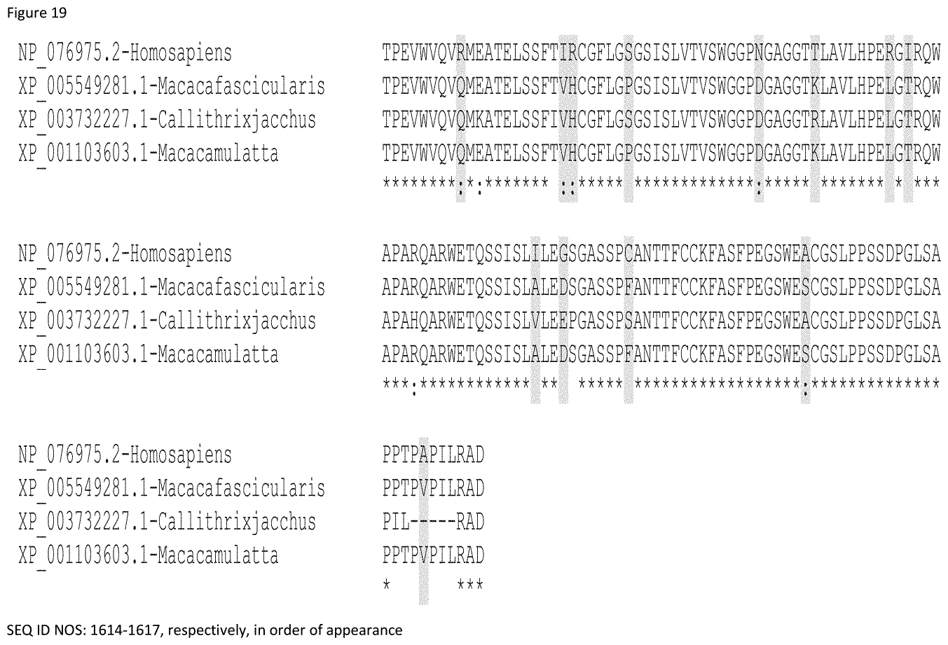

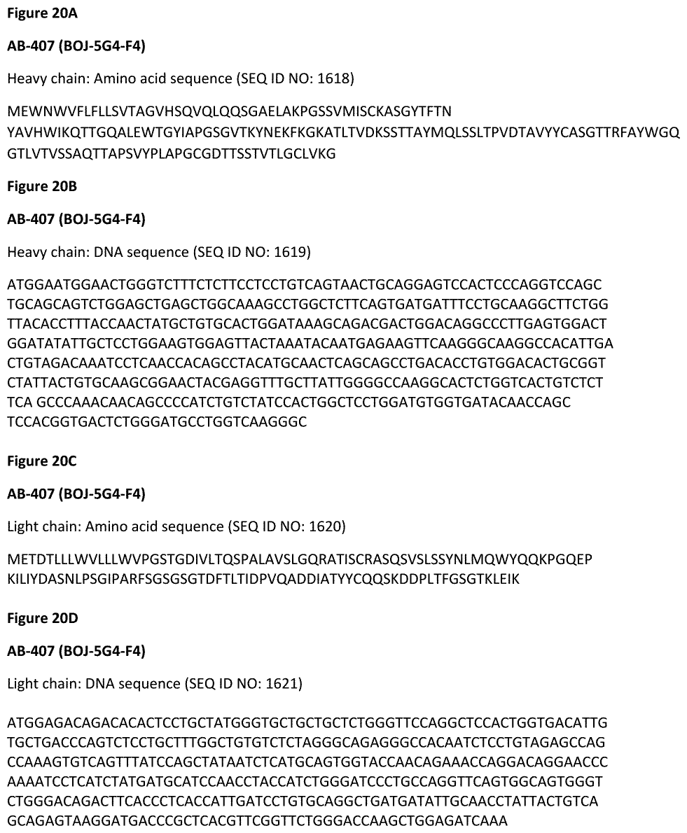

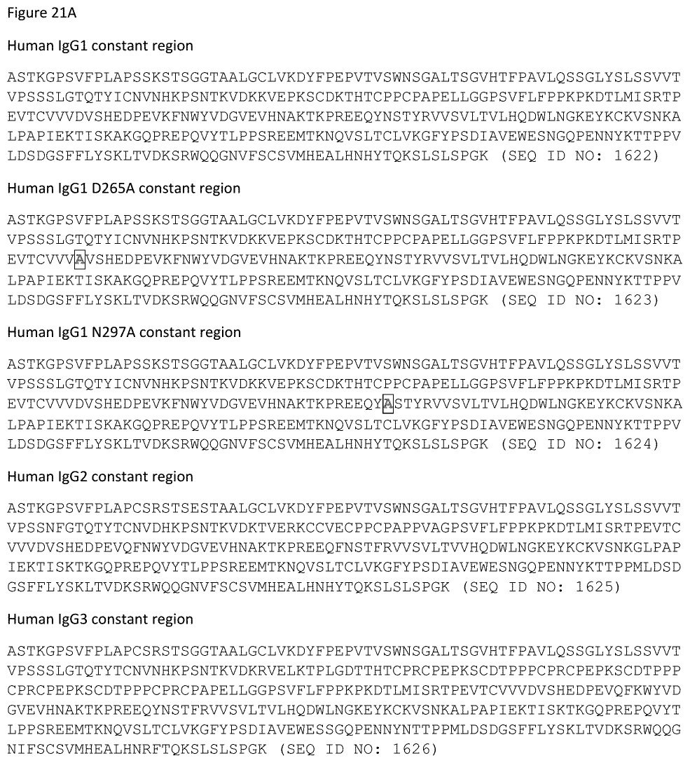

Specific humanized antibodies of CHA antibodies include those shown in FIGS. 11A-11I, FIGS. 12A-12E and FIG. 13. The anti-PVRIG/anti-TIGIT bispecific antibodies of the invention can comprise CHA PVRIG antibody sequences as shown in FIGS. 11A-11I, FIGS. 12A-12E and FIG. 13 as the PVRIG binding portion of the anti-PVRIG/anti-TIGIT bispecific antibodies. As will be appreciated by those in the art, each humanized variable heavy (Humanized Heavy; HH) and variable light (Humanized Light, HL) sequence can be combined with the constant regions of human IgG1, IgG2, IgG3 and IgG4. That is, CHA.7.518.HH1 is the first humanized variable heavy chain, and CHA.7.518.HH1.1 is the full length heavy chain, comprising the “HH1” humanized sequence with a IgG1 constant region (CHA.7.518.HH1.2 is CHA.7.518.HH1 with IgG2, etc.). In some embodiments, anti-PVRIG/anti-TIGIT bispecific antibody comprises the PVRIG sequences provided in FIGS. 4A-4AA, 5A-5H, 7A-7AE, 11A-11I, 12A-12E, 13, 14A-14BX, 15A-15B, 16A-16E, 17A-17C, 26A-26B, and/or 40A-40T as the PVRIG binding portion.

In some embodiments, the anti-PVRIG antibodies of the present invention include anti-PVRIG antibodies wherein the V_{H} and V_{L} sequences of different anti-PVRIG antibodies can be “mixed and matched” to create other anti-PVRIG antibodies. PVRIG binding of such “mixed and matched” antibodies can be tested using the binding assays described above. e.g., ELISAs). In some embodiments, when V_{H} and V_{L} chains are mixed and matched, a V_{H} sequence from a particular V_{H}/V_{L} pairing is replaced with a structurally similar V_{H} sequence. Likewise, in some embodiments, a V_{L} sequence from a particular V_{H}/V_{L} pairing is replaced with a structurally similar V_{L} sequence. For example, the V_{H} and V_{L} sequences of homologous antibodies are particularly amenable for mixing and matching. The anti-PVRIG/anti-TIGIT bispecific antibodies of the invention can comprise PVRIG V_{H} and V_{L} sequences from different anti-PVRIG antibodies that have been “mixed and matched” as the PVRIG binding portion of the anti-PVRIG/anti-TIGIT bispecific antibodies.

Accordingly, the antibodies of the invention comprise CDR amino acid sequences selected from the group consisting of (a) sequences as listed herein; (b) sequences that differ from those CDR amino acid sequences specified in (a) by 1, 2, 3, 4, 5, 6, 7, 8, 9, 10 or more amino acid substitutions; (c) amino acid sequences having 90% or greater, 95% or greater, 98% or greater, or 99% or greater sequence identity to the sequences specified in (a) or (b); (d) a polypeptide having an amino acid sequence encoded by a polynucleotide having a nucleic acid sequence encoding the amino acids as listed herein. The anti-PVRIG/anti-TIGIT bispecific antibodies of the invention can comprise PVRIG variant CDR sequences as part of the PVRIG binding portion of the anti-PVRIG/anti-TIGIT bispecific antibodies.

Additionally included in the definition of PVRIG antibodies are antibodies that share identity to the PVRIG antibodies enumerated herein. That is, in certain embodiments, an anti-PVRIG antibody according to the invention comprises heavy and light chain variable regions comprising amino acid sequences that are homologous to isolated anti-PVRIG amino acid sequences of preferred anti-PVRIG immune molecules, respectively, wherein the antibodies retain the desired functional properties of the parent anti-PVRIG antibodies. The percent identity between the two sequences is a function of the number of identical positions shared by the sequences (e.g., % homology=# of identical positions/total # of positions X 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below. The anti-PVRIG/anti-TIGIT bispecific antibodies of the invention can comprise heavy and light chain variable regions comprising amino acid sequences that are homologous to isolated anti-PVRIG amino acid sequences as described herein.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J Mol. Biol. 215:403-10. BLAST protein searches can be performed with the XBLAST program, score=50, wordlength=3 to obtain amino acid sequences homologous to the antibody molecules according to at least some embodiments of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In general, the percentage identity for comparison between PVRIG antibodies is at least 75%, at least 80%, at least 90%, with at least about 95%, 96%, 97%, 98%, or 99% percent identity being preferred. The percentage identity may be along the whole amino acid sequence, for example the entire heavy or light chain or along a portion of the chains. For example, included within the definition of the anti-PVRIG antibodies of the invention are those that share identity along the entire variable region (for example, where the identity is 95% or 98% identical along the variable regions), or along the entire constant region, or along just the Fc domain. In particular, the invention provides anti-PVRIG/anti-TIGIT bispecific antibodies that have PVRIG binding portions or antigen binding domains with at least 75%, at least 80%, at least 90%, with at least about 95%, 96%, 97%, 98%, or 99% percent identity being preferred, with the CHA.7.518.4 antibody.

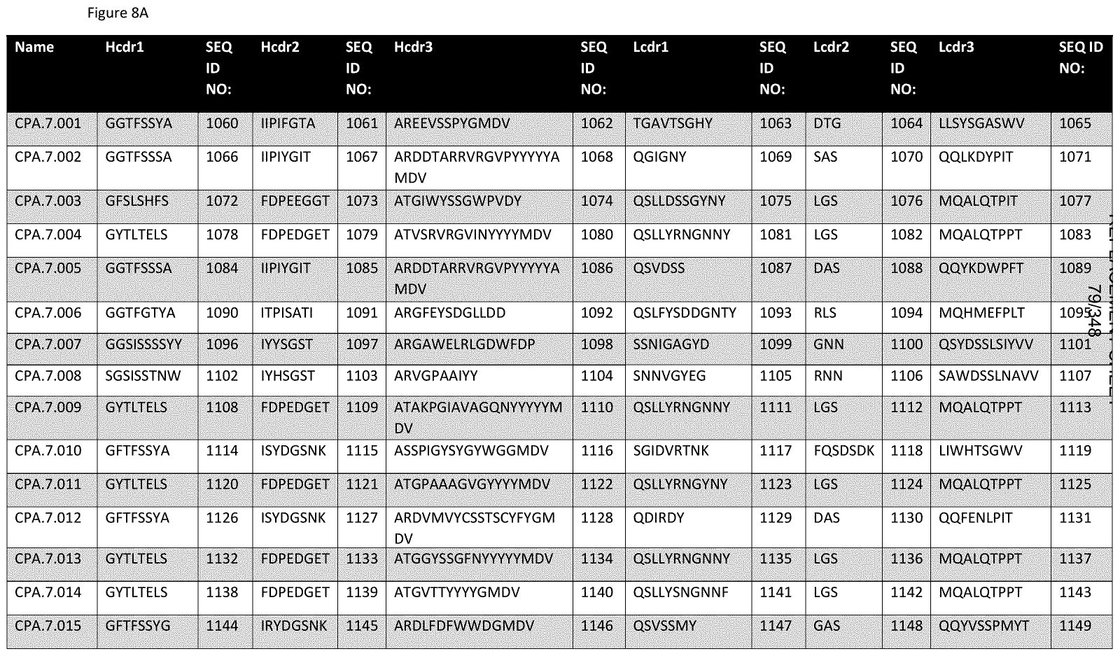

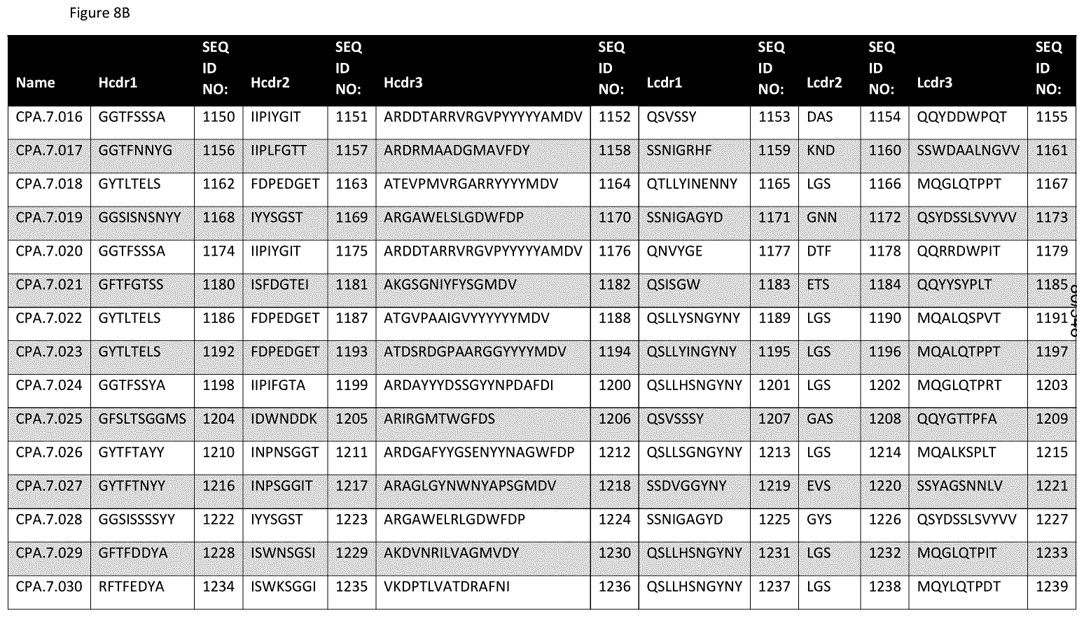

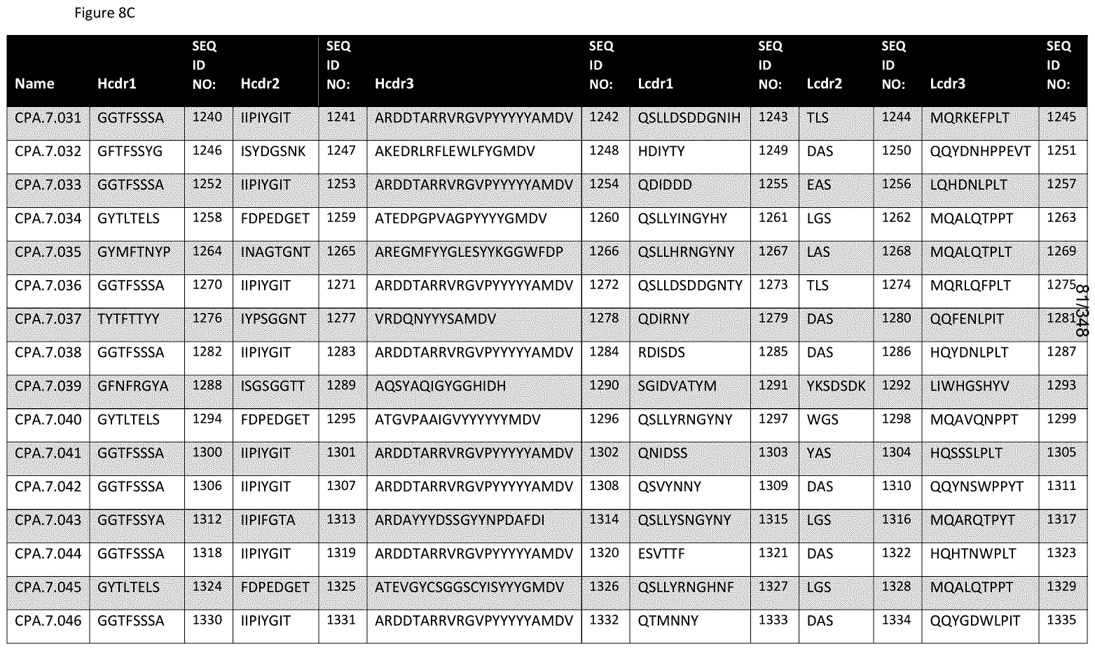

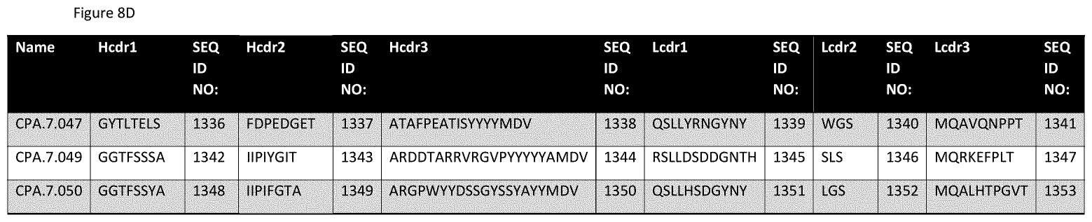

In addition, also included are sequences that may have the identical CDRs but changes in the variable domain (or entire heavy or light chain). For example, PVRIG antibodies include those with CDRs identical to those shown in FIGS. 8A-8D but whose identity along the variable region can be lower, for example 95 or 98% percent identical. In particular, the invention provides anti-PVRIG/anti-TIGIT bispecific antibodies that have PVRIG binding portions or antigen binding domains with identical CDRs to CHA.7.518.4 but with framework regions that are 95% or 98% identical to CHA.7.518.4.

The anti-PVRIG/anti-TIGIT bispecific antibodies of the invention can comprise CDRs identical to those shown in FIGS. 8A-8D as part of the PVRIG binding portion of the anti-PVRIG/anti-TIGIT bispecific antibodies.

In addition, also included are sequences that may have the identical CDRs but changes in the variable domain (or entire heavy or light chain). For example, PVRIG antibodies include those with CDRs identical to those shown in FIGS. 35A-35D but whose identity along the variable region can be lower, for example 95 or 98% percent identical.

The anti-PVRIG/anti-TIGIT bispecific antibodies of the invention can comprise CDRs identical to those shown in FIGS. 35A-35D as part of the PVRIG binding portion of the anti-PVRIG/anti-TIGIT bispecific antibodies.

In some embodiments, the PVRIG binding portion is from an anti-PVRIG antibody as provided in WO 2017/041004 (incorporated herein by reference in its entirety). In some embodiments, the PVRIG binding portion is from an anti-PVRIG antibody as provided in WO 2018/017864 (incorporated herein by reference in its entirety).

1. PVRIG Antibodies that Compete for Binding with Enumerated Antibodies

The present invention provides not only the enumerated antibodies but additional antibodies that compete with the enumerated antibodies (the CPA and CHA numbers enumerated herein that specifically bind to PVRIG) to specifically bind to the PVRIG molecule. The PVRIG antibodies of the invention “bin” into different epitope bins. There are four separate bins outlined herein; 1) the epitope bin into which CPA.7.002, CPA.7.003, CPA.7.005, CPA.7.007, CPA.7.010, CPA.7.012, CPA.7.015, CPA.7.016, CPA.7.017, CPA.7.019, CPA.7.020, CPA.7.021, CPA.7.024, CPA.7.028, CPA.7.032, CPA.7.033, CPA.7.036, CPA.7.037, CPA.7.038, CPA.7.043, CPA.7.046 and CPA.7.041 all fall into; 2) the epitope bin into which CPA.7.004, CPA.7.009, CPA.7.011, CPA.7.014, CPA.7.018, CPA.7.022, CPA.7.023, CPA.7.034, CPA.7.040, CPA.7.045 and CPA.7.047 all fall into; 3) CPA.7.039, which defines the distinction between bin 1 and bin 2, in that bin 1 blocks CPA.7.039 binding and bin 2 sandwiches the ligand with CPA.7.039, and bin 4) with CPA.7.050. The anti-PVRIG/anti-TIGIT bispecific antibodies of the invention can comprise PVRIG antibodies and/or antigen binding domains sequences that are capable of competing with the enumerated antibodies (the CPA and CHA numbers enumerated herein that specifically bind to PVRIG) as part of the PVRIG binding portion of the anti-PVRIG/anti-TIGIT bispecific antibodies.

Thus, the invention provides anti-PVRIG/anti-TIGIT bispecific antibodies, where the PVRIG binding portion of the anti-PVRIG/anti-TIGIT bispecific antibodies is capable of competing for binding with antibodies that are in bin 1, with antibodies that are in bin 2, with antibodies that are in bin 3 and/or with antibodies that are in bin 4.

Additional anti-PVRIG/anti-TIGIT bispecific antibodies that compete with the enumerated antibodies are generated, as is known in the art and generally outlined below. Competitive binding studies can be done as is known in the art, generally using SPR/Biacore® binding assays, as well as ELISA and cell-based assays.

C. TIGIT Binding Portion of the Anti-PVRIG/Anti-TIGIT Bispecific Antibodies

The anti-TIGIT and/or anti-PVRIG/anti-TIGIT bispecific antibodies described herein can comprise a TIGIT antibody and/or antigen binding domain sequence as part of the TIGIT binding portion, where the TIGIT antibodies are labeled as follows. Such TIGIT antibodies have reference numbers, for example “CPA.9.086”. This represents the combination of the variable heavy and variable light chains, as depicted in FIG. 23A-23D, for example, with the understanding that these antibodies include two heavy chains and two light chains. “CPA.9.086.VH” refers to the variable heavy portion of CPA.9.086, while “CPA.9.086.VL” is the variable light chain. “CPA.9.086.vhCDR1”, “CPA.9.086.vhCDR2”, “CPA.9.086.vhCDR3”, “CPA.9.086.vlCDR1”, “CPA.9.086.vlCDR2”, and “CPA.9.086.vlCDR3”, refers to the CDRs are indicated. “CPA.9.086.HC” refers to the entire heavy chain (e.g. variable and constant domain) of this molecule, and “CPA.9.086.LC” refers to the entire light chain (e.g. variable and constant domain) of the same molecule. In general, the human kappa light chain is used for the constant domain of each phage (or humanized hybridoma) antibody herein, although in some embodiments the lambda light constant domain is used. “CPA.9.086.H1” refers to a full length antibody comprising the variable heavy and light domains, including the constant domain of Human IgG1 (hence, the H1; IgG1, IgG2, IgG3 and IgG4 sequences are shown in FIGS. 9A-9C and 21A-21B). Accordingly, “CPA.9.086.H2” would be the CPA.9.086 variable domains linked to a Human IgG2. “CPA.9.086.H3” would be the CPA.9.086 variable domains linked to a Human IgG3, and “CPA.9.086.H4” would be the CPA.9.086 variable domains linked to a Human IgG4. Note that in some cases, the human IgGs may have additional mutations, such are described below, and this can be annotated. For example, in many embodiments, there may be a S241P mutation in the human IgG4, and this can be annotated as “CPA. 9.086.H4(S241P)” for example. The human IgG4 sequence with this S241P hinge variant is shown in FIG. 21A-21B. Other potential variants are IgG1(N297A), (or other variants that ablate glycosylation at this site and thus many of the effector functions associated with FcγRIIIa binding), and IgG1(D265A), which reduces binding to FcγR receptors. The anti-TIGIT and/or anti-PVRIG/anti-TIGIT bispecific antibodies of the invention can comprise any of the TIGIT antibody domain sequences as the TIGIT binding portion of the anti-TIGIT and/or anti-PVRIG/anti-TIGIT bispecific antibodies. The anti-TIGIT and/or anti-PVRIG/anti-TIGIT bispecific antibodies of the invention can comprise any of the TIGIT antigen binding domains as the TIGIT binding portion of the anti-TIGIT and/or anti-PVRIG/anti-TIGIT bispecific antibodies.

The invention further provides variable heavy and light domains as well as full length heavy and light chains.

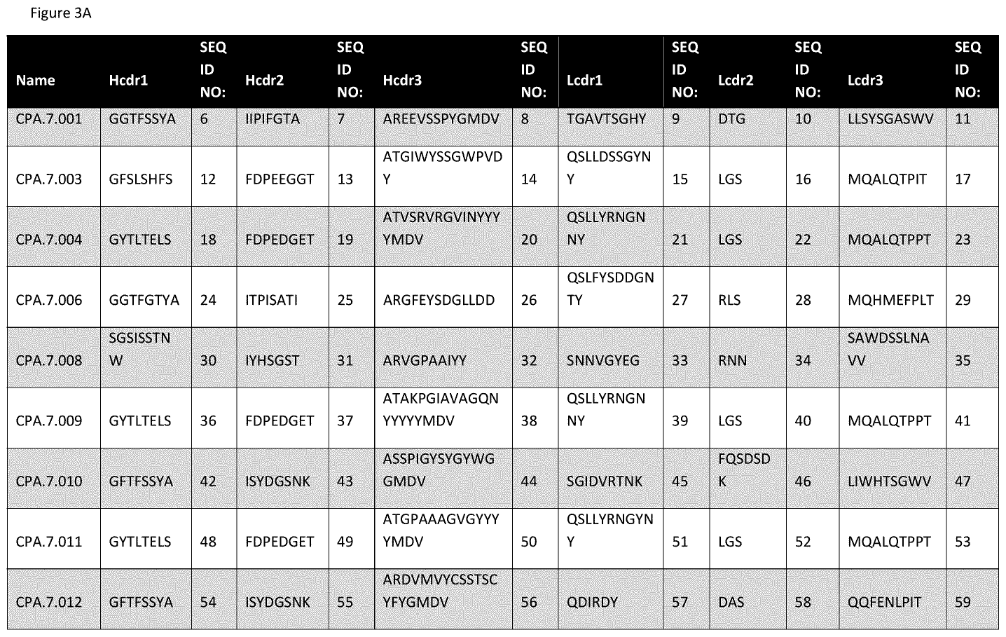

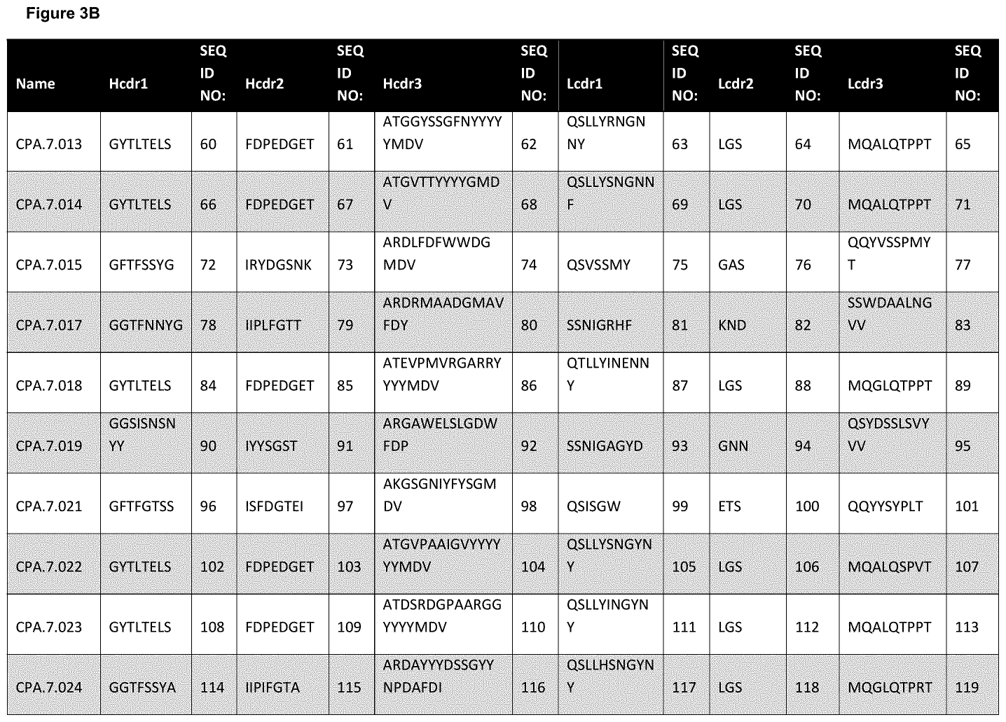

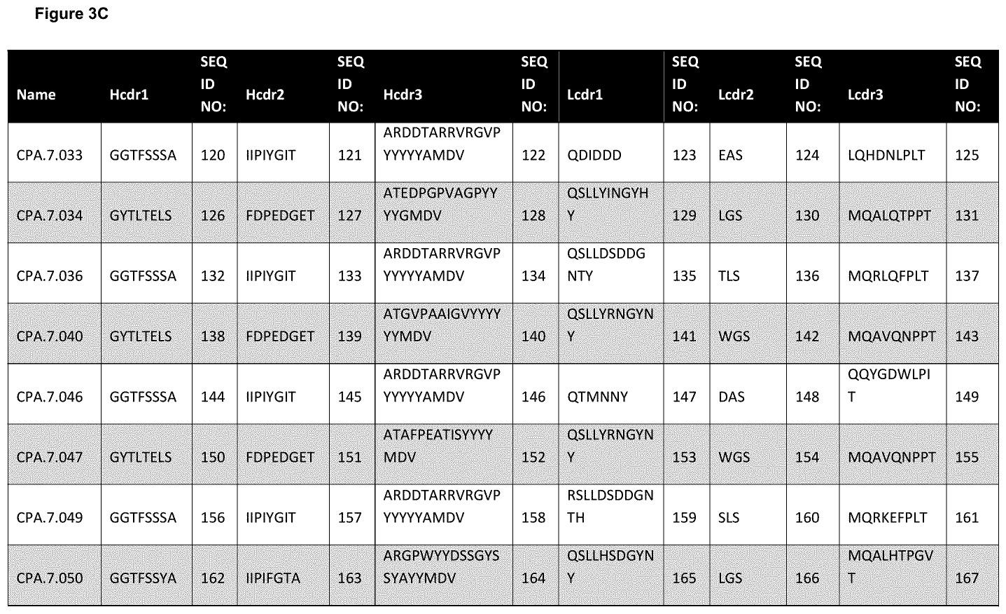

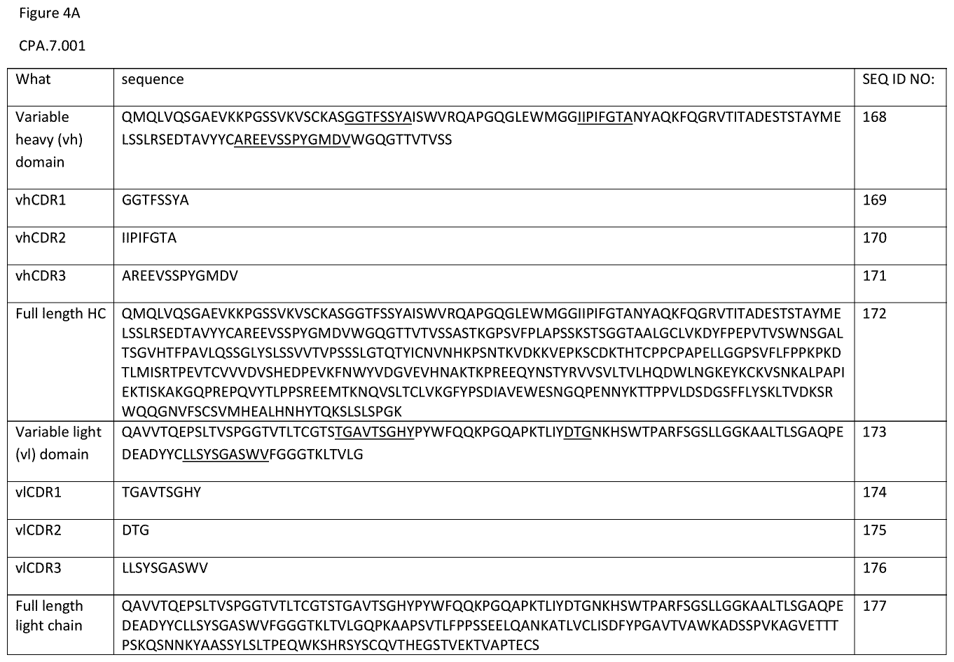

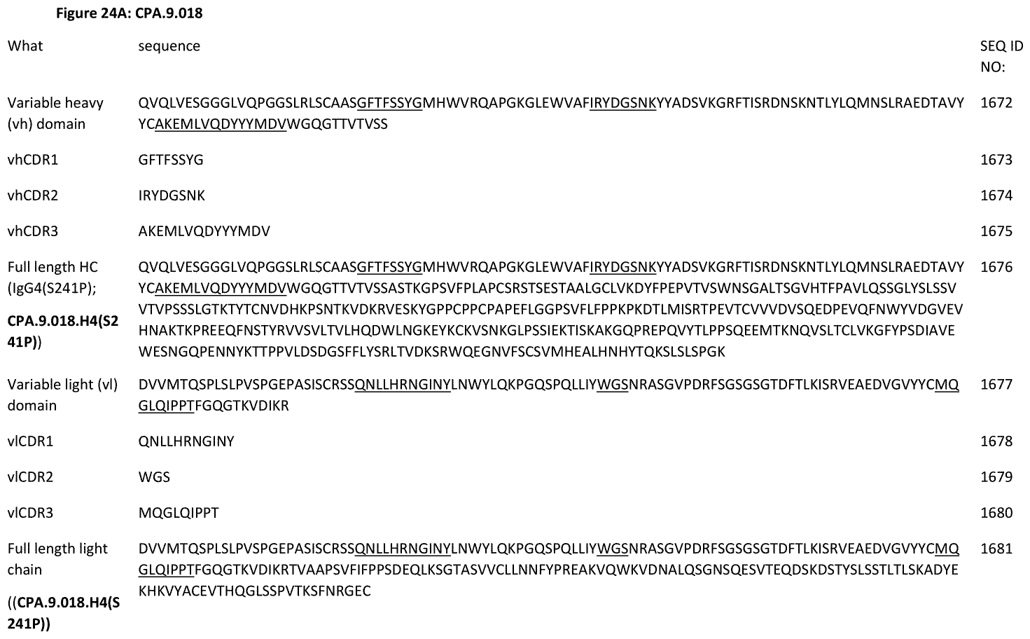

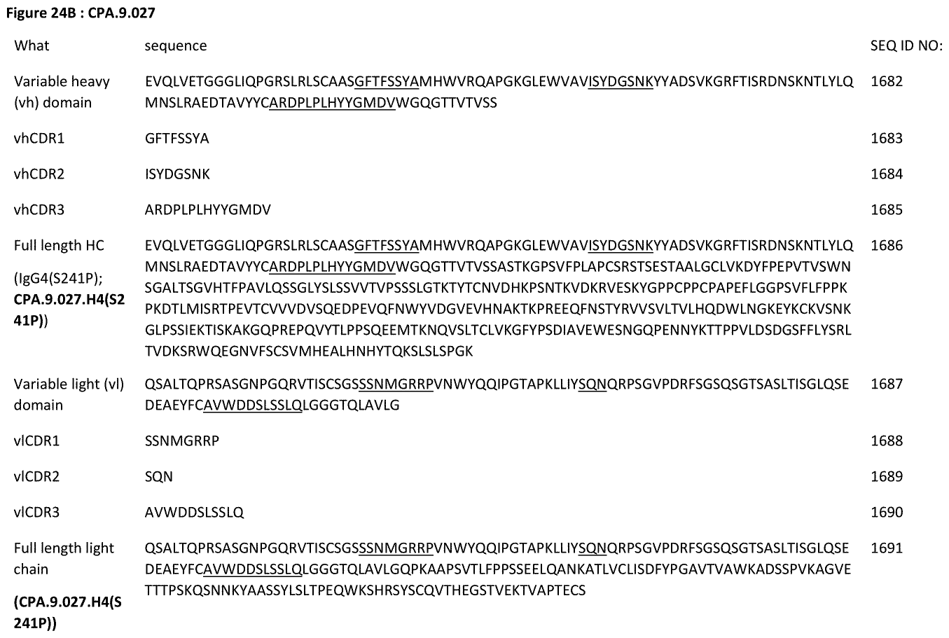

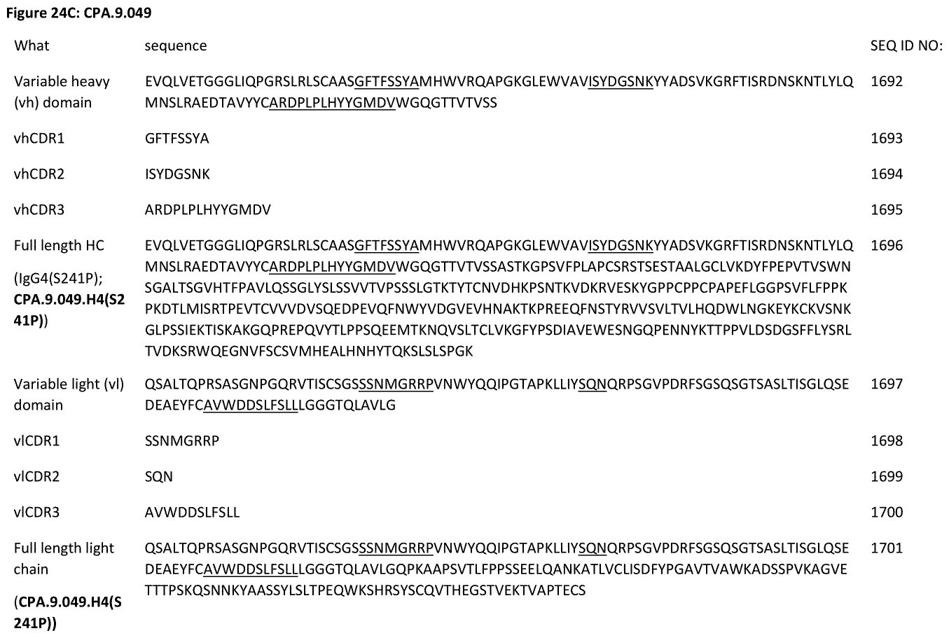

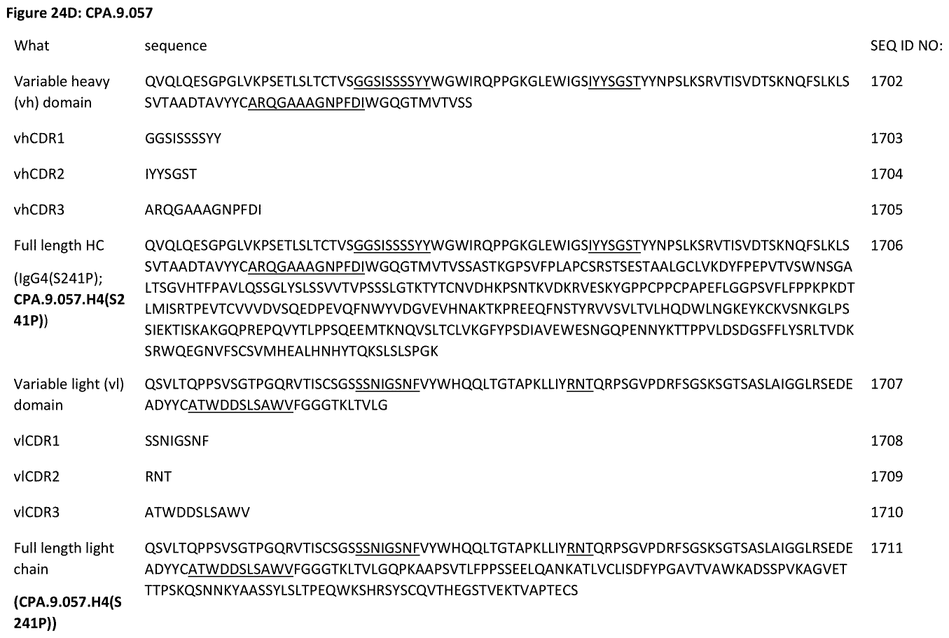

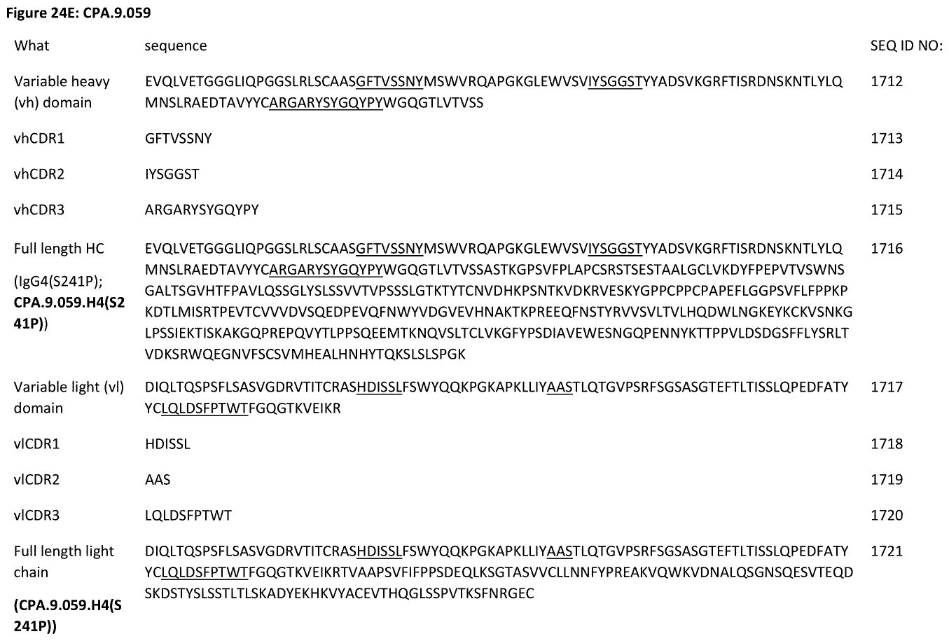

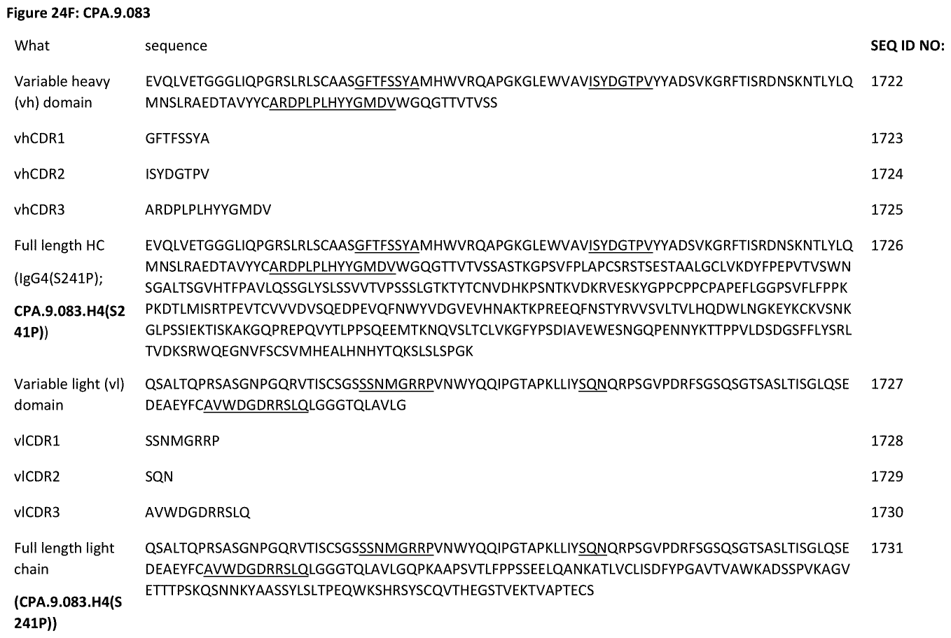

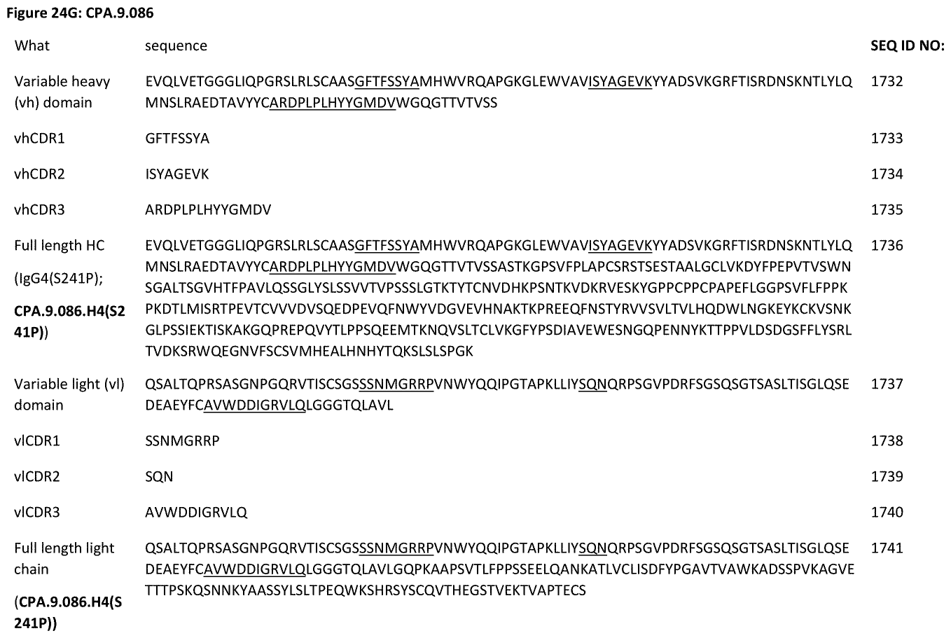

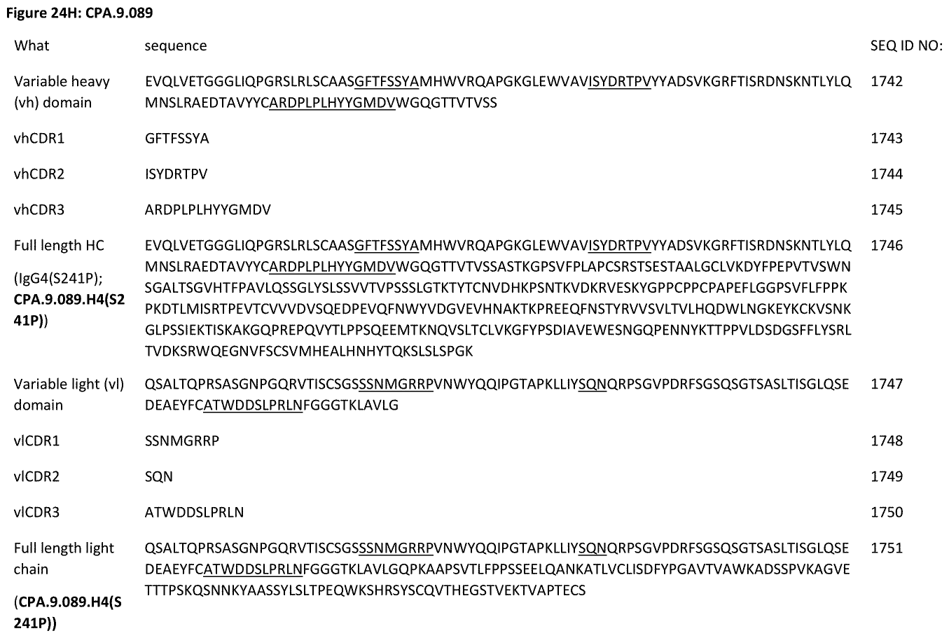

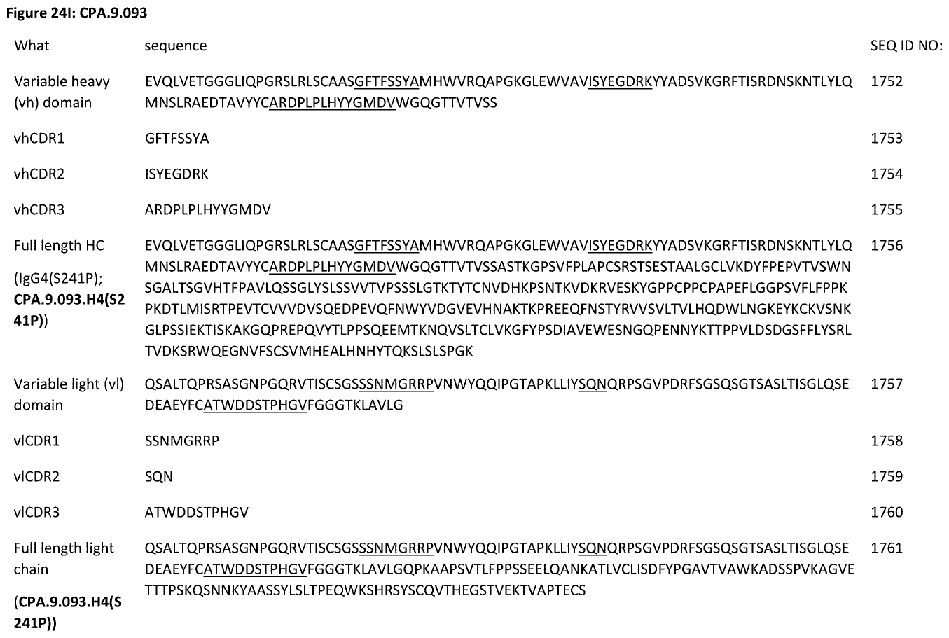

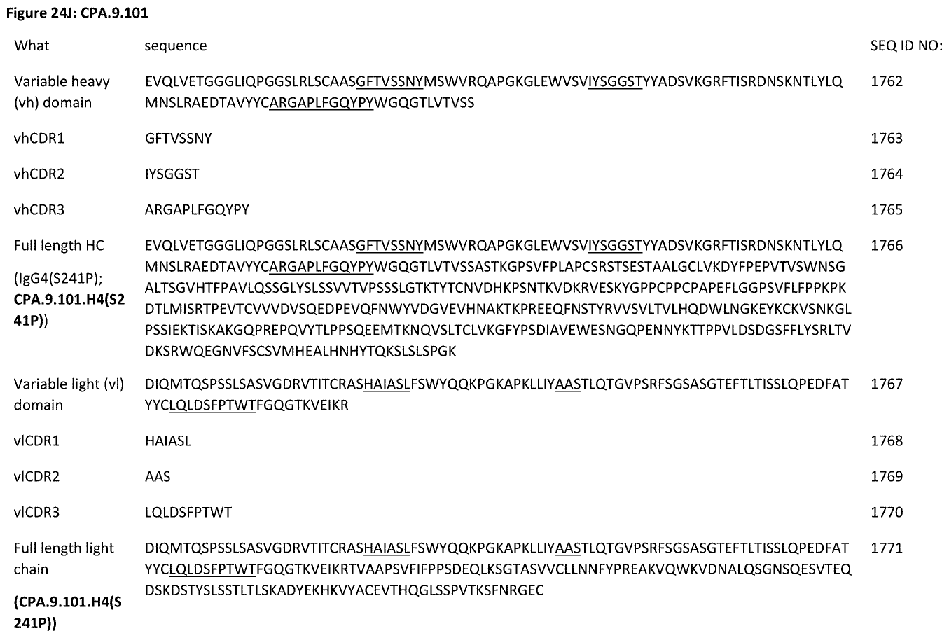

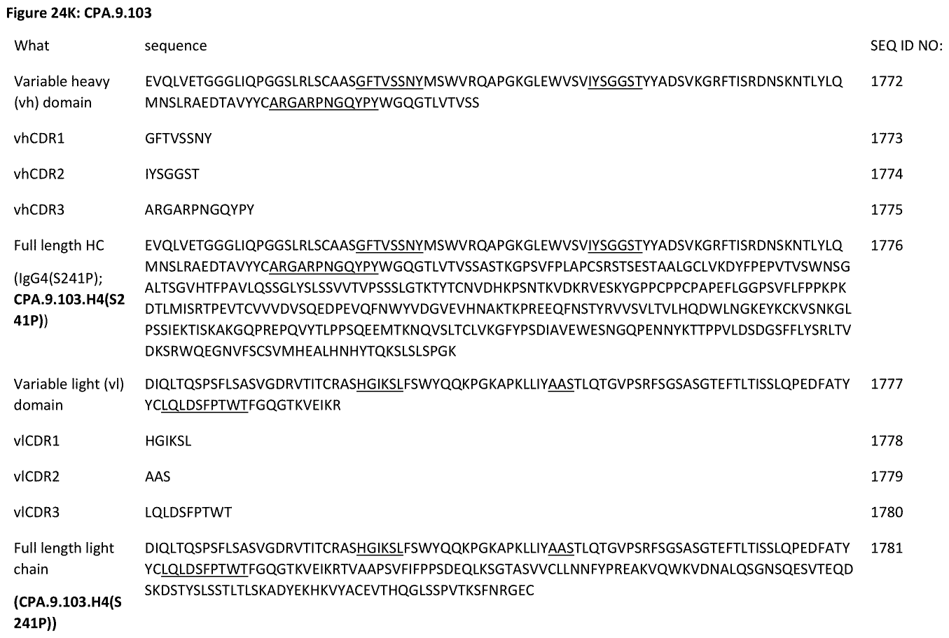

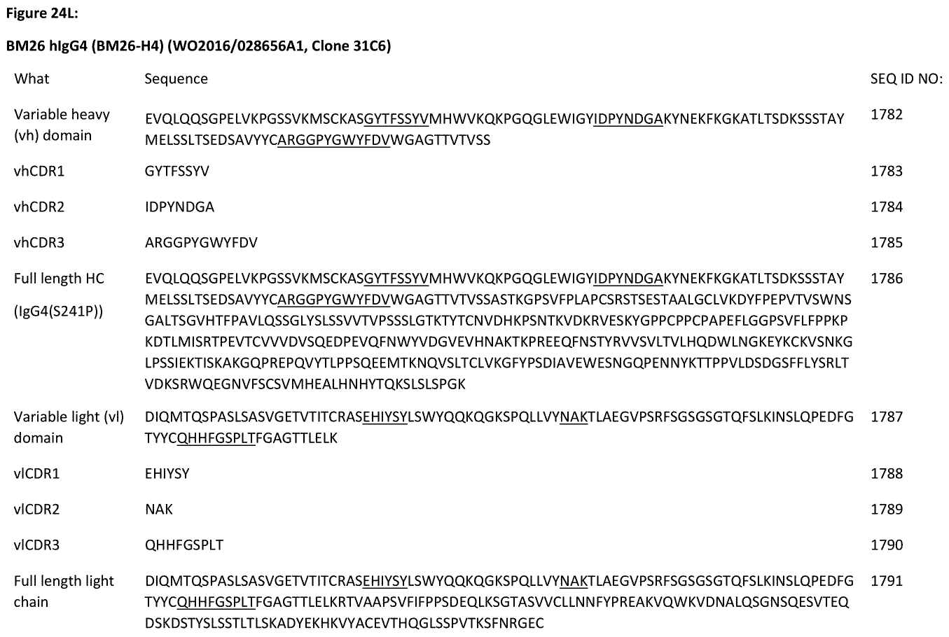

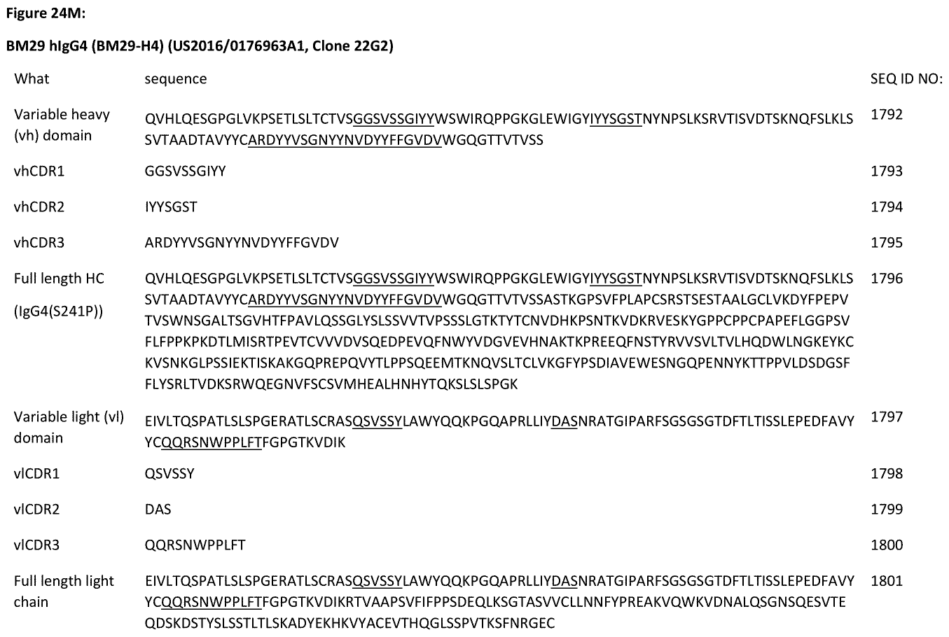

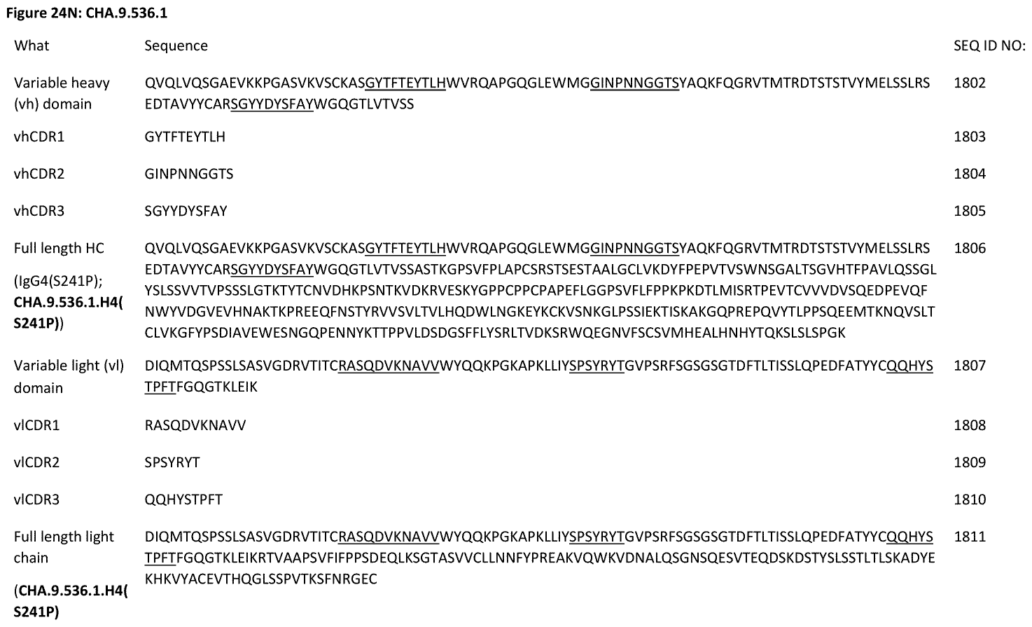

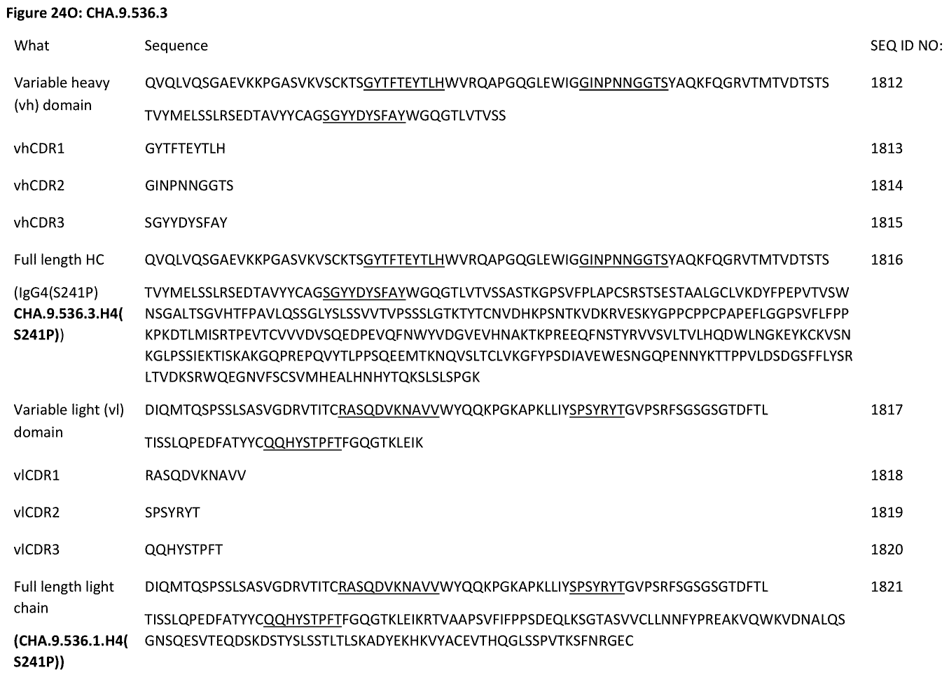

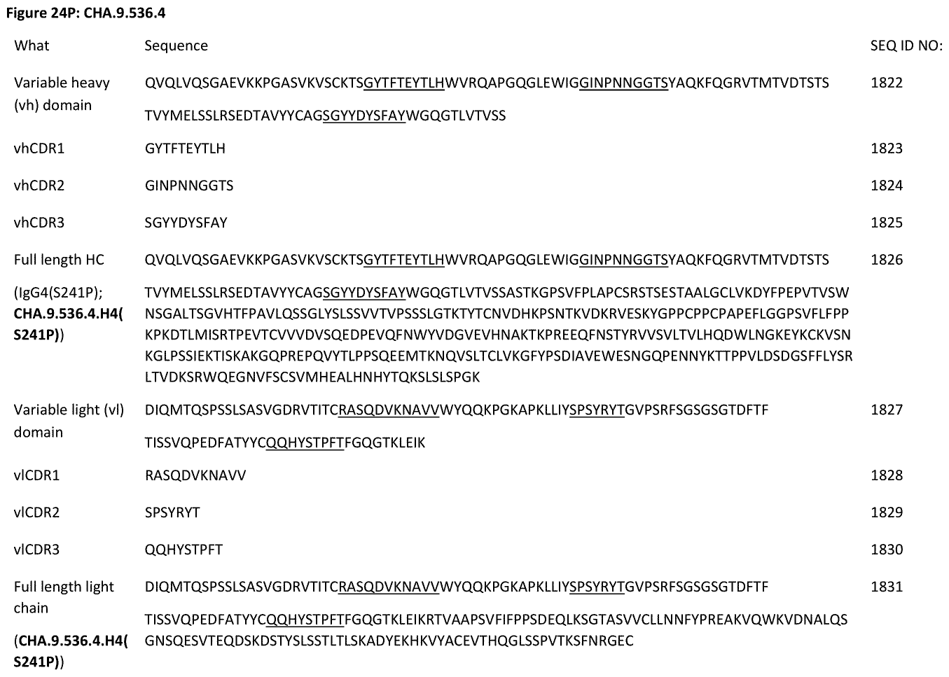

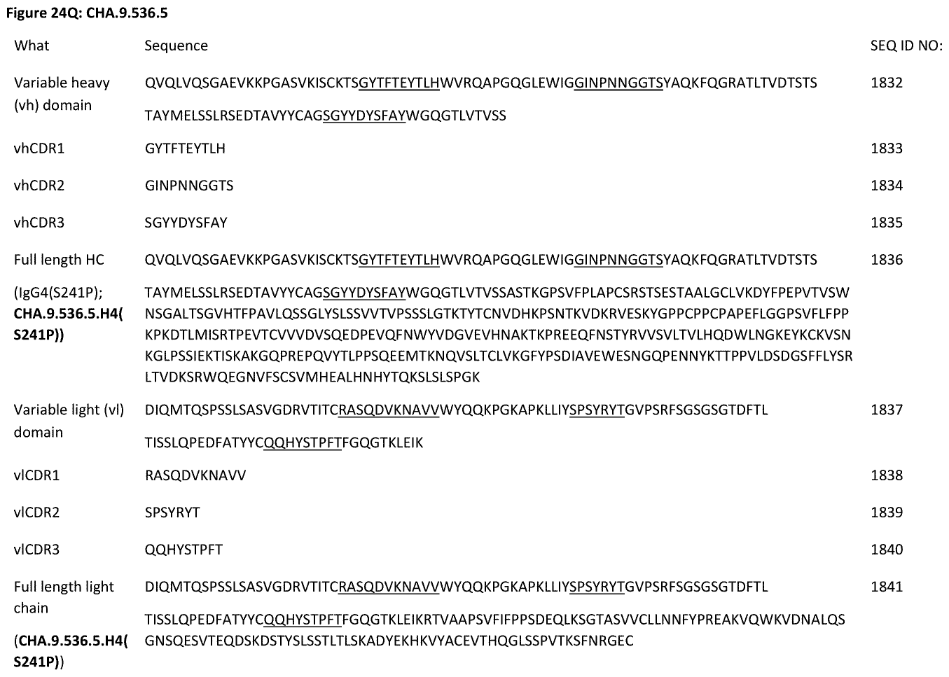

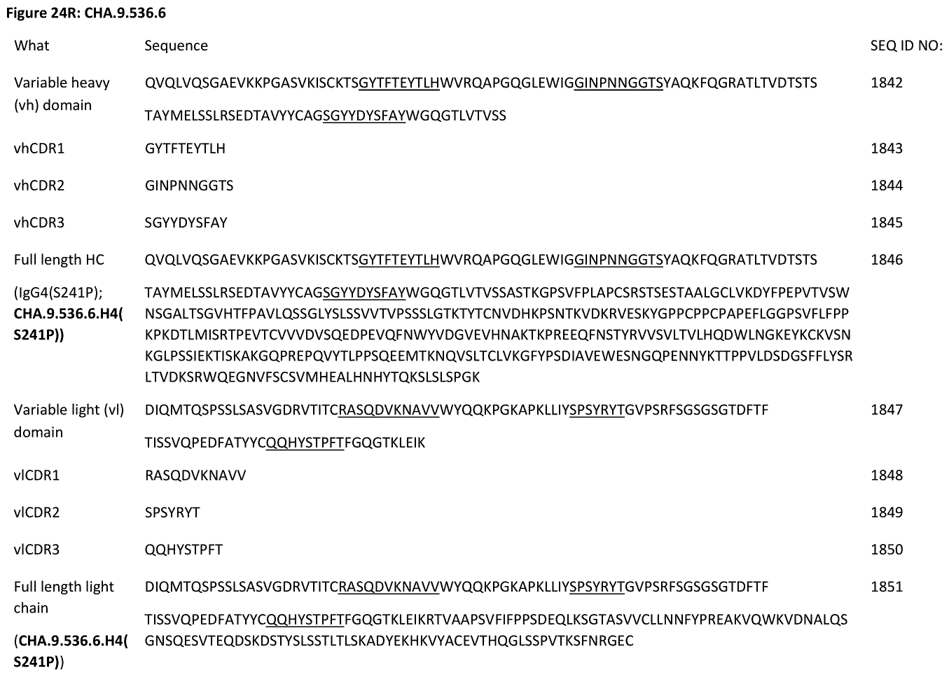

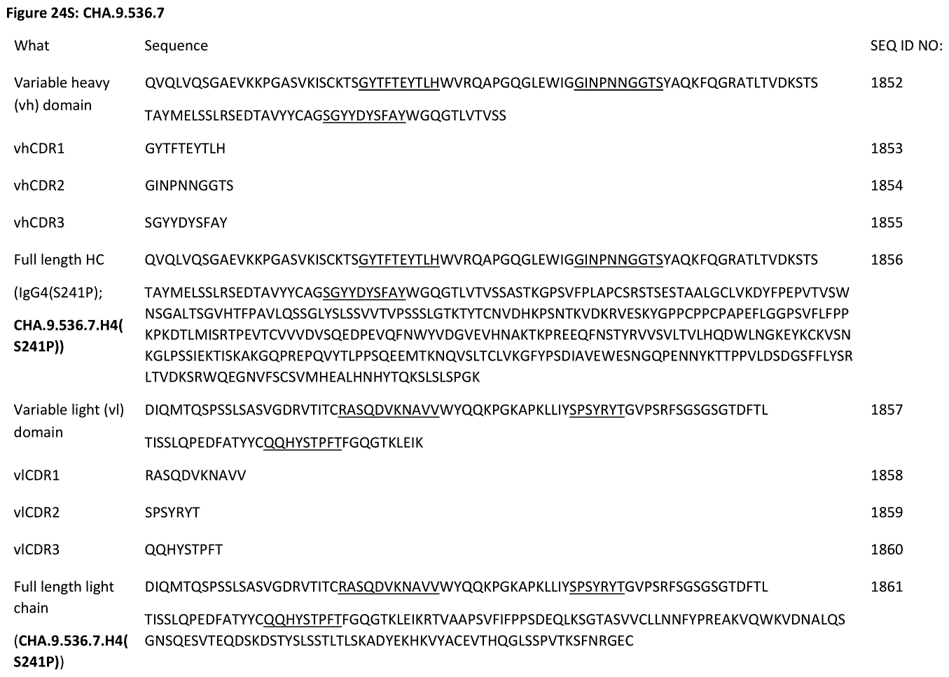

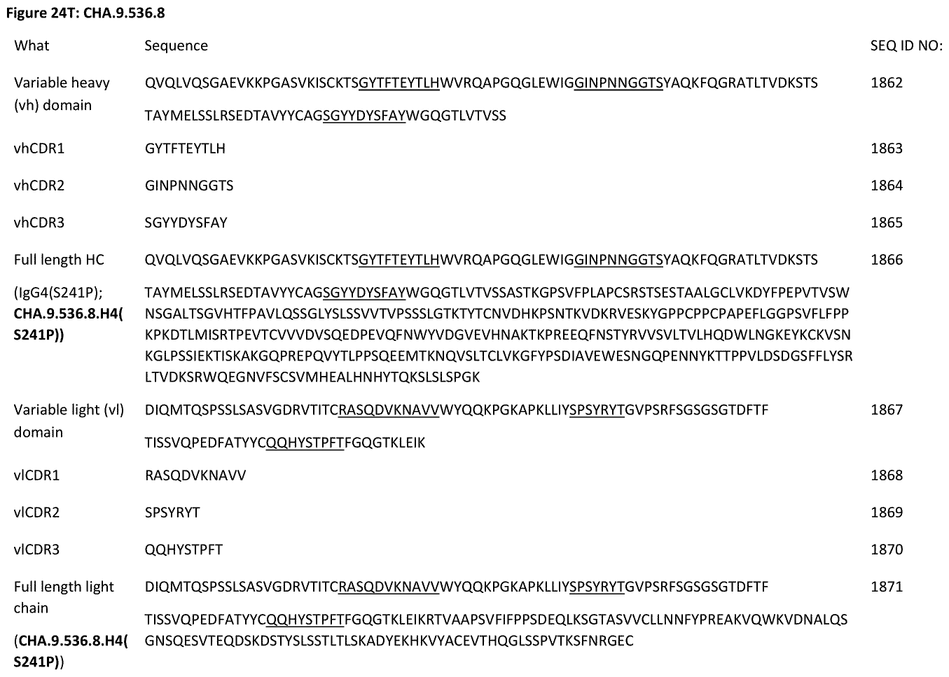

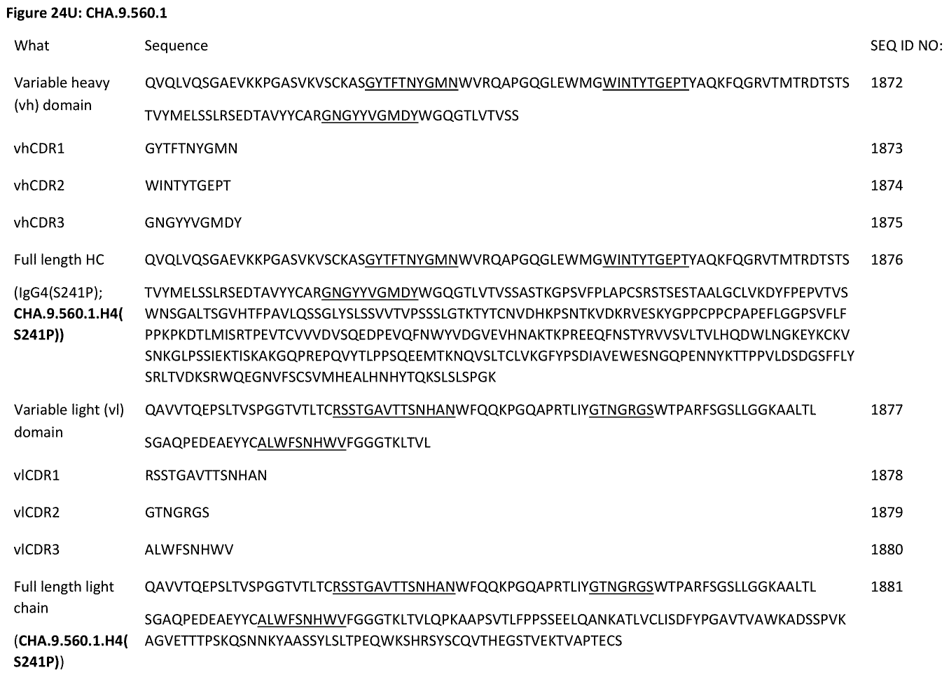

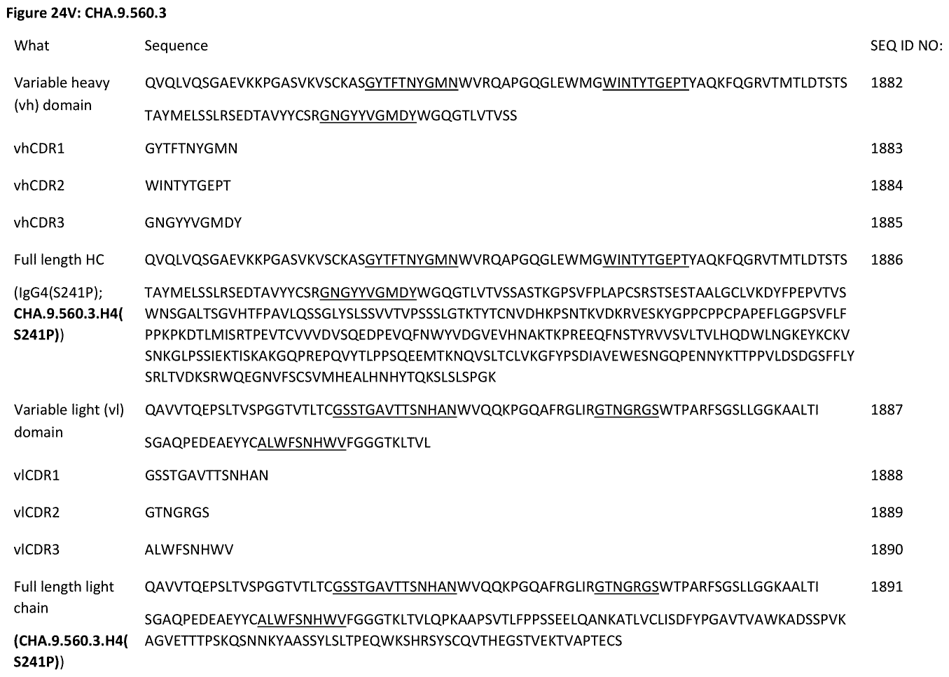

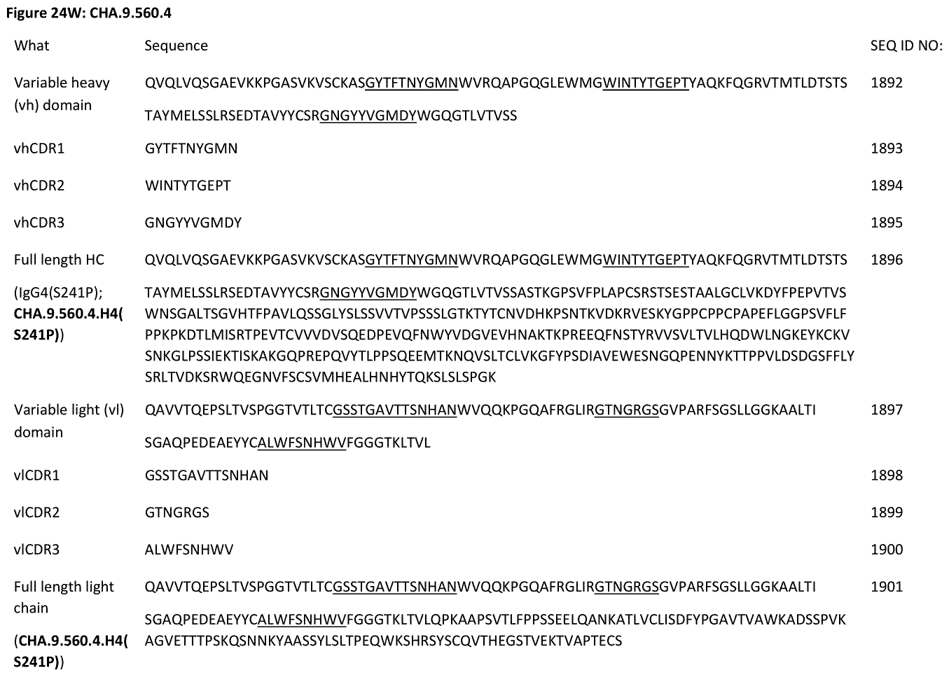

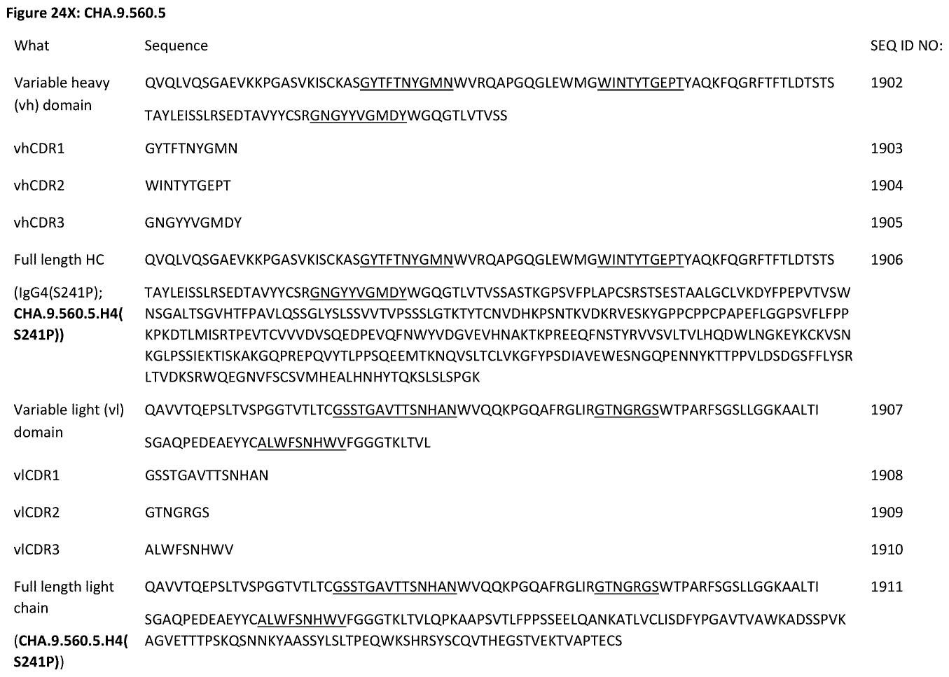

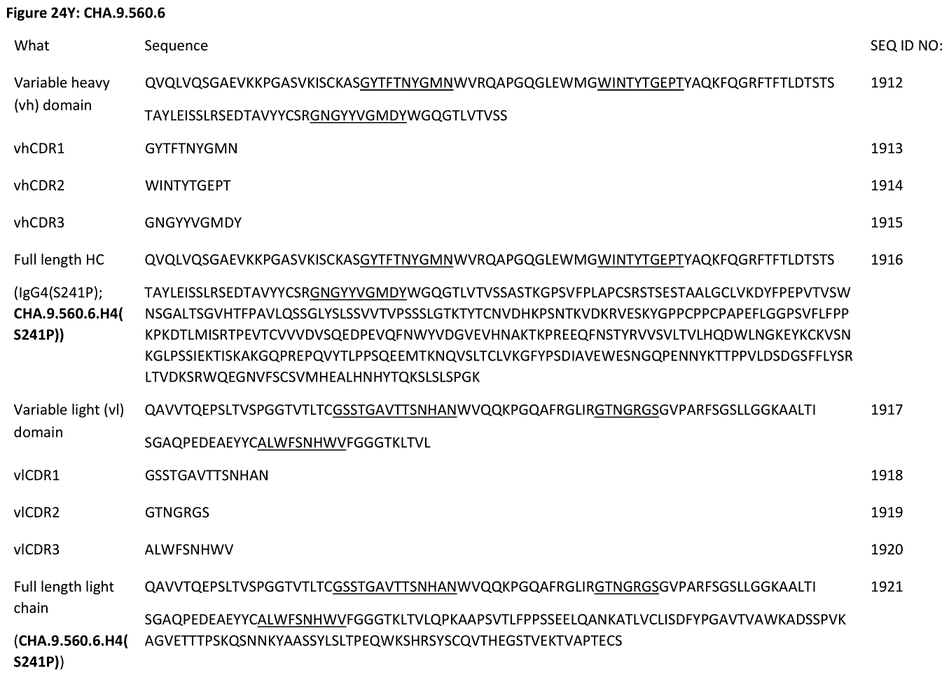

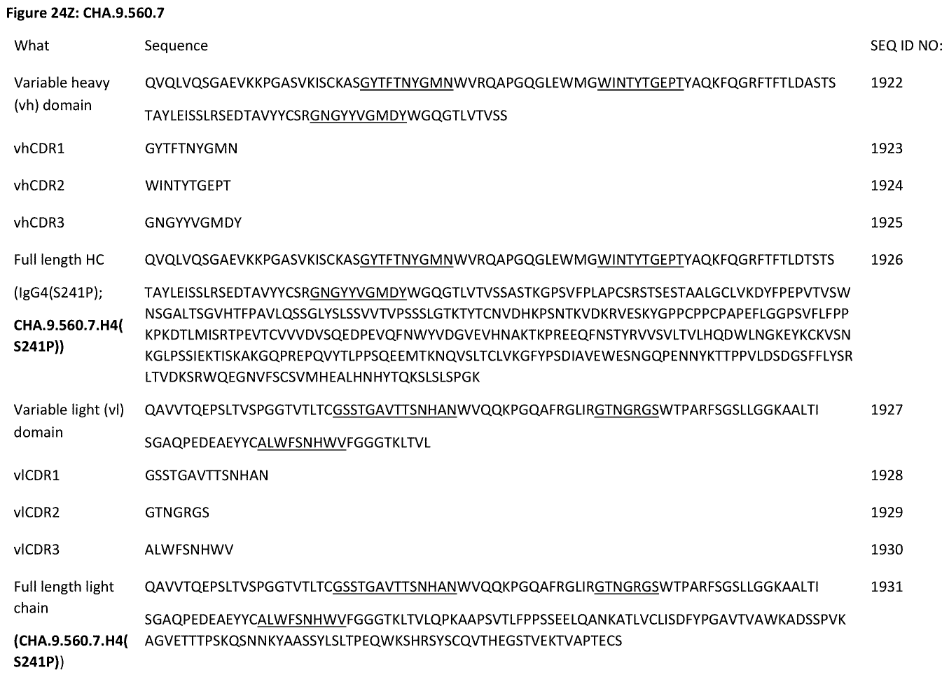

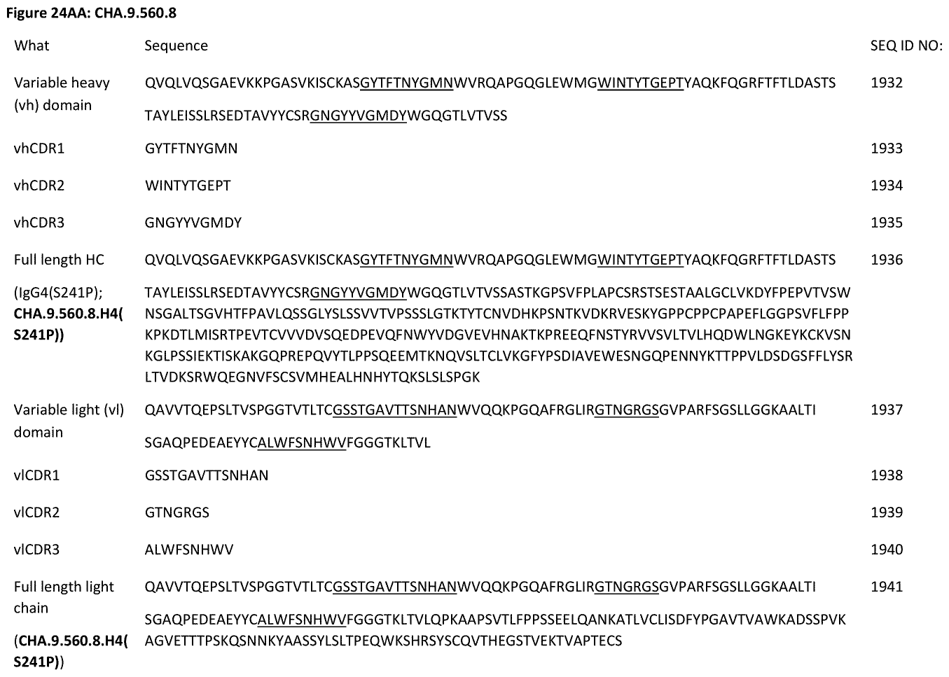

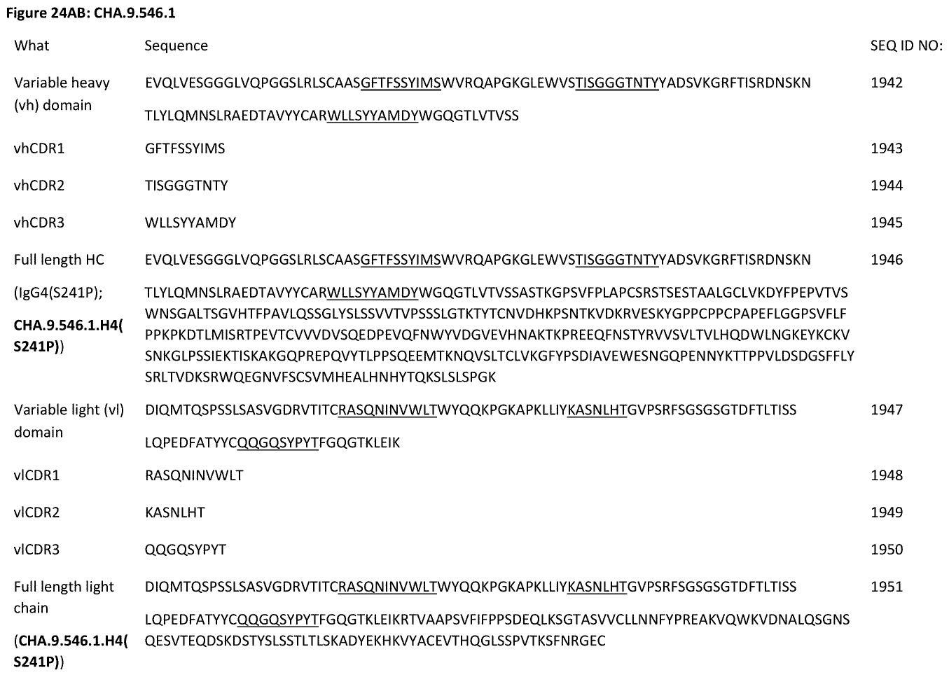

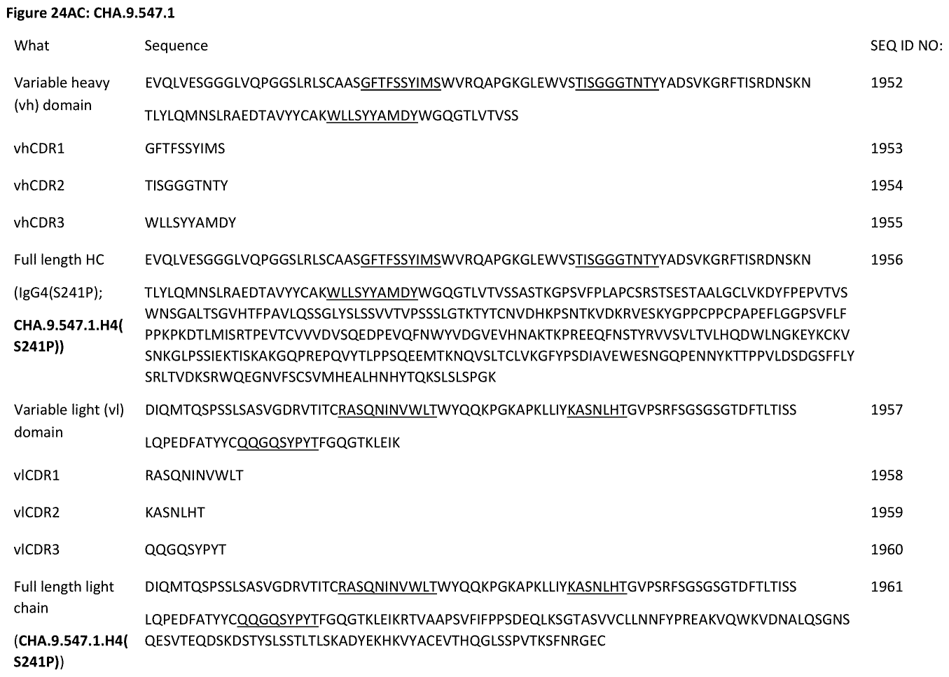

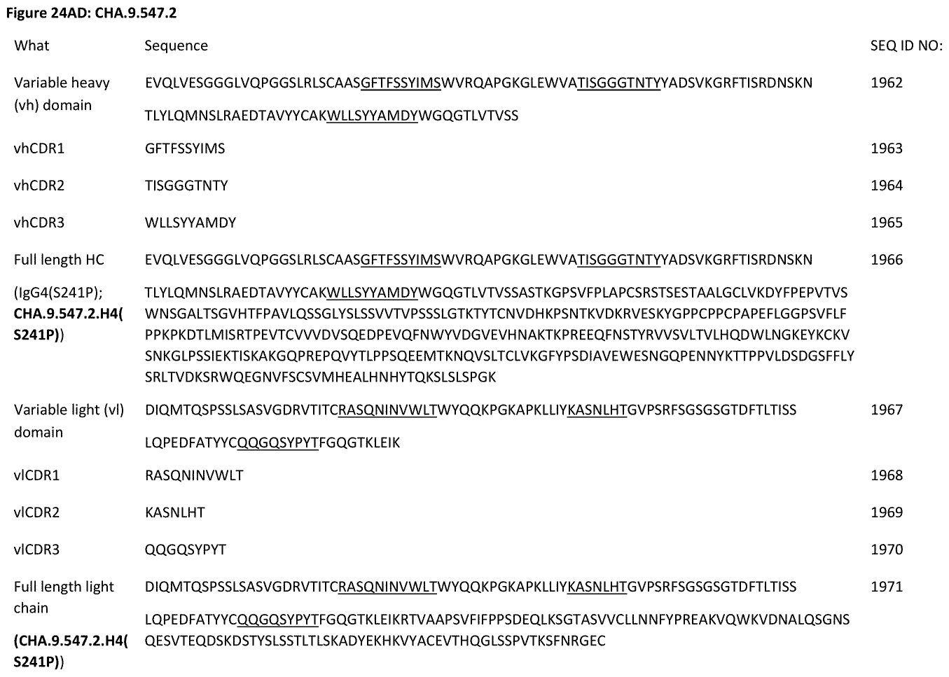

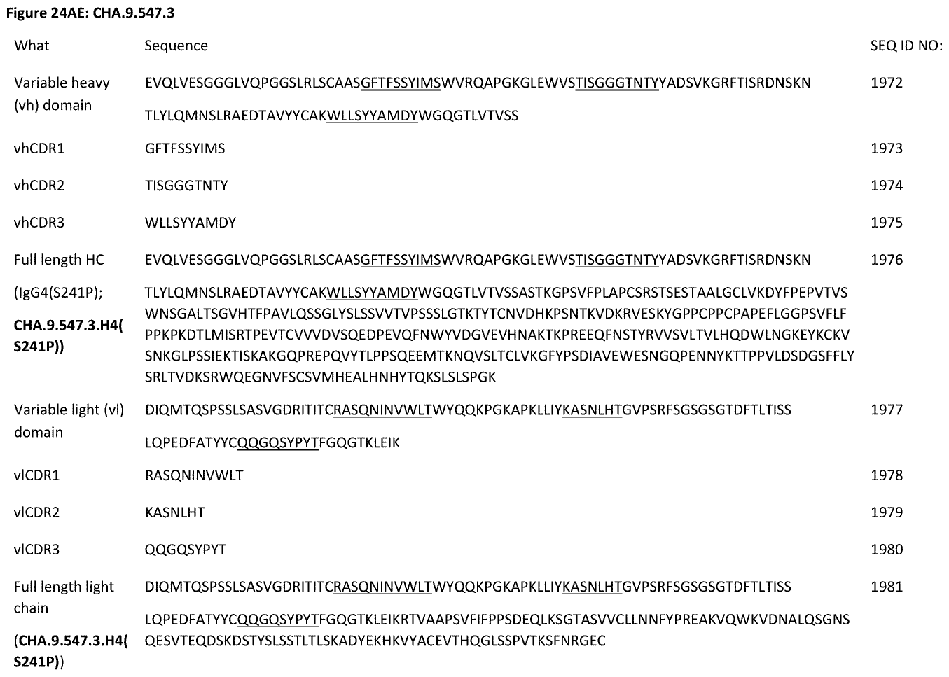

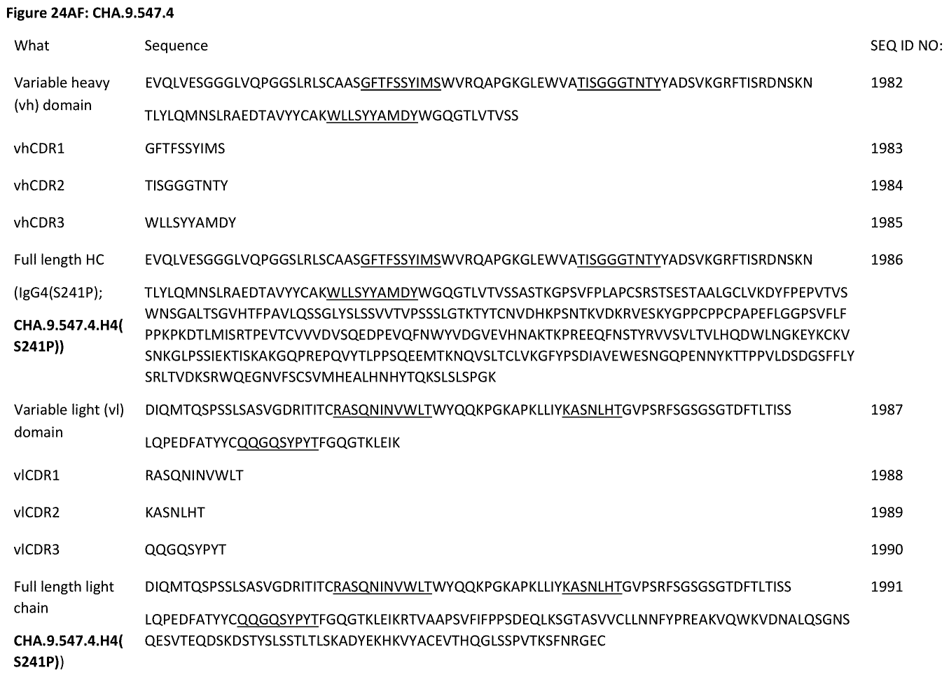

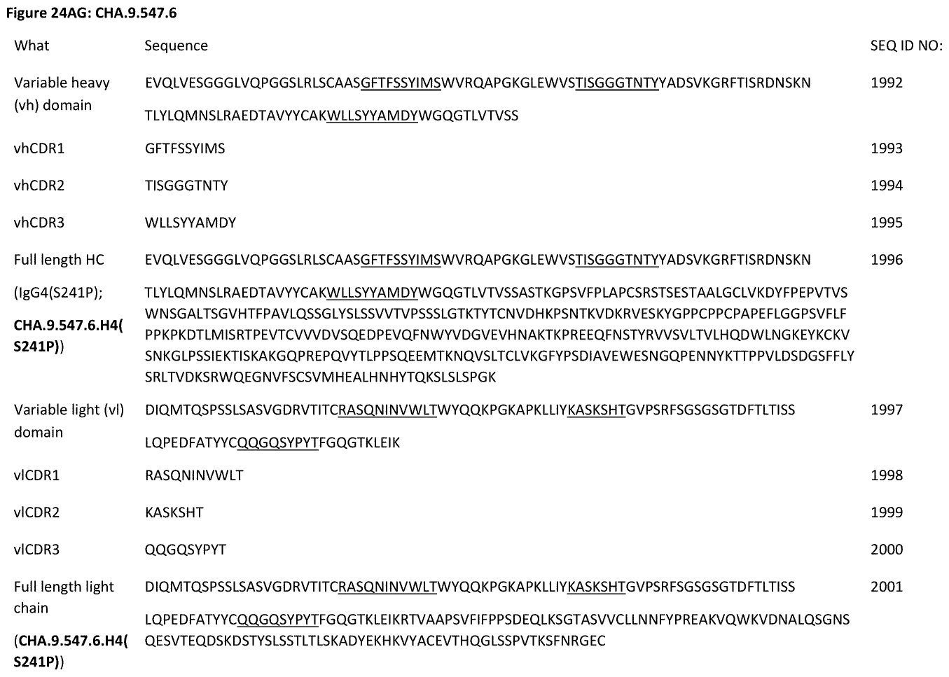

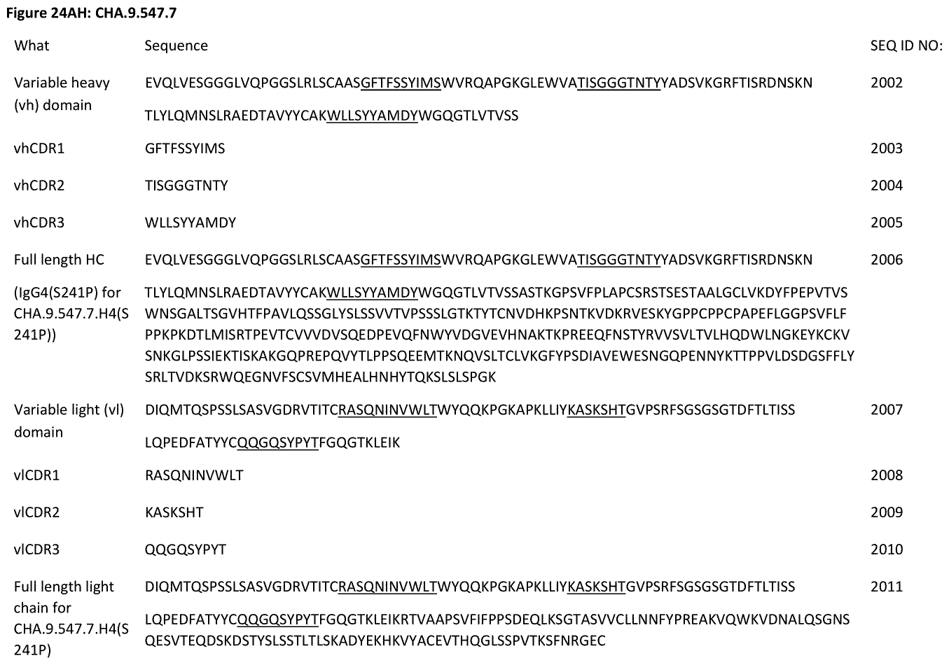

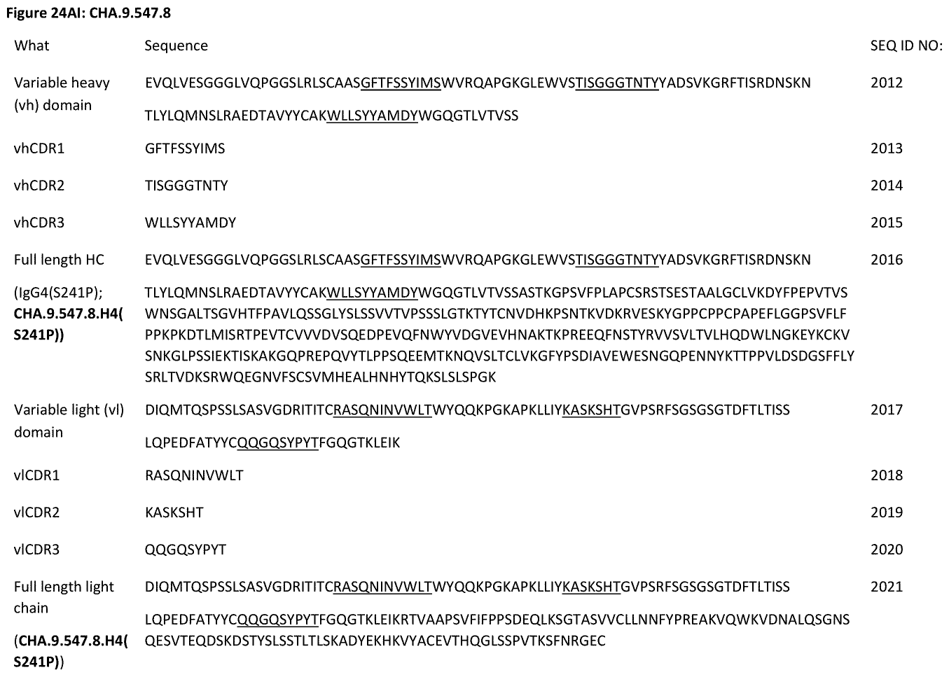

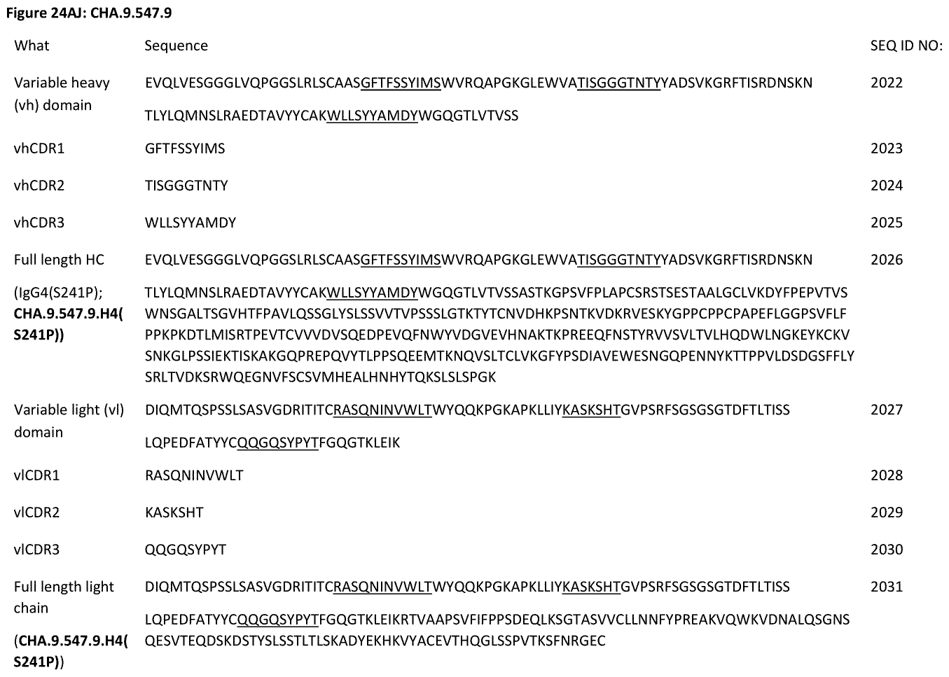

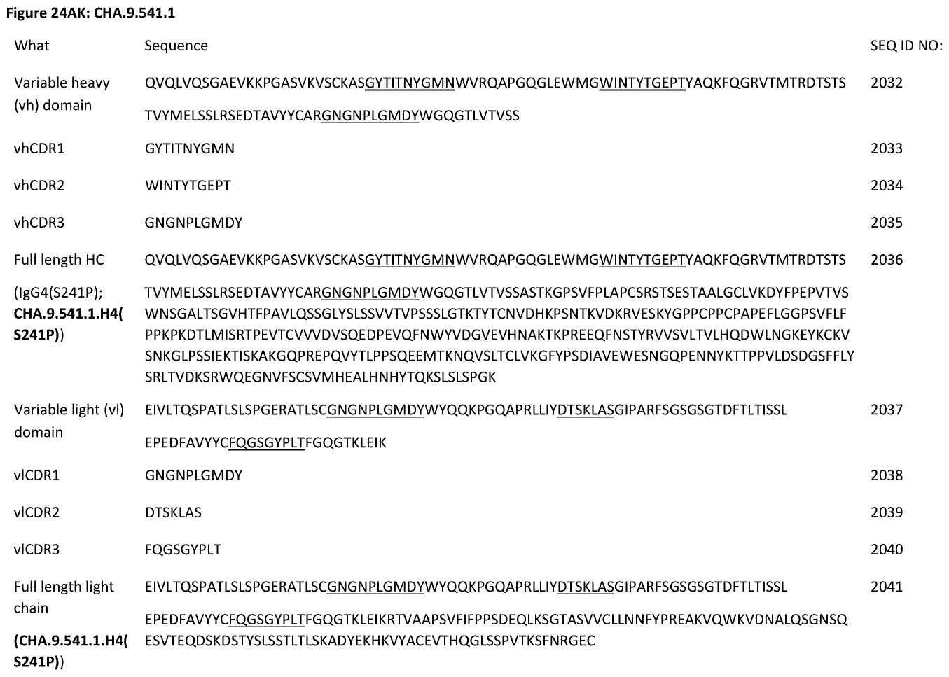

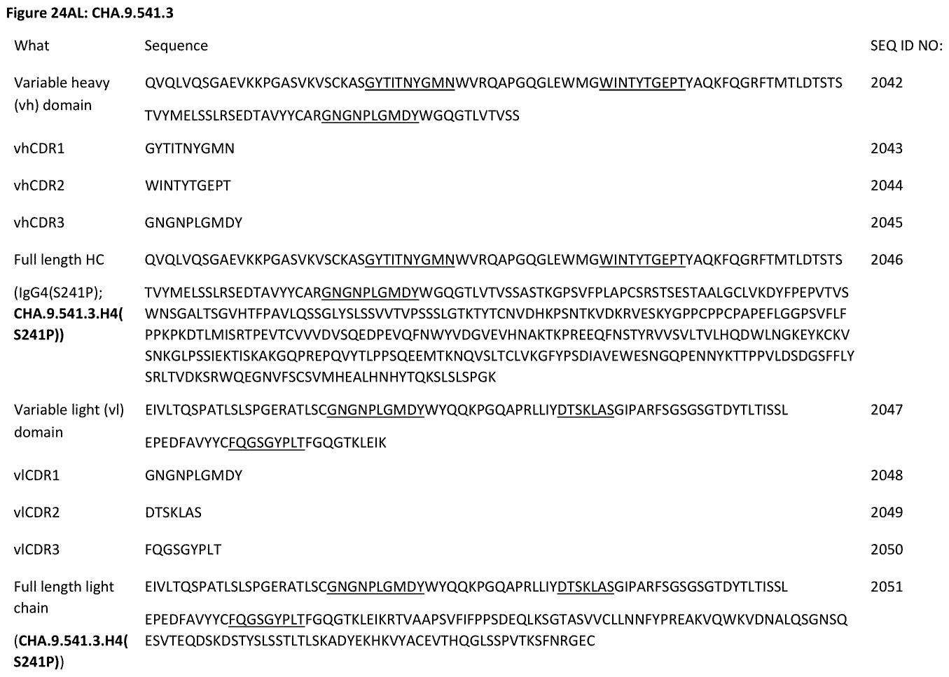

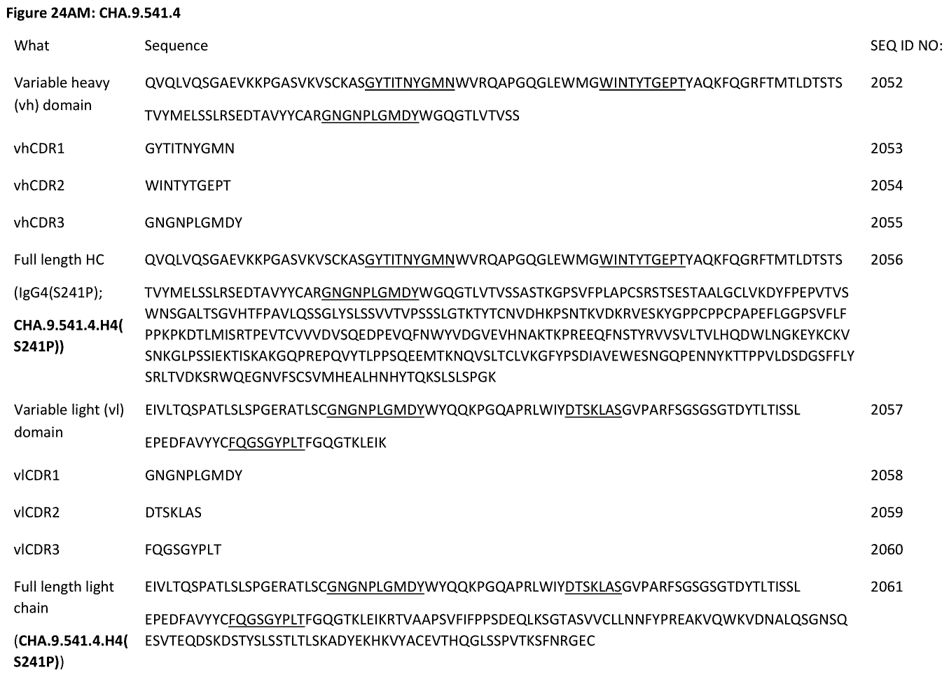

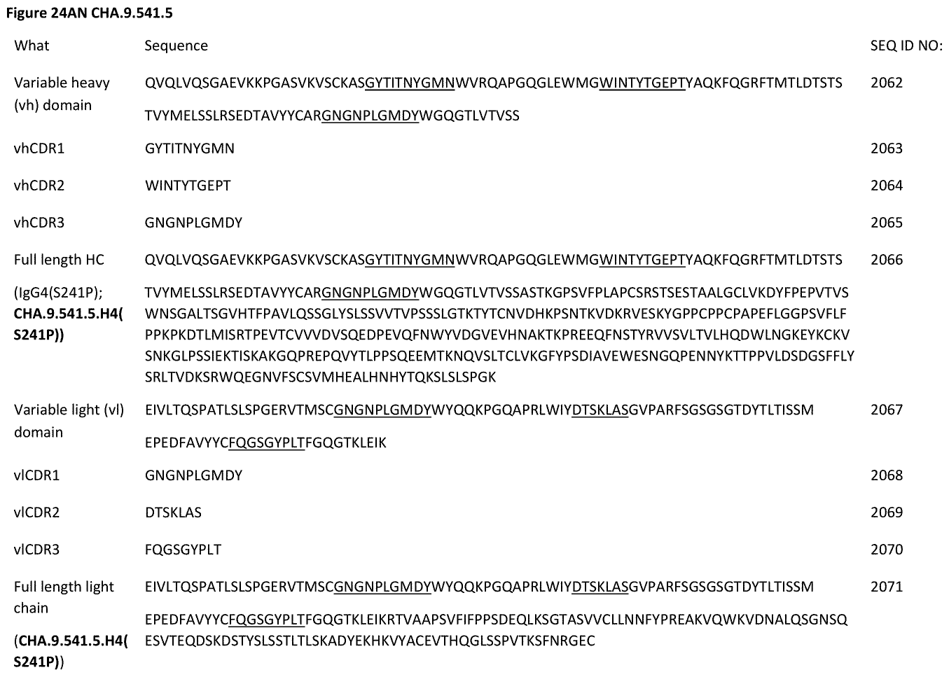

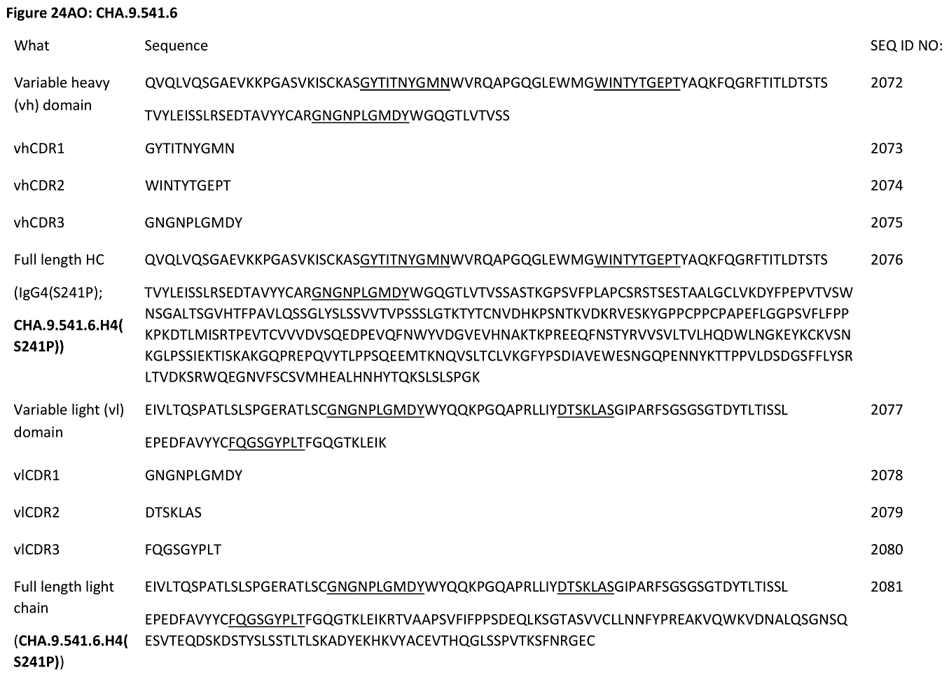

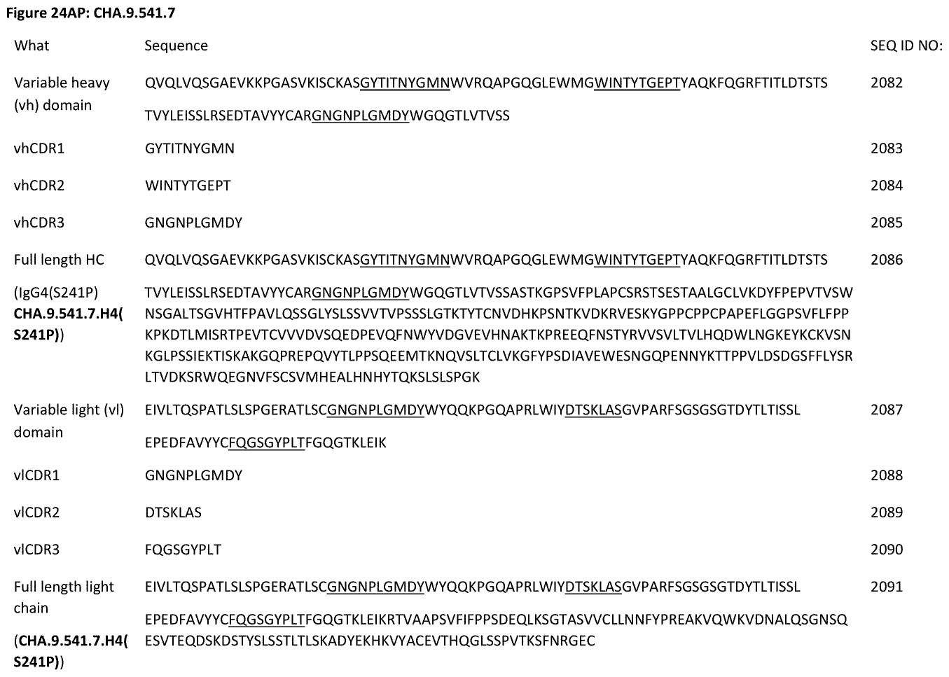

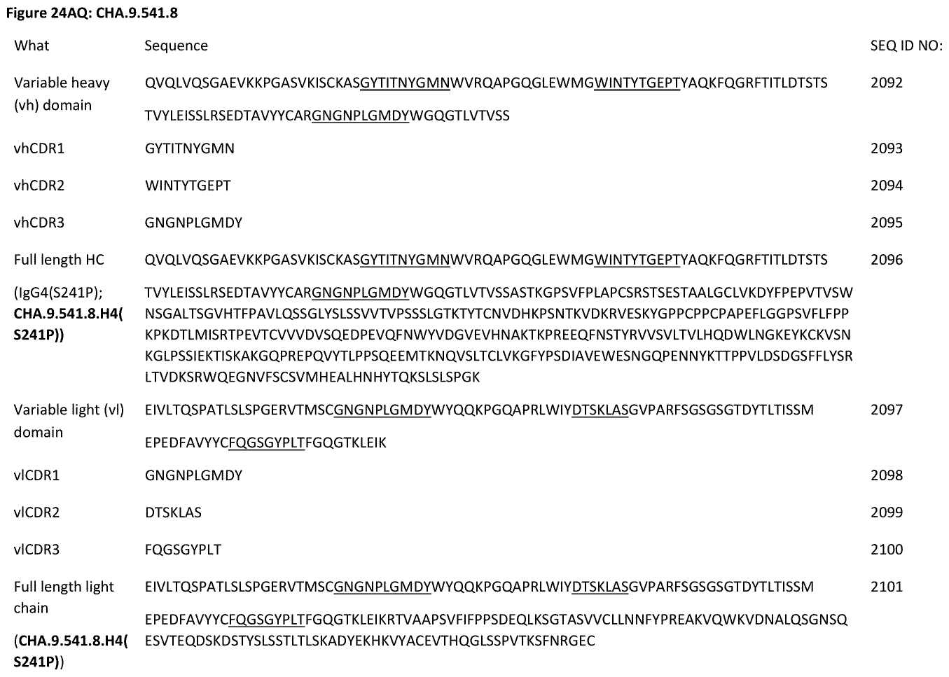

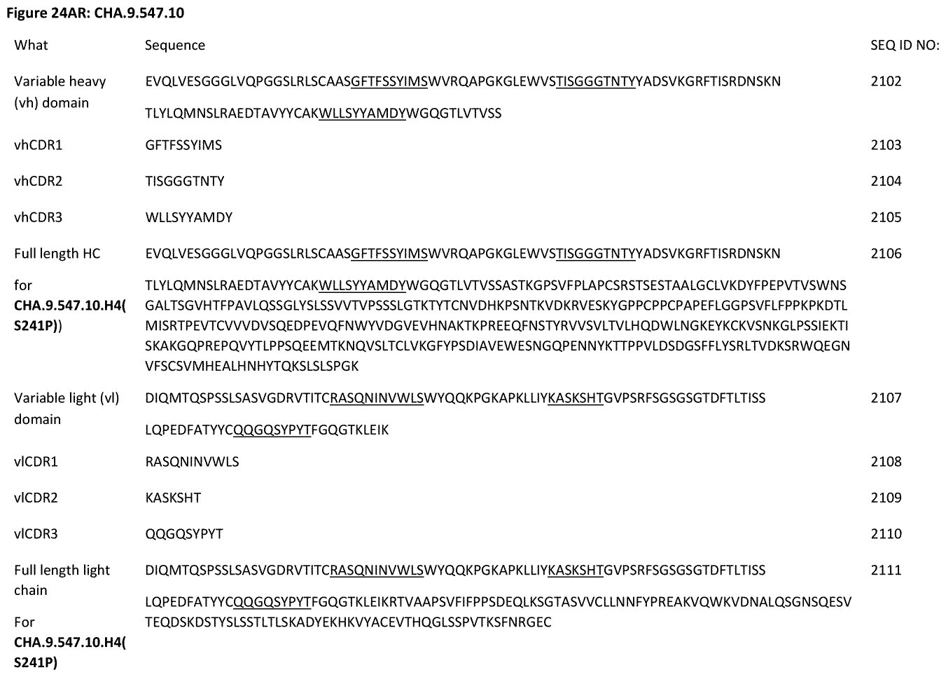

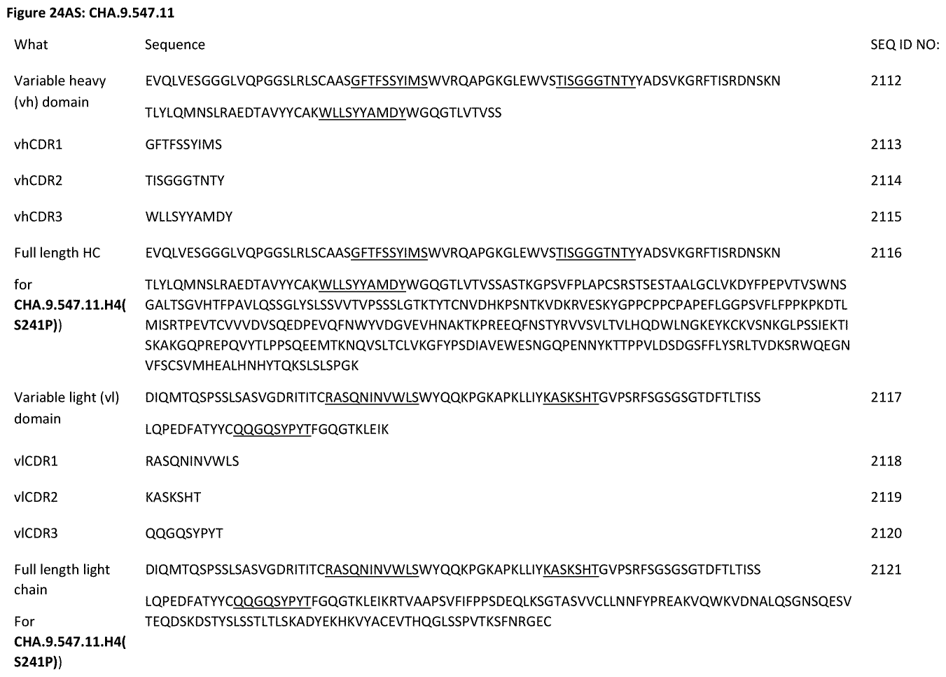

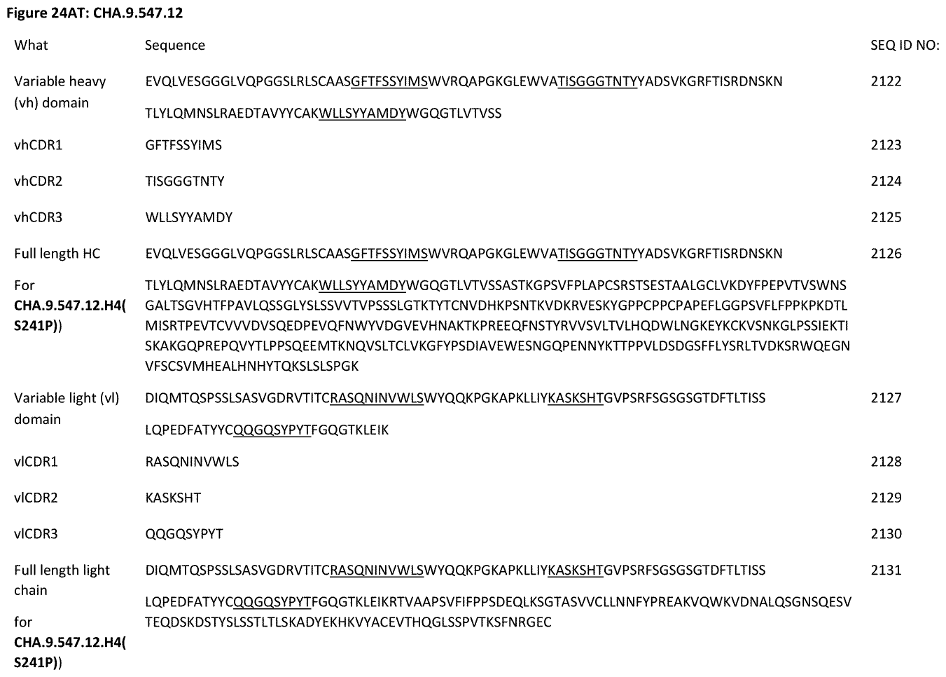

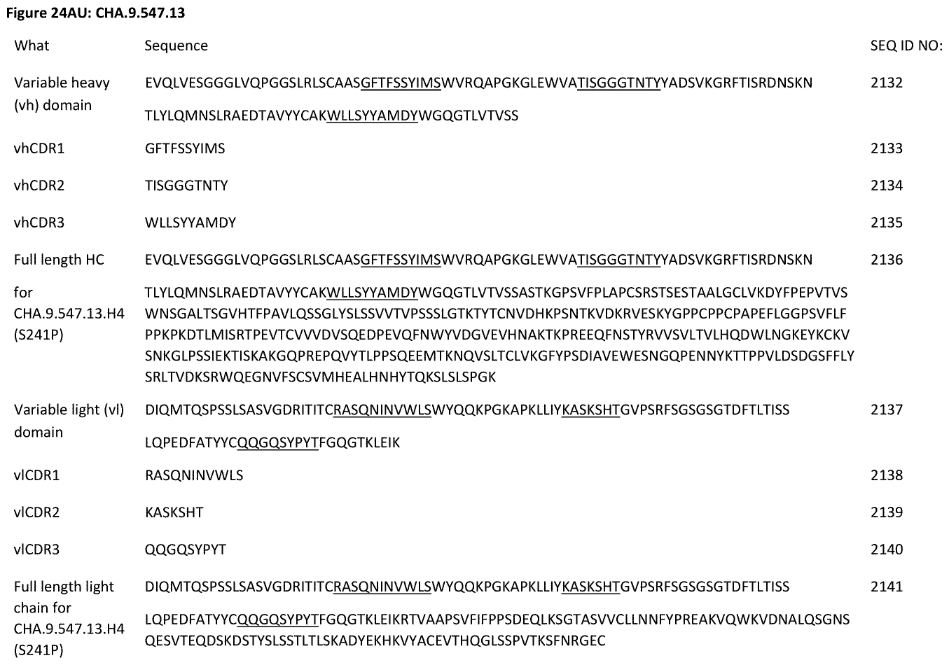

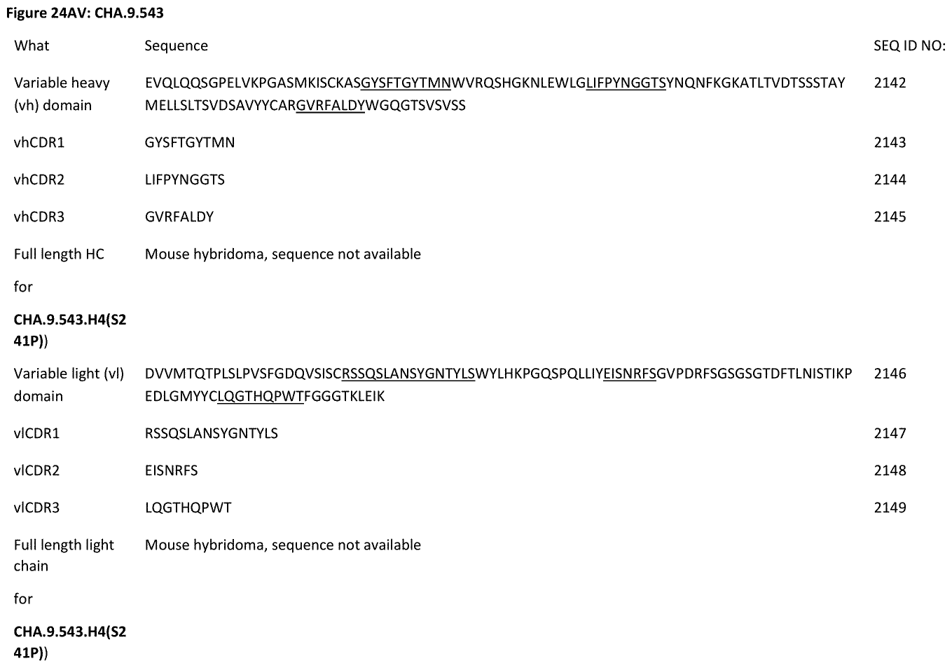

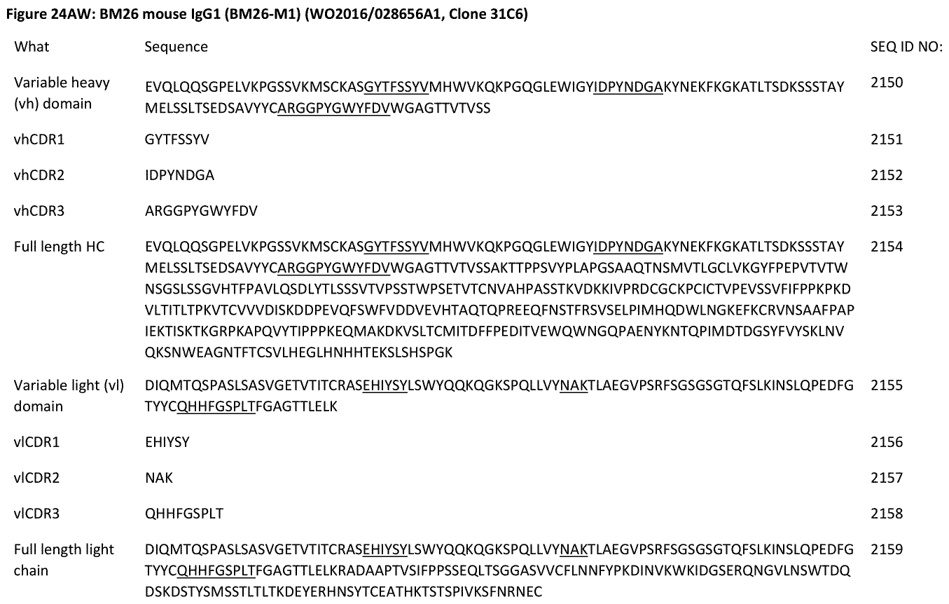

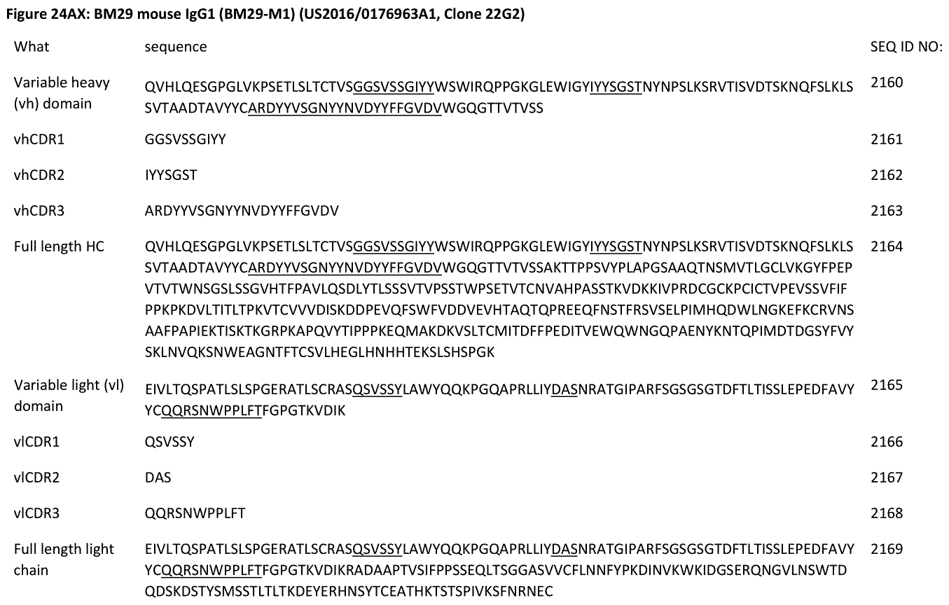

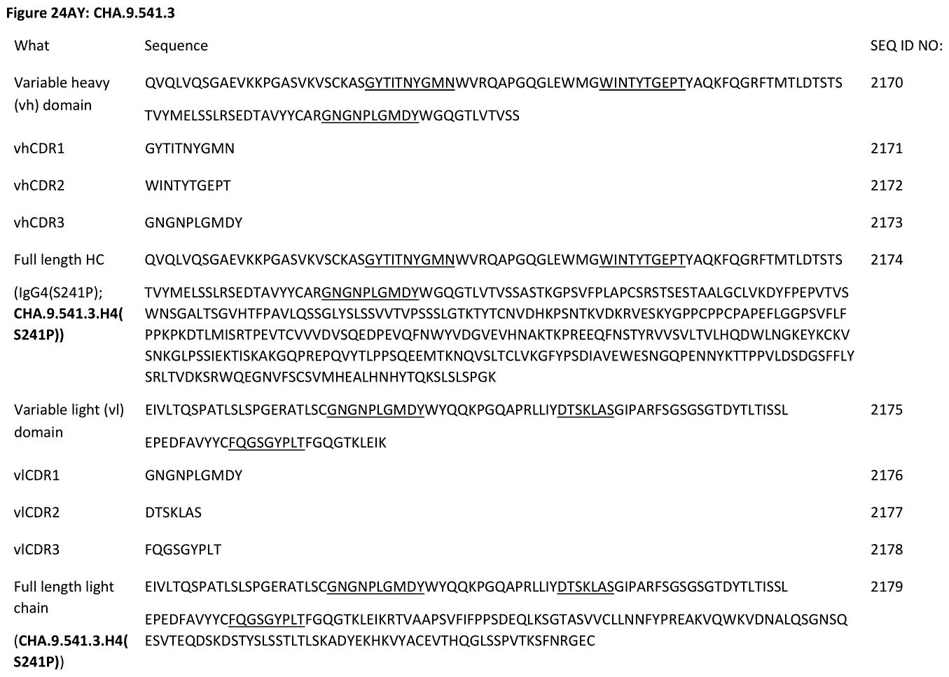

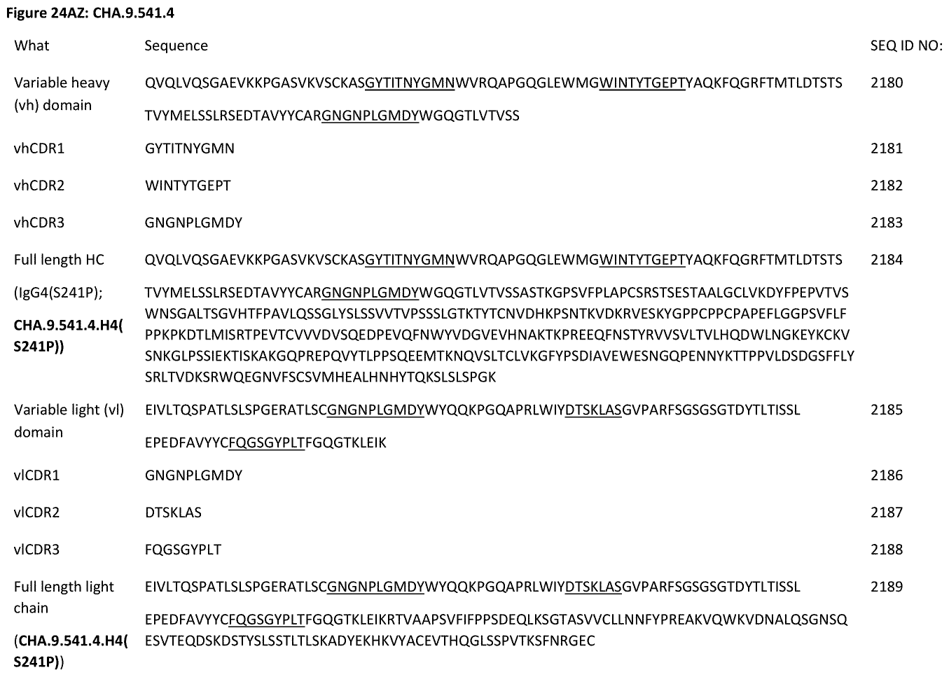

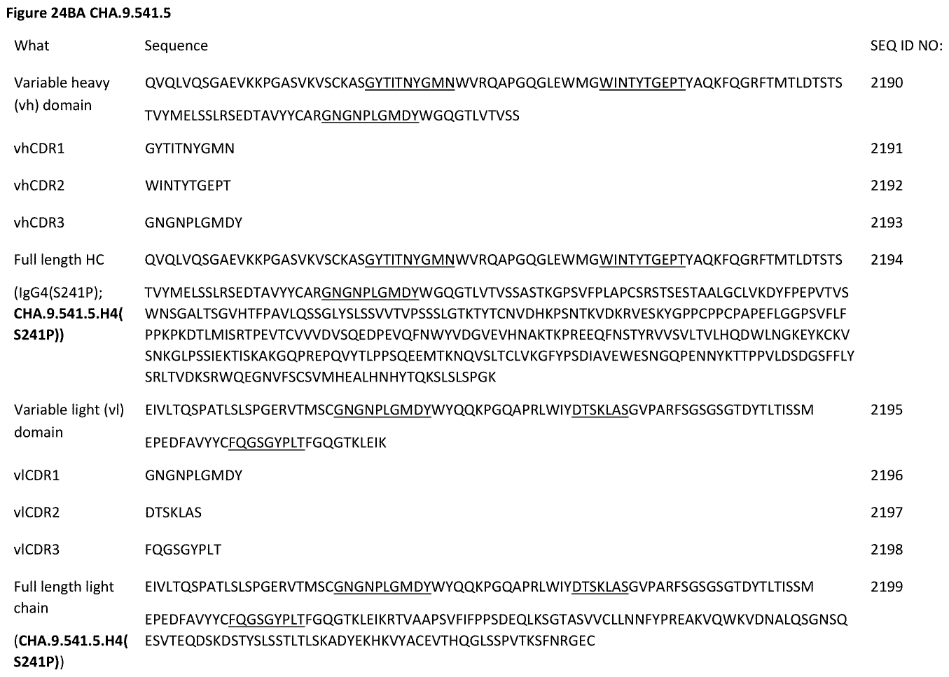

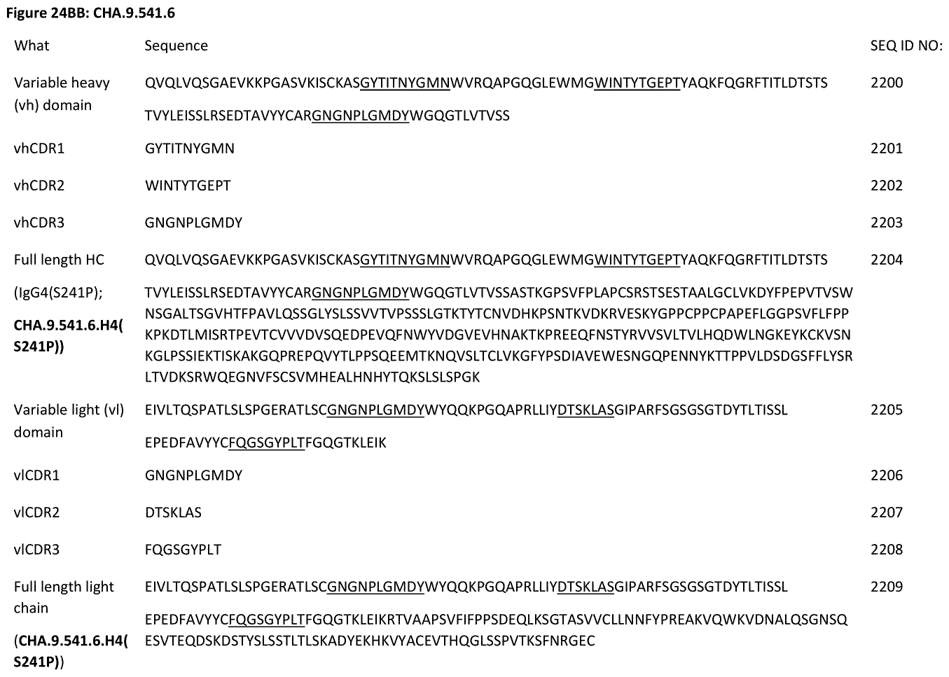

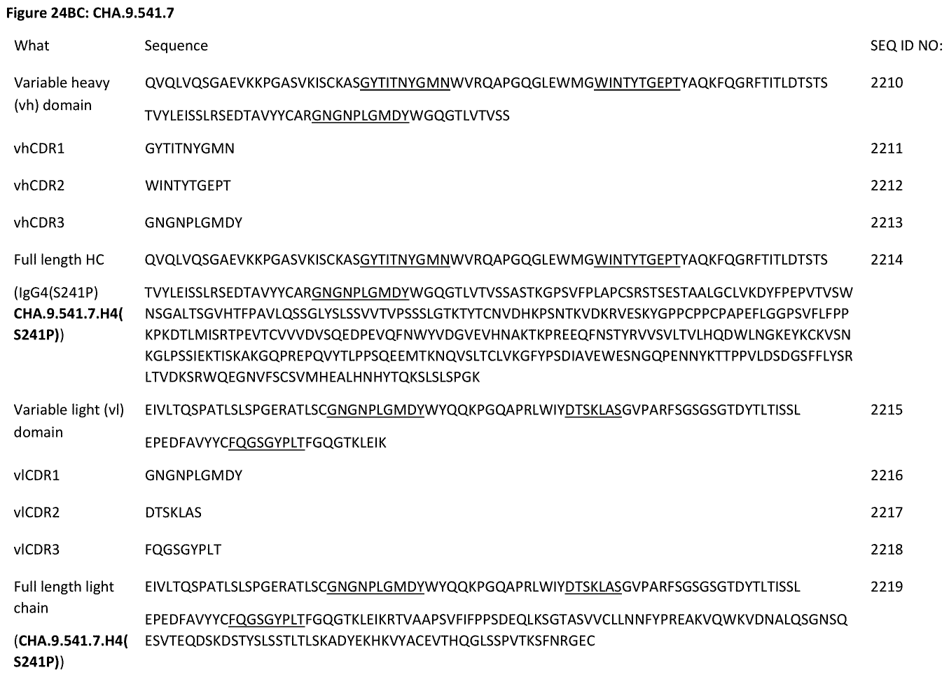

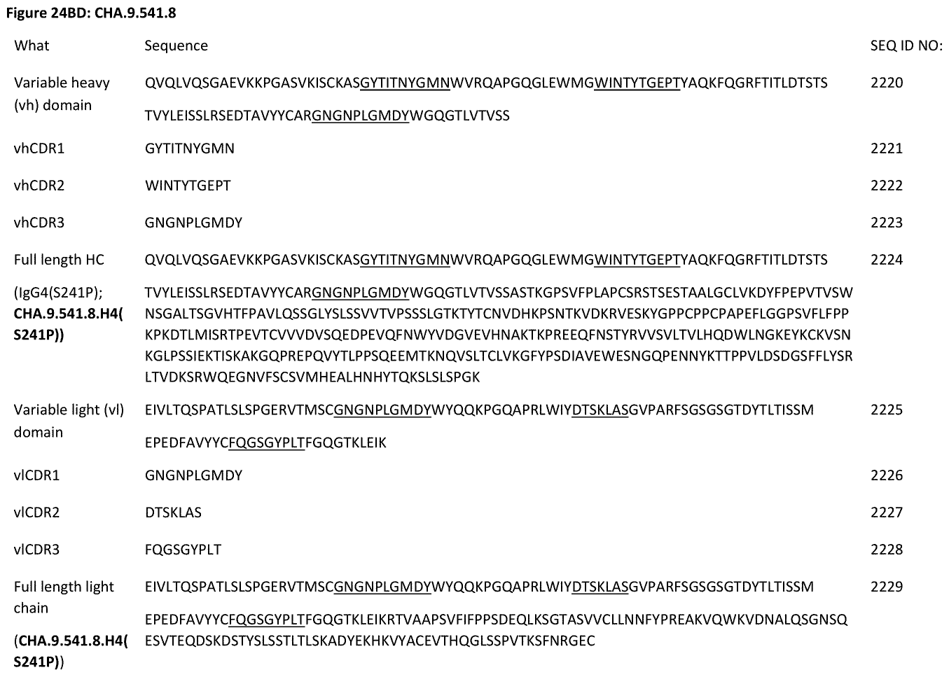

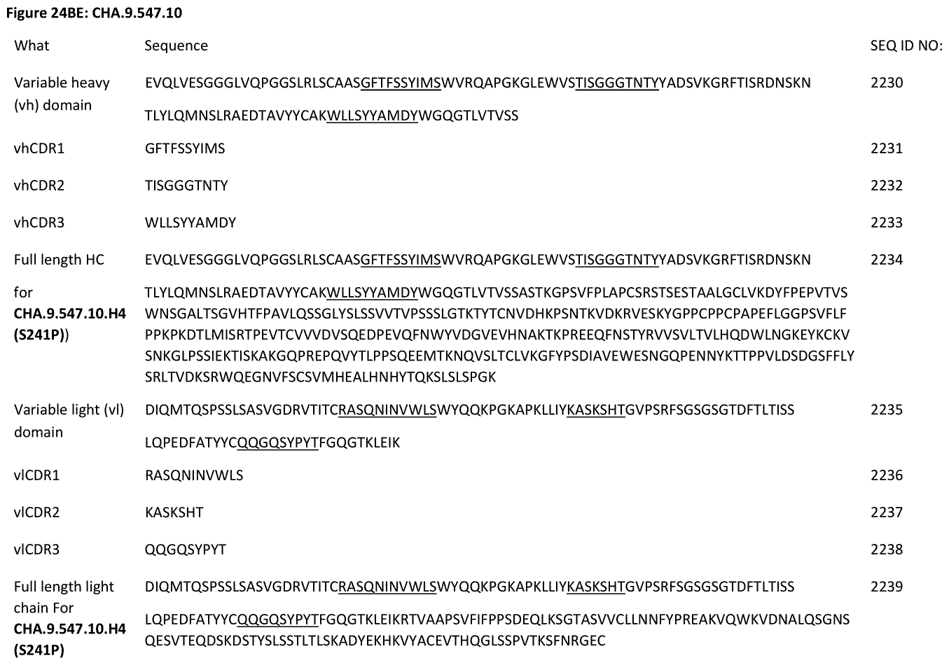

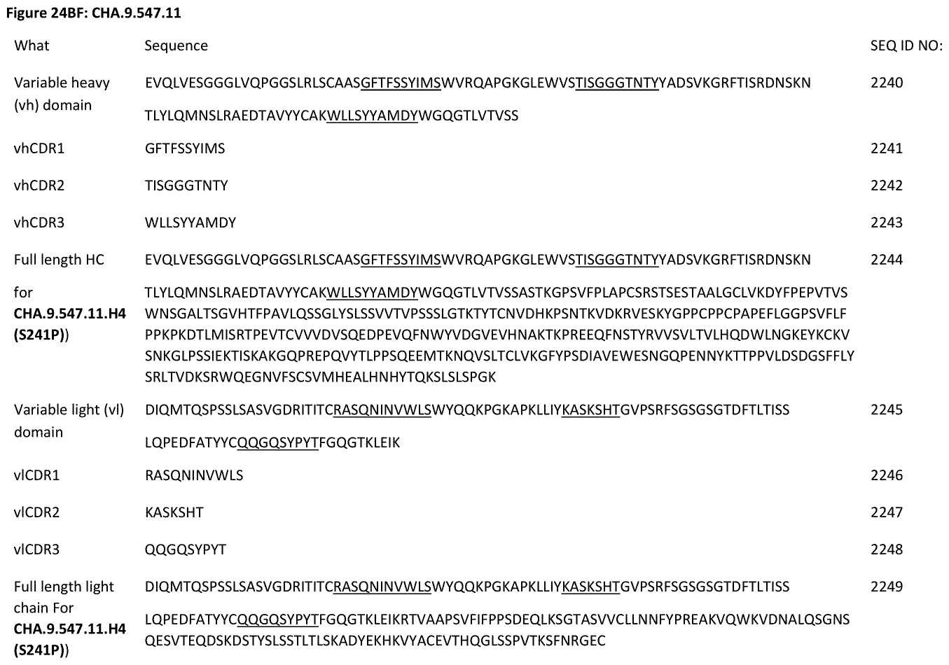

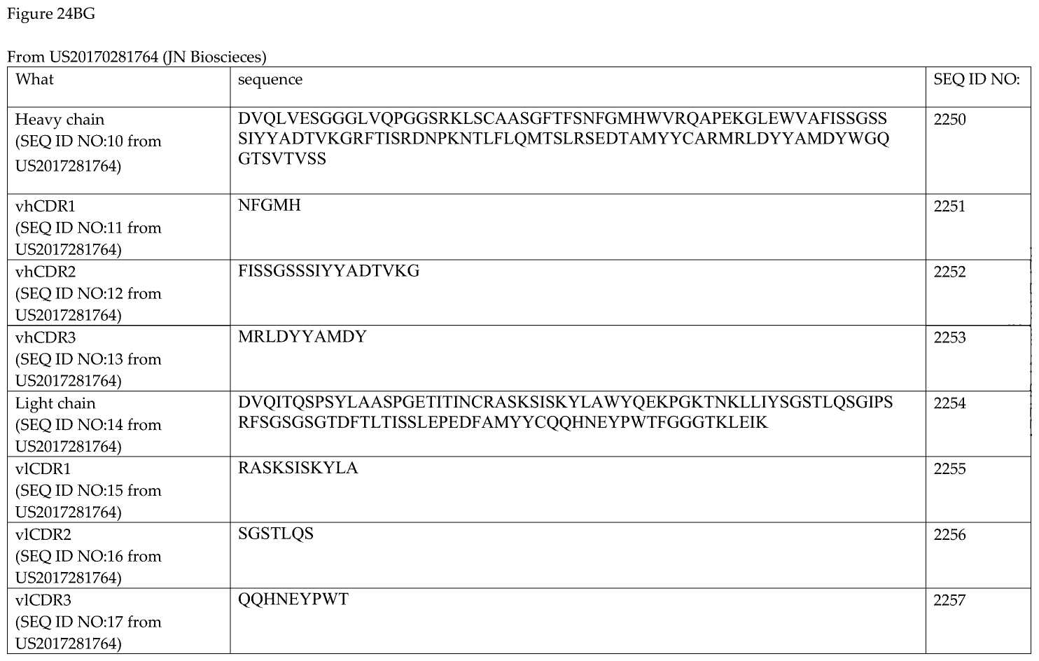

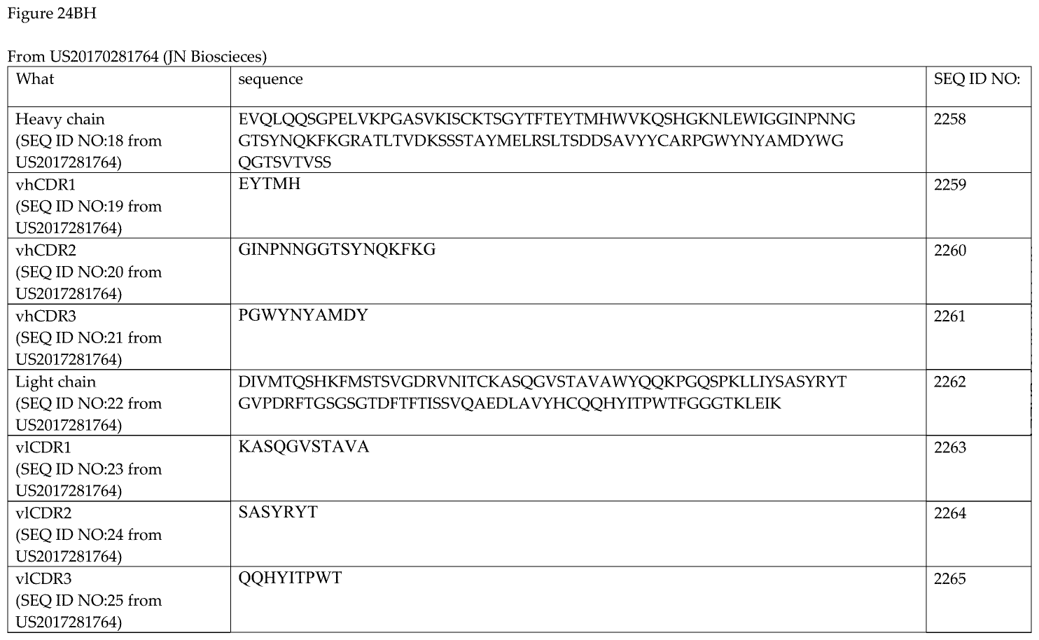

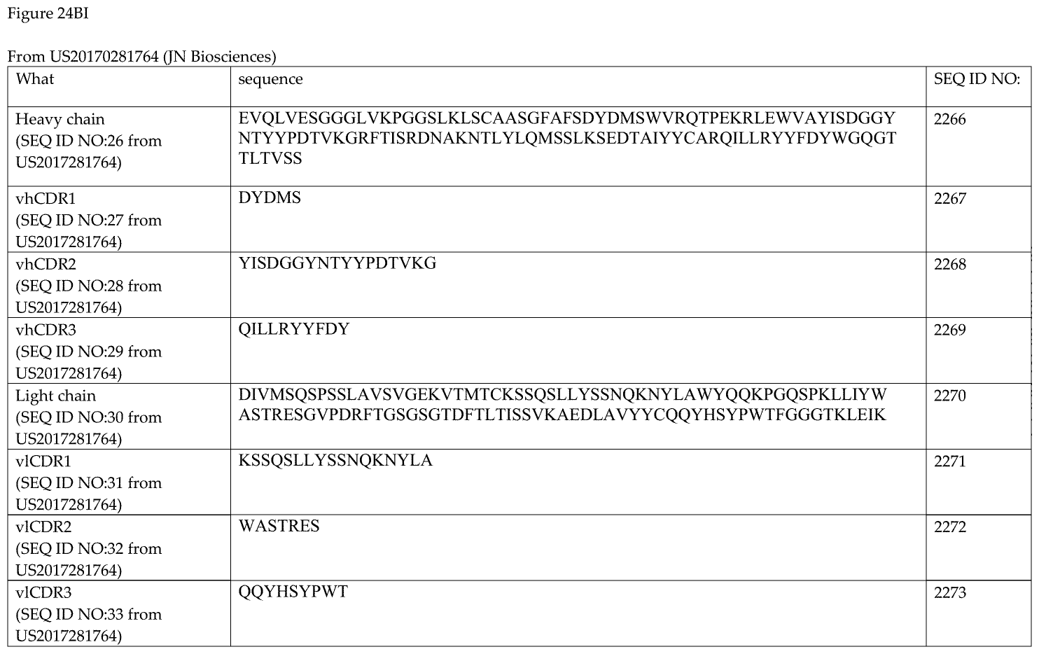

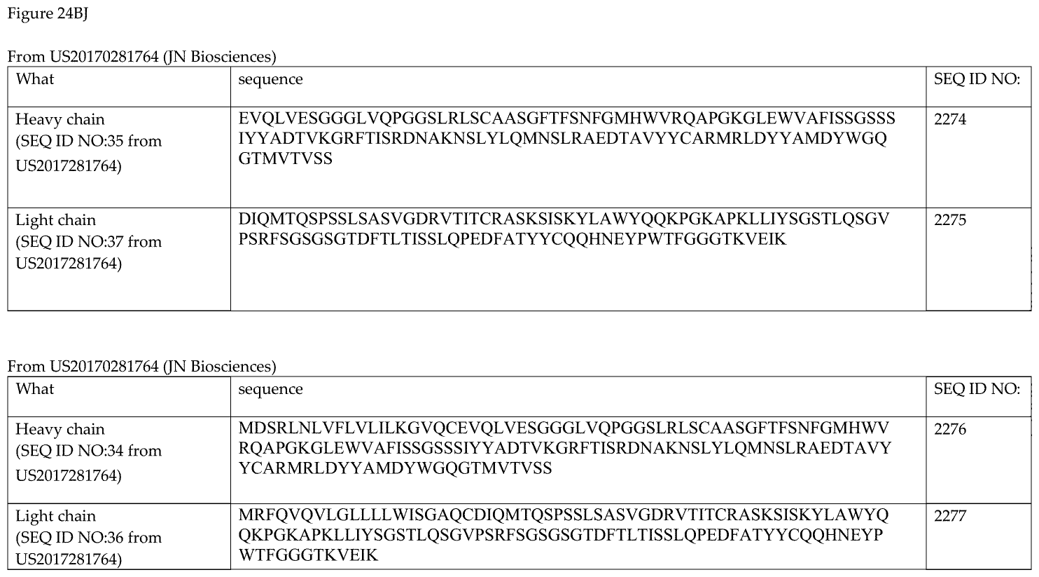

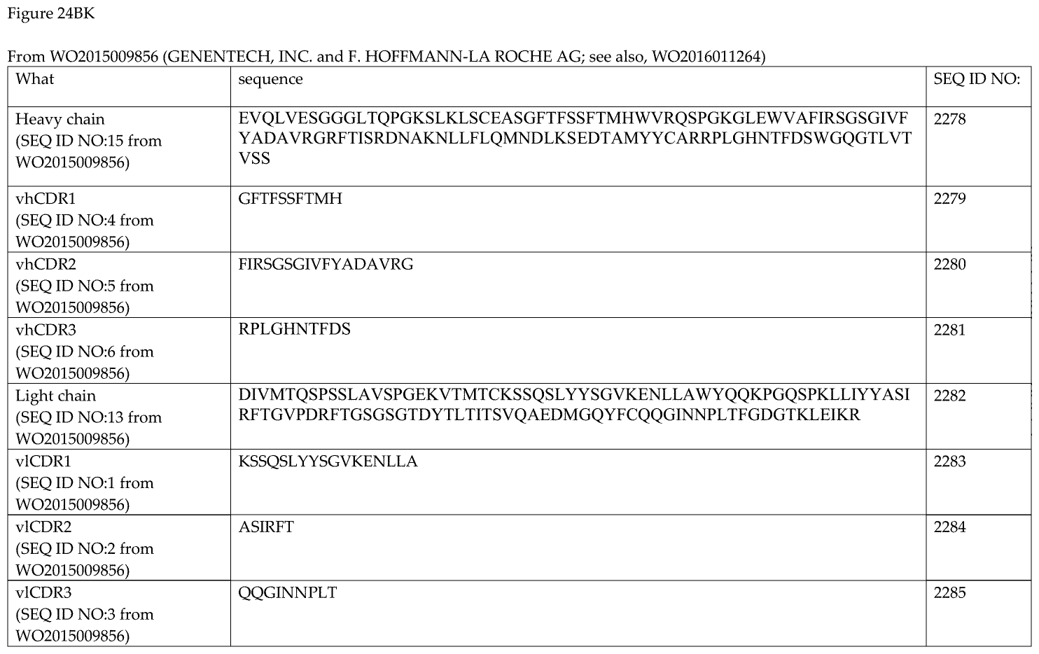

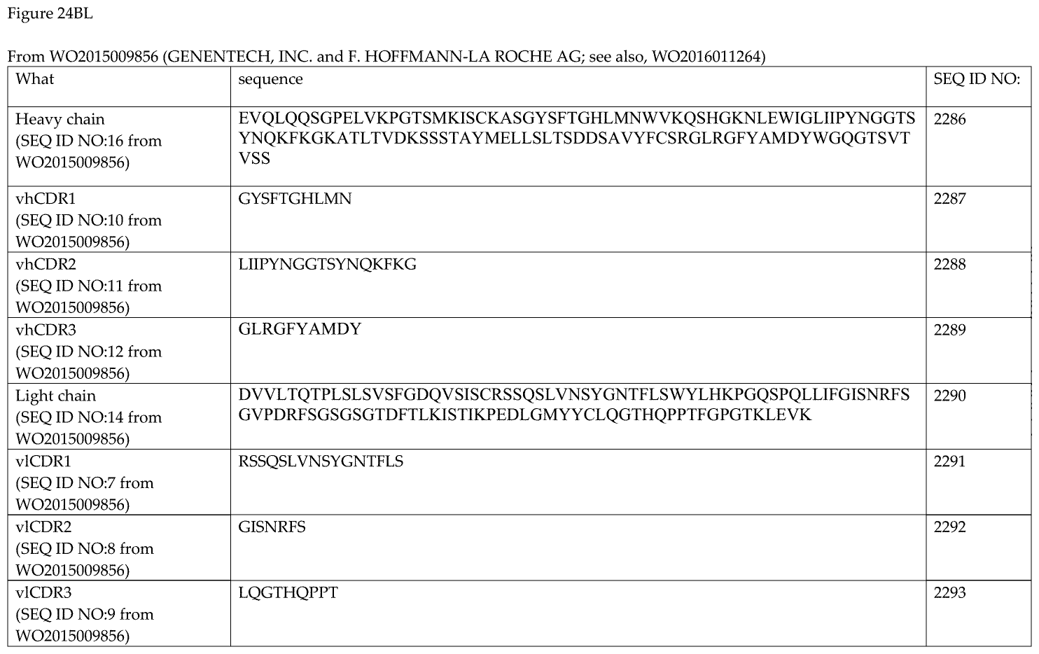

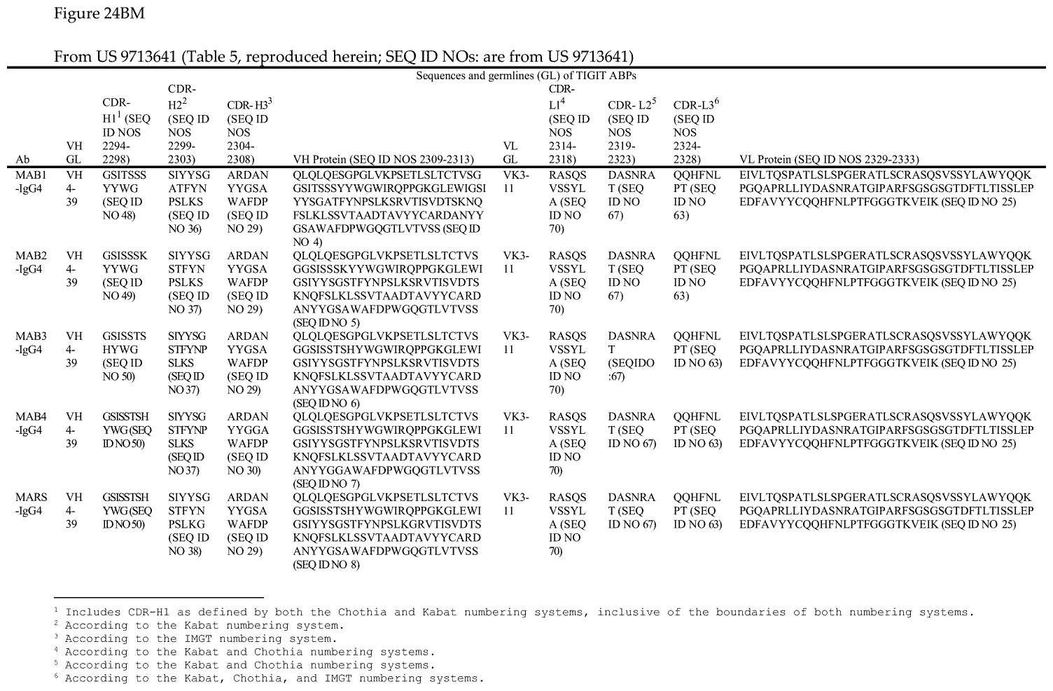

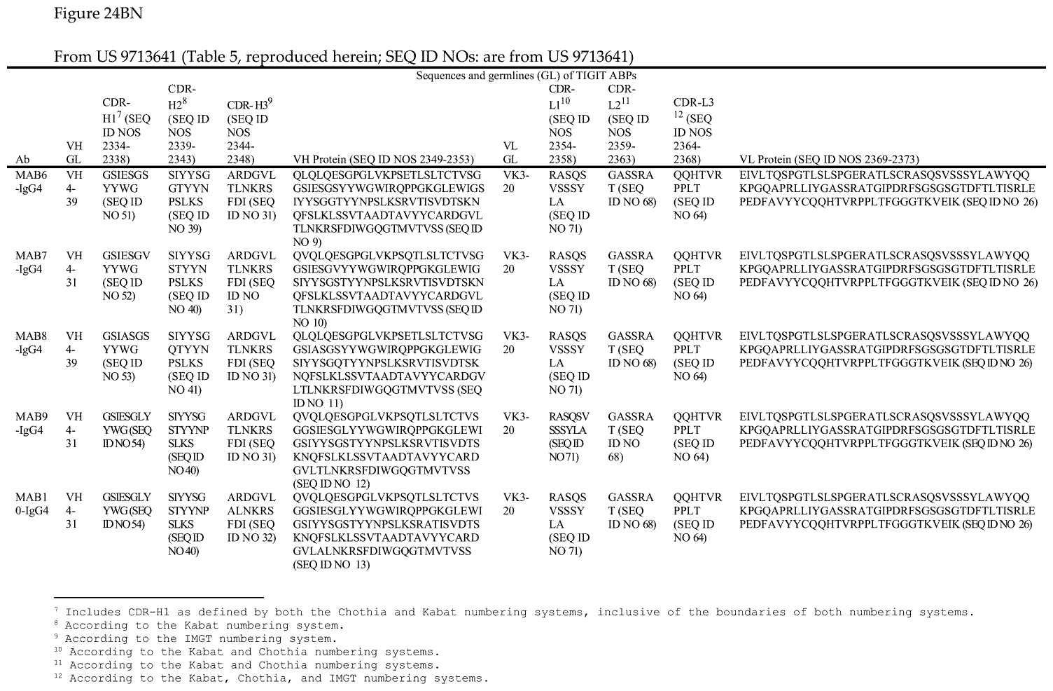

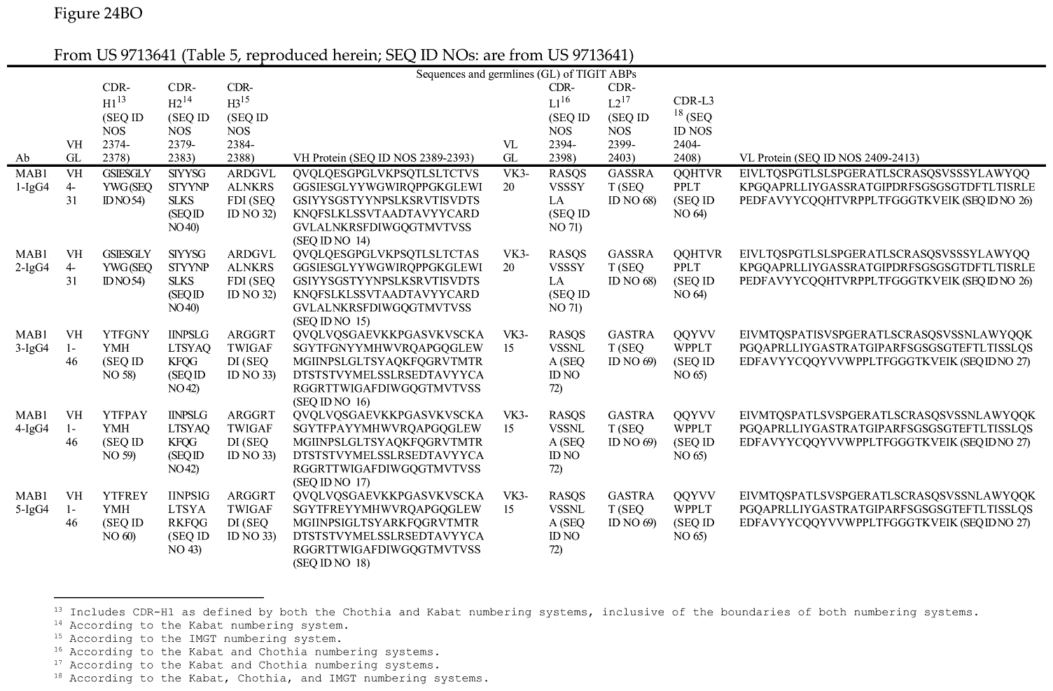

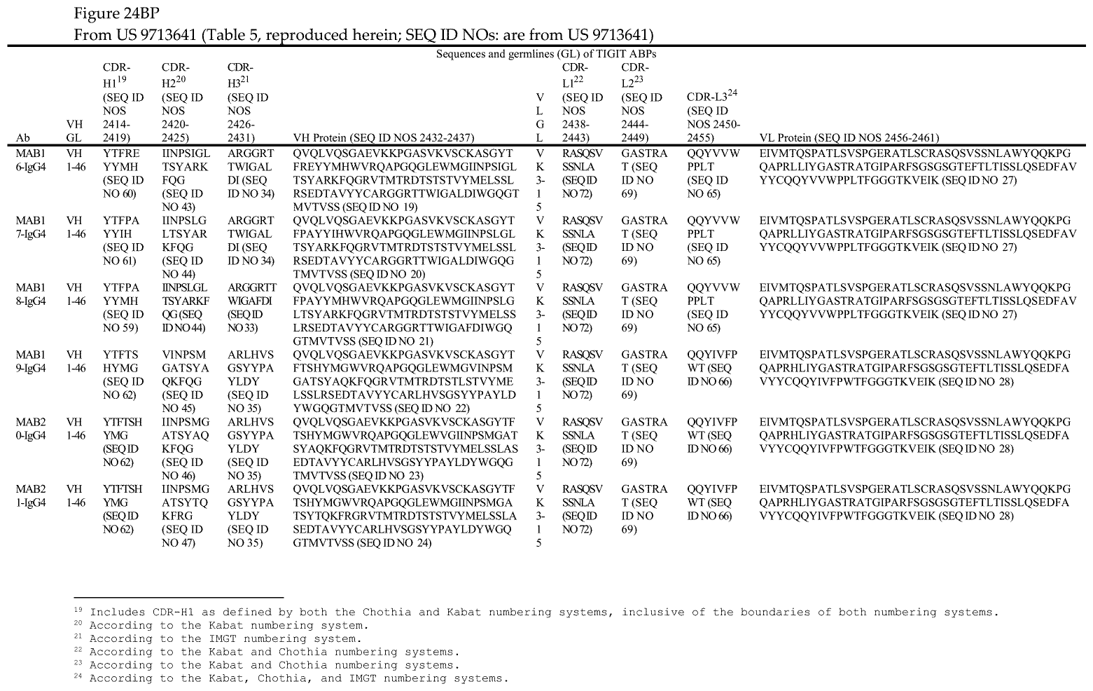

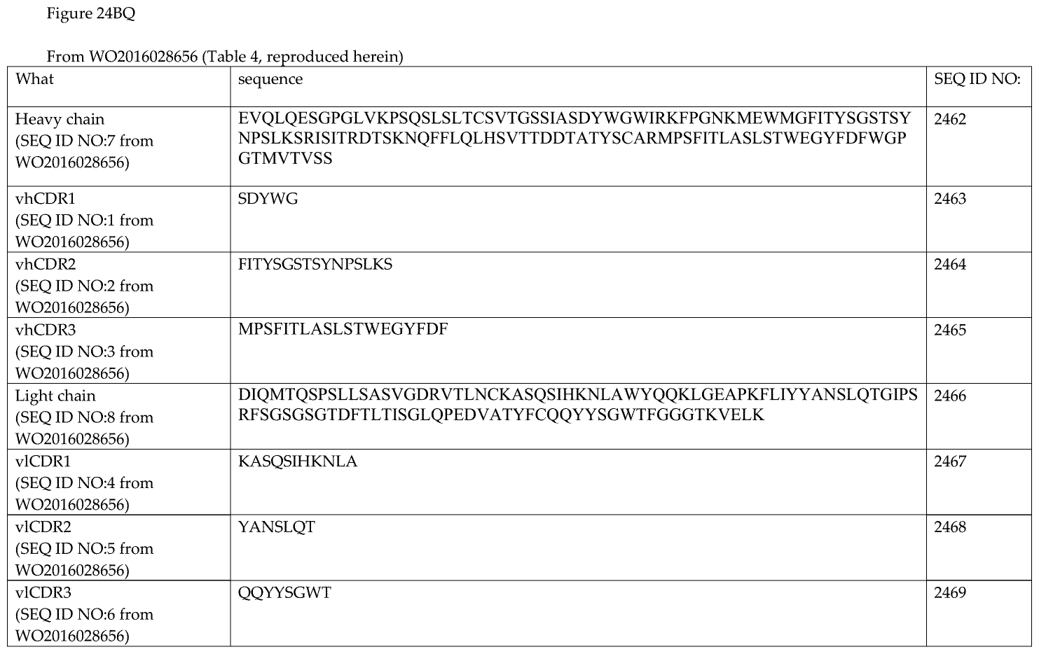

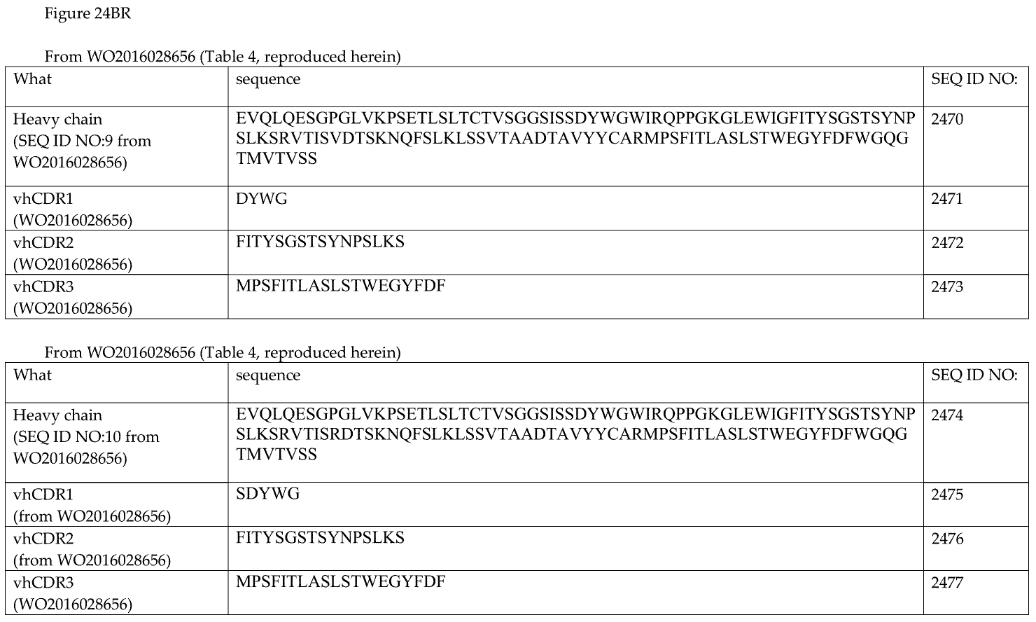

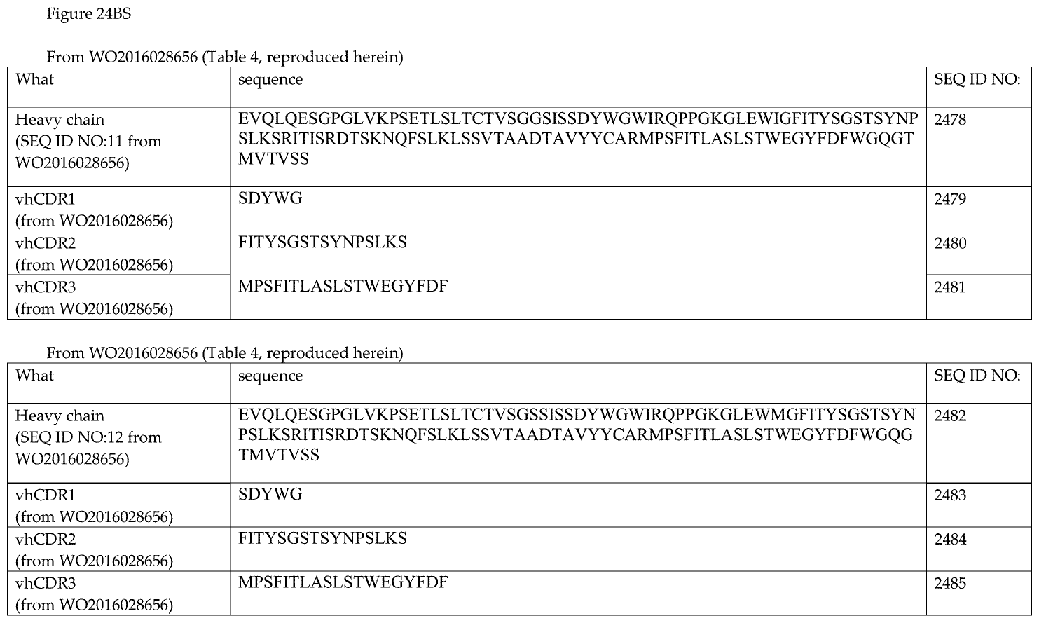

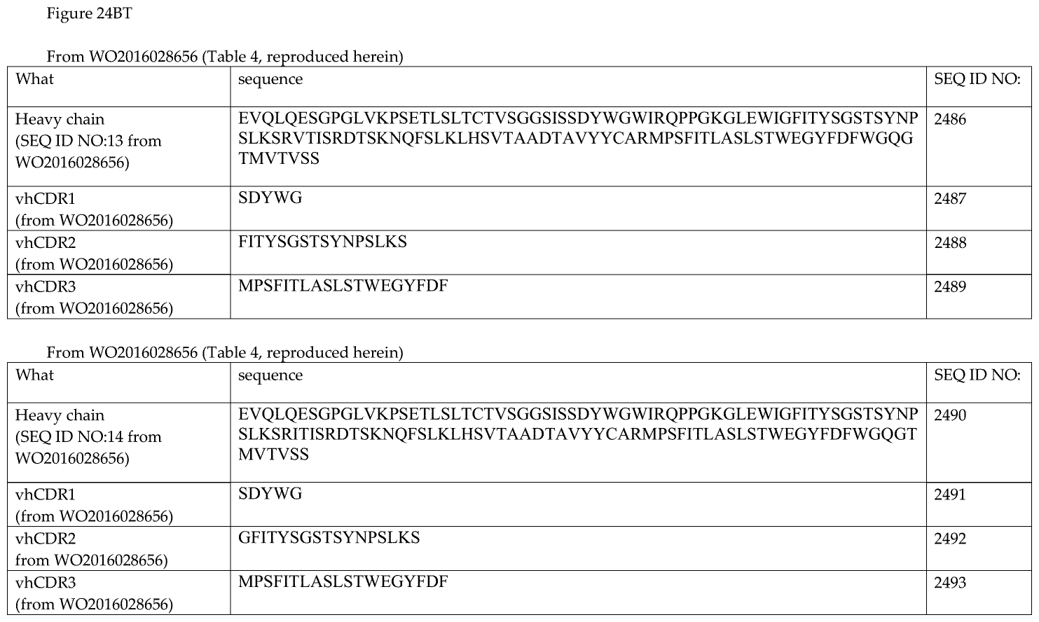

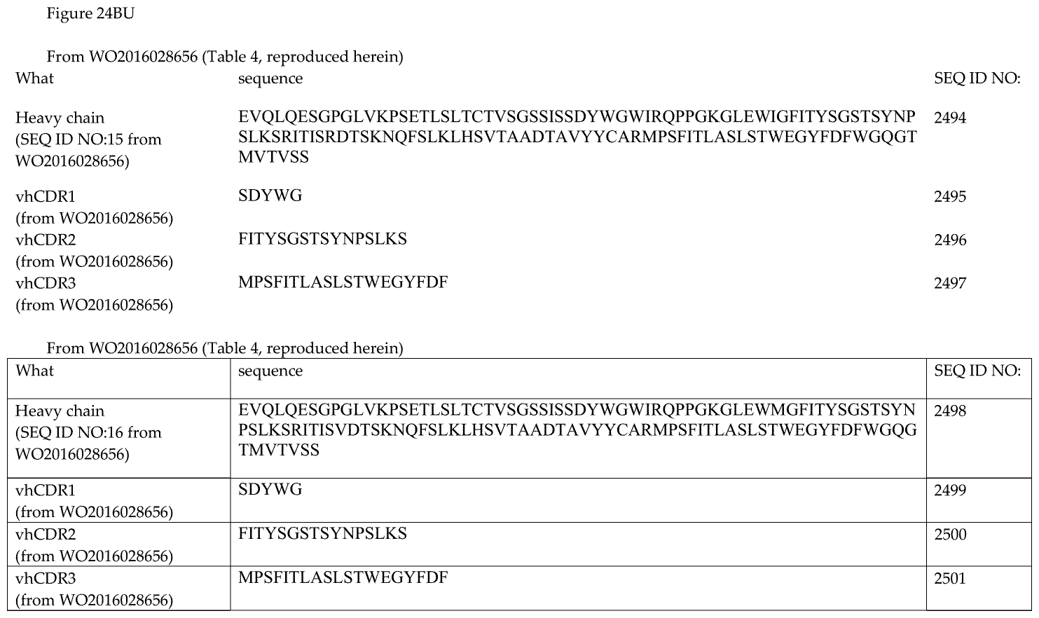

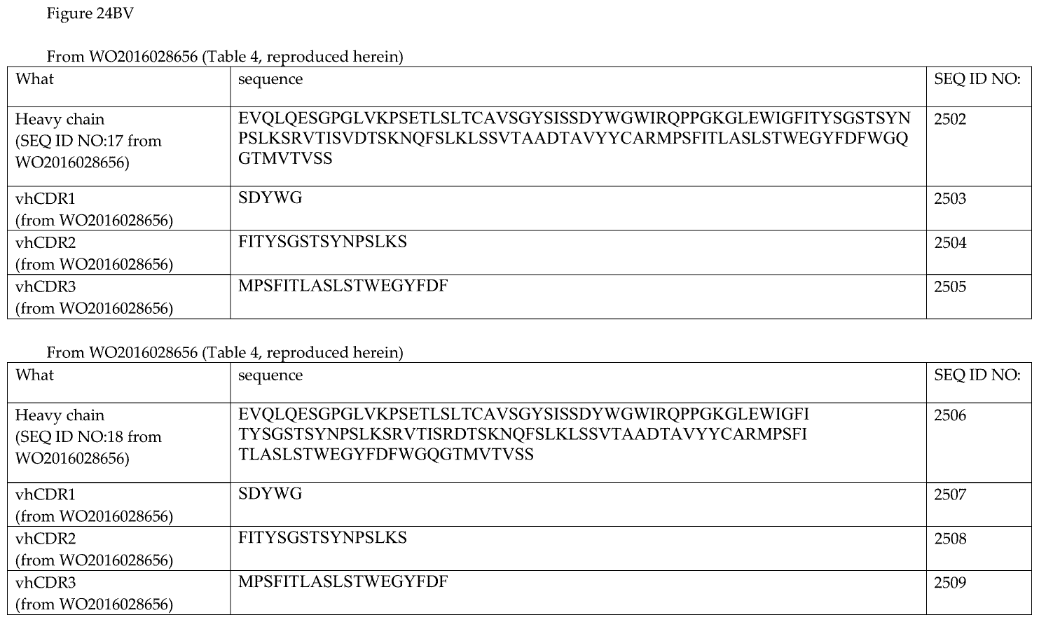

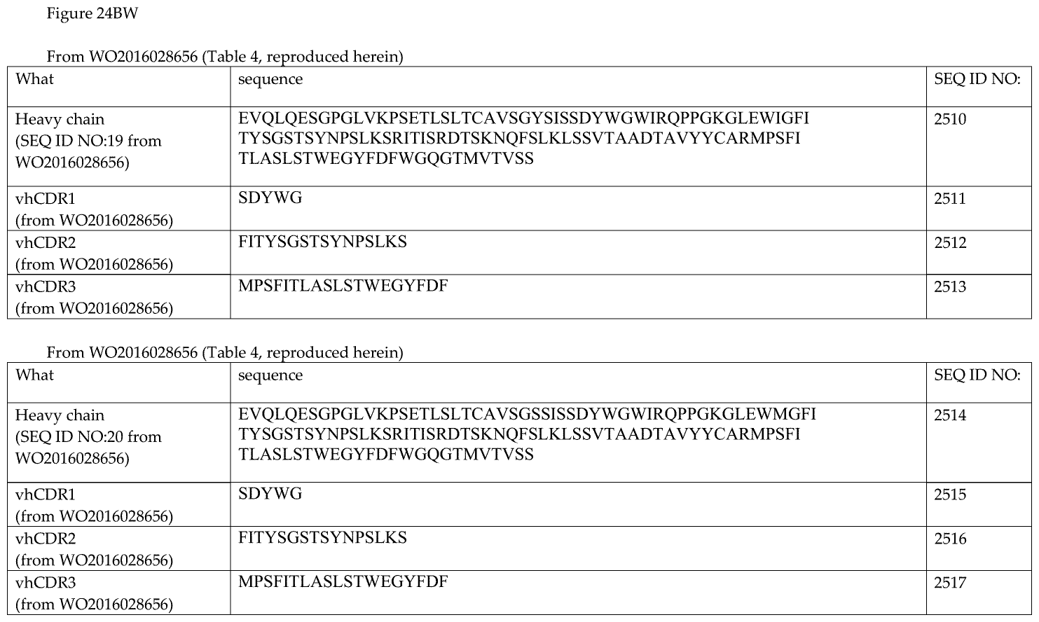

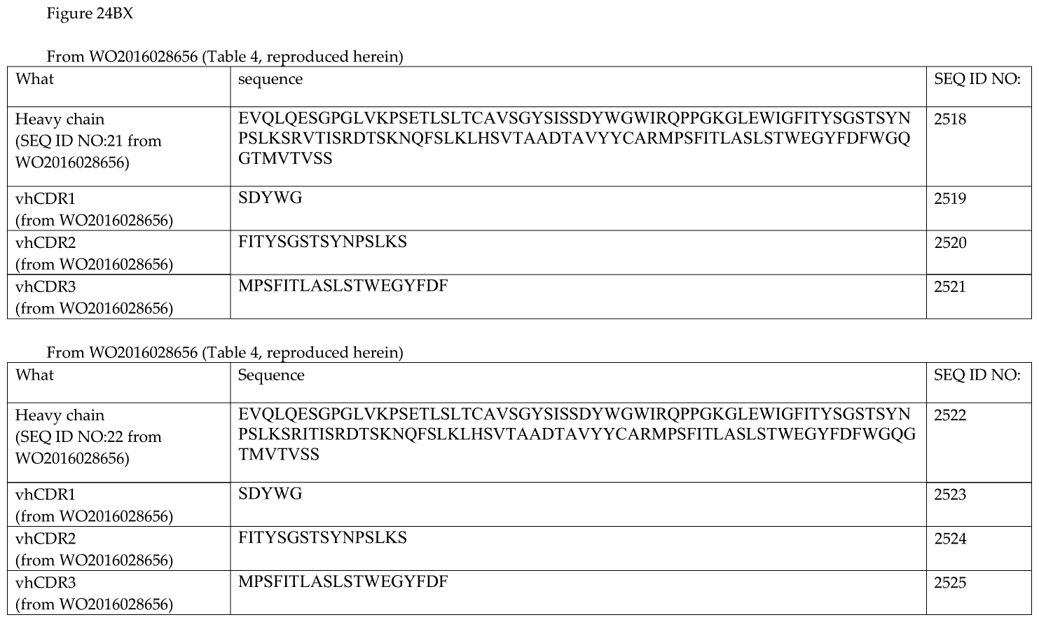

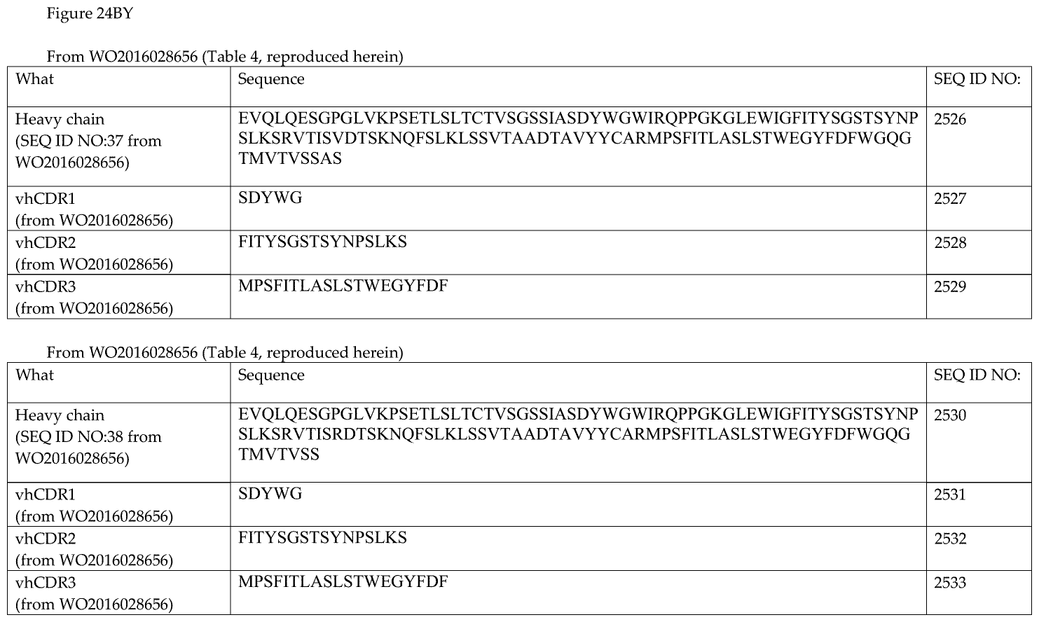

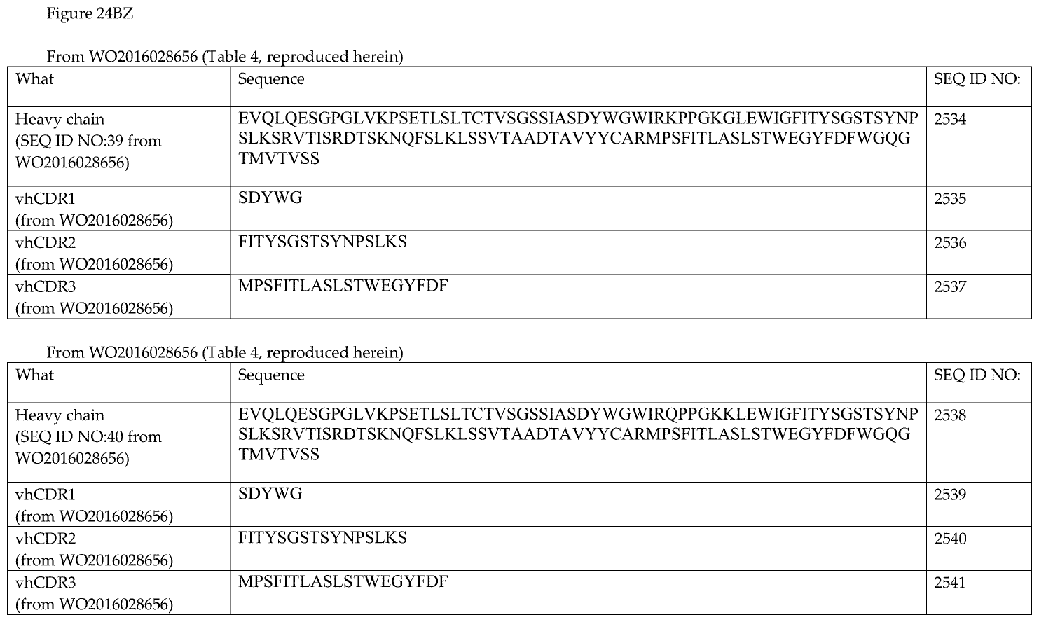

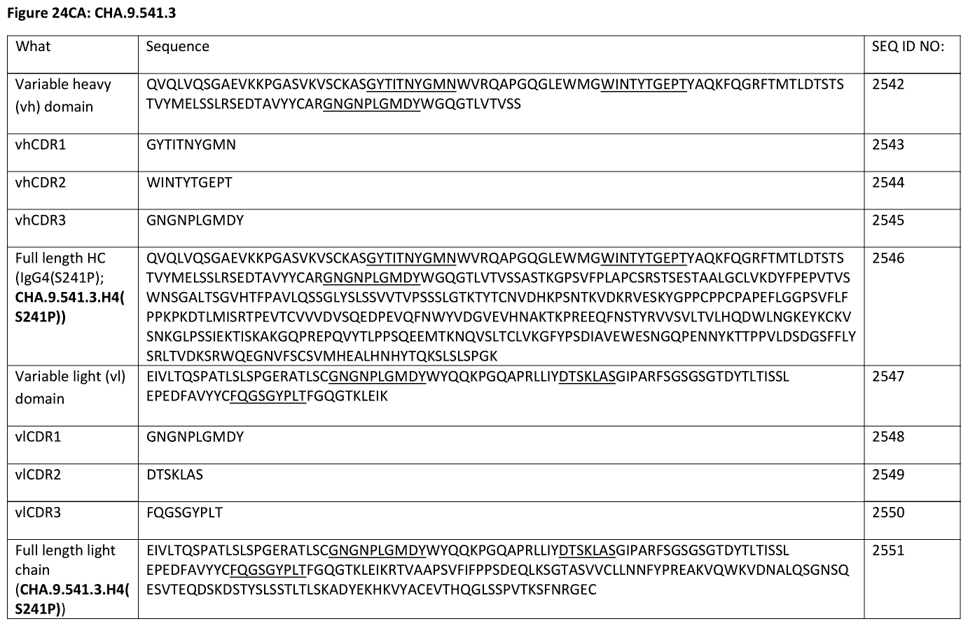

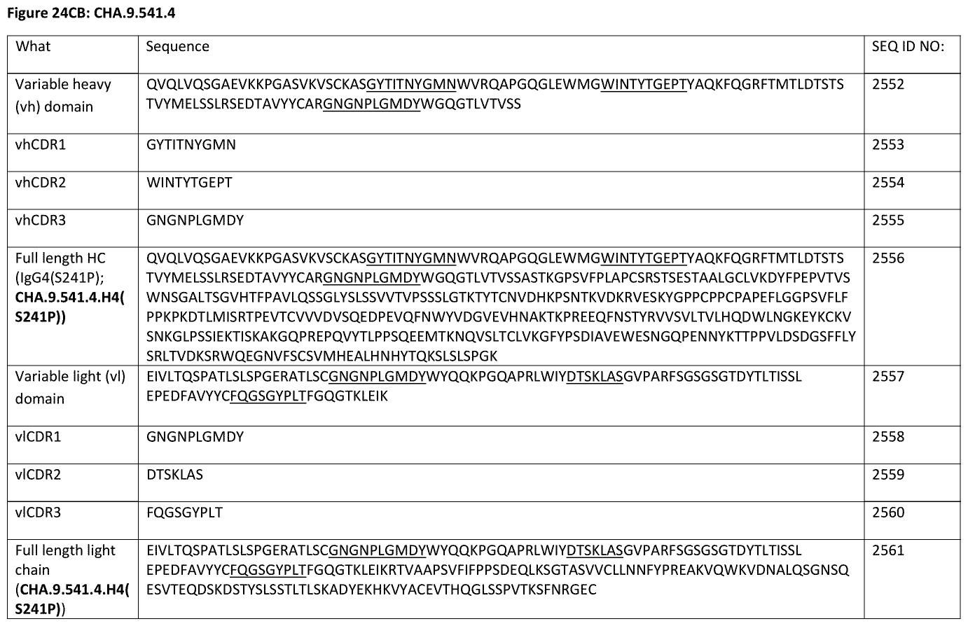

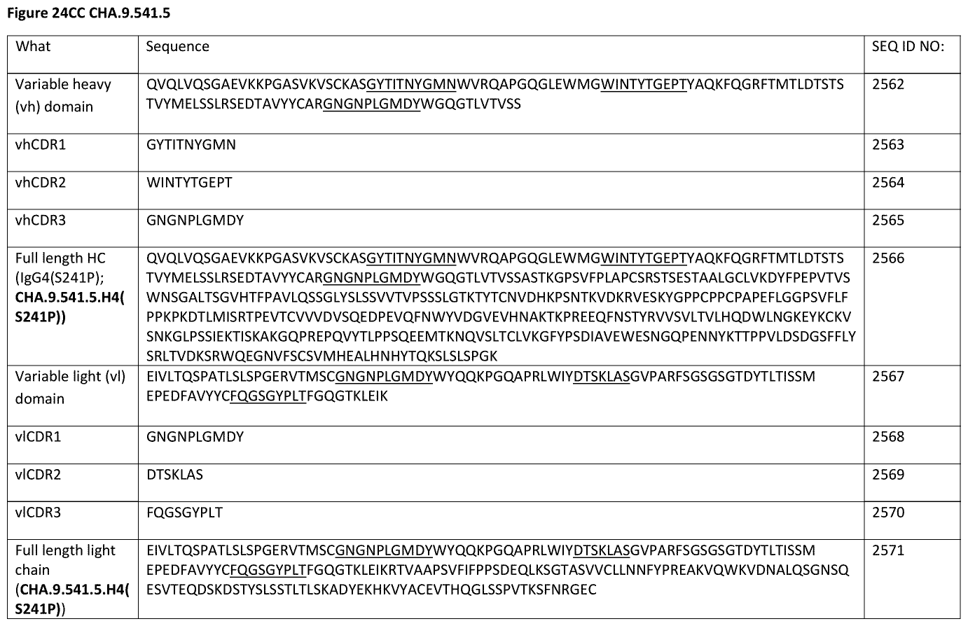

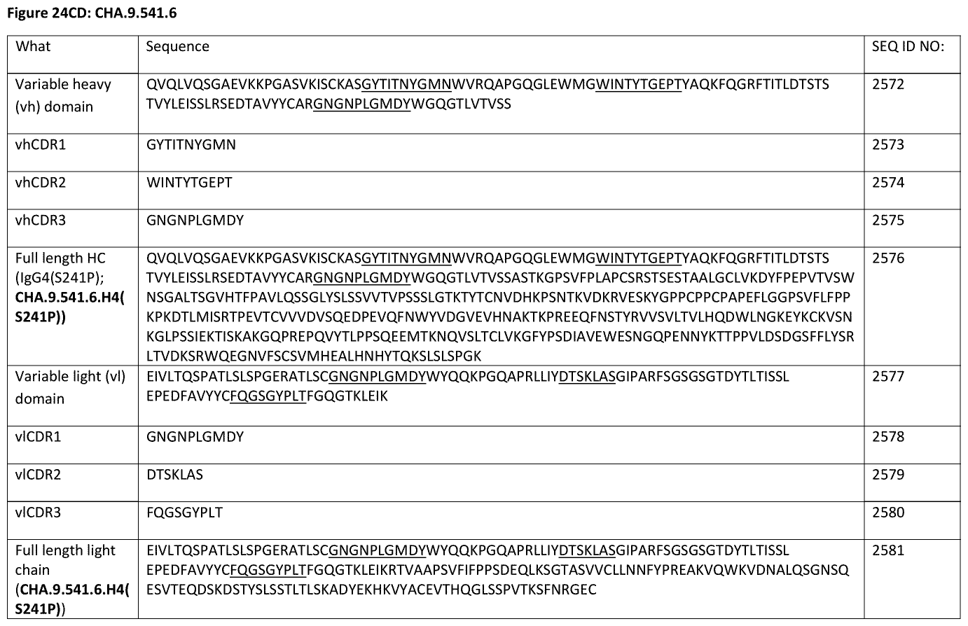

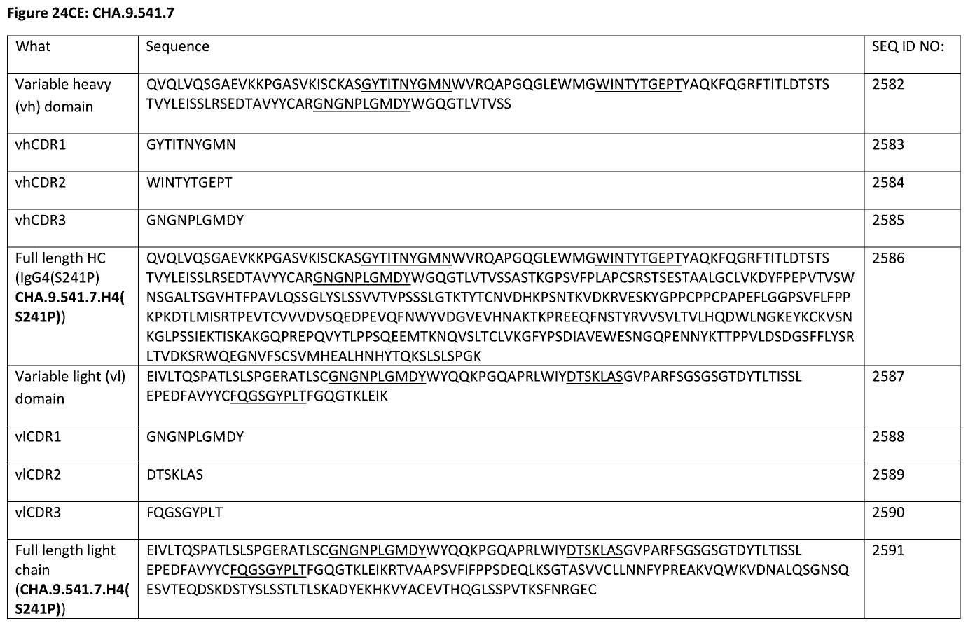

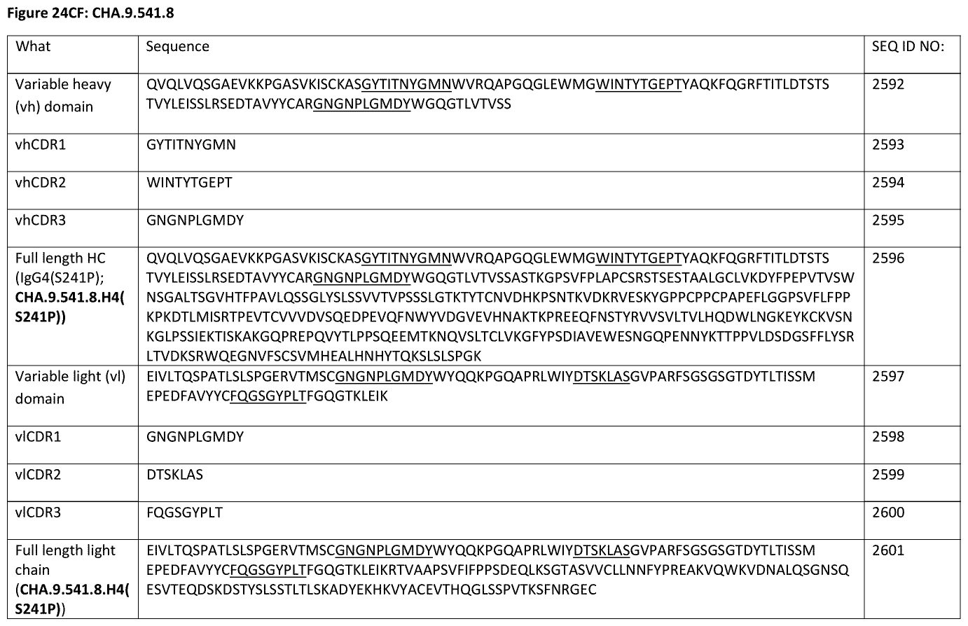

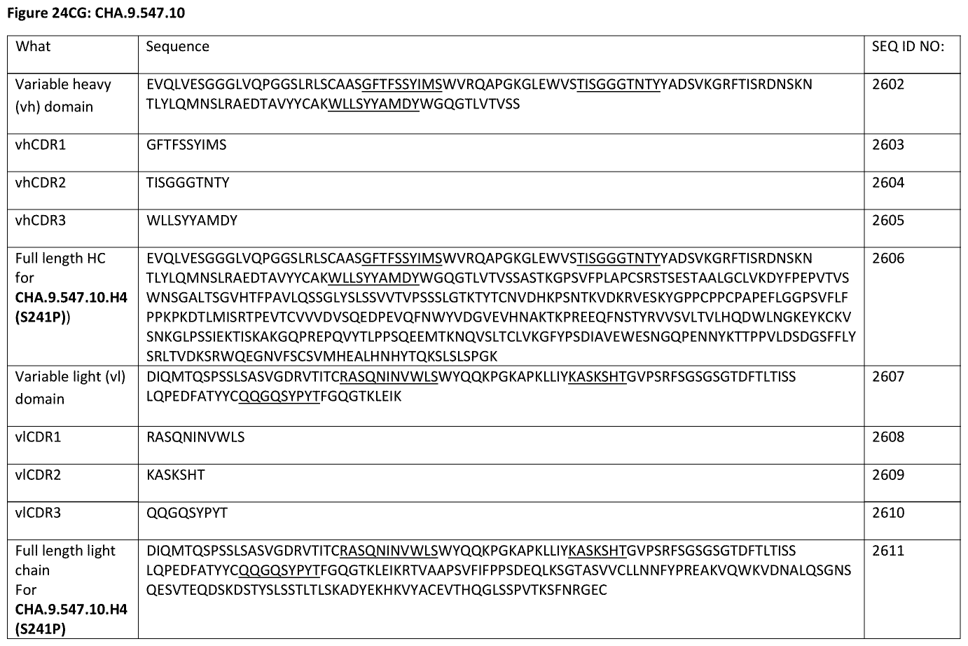

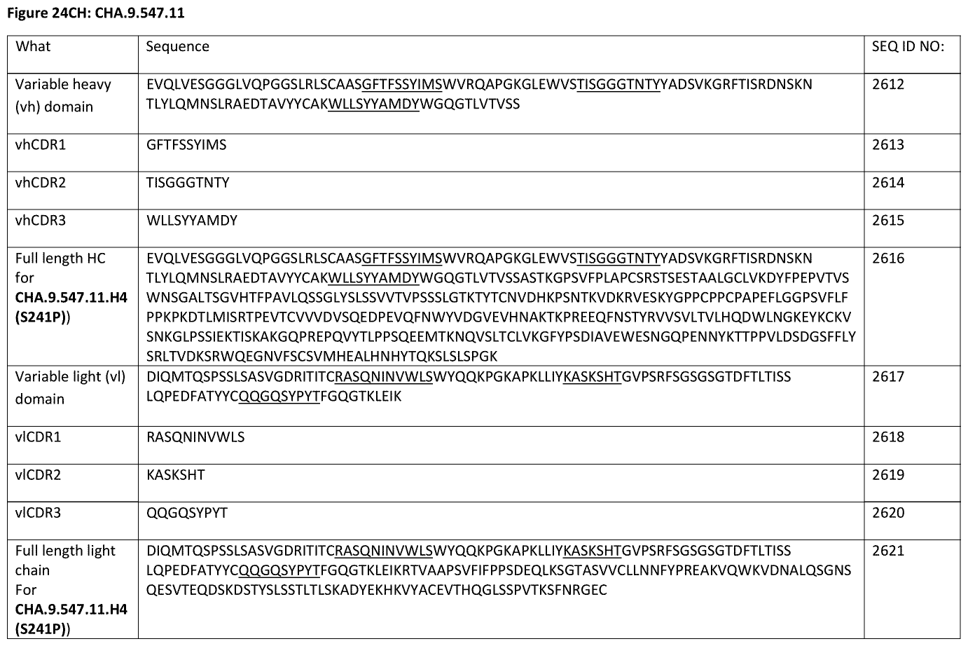

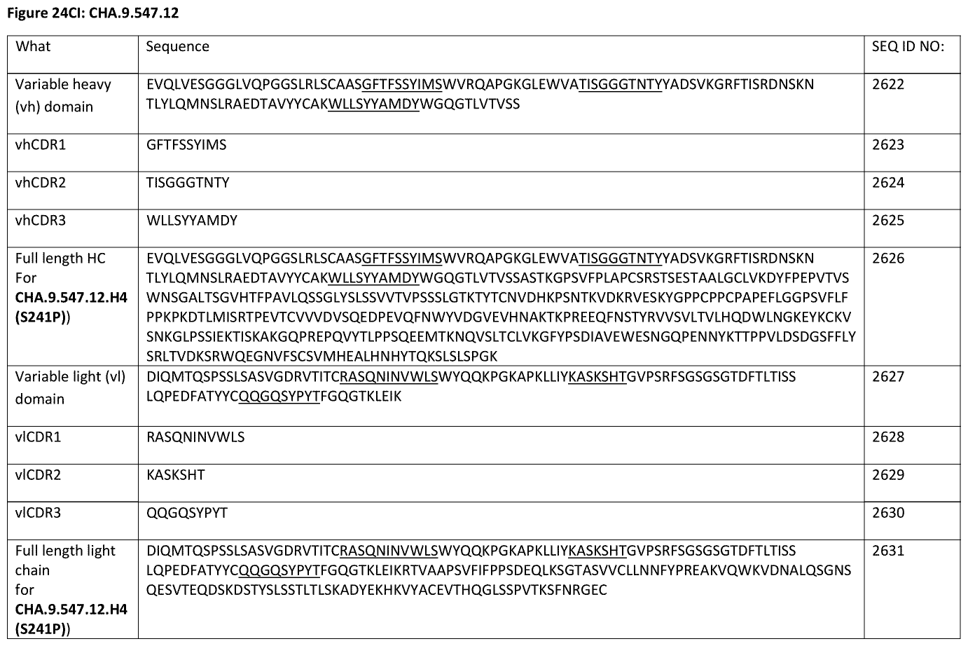

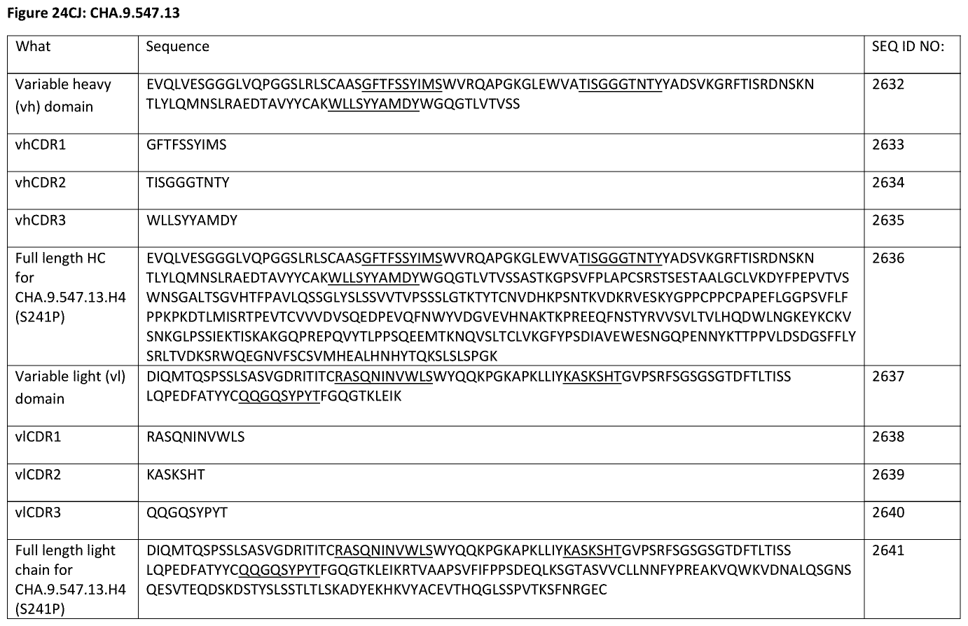

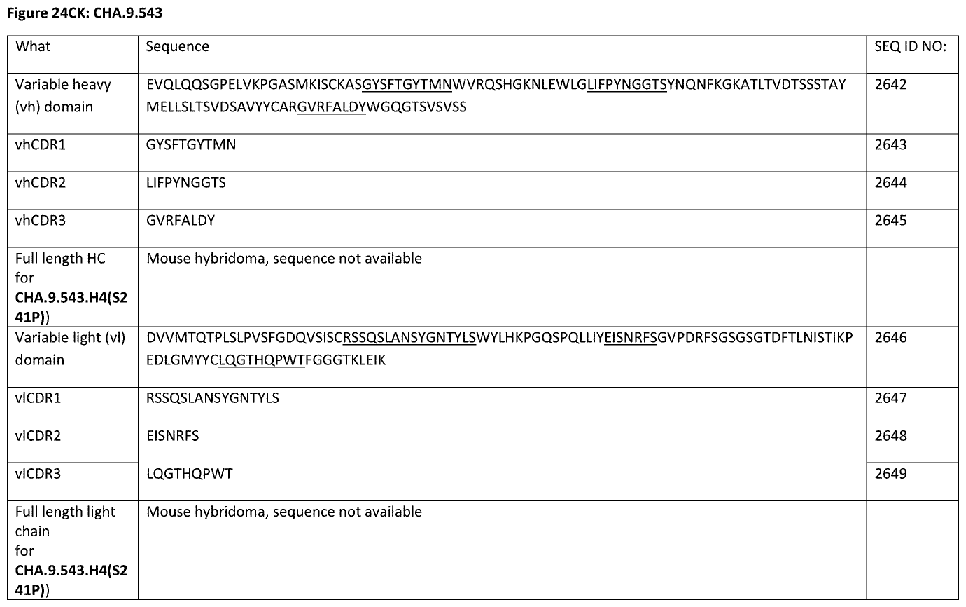

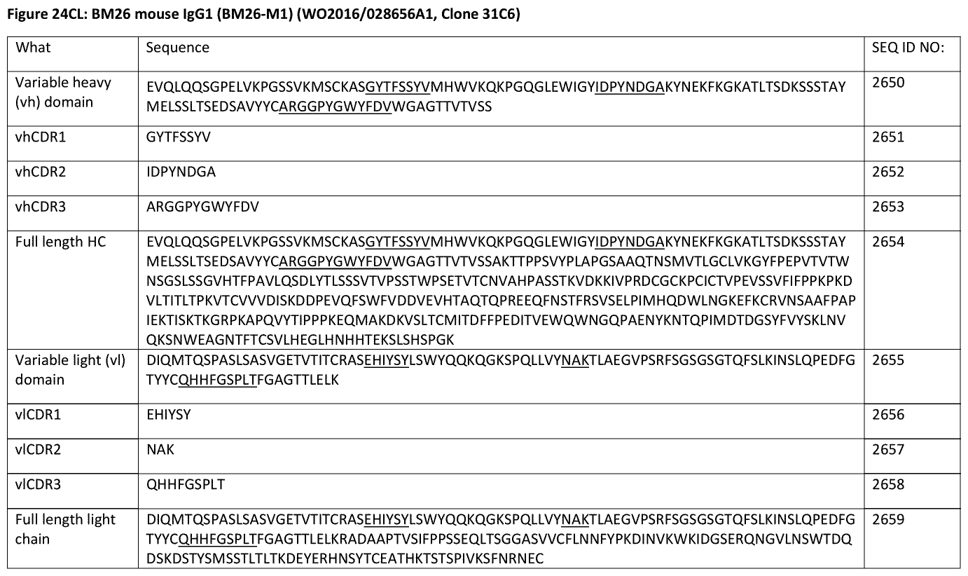

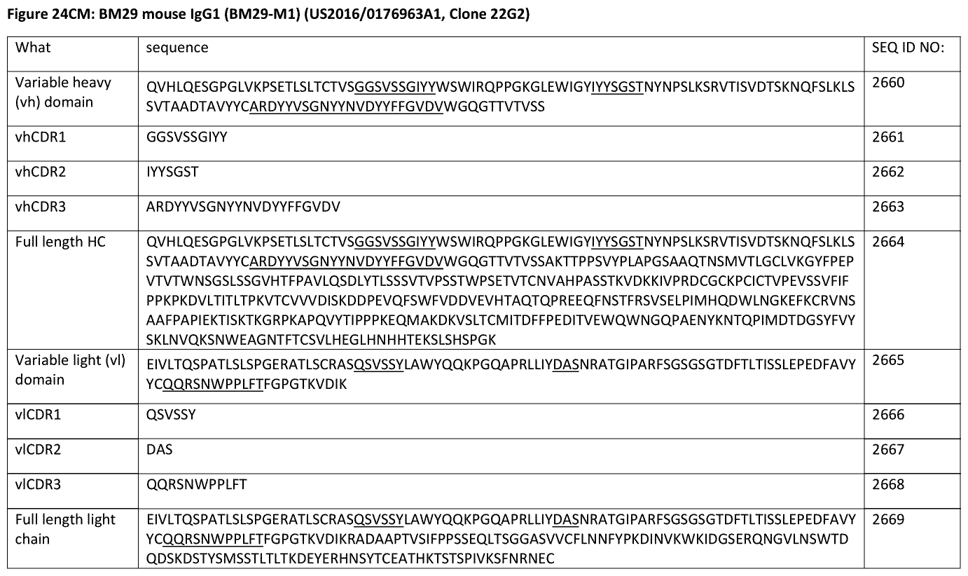

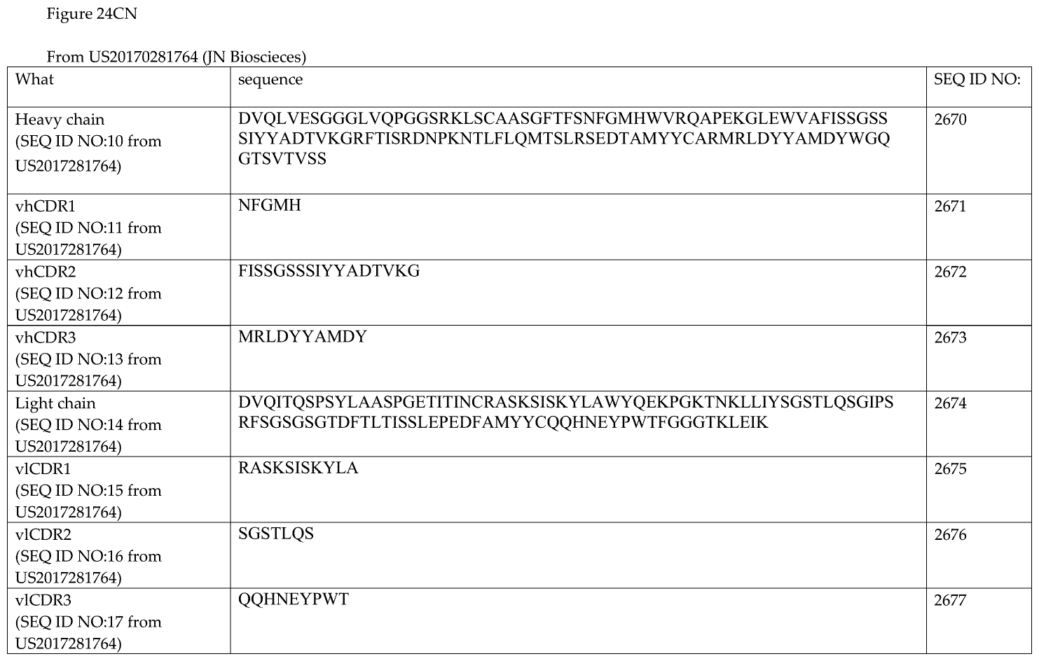

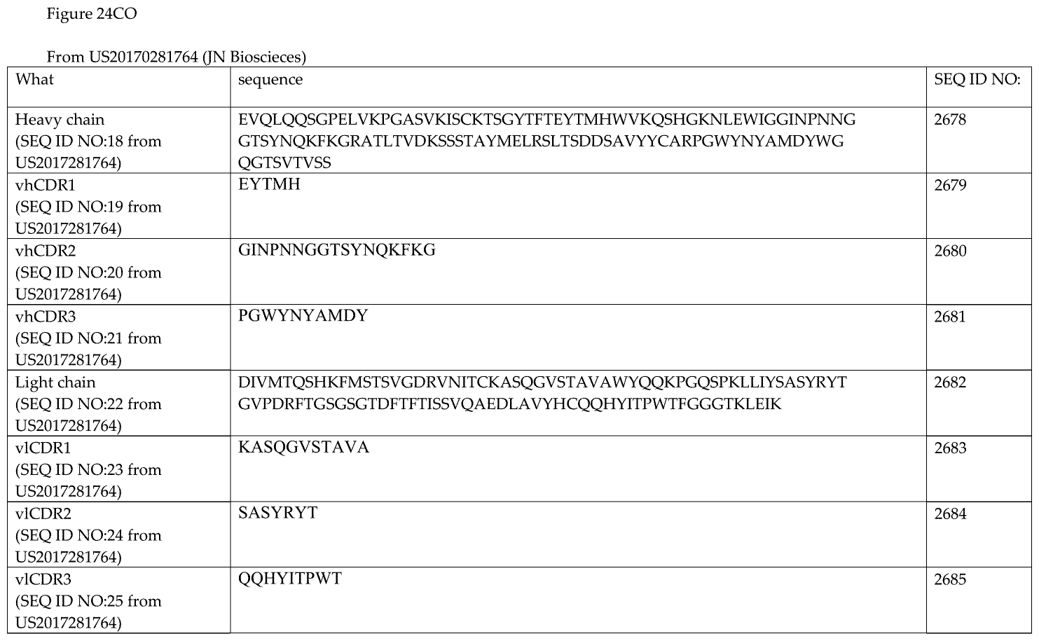

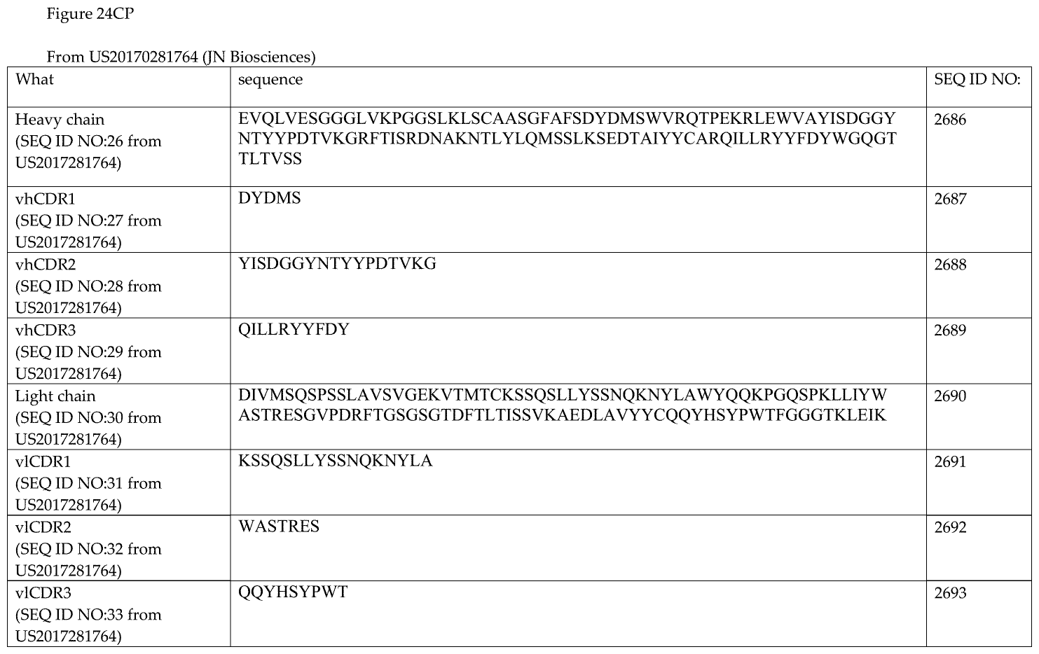

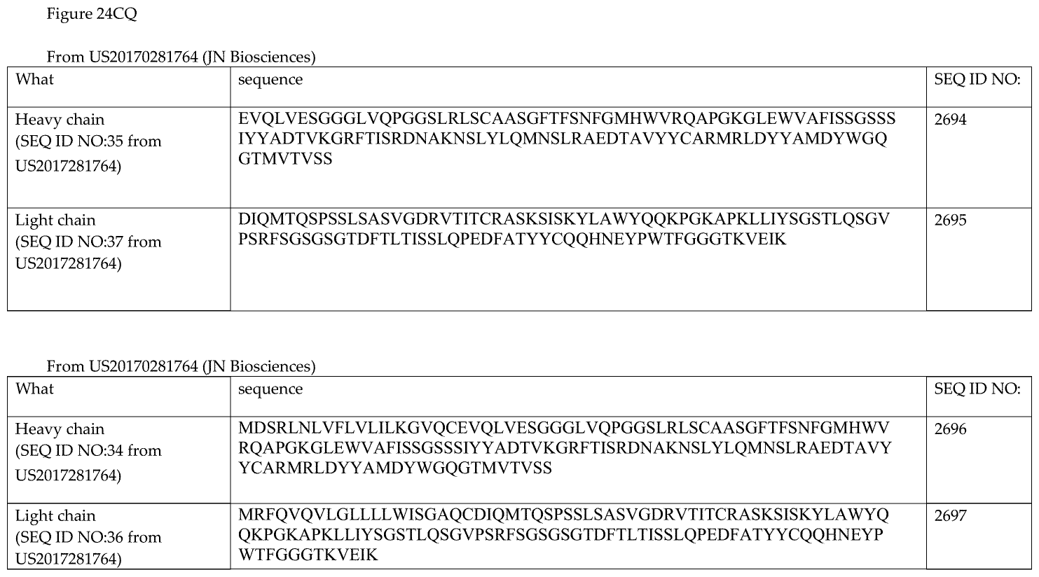

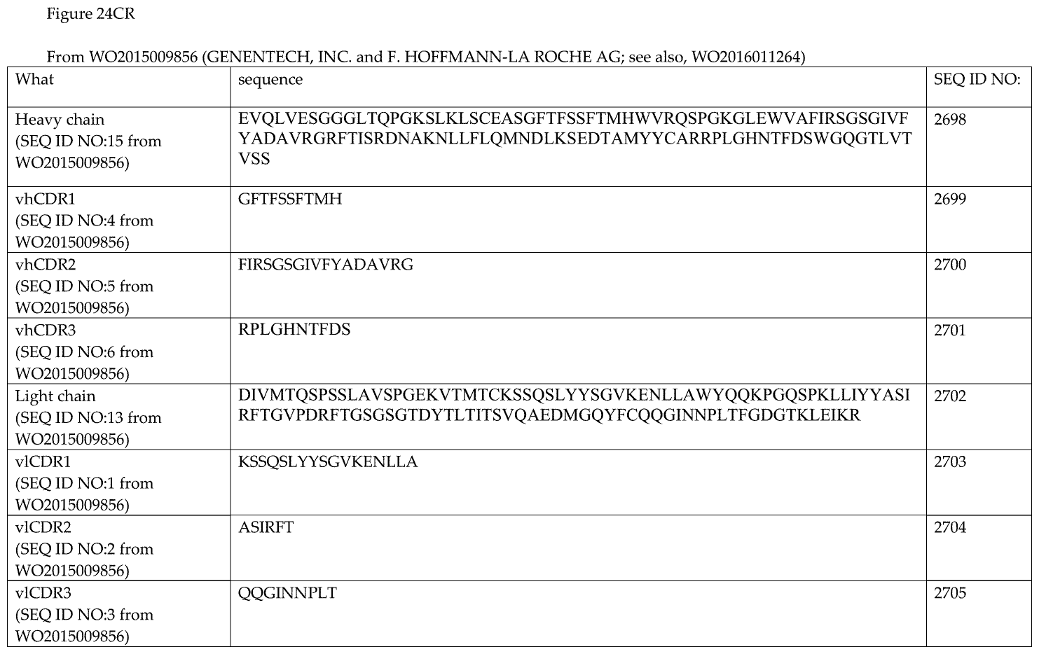

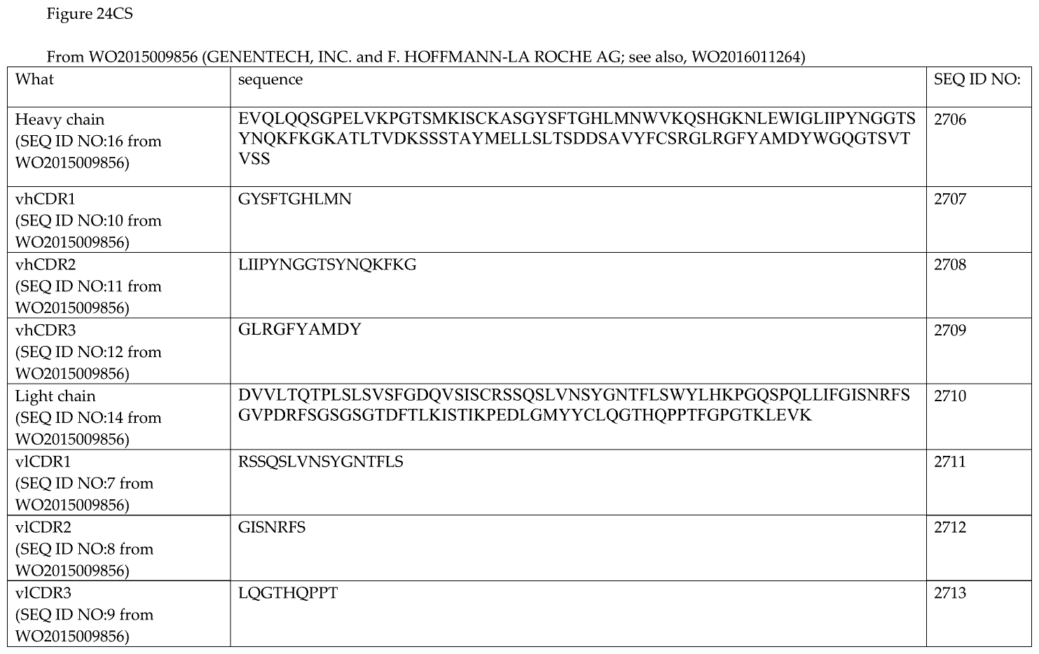

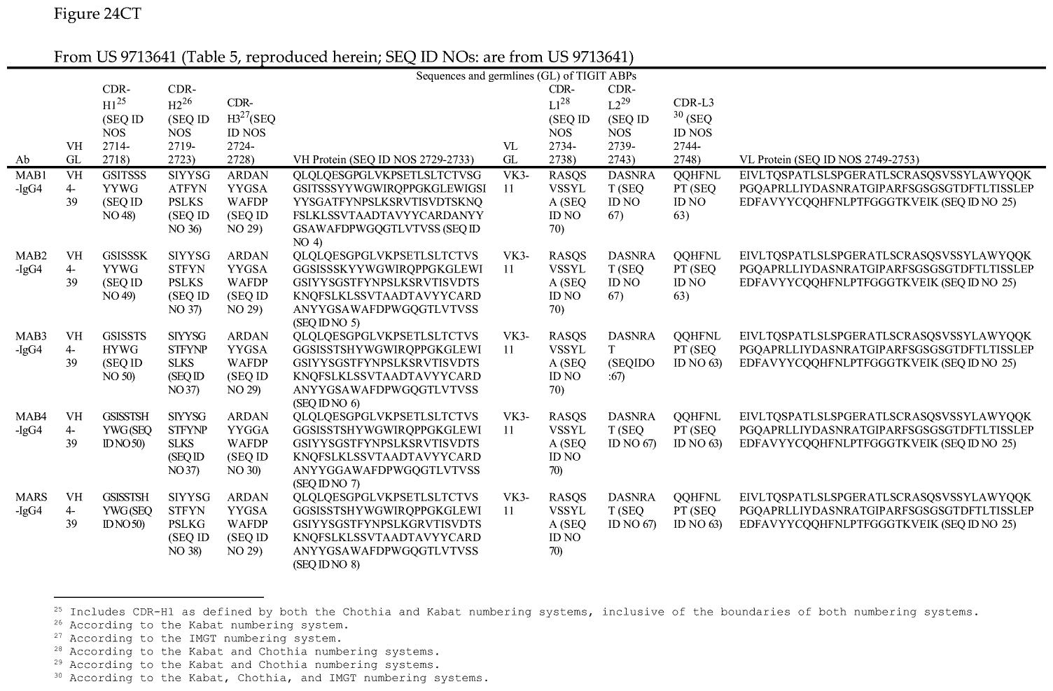

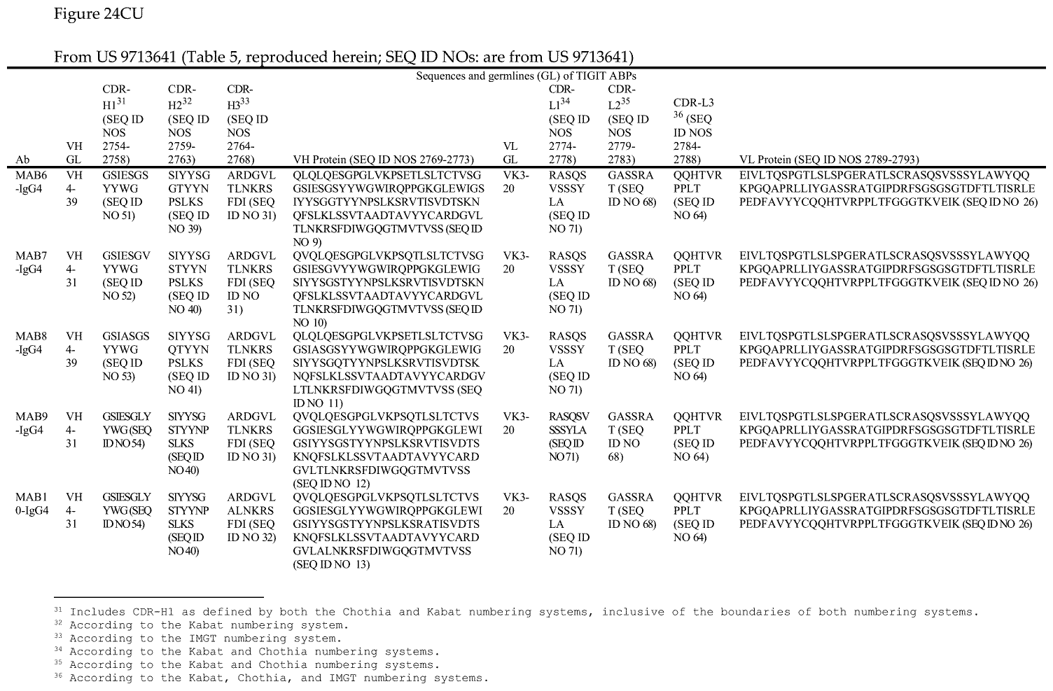

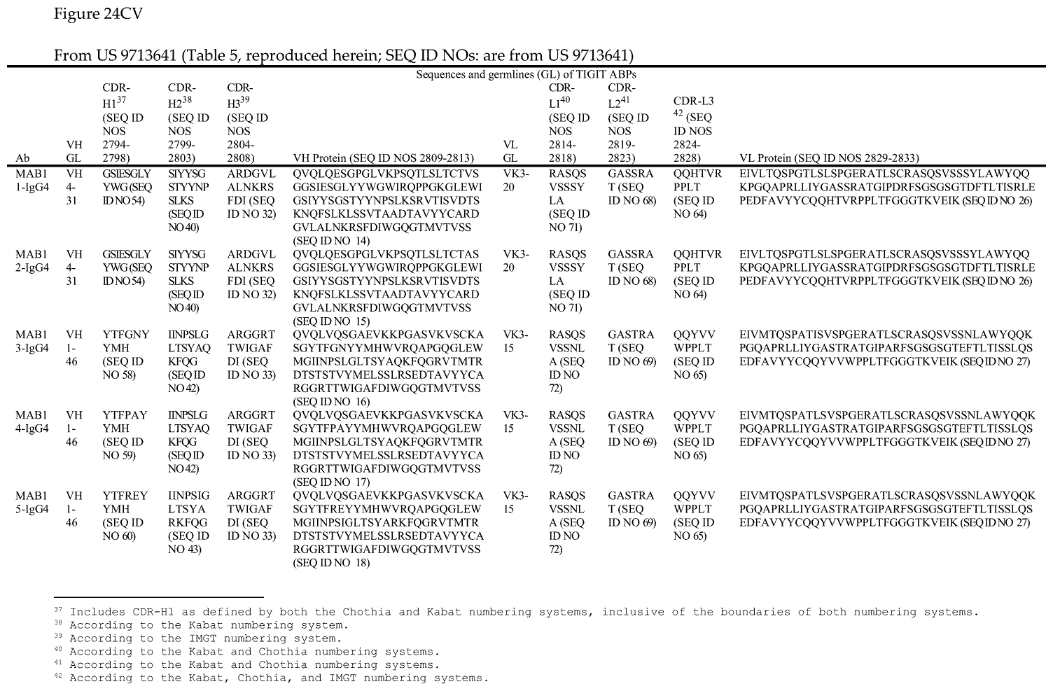

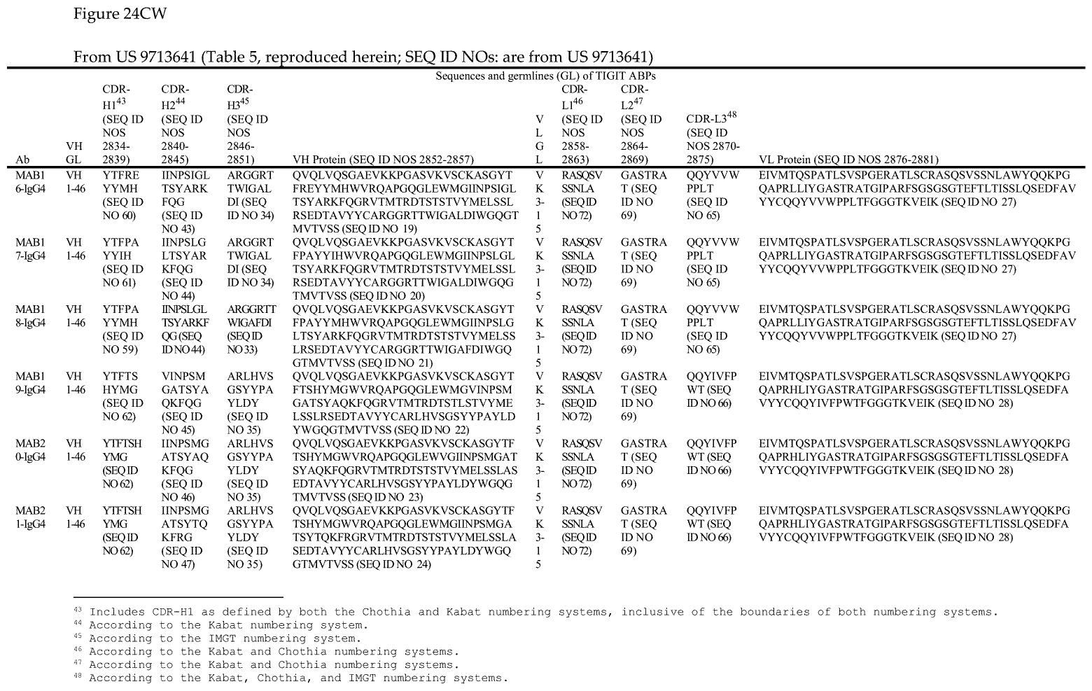

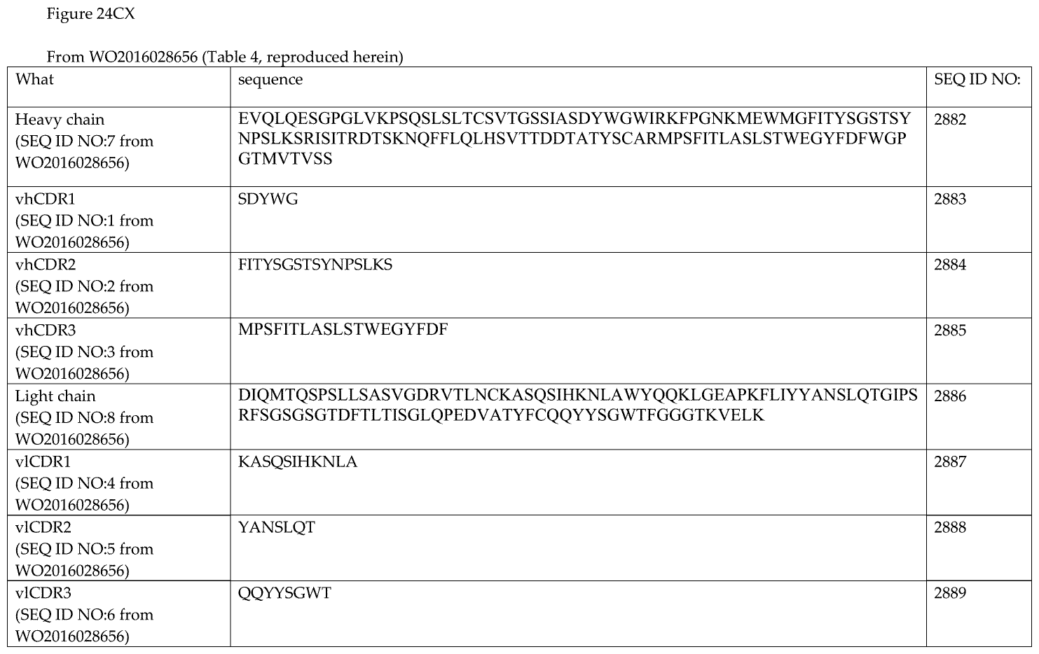

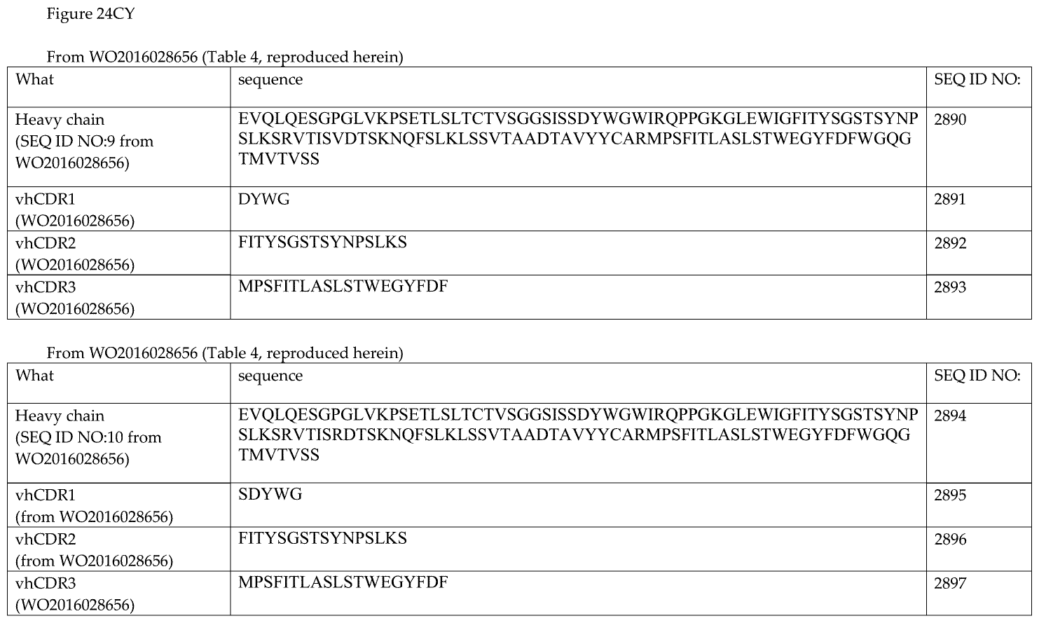

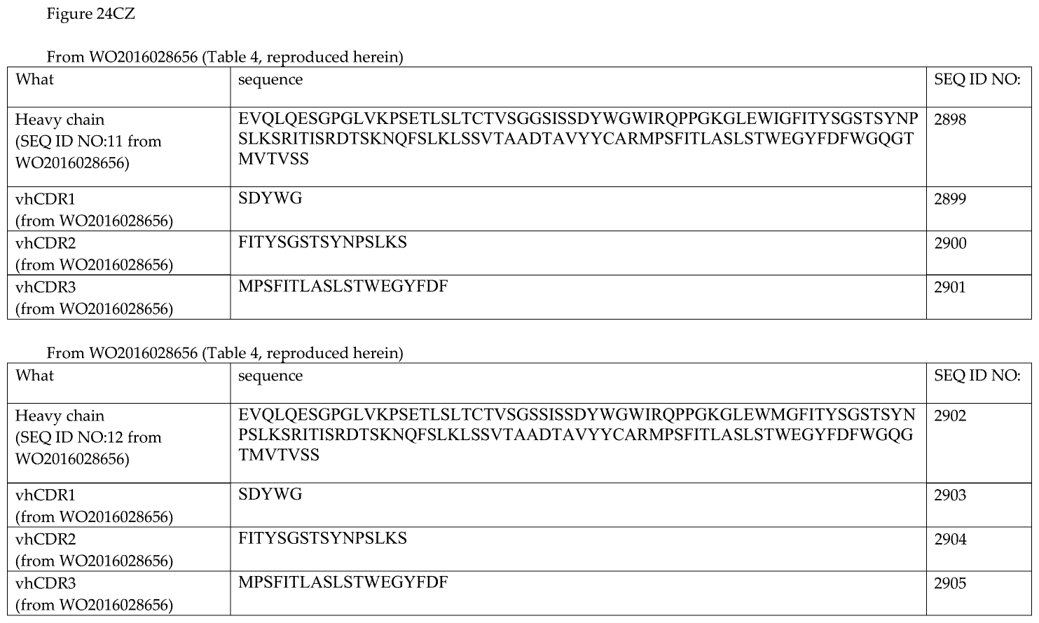

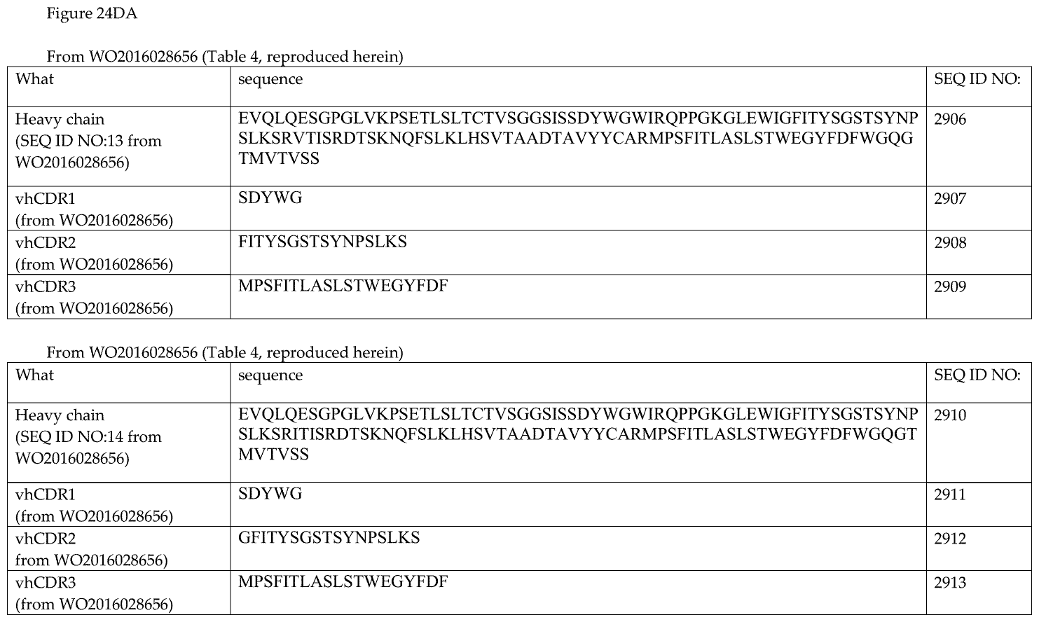

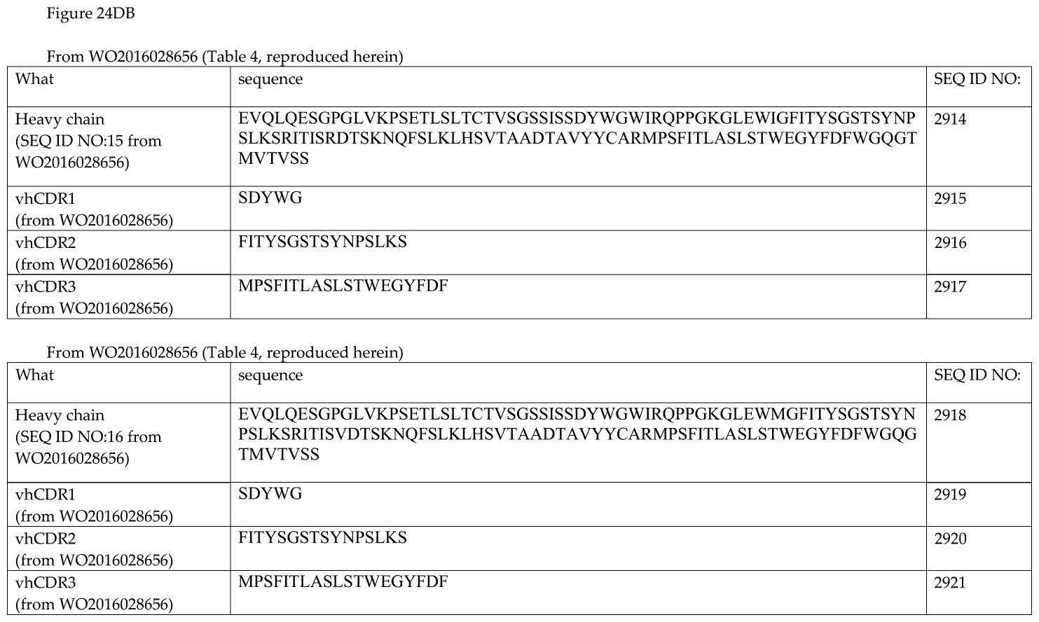

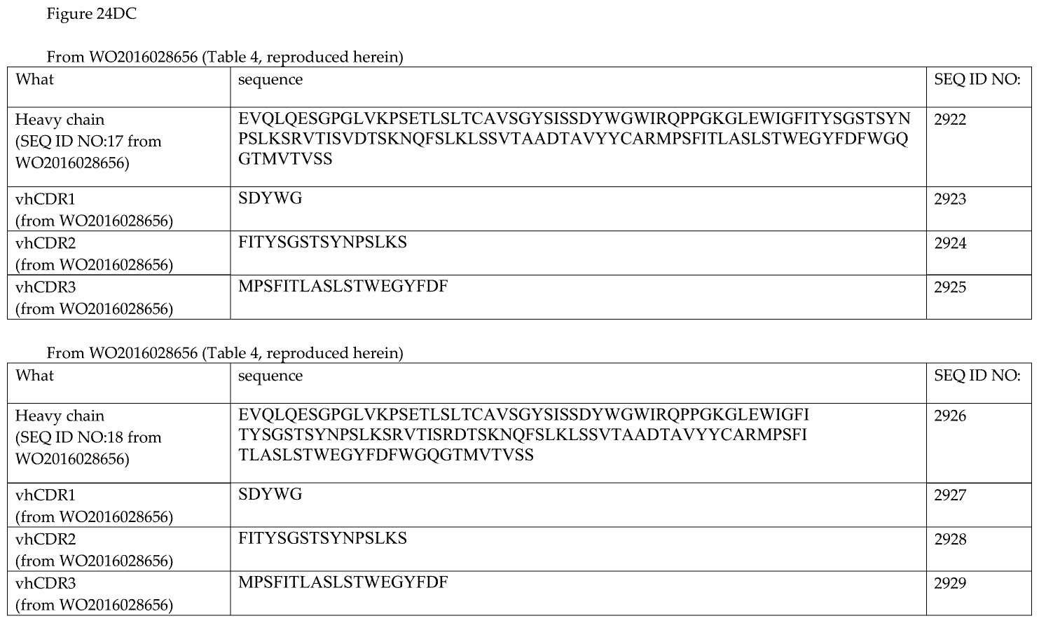

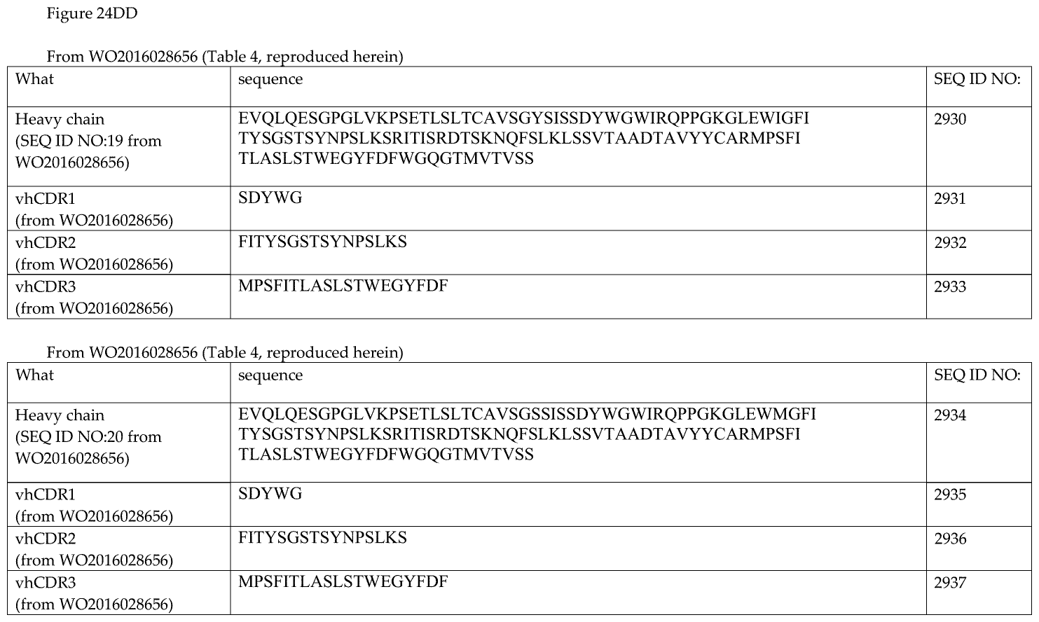

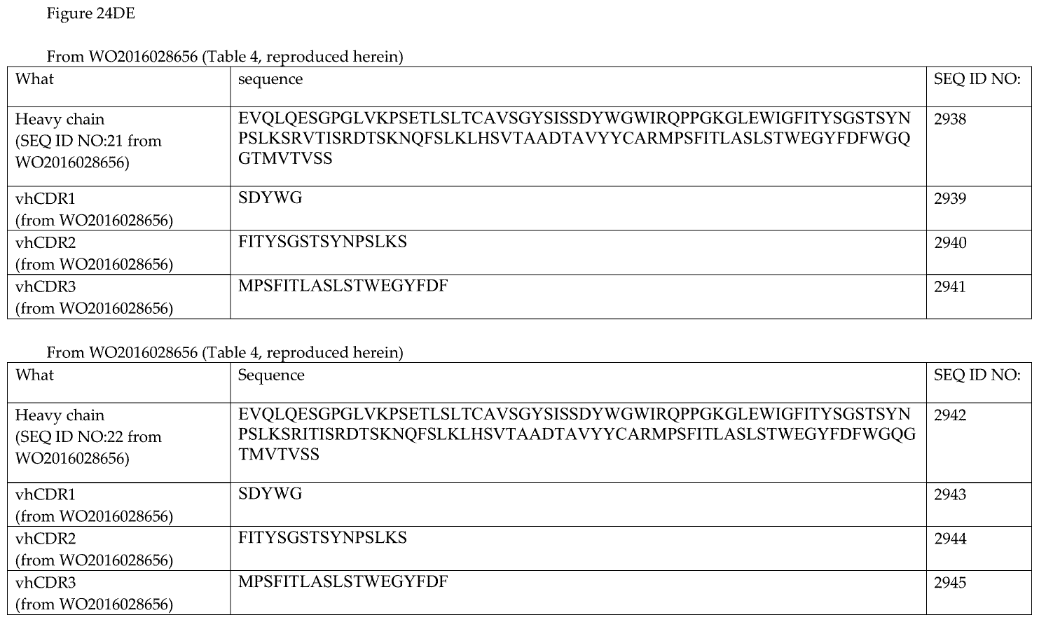

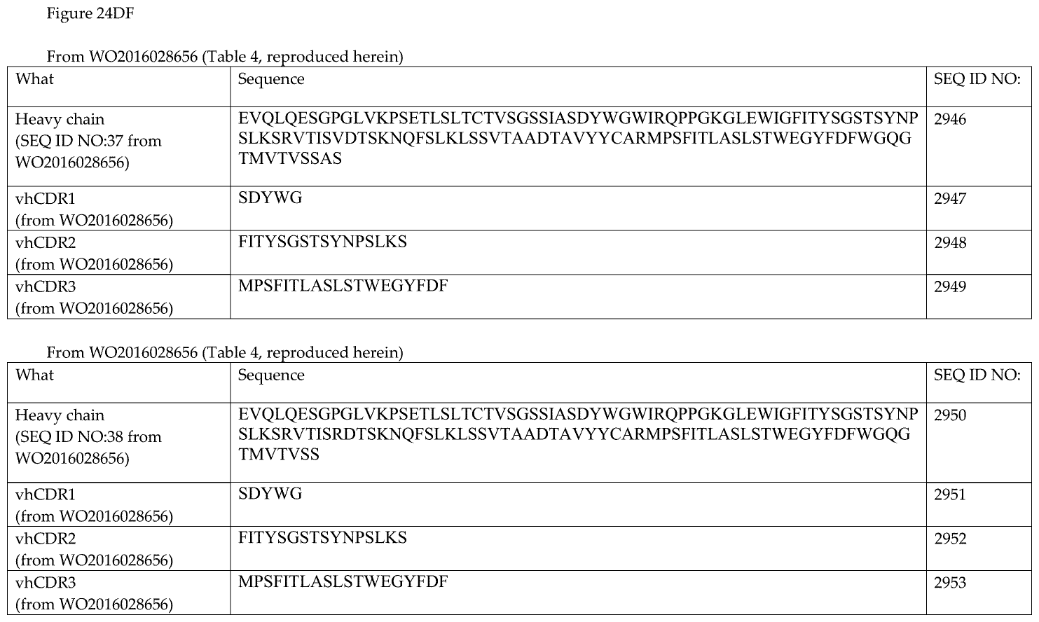

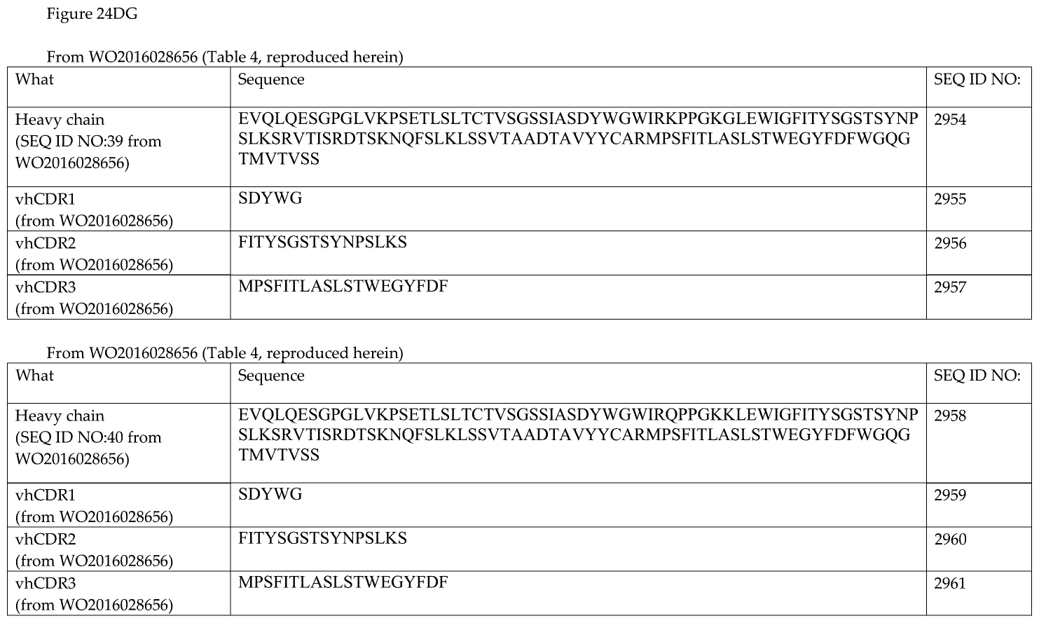

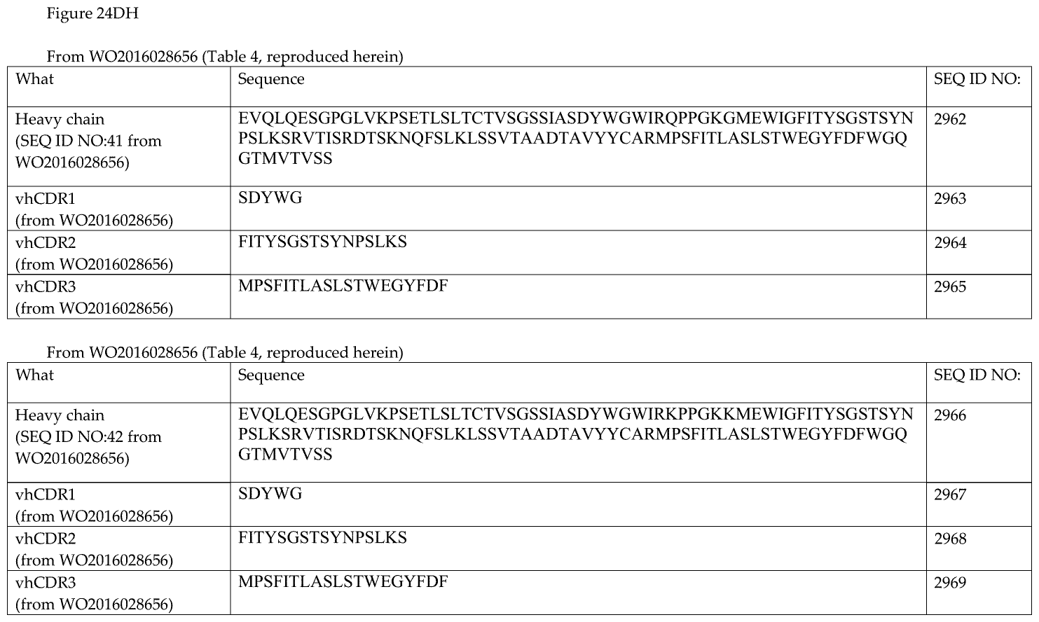

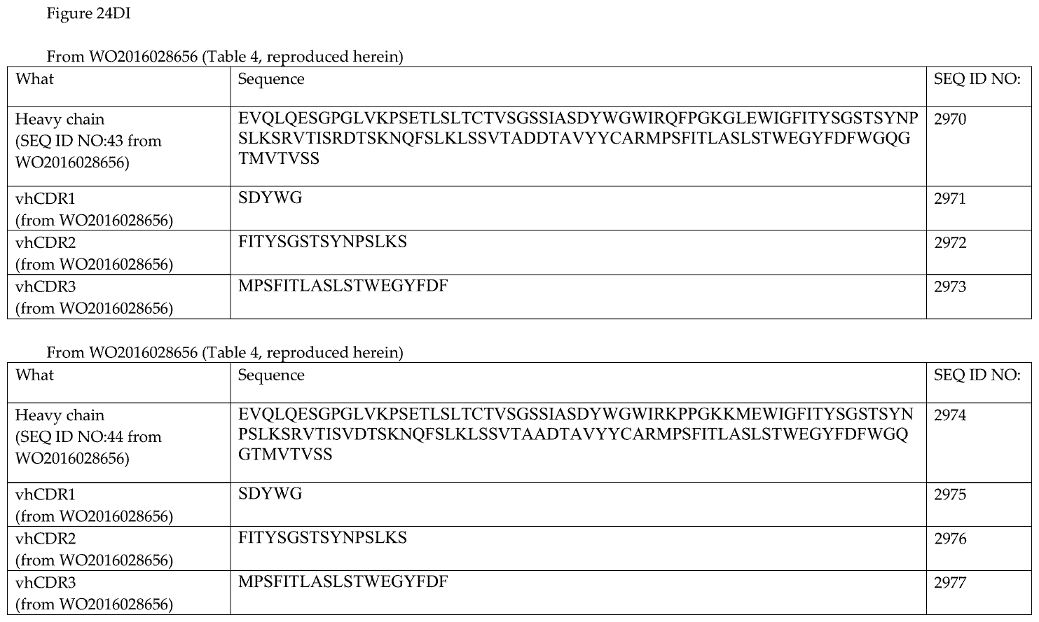

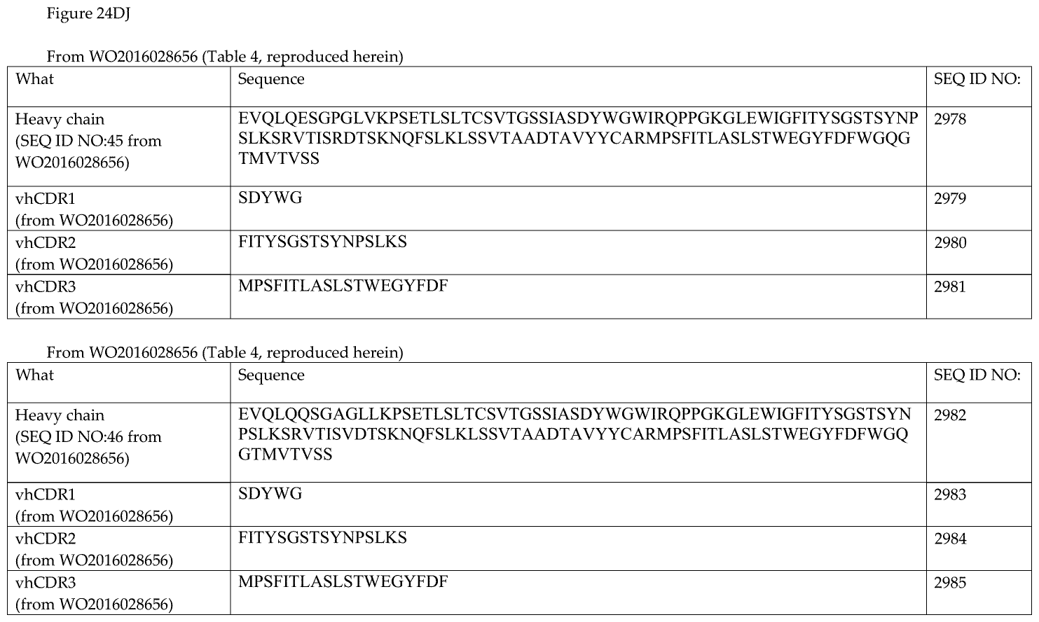

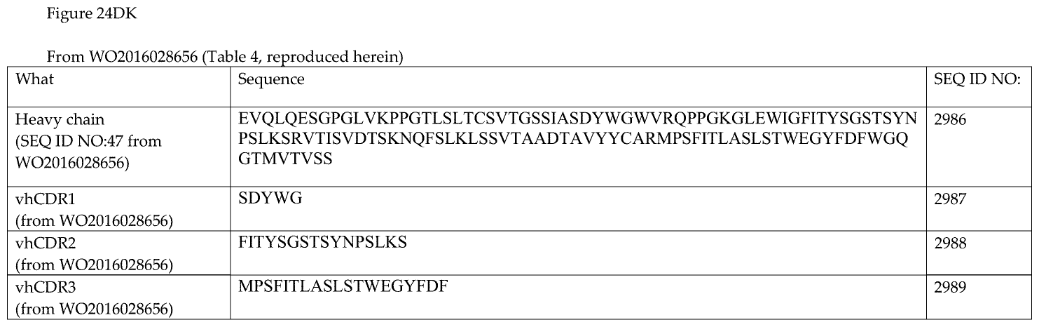

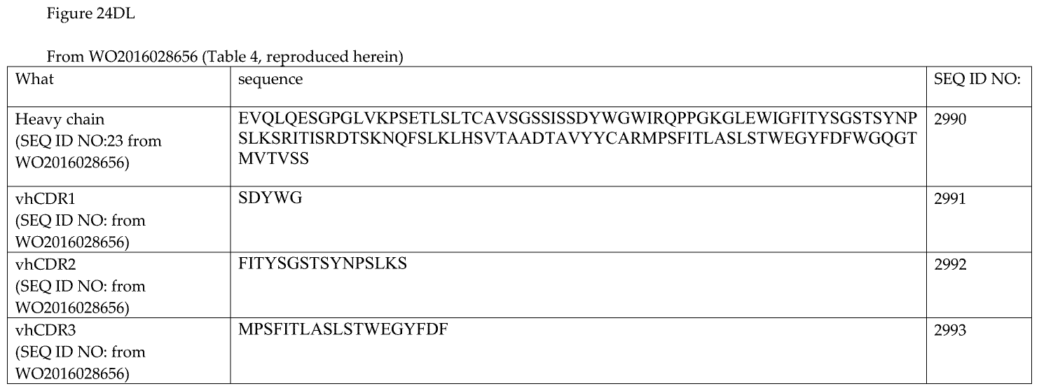

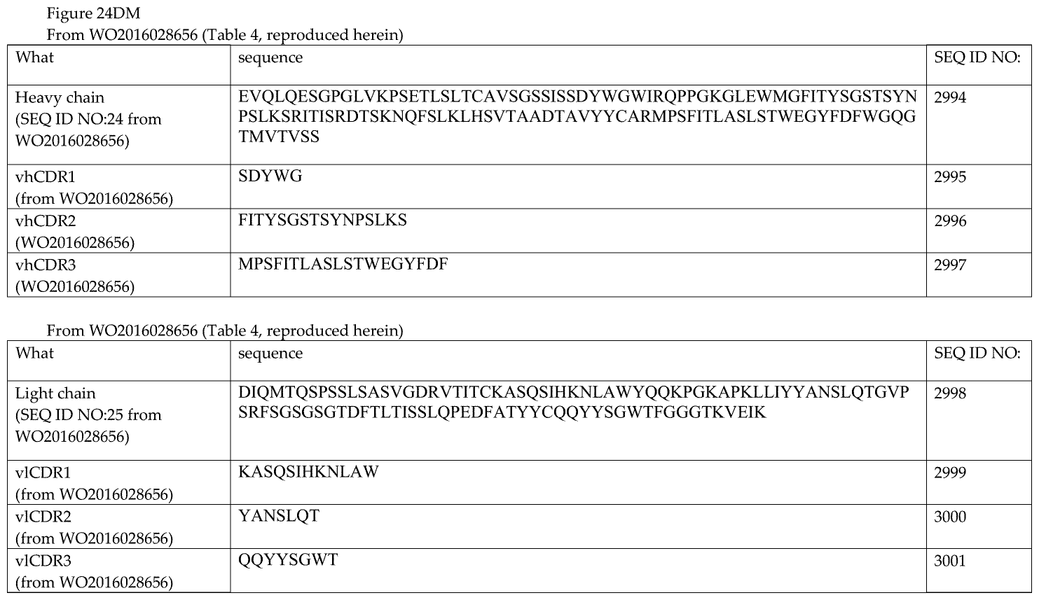

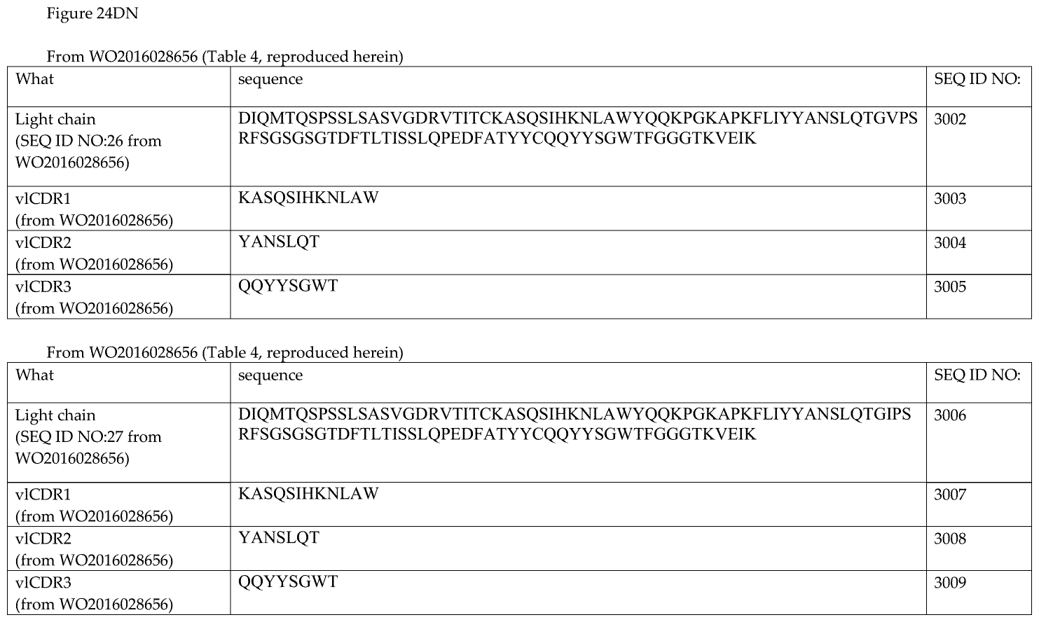

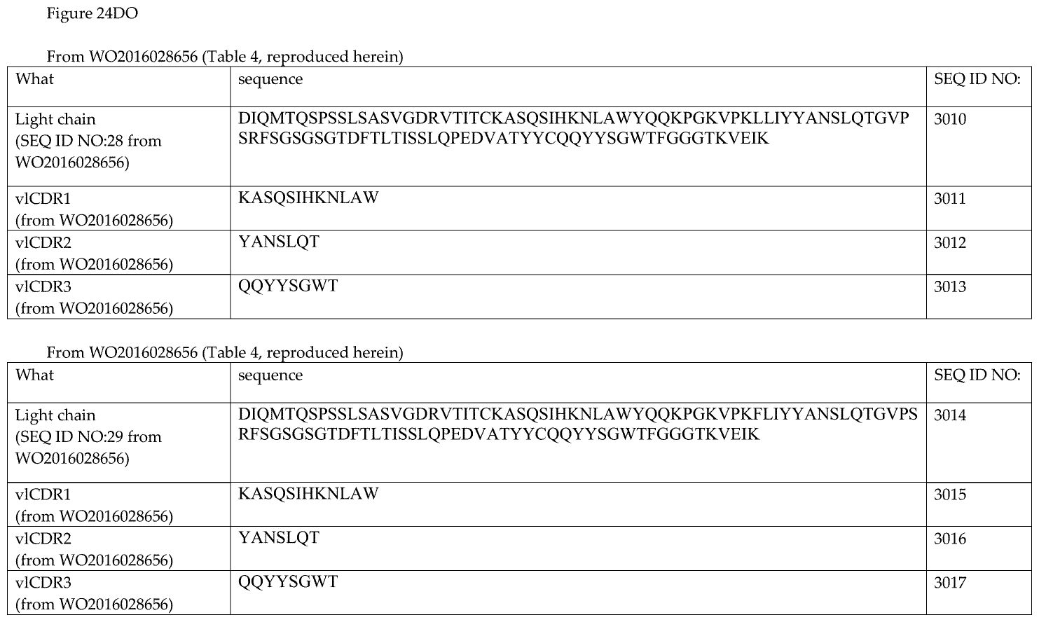

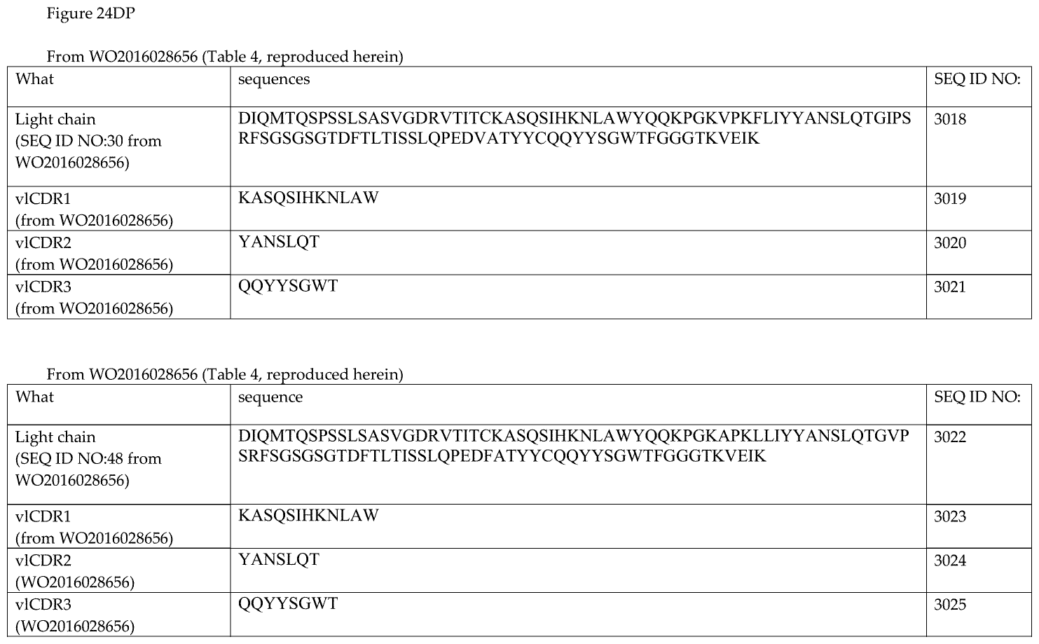

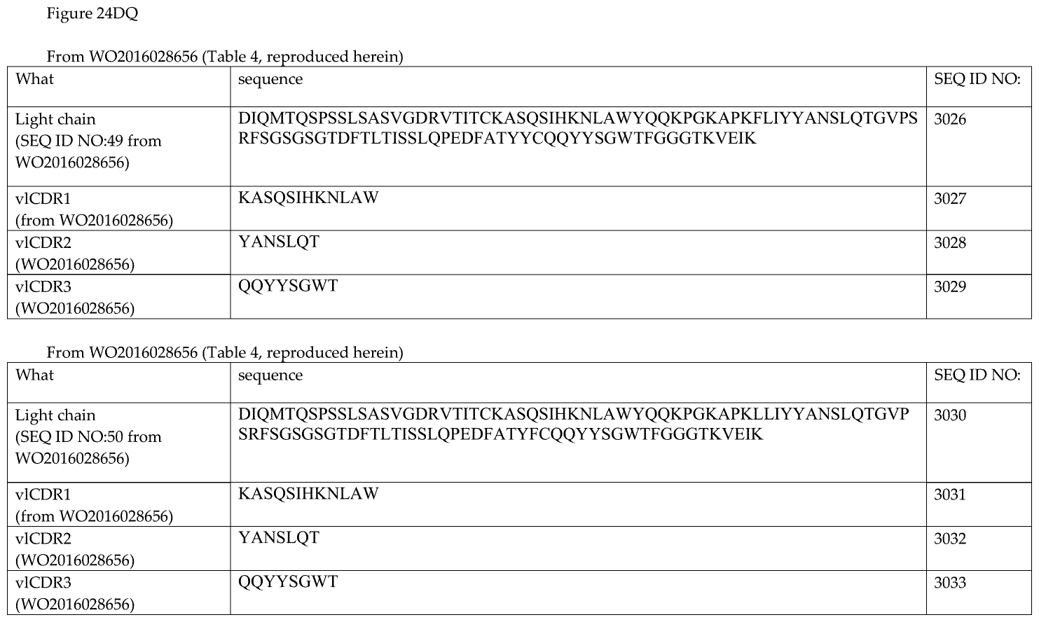

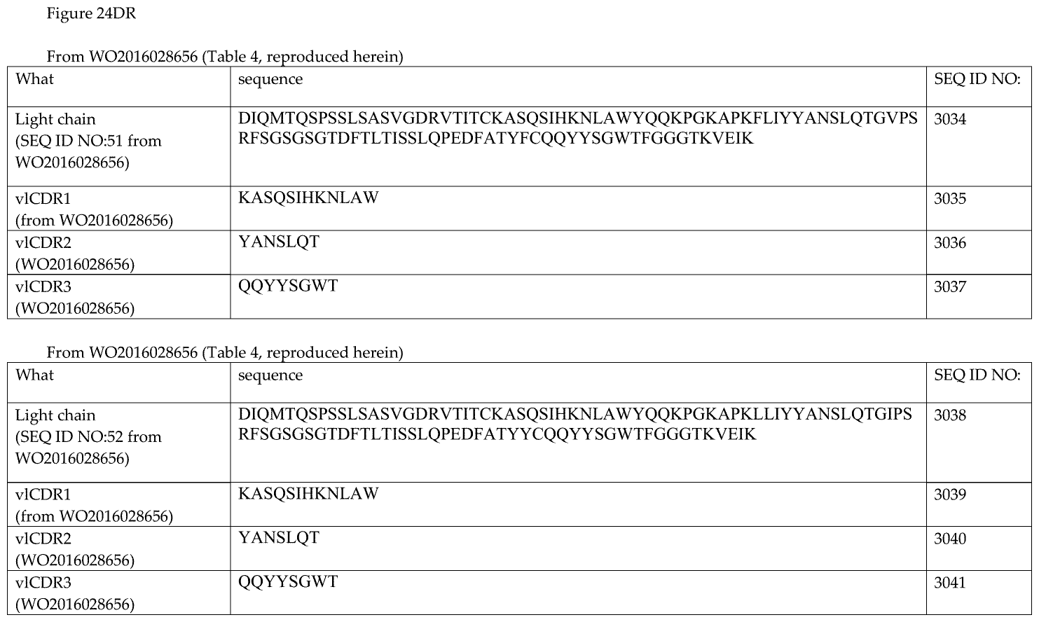

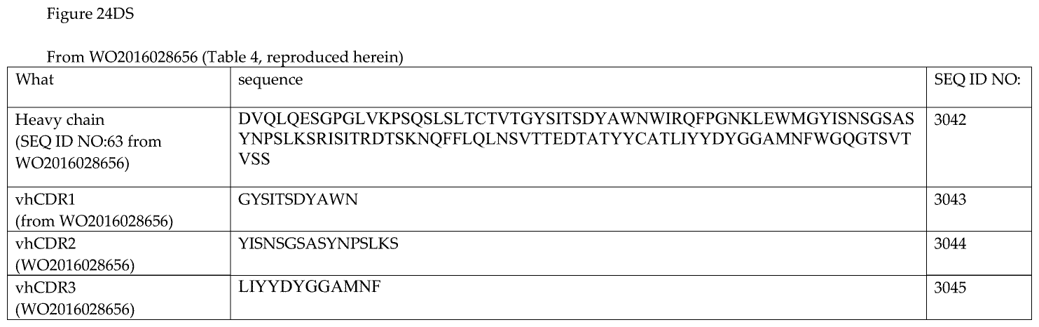

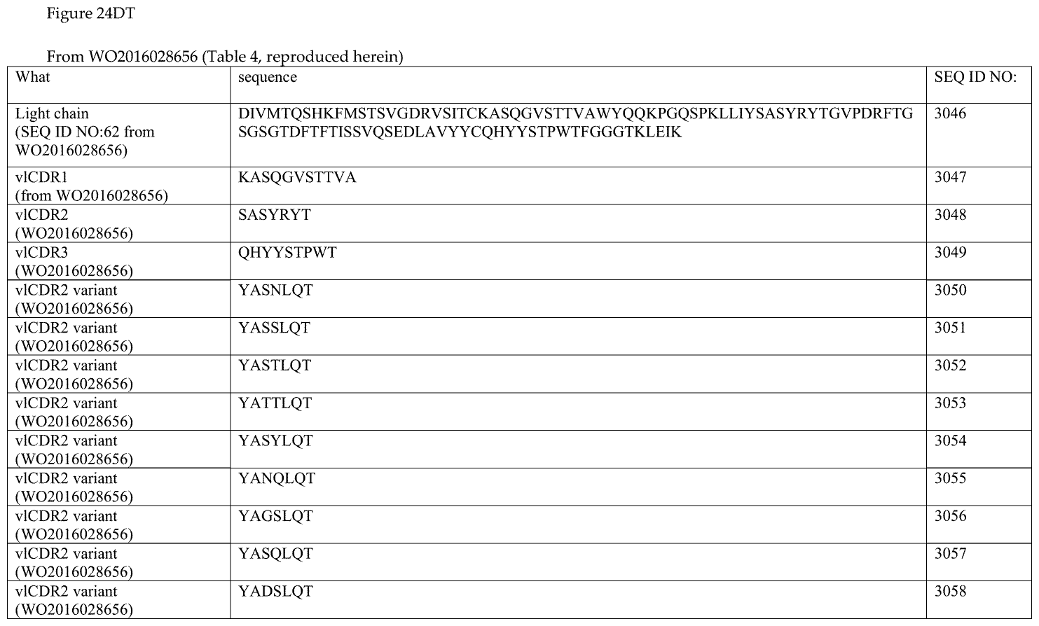

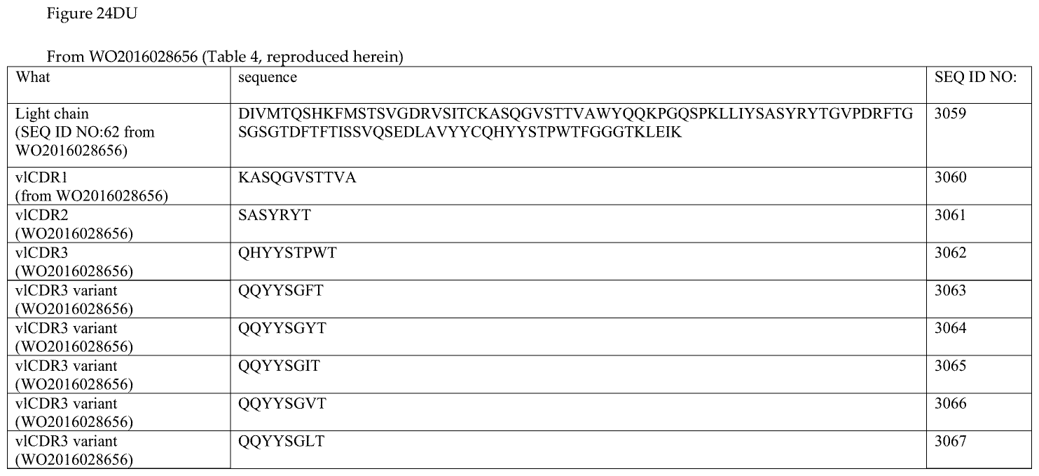

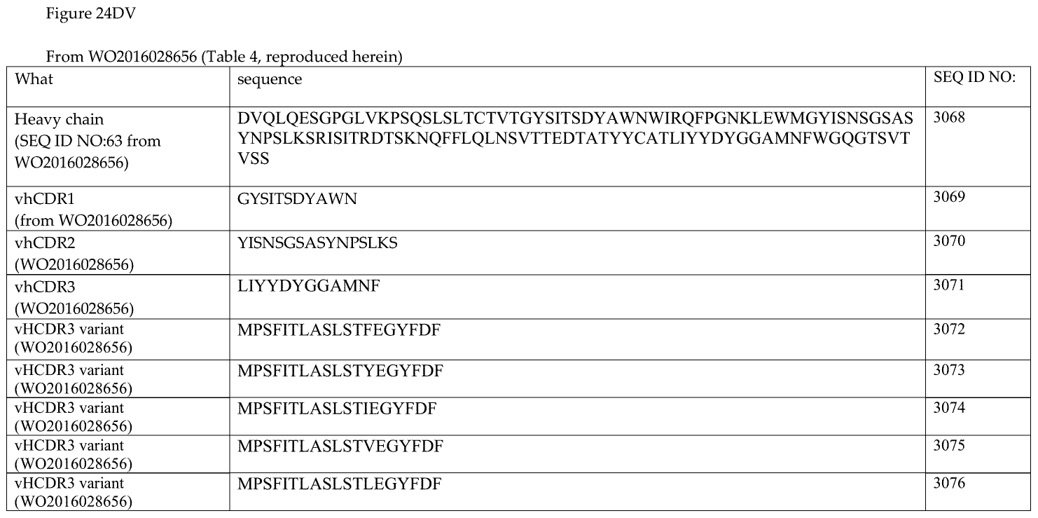

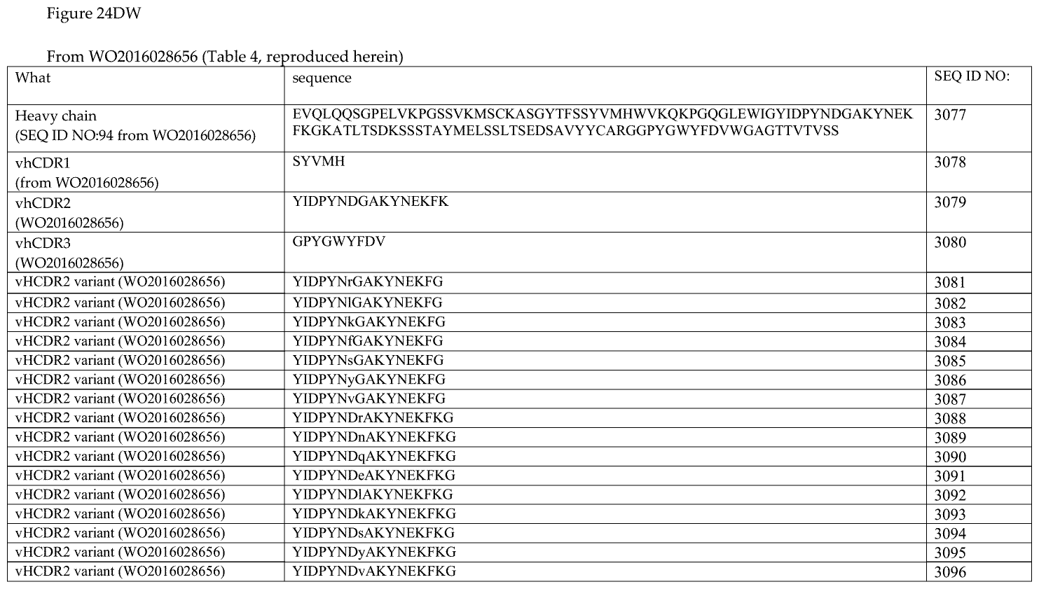

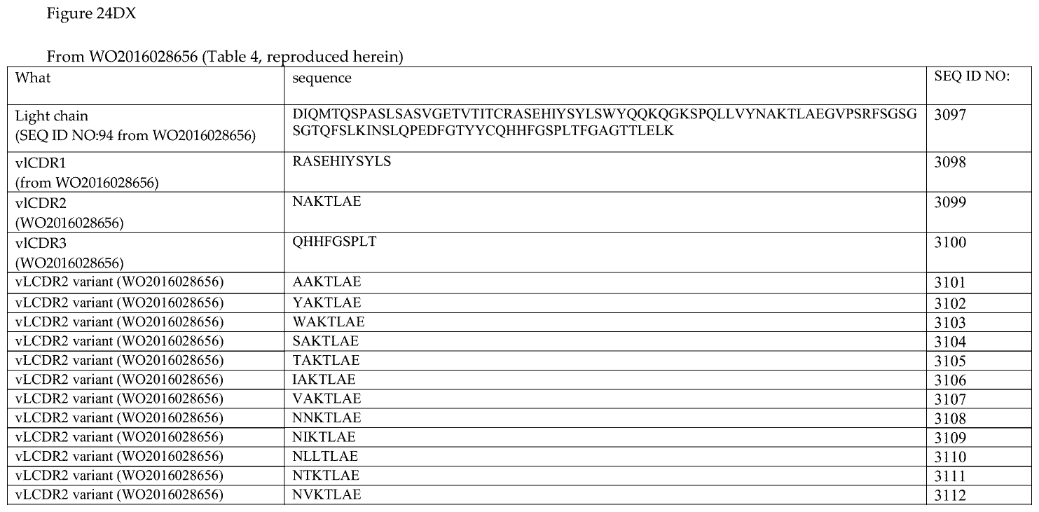

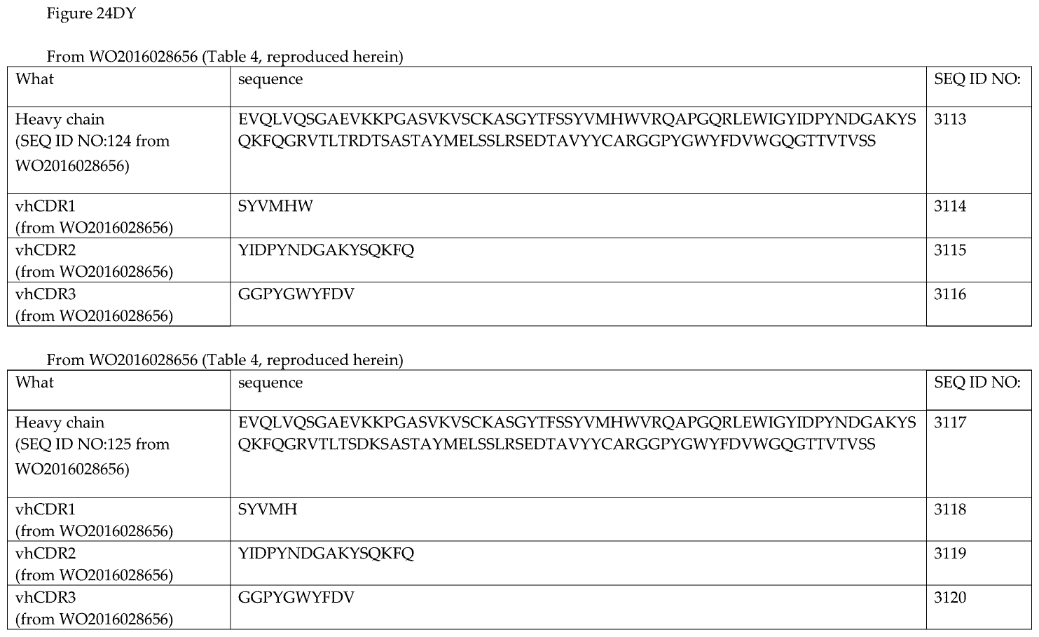

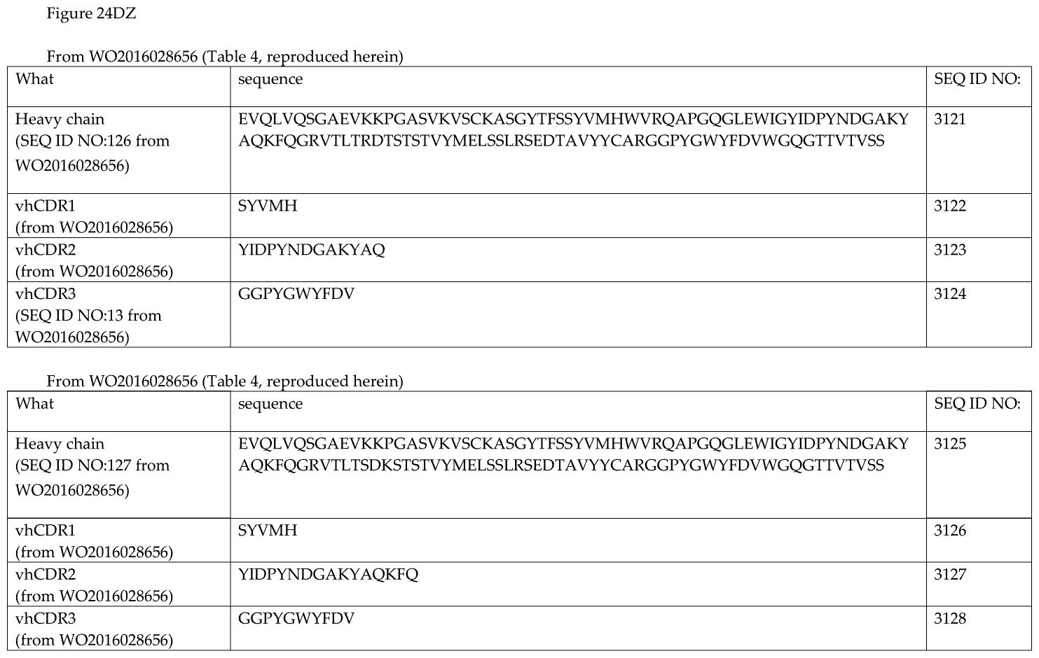

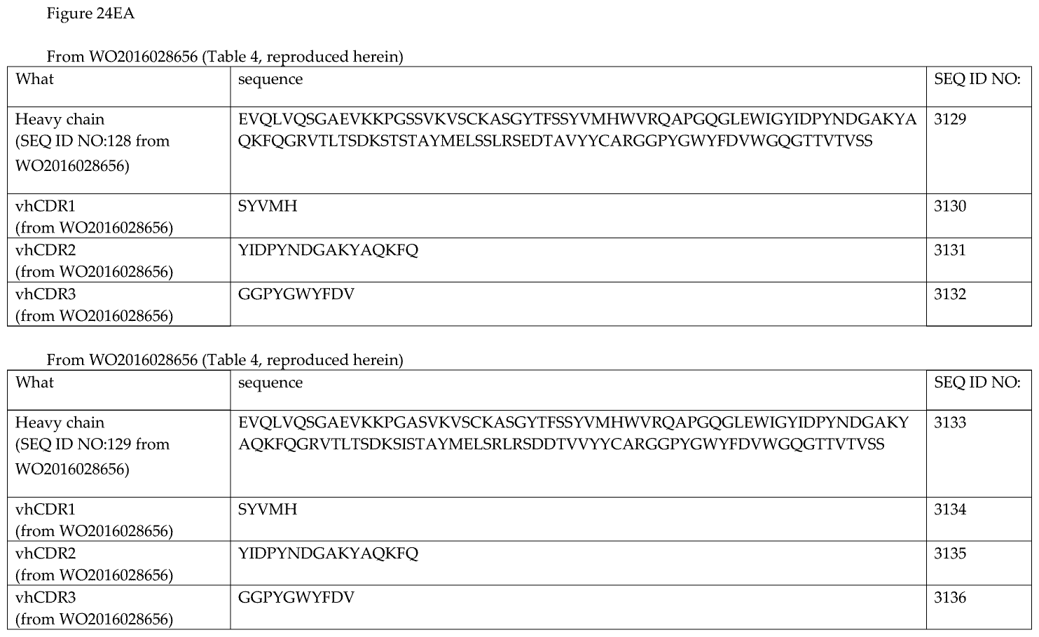

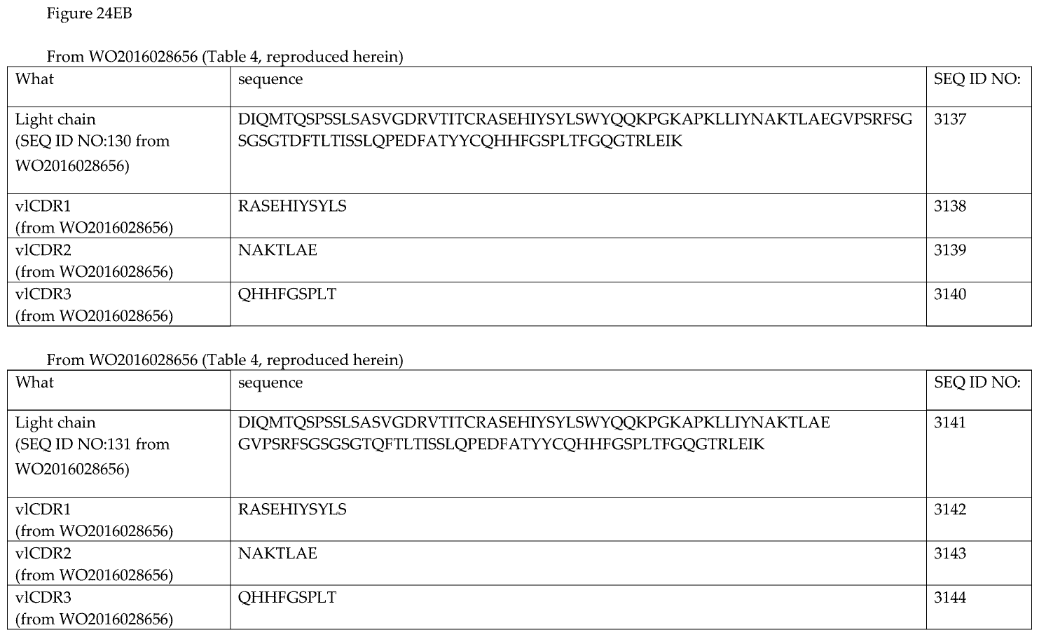

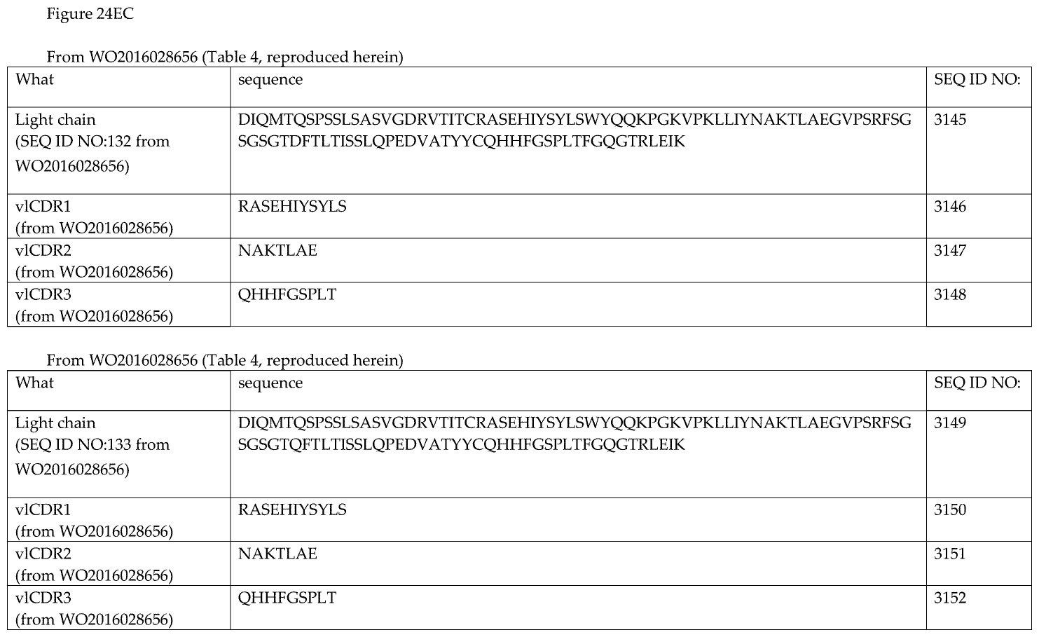

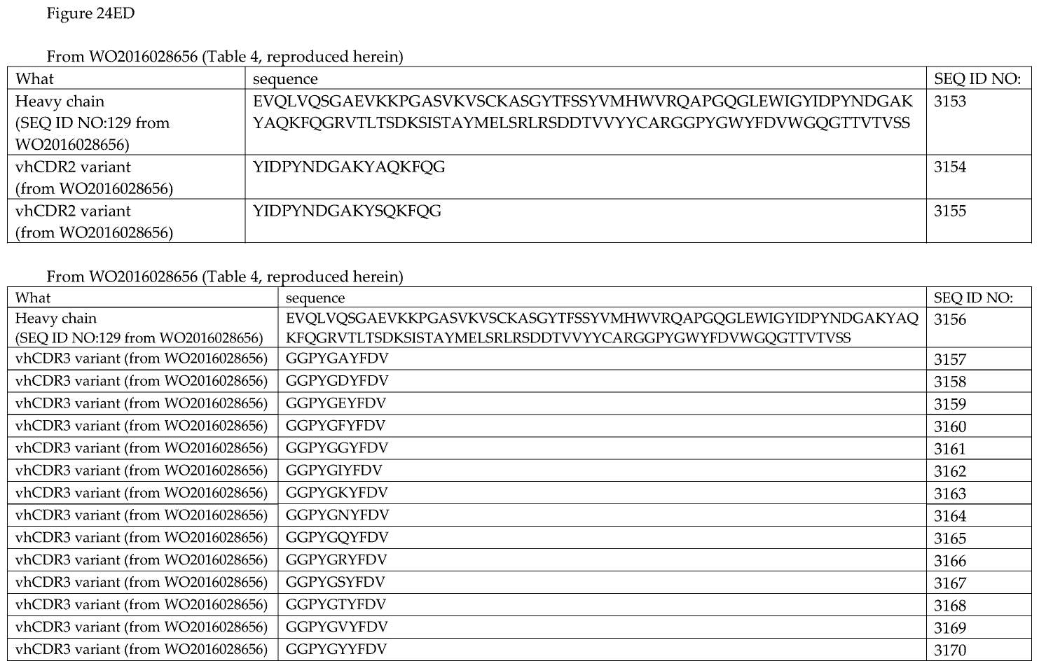

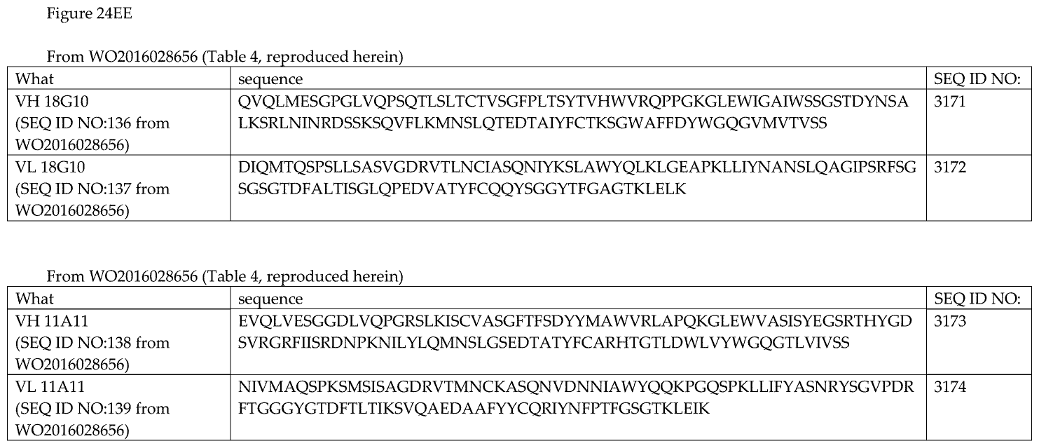

In some embodiments, the invention provides scFvs that bind to TIGIT comprising a variable heavy domain and a variable light domain linked by an scFv linker as outlined above. The VL and VH domains can be in either orientation, e.g. from N- to C-terminus “VH-linker-VL” or “VL-linker” VH″. These are named by their component parts; for example, “scFv-CPA. 9.086.VH-linker-VL” or “scFv-CPA.9.086.VL-linker-VH.” Thus, “scFv-CPA.9.086” can be in either orientation. The anti-PVRIG/anti-TIGIT bispecific antibodies of the invention can comprise any scFvs that bind to TIGIT as part of the TIGIT binding portion of the anti-PVRIG/anti-TIGIT bispecific antibodies. The anti-PVRIG/anti-TIGIT bispecific antibodies of the invention can comprise any scFvs that bind to TIGIT as part of the TIGIT antigen binding domain of the anti-PVRIG/anti-TIGIT bispecific antibodies. In some embodiments, anti-PVRIG/anti-TIGIT bispecific antibody comprises the PVRIG sequences provided in FIG. 26A-26B as the PVRIG binding portion. In many embodiments, the antibodies of the invention are human (derived from phage) and block binding of TIGIT and PVR. As shown in FIGS. 3 and 4A-4AA, the CPA antibodies that both bind and block the receptor-ligand interaction are as below, with their components outlined as well (as discussed in the “Sequence” section, the sequences of all but the scFv constructs are in the sequence listing as well as provided in FIG. 24A-24EE):

CPA.9.018, CPA.9.018.VH, CPA.9.018.VL, CPA.9.018.HC, CPA.9.018.LC, CPA.9.018.H1, CPA.9.018.H2, CPA.9.018.H3, CPA.9.018.H4; CPA.9.018.H4(S241P); CPA.9.018.vhCDR1, CPA.9.018.vhCDR2, CPA.9.018.vhCDR3, CPA.9.018.vlCDR1, CPA.9.018.vlCDR2, CPA.9.018.vlCDR3 and scFv-CPA.9.018;

CPA.9.027, CPA.9.027.VH, CPA.9.027.VL, CPA.9.027.HC, CPA.9.027.LC, CPA.9.027.H1, CPA.9.027.H2, CPA.9.027.H3, CPA.9.027.H4; CPA.9.018.H4(S241P); CPA.9.027.vhCDR1, CPA.9.027.vhCDR2, CPA.9.027.vhCDR3, CPA.9.027.vlCDR1, CPA.9.027.vlCDR2, CPA.9.027.vlCDR3 and scFv-CPA.9.027;

CPA.9.049, CPA.9.049.VH, CPA.9.049.VL, CPA.9.049.HC, CPA.9.049.LC, CPA.9.049.H1, CPA.9.049.H2, CPA.9.049.H3; CPA.9.049.H4; CPA.9.049.H4(S241P); CPA.9.049.vhCDR1, CPA.9.049.vhCDR2, CPA.9.049.vhCDR3, CPA.9.049.vlCDR1, CPA.9.049.vlCDR2, CPA.9.049.vlCDR3 and scFv-CPA.9.049;

CPA.9.057, CPA.9.057.VH, CPA.9.057.VL, CPA.9.057.HC, CPA.9.057.LC, CPA.9.057.H1, CPA.9.057.H2, CPA.9.057.H3; CPA.9.057.H4; CPA.9.057.H4(S241P); CPA.9.057.vhCDR1, CPA.9.057.vhCDR2, CPA.9.057.vhCDR3, CPA.9.057.vlCDR1, CPA.9.057.vlCDR2, CPA.9.057.vlCDR3 and scFv-CPA.9.057;

CPA.9.059, CPA.9.059.VH, CPA.9.059.VL, CPA.9.059.HC, CPA.9.059.LC, CPA.9.059.H1, CPA.9.059.H2, CPA.9.059.H3; CPA.9.059.H4; CPA.9.059.H4(S241P);

CPA.9.059.vhCDR1, CPA.9.059.vhCDR2, CPA.9.059.vhCDR3, CPA.9.059.vlCDR1, CPA.9.059.vlCDR2, CPA.9.059.vlCDR3 and scFv-CPA.9.059;

CPA.9.083, CPA.9.083.VH, CPA.9.083.VL, CPA.9.083.HC, CPA.9.083.LC, CPA.9.083.H1, CPA.9.083.H2, CPA.9.083.H3; CPA.9.083.H4; CPA.9.083.H4(S241P); CPA.9.083.vhCDR1, CPA.9.083.vhCDR2, CPA.9.083.vhCDR3, CPA.9.083.vlCDR1, CPA.9.083.vlCDR2, CPA.9.083.vlCDR3 and scFv-CPA.9.083;

CPA.9.086, CPA.9.086.VH, CPA.9.086.VL, CPA.9.086.HC, CPA.9.086.LC, CPA.9.086.H1, CPA.9.086.H2, CPA.9.086.H3; CPA.9.086.H4; CPA.9.086.H4(S241P); CPA.9.086.vhCDR1, CPA.9.086.vhCDR2, CPA.9.086.vhCDR3, CPA.9.086.vlCDR1, CPA.9.086.vlCDR2, CPA.9.086.vlCDR3 and scFv-CPA.9.086;

CPA.9.089, CPA.9.089.VH, CPA.9.089.VL, CPA.9.089.HC, CPA.9.089.LC, CPA.9.089.H1, CPA.9.089.H2, CPA.9.089.H3; CPA.9.089.H4; CPA.9.089.H4(S241P); CPA.9.089.vhCDR1, CPA.9.089.vhCDR2, CPA.9.089.vhCDR3, CPA.9.089.vlCDR1, CPA.9.089.vlCDR2, CPA.9.089.vlCDR3 and scFv-CPA.9.089;

CPA.9.093, CPA.9.093.VH, CPA.9.093.VL, CPA.9.093.HC, CPA.9.093.LC, CPA.9.093.H1, CPA.9.093.H2, CPA.9.093.H3; CPA.9.093.H4; CPA.9.093.H4(S241P); CPA.9.093.vhCDR1, CPA.9.093.vhCDR2, CPA.9.093.vhCDR3, CPA.9.093.vlCDR1, CPA.9.093.vlCDR2, CPA.9.093.vlCDR3 and scFv-CPA.9.093;

CPA.9.101, CPA.9.101.VH, CPA.9.101.VL, CPA.9.101.HC, CPA.9.101.LC, CPA.9.101.H1, CPA.9.101.H2, CPA.9.101.H3; CPA.9.101.H4; CPA.9.101.H4(S241P); CPA.9.101.vhCDR1, CPA.9.101.vhCDR2, CPA.9.101.vhCDR3, CPA.9.101.vlCDR1, CPA.9.101.vlCDR2, CPA.9.101.vlCDR3 and scFv-CPA.9.101; and

CPA.9.103, CPA.9.103.VH, CPA.9.103.VL, CPA.9.103.HC, CPA.9.103.LC, CPA.9.103.H1, CPA.9.103.H2, CPA.9.103.H3; CPA.9.103.H4; CPA.9.103.H4(S241P); CPA.9.103.vhCDR1, CPA.9.103.vhCDR2, CPA.9.103.vhCDR3, CPA.9.103.vlCDR1, CPA.9.103.vlCDR2, CPA.9.103.vlCDR3 and scFv-CPA.9.103.

Furthermore, the present invention provides a number of CHA antibodies, which are murine antibodies generated from hybridomas. As is well known the art, the six CDRs are useful when put into either human framework variable heavy and variable light regions or when the variable heavy and light domains are humanized. Accordingly, the present invention provides antibodies, usually full length or scFv domains, that comprise the following sets of CDRs, the sequences of which are shown in FIG. 23A-23D and/or the sequence listing:

CHA.9.536.1, CHA.9.536.1.VH, CHA.9.536.1.VL, CHA.9.536.1.HC, CHA.9.536.1.LC, CHA.9.536.1.H1, CHA.9.536.1.H2, CHA.9.536.1.H3; CHA.9.536.1.H4, CHA.9.536.1.H4(S241P), CHA.9.536.1.vhCDR1, CHA.9.536.1.vhCDR2, CHA.9.536.1.vhCDR3, CHA.9.536.1.vlCDR1, CHA.9.536.1.vlCDR2 and CHA.9.536.1.vhCDR3;

CHA.9.536.3, CHA.9.536.3.VH, CHA.9.536.3.VL, CHA.9.536.3.HC, CHA.9.536.3.LC, CHA.9.536.3.H1, CHA.9.536.3.H2, CHA.9.536.3.H3; CHA.9.536.3.H4, CHA.9.536.3.H4(S241P); CHA.9.536.3.vhCDR1, CHA.9.536.3.vhCDR2, CHA.9.536.3.vhCDR3, CHA.9.536.3.vlCDR1, CHA.9.536.3.vlCDR2 and CHA.9.536.3.vhCDR3;

CHA.9.536.4, CHA.9.536.4.VH, CHA.9.536.4.VL, CHA.9.536.4.HC, CHA.9.536.4.LC, CHA.9.536.4.H1, CHA.9.536.4.H2, CHA.9.536.4.H3; CHA.9.536.4.H4, CHA.9.536.4.H4(S241P), CHA.9.536.4.vhCDR1, CHA.9.536.4.vhCDR2, CHA.9.536.4.vhCDR3, CHA.9.536.4.vlCDR1, CHA.9.536.4.vlCDR2 and CHA.9.536.4.vhCDR3;

CHA.9.536.5, CHA.9.536.5.VH, CHA.9.536.5.VL, CHA.9.536.5.HC, CHA.9.536.5.LC, CHA.9.536.5.H1, CHA.9.536.5.H2, CHA.9.536.5.H3; CHA.9.536.5.H4, CHA.9.536.5.H4(S241P), CHA.9.536.5.vhCDR1, CHA.9.536.5.vhCDR2, CHA.9.536.5.vhCDR3, CHA.9.536.5.vlCDR1, CHA.9.536.5.vlCDR2 and CHA.9.536.5.vhCDR3;

CHA.9.536.6, CHA.9.536.6.VH, CHA.9.536.6.VL, CHA.9.536.6.HC, CHA.9.536.6.LC, CHA.9.536.6.H1, CHA.9.536.6.H2, CHA.9.536.6.H3; CHA.9.536.6.H4, CHA.9.536.6.vhCDR1, CHA.9.536.6.vhCDR2, CHA.9.536.6.vhCDR3, CHA.9.536.6.vlCDR1, CHA.9.536.6.vlCDR2 and CHA.9.536.6.vhCDR3;

CHA.9.536.7, CHA.9.536.7.VH, CHA.9.536.7.VL, CHA.9.536.7.HC, CHA.9.536.7.LC, CHA.9.536.7.H1, CHA.9.536.7.H2, CHA.9.536.7.H3; CHA.9.536.7.H4, CHA.9.536.5.H4(S241P); CHA.9.536.7.vhCDR1, CHA.9.536.7.vhCDR2, CHA.9.536.7.vhCDR3, CHA.9.536.7.vlCDR1, CHA.9.536.7.vlCDR2 and CHA.9.536.7.vhCDR3;

CHA.9.536.8, CHA.9.536.8.VH, CHA.9.536.8.VL, CHA.9.536.8.HC, CHA.9.536.8.LC, CHA.9.536.8.H1, CHA.9.536.8.H2, CHA.9.536.8.H3; CHA.9.536.8.H4, CHA.9.536.8.H4(S241P), CHA.9.536.8.vhCDR1, CHA.9.536.8.vhCDR2, CHA.9.536.8.vhCDR3, CHA.9.536.8.vlCDR1, CHA.9.536.8.vlCDR2 and CHA.9.536.8.vhCDR3;

CHA.9.560.1, CHA.9.560.1VH, CHA.9.560.1.VL, CHA.9.560.1.HC, CHA.9.560.1.LC, CHA.9.560.1.H1, CHA.9.560.1.H2, CHA.9.560.1.H3; CHA.9.560.1.H4, CHA.9.560.1.H4(S241P), CHA.9.560.1.vhCDR1, CHA.9.560.1.vhCDR2, CHA.9.560.1.vhCDR3, CHA.9.560.1.vlCDR1, CHA.9.560.1.vlCDR2 and CHA.9.560.1.vhCDR3;

CHA.9.560.3, CHA.9.560.3VH, CHA.9.560.3.VL, CHA.9.560.3.HC, CHA.9.560.3.LC, CHA.9.560.3.H1, CHA.9.560.3.H2, CHA.9.560.3.H3; CHA.9.560.3.H4, CHA.9.560.3.H4(S241P); CHA.9.560. 3.vhCDR1, CHA.9.560. 3.vhCDR2, CHA.9.560.3.vhCDR3, CHA.9.560.3.vlCDR1, CHA.9.560.3.vlCDR2 and CHA.9.560.3.vhCDR3;

CHA.9.560.4, CHA.9.560.4VH, CHA.9.560.4.VL, CHA.9.560.4.HC, CHA.9.560.4.LC, CHA.9.560.4.H1, CHA.9.560.4.H2, CHA.9.560.4.H3; CHA.9.560.4.H4, CHA.9.560.4.H4(S241P), CHA.9.560. 4.vhCDR1, CHA.9.560. 4.vhCDR2, CHA.9.560.4.vhCDR3, CHA.9.560.4.vlCDR1, CHA.9.560.4.vlCDR2 and CHA.9.560.4.vhCDR3;

CHA.9.560.5, CHA.9.560.5VH, CHA.9.560. 5.VL, CHA.9.560. 5.HC, CHA.9.560.5.LC, CHA.9.560.5.H1, CHA.9.560.5.H2, CHA.9.560.5.H3; CHA.9.560. 5.H4, CHA.9.560.5.vhCDR1, CHA.9.560.5.vhCDR2, CHA.9.560.5.vhCDR3, CHA.9.560.5.vlCDR1, CHA.9.560.5.vlCDR2 and CHA.9.560.5.vhCDR3;

CHA.9.560.6, CHA.9.560.6VH, CHA.9.560.6.VL, CHA.9.560. 6.HC, CHA.9.560.6.LC, CHA.9.560.6.H1, CHA.9560.6.H2, CHA.9.560.6.H3; CHA.9.560.6.H4, CHA.9.560.6.H4(S241P), CHA.9.560.6.vhCDR1, CHA.9.560.6.vhCDR2, CHA.9.560.6.vhCDR3, CHA.9.560.6.vlCDR1, CHA.9.560.6.vlCDR2 and CHA.9.560.6.vhCDR3;

CHA.9.560.7, CHA.9.560.7VH, CHA.9.560.7.VL, CHA.9.560.7.HC, CHA.9.560.7.LC, CHA.9.560.7.H1, CHA.9.560.7.H2, CHA.9.560.7.H3; CHA.9.560.7.H4; CHA.9.560.7.H4(S241P); CHA.9.560.7.vhCDR1, CHA.9.560.7.vhCDR2, CHA.9.560.7.vhCDR3, CHA.9.560.7.vlCDR1, CHA.9.560.7.vlCDR2 and CHA.9.560.7.vhCDR3;

CHA.9.560.8, CHA.9.560.8VH, CHA.9.560.8.VL, CHA.9.560.8.HC, CHA.9.560.8.LC, CHA.9.560.8.H1, CHA.9.560.8.H2, CHA.9.560.8.H3; CHA.9.560.8.H4, CHA.9.560.8.H4(S241P); CHA.9.560.8.vhCDR1, CHA.9.560.8.vhCDR2, CHA.9.560.8.vhCDR3, CHA.9.560.8.vlCDR1, CHA.9.560.8.vlCDR2 and CHA.9.560.8.vhCDR3;

CHA.9.546.1, CHA.9.546.1VH, CHA.9.546.1.VL, CHA.9.546.1.HC, CHA.9.546.1.LC, CHA.9.546.1.H1, CHA.9.546.1.H2, CHA.9.546.1.H3; CHA.9.546.1.H4, CHA.9.546.1.H4(S241P), CHA.9.546.1.vhCDR1, CHA.9.546.1.vhCDR2, CHA.9.546.1.vhCDR3, CHA.9.546.1.vlCDR1, CHA.9.546.1.vlCDR2 and CHA.9.546.1.vhCDR3;

CHA.9.547.1, CHA.9.547.1VH, CHA.9.547.1.VL, CHA.9.547.1.HC, CHA.9.547.1.LC, CHA.9.547.1.H1, CHA.9.547.1.H2, CHA.9.547.1.H3; CHA.9.547.1.H4, CHA.9.547.1.H4(S241P), CHA.9.547.1.vhCDR1, CHA.9.547.1.vhCDR2, CHA.9.547.1.vhCDR3, CHA.9.547.1.vlCDR1, CHA.9.547.1.vlCDR2 and CHA.9.547.1.vhCDR3;

CHA.9.547.2, CHA.9.547.2VH, CHA.9.547.2.VL, CHA.9.547.2.HC, CHA.9.547.2.LC, CHA.9.547.2.H1, CHA.9.547.2.H2, CHA.9.547.2.H3; CHA.9.547.2.H4, CHA.9.547.2.H4(S241P), CHA.9.547.2.vhCDR1, CHA.9.547.2.vhCDR2, CHA.9.547. 2.vhCDR3, CHA.9.547.2.vlCDR1, CHA.9.547.2.vlCDR2 and CHA.9.547.2.vhCDR3;

CHA.9.547.3, CHA.9.547. 3VH, CHA.9.547. 3.VL, CHA.9.547. 3.HC, CHA.9.547. 3.LC, CHA.9.547. 3.H1, CHA.9.547. 3.H2, CHA.9.547. 3.H3; CHA.9.547.3.H4, CHA.9.547.3.H4(S241P), CHA.9.547. 3.vhCDR1, CHA.9.547. 3.vhCDR2, CHA.9.547. 3.vhCDR3, CHA.9.547. 3.vlCDR1, CHA.9.547. 3.vlCDR2 and CHA.9.547. 3.vhCDR3;

CHA.9.547.4, CHA.9.547. 4VH, CHA.9.547. 4.VL, CHA.9.547. 4.HC, CHA. 9.547. 4.LC, CHA.9.547. 4.H1, CHA.9.547. 4.H2, CHA.9.547. 4.H3; CHA.9.547.4.H4, CHA.9.547.4.H4(S241P), CHA.9.547. 4.vhCDR1, CHA.9.547. 4.vhCDR2, CHA.9.547. 4.vhCDR3, CHA.9.547. 4.vlCDR1, CHA.9.547. 4.vlCDR2 and CHA.9.547. 4.vhCDR3;

CHA.9.547.6, CHA.9.547. 6 VH, CHA.9.547. 6.VL, CHA.9.547. 6.HC, CHA.9.547. 6.LC, CHA.9.547. 6.H1, CHA.9.547. 6.H2, CHA.9.547. 6.H3; CHA.9.547.6.H4, CHA.9.547.6.H4(S241P), CHA.9.547. 6.vhCDR1, CHA.9.547. 6.vhCDR2, CHA.9.547. 6.vhCDR3, CHA.9.547. 6.vlCDR1, CHA.9.547. 6.vlCDR2 and CHA.9.547. 6.vhCDR3;

CHA.9.547.7, CHA.9.547. 7VH, CHA.9.547. 7.VL, CHA.9.547. 7.HC, CHA.9.547. 7.LC, CHA.9.547. 7.H1, CHA.9.547. 7.H2, CHA.9.547. 7.H3; CHA.9.547.7.H4, CHA.9.547.7.H4(S241P), CHA.9.547. 7.vhCDR1, CHA.9.547. 7.vhCDR2, CHA.9.547. 7.vhCDR3, CHA.9.547. 7.vlCDR1, CHA.9.547. 7.vlCDR2 and CHA.9.547. 7.vhCDR3;

CHA.9.547.8, CHA.9.547. 8VH, CHA.9.547. 8.VL, CHA.9.547. 8.HC, CHA.9.547.8.LC, CHA.9.547. 8.H1, CHA.9.547. 8.H2, CHA.9.547. 8.H3; CHA.9.547.8.H4, CHA.9.547.8.H4(S241P), CHA.9.547. 8.vhCDR1, CHA.9.547. 8.vhCDR2, CHA.9.547. 8.vhCDR3, CHA.9.547. 8.vlCDR1, CHA.9.547. 8.vlCDR2 and CHA.9.547. 8.vhCDR3;

CHA.9.547.9, CHA.9.547.9, CHA.9.547.9VH, CHA.9.547.9.VL, CHA.9. 547.9.HC, CHA.9.547.9.LC, CHA.9.547.9.H1, CHA.9.547.9.H2, CHA.9.547.9.H3; CHA.9.547.9.H4, CHA.9.547.9.H4, CHA.9.547.9.H4(S241P), CHA.9.547.9.H4(S241P), CHA.9.547.9.vhCDR1, CHA.9.547.9.vhCDR2, CHA.9.547.9.vhCDR3, CHA.9.547.9.vlCDR1, CHA.9.547.9.vlCDR2 and CHA.9.547.9.vhCDR3;

CHA.9.547.13, CHA.9.547.13, CHA.9.547. 13VH, CHA.9. 547.13.VL, CHA.9. 547.13.HC, CHA.9.547.13.LC, CHA.9.547.13.H1, CHA.9.547.13.H2, CHA.9. 547.13.H3; CHA.9.547.13.H4, CHA.9.547.13.H4, CHA.9.547.13.H4(S241P), CHA.9.547.13.H4(S241P), CHA.9.547.13.vhCDR1, CHA.9.547.13.vhCDR2, CHA.9.547. 13.vhCDR3, CHA.9.547.13.vlCDR1, CHA.9.547.13.vlCDR2 and CHA.9.547. 13.vhCDR3;

CHA.9.541.1, CHA.9.541.1.VH, CHA.9.541.1.VL, CHA.9.541.1.HC, CHA.9.541.1.LC, CHA.9.541.1.H1, CHA.9.541.1.H2, CHA.9.541.1.H3; CHA.9.541.1.H4, CHA.9.541.1.H4(S241P), CHA.9.541.1.vhCDR1, CHA.9.541.1.vhCDR2, CHA.9.541.1.vhCDR3, CHA.9.541.1.vlCDR1, CHA.9.541.1.vlCDR2 and CHA. 9.541.1.vhCDR3;

CHA.9.541.3, CHA.9.541. 3.VH, CHA.9.541. 3.VL, CHA.9.541. 3.HC, CHA.9.541. 3.LC, CHA.9.541. 3.H1, CHA.9.541. 3.H2, CHA.9.541. 3.H3; CHA.9.541.3.H4, CHA.9.541.3.H4(S241P), CHA.9.541. 3.vhCDR1, CHA.9.541. 3.vhCDR2, CHA.9.541. 3.vhCDR3, CHA.9.541. 3.vlCDR1, CHA.9.541. 3.vlCDR2 and CHA.9.541. 3.vhCDR3;

CHA.9.541.4, CHA.9.541.4.VH, CHA.9.541. 4.VL, CHA.9.541. 4.HC, CHA.9.541. 4.LC, CHA.9.541. 4.H1, CHA.9.541. 4.H2, CHA.9.541. 4.H3; CHA.9.541.4.H4, CHA.9.541.4.H4(S241P), CHA.9.541. 4.vhCDR1, CHA.9.541. 4.vhCDR2, CHA.9.541. 4.vhCDR3, CHA.9.541. 4.vlCDR1, CHA.9.541. 4.vlCDR2 and CHA.9.541. 4.vhCDR3;

CHA.9.541.5, CHA.9.541. 5.VH, CHA.9.541. 5.VL, CHA.9.541. 5.HC, CHA.9.541. 5.LC, CHA.9.541. 5.H1, CHA.9.541. 5.H2, CHA.9.541. 5.H3; CHA.9.541.5.H4, CHA.9.541.5.H4(S241P), CHA.9.541. 5.vhCDR1, CHA.9.541. 5.vhCDR2, CHA.9.541. 5.vhCDR3, CHA.9.541. 5.vlCDR1, CHA.9.541. 5.vlCDR2 and CHA.9.541. 5.vhCDR3;

CHA.9.541.6, CHA.9.541. 6.VH, CHA.9.541. 6.VL, CHA.9.541. 6.HC, CHA.9.541. 6.LC, CHA.9.541. 6.H1, CHA.9.541. 6.H2, CHA.9.541.6.H3; CHA.9.541.6.H4, CHA.9.541.6.H4(S241P), CHA.9.541. 6.vhCDR1, CHA.9.541. 6.vhCDR2, CHA.9.541. 6.vhCDR3, CHA.9.541. 6.vlCDR1, CHA.9.541. 6.vlCDR2 and CHA.9.541. 6.vhCDR3;

CHA.9.541.7, CHA.9.541. 7.VH, CHA.9.541. 7.VL, CHA.9.541. 7.HC, CHA.9.541. 7.LC, CHA.9.541. 7.H1, CHA.9.541. 7.H2, CHA.9.541. 7.H3; CHA.9.541.7.H4, CHA.9.541.7.H4(S241P), CHA.9.541. 7.vhCDR1, CHA.9.541. 7.vhCDR2, CHA.9.541. 7.vhCDR3, CHA.9.541. 7.vlCDR1, CHA.9.541. 7.vlCDR2 and CHA.9.541. 7.vhCDR3; and

CHA.9.541.8, CHA.9.541. 8.VH, CHA.9.541. 8.VL, CHA.9.541. 8.HC, CHA.9.541. 8.LC, CHA.9.541. 8.H1, CHA.9.541. 8.H2, CHA.9.541. 8.H3; CHA.9.541.8.H4, CHA.9.541.8.H4(S241P); CHA.9.541. 8vhCDR1, CHA.9.541. 8.vhCDR2, CHA.9.541. 8.vhCDR3, CHA.9.541. 8.vlCDR1, CHA.9.541. 8.vlCDR2 and CHA.9.541. 8.vhCDR3.

CHA.9.547.18vhCDR1, CHA.9.547.18.vhCDR2, CHA.9.547.18.vhCDR3, CHA.9.547.18.vlCDR1, CHA.9.547.18vlCDR2, and CHA.9.547.18.vlCDR3.

In the case of scFvs comprising the CDRs of the antibodies above, these are labeled as scFvs that include a scFv comprising a variable heavy domain with the vhCDRs, a linker and a variable light domain with the vlCDRs, again as above in either orientation. Thus the invention includes scFv-CHA.9.536.3.1, scFv-CHA.9.536.3, scFv-CHA.9.536.4, scFv-CHA.9.536.5, scFv-CHA.9.536.7, scFv-CHA.9.536.8, scFv-CHA.9.560.1, scFv-CHA.9.560.3, scFv-CHA.9.560.4, scFv-CHA.9.560.5, scFv-CHA.9.560.6, scFv-CHA.9.560.7, scFv-CHA.9.560.8, scFv-CHA.9.546.1, scFv-CHA.9.547.1, scFv-CHA.9.547.2, scFv-CHA.9.547.3, scFv-CHA.9.547.4, scFv-CHA.9.547.6, scFv-CHA.9.547.7, scFv-CHA.9.547.8, scFv-CHA.9.547.9, scFv-CHA.9.547.13, scFv-CHA.9.541.1, scFv-CHA.9.541.3, scFv-CHA.9.541.4, scFv-CHA.9.541.5, scFv-CHA.9.541.6, scFv-CHA.9.541.7 and scFv-CHA.9.541.8.

In addition, CHA.9.543 binds to TIGIT but does not block the TIGIT-PVR interaction.

As discussed herein, the invention further provides variants of the above components (CPA and CHA), including variants in the CDRs, as outlined above. Thus, the invention provides antibodies comprising a set of 6 CDRs as outlined herein that can contain one, two or three amino acid differences in the set of CDRs, as long as the antibody still binds to TIGIT. Suitable assays for testing whether an anti-PVRIG/anti-TIGIT bispecific antibody that contains mutations as compared to the CDR sequences outlined herein are known in the art, such as Biacore assays.

In addition, the invention further provides variants of the above variable heavy and light chains. In this case, the variable heavy chains can be 80%, 90%, 95%, 98% or 99% identical to the “VH” sequences herein, and/or contain from 1, 2, 3, 4, 5, 6, 7, 8, 9, 10 amino acid changes, or more, when Fc variants are used. Variable light chains are provided that can be 80%, 90%, 95%, 98% or 99% identical to the “VL” sequences herein (and in particular CPA.9.086), and/or contain from 1, 2, 3, 4, 5, 6, 7, 8, 9, 10 amino acid changes, or more, when Fc variants are used. In these embodiments, the invention includes these variants as long as the anti-PVRIG/anti-TIGIT bispecific antibody still binds to TIGIT. Suitable assays for testing whether an anti-TIGIT antibody that contains mutations as compared to the CDR sequences outlined herein are known in the art, such as Biacore assays.

Similarly, heavy and light chains are provided that are 80%, 90%, 95%, 98% or 99% identical to the full length “HC” and “LC” sequences herein (and in particular CPA.9.086), and/or contain from 1, 2, 3, 4, 5, 6, 7, 8, 9, 10 amino acid changes, or more, when Fc variants are used. In these embodiments, the invention includes these variants as long as the anti-PVRIG/anti-TIGIT bispecific antibody still binds to TIGIT. Suitable assays for testing whether an anti-PVRIG/anti-TIGIT bispecific antibody that contains mutations as compared to the CDR sequences outlined herein are known in the art, such as Biacore assays.

In addition, the framework regions of the variable heavy and variable light chains of either the CPA or CHA antibodies herein can be humanized (or, in the case of the CHA antibodies, “rehumanized”, to the extent that alternative humanization methods can be done) as is known in the art (with occasional variants generated in the CDRs as needed), and thus humanized variants of the VH and VL chains of FIG. 23A-23D can be generated (and in particular CPA.9.086). Furthermore, the humanized variable heavy and light domains can then be fused with human constant regions, such as the constant regions from IgG1, IgG2, IgG3 and IgG4 (including IgG4(S241P)).

In particular, as is known in the art, murine VH and VL chains can be humanized as is known in the art, for example, using the IgBLAST program of the NCBI website, as outlined in Ye et al. Nucleic Acids Res. 41:W34-W40 (2013), herein incorporated by reference in its entirety for the humanization methods. IgBLAST takes a murine VH and/or VL sequence and compares it to a library of known human germline sequences. As shown herein, for the humanized sequences generated herein, the databases used were IMGT human VH genes (F+ORF, 273 germline sequences) and IMGT human VL kappa genes (F+ORF, 74 germline sequences). An exemplary five CHA sequences were chosen: CHA.9.536, CHA9.560, CHA.9.546, CHA.9.547 and CHA.9.541 (see FIG. 23A-23D). For this embodiment of the humanization, human germline IGHV1-46(allele1) was chosen for all 5 as the acceptor sequence and the human heavy chain IGHJ4(allele1) joining region (J gene). For three of four (CHA.7.518, CHA.7.530, CHA.7.538_1 and CHA.7.538_2), human germline IGKV1-39(allele 1) was chosen as the acceptor sequence and human light chain IGKJ2(allele1) (J gene) was chosen. The J gene was chosen from human joining region sequences compiled at IMGT® the international ImMunoGeneTics information system as www.imgt.org. CDRs were defined according to the AbM definition (see www.bioinfo.org.uk/abs/). In some embodiments, the anti-PVRIG/anti-TIGIT bispecific antibodies of the present invention include TIGIT binding portions or antigen binding domainswherein the V_{H} and V_{L} sequences of different TIGIT binding portions or antigen binding domains can be “mixed and matched” to create other TIGIT binding portions or antigen binding domains. TIGIT binding of such “mixed and matched” anti-PVRIG/anti-TIGIT bispecific antibodies can be tested using the binding assays described above. e.g., ELISAs or Biacore assays). In some embodiments, when V_{H} and V_{L} chains are mixed and matched, a V_{H} sequence from a particular V_{H}/V_{L} pairing is replaced with a structurally similar V_{H} sequence. Likewise, in some embodiments, a V_{L} sequence from a particular V_{H}/V_{L} pairing is replaced with a structurally similar V_{L} sequence. For example, the V_{H} and V_{L} sequences of homologous antibodies are particularly amenable for mixing and matching.

Accordingly, the anti-PVRIG/anti-TIGIT bispecific antibodies of the invention comprise CDR amino acid sequences selected from the group consisting of (a) sequences as listed herein; (b) sequences that differ from those CDR amino acid sequences specified in (a) by 1, 2, 3, 4, 5, 6, 7, 8, 9, 10 or more amino acid substitutions; (c) amino acid sequences having 90% or greater, 95% or greater, 98% or greater, or 99% or greater sequence identity to the sequences specified in (a) or (b); (d) a polypeptide having an amino acid sequence encoded by a polynucleotide having a nucleic acid sequence encoding the amino acids as listed herein. In particular, the anti-PVRIG/anti-TIGIT bispecific antibody can comprise the antigen bidng domain from the the CPA.9.086 antibody which can have sequences selected from (a), (b), (c) or (d).

In some embodiments, the anti-PVRIG/anti-TIGIT bispecific antibody comprises the following sequences (FIG. 26A-26B; amino acid substitutions for the knob into hole heterodimerization approach are bold and underlined):

CHA.7.518.1.H4(S241P) VH IgG1 CH1 IgG4 CH2—CH3 S354C, E356D, M358L,
T366W
(SEQ ID NO: 3175)
QVQLVQSGAEVKKPGASVKVSCKASGYTFTDYNINWVRQAPGQGLEWMGYIYPYIGGSGYAQKFQGRVT
MTRDTSTSTVYMELSSLRSEDTAVYYCAREDKTARNAMDYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISKAKGQPRE
PQVYTLPPCQDELTKNQVSLWCLVKGFYPSDIAVEWESNGQPENNYKTTPPVLDSDGSFFLYSRLTVDK
SRWQEGNVFSCSVMHEALHNHYTQKSLSLSPGK;
CHA.7.518.1.H4(S241P) VL Ckappa
(SEQ ID NO: 3176)
DIQMTQSPSSLSASVGDRVTITCRVSENIYSNLAWYQQKPGKAPKLLIYEATNLAEGVPSRFSGSGSGT
DFTLTISSLQPEDFATYYCQHFWGTPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGEC;
and
CPA.9.086 HC Clambda Crossmab IgG4 Y349C, E356D, M358L, T366S, L368A,
Y407V
(SEQ ID NO: 3177)
EVQLVETGGGLIQPGRSLRLSCAASGFTFSSYAMHWVRQAPGKGLEWVAVISYAGEVKYYADSVKGRFT
ISRDNSKNTLYLQMNSLRAEDTAVYYCARDPLPLHYYGMDVWGQGTTVTVSSASGQPKAAPSVTLFPPS
SEELQANKATLVCLISDFYPGAVTVAWKADSSPVKAGVETTTPSKQSNNKYAASSYLSLTPEQWKSHRS
YSCQVTHEGSTVEKTVAPTECSESKYGPPCPPCPAPEFLGGPSVFLFPPKPKDTLMISRTPEVTCVVVD
VSQEDPEVQFNWYVDGVEVHNAKTKPREEQFNSTYRVVSVLTVLHQDWLNGKEYKCKVSNKGLPSSIEK
TISKAKGQPREPQVCTLPPSQDELTKNQVSLSCAVKGFYPSDIAVEWESNGQPENNYKTTPPVLDSDGS
FFLVSRLTVDKSRWQEGNVFSCSVMHEALHNHYTQKSLSLSPGK;
CPA.9.086 LC CH1
(SEQ ID NO: 3178)
QSALTQPRSASGNPGQRVTISCSGSSSNMGRRPVNWYQQIPGTAPKLLIYSQNQRPSGVPDRFSGSQSG
TSASLTISGLQSEDEAEYFCAVWDDIGRVLQLGGGTQLAVLSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVD
KKVEPKSC.

In some embodiments, the anti-PVRIG/anti-TIGIT bispecific antibody comprises the following sequences:

CHA.7.518.1 VH

(SEQ ID NO: 1539)

QVQLVQSGAEVKKPGASVKVSCKASGYTFTDYNINWVRQAPGQGLEWM
GYIYPYIGGSGYAQKFQGRVTMTRDTSTSTVYMELSSLRSEDTAVYYCA
REDKTARNAMDYWGQGTLVTVSS;
CHA.7.518.1 VL

(SEQ ID NO: 1544)

DIQMTQSPSSLSASVGDRVTITCRVSENIYSNLAWYQQKPGKAPKLLIY
EATNLAEGVPSRFSGSGSGTDFTLTISSLQPEDFATYYCQHFWGTPYT
FGQGTKLEIK;
and
CPA.9.086 VH

(SEQ ID NO: 1634)

EVQLVETGGGLIQPGRSLRLSCAASGFTFSSYAMHWVRQAPGKGLEWV
AVISYAGEVKYYADSVKGRFTISRDNSKNTLYLQMNSLRAEDTAVYYCA
RDPLPLHYYGMDVWGQGTTVTVSS;
CPA.9.086 VL

(SEQ ID NO: 1639)

QSALTQPRSASGNPGQRVTISCSGSSSNMGRRPVNWYQQIPGTAPKLLI
YSQNQRPSGVPDRFSGSQSGTSASLTISGLQSEDEAEYFCAVWDDIGR
VLQLGGGTQLAVL.

In some embodiments, the anti-PVRIG/anti-TIGIT bispecific antibody comprises the following sequences:

CHA.7.518.4 VH

(SEQ ID NO: 3179)

QVQLVQSGAEVKKPGASVKVSCKASGYTFTDYNINWVRQAPGQGLEWM
GYIYPYIGGSGYAQKFQGRVTMTRDTSTSTVYMELSSLRSEDTAVYYCA
REDKTARNAMDYWGQGTLVTVSS;
CHA.7.518.4 VL

(SEQ ID NO: 3180)

DIQMTQSPSSLSASVGDRVTITCRVSENIYDVLAWYQQKPGKAPKLLIY
EATNLAEGVPSRFSGSGSGTDFTLTISSLQPEDFATYYCQHFWGTPYT
FGQGTKLEIK;
and
CPA.9.086 VH

(SEQ ID NO: 1634)

EVQLVETGGGLIQPGRSLRLSCAASGFTFSSYAMHWVRQAPGKGLEWVA
VISYAGEVKYYADSVKGRFTISRDNSKNTLYLQMNSLRAEDTAVYYCAR
DPLPLHYYGMDVWGQGTTVTVSS;
CPA.9.086 VL

(SEQ ID NO: 1639)

QSALTQPRSASGNPGQRVTISCSGSSSNMGRRPVNWYQQIPGTAPKLL
IYSQNQRPSGVPDRFSGSQSGTSASLTISGLQSEDEAEYFCAVWDDIGR
VLQLGGGTQLAVL.

In some embodiments, the anti-PVRIG/anti-TIGIT bispecific antibody comprises the following sequences:

CHA.7.518.1 VH

(SEQ ID NO: 1539)

QVQLVQSGAEVKKPGASVKVSCKASGYTFTDYNINWVRQAPGQGLEWM
GYIYPYIGGSGYAQKFQGRVTMTRDTSTSTVYMELSSLRSEDTAVYYCA
REDKTARNAMDYWGQGTLVTVSS;
CHA.7.518.1 VL

(SEQ ID NO: 1544)

DIQMTQSPSSLSASVGDRVTITCRVSENIYSNLAWYQQKPGKAPKLLIY
EATNLAEGVPSRFSGSGSGTDFTLTISSLQPEDFATYYCQHFWGTPYT
FGQGTKLEIK;
and
CHA.9.547.18 VH

(SEQ ID NO: 1664)

EVQLVESGGGLVQPGGSLRLSCAASGFTFSSYIMSWVRQAPGKGLEWVA
TISGGSKGQYYADSVKGRFTISRDNSKNTLYLQMNSLRAEDTAVYYCAK
WLLSYYAMDYWGQGTLVTVSS;
CHA.9.547.18 VL

(SEQ ID NO: 1668)

DIQMTQSPSSLSASVGDRITITCRASQSMAIWLSWYQQKPGKAPKLLIY
KASKSHTGVPSRFSGSGSGTDFTLTISSLQPEDFATYYCQQGQSYPYTF
GQGTKLEIK.

In some embodiments, the anti-PVRIG/anti-TIGIT bispecific antibody comprises the following sequences:

CHA.7.518.4 VH

(SEQ ID NO: 3179)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.4 VL

(SEQ ID NO: 3180)

DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIK;
and
CHA.9.547.18 VH

(SEQ ID NO: 1664)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSS;
CHA.9.547.18 VL

(SEQ ID NO: 1668)

DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIK.

In some embodiments, the anti-PVRIG portion of the anti-PVRIG/anti-TIGIT bispecific antibody comprises one of the following anti-PVRIG sequences. ( ) in the sequences below indicates a deletion relative to the reference human IgG4 amino acid sequence. Bolded text indicates amino acid substitutions relative to the reference IgG4 sequence.

In some embodiments, the anti-PVRIG portion of the anti-PVRIG/anti-TIGIT bispecific antibody comprises one of the following sequences:

CHA.7.518.1 scFv (VL-linker-VH) hIgG4 Variant 1
(SEQ ID NO: 3217)

DIQMTQSPSSLSASVGDRVTITCRVSENIYSNLAWYQQKPGKAPKLLIYEATNLAEGVPSRFSGSGSGTDFT
LTISSLQPEDFATYYCQHFWGTPYTFGQGTKLEIKggggsggggsggggsQVQLVQSGAEVKKPGASVKVSC
KASGYTFTDYNINWVRQAPGQGLEWMGYIYPYIGGSGYAQKFQGRVTMTRDTSTSTVYMELSSLRSEDTAVY
YCAREDKTARNAMDYWGQGTLVTVSSESKYGPPCPPCPAPPVA()GPSVFLFPPKPKDTLMISRTPEVTCVV
VDVKQEDPEVQFNWYVDGVEVHNAKTKPREEQFNSTYRVVSVLTVLHQDWLNGKEYKCKVSNKGLPSSIEKT
ISKAKGQPREPQVYTLPPSQEQMTKNQVKLTCLVKGFYPSDIAVEWESNGQPENNYKTTPPVLDSDGSFFLY
SRLTVDKSRWQEGNVFSCSVMHEALHNHYTQKSLSLSPG;
CHA.7.518.1 scFv (VH-linker-VL) hIgG4 Variant 1

(SEQ ID NO: 3227)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSggggsggggsggggsDIQMTQSPS
SLSASVGDRVTITCRVSENIYSNLAWYQQKPGKAPKLLIYEATNLAEGVPSRFSGSGSGTDFTLTISSLQPE
DFATYYCQHFWGTPYTFGQGTKLEIKESKYGPPCPPCPAPPVA()GPSVFLFPPKPKDTLMISRTPEVTCVV
VDVKQEDPEVQFNWYVDGVEVHNAKTKPREEQFNSTYRVVSVLTVLHQDWLNGKEYKCKVSNKGLPSSIEKT
ISKAKGQPREPQVYTLPPSQEQMTKNQVKLTCLVKGFYPSDIAVEWESNGQPENNYKTTPPVLDSDGSFFLY
SRLTVDKSRWQEGNVFSCSVMHEALHNHYTQKSLSLSPG;
CHA.7.518.1 scFv (VL-linker-VH) hIgG4 Variant 2
(SEQ ID NO: 3237)
DIQMTQSPSSLSASVGDRVTITCRVSENIYSNLAWYQQKPGKAPKLLIYEATNLAEGVPSRFSGSGSGTDFT
LTISSLQPEDFATYYCQHFWGTPYTFGQGTKLEIKggggsggggsggggsQVQLVQSGAEVKKPGASVKVSC
KASGYTFTDYNINWVRQAPGQGLEWMGYIYPYIGGSGYAQKFQGRVTMTRDTSTSTVYMELSSLRSEDTAVY
YCAREDKTARNAMDYWGQGTLVTVSSESKYGPPCPPCPAPPVA()GPSVFLFPPKPKDTLMISRTPEVTCVV
VDVKQEDPEVQFNWYVDGVEVHNAKTKPREEEFNSTYRVVSVLTVLHQDWLNGKEYKCKVSNKGLPSSIEKT
ISKAKGQPREPQVYTLPPSQEEMTKNQVSLTCDVSGFYPSDIAVEWESDGQPENNYKTTPPVLDSDGSFFLY
SRLTVDKSRWEEGDVFSCSVMHEALHNHYTQKSLSLSPG;
CHA.7.518.1 scFv (VH-linker-VL) hIgG4 Variant 2
(SEQ ID NO: 3247)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSggggsggggsggggsDIQMTQSPS
SLSASVGDRVTITCRVSENIYSNLAWYQQKPGKAPKLLIYEATNLAEGVPSRFSGSGSGTDFTLTISSLQPE
DFATYYCQHFWGTPYTFGQGTKLEIKESKYGPPCPPCPAPPVA()GPSVFLFPPKPKDTLMISRTPEVTCVV
VDVKQEDPEVQFNWYVDGVEVHNAKTKPREEEFNSTYRVVSVLTVLHQDWLNGKEYKCKVSNKGLPSSIEKT
ISKAKGQPREPQVYTLPPSQEEMTKNQVSLTCDVSGFYPSDIAVEWESDGQPENNYKTTPPVLDSDGSFFLY
SRLTVDKSRWEEGDVFSCSVMHEALHNHYTQKSLSLSPG;;
CHA.7.518.1 hIgG4 Variant 1
(SEQ ID NO: 3257)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSASTKGPSVFPLAPCSRSTSESTAA
LGCLVKDYFPEPVTVSWNSGALTSGVHTFPAVLQSSGLYSLSSVVTVPSSSLGTKTYTCNVDHKPSNTKVDK
RVESKYGPPCPPCPAPPVA()GPSVFLFPPKPKDTLMISRTPEVTCVVVDVKQEDPEVQFNWYVDGVEVHNA
KTKPREEQFNSTYRVVSVLTVLHQDWLNGKEYKCKVSNKGLPSSIEKTISKAKGQPREPQVYTLPPSQEQMT
KNQVKLTCLVKGFYPSDIAVEWESNGQPENNYKTTPPVLDSDGSFFLYSRLTVDKSRWQEGNVFSCSVMHEA
LHNHYTQKSLSLSPG;;
CHA.7.518.1 hIgG4 Variant 2
(SEQ ID NO: 3267)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSASTKGPSVFPLAPCSRSTSESTAA
LGCLVKDYFPEPVTVSWNSGALTSGVHTFPAVLQSSGLYSLSSVVTVPSSSLGTKTYTCNVDHKPSDTKVDK
RVESKYGPPCPPCPAPPVA()GPSVFLFPPKPKDTLMISRTPEVTCVVVDVKQEDPEVQFNWYVDGVEVHNA
KTKPREEEFNSTYRVVSVLTVLHQDWLNGKEYKCKVSNKGLPSSIEKTISKAKGQPREPQVYTLPPSQEEMT
KNQVSLTCDVSGFYPSDIAVEWESDGQPENNYKTTPPVLDSDGSFFLYSRLTVDKSRWEEGDVFSCSVMHEA
LHNHYTQKSLSLSPG;;
CHA.7.518.1 VH Clambda-VL-CH1 Crossmab hIgG4 Fc Y349C E356D L358L T366S
L368A Y407V Hole
(SEQ ID NO: 3277)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSASVAAPSVFIFPPSDEQLKSGTAS
VVCLLNNFYPREAKVQWKVDNALQSGNSQESVTEQDSKDSTYSLSSTLTLSKADYEKHKVYACEVTHQGLSS
PVTKSFNRGECESKYGPPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQVCTL
PPSQDELTKNQVSLSCAVKGFYPSDIAVEWESNGQPENNYKTTPPVLDSDGSFFLVSRLTVDKSRWQEGNVF
SCSVMHEALHNHYTQKSLSLSPG;;
CHA.7.518.1 VH Clambda-VL-CH1 Crossmab hIgG4 Fc Y354C E356D M348L T366W
Knob
(SEQ ID NO: 3287)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSASVAAPSVFIFPPSDEQLKSGTAS
VVCLLNNFYPREAKVQWKVDNALQSGNSQESVTEQDSKDSTYSLSSTLTLSKADYEKHKVYACEVTHQGLSS
PVTKSFNRGECESKYGPPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQVYTL
PPCQDELTKNQVSLWCLVKGFYPSDIAVEWESNGQPENNYKTTPPVLDSDGSFFLYSRLTVDKSRWQEGNVF
SCSVMHEALHNHYTQKSLSLSPG;;
CHA.7.518.1 VH hIgG4 Fc Y349C E356D L358L T366S L368A Y407V Hole
(SEQ ID NO: 3297)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSASASTKGPSVFPLAPCSRSTSEST
AALGCLVKDYFPEPVTVSWNSGALTSGVHTFPAVLQSSGLYSLSSVVTVPSSSLGTKTYTCNVDHKPSNTKV
DKRVESKYGPPCPPCPAPEFLGGPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTISKAKGQPREPQVCTLPPSQDEL
TKNQVSLSCAVKGFYPSDIAVEWESNGQPENNYKTTPPVLDSDGSFFLVSRLTVDKSRWQEGNVFSCSVMHE
ALHNHYTQKSLSLSPG;;
CHA.7.518.1 VH hIgG4 Fc Y354C E356D M348L T366W Knob
(SEQ ID NO: 3307)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSASASTKGPSVFPLAPCSRSTSEST
AALGCLVKDYFPEPVTVSWNSGALTSGVHTFPAVLQSSGLYSLSSVVTVPSSSLGTKTYTCNVDHKPSNTKV
DKRVESKYGPPCPPCPAPEFLGGPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTISKAKGQPREPQVYTLPPCQDEL
TKNQVSLWCLVKGFYPSDIAVEWESNGQPENNYKTTPPVLDSDGSFFLYSRLTVDKSRWQEGNVFSCSVMHE
ALHNHYTQKSLSLSPG;;
CHA.7.518.4 scFv (VL-linker-VH) hIgG4 Variant 1
(SEQ ID NO: 3317)
DIQMTQSPSSLSASVGDRVTITCRVSENIYDVLAWYQQKPGKAPKLLIYEATNLAEGVPSRFSGSGSGTDFT
LTISSLQPEDFATYYCQHFWGTPYTFGQGTKLEIKggggsggggsggggsQVQLVQSGAEVKKPGASVKVSC
KASGYTFTDYNINWVRQAPGQGLEWMGYIYPYIGGSGYAQKFQGRVTMTRDTSTSTVYMELSSLRSEDTAVY
YCAREDKTARNAMDYWGQGTLVTVSSESKYGPPCPPCPAPPVA()GPSVFLFPPKPKDTLMISRTPEVTCVV
VDVKQEDPEVQFNWYVDGVEVHNAKTKPREEQFNSTYRVVSVLTVLHQDWLNGKEYKCKVSNKGLPSSIEKT
ISKAKGQPREPQVYTLPPSQEQMTKNQVKLTCLVKGFYPSDIAVEWESNGQPENNYKTTPPVLDSDGSFFLY
SRLTVDKSRWQEGNVFSCSVMHEALHNHYTQKSLSLSPG;
CHA.7.518.4 scFv (VH-linker-VL) hIgG4 Variant 1
(SEQ ID NO: 3327)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSggggsggggsggggsDIQMTQSPS
SLSASVGDRVTITCRVSENIYDVLAWYQQKPGKAPKLLIYEATNLAEGVPSRFSGSGSGTDFTLTISSLQPE
DFATYYCQHFWGTPYTFGQGTKLEIKESKYGPPCPPCPAPPVA()GPSVFLFPPKPKDTLMISRTPEVTCVV
VDVKQEDPEVQFNWYVDGVEVHNAKTKPREEQFNSTYRVVSVLTVLHQDWLNGKEYKCKVSNKGLPSSIEKT
ISKAKGQPREPQVYTLPPSQEQMTKNQVKLTCLVKGFYPSDIAVEWESNGQPENNYKTTPPVLDSDGSFFLY
SRLTVDKSRWQEGNVFSCSVMHEALHNHYTQKSLSLSPG;
CHA.7.518.4 scFv (VL-linker-VH) hIgG4 Variant 2
(SEQ ID NO: 3337)
DIQMTQSPSSLSASVGDRVTITCRVSENIYDVLAWYQQKPGKAPKLLIYEATNLAEGVPSRFSGSGSGTDFT
LTISSLQPEDFATYYCQHFWGTPYTFGQGTKLEIKggggsggggsggggsQVQLVQSGAEVKKPGASVKVSC
KASGYTFTDYNINWVRQAPGQGLEWMGYIYPYIGGSGYAQKFQGRVTMTRDTSTSTVYMELSSLRSEDTAVY
YCAREDKTARNAMDYWGQGTLVTVSSESKYGPPCPPCPAPPVA()GPSVFLFPPKPKDTLMISRTPEVTCVV
VDVKQEDPEVQFNWYVDGVEVHNAKTKPREEEFNSTYRVVSVLTVLHQDWLNGKEYKCKVSNKGLPSSIEKT
ISKAKGQPREPQVYTLPPSQEEMTKNQVSLTCDVSGFYPSDIAVEWESDGQPENNYKTTPPVLDSDGSFFLY
SRLTVDKSRWEEGDVFSCSVMHEALHNHYTQKSLSLSPG;
CHA.7.518.4 scFv (VH-linker-VL) hIgG4 Variant 2
(SEQ ID NO: 3347)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSggggsggggsggggsDIQMTQSPS
SLSASVGDRVTITCRVSENIYDVLAWYQQKPGKAPKLLIYEATNLAEGVPSRFSGSGSGTDFTLTISSLQPE
DFATYYCQHFWGTPYTFGQGTKLEIKESKYGPPCPPCPAPPVA()GPSVFLFPPKPKDTLMISRTPEVTCVV
VDVKQEDPEVQFNWYVDGVEVHNAKTKPREEEFNSTYRVVSVLTVLHQDWLNGKEYKCKVSNKGLPSSIEKT
ISKAKGQPREPQVYTLPPSQEEMTKNQVSLTCDVSGFYPSDIAVEWESDGQPENNYKTTPPVLDSDGSFFLY
SRLTVDKSRWEEGDVFSCSVMHEALHNHYTQKSLSLSPG;
CHA.7.518.4 hIgG4 Variant 1
(SEQ ID NO: 3357)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSASTKGPSVFPLAPCSRSTSESTAA
LGCLVKDYFPEPVTVSWNSGALTSGVHTFPAVLQSSGLYSLSSVVTVPSSSLGTKTYTCNVDHKPSNTKVDK
RVESKYGPPCPPCPAPPVA()GPSVFLFPPKPKDTLMISRTPEVTCVVVDVKQEDPEVQFNWYVDGVEVHNA
KTKPREEQFNSTYRVVSVLTVLHQDWLNGKEYKCKVSNKGLPSSIEKTISKAKGQPREPQVYTLPPSQEQMT
KNQVKLTCLVKGFYPSDIAVEWESNGQPENNYKTTPPVLDSDGSFFLYSRLTVDKSRWQEGNVFSCSVMHEA
LHNHYTQKSLSLSPG;
CHA.7.518.4 hIgG4 Variant 2
(SEQ ID NO: 3367)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSASTKGPSVFPLAPCSRSTSESTAA
LGCLVKDYFPEPVTVSWNSGALTSGVHTFPAVLQSSGLYSLSSVVTVPSSSLGTKTYTCNVDHKPSDTKVDK
RVESKYGPPCPPCPAPPVA()GPSVFLFPPKPKDTLMISRTPEVTCVVVDVKQEDPEVQFNWYVDGVEVHNA
KTKPREEEFNSTYRVVSVLTVLHQDWLNGKEYKCKVSNKGLPSSIEKTISKAKGQPREPQVYTLPPSQEEMT
KNQVSLTCDVSGFYPSDIAVEWESDGQPENNYKTTPPVLDSDGSFFLYSRLTVDKSRWEEGDVFSCSVMHEA
LHNHYTQKSLSLSPG;
CHA.7.518.4 VH Clambda-VL-CH1 Crossmab hIgG4 Fc Y349C E356D L358L T366S
L368A Y407V Hole
(SEQ ID NO: 3651)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSASVAAPSVFIFPPSDEQLKSGTAS
VVCLLNNFYPREAKVQWKVDNALQSGNSQESVTEQDSKDSTYSLSSTLTLSKADYEKHKVYACEVTHQGLSS
PVTKSFNRGECESKYGPPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQVCTL
PPSQDELTKNQVSLSCAVKGFYPSDIAVEWESNGQPENNYKTTPPVLDSDGSFFLVSRLTVDKSRWQEGNVF
SCSVMHEALHNHYTQKSLSLSPG;
CHA.7.518.4 VH Clambda-VL-CH1 Crossmab hIgG4 Fc Y354C E356D M348L T366W
Knob
(SEQ ID NO: 3652)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSASVAAPSVFIFPPSDEQLKSGTAS
VVCLLNNFYPREAKVQWKVDNALQSGNSQESVTEQDSKDSTYSLSSTLTLSKADYEKHKVYACEVTHQGLSS
PVTKSFNRGECESKYGPPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAKGQPREPQVYTL
PPCQDELTKNQVSLWCLVKGFYPSDIAVEWESNGQPENNYKTTPPVLDSDGSFFLYSRLTVDKSRWQEGNVF
SCSVMHEALHNHYTQKSLSLSPG;
CHA.7.518.4 VH hIgG4 Fc Y349C E356D L358L T366S L368A Y407V Hole
(SEQ ID NO: 3397)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSASASTKGPSVFPLAPCSRSTSEST
AALGCLVKDYFPEPVTVSWNSGALTSGVHTFPAVLQSSGLYSLSSVVTVPSSSLGTKTYTCNVDHKPSNTKV
DKRVESKYGPPCPPCPAPEFLGGPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTISKAKGQPREPQVCTLPPSQDEL
TKNQVSLSCAVKGFYPSDIAVEWESNGQPENNYKTTPPVLDSDGSFFLVSRLTVDKSRWQEGNVFSCSVMHE
ALHNHYTQKSLSLSPG;
or
CHA.7.518.4 VH hIgG4 Fc Y354C E356D M348L T366W Knob
(SEQ ID NO: 3407)
QVQLVQSGAEVKKPGASVKVSCKASGYTFTDYNINWVRQAPGQGLEWMGYIYPYIGGSGYAQKFQGRVTMTR
DTSTSTVYMELSSLRSEDTAVYYCAREDKTARNAMDYWGQGTLVTVSSASASTKGPSVFPLAPCSRSTSEST
AALGCLVKDYFPEPVTVSWNSGALTSGVHTFPAVLQSSGLYSLSSVVTVPSSSLGTKTYTCNVDHKPSNTKV
DKRVESKYGPPCPPCPAPEFLGGPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTISKAKGQPREPQVYTLPPCQDEL
TKNQVSLWCLVKGFYPSDIAVEWESNGQPENNYKTTPPVLDSDGSFFLYSRLTVDKSRWQEGNVFSCSVMHE
ALHNHYTQKSLSLSPG.

In some embodiments, the anti-TIGIT portion of the anti-PVRIG/anti-TIGIT bispecific antibody comprises one of the following anti-PVRIG sequences:

CPA.9.086 scFv (VL-linker-VH) hIgG4 Variant 1
(SEQ ID NO: 3417)
QSALTQPRSASGNPGQRVTISCSGSSSNMGRRPVNWYQQIPGTAPKLLIYSQNQRPSGVPDRFSGSQSGTSA
SLTISGLQSEDEAEYFCAVWDDIGRVLQLGGGTQLAVLggggsggggsggggsEVQLVETGGGLIQPGRSLR
LSCAASGFTFSSYAMHWVRQAPGKGLEWVAVISYAGEVKYYADSVKGRFTISRDNSKNTLYLQMNSLRAEDT
AVYYCARDPLPLHYYGMDVWGQGTTVTVSSESKYGPPCPPCPAPPVA()GPSVFLFPPKPKDTLMISRTPEV
TCVVVDVKQEDPEVQFNWYVDGVEVHNAKTKPREEQFNSTYRVVSVLTVLHQDWLNGKEYKCKVSNKGLPSS
IEKTISKAKGQPREPQVYTLPPSQEQMTKNQVKLTCLVKGFYPSDIAVEWESNGQPENNYKTTPPVLDSDGS
FFLYSRLTVDKSRWQEGNVFSCSVMHEALHNHYTQKSLSLSPG;
CPA.9.086 scFv (VH-linker-VL) hIgG4 Variant 1
(SEQ ID NO: 3427)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSggggsggggsggggsQSALTQPR
SASGNPGQRVTISCSGSSSNMGRRPVNWYQQIPGTAPKLLIYSQNQRPSGVPDRFSGSQSGTSASLTISGLQ
SEDEAEYFCAVWDDIGRVLQLGGGTQLAVLESKYGPPCPPCPAPPVA()GPSVFLFPPKPKDTLMISRTPEV
TCVVVDVKQEDPEVQFNWYVDGVEVHNAKTKPREEQFNSTYRVVSVLTVLHQDWLNGKEYKCKVSNKGLPSS
IEKTISKAKGQPREPQVYTLPPSQEQMTKNQVKLTCLVKGFYPSDIAVEWESNGQPENNYKTTPPVLDSDGS
FFLYSRLTVDKSRWQEGNVFSCSVMHEALHNHYTQKSLSLSPG;
CPA.9.086 scFv (VL-linker-VH) hIgG4 Variant 2
(SEQ ID NO: 3437)
QSALTQPRSASGNPGQRVTISCSGSSSNMGRRPVNWYQQIPGTAPKLLIYSQNQRPSGVPDRFSGSQSGTSA
SLTISGLQSEDEAEYFCAVWDDIGRVLQLGGGTQLAVLggggsggggsggggsEVQLVETGGGLIQPGRSLR
LSCAASGFTFSSYAMHWVRQAPGKGLEWVAVISYAGEVKYYADSVKGRFTISRDNSKNTLYLQMNSLRAEDT
AVYYCARDPLPLHYYGMDVWGQGTTVTVSSESKYGPPCPPCPAPPVA()GPSVFLFPPKPKDTLMISRTPEV
TCVVVDVKQEDPEVQFNWYVDGVEVHNAKTKPREEEFNSTYRVVSVLTVLHQDWLNGKEYKCKVSNKGLPSS
IEKTISKAKGQPREPQVYTLPPSQEEMTKNQVSLTCDVSGFYPSDIAVEWESDGQPENNYKTTPPVLDSDGS
FFLYSRLTVDKSRWEEGDVFSCSVMHEALHNHYTQKSLSLSPG;
CPA.9.086 scFv (VH-linker-VL) hIgG4 Variant 2
(SEQ ID NO: 3447)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSggggsggggsggggsQSALTQPR
SASGNPGQRVTISCSGSSSNMGRRPVNWYQQIPGTAPKLLIYSQNQRPSGVPDRFSGSQSGTSASLTISGLQ
SEDEAEYFCAVWDDIGRVLQLGGGTQLAVLESKYGPPCPPCPAPPVA()GPSVFLFPPKPKDTLMISRTPEV
TCVVVDVKQEDPEVQFNWYVDGVEVHNAKTKPREEEFNSTYRVVSVLTVLHQDWLNGKEYKCKVSNKGLPSS
IEKTISKAKGQPREPQVYTLPPSQEEMTKNQVSLTCDVSGFYPSDIAVEWESDGQPENNYKTTPPVLDSDGS
FFLYSRLTVDKSRWEEGDVFSCSVMHEALHNHYTQKSLSLSPG;
CPA.9.086 hIgG4 Variant 1
(SEQ ID NO: 3457)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASTKGPSVFPLAPCSRSTSESTA
ALGCLVKDYFPEPVTVSWNSGALTSGVHTFPAVLQSSGLYSLSSVVTVPSSSLGTKTYTCNVDHKPSDTKVD
KRVESKYGPPCPPCPAPPVA()GPSVFLFPPKPKDTLMISRTPEVTCVVVDVKQEDPEVQFNWYVDGVEVHN
AKTKPREEQFNSTYRVVSVLTVLHQDWLNGKEYKCKVSNKGLPSSIEKTISKAKGQPREPQVYTLPPSQEQM
TKNQVKLTCLVKGFYPSDIAVEWESNGQPENNYKTTPPVLDSDGSFFLYSRLTVDKSRWQEGNVFSCSVMHE
ALHNHYTQKSLSLSPG;
CPA.9.086 hIgG4 Variant 2
(SEQ ID NO: 3467)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASTKGPSVFPLAPCSRSTSESTA
ALGCLVKDYFPEPVTVSWNSGALTSGVHTFPAVLQSSGLYSLSSVVTVPSSSLGTKTYTCNVDHKPSDTKVD
KRVESKYGPPCPPCPAPPVA()GPSVFLFPPKPKDTLMISRTPEVTCVVVDVKQEDPEVQFNWYVDGVEVHN
AKTKPREEEFNSTYRVVSVLTVLHQDWLNGKEYKCKVSNKGLPSSIEKTISKAKGQPREPQVYTLPPSQEEM
TKNQVSLTCDVSGFYPSDIAVEWESDGQPENNYKTTPPVLDSDGSFFLYSRLTVDKSRWEEGDVFSCSVMHE
ALHNHYTQKSLSLSPG;
CPA.9.086 VH Clambda-VL-CH1 Crossmab hIgG4 Fc Y349C E356D L358L T366S
L368A Y407V Hole
(SEQ ID NO: 3477)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASGQPKAAPSVTLFPPSSEELQA
NKATLVCLISDFYPGAVTVAWKADSSPVKAGVETTTPSKQSNNKYAASSYLSLTPEQWKSHRSYSCQVTHEG
STVEKTVAPTECSESKYGPPCPPCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREPQVC
TLPPSQDELTKNQVSLSCAVKGFYPSDIAVEWESNGQPENNYKTTPPVLDSDGSFFLVSRLTVDKSRWQEGN
VFSCSVMHEALHNHYTQKSLSLSPG;
CPA.9.086 VH Clambda-VL-CH1 Crossmab hIgG4 Fc Y354C E356D M348L T366W
Knob
(SEQ ID NO: 3487)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASGQPKAAPSVTLFPPSSEELQA
NKATLVCLISDFYPGAVTVAWKADSSPVKAGVETTTPSKQSNNKYAASSYLSLTPEQWKSHRSYSCQVTHEG
STVEKTVAPTECSESKYGPPCPPCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREPQVY
TLPPCQDELTKNQVSLWCLVKGFYPSDIAVEWESNGQPENNYKTTPPVLDSDGSFFLYSRLTVDKSRWQEGN
VFSCSVMHEALHNHYTQKSLSLSPG;
CPA.9.086 VH hIgG4 Fc Y349C E356D L358L T366S L368A Y407V Hole
(SEQ ID NO: 3497)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASASTKGPSVFPLAPCSRSTSES
TAALGCLVKDYFPEPVTVSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAKGQPREPQVCTLPPSQDE
LTKNQVSLSCAVKGFYPSDIAVEWESNGQPENNYKTTPPVLDSDGSFFLVSRLTVDKSRWQEGNVFSCSVMH
EALHNHYTQKSLSLSPG;
CPA.9.086 VH hIgG4 Fc Y354C E356D M348L T366W Knob
(SEQ ID NO: 3507)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASASTKGPSVFPLAPCSRSTSES
TAALGCLVKDYFPEPVTVSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAKGQPREPQVYTLPPCQDE
LTKNQVSLWCLVKGFYPSDIAVEWESNGQPENNYKTTPPVLDSDGSFFLYSRLTVDKSRWQEGNVFSCSVMH
EALHNHYTQKSLSLSPG;
CHA.9.547.18 scFv (VL-linker-VH) hIgG4 Variant 1
(SEQ ID NO: 3517)
DIQMTQSPSSLSASVGDRITITCRASQSMAIWLSWYQQKPGKAPKLLIYKASKSHTGVPSRFSGSGSGTDFT
LTISSLQPEDFATYYCQQGQSYPYTFGQGTKLEIKggggsggggsggggsEVQLVESGGGLVQPGGSLRLSC
AASGFTFSSYIMSWVRQAPGKGLEWVATISGGSKGQYYADSVKGRFTISRDNSKNTLYLQMNSLRAEDTAVY
YCAKWLLSYYAMDYWGQGTLVTVSSESKYGPPCPPCPAPPVA()GPSVFLFPPKPKDTLMISRTPEVTCVVV
DVKQEDPEVQFNWYVDGVEVHNAKTKPREEQFNSTYRVVSVLTVLHQDWLNGKEYKCKVSNKGLPSSIEKTI
SKAKGQPREPQVYTLPPSQEQMTKNQVKLTCLVKGFYPSDIAVEWESNGQPENNYKTTPPVLDSDGSFFLYS
RLTVDKSRWQEGNVFSCSVMHEALHNHYTQKSLSLSPG;
CHA.9.547.18 scFv (VH-linker-VL) hIgG4 Variant 1
(SEQ ID NO: 3527)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSggggsggggsggggsDIQMTQSPSS
LSASVGDRITITCRASQSMAIWLSWYQQKPGKAPKLLIYKASKSHTGVPSRFSGSGSGTDFTLTISSLQPED
FATYYCQQGQSYPYTFGQGTKLEIKESKYGPPCPPCPAPPVA()GPSVFLFPPKPKDTLMISRTPEVTCVVV
DVKQEDPEVQFNWYVDGVEVHNAKTKPREEQFNSTYRVVSVLTVLHQDWLNGKEYKCKVSNKGLPSSIEKTI
SKAKGQPREPQVYTLPPSQEQMTKNQVKLTCLVKGFYPSDIAVEWESNGQPENNYKTTPPVLDSDGSFFLYS
RLTVDKSRWQEGNVFSCSVMHEALHNHYTQKSLSLSPG;
CHA.9.547.18 scFv (VL-linker-VH) hIgG4 Variant 2
(SEQ ID NO: 3537)
DIQMTQSPSSLSASVGDRITITCRASQSMAIWLSWYQQKPGKAPKLLIYKASKSHTGVPSRFSGSGSGTDFT
LTISSLQPEDFATYYCQQGQSYPYTFGQGTKLEIKggggsggggsggggsEVQLVESGGGLVQPGGSLRLSC
AASGFTFSSYIMSWVRQAPGKGLEWVATISGGSKGQYYADSVKGRFTISRDNSKNTLYLQMNSLRAEDTAVY
YCAKWLLSYYAMDYWGQGTLVTVSSESKYGPPCPPCPAPPVA()GPSVFLFPPKPKDTLMISRTPEVTCVVV
DVKQEDPEVQFNWYVDGVEVHNAKTKPREEEFNSTYRVVSVLTVLHQDWLNGKEYKCKVSNKGLPSSIEKTI
SKAKGQPREPQVYTLPPSQEEMTKNQVSLTCDVSGFYPSDIAVEWESDGQPENNYKTTPPVLDSDGSFFLYS
RLTVDKSRWEEGDVFSCSVMHEALHNHYTQKSLSLSPG;
CHA.9.547.18 scFv (VH-linker-VL) hIgG4 Variant 2
(SEQ ID NO: 3547)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSggggsggggsggggsDIQMTQSPSS
LSASVGDRITITCRASQSMAIWLSWYQQKPGKAPKLLIYKASKSHTGVPSRFSGSGSGTDFTLTISSLQPED
FATYYCQQGQSYPYTFGQGTKLEIKESKYGPPCPPCPAPPVA()GPSVFLFPPKPKDTLMISRTPEVTCVVV
DVKQEDPEVQFNWYVDGVEVHNAKTKPREEEFNSTYRVVSVLTVLHQDWLNGKEYKCKVSNKGLPSSIEKTI
SKAKGQPREPQVYTLPPSQEEMTKNQVSLTCDVSGFYPSDIAVEWESDGQPENNYKTTPPVLDSDGSFFLYS
RLTVDKSRWEEGDVFSCSVMHEALHNHYTQKSLSLSPG;
CHA.9.547.18 hIgG4 Variant 1
(SEQ ID NO: 3557)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSNTKVDKR
VESKYGPPCPPCPAPPVA()GPSVFLFPPKPKDTLMISRTPEVTCVVVDVKQEDPEVQFNWYVDGVEVHNAK
TKPREEQFNSTYRVVSVLTVLHQDWLNGKEYKCKVSNKGLPSSIEKTISKAKGQPREPQVYTLPPSQEQMTK
NQVKLTCLVKGFYPSDIAVEWESNGQPENNYKTTPPVLDSDGSFFLYSRLTVDKSRWQEGNVFSCSVMHEAL
HNHYTQKSLSLSPG;
CHA.9.547.18 hIgG4 Variant 2
(SEQ ID NO: 3567)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSDTKVDKR
VESKYGPPCPPCPAPPVA()GPSVFLFPPKPKDTLMISRTPEVTCVVVDVKQEDPEVQFNWYVDGVEVHNAK
TKPREEEFNSTYRVVSVLTVLHQDWLNGKEYKCKVSNKGLPSSIEKTISKAKGQPREPQVYTLPPSQEEMTK
NQVSLTCDVSGFYPSDIAVEWESDGQPENNYKTTPPVLDSDGSFFLYSRLTVDKSRWEEGDVFSCSVMHEAL
HNHYTQKSLSLSPG;
CHA.9.547.18 VH Clambda-VL-CH1 Crossmab hIgG4 Fc Y349C E356D L358L
T366S L368A Y407V Hole
(SEQ ID NO: 3653)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASVAAPSVFIFPPSDEQLKSGTASV
VCLLNNFYPREAKVQWKVDNALQSGNSQESVTEQDSKDSTYSLSSTLTLSKADYEKHKVYACEVTHQGLSSP
VTKSFNRGECESKYGPPCPPCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISKAKGQPREPQVCTLP
PSQDELTKNQVSLSCAVKGFYPSDIAVEWESNGQPENNYKTTPPVLDSDGSFFLVSRLTVDKSRWQEGNVFS
CSVMHEALHNHYTQKSLSLSPG;
CHA.9.547.18 VH Clambda-VL-CH1 Crossmab hIgG4 Fc Y354C E356D M348L
T366W Knob
(SEQ ID NO: 3654)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASVAAPSVFIFPPSDEQLKSGTASV
VCLLNNFYPREAKVQWKVDNALQSGNSQESVTEQDSKDSTYSLSSTLTLSKADYEKHKVYACEVTHQGLSSP
VTKSFNRGECESKYGPPCPPCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISKAKGQPREPQVYTLP
PCQDELTKNQVSLWCLVKGFYPSDIAVEWESNGQPENNYKTTPPVLDSDGSFFLYSRLTVDKSRWQEGNVFS
CSVMHEALHNHYTQKSLSLSPG;
CHA.9.547.18 VH hIgG4 Fc Y349C E356D L358L T366S L368A Y407V Hole
(SEQ ID NO: 3597)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASASTKGPSVFPLAPCSRSTSESTA
ALGCLVKDYFPEPVTVSWNSGALTSGVHTFPAVLQSSGLYSLSSVVTVPSSSLGTKTYTCNVDHKPSNTKVD
KRVESKYGPPCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVCTLPPSQDELT
KNQVSLSCAVKGFYPSDIAVEWESNGQPENNYKTTPPVLDSDGSFFLVSRLTVDKSRWQEGNVFSCSVMHEA
LHNHYTQKSLSLSPG;
or
CHA.9.547.18 VH hIgG4 Fc Y354C E356D M348L T366W Knob
(SEQ ID NO: 3607)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASASTKGPSVFPLAPCSRSTSESTA
ALGCLVKDYFPEPVTVSWNSGALTSGVHTFPAVLQSSGLYSLSSVVTVPSSSLGTKTYTCNVDHKPSNTKVD
KRVESKYGPPCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVYTLPPCQDELT
KNQVSLWCLVKGFYPSDIAVEWESNGQPENNYKTTPPVLDSDGSFFLYSRLTVDKSRWQEGNVFSCSVMHEA
LHNHYTQKSLSLSPG.

In some embodiments, the anti-PVRIG portion of the anti-PVRIG/anti-TIGIT bispecific antibody comprises the following anti-PVRIG sequence

CHA.7.518.1 scFv (VL-linker-VH) hIgG4 Variant 1

(SEQ ID NO: 3217)

DIQMTQSPSSLSASVGDRVTITCRVSENIYSNLAWYQQKPGKAPKLLIYE
ATNLAEGVPSRFSGSGSGTDFTLTISSLQPEDFATYYCQHFWGTPYTFGQ
GTKLEIKggggsggggsggggsQVQLVQSGAEVKKPGASVKVSCKASGYT
FTDYNINWVRQAPGQGLEWMGYIYPYIGGSGYAQKFQGRVTMTRDTSTST
VYMELSSLRSEDTAVYYCAREDKTARNAMDYWGQGTLVTVSSESKYGPPC
PPCPAPPVA()GPSVFLFPPKPKDTLMISRTPEVTCVVVDVKQEDPEVQF
NWYVDGVEVHNAKTKPREEQFNSTYRVVSVLTVLHQDWLNGKEYKCKVSN
KGLPSSIEKTISKAKGQPREPQVYTLPPSQEQMTKNQVKLTCLVKGFYPS
DIAVEWESNGQPENNYKTTPPVLDSDGSFFLYSRLTVDKSRWQEGNVFSC
SVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 scFv (VL-linker-VH) hIgG4 Variant 2
(SEQ ID NO: 3437)
QSALTQPRSASGNPGQRVTISCSGSSSNMGRRPVNWYQQIPGTAPKLLIYSQNQRPSGVPDRFSGSQSGTSA
SLTISGLQSEDEAEYFCAVWDDIGRVLQLGGGTQLAVLggggsggggsggggsEVQLVETGGGLIQPGRSLR
LSCAASGFTFSSYAMHWVRQAPGKGLEWVAVISYAGEVKYYADSVKGRFTISRDNSKNTLYLQMNSLRAEDT
AVYYCARDPLPLHYYGMDVWGQGTTVTVSSESKYGPPCPPCPAPPVA()GPSVFLFPPKPKDTLMISRTPEV
TCVVVDVKQEDPEVQFNWYVDGVEVHNAKTKPREEEFNSTYRVVSVLTVLHQDWLNGKEYKCKVSNKGLPSS
IEKTISKAKGQPREPQVYTLPPSQEEMTKNQVSLTCDVSGFYPSDIAVEWESDGQPENNYKTTPPVLDSDGS
FFLYSRLTVDKSRWEEGDVFSCSVMHEALHNHYTQKSLSLSPG;
CPA.9.086 scFv (VH-linker-VL) hIgG4 Variant 2
(SEQ ID NO: 3447)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSggggsggggsggggsQSALTQPR
SASGNPGQRVTISCSGSSSNMGRRPVNWYQQIPGTAPKLLIYSQNQRPSGVPDRFSGSQSGTSASLTISGLQ
SEDEAEYFCAVWDDIGRVLQLGGGTQLAVLESKYGPPCPPCPAPPVA()GPSVFLFPPKPKDTLMISRTPEV
TCVVVDVKQEDPEVQFNWYVDGVEVHNAKTKPREEEFNSTYRVVSVLTVLHQDWLNGKEYKCKVSNKGLPSS
IEKTISKAKGQPREPQVYTLPPSQEEMTKNQVSLTCDVSGFYPSDIAVEWESDGQPENNYKTTPPVLDSDGS
FFLYSRLTVDKSRWEEGDVFSCSVMHEALHNHYTQKSLSLSPG;
CPA.9.086 hIgG4 Variant 2
(SEQ ID NO: 3467)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASTKGPSVFPLAPCSRSTSESTA
ALGCLVKDYFPEPVTVSWNSGALTSGVHTFPAVLQSSGLYSLSSVVTVPSSSLGTKTYTCNVDHKPSDTKVD
KRVESKYGPPCPPCPAPPVA()GPSVFLFPPKPKDTLMISRTPEVTCVVVDVKQEDPEVQFNWYVDGVEVHN
AKTKPREEEFNSTYRVVSVLTVLHQDWLNGKEYKCKVSNKGLPSSIEKTISKAKGQPREPQVYTLPPSQEEM
TKNQVSLTCDVSGFYPSDIAVEWESDGQPENNYKTTPPVLDSDGSFFLYSRLTVDKSRWEEGDVFSCSVMHE
ALHNHYTQKSLSLSPG;
CHA.9.547.18 scFv (VL-linker-VH) hIgG4 Variant 2
(SEQ ID NO: 3537)
DIQMTQSPSSLSASVGDRITITCRASQSMAIWLSWYQQKPGKAPKLLIYKASKSHTGVPSRFSGSGSGTDFT
LTISSLQPEDFATYYCQQGQSYPYTFGQGTKLEIKggggsggggsggggsEVQLVESGGGLVQPGGSLRLSC
AASGFTFSSYIMSWVRQAPGKGLEWVATISGGSKGQYYADSVKGRFTISRDNSKNTLYLQMNSLRAEDTAVY
YCAKWLLSYYAMDYWGQGTLVTVSSESKYGPPCPPCPAPPVA()GPSVFLFPPKPKDTLMISRTPEVTCVVV
DVKQEDPEVQFNWYVDGVEVHNAKTKPREEEFNSTYRVVSVLTVLHQDWLNGKEYKCKVSNKGLPSSIEKTI
SKAKGQPREPQVYTLPPSQEEMTKNQVSLTCDVSGFYPSDIAVEWESDGQPENNYKTTPPVLDSDGSFFLYS
RLTVDKSRWEEGDVFSCSVMHEALHNHYTQKSLSLSPG;
CHA.9.547.18 scFv (VH-linker-VL) hIgG4 Variant 2
(SEQ ID NO: 3547)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSggggsggggsggggsDIQMTQSPSS
LSASVGDRITITCRASQSMAIWLSWYQQKPGKAPKLLIYKASKSHTGVPSRFSGSGSGTDFTLTISSLQPED
FATYYCQQGQSYPYTFGQGTKLEIKESKYGPPCPPCPAPPVA()GPSVFLFPPKPKDTLMISRTPEVTCVVV
DVKQEDPEVQFNWYVDGVEVHNAKTKPREEEFNSTYRVVSVLTVLHQDWLNGKEYKCKVSNKGLPSSIEKTI
SKAKGQPREPQVYTLPPSQEEMTKNQVSLTCDVSGFYPSDIAVEWESDGQPENNYKTTPPVLDSDGSFFLYS
RLTVDKSRWEEGDVFSCSVMHEALHNHYTQKSLSLSPG;
and
CHA.9.547.18 hIgG4 Variant 2
(SEQ ID NO: 3567)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSDTKVDKR
VESKYGPPCPPCPAPPVA()GPSVFLFPPKPKDTLMISRTPEVTCVVVDVKQEDPEVQFNWYVDGVEVHNAK
TKPREEEFNSTYRVVSVLTVLHQDWLNGKEYKCKVSNKGLPSSIEKTISKAKGQPREPQVYTLPPSQEEMTK
NQVSLTCDVSGFYPSDIAVEWESDGQPENNYKTTPPVLDSDGSFFLYSRLTVDKSRWEEGDVFSCSVMHEAL
HNHYTQKSLSLSPG.

In some embodiments, the anti-PVRIG portion of the anti-PVRIG/anti-TIGIT bispecific antibody comprises the following anti-PVRIG sequence

CHA.7.518.1 scFv (VL-linker-VH) hIgG4 Variant 1

(SEQ ID NO: 3217)

DIQMTQSPSSLSASVGDRVTITCRVSENIYSNLAWYQQKPGKAPKLLIYE
ATNLAEGVPSRFSGSGSGTDFTLTISSLQPEDFATYYCQHFWGTPYTFGQ
GTKLEIKggggsggggsggggsQVQLVQSGAEVKKPGASVKVSCKASGYT
FTDYNINWVRQAPGQGLEWMGYIYPYIGGSGYAQKFQGRVTMTRDTSTST
VYMELSSLRSEDTAVYYCAREDKTARNAMDYWGQGTLVTVSSESKYGPPC
PPCPAPPVA()GPSVFLFPPKPKDTLMISRTPEVTCVVVDVKQEDPEVQF
NWYVDGVEVHNAKTKPREEQFNSTYRVVSVLTVLHQDWLNGKEYKCKVSN
KGLPSSIEKTISKAKGQPREPQVYTLPPSQEQMTKNQVKLTCLVKGFYPS
DIAVEWESNGQPENNYKTTPPVLDSDGSFFLYSRLTVDKSRWQEGNVFSC
SVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 scFv (VL-linker-VH) hIgG4 Variant 2
(SEQ ID NO: 3437)
QSALTQPRSASGNPGQRVTISCSGSSSNMGRRPVNWYQQIPGTAPKLLIYSQNQRPSGVPDRFSGSQSGTSA
SLTISGLQSEDEAEYFCAVWDDIGRVLQLGGGTQLAVLggggsggggsggggsEVQLVETGGGLIQPGRSLR
LSCAASGFTFSSYAMHWVRQAPGKGLEWVAVISYAGEVKYYADSVKGRFTISRDNSKNTLYLQMNSLRAEDT
AVYYCARDPLPLHYYGMDVWGQGTTVTVSSESKYGPPCPPCPAPPVA()GPSVFLFPPKPKDTLMISRTPEV
TCVVVDVKQEDPEVQFNWYVDGVEVHNAKTKPREEEFNSTYRVVSVLTVLHQDWLNGKEYKCKVSNKGLPSS
IEKTISKAKGQPREPQVYTLPPSQEEMTKNQVSLTCDVSGFYPSDIAVEWESDGQPENNYKTTPPVLDSDGS
FFLYSRLTVDKSRWEEGDVFSCSVMHEALHNHYTQKSLSLSPG;
CPA.9.086 scFv (VH-linker-VL) hIgG4 Variant 2
(SEQ ID NO: 3447)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSggggsggggsggggsQSALTQPR
SASGNPGQRVTISCSGSSSNMGRRPVNWYQQIPGTAPKLLIYSQNQRPSGVPDRFSGSQSGTSASLTISGLQ
SEDEAEYFCAVWDDIGRVLQLGGGTQLAVLESKYGPPCPPCPAPPVA()GPSVFLFPPKPKDTLMISRTPEV
TCVVVDVKQEDPEVQFNWYVDGVEVHNAKTKPREEEFNSTYRVVSVLTVLHQDWLNGKEYKCKVSNKGLPSS
IEKTISKAKGQPREPQVYTLPPSQEEMTKNQVSLTCDVSGFYPSDIAVEWESDGQPENNYKTTPPVLDSDGS
FFLYSRLTVDKSRWEEGDVFSCSVMHEALHNHYTQKSLSLSPG;
CPA.9.086 hIgG4 Variant 2
(SEQ ID NO: 3467)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASTKGPSVFPLAPCSRSTSESTA
ALGCLVKDYFPEPVTVSWNSGALTSGVHTFPAVLQSSGLYSLSSVVTVPSSSLGTKTYTCNVDHKPSDTKVD
KRVESKYGPPCPPCPAPPVA()GPSVFLFPPKPKDTLMISRTPEVTCVVVDVKQEDPEVQFNWYVDGVEVHN
AKTKPREEEFNSTYRVVSVLTVLHQDWLNGKEYKCKVSNKGLPSSIEKTISKAKGQPREPQVYTLPPSQEEM
TKNQVSLTCDVSGFYPSDIAVEWESDGQPENNYKTTPPVLDSDGSFFLYSRLTVDKSRWEEGDVFSCSVMHE
ALHNHYTQKSLSLSPG;
CHA.9.547.18 scFv (VL-linker-VH) hIgG4 Variant 2
(SEQ ID NO: 3537)
DIQMTQSPSSLSASVGDRITITCRASQSMAIWLSWYQQKPGKAPKLLIYKASKSHTGVPSRFSGSGSGTDFT
LTISSLQPEDFATYYCQQGQSYPYTFGQGTKLEIKggggsggggsggggsEVQLVESGGGLVQPGGSLRLSC
AASGFTFSSYIMSWVRQAPGKGLEWVATISGGSKGQYYADSVKGRFTISRDNSKNTLYLQMNSLRAEDTAVY
YCAKWLLSYYAMDYWGQGTLVTVSSESKYGPPCPPCPAPPVA()GPSVFLFPPKPKDTLMISRTPEVTCVVV
DVKQEDPEVQFNWYVDGVEVHNAKTKPREEEFNSTYRVVSVLTVLHQDWLNGKEYKCKVSNKGLPSSIEKTI
SKAKGQPREPQVYTLPPSQEEMTKNQVSLTCDVSGFYPSDIAVEWESDGQPENNYKTTPPVLDSDGSFFLYS
RLTVDKSRWEEGDVFSCSVMHEALHNHYTQKSLSLSPG;
CHA.9.547.18 scFv (VH-linker-VL) hIgG4 Variant 2
(SEQ ID NO: 3547)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSggggsggggsggggsDIQMTQSPSS
LSASVGDRITITCRASQSMAIWLSWYQQKPGKAPKLLIYKASKSHTGVPSRFSGSGSGTDFTLTISSLQPED
FATYYCQQGQSYPYTFGQGTKLEIKESKYGPPCPPCPAPPVA()GPSVFLFPPKPKDTLMISRTPEVTCVVV
DVKQEDPEVQFNWYVDGVEVHNAKTKPREEEFNSTYRVVSVLTVLHQDWLNGKEYKCKVSNKGLPSSIEKTI
SKAKGQPREPQVYTLPPSQEEMTKNQVSLTCDVSGFYPSDIAVEWESDGQPENNYKTTPPVLDSDGSFFLYS
RLTVDKSRWEEGDVFSCSVMHEALHNHYTQKSLSLSPG;
and
CHA.9.547.18 hIgG4 Variant 2
(SEQ ID NO: 3567)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSDTKVDKR
VESKYGPPCPPCPAPPVA()GPSVFLFPPKPKDTLMISRTPEVTCVVVDVKQEDPEVQFNWYVDGVEVHNAK
TKPREEEFNSTYRVVSVLTVLHQDWLNGKEYKCKVSNKGLPSSIEKTISKAKGQPREPQVYTLPPSQEEMTK
NQVSLTCDVSGFYPSDIAVEWESDGQPENNYKTTPPVLDSDGSFFLYSRLTVDKSRWEEGDVFSCSVMHEAL
HNHYTQKSLSLSPG.

In some embodiments, the anti-PVRIG portion of the anti-PVRIG/anti-TIGIT bispecific antibody comprises the following anti-PVRIG sequence

CHA.7.518.1 scFv (VH-linker-VL) hIgG4 Variant 1

(SEQ ID NO: 3227)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSggggsggggsggggsDIQMTQSPSSLSASV
GDRVTITCRVSENIYSNLAWYQQKPGKAPKLLIYEATNLAEGVPSRFSGS
GSGTDFTLTISSLQPEDFATYYCQHFWGTPYTFGQGTKLEIKESKYGPPC
PPCPAPPVA()GPSVFLFPPKPKDTLMISRTPEVTCVVVDVKQEDPEVQF
NWYVDGVEVHNAKTKPREEQFNSTYRVVSVLTVLHQDWLNGKEYKCKVSN
KGLPSSIEKTISKAKGQPREPQVYTLPPSQEQMTKNQVKLTCLVKGFYPS
DIAVEWESNGQPENNYKTTPPVLDSDGSFFLYSRLTVDKSRWQEGNVFSC
SVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 scFv (VL-linker-VH) hIgG4 Variant 2
(SEQ ID NO: 3437)
QSALTQPRSASGNPGQRVTISCSGSSSNMGRRPVNWYQQIPGTAPKLLIYSQNQRPSGVPDRFSGSQSGTSA
SLTISGLQSEDEAEYFCAVWDDIGRVLQLGGGTQLAVLggggsggggsggggsEVQLVETGGGLIQPGRSLR
LSCAASGFTFSSYAMHWVRQAPGKGLEWVAVISYAGEVKYYADSVKGRFTISRDNSKNTLYLQMNSLRAEDT
AVYYCARDPLPLHYYGMDVWGQGTTVTVSSESKYGPPCPPCPAPPVA()GPSVFLFPPKPKDTLMISRTPEV
TCVVVDVKQEDPEVQFNWYVDGVEVHNAKTKPREEEFNSTYRVVSVLTVLHQDWLNGKEYKCKVSNKGLPSS
IEKTISKAKGQPREPQVYTLPPSQEEMTKNQVSLTCDVSGFYPSDIAVEWESDGQPENNYKTTPPVLDSDGS
FFLYSRLTVDKSRWEEGDVFSCSVMHEALHNHYTQKSLSLSPG;
CPA.9.086 scFv (VH-linker-VL) hIgG4 Variant 2
(SEQ ID NO: 3447)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSggggsggggsggggsQSALTQPR
SASGNPGQRVTISCSGSSSNMGRRPVNWYQQIPGTAPKLLIYSQNQRPSGVPDRFSGSQSGTSASLTISGLQ
SEDEAEYFCAVWDDIGRVLQLGGGTQLAVLESKYGPPCPPCPAPPVA()GPSVFLFPPKPKDTLMISRTPEV
TCVVVDVKQEDPEVQFNWYVDGVEVHNAKTKPREEEFNSTYRVVSVLTVLHQDWLNGKEYKCKVSNKGLPSS
IEKTISKAKGQPREPQVYTLPPSQEEMTKNQVSLTCDVSGFYPSDIAVEWESDGQPENNYKTTPPVLDSDGS
FFLYSRLTVDKSRWEEGDVFSCSVMHEALHNHYTQKSLSLSPG;
CPA.9.086 hIgG4 Variant 2
(SEQ ID NO: 3467)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASTKGPSVFPLAPCSRSTSESTA
ALGCLVKDYFPEPVTVSWNSGALTSGVHTFPAVLQSSGLYSLSSVVTVPSSSLGTKTYTCNVDHKPSDTKVD
KRVESKYGPPCPPCPAPPVA()GPSVFLFPPKPKDTLMISRTPEVTCVVVDVKQEDPEVQFNWYVDGVEVHN
AKTKPREEEFNSTYRVVSVLTVLHQDWLNGKEYKCKVSNKGLPSSIEKTISKAKGQPREPQVYTLPPSQEEM
TKNQVSLTCDVSGFYPSDIAVEWESDGQPENNYKTTPPVLDSDGSFFLYSRLTVDKSRWEEGDVFSCSVMHE
ALHNHYTQKSLSLSPG;
CHA.9.547.18 scFv (VL-linker-VH) hIgG4 Variant 2
(SEQ ID NO: 3537)
DIQMTQSPSSLSASVGDRITITCRASQSMAIWLSWYQQKPGKAPKLLIYKASKSHTGVPSRFSGSGSGTDFT
LTISSLQPEDFATYYCQQGQSYPYTFGQGTKLEIKggggsggggsggggsEVQLVESGGGLVQPGGSLRLSC
AASGFTFSSYIMSWVRQAPGKGLEWVATISGGSKGQYYADSVKGRFTISRDNSKNTLYLQMNSLRAEDTAVY
YCAKWLLSYYAMDYWGQGTLVTVSSESKYGPPCPPCPAPPVA()GPSVFLFPPKPKDTLMISRTPEVTCVVV
DVKQEDPEVQFNWYVDGVEVHNAKTKPREEEFNSTYRVVSVLTVLHQDWLNGKEYKCKVSNKGLPSSIEKTI
SKAKGQPREPQVYTLPPSQEEMTKNQVSLTCDVSGFYPSDIAVEWESDGQPENNYKTTPPVLDSDGSFFLYS
RLTVDKSRWEEGDVFSCSVMHEALHNHYTQKSLSLSPG;
CHA.9.547.18 scFv (VH-linker-VL) hIgG4 Variant 2
(SEQ ID NO: 3547)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSggggsggggsggggsDIQMTQSPSS
LSASVGDRITITCRASQSMAIWLSWYQQKPGKAPKLLIYKASKSHTGVPSRFSGSGSGTDFTLTISSLQPED
FATYYCQQGQSYPYTFGQGTKLEIKESKYGPPCPPCPAPPVA()GPSVFLFPPKPKDTLMISRTPEVTCVVV
DVKQEDPEVQFNWYVDGVEVHNAKTKPREEEFNSTYRVVSVLTVLHQDWLNGKEYKCKVSNKGLPSSIEKTI
SKAKGQPREPQVYTLPPSQEEMTKNQVSLTCDVSGFYPSDIAVEWESDGQPENNYKTTPPVLDSDGSFFLYS
RLTVDKSRWEEGDVFSCSVMHEALHNHYTQKSLSLSPG;
and
CHA.9.547.18 hIgG4 Variant 2
(SEQ ID NO: 3567)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSDTKVDKR
VESKYGPPCPPCPAPPVA()GPSVFLFPPKPKDTLMISRTPEVTCVVVDVKQEDPEVQFNWYVDGVEVHNAK
TKPREEEFNSTYRVVSVLTVLHQDWLNGKEYKCKVSNKGLPSSIEKTISKAKGQPREPQVYTLPPSQEEMTK
NQVSLTCDVSGFYPSDIAVEWESDGQPENNYKTTPPVLDSDGSFFLYSRLTVDKSRWEEGDVFSCSVMHEAL
HNHYTQKSLSLSPG.

In some embodiments, the anti-PVRIG portion of the anti-PVRIG/anti-TIGIT bispecific antibody comprises the following anti-PVRIG sequence

CHA.7.518.1 scFv (VL-linker-VH) hIgG4 Variant 2

(SEQ ID NO: 3237)

DIQMTQSPSSLSASVGDRVTITCRVSENIYSNLAWYQQKPGKAPKLLIYE
ATNLAEGVPSRFSGSGSGTDFTLTISSLQPEDFATYYCQHFWGTPYTFGQ
GTKLEIKggggsggggsggggsQVQLVQSGAEVKKPGASVKVSCKASGYT
FTDYNINWVRQAPGQGLEWMGYIYPYIGGSGYAQKFQGRVTMTRDTSTST
VYMELSSLRSEDTAVYYCAREDKTARNAMDYWGQGTLVTVSSESKYGPPC
PPCPAPPVA()GPSVFLFPPKPKDTLMISRTPEVTCVVVDVKQEDPEVQF
NWYVDGVEVHNAKTKPREEEFNSTYRVVSVLTVLHQDWLNGKEYKCKVSN
KGLPSSIEKTISKAKGQPREPQVYTLPPSQEEMTKNQVSLTCDVSGFYPS
DIAVEWESDGQPENNYKTTPPVLDSDGSFFLYSRLTVDKSRWEEGDVFSC
SVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 scFv (VL-linker-VH) hIgG4 Variant 1
(SEQ ID NO: 3417)
QSALTQPRSASGNPGQRVTISCSGSSSNMGRRPVNWYQQIPGTAPKLLIYSQNQRPSGVPDRFSGSQSGTSA
SLTISGLQSEDEAEYFCAVWDDIGRVLQLGGGTQLAVLggggsggggsggggsEVQLVETGGGLIQPGRSLR
LSCAASGFTFSSYAMHWVRQAPGKGLEWVAVISYAGEVKYYADSVKGRFTISRDNSKNTLYLQMNSLRAEDT
AVYYCARDPLPLHYYGMDVWGQGTTVTVSSESKYGPPCPPCPAPPVA()GPSVFLFPPKPKDTLMISRTPEV
TCVVVDVKQEDPEVQFNWYVDGVEVHNAKTKPREEQFNSTYRVVSVLTVLHQDWLNGKEYKCKVSNKGLPSS
IEKTISKAKGQPREPQVYTLPPSQEQMTKNQVKLTCLVKGFYPSDIAVEWESNGQPENNYKTTPPVLDSDGS
FFLYSRLTVDKSRWQEGNVFSCSVMHEALHNHYTQKSLSLSPG;
CPA.9.086 scFv (VH-linker-VL) hIgG4 Variant 1
(SEQ ID NO: 3427)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSggggsggggsggggsQSALTQPR
SASGNPGQRVTISCSGSSSNMGRRPVNWYQQIPGTAPKLLIYSQNQRPSGVPDRFSGSQSGTSASLTISGLQ
SEDEAEYFCAVWDDIGRVLQLGGGTQLAVLESKYGPPCPPCPAPPVA()GPSVFLFPPKPKDTLMISRTPEV
TCVVVDVKQEDPEVQFNWYVDGVEVHNAKTKPREEQFNSTYRVVSVLTVLHQDWLNGKEYKCKVSNKGLPSS
IEKTISKAKGQPREPQVYTLPPSQEQMTKNQVKLTCLVKGFYPSDIAVEWESNGQPENNYKTTPPVLDSDGS
FFLYSRLTVDKSRWQEGNVFSCSVMHEALHNHYTQKSLSLSPG;
CPA.9.086 hIgG4 Variant 1
(SEQ ID NO: 3457)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASTKGPSVFPLAPCSRSTSESTA
ALGCLVKDYFPEPVTVSWNSGALTSGVHTFPAVLQSSGLYSLSSVVTVPSSSLGTKTYTCNVDHKPSDTKVD
KRVESKYGPPCPPCPAPPVA()GPSVFLFPPKPKDTLMISRTPEVTCVVVDVKQEDPEVQFNWYVDGVEVHN
AKTKPREEQFNSTYRVVSVLTVLHQDWLNGKEYKCKVSNKGLPSSIEKTISKAKGQPREPQVYTLPPSQEQM
TKNQVKLTCLVKGFYPSDIAVEWESNGQPENNYKTTPPVLDSDGSFFLYSRLTVDKSRWQEGNVFSCSVMHE
ALHNHYTQKSLSLSPG;
CHA.9.547.18 scFv (VL-linker-VH) hIgG4 Variant 1
(SEQ ID NO: 3517)
DIQMTQSPSSLSASVGDRITITCRASQSMAIWLSWYQQKPGKAPKLLIYKASKSHTGVPSRFSGSGSGTDFT
LTISSLQPEDFATYYCQQGQSYPYTFGQGTKLEIKggggsggggsggggsEVQLVESGGGLVQPGGSLRLSC
AASGFTFSSYIMSWVRQAPGKGLEWVATISGGSKGQYYADSVKGRFTISRDNSKNTLYLQMNSLRAEDTAVY
YCAKWLLSYYAMDYWGQGTLVTVSSESKYGPPCPPCPAPPVA()GPSVFLFPPKPKDTLMISRTPEVTCVVV
DVKQEDPEVQFNWYVDGVEVHNAKTKPREEQFNSTYRVVSVLTVLHQDWLNGKEYKCKVSNKGLPSSIEKTI
SKAKGQPREPQVYTLPPSQEQMTKNQVKLTCLVKGFYPSDIAVEWESNGQPENNYKTTPPVLDSDGSFFLYS
RLTVDKSRWQEGNVFSCSVMHEALHNHYTQKSLSLSPG;
CHA.9.547.18 scFv (VH-linker-VL) hIgG4 Variant 1
(SEQ ID NO: 3527)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSggggsggggsggggsDIQMTQSPSS
LSASVGDRITITCRASQSMAIWLSWYQQKPGKAPKLLIYKASKSHTGVPSRFSGSGSGTDFTLTISSLQPED
FATYYCQQGQSYPYTFGQGTKLEIKESKYGPPCPPCPAPPVA()GPSVFLFPPKPKDTLMISRTPEVTCVVV
DVKQEDPEVQFNWYVDGVEVHNAKTKPREEQFNSTYRVVSVLTVLHQDWLNGKEYKCKVSNKGLPSSIEKTI
SKAKGQPREPQVYTLPPSQEQMTKNQVKLTCLVKGFYPSDIAVEWESNGQPENNYKTTPPVLDSDGSFFLYS
RLTVDKSRWQEGNVFSCSVMHEALHNHYTQKSLSLSPG;
and
CHA.9.547.18 hIgG4 Variant 1
(SEQ ID NO: 3557)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSNTKVDKR
VESKYGPPCPPCPAPPVA()GPSVFLFPPKPKDTLMISRTPEVTCVVVDVKQEDPEVQFNWYVDGVEVHNAK
TKPREEQFNSTYRVVSVLTVLHQDWLNGKEYKCKVSNKGLPSSIEKTISKAKGQPREPQVYTLPPSQEQMTK
NQVKLTCLVKGFYPSDIAVEWESNGQPENNYKTTPPVLDSDGSFFLYSRLTVDKSRWQEGNVFSCSVMHEAL
HNHYTQKSLSLSPG;

In some embodiments, the anti-PVRIG portion of the anti-PVRIG/anti-TIGIT bispecific antibody comprises the following anti-PVRIG sequence

CHA.7.518.1 scFv (VH-linker-VL) hIgG4 Variant 2

(SEQ ID NO: 3247)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSggggsggggsggggsDIQMTQSPSSLSASV
GDRVTITCRVSENIYSNLAWYQQKPGKAPKLLIYEATNLAEGVPSRFSGS
GSGTDFTLTISSLQPEDFATYYCQHFWGTPYTFGQGTKLEIKESKYGPPC
PPCPAPPVA()GPSVFLFPPKPKDTLMISRTPEVTCVVVDVKQEDPEVQF
NWYVDGVEVHNAKTKPREEEFNSTYRVVSVLTVLHQDWLNGKEYKCKVSN
KGLPSSIEKTISKAKGQPREPQVYTLPPSQEEMTKNQVSLTCDVSGFYPS
DIAVEWESDGQPENNYKTTPPVLDSDGSFFLYSRLTVDKSRWEEGDVFSC
SVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 scFv (VL-linker-VH) hIgG4 Variant 1
(SEQ ID NO: 3417)
QSALTQPRSASGNPGQRVTISCSGSSSNMGRRPVNWYQQIPGTAPKLLIYSQNQRPSGVPDRFSGSQSGTSA
SLTISGLQSEDEAEYFCAVWDDIGRVLQLGGGTQLAVLggggsggggsggggsEVQLVETGGGLIQPGRSLR
LSCAASGFTFSSYAMHWVRQAPGKGLEWVAVISYAGEVKYYADSVKGRFTISRDNSKNTLYLQMNSLRAEDT
AVYYCARDPLPLHYYGMDVWGQGTTVTVSSESKYGPPCPPCPAPPVA()GPSVFLFPPKPKDTLMISRTPEV
TCVVVDVKQEDPEVQFNWYVDGVEVHNAKTKPREEQFNSTYRVVSVLTVLHQDWLNGKEYKCKVSNKGLPSS
IEKTISKAKGQPREPQVYTLPPSQEQMTKNQVKLTCLVKGFYPSDIAVEWESNGQPENNYKTTPPVLDSDGS
FFLYSRLTVDKSRWQEGNVFSCSVMHEALHNHYTQKSLSLSPG;
CPA.9.086 scFv (VH-linker-VL) hIgG4 Variant 1
(SEQ ID NO: 3427)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSggggsggggsggggsQSALTQPR
SASGNPGQRVTISCSGSSSNMGRRPVNWYQQIPGTAPKLLIYSQNQRPSGVPDRFSGSQSGTSASLTISGLQ
SEDEAEYFCAVWDDIGRVLQLGGGTQLAVLESKYGPPCPPCPAPPVA()GPSVFLFPPKPKDTLMISRTPEV
TCVVVDVKQEDPEVQFNWYVDGVEVHNAKTKPREEQFNSTYRVVSVLTVLHQDWLNGKEYKCKVSNKGLPSS
IEKTISKAKGQPREPQVYTLPPSQEQMTKNQVKLTCLVKGFYPSDIAVEWESNGQPENNYKTTPPVLDSDGS
FFLYSRLTVDKSRWQEGNVFSCSVMHEALHNHYTQKSLSLSPG;
CPA.9.086 hIgG4 Variant 1
(SEQ ID NO: 3457)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASTKGPSVFPLAPCSRSTSESTA
ALGCLVKDYFPEPVTVSWNSGALTSGVHTFPAVLQSSGLYSLSSVVTVPSSSLGTKTYTCNVDHKPSDTKVD
KRVESKYGPPCPPCPAPPVA()GPSVFLFPPKPKDTLMISRTPEVTCVVVDVKQEDPEVQFNWYVDGVEVHN
AKTKPREEQFNSTYRVVSVLTVLHQDWLNGKEYKCKVSNKGLPSSIEKTISKAKGQPREPQVYTLPPSQEQM
TKNQVKLTCLVKGFYPSDIAVEWESNGQPENNYKTTPPVLDSDGSFFLYSRLTVDKSRWQEGNVFSCSVMHE
ALHNHYTQKSLSLSPG;
CHA.9.547.18 scFv (VL-linker-VH) hIgG4 Variant 1
(SEQ ID NO: 3517)
DIQMTQSPSSLSASVGDRITITCRASQSMAIWLSWYQQKPGKAPKLLIYKASKSHTGVPSRFSGSGSGTDFT
LTISSLQPEDFATYYCQQGQSYPYTFGQGTKLEIKggggsggggsggggsEVQLVESGGGLVQPGGSLRLSC
AASGFTFSSYIMSWVRQAPGKGLEWVATISGGSKGQYYADSVKGRFTISRDNSKNTLYLQMNSLRAEDTAVY
YCAKWLLSYYAMDYWGQGTLVTVSSESKYGPPCPPCPAPPVA()GPSVFLFPPKPKDTLMISRTPEVTCVVV
DVKQEDPEVQFNWYVDGVEVHNAKTKPREEQFNSTYRVVSVLTVLHQDWLNGKEYKCKVSNKGLPSSIEKTI
SKAKGQPREPQVYTLPPSQEQMTKNQVKLTCLVKGFYPSDIAVEWESNGQPENNYKTTPPVLDSDGSFFLYS
RLTVDKSRWQEGNVFSCSVMHEALHNHYTQKSLSLSPG;
CHA.9.547.18 scFv (VH-linker-VL) hIgG4 Variant 1
(SEQ ID NO: 3527)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSggggsggggsggggsDIQMTQSPSS
LSASVGDRITITCRASQSMAIWLSWYQQKPGKAPKLLIYKASKSHTGVPSRFSGSGSGTDFTLTISSLQPED
FATYYCQQGQSYPYTFGQGTKLEIKESKYGPPCPPCPAPPVA()GPSVFLFPPKPKDTLMISRTPEVTCVVV
DVKQEDPEVQFNWYVDGVEVHNAKTKPREEQFNSTYRVVSVLTVLHQDWLNGKEYKCKVSNKGLPSSIEKTI
SKAKGQPREPQVYTLPPSQEQMTKNQVKLTCLVKGFYPSDIAVEWESNGQPENNYKTTPPVLDSDGSFFLYS
RLTVDKSRWQEGNVFSCSVMHEALHNHYTQKSLSLSPG;
and
CHA.9.547.18 hIgG4 Variant 1
(SEQ ID NO: 3557)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSNTKVDKR
VESKYGPPCPPCPAPPVA()GPSVFLFPPKPKDTLMISRTPEVTCVVVDVKQEDPEVQFNWYVDGVEVHNAK
TKPREEQFNSTYRVVSVLTVLHQDWLNGKEYKCKVSNKGLPSSIEKTISKAKGQPREPQVYTLPPSQEQMTK
NQVKLTCLVKGFYPSDIAVEWESNGQPENNYKTTPPVLDSDGSFFLYSRLTVDKSRWQEGNVFSCSVMHEAL
HNHYTQKSLSLSPG.

In some embodiments, the anti-PVRIG portion of the anti-PVRIG/anti-TIGIT bispecific antibody comprises the following anti-PVRIG sequence

CHA.7.518.1 hIgG4 Variant 1

(SEQ ID NO: 3257)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPPVA()GPSVFLFPPKPKD
TLMISRTPEVTCVVVDVKQEDPEVQFNWYVDGVEVHNAKTKPREEQFNST
YRVVSVLTVLHQDWLNGKEYKCKVSNKGLPSSIEKTISKAKGQPREPQVY
TLPPSQEQMTKNQVKLTCLVKGFYPSDIAVEWESNGQPENNYKTTPPVLD
SDGSFFLYSRLTVDKSRWQEGNVFSCSVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 scFv (VL-linker-VH) hIgG4 Variant 2
(SEQ ID NO: 3437)
QSALTQPRSASGNPGQRVTISCSGSSSNMGRRPVNWYQQIPGTAPKLLIYSQNQRPSGVPDRFSGSQSGTSA
SLTISGLQSEDEAEYFCAVWDDIGRVLQLGGGTQLAVLggggsggggsggggsEVQLVETGGGLIQPGRSLR
LSCAASGFTFSSYAMHWVRQAPGKGLEWVAVISYAGEVKYYADSVKGRFTISRDNSKNTLYLQMNSLRAEDT
AVYYCARDPLPLHYYGMDVWGQGTTVTVSSESKYGPPCPPCPAPPVA()GPSVFLFPPKPKDTLMISRTPEV
TCVVVDVKQEDPEVQFNWYVDGVEVHNAKTKPREEEFNSTYRVVSVLTVLHQDWLNGKEYKCKVSNKGLPSS
IEKTISKAKGQPREPQVYTLPPSQEEMTKNQVSLTCDVSGFYPSDIAVEWESDGQPENNYKTTPPVLDSDGS
FFLYSRLTVDKSRWEEGDVFSCSVMHEALHNHYTQKSLSLSPG;
CPA.9.086 scFv (VH-linker-VL) hIgG4 Variant 2
(SEQ ID NO: 3447)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSggggsggggsggggsQSALTQPR
SASGNPGQRVTISCSGSSSNMGRRPVNWYQQIPGTAPKLLIYSQNQRPSGVPDRFSGSQSGTSASLTISGLQ
SEDEAEYFCAVWDDIGRVLQLGGGTQLAVLESKYGPPCPPCPAPPVA()GPSVFLFPPKPKDTLMISRTPEV
TCVVVDVKQEDPEVQFNWYVDGVEVHNAKTKPREEEFNSTYRVVSVLTVLHQDWLNGKEYKCKVSNKGLPSS
IEKTISKAKGQPREPQVYTLPPSQEEMTKNQVSLTCDVSGFYPSDIAVEWESDGQPENNYKTTPPVLDSDGS
FFLYSRLTVDKSRWEEGDVFSCSVMHEALHNHYTQKSLSLSPG;
CPA.9.086 hIgG4 Variant 2
(SEQ ID NO: 3467)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASTKGPSVFPLAPCSRSTSESTA
ALGCLVKDYFPEPVTVSWNSGALTSGVHTFPAVLQSSGLYSLSSVVTVPSSSLGTKTYTCNVDHKPSDTKVD
KRVESKYGPPCPPCPAPPVA()GPSVFLFPPKPKDTLMISRTPEVTCVVVDVKQEDPEVQFNWYVDGVEVHN
AKTKPREEEFNSTYRVVSVLTVLHQDWLNGKEYKCKVSNKGLPSSIEKTISKAKGQPREPQVYTLPPSQEEM
TKNQVSLTCDVSGFYPSDIAVEWESDGQPENNYKTTPPVLDSDGSFFLYSRLTVDKSRWEEGDVFSCSVMHE
ALHNHYTQKSLSLSPG;
CHA.9.547.18 scFv (VL-linker-VH) hIgG4 Variant 2
(SEQ ID NO: 3537)
DIQMTQSPSSLSASVGDRITITCRASQSMAIWLSWYQQKPGKAPKLLIYKASKSHTGVPSRFSGSGSGTDFT
LTISSLQPEDFATYYCQQGQSYPYTFGQGTKLEIKggggsggggsggggsEVQLVESGGGLVQPGGSLRLSC
AASGFTFSSYIMSWVRQAPGKGLEWVATISGGSKGQYYADSVKGRFTISRDNSKNTLYLQMNSLRAEDTAVY
YCAKWLLSYYAMDYWGQGTLVTVSSESKYGPPCPPCPAPPVA()GPSVFLFPPKPKDTLMISRTPEVTCVVV
DVKQEDPEVQFNWYVDGVEVHNAKTKPREEEFNSTYRVVSVLTVLHQDWLNGKEYKCKVSNKGLPSSIEKTI
SKAKGQPREPQVYTLPPSQEEMTKNQVSLTCDVSGFYPSDIAVEWESDGQPENNYKTTPPVLDSDGSFFLYS
RLTVDKSRWEEGDVFSCSVMHEALHNHYTQKSLSLSPG;
CHA.9.547.18 scFv (VH-linker-VL) hIgG4 Variant 2
(SEQ ID NO: 3547)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSggggsggggsggggsDIQMTQSPSS
LSASVGDRITITCRASQSMAIWLSWYQQKPGKAPKLLIYKASKSHTGVPSRFSGSGSGTDFTLTISSLQPED
FATYYCQQGQSYPYTFGQGTKLEIKESKYGPPCPPCPAPPVA()GPSVFLFPPKPKDTLMISRTPEVTCVVV
DVKQEDPEVQFNWYVDGVEVHNAKTKPREEEFNSTYRVVSVLTVLHQDWLNGKEYKCKVSNKGLPSSIEKTI
SKAKGQPREPQVYTLPPSQEEMTKNQVSLTCDVSGFYPSDIAVEWESDGQPENNYKTTPPVLDSDGSFFLYS
RLTVDKSRWEEGDVFSCSVMHEALHNHYTQKSLSLSPG;
and
CHA.9.547.18 hIgG4 Variant 2
(SEQ ID NO: 3567)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSDTKVDKR
VESKYGPPCPPCPAPPVA()GPSVFLFPPKPKDTLMISRTPEVTCVVVDVKQEDPEVQFNWYVDGVEVHNAK
TKPREEEFNSTYRVVSVLTVLHQDWLNGKEYKCKVSNKGLPSSIEKTISKAKGQPREPQVYTLPPSQEEMTK
NQVSLTCDVSGFYPSDIAVEWESDGQPENNYKTTPPVLDSDGSFFLYSRLTVDKSRWEEGDVFSCSVMHEAL
HNHYTQKSLSLSPG.

In some embodiments, the anti-PVRIG portion of the anti-PVRIG/anti-TIGIT bispecific antibody comprises the following anti-PVRIG sequence

CHA.7.518.1 hIgG4 Variant 2

(SEQ ID NO: 3267)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSDTKVDKRVESKYGPPCPPCPAPPVA()GPSVFLFPPKPKD
TLMISRTPEVTCVVVDVKQEDPEVQFNWYVDGVEVHNAKTKPREEEFNST
YRVVSVLTVLHQDWLNGKEYKCKVSNKGLPSSIEKTISKAKGQPREPQVY
TLPPSQEEMTKNQVSLTCDVSGFYPSDIAVEWESDGQPENNYKTTPPVLD
SDGSFFLYSRLTVDKSRWEEGDVFSCSVMHEALHNHYTQKSLSLSPG;

and the anti-TIGIT portion of the anti-PVRIG/anti-TIGIT bispecific antibody comprises an anti-TIGIT sequence selected from the following:

CPA.9.086 scFv (VL-linker-VH) hIgG4 Variant 1

(SEQ ID NO: 3417)

QSALTQPRSASGNPGQRVTISCSGSSSNMGRRPVNWYQQIPGTAPKLLIY
SQNQRPSGVPDRFSGSQSGTSASLTISGLQSEDEAEYFCAVWDDIGRVLQ
LGGGTQLAVLggggsggggsggggsEVQLVETGGGLIQPGRSLRLSCAAS
GFTFSSYAMHWVRQAPGKGLEWVAVISYAGEVKYYADSVKGRFTISRDNS
KNTLYLQMNSLRAEDTAVYYCARDPLPLHYYGMDVWGQGTTVTVSSESKY
GPPCPPCPAPPVA()GPSVFLFPPKPKDTLMISRTPEVTCVVVDVKQEDP
EVQFNWYVDGVEVHNAKTKPREEQFNSTYRVVSVLTVLHQDWLNGKEYKC
KVSNKGLPSSIEKTISKAKGQPREPQVYTLPPSQEQMTKNQVKLTCLVKG
FYPSDIAVEWESNGQPENNYKTTPPVLDSDGSFFLYSRLTVDKSRWQEGN
VFSCSVMHEALHNHYTQKSLSLSPG;
CPA.9.086 scFv (VH-linker-VL) hIgG4 Variant 1

(SEQ ID NO: 3427)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSggggsggggsggggsQSALTQPRSASGNP
GQRVTISCSGSSSNMGRRPVNWYQQIPGTAPKLLIYSQNQRPSGVPDRFS
GSQSGTSASLTISGLQSEDEAEYFCAVWDDIGRVLQLGGGTQLAVLESKY
GPPCPPCPAPPVA()GPSVFLFPPKPKDTLMISRTPEVTCVVVDVKQEDP
EVQFNWYVDGVEVHNAKTKPREEQFNSTYRVVSVLTVLHQDWLNGKEYKC
KVSNKGLPSSIEKTISKAKGQPREPQVYTLPPSQEQMTKNQVKLTCLVKG
FYPSDIAVEWESNGQPENNYKTTPPVLDSDGSFFLYSRLTVDKSRWQEGN
VFSCSVMHEALHNHYTQKSLSLSPG;
CPA.9.086 hIgG4 Variant 1

(SEQ ID NO: 3457)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSDTKVDKRVESKYGPPCPPCPAPPVA()GPSVFLFPPKPK
DTLMISRTPEVTCVVVDVKQEDPEVQFNWYVDGVEVHNAKTKPREEQFNS
TYRVVSVLTVLHQDWLNGKEYKCKVSNKGLPSSIEKTISKAKGQPREPQV
YTLPPSQEQMTKNQVKLTCLVKGFYPSDIAVEWESNGQPENNYKTTPPVL
DSDGSFFLYSRLTVDKSRWQEGNVFSCSVMHEALHNHYTQKSLSLSPG;
CHA.9.547.18 scFv (VL-linker-VH) hIgG4 Variant 1

(SEQ ID NO: 3517)

DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIKggggsggggsggggsEVQLVESGGGLVQPGGSLRLSCAASGFT
FSSYIMSWVRQAPGKGLEWVATISGGSKGQYYADSVKGRFTISRDNSKNT
LYLQMNSLRAEDTAVYYCAKWLLSYYAMDYWGQGTLVTVSSESKYGPPCP
PCPAPPVA()GPSVFLFPPKPKDTLMISRTPEVTCVVVDVKQEDPEVQFN
WYVDGVEVHNAKTKPREEQFNSTYRVVSVLTVLHQDWLNGKEYKCKVSNK
GLPSSIEKTISKAKGQPREPQVYTLPPSQEQMTKNQVKLTCLVKGFYPSD
IAVEWESNGQPENNYKTTPPVLDSDGSFFLYSRLTVDKSRWQEGNVFSCS
VMHEALHNHYTQKSLSLSPG;
CHA.9.547.18 scFv (VH-linker-VL) hIgG4 Variant 1

(SEQ ID NO: 3527)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSSggggsggggsggggsDIQMTQSPSSLSASVG
DRITITCRASQSMAIWLSWYQQKPGKAPKLLIYKASKSHTGVPSRFSGSG
SGTDFTLTISSLQPEDFATYYCQQGQSYPYTFGQGTKLEIKESKYGPPCP
PCPAPPVA()GPSVFLFPPKPKDTLMISRTPEVTCVVVDVKQEDPEVQFN
WYVDGVEVHNAKTKPREEQFNSTYRVVSVLTVLHQDWLNGKEYKCKVSNK
GLPSSIEKTISKAKGQPREPQVYTLPPSQEQMTKNQVKLTCLVKGFYPSD
IAVEWESNGQPENNYKTTPPVLDSDGSFFLYSRLTVDKSRWQEGNVFSCS
VMHEALHNHYTQKSLSLSPG;
and
CHA.9.547.18 hIgG4 Variant 1

(SEQ ID NO: 3557)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPPVA()GPSVFLFPPKPKDT
LMISRTPEVTCVVVDVKQEDPEVQFNWYVDGVEVHNAKTKPREEQFNSTY
RVVSVLTVLHQDWLNGKEYKCKVSNKGLPSSIEKTISKAKGQPREPQVYT
LPPSQEQMTKNQVKLTCLVKGFYPSDIAVEWESNGQPENNYKTTPPVLDS
DGSFFLYSRLTVDKSRWQEGNVFSCSVMHEALHNHYTQKSLSLSPG.

In some embodiments, the anti-PVRIG portion of the anti-PVRIG/anti-TIGIT bispecific antibody comprises the following anti-PVRIG sequence

CHA.7.518.1 VH Clambda-VL-CH1 Crossmab hIgG4
Fc Y349C E356D L358L T366S L368A Y407V Hole

(SEQ ID NO: 3655)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASVAAPSVFIFPPSDEQLKSGTASVVCLLN
NFYPREAKVQWKVDNALQSGNSQESVTEQDSKDSTYSLSSTLTLSKADYE
KHKVYACEVTHQGLSSPVTKSFNRGECESKYGPPCPPCPAPEFLGGPSVF
LFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKG
QPREPQVCTLPPSQDELTKNQVSLSCAVKGFYPSDIAVEWESNGQPENNY
KTTPPVLDSDGSFFLVSRLTVDKSRWQEGNVFSCSVMHEALHNHYTQKSL
SLSPG;

and the anti-TIGIT portion of the anti-PVRIG/anti-TIGIT bispecific antibody comprises an anti-TIGIT sequence selected from the group consisting of the following sequences:

CPA.9.086 VH Clambda-VL-CH1 Crossmab hIgG4 Fc
Y354C E356D M348L T366W Knob

(SEQ ID NO: 3487)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSASGQPKAAPSVTLFPPSSEELQANKATLV
CLISDFYPGAVTVAWKADSSPVKAGVETTTPSKQSNNKYAASSYLSLTPE
QWKSHRSYSCQVTHEGSTVEKTVAPTECSESKYGPPCPPCPAPEFLGGPS
VFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKA
KGQPREPQVYTLPPCQDELTKNQVSLWCLVKGFYPSDIAVEWESNGQPEN
NYKTTPPVLDSDGSFFLYSRLTVDKSRWQEGNVFSCSVMHEALHNHYTQK
SLSLSPG;
CPA.9.086 VH hIgG4 Fc Y354C E356D M348L T366W
Knob

(SEQ ID NO: 3507)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSASASTKGPSVFPLAPCSRSTSESTAALGC
LVKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQ
VYTLPPCQDELTKNQVSLWCLVKGFYPSDIAVEWESNGQPENNYKTTPPV
LDSDGSFFLYSRLTVDKSRWQEGNVFSCSVMHEALHNHYTQKSLSLSPG;
CHA.9.547.18 VH Clambda-VL-CH1 Crossmab hIgG4
Fc Y354C E356D M348L T366W Knob

(SEQ ID NO: 3656)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSSASVAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGECESKYGPPCPPCPAPEFLGGPSVFL
FPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQ
PREPQVYTLPPCQDELTKNQVSLWCLVKGFYPSDIAVEWESNGQPENNYK
TTPPVLDSDGSFFLYSRLTVDKSRWQEGNVFSCSVMHEALHNHYTQKSLS
LSPG;
and
CHA.9.547.18 VH hIgG4 Fc Y354C E356D M348L
T366W Knob

(SEQ ID NO: 3607)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSSASASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPCQDELTKNQVSLWCLVKGFYPSDIAVEWESNGQPENNYKTTPPVLD
SDGSFFLYSRLTVDKSRWQEGNVFSCSVMHEALHNHYTQKSLSLSPG.

In some embodiments, the anti-PVRIG portion of the anti-PVRIG/anti-TIGIT bispecific antibody comprises the following anti-PVRIG sequence

CHA.7.518.1 VH Clambda-VL-CH1 Crossmab hIgG4
Fc Y354C E356D M348L T366W Knob

(SEQ ID NO: 3657)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASVAAPSVFIFPPSDEQLKSGTASVVCLLN
NFYPREAKVQWKVDNALQSGNSQESVTEQDSKDSTYSLSSTLTLSKADYE
KHKVYACEVTHQGLSSPVTKSFNRGECESKYGPPCPPCPAPEFLGGPSVF
LFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKG
QPREPQVYTLPPCQDELTKNQVSLWCLVKGFYPSDIAVEWESNGQPENNY
KTTPPVLDSDGSFFLYSRLTVDKSRWQEGNVFSCSVMHEALHNHYTQKSL
SLSPG;

and the anti-TIGIT portion of the anti-PVRIG/anti-TIGIT bispecific antibody comprises an anti-TIGIT sequence selected from the group consisting of the following sequences:

CPA.9.086 VH Clambda-VL-CH1 Crossmab hIgG4 Fc
Y349C E356D L358L T366S L368A Y407V Hole

(SEQ ID NO: 3477)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSASGQPKAAPSVTLFPPSSEELQANKATLV
CLISDFYPGAVTVAWKADSSPVKAGVETTTPSKQSNNKYAASSYLSLTPE
QWKSHRSYSCQVTHEGSTVEKTVAPTECSESKYGPPCPPCPAPEFLGGPS
VFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKA
KGQPREPQVCTLPPSQDELTKNQVSLSCAVKGFYPSDIAVEWESNGQPEN
NYKTTPPVLDSDGSFFLVSRLTVDKSRWQEGNVFSCSVMHEALHNHYTQK
SLSLSPG;
CPA.9.086 VH hIgG4 Fc Y349C E356D L358L T366S
L368A Y407V Hole

(SEQ ID NO: 3497)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSASASTKGPSVFPLAPCSRSTSESTAALGC
LVKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQ
VCTLPPSQDELTKNQVSLSCAVKGFYPSDIAVEWESNGQPENNYKTTPPV
LDSDGSFFLVSRLTVDKSRWQEGNVFSCSVMHEALHNHYTQKSLSLSPG;
CHA.9.547.18 VH Clambda-VL-CH1 Crossmab hIgG4
Fc Y349C E356D L358L T366S L368A Y407V Hole

(SEQ ID NO: 3658)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSSASVAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGECESKYGPPCPPCPAPEFLGGPSVFL
FPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQ
PREPQVCTLPPSQDELTKNQVSLSCAVKGFYPSDIAVEWESNGQPENNYK
TTPPVLDSDGSFFLVSRLTVDKSRWQEGNVFSCSVMHEALHNHYTQKSLS
LSPG;
and
CHA.9.547.18 VH hIgG4 Fc Y349C E356D L358L
T366S L368A Y407V Hole

(SEQ ID NO: 3597)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSSASASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVC
TLPPSQDELTKNQVSLSCAVKGFYPSDIAVEWESNGQPENNYKTTPPVLD
SDGSFFLVSRLTVDKSRWQEGNVFSCSVMHEALHNHYTQKSLSLSPG.

In some embodiments, the anti-PVRIG portion of the anti-PVRIG/anti-TIGIT bispecific antibody comprises the following anti-PVRIG sequence

CHA.7.518.1 VH hIgG4 Fc Y349C E356D L358L
T366S L368A Y407V Hole

(SEQ ID NO: 3297)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASASTKGPSVFPLAPCSRSTSESTAALGCL
VKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPK
DTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQV
CTLPPSQDELTKNQVSLSCAVKGFYPSDIAVEWESNGQPENNYKTTPPVL
DSDGSFFLVSRLTVDKSRWQEGNVFSCSVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 VH Clambda-VL-CH1 Crossmab hIgG4
Fc Y354C E356D M348L T366W Knob

(SEQ ID NO: 3487)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSASGQPKAAPSVTLFPPSSEELQANKATLV
CLISDFYPGAVTVAWKADSSPVKAGVETTTPSKQSNNKYAASSYLSLTPE
QWKSHRSYSCQVTHEGSTVEKTVAPTECSESKYGPPCPPCPAPEFLGGPS
VFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKA
KGQPREPQVYTLPPCQDELTKNQVSLWCLVKGFYPSDIAVEWESNGQPEN
NYKTTPPVLDSDGSFFLYSRLTVDKSRWQEGNVFSCSVMHEALHNHYTQK
SLSLSPG;
CPA.9.086 VH hIgG4 Fc Y354C E356D M348L
T366W Knob

(SEQ ID NO: 3507)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSASASTKGPSVFPLAPCSRSTSESTAALGC
LVKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQ
VYTLPPCQDELTKNQVSLWCLVKGFYPSDIAVEWESNGQPENNYKTTPPV
LDSDGSFFLYSRLTVDKSRWQEGNVFSCSVMHEALHNHYTQKSLSLSPG;
CHA.9.547.18 VH Clambda-VL-CH1 Crossmab
hIgG4 Fc Y354C E356D M348L T366W Knob

(SEQ ID NO: 3659)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSSASVAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGECESKYGPPCPPCPAPEFLGGPSVFL
FPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQ
PREPQVYTLPPCQDELTKNQVSLWCLVKGFYPSDIAVEWESNGQPENNYK
TTPPVLDSDGSFFLYSRLTVDKSRWQEGNVFSCSVMHEALHNHYTQKSLS
LSPG;
and
CHA.9.547.18 VH hIgG4 Fc Y354C E356D M348L
T366W Knob

(SEQ ID NO: 3607)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSSASASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPCQDELTKNQVSLWCLVKGFYPSDIAVEWESNGQPENNYKTTPPVLD
SDGSFFLYSRLTVDKSRWQEGNVFSCSVMHEALHNHYTQKSLSLSPG.

In some embodiments, the anti-PVRIG portion of the anti-PVRIG/anti-TIGIT bispecific antibody comprises the following anti-PVRIG sequence

CHA.7.518.1 VH hIgG4 Fc Y354C E356D M348L
T366W Knob

(SEQ ID NO: 3307)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASASTKGPSVFPLAPCSRSTSESTAALGCL
VKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPK
DTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQV
YTLPPCQDELTKNQVSLWCLVKGFYPSDIAVEWESNGQPENNYKTTPPVL
DSDGSFFLYSRLTVDKSRWQEGNVFSCSVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 VH Clambda-VL-CH1 Crossmab hIgG4
Fc Y349C E356D L358L T366S L368A Y407V Hole

(SEQ ID NO: 3477)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSASGQPKAAPSVTLFPPSSEELQANKATLV
CLISDFYPGAVTVAWKADSSPVKAGVETTTPSKQSNNKYAASSYLSLTPE
QWKSHRSYSCQVTHEGSTVEKTVAPTECSESKYGPPCPPCPAPEFLGGPS
VFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKA
KGQPREPQVCTLPPSQDELTKNQVSLSCAVKGFYPSDIAVEWESNGQPEN
NYKTTPPVLDSDGSFFLVSRLTVDKSRWQEGNVFSCSVMHEALHNHYTQK
SLSLSPG;
CPA.9.086 VH hIgG4 Fc Y349C E356D L358L
T366S L368A Y407V Hole

(SEQ ID NO: 3497)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSASASTKGPSVFPLAPCSRSTSESTAALGC
LVKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQ
VCTLPPSQDELTKNQVSLSCAVKGFYPSDIAVEWESNGQPENNYKTTPPV
LDSDGSFFLVSRLTVDKSRWQEGNVFSCSVMHEALHNHYTQKSLSLSPG;
CHA.9.547.18 VH Clambda-VL-CH1 Crossmab hIgG4
Fc Y349C E356D L358L T366S L368A Y407V Hole

(SEQ ID NO: 3660)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSSASVAAPSVFIFPPSDEQLKSGTASVVCLLNN
FYPREAKVQWKVDNALQSGNSQESVTEQDSKDSTYSLSSTLTLSKADYEK
HKVYACEVTHQGLSSPVTKSFNRGECESKYGPPCPPCPAPEFLGGPSVFL
FPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQ
PREPQVCTLPPSQDELTKNQVSLSCAVKGFYPSDIAVEWESNGQPENNYK
TTPPVLDSDGSFFLVSRLTVDKSRWQEGNVFSCSVMHEALHNHYTQKSLS
LSPG;
and
CHA.9.547.18 VH hIgG4 Fc Y349C E356D L358L
T366S L368A Y407V Hole

(SEQ ID NO: 3597)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSSASASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVC
TLPPSQDELTKNQVSLSCAVKGFYPSDIAVEWESNGQPENNYKTTPPVLD
SDGSFFLVSRLTVDKSRWQEGNVFSCSVMHEALHNHYTQKSLSLSPG.

In some embodiments, the anti-PVRIG portion of the anti-PVRIG/anti-TIGIT bispecific antibody comprises the following anti-PVRIG sequence

CHA.7.518.4 scFv (VL-linker-VH) hIgG4 Variant 1

(SEQ ID NO: 3317)

DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIKggggsggggsggggsQVQLVQSGAEVKKPGASVKVSCKASGYT
FTDYNINWVRQAPGQGLEWMGYIYPYIGGSGYAQKFQGRVTMTRDTSTST
VYMELSSLRSEDTAVYYCAREDKTARNAMDYWGQGTLVTVSSESKYGPPC
PPCPAPPVA()GPSVFLFPPKPKDTLMISRTPEVTCVVVDVKQEDPEVQF
NWYVDGVEVHNAKTKPREEQFNSTYRVVSVLTVLHQDWLNGKEYKCKVSN
KGLPSSIEKTISKAKGQPREPQVYTLPPSQEQMTKNQVKLTCLVKGFYPS
DIAVEWESNGQPENNYKTTPPVLDSDGSFFLYSRLTVDKSRWQEGNVFSC
SVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 scFv (VL-linker-VH) hIgG4 Variant 2

(SEQ ID NO: 3437)

QSALTQPRSASGNPGQRVTISCSGSSSNMGRRPVNWYQQIPGTAPKLLIY
SQNQRPSGVPDRFSGSQSGTSASLTISGLQSEDEAEYFCAVWDDIGRVLQ
LGGGTQLAVLggggsggggsggggsEVQLVETGGGLIQPGRSLRLSCAAS
GFTFSSYAMHWVRQAPGKGLEWVAVISYAGEVKYYADSVKGRFTISRDNS
KNTLYLQMNSLRAEDTAVYYCARDPLPLHYYGMDVWGQGTTVTVSSESKY
GPPCPPCPAPPVA()GPSVFLFPPKPKDTLMISRTPEVTCVVVDVKQEDP
EVQFNWYVDGVEVHNAKTKPREEEFNSTYRVVSVLTVLHQDWLNGKEYKC
KVSNKGLPSSIEKTISKAKGQPREPQVYTLPPSQEEMTKNQVSLTCDVSG
FYPSDIAVEWESDGQPENNYKTTPPVLDSDGSFFLYSRLTVDKSRWEEGD
VFSCSVMHEALHNHYTQKSLSLSPG;
CPA.9.086 scFv (VH-linker-VL) hIgG4 Variant 2

(SEQ ID NO: 3447)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSggggsggggsggggsQSALTQPRSASGNP
GQRVTISCSGSSSNMGRRPVNWYQQIPGTAPKLLIYSQNQRPSGVPDRFS
GSQSGTSASLTISGLQSEDEAEYFCAVWDDIGRVLQLGGGTQLAVLESKY
GPPCPPCPAPPVA()GPSVFLFPPKPKDTLMISRTPEVTCVVVDVKQEDP
EVQFNWYVDGVEVHNAKTKPREEEFNSTYRVVSVLTVLHQDWLNGKEYKC
KVSNKGLPSSIEKTISKAKGQPREPQVYTLPPSQEEMTKNQVSLTCDVSG
FYPSDIAVEWESDGQPENNYKTTPPVLDSDGSFFLYSRLTVDKSRWEEGD
VFSCSVMHEALHNHYTQKSLSLSPG;
CPA.9.086 hIgG4 Variant 2

(SEQ ID NO: 3467)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSDTKVDKRVESKYGPPCPPCPAPPVA()GPSVFLFPPKPK
DTLMISRTPEVTCVVVDVKQEDPEVQFNWYVDGVEVHNAKTKPREEEFNS
TYRVVSVLTVLHQDWLNGKEYKCKVSNKGLPSSIEKTISKAKGQPREPQV
YTLPPSQEEMTKNQVSLTCDVSGFYPSDIAVEWESDGQPENNYKTTPPVL
DSDGSFFLYSRLTVDKSRWEEGDVFSCSVMHEALHNHYTQKSLSLSPG;
CHA.9.547.18 scFv (VL-linker-VH) hIgG4 Variant 2

(SEQ ID NO: 3537)

DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIKggggsggggsggggsEVQLVESGGGLVQPGGSLRLSCAASGFT
FSSYIMSWVRQAPGKGLEWVATISGGSKGQYYADSVKGRFTISRDNSKNT
LYLQMNSLRAEDTAVYYCAKWLLSYYAMDYWGQGTLVTVSSESKYGPPCP
PCPAPPVA()GPSVFLFPPKPKDTLMISRTPEVTCVVVDVKQEDPEVQFN
WYVDGVEVHNAKTKPREEEFNSTYRVVSVLTVLHQDWLNGKEYKCKVSNK
GLPSSIEKTISKAKGQPREPQVYTLPPSQEEMTKNQVSLTCDVSGFYPSD
IAVEWESDGQPENNYKTTPPVLDSDGSFFLYSRLTVDKSRWEEGDVFSCS
VMHEALHNHYTQKSLSLSPG;
CHA.9.547.18 scFv (VH-linker-VL) hIgG4 Variant 2

(SEQ ID NO: 3547)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSSggggsggggsggggsDIQMTQSPSSLSASVG
DRITITCRASQSMAIWLSWYQQKPGKAPKLLIYKASKSHTGVPSRFSGSG
SGTDFTLTISSLQPEDFATYYCQQGQSYPYTFGQGTKLEIKESKYGPPCP
PCPAPPVA()GPSVFLFPPKPKDTLMISRTPEVTCVVVDVKQEDPEVQFN
WYVDGVEVHNAKTKPREEEFNSTYRVVSVLTVLHQDWLNGKEYKCKVSNK
GLPSSIEKTISKAKGQPREPQVYTLPPSQEEMTKNQVSLTCDVSGFYPSD
IAVEWESDGQPENNYKTTPPVLDSDGSFFLYSRLTVDKSRWEEGDVFSCS
VMHEALHNHYTQKSLSLSPG;
and
CHA.9.547.18 hIgG4 Variant 2

(SEQ ID NO: 3567)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSDTKVDKRVESKYGPPCPPCPAPPVA()GPSVFLFPPKPKDT
LMISRTPEVTCVVVDVKQEDPEVQFNWYVDGVEVHNAKTKPREEEFNSTY
RVVSVLTVLHQDWLNGKEYKCKVSNKGLPSSIEKTISKAKGQPREPQVYT
LPPSQEEMTKNQVSLTCDVSGFYPSDIAVEWESDGQPENNYKTTPPVLDS
DGSFFLYSRLTVDKSRWEEGDVFSCSVMHEALHNHYTQKSLSLSPG.

In some embodiments, the anti-PVRIG portion of the anti-PVRIG/anti-TIGIT bispecific antibody comprises the following anti-PVRIG sequence

CHA.7.518.4 scFv (VH-linker-VL) hIgG4 Variant 1

(SEQ ID NO: 3327)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSggggsggggsggggsDIQMTQSPSSLSASV
GDRVTITCRVSENIYDVLAWYQQKPGKAPKLLIYEATNLAEGVPSRFSGS
GSGTDFTLTISSLQPEDFATYYCQHFWGTPYTFGQGTKLEIKESKYGPPC
PPCPAPPVA()GPSVFLFPPKPKDTLMISRTPEVTCVVVDVKQEDPEVQF
NWYVDGVEVHNAKTKPREEQFNSTYRVVSVLTVLHQDWLNGKEYKCKVSN
KGLPSSIEKTISKAKGQPREPQVYTLPPSQEQMTKNQVKLTCLVKGFYPS
DIAVEWESNGQPENNYKTTPPVLDSDGSFFLYSRLTVDKSRWQEGNVFSC
SVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 scFv (VL-linker-VH) hIgG4 Variant 2

(SEQ ID NO: 3437)

QSALTQPRSASGNPGQRVTISCSGSSSNMGRRPVNWYQQIPGTAPKLLIY
SQNQRPSGVPDRFSGSQSGTSASLTISGLQSEDEAEYFCAVWDDIGRVLQ
LGGGTQLAVLggggsggggsggggsEVQLVETGGGLIQPGRSLRLSCAAS
GFTFSSYAMHWVRQAPGKGLEWVAVISYAGEVKYYADSVKGRFTISRDNS
KNTLYLQMNSLRAEDTAVYYCARDPLPLHYYGMDVWGQGTTVTVSSESKY
GPPCPPCPAPPVA()GPSVFLFPPKPKDTLMISRTPEVTCVVVDVKQEDP
EVQFNWYVDGVEVHNAKTKPREEEFNSTYRVVSVLTVLHQDWLNGKEYKC
KVSNKGLPSSIEKTISKAKGQPREPQVYTLPPSQEEMTKNQVSLTCDVSG
FYPSDIAVEWESDGQPENNYKTTPPVLDSDGSFFLYSRLTVDKSRWEEGD
VFSCSVMHEALHNHYTQKSLSLSPG;
CPA.9.086 scFv (VH-linker-VL) hIgG4 Variant 2

(SEQ ID NO: 3447)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSggggsggggsggggsQSALTQPRSASGNP
GQRVTISCSGSSSNMGRRPVNWYQQIPGTAPKLLIYSQNQRPSGVPDRFS
GSQSGTSASLTISGLQSEDEAEYFCAVWDDIGRVLQLGGGTQLAVLESKY
GPPCPPCPAPPVA()GPSVFLFPPKPKDTLMISRTPEVTCVVVDVKQEDP
EVQFNWYVDGVEVHNAKTKPREEEFNSTYRVVSVLTVLHQDWLNGKEYKC
KVSNKGLPSSIEKTISKAKGQPREPQVYTLPPSQEEMTKNQVSLTCDVSG
FYPSDIAVEWESDGQPENNYKTTPPVLDSDGSFFLYSRLTVDKSRWEEGD
VFSCSVMHEALHNHYTQKSLSLSPG;
CPA.9.086 hIgG4 Variant 2

(SEQ ID NO: 3467)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSDTKVDKRVESKYGPPCPPCPAPPVA()GPSVFLFPPKPK
DTLMISRTPEVTCVVVDVKQEDPEVQFNWYVDGVEVHNAKTKPREEEFNS
TYRVVSVLTVLHQDWLNGKEYKCKVSNKGLPSSIEKTISKAKGQPREPQV
YTLPPSQEEMTKNQVSLTCDVSGFYPSDIAVEWESDGQPENNYKTTPPVL
DSDGSFFLYSRLTVDKSRWEEGDVFSCSVMHEALHNHYTQKSLSLSPG;
CHA.9.547.18 scFv (VL-linker-VH) hIgG4 Variant 2

(SEQ ID NO: 3537)

DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIKggggsggggsggggsEVQLVESGGGLVQPGGSLRLSCAASGFT
FSSYIMSWVRQAPGKGLEWVATISGGSKGQYYADSVKGRFTISRDNSKNT
LYLQMNSLRAEDTAVYYCAKWLLSYYAMDYWGQGTLVTVSSESKYGPPCP
PCPAPPVA()GPSVFLFPPKPKDTLMISRTPEVTCVVVDVKQEDPEVQFN
WYVDGVEVHNAKTKPREEEFNSTYRVVSVLTVLHQDWLNGKEYKCKVSNK
GLPSSIEKTISKAKGQPREPQVYTLPPSQEEMTKNQVSLTCDVSGFYPSD
IAVEWESDGQPENNYKTTPPVLDSDGSFFLYSRLTVDKSRWEEGDVFSCS
VMHEALHNHYTQKSLSLSPG;
CHA.9.547.18 scFv (VH-linker-VL) hIgG4 Variant 2

(SEQ ID NO: 3547)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSSggggsggggsggggsDIQMTQSPSSLSASVG
DRITITCRASQSMAIWLSWYQQKPGKAPKLLIYKASKSHTGVPSRFSGSG
SGTDFTLTISSLQPEDFATYYCQQGQSYPYTFGQGTKLEIKESKYGPPCP
PCPAPPVA()GPSVFLFPPKPKDTLMISRTPEVTCVVVDVKQEDPEVQFN
WYVDGVEVHNAKTKPREEEFNSTYRVVSVLTVLHQDWLNGKEYKCKVSNK
GLPSSIEKTISKAKGQPREPQVYTLPPSQEEMTKNQVSLTCDVSGFYPSD
IAVEWESDGQPENNYKTTPPVLDSDGSFFLYSRLTVDKSRWEEGDVFSCS
VMHEALHNHYTQKSLSLSPG;
and
CHA.9.547.18 hIgG4 Variant 2

(SEQ ID NO: 3567)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSDTKVDKRVESKYGPPCPPCPAPPVA()GPSVFLFPPKPKDT
LMISRTPEVTCVVVDVKQEDPEVQFNWYVDGVEVHNAKTKPREEEFNSTY
RVVSVLTVLHQDWLNGKEYKCKVSNKGLPSSIEKTISKAKGQPREPQVYT
LPPSQEEMTKNQVSLTCDVSGFYPSDIAVEWESDGQPENNYKTTPPVLDS
DGSFFLYSRLTVDKSRWEEGDVFSCSVMHEALHNHYTQKSLSLSPG.

In some embodiments, the anti-PVRIG portion of the anti-PVRIG/anti-TIGIT bispecific antibody comprises the following anti-PVRIG sequence

CHA.7.518.4 scFv (VL-linker-VH) hIgG4 Variant 2

(SEQ ID NO: 3337)

DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIKggggsggggsggggsQVQLVQSGAEVKKPGASVKVSCKASGYT
FTDYNINWVRQAPGQGLEWMGYIYPYIGGSGYAQKFQGRVTMTRDTSTST
VYMELSSLRSEDTAVYYCAREDKTARNAMDYWGQGTLVTVSSESKYGPPC
PPCPAPPVA()GPSVFLFPPKPKDTLMISRTPEVTCVVVDVKQEDPEVQF
NWYVDGVEVHNAKTKPREEEFNSTYRVVSVLTVLHQDWLNGKEYKCKVSN
KGLPSSIEKTISKAKGQPREPQVYTLPPSQEEMTKNQVSLTCDVSGFYPS
DIAVEWESDGQPENNYKTTPPVLDSDGSFFLYSRLTVDKSRWEEGDVFSC
SVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 scFv (VL-linker-VH) hIgG4 Variant 1
(SEQ ID NO: 3417)
QSALTQPRSASGNPGQRVTISCSGSSSNMGRRPVNWYQQIPGTAPKLLIYSQNQRPSGVPDRFSGSQSGTSA
SLTISGLQSEDEAEYFCAVWDDIGRVLQLGGGTQLAVLggggsggggsggggsEVQLVETGGGLIQPGRSLR
LSCAASGFTFSSYAMHWVRQAPGKGLEWVAVISYAGEVKYYADSVKGRFTISRDNSKNTLYLQMNSLRAEDT
AVYYCARDPLPLHYYGMDVWGQGTTVTVSSESKYGPPCPPCPAPPVA()GPSVFLFPPKPKDTLMISRTPEV
TCVVVDVKQEDPEVQFNWYVDGVEVHNAKTKPREEQFNSTYRVVSVLTVLHQDWLNGKEYKCKVSNKGLPSS
IEKTISKAKGQPREPQVYTLPPSQEQMTKNQVKLTCLVKGFYPSDIAVEWESNGQPENNYKTTPPVLDSDGS
FFLYSRLTVDKSRWQEGNVFSCSVMHEALHNHYTQKSLSLSPG;
CPA.9.086 scFv (VH-linker-VL) hIgG4 Variant 1
(SEQ ID NO: 3427)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSggggsggggsggggsQSALTQPR
SASGNPGQRVTISCSGSSSNMGRRPVNWYQQIPGTAPKLLIYSQNQRPSGVPDRFSGSQSGTSASLTISGLQ
SEDEAEYFCAVWDDIGRVLQLGGGTQLAVLESKYGPPCPPCPAPPVA()GPSVFLFPPKPKDTLMISRTPEV
TCVVVDVKQEDPEVQFNWYVDGVEVHNAKTKPREEQFNSTYRVVSVLTVLHQDWLNGKEYKCKVSNKGLPSS
IEKTISKAKGQPREPQVYTLPPSQEQMTKNQVKLTCLVKGFYPSDIAVEWESNGQPENNYKTTPPVLDSDGS
FFLYSRLTVDKSRWQEGNVFSCSVMHEALHNHYTQKSLSLSPG;
CPA.9.086 hIgG4 Variant 1
(SEQ ID NO: 3457)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASTKGPSVFPLAPCSRSTSESTA
ALGCLVKDYFPEPVTVSWNSGALTSGVHTFPAVLQSSGLYSLSSVVTVPSSSLGTKTYTCNVDHKPSDTKVD
KRVESKYGPPCPPCPAPPVA()GPSVFLFPPKPKDTLMISRTPEVTCVVVDVKQEDPEVQFNWYVDGVEVHN
AKTKPREEQFNSTYRVVSVLTVLHQDWLNGKEYKCKVSNKGLPSSIEKTISKAKGQPREPQVYTLPPSQEQM
TKNQVKLTCLVKGFYPSDIAVEWESNGQPENNYKTTPPVLDSDGSFFLYSRLTVDKSRWQEGNVFSCSVMHE
ALHNHYTQKSLSLSPG;
CHA.9.547.18 scFv (VL-linker-VH) hIgG4 Variant 1
(SEQ ID NO: 3517)
DIQMTQSPSSLSASVGDRITITCRASQSMAIWLSWYQQKPGKAPKLLIYKASKSHTGVPSRFSGSGSGTDFT
LTISSLQPEDFATYYCQQGQSYPYTFGQGTKLEIKggggsggggsggggsEVQLVESGGGLVQPGGSLRLSC
AASGFTFSSYIMSWVRQAPGKGLEWVATISGGSKGQYYADSVKGRFTISRDNSKNTLYLQMNSLRAEDTAVY
YCAKWLLSYYAMDYWGQGTLVTVSSESKYGPPCPPCPAPPVA()GPSVFLFPPKPKDTLMISRTPEVTCVVV
DVKQEDPEVQFNWYVDGVEVHNAKTKPREEQFNSTYRVVSVLTVLHQDWLNGKEYKCKVSNKGLPSSIEKTI
SKAKGQPREPQVYTLPPSQEQMTKNQVKLTCLVKGFYPSDIAVEWESNGQPENNYKTTPPVLDSDGSFFLYS
RLTVDKSRWQEGNVFSCSVMHEALHNHYTQKSLSLSPG;
CHA.9.547.18 scFv (VH-linker-VL) hIgG4 Variant 1
(SEQ ID NO: 3527)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSggggsggggsggggsDIQMTQSPSS
LSASVGDRITITCRASQSMAIWLSWYQQKPGKAPKLLIYKASKSHTGVPSRFSGSGSGTDFTLTISSLQPED
FATYYCQQGQSYPYTFGQGTKLEIKESKYGPPCPPCPAPPVA()GPSVFLFPPKPKDTLMISRTPEVTCVVV
DVKQEDPEVQFNWYVDGVEVHNAKTKPREEQFNSTYRVVSVLTVLHQDWLNGKEYKCKVSNKGLPSSIEKTI
SKAKGQPREPQVYTLPPSQEQMTKNQVKLTCLVKGFYPSDIAVEWESNGQPENNYKTTPPVLDSDGSFFLYS
RLTVDKSRWQEGNVFSCSVMHEALHNHYTQKSLSLSPG;
and
CHA.9.547.18 hIgG4 Variant 1
(SEQ ID NO: 3557)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSNTKVDKR
VESKYGPPCPPCPAPPVA()GPSVFLFPPKPKDTLMISRTPEVTCVVVDVKQEDPEVQFNWYVDGVEVHNAK
TKPREEQFNSTYRVVSVLTVLHQDWLNGKEYKCKVSNKGLPSSIEKTISKAKGQPREPQVYTLPPSQEQMTK
NQVKLTCLVKGFYPSDIAVEWESNGQPENNYKTTPPVLDSDGSFFLYSRLTVDKSRWQEGNVFSCSVMHEAL
HNHYTQKSLSLSPG.

In some embodiments, the anti-PVRIG portion of the anti-PVRIG/anti-TIGIT bispecific antibody comprises the following anti-PVRIG sequence

CHA.7.518.4 scFv (VH-linker-VL) hIgG4 Variant 2

(SEQ ID NO: 3347)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSggggsggggsggggsDIQMTQSPSSLSASV
GDRVTITCRVSENIYDVLAWYQQKPGKAPKLLIYEATNLAEGVPSRFSGS
GSGTDFTLTISSLQPEDFATYYCQHFWGTPYTFGQGTKLEIKESKYGPPC
PPCPAPPVA()GPSVFLFPPKPKDTLMISRTPEVTCVVVDVKQEDPEVQF
NWYVDGVEVHNAKTKPREEEFNSTYRVVSVLTVLHQDWLNGKEYKCKVSN
KGLPSSIEKTISKAKGQPREPQVYTLPPSQEEMTKNQVSLTCDVSGFYPS
DIAVEWESDGQPENNYKTTPPVLDSDGSFFLYSRLTVDKSRWEEGDVFSC
SVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 scFv (VL-linker-VH) hIgG4 Variant 1
(SEQ ID NO: 3417)
QSALTQPRSASGNPGQRVTISCSGSSSNMGRRPVNWYQQIPGTAPKLLIYSQNQRPSGVPDRFSGSQSGTSA
SLTISGLQSEDEAEYFCAVWDDIGRVLQLGGGTQLAVLggggsggggsggggsEVQLVETGGGLIQPGRSLR
LSCAASGFTFSSYAMHWVRQAPGKGLEWVAVISYAGEVKYYADSVKGRFTISRDNSKNTLYLQMNSLRAEDT
AVYYCARDPLPLHYYGMDVWGQGTTVTVSSESKYGPPCPPCPAPPVA()GPSVFLFPPKPKDTLMISRTPEV
TCVVVDVKQEDPEVQFNWYVDGVEVHNAKTKPREEQFNSTYRVVSVLTVLHQDWLNGKEYKCKVSNKGLPSS
IEKTISKAKGQPREPQVYTLPPSQEQMTKNQVKLTCLVKGFYPSDIAVEWESNGQPENNYKTTPPVLDSDGS
FFLYSRLTVDKSRWQEGNVFSCSVMHEALHNHYTQKSLSLSPG;
CPA.9.086 scFv (VH-linker-VL) hIgG4 Variant 1
(SEQ ID NO: 3427)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSggggsggggsggggsQSALTQPR
SASGNPGQRVTISCSGSSSNMGRRPVNWYQQIPGTAPKLLIYSQNQRPSGVPDRFSGSQSGTSASLTISGLQ
SEDEAEYFCAVWDDIGRVLQLGGGTQLAVLESKYGPPCPPCPAPPVA()GPSVFLFPPKPKDTLMISRTPEV
TCVVVDVKQEDPEVQFNWYVDGVEVHNAKTKPREEQFNSTYRVVSVLTVLHQDWLNGKEYKCKVSNKGLPSS
IEKTISKAKGQPREPQVYTLPPSQEQMTKNQVKLTCLVKGFYPSDIAVEWESNGQPENNYKTTPPVLDSDGS
FFLYSRLTVDKSRWQEGNVFSCSVMHEALHNHYTQKSLSLSPG;
CPA.9.086 hIgG4 Variant 1
(SEQ ID NO: 3457)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASTKGPSVFPLAPCSRSTSESTA
ALGCLVKDYFPEPVTVSWNSGALTSGVHTFPAVLQSSGLYSLSSVVTVPSSSLGTKTYTCNVDHKPSDTKVD
KRVESKYGPPCPPCPAPPVA()GPSVFLFPPKPKDTLMISRTPEVTCVVVDVKQEDPEVQFNWYVDGVEVHN
AKTKPREEQFNSTYRVVSVLTVLHQDWLNGKEYKCKVSNKGLPSSIEKTISKAKGQPREPQVYTLPPSQEQM
TKNQVKLTCLVKGFYPSDIAVEWESNGQPENNYKTTPPVLDSDGSFFLYSRLTVDKSRWQEGNVFSCSVMHE
ALHNHYTQKSLSLSPG;
CHA.9.547.18 scFv (VL-linker-VH) hIgG4 Variant 1
(SEQ ID NO: 3517)
DIQMTQSPSSLSASVGDRITITCRASQSMAIWLSWYQQKPGKAPKLLIYKASKSHTGVPSRFSGSGSGTDFT
LTISSLQPEDFATYYCQQGQSYPYTFGQGTKLEIKggggsggggsggggsEVQLVESGGGLVQPGGSLRLSC
AASGFTFSSYIMSWVRQAPGKGLEWVATISGGSKGQYYADSVKGRFTISRDNSKNTLYLQMNSLRAEDTAVY
YCAKWLLSYYAMDYWGQGTLVTVSSESKYGPPCPPCPAPPVA()GPSVFLFPPKPKDTLMISRTPEVTCVVV
DVKQEDPEVQFNWYVDGVEVHNAKTKPREEQFNSTYRVVSVLTVLHQDWLNGKEYKCKVSNKGLPSSIEKTI
SKAKGQPREPQVYTLPPSQEQMTKNQVKLTCLVKGFYPSDIAVEWESNGQPENNYKTTPPVLDSDGSFFLYS
RLTVDKSRWQEGNVFSCSVMHEALHNHYTQKSLSLSPG;
CHA.9.547.18 scFv (VH-linker-VL) hIgG4 Variant 1
(SEQ ID NO: 3527)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSggggsggggsggggsDIQMTQSPSS
LSASVGDRITITCRASQSMAIWLSWYQQKPGKAPKLLIYKASKSHTGVPSRFSGSGSGTDFTLTISSLQPED
FATYYCQQGQSYPYTFGQGTKLEIKESKYGPPCPPCPAPPVA()GPSVFLFPPKPKDTLMISRTPEVTCVVV
DVKQEDPEVQFNWYVDGVEVHNAKTKPREEQFNSTYRVVSVLTVLHQDWLNGKEYKCKVSNKGLPSSIEKTI
SKAKGQPREPQVYTLPPSQEQMTKNQVKLTCLVKGFYPSDIAVEWESNGQPENNYKTTPPVLDSDGSFFLYS
RLTVDKSRWQEGNVFSCSVMHEALHNHYTQKSLSLSPG;
and
CHA.9.547.18 hIgG4 Variant 1
(SEQ ID NO: 3557)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSNTKVDKR
VESKYGPPCPPCPAPPVA()GPSVFLFPPKPKDTLMISRTPEVTCVVVDVKQEDPEVQFNWYVDGVEVHNAK
TKPREEQFNSTYRVVSVLTVLHQDWLNGKEYKCKVSNKGLPSSIEKTISKAKGQPREPQVYTLPPSQEQMTK
NQVKLTCLVKGFYPSDIAVEWESNGQPENNYKTTPPVLDSDGSFFLYSRLTVDKSRWQEGNVFSCSVMHEAL
HNHYTQKSLSLSPG.

In some embodiments, the anti-PVRIG portion of the anti-PVRIG/anti-TIGIT bispecific antibody comprises the following anti-PVRIG sequence

CHA.7.518.4 hIgG4 Variant 1

(SEQ ID NO: 3357)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPPVA()GPSVFLFPPKPKD
TLMISRTPEVTCVVVDVKQEDPEVQFNWYVDGVEVHNAKTKPREEQFNST
YRVVSVLTVLHQDWLNGKEYKCKVSNKGLPSSIEKTISKAKGQPREPQVY
TLPPSQEQMTKNQVKLTCLVKGFYPSDIAVEWESNGQPENNYKTTPPVLD
SDGSFFLYSRLTVDKSRWQEGNVFSCSVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 scFv (VL-linker-VH) hIgG4 Variant 2
(SEQ ID NO: 3437)
QSALTQPRSASGNPGQRVTISCSGSSSNMGRRPVNWYQQIPGTAPKLLIYSQNQRPSGVPDRFSGSQSGTSA
SLTISGLQSEDEAEYFCAVWDDIGRVLQLGGGTQLAVLggggsggggsggggsEVQLVETGGGLIQPGRSLR
LSCAASGFTFSSYAMHWVRQAPGKGLEWVAVISYAGEVKYYADSVKGRFTISRDNSKNTLYLQMNSLRAEDT
AVYYCARDPLPLHYYGMDVWGQGTTVTVSSESKYGPPCPPCPAPPVA()GPSVFLFPPKPKDTLMISRTPEV
TCVVVDVKQEDPEVQFNWYVDGVEVHNAKTKPREEEFNSTYRVVSVLTVLHQDWLNGKEYKCKVSNKGLPSS
IEKTISKAKGQPREPQVYTLPPSQEEMTKNQVSLTCDVSGFYPSDIAVEWESDGQPENNYKTTPPVLDSDGS
FFLYSRLTVDKSRWEEGDVFSCSVMHEALHNHYTQKSLSLSPG;
CPA.9.086 scFv (VH-linker-VL) hIgG4 Variant 2
(SEQ ID NO: 3447)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSggggsggggsggggsQSALTQPR
SASGNPGQRVTISCSGSSSNMGRRPVNWYQQIPGTAPKLLIYSQNQRPSGVPDRFSGSQSGTSASLTISGLQ
SEDEAEYFCAVWDDIGRVLQLGGGTQLAVLESKYGPPCPPCPAPPVA()GPSVFLFPPKPKDTLMISRTPEV
TCVVVDVKQEDPEVQFNWYVDGVEVHNAKTKPREEEFNSTYRVVSVLTVLHQDWLNGKEYKCKVSNKGLPSS
IEKTISKAKGQPREPQVYTLPPSQEEMTKNQVSLTCDVSGFYPSDIAVEWESDGQPENNYKTTPPVLDSDGS
FFLYSRLTVDKSRWEEGDVFSCSVMHEALHNHYTQKSLSLSPG;
CPA.9.086 hIgG4 Variant 2
(SEQ ID NO: 3467)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASTKGPSVFPLAPCSRSTSESTA
ALGCLVKDYFPEPVTVSWNSGALTSGVHTFPAVLQSSGLYSLSSVVTVPSSSLGTKTYTCNVDHKPSDTKVD
KRVESKYGPPCPPCPAPPVA()GPSVFLFPPKPKDTLMISRTPEVTCVVVDVKQEDPEVQFNWYVDGVEVHN
AKTKPREEEFNSTYRVVSVLTVLHQDWLNGKEYKCKVSNKGLPSSIEKTISKAKGQPREPQVYTLPPSQEEM
TKNQVSLTCDVSGFYPSDIAVEWESDGQPENNYKTTPPVLDSDGSFFLYSRLTVDKSRWEEGDVFSCSVMHE
ALHNHYTQKSLSLSPG;
CHA.9.547.18 scFv (VL-linker-VH) hIgG4 Variant 2
(SEQ ID NO: 3537)
DIQMTQSPSSLSASVGDRITITCRASQSMAIWLSWYQQKPGKAPKLLIYKASKSHTGVPSRFSGSGSGTDFT
LTISSLQPEDFATYYCQQGQSYPYTFGQGTKLEIKggggsggggsggggsEVQLVESGGGLVQPGGSLRLSC
AASGFTFSSYIMSWVRQAPGKGLEWVATISGGSKGQYYADSVKGRFTISRDNSKNTLYLQMNSLRAEDTAVY
YCAKWLLSYYAMDYWGQGTLVTVSSESKYGPPCPPCPAPPVA()GPSVFLFPPKPKDTLMISRTPEVTCVVV
DVKQEDPEVQFNWYVDGVEVHNAKTKPREEEFNSTYRVVSVLTVLHQDWLNGKEYKCKVSNKGLPSSIEKTI
SKAKGQPREPQVYTLPPSQEEMTKNQVSLTCDVSGFYPSDIAVEWESDGQPENNYKTTPPVLDSDGSFFLYS
RLTVDKSRWEEGDVFSCSVMHEALHNHYTQKSLSLSPG;
CHA.9.547.18 scFv (VH-linker-VL) hIgG4 Variant 2
(SEQ ID NO: 3547)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSggggsggggsggggsDIQMTQSPSS
LSASVGDRITITCRASQSMAIWLSWYQQKPGKAPKLLIYKASKSHTGVPSRFSGSGSGTDFTLTISSLQPED
FATYYCQQGQSYPYTFGQGTKLEIKESKYGPPCPPCPAPPVA()GPSVFLFPPKPKDTLMISRTPEVTCVVV
DVKQEDPEVQFNWYVDGVEVHNAKTKPREEEFNSTYRVVSVLTVLHQDWLNGKEYKCKVSNKGLPSSIEKTI
SKAKGQPREPQVYTLPPSQEEMTKNQVSLTCDVSGFYPSDIAVEWESDGQPENNYKTTPPVLDSDGSFFLYS
RLTVDKSRWEEGDVFSCSVMHEALHNHYTQKSLSLSPG;
and
CHA.9.547.18 hIgG4 Variant 2
(SEQ ID NO: 3567)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSDTKVDKR
VESKYGPPCPPCPAPPVA()GPSVFLFPPKPKDTLMISRTPEVTCVVVDVKQEDPEVQFNWYVDGVEVHNAK
TKPREEEFNSTYRVVSVLTVLHQDWLNGKEYKCKVSNKGLPSSIEKTISKAKGQPREPQVYTLPPSQEEMTK
NQVSLTCDVSGFYPSDIAVEWESDGQPENNYKTTPPVLDSDGSFFLYSRLTVDKSRWEEGDVFSCSVMHEAL
HNHYTQKSLSLSPG.

In some embodiments, the anti-PVRIG portion of the anti-PVRIG/anti-TIGIT bispecific antibody comprises the following anti-PVRIG sequence

CHA.7.518.4 hIgG4 Variant 2

(SEQ ID NO: 3367)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSDTKVDKRVESKYGPPCPPCPAPPVA()GPSVFLFPPKPKD
TLMISRTPEVTCVVVDVKQEDPEVQFNWYVDGVEVHNAKTKPREEEFNST
YRVVSVLTVLHQDWLNGKEYKCKVSNKGLPSSIEKTISKAKGQPREPQVY
TLPPSQEEMTKNQVSLTCDVSGFYPSDIAVEWESDGQPENNYKTTPPVLD
SDGSFFLYSRLTVDKSRWEEGDVFSCSVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 scFv (VL-linker-VH) hIgG4 Variant 1
(SEQ ID NO: 3417)
QSALTQPRSASGNPGQRVTISCSGSSSNMGRRPVNWYQQIPGTAPKLLIYSQNQRPSGVPDRFSGSQSGTSA
SLTISGLQSEDEAEYFCAVWDDIGRVLQLGGGTQLAVLggggsggggsggggsEVQLVETGGGLIQPGRSLR
LSCAASGFTFSSYAMHWVRQAPGKGLEWVAVISYAGEVKYYADSVKGRFTISRDNSKNTLYLQMNSLRAEDT
AVYYCARDPLPLHYYGMDVWGQGTTVTVSSESKYGPPCPPCPAPPVA()GPSVFLFPPKPKDTLMISRTPEV
TCVVVDVKQEDPEVQFNWYVDGVEVHNAKTKPREEQFNSTYRVVSVLTVLHQDWLNGKEYKCKVSNKGLPSS
IEKTISKAKGQPREPQVYTLPPSQEQMTKNQVKLTCLVKGFYPSDIAVEWESNGQPENNYKTTPPVLDSDGS
FFLYSRLTVDKSRWQEGNVFSCSVMHEALHNHYTQKSLSLSPG;
CPA.9.086 scFv (VH-linker-VL) hIgG4 Variant 1
(SEQ ID NO: 3427)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSggggsggggsggggsQSALTQPR
SASGNPGQRVTISCSGSSSNMGRRPVNWYQQIPGTAPKLLIYSQNQRPSGVPDRFSGSQSGTSASLTISGLQ
SEDEAEYFCAVWDDIGRVLQLGGGTQLAVLESKYGPPCPPCPAPPVA()GPSVFLFPPKPKDTLMISRTPEV
TCVVVDVKQEDPEVQFNWYVDGVEVHNAKTKPREEQFNSTYRVVSVLTVLHQDWLNGKEYKCKVSNKGLPSS
IEKTISKAKGQPREPQVYTLPPSQEQMTKNQVKLTCLVKGFYPSDIAVEWESNGQPENNYKTTPPVLDSDGS
FFLYSRLTVDKSRWQEGNVFSCSVMHEALHNHYTQKSLSLSPG;
CPA.9.086 hIgG4 Variant 1
(SEQ ID NO: 3457)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASTKGPSVFPLAPCSRSTSESTA
ALGCLVKDYFPEPVTVSWNSGALTSGVHTFPAVLQSSGLYSLSSVVTVPSSSLGTKTYTCNVDHKPSDTKVD
KRVESKYGPPCPPCPAPPVA()GPSVFLFPPKPKDTLMISRTPEVTCVVVDVKQEDPEVQFNWYVDGVEVHN
AKTKPREEQFNSTYRVVSVLTVLHQDWLNGKEYKCKVSNKGLPSSIEKTISKAKGQPREPQVYTLPPSQEQM
TKNQVKLTCLVKGFYPSDIAVEWESNGQPENNYKTTPPVLDSDGSFFLYSRLTVDKSRWQEGNVFSCSVMHE
ALHNHYTQKSLSLSPG;
CHA.9.547.18 scFv (VL-linker-VH) hIgG4 Variant 1
(SEQ ID NO: 3517)
DIQMTQSPSSLSASVGDRITITCRASQSMAIWLSWYQQKPGKAPKLLIYKASKSHTGVPSRFSGSGSGTDFT
LTISSLQPEDFATYYCQQGQSYPYTFGQGTKLEIKggggsggggsggggsEVQLVESGGGLVQPGGSLRLSC
AASGFTFSSYIMSWVRQAPGKGLEWVATISGGSKGQYYADSVKGRFTISRDNSKNTLYLQMNSLRAEDTAVY
YCAKWLLSYYAMDYWGQGTLVTVSSESKYGPPCPPCPAPPVA()GPSVFLFPPKPKDTLMISRTPEVTCVVV
DVKQEDPEVQFNWYVDGVEVHNAKTKPREEQFNSTYRVVSVLTVLHQDWLNGKEYKCKVSNKGLPSSIEKTI
SKAKGQPREPQVYTLPPSQEQMTKNQVKLTCLVKGFYPSDIAVEWESNGQPENNYKTTPPVLDSDGSFFLYS
RLTVDKSRWQEGNVFSCSVMHEALHNHYTQKSLSLSPG;
CHA.9.547.18 scFv (VH-linker-VL) hIgG4 Variant 1
(SEQ ID NO: 3527)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSggggsggggsggggsDIQMTQSPSS
LSASVGDRITITCRASQSMAIWLSWYQQKPGKAPKLLIYKASKSHTGVPSRFSGSGSGTDFTLTISSLQPED
FATYYCQQGQSYPYTFGQGTKLEIKESKYGPPCPPCPAPPVA()GPSVFLFPPKPKDTLMISRTPEVTCVVV
DVKQEDPEVQFNWYVDGVEVHNAKTKPREEQFNSTYRVVSVLTVLHQDWLNGKEYKCKVSNKGLPSSIEKTI
SKAKGQPREPQVYTLPPSQEQMTKNQVKLTCLVKGFYPSDIAVEWESNGQPENNYKTTPPVLDSDGSFFLYS
RLTVDKSRWQEGNVFSCSVMHEALHNHYTQKSLSLSPG;
and
CHA.9.547.18 hIgG4 Variant 1
(SEQ ID NO: 3557)

EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSNTKVDKR
VESKYGPPCPPCPAPPVA()GPSVFLFPPKPKDTLMISRTPEVTCVVVDVKQEDPEVQFNWYVDGVEVHNAK
TKPREEQFNSTYRVVSVLTVLHQDWLNGKEYKCKVSNKGLPSSIEKTISKAKGQPREPQVYTLPPSQEQMTK
NQVKLTCLVKGFYPSDIAVEWESNGQPENNYKTTPPVLDSDGSFFLYSRLTVDKSRWQEGNVFSCSVMHEAL
HNHYTQKSLSLSPG.

In some embodiments, the anti-PVRIG portion of the anti-PVRIG/anti-TIGIT bispecific antibody comprises the following anti-PVRIG sequence

CHA.7.518.4 VH Clambda-VL-CH1 Crossmab hIgG4
Fc Y349C E356D L358L T366S L368A Y407V Hole

(SEQ ID NO: 3661)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASVAAPSVFIFPPSDEQLKSGTASVVCLLN
NFYPREAKVQWKVDNALQSGNSQESVTEQDSKDSTYSLSSTLTLSKADYE
KHKVYACEVTHQGLSSPVTKSFNRGECESKYGPPCPPCPAPEFLGGPSVF
LFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKG
QPREPQVCTLPPSQDELTKNQVSLSCAVKGFYPSDIAVEWESNGQPENNY
KTTPPVLDSDGSFFLVSRLTVDKSRWQEGNVFSCSVMHEALHNHYTQKSL
SLSPG;

and the anti-TIGIT portion of the anti-PVRIG/anti-TIGIT bispecific antibody comprises an anti-TIGIT sequence selected from the group consisting of the following sequences:

CPA.9.086 VH Clambda-VL-CH1 Crossmab hIgG4 Fc Y354C E356D M348L T366W
Knob
(SEQ ID NO: 3487)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASGQPKAAPSVTLFPPSSEELQA
NKATLVCLISDFYPGAVTVAWKADSSPVKAGVETTTPSKQSNNKYAASSYLSLTPEQWKSHRSYSCQVTHEG
STVEKTVAPTECSESKYGPPCPPCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREPQVY
TLPPCQDELTKNQVSLWCLVKGFYPSDIAVEWESNGQPENNYKTTPPVLDSDGSFFLYSRLTVDKSRWQEGN
VFSCSVMHEALHNHYTQKSLSLSPG;
CPA.9.086 VH hIgG4 Fc Y354C E356D M348L T366W Knob
(SEQ ID NO: 3507)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASASTKGPSVFPLAPCSRSTSES
TAALGCLVKDYFPEPVTVSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAKGQPREPQVYTLPPCQDE
LTKNQVSLWCLVKGFYPSDIAVEWESNGQPENNYKTTPPVLDSDGSFFLYSRLTVDKSRWQEGNVFSCSVMH
EALHNHYTQKSLSLSPG;
CHA.9.547.18 VH Clambda-VL-CH1 Crossmab hIgG4 Fc Y354C E356D M348L
T366W Knob
(SEQ ID NO: 3662)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASVAAPSVFIFPPSDEQLKSGTASV
VCLLNNFYPREAKVQWKVDNALQSGNSQESVTEQDSKDSTYSLSSTLTLSKADYEKHKVYACEVTHQGLSSP
VTKSFNRGECESKYGPPCPPCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISKAKGQPREPQVYTLP
PCQDELTKNQVSLWCLVKGFYPSDIAVEWESNGQPENNYKTTPPVLDSDGSFFLYSRLTVDKSRWQEGNVFS
CSVMHEALHNHYTQKSLSLSPG;
and
CHA.9.547.18 VH hIgG4 Fc Y354C E356D M348L T366W Knob
(SEQ ID NO: 3607)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASASTKGPSVFPLAPCSRSTSESTA
ALGCLVKDYFPEPVTVSWNSGALTSGVHTFPAVLQSSGLYSLSSVVTVPSSSLGTKTYTCNVDHKPSNTKVD
KRVESKYGPPCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVYTLPPCQDELT
KNQVSLWCLVKGFYPSDIAVEWESNGQPENNYKTTPPVLDSDGSFFLYSRLTVDKSRWQEGNVFSCSVMHEA
LHNHYTQKSLSLSPG.

In some embodiments, the anti-PVRIG portion of the anti-PVRIG/anti-TIGIT bispecific antibody comprises the following anti-PVRIG sequence

CHA.7.518.4 VH Clambda-VL-CH1 Crossmab hIgG4
Fc Y354C E356D M348L T366W Knob

(SEQ ID NO: 3663)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASVAAPSVFIFPPSDEQLKSGTASVVCLLN
NFYPREAKVQWKVDNALQSGNSQESVTEQDSKDSTYSLSSTLTLSKADYE
KHKVYACEVTHQGLSSPVTKSFNRGECESKYGPPCPPCPAPEFLGGPSVF
LFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKG
QPREPQVYTLPPCQDELTKNQVSLWCLVKGFYPSDIAVEWESNGQPENNY
KTTPPVLDSDGSFFLYSRLTVDKSRWQEGNVFSCSVMHEALHNHYTQKSL
SLSPG;

and the anti-TIGIT portion of the anti-PVRIG/anti-TIGIT bispecific antibody comprises an anti-TIGIT sequence selected from the group consisting of the following sequences:

CPA.9.086 VH Clambda-VL-CH1 Crossmab hIgG4 Fc Y349C E356D L358L T366S
L368A Y407V Hole
(SEQ ID NO: 3477)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASGQPKAAPSVTLFPPSSEELQA
NKATLVCLISDFYPGAVTVAWKADSSPVKAGVETTTPSKQSNNKYAASSYLSLTPEQWKSHRSYSCQVTHEG
STVEKTVAPTECSESKYGPPCPPCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREPQVC
TLPPSQDELTKNQVSLSCAVKGFYPSDIAVEWESNGQPENNYKTTPPVLDSDGSFFLVSRLTVDKSRWQEGN
VFSCSVMHEALHNHYTQKSLSLSPG;
CPA.9.086 VH hIgG4 Fc Y349C E356D L358L T366S L368A Y407V Hole
(SEQ ID NO: 3497)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASASTKGPSVFPLAPCSRSTSES
TAALGCLVKDYFPEPVTVSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAKGQPREPQVCTLPPSQDE
LTKNQVSLSCAVKGFYPSDIAVEWESNGQPENNYKTTPPVLDSDGSFFLVSRLTVDKSRWQEGNVFSCSVMH
EALHNHYTQKSLSLSPG;
CHA.9.547.18 VH Clambda-VL-CH1 Crossmab hIgG4 Fc Y349C E356D L358L
T366S L368A Y407V Hole
(SEQ ID NO: 3664)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASVAAPSVFIFPPSDEQLKSGTASV
VCLLNNFYPREAKVQWKVDNALQSGNSQESVTEQDSKDSTYSLSSTLTLSKADYEKHKVYACEVTHQGLSSP
VTKSFNRGECESKYGPPCPPCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISKAKGQPREPQVCTLP
PSQDELTKNQVSLSCAVKGFYPSDIAVEWESNGQPENNYKTTPPVLDSDGSFFLVSRLTVDKSRWQEGNVFS
CSVMHEALHNHYTQKSLSLSPG;
and
CHA.9.547.18 VH hIgG4 Fc Y349C E356D L358L T366S L368A Y407V Hole
(SEQ ID NO: 3597)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASASTKGPSVFPLAPCSRSTSESTA
ALGCLVKDYFPEPVTVSWNSGALTSGVHTFPAVLQSSGLYSLSSVVTVPSSSLGTKTYTCNVDHKPSNTKVD
KRVESKYGPPCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVCTLPPSQDELT
KNQVSLSCAVKGFYPSDIAVEWESNGQPENNYKTTPPVLDSDGSFFLVSRLTVDKSRWQEGNVFSCSVMHEA
LHNHYTQKSLSLSPG.

In some embodiments, the anti-PVRIG portion of the anti-PVRIG/anti-TIGIT bispecific antibody comprises the following anti-PVRIG sequence

CHA.7.518.4 VH hIgG4 Fc Y349C E356D L358L T366S
L368A Y407V Hole
(SEQ ID NO: 3397)
QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASASTKGPSVFPLAPCSRSTSESTAALGCL
VKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPK
DTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQV
CTLPPSQDELTKNQVSLSCAVKGFYPSDIAVEWESNGQPENNYKTTPPVL
DSDGSFFLVSRLTVDKSRWQEGNVFSCSVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 VH Clambda-VL-CH1 Crossmab hIgG4 Fc Y354C E356D M348L T366W
Knob
(SEQ ID NO: 3487)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASGQPKAAPSVTLFPPSSEELQA
NKATLVCLISDFYPGAVTVAWKADSSPVKAGVETTTPSKQSNNKYAASSYLSLTPEQWKSHRSYSCQVTHEG
STVEKTVAPTECSESKYGPPCPPCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREPQVY
TLPPCQDELTKNQVSLWCLVKGFYPSDIAVEWESNGQPENNYKTTPPVLDSDGSFFLYSRLTVDKSRWQEGN
VFSCSVMHEALHNHYTQKSLSLSPG;
CPA.9.086 VH hIgG4 Fc Y354C E356D M348L T366W Knob
(SEQ ID NO: 3507)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASASTKGPSVFPLAPCSRSTSES
TAALGCLVKDYFPEPVTVSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAKGQPREPQVYTLPPCQDE
LTKNQVSLWCLVKGFYPSDIAVEWESNGQPENNYKTTPPVLDSDGSFFLYSRLTVDKSRWQEGNVFSCSVMH
EALHNHYTQKSLSLSPG;
CHA.9.547.18 VH Clambda-VL-CH1 Crossmab hIgG4 Fc Y354C E356D M348L
T366W Knob
(SEQ ID NO: 3665)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASVAAPSVFIFPPSDEQLKSGTASV
VCLLNNFYPREAKVQWKVDNALQSGNSQESVTEQDSKDSTYSLSSTLTLSKADYEKHKVYACEVTHQGLSSP
VTKSFNRGECESKYGPPCPPCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISKAKGQPREPQVYTLP
PCQDELTKNQVSLWCLVKGFYPSDIAVEWESNGQPENNYKTTPPVLDSDGSFFLYSRLTVDKSRWQEGNVFS
CSVMHEALHNHYTQKSLSLSPG;
and
CHA.9.547.18 VH hIgG4 Fc Y354C E356D M348L T366W Knob
(SEQ ID NO: 3607)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASASTKGPSVFPLAPCSRSTSESTA
ALGCLVKDYFPEPVTVSWNSGALTSGVHTFPAVLQSSGLYSLSSVVTVPSSSLGTKTYTCNVDHKPSNTKVD
KRVESKYGPPCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVYTLPPCQDELT
KNQVSLWCLVKGFYPSDIAVEWESNGQPENNYKTTPPVLDSDGSFFLYSRLTVDKSRWQEGNVFSCSVMHEA
LHNHYTQKSLSLSPG.

In some embodiments, the anti-PVRIG portion of the anti-PVRIG/anti-TIGIT bispecific antibody comprises the following anti-PVRIG sequence

CHA.7.518.4 VH hIgG4 Fc Y354C E356D M348L T366W
Knob

(SEQ ID NO: 3407)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASASTKGPSVFPLAPCSRSTSESTAALGCL
VKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPK
DTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQV
YTLPPCQDELTKNQVSLWCLVKGFYPSDIAVEWESNGQPENNYKTTPPVL
DSDGSFFLYSRLTVDKSRWQEGNVFSCSVMHEALHNHYTQKSLSLSPG;

and the anti-TIGIT portion of the anti-PVRIG/anti-TIGIT bispecific antibody comprises an anti-TIGIT sequence selected from the group consisting of the following sequences:

CPA.9.086 VH Clambda-VL-CH1 Crossmab hIgG4 Fc Y349C E356D L358L T366S
L368A Y407V Hole
(SEQ ID NO: 3477)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASGQPKAAPSVTLFPPSSEELQA
NKATLVCLISDFYPGAVTVAWKADSSPVKAGVETTTPSKQSNNKYAASSYLSLTPEQWKSHRSYSCQVTHEG
STVEKTVAPTECSESKYGPPCPPCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREPQVC
TLPPSQDELTKNQVSLSCAVKGFYPSDIAVEWESNGQPENNYKTTPPVLDSDGSFFLVSRLTVDKSRWQEGN
VFSCSVMHEALHNHYTQKSLSLSPG;
CPA.9.086 VH hIgG4 Fc Y349C E356D L358L T366S L368A Y407V Hole
(SEQ ID NO: 3497)
EVQLVETGGGLIQPGRSLRLSCAASGFTFSSYAMHWVRQAPGKGLEWVAVISYAGEVKYYADSVKGRFTISR
DNSKNTLYLQMNSLRAEDTAVYYCARDPLPLHYYGMDVWGQGTTVTVSSASASTKGPSVFPLAPCSRSTSES
TAALGCLVKDYFPEPVTVSWNSGALTSGVHTFPAVLQSSGLYSLSSVVTVPSSSLGTKTYTCNVDHKPSNTK
VDKRVESKYGPPCPPCPAPEFLGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAKGQPREPQVCTLPPSQDE
LTKNQVSLSCAVKGFYPSDIAVEWESNGQPENNYKTTPPVLDSDGSFFLVSRLTVDKSRWQEGNVFSCSVMH
EALHNHYTQKSLSLSPG;
CHA.9.547.18 VH Clambda-VL-CH1 Crossmab hIgG4 Fc Y349C E356D L358L
T366S L368A Y407V Hole
(SEQ ID NO: 3666)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASVAAPSVFIFPPSDEQLKSGTASV
VCLLNNFYPREAKVQWKVDNALQSGNSQESVTEQDSKDSTYSLSSTLTLSKADYEKHKVYACEVTHQGLSSP
VTKSFNRGECESKYGPPCPPCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISKAKGQPREPQVCTLP
PSQDELTKNQVSLSCAVKGFYPSDIAVEWESNGQPENNYKTTPPVLDSDGSFFLVSRLTVDKSRWQEGNVFS
CSVMHEALHNHYTQKSLSLSPG;
and
CHA.9.547.18 VH hIgG4 Fc Y349C E356D L358L T366S L368A Y407V Hole
(SEQ ID NO: 3597)
EVQLVESGGGLVQPGGSLRLSCAASGFTFSSYIMSWVRQAPGKGLEWVATISGGSKGQYYADSVKGRFTISR
DNSKNTLYLQMNSLRAEDTAVYYCAKWLLSYYAMDYWGQGTLVTVSSASASTKGPSVFPLAPCSRSTSESTA
ALGCLVKDYFPEPVTVSWNSGALTSGVHTFPAVLQSSGLYSLSSVVTVPSSSLGTKTYTCNVDHKPSNTKVD
KRVESKYGPPCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVCTLPPSQDELT
KNQVSLSCAVKGFYPSDIAVEWESNGQPENNYKTTPPVLDSDGSFFLVSRLTVDKSRWQEGNVFSCSVMHEA
LHNHYTQKSLSLSPG.

Additionally included in the definition of anti-PVRIG/anti-TIGIT bispecific antibodies are antibodies that comprise TIGIT binding domains that share identity to the binding domains from the TIGIT antibodies enumerated herein. That is, in certain embodiments, an anti-PVRIG/anti-TIGIT bispecific antibody according to the invention comprises heavy and light chain variable regions comprising amino acid sequences that are identical to all or part of the binding domains from the anti-TIGIT amino acid sequences of preferred anti-TIGIT antibodies, respectively, wherein the antibodies retain the desired functional properties of the parent anti-TIGIT antibodies. The percent identity between the two sequences is a function of the number of identical positions shared by the sequences (i.e., % homology=# of identical positions/total # of positions X 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J Mol. Biol. 215:403-10. BLAST protein searches can be performed with the XBLAST program, score=50, wordlength=3 to obtain amino acid sequences homologous to the antibody molecules according to at least some embodiments of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In general, the percentage identity for comparison between TIGIT binding domains or antigen binding domains is at least 75%, at least 80%, at least 90%, with at least about 95%, 96%, 97%, 98% or 99% percent identity being preferred. The percentage identity may be along the whole amino acid sequence, for example the entire heavy or light chain or along a portion of the chains. For example, included within the definition of the anti-PVRIG/anti-TIGIT bispecific antibodies of the invention are those whose TIGIT binding portion or antigen binding domains shares identity along the entire variable region (for example, where the identity is 95% or 98% identical along the variable regions), or along the entire constant region, or along just the Fc domain. In particular, the invention provides anti-PVRIG/anti-TIGIT bispecific antibodies that have TIGIT binding portions or antigen binding domains with at least 75%, at least 80%, at least 90%, with at least about 95%, 96%, 97%, 98%, or 99% percent identity being preferred, with the CPA.9.086 antibody. In particular, the invention provides anti-PVRIG/anti-TIGIT bispecific antibodies that have TIGIT binding portions or antigen binding domains with at least 75%, at least 80%, at least 90%, with at least about 95%, 96%, 97%, 98%, or 99% percent identity being preferred, with the CHA.9.547.18 antibody.

In addition, also included are sequences that may have the identical CDRs but changes in the framework portions of the variable domain (or entire heavy or light chain). For example, TIGIT antibodies include those with CDRs identical to those shown in FIG. 23A-23D but whose identity along the variable region can be lower, for example 95 or 98% percent identical. In particular, the invention provides anti-PVRIG/anti-TIGIT bispecific antibodies that have TIGIT binding portions or antigen binding domains with identical CDRs to CPA.9.086 but with framework regions that are 95% or 98% identical to CPA.9.086. In particular, the invention provides anti-PVRIG/anti-TIGIT bispecific antibodies that have TIGIT binding portions or antigen binding domains with identical CDRs to CHA.9.547.18but with framework regions that are 95% or 98% identical to CHA.9.547.18.

In addition, also included are sequences that may have the identical CDRs but changes in the framework portions of the variable domain (or entire heavy or light chain). For example, TIGIT antibodies include those with CDRs identical to those shown in FIG. 24A-24EE but whose identity along the variable region can be lower, for example 95 or 98% percent identical.

In some embodiments, the TIGIT binding portion is from an anti-TIGIT antibody as provided in US2016/0176963A1, (incorporated herein by reference in its entirety). In some embodiments, the TIGIT binding portion is from an anti-TIGIT antibody as provided in US20170281764 (incorporated herein by reference in its entirety). In some embodiments, the TIGIT binding portion is from an anti-TIGIT antibody as provided in WO2015009856 (incorporated herein by reference in its entirety). In some embodiments, the TIGIT binding portion is from an anti-TIGIT antibody as provided in U.S. Pat. No. 9,713,641 (incorporated herein by reference in its entirety). In some embodiments, the TIGIT binding portion is from an anti-TIGIT antibody as provided in WO2016028656 (incorporated herein by reference in its entirety).

1. TIGIT Antibodies that Compete for Binding

The present invention provides not only the enumerated antibodies but additional antibodies that compete with the enumerated antibodies (the CPA numbers enumerated herein that specifically bind to TIGIT) to specifically bind to the TIGIT molecule. The TIGIT antibodies of the invention “bin” into different epitope bins. Among the 44 TIGIT antibodies in the epitope binning study, there are four communities, each having related pairwise blocking patterns, which separate into 12 total discrete bins outlined herein. There are twelve discrete bins outlined herein; 1) BM9-H4, CHA.9.525, CPA.9.081-H4, CHA.9.538, CHA.9.553, CPA.9.069-H4, CHA.9.543, CHA.9.556, CPA.9.077-H4 and CHA.9.561; 2) CHA.9.560 and CHA.9.528; 3) CHA.9.552, CHA.9.521, CHA.9.541, CHA.9.529, CHA.9.519, CHA.9.527 and CHA.9.549; 4) CPA.9.057-H4 and CHA.9.554; 5) CHA.9.546, CPA.9.012-H4, CHA.9.547, CPA.9.013-H4, CPA.9.018-H4, MBSA43-M1, Sino PVR-Fc(ligand), CHA.9.555, PVR-Fc M2A(ligand), BM29-H4, CPA.9.027-H4, CPA.9.049-H4 and CPA.9.053-H4; 6) CPA.9.064-H4; 7) BM26-H4; 8) CPA.9.059-H4; 9) CHA.9.535 and CPA.9.009-H4; 10) CHA.9.536, CHA.9.522 and CPA.9.015-H4; 11) CPA.9.011-H4 and BM8-H4 and 12) CPA.9.071-H4. As discussed in WO2018/033798, incorporated herein by reference in it's entirety.

Thus, the invention provides anti-PVRIG/anti-TIGIT bispecific antibodies that compete for binding with antibodies that are in discrete epitope bins 1 to 12. In a particular embodiment, the invention provides anti-PVRIG/anti-TIGIT bispecific antibodies that compete for binding with CPA.9.086 and are at least 95%, 96%, 97%, 98%, or 99% identical to CPA.9.086.

Additional antibodies anti-PVRIG/anti-TIGIT bispecific antibodies that compete with the enumerated antibodies are generated, as is known in the art and generally outlined below. Competitive binding studies can be done as is known in the art, generally using SPR/Biacore® binding assays, as well as ELISA and cell-based assays.

### VII. NUCLEIC ACIDS ENCODING ANTIBODIES

Nucleic acid compositions encoding the anti-PVRIG/anti-TIGIT bispecific antibodies of the invention are also provided, as well as expression vectors containing the nucleic acids and host cells transformed with the nucleic acid and/or expression vector compositions. As will be appreciated by those in the art, the protein sequences depicted herein can be encoded by any number of possible nucleic acid sequences, due to the degeneracy of the genetic code.

The nucleic acid compositions that encode the anti-PVRIG/anti-TIGIT bispecific antibodies will depend on the format of the antibody. For traditional, tetrameric antibodies containing two heavy chains and two light chains are encoded by two different nucleic acids, one encoding the heavy chain and one encoding the light chain. These can be put into a single expression vector or two expression vectors, as is known in the art, transformed into host cells, where they are expressed to form the antibodies of the invention. In some embodiments, for example when scFv constructs are used, a single nucleic acid encoding the variable heavy chain-linker-variable light chain is generally used, which can be inserted into an expression vector for transformation into host cells. The nucleic acids can be put into expression vectors that contain the appropriate transcriptional and translational control sequences, including, but not limited to, signal and secretion sequences, regulatory sequences, promoters, origins of replication, selection genes, etc.

Preferred mammalian host cells for expressing the recombinant anti-PVRIG/anti-TIGIT bispecific antibodies according to at least some embodiments of the invention include Chinese Hamster Ovary (CHO cells), PER.C6, HEK293 and others as is known in the art.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art.

To create a scFv gene, the V_{H}- and V_{L}-encoding DNA fragments are operatively linked to another fragment encoding a flexible linker, e.g., encoding the amino acid sequence (Gly₄-Ser)₃ and others discussed herein, such that the V_{H} and V_{L} sequences can be expressed as a contiguous single-chain protein, with the V_{L} and V_{H} regions joined by the flexible linker.

### VIII. FORMULATIONS

The therapeutic compositions used in the practice of the foregoing methods (and in particular bispecific antibodies comprising at least the CDRs from CHA.7.518.1, CHA.7.518.4, CPA.9.086, and/or CHA.9.547.18) can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16^{th} Edition, A. Osal., Ed., 1980). Acceptable carriers, excipients, or stabilizers are nontoxic to recipients at the dosages and concentrations employed and may include buffers.

In some embodiments, the anti-PVRIG/anti-TIGIT bispecific antibody formulated into a pharmaceutical composition comprises the following sequences:

CHA.7.518.1.H4(S241P) VH IgG1 CH1 IgG4 CH2—CH3
S354C, E356D, M358L, T366W

(SEQ ID NO: 3175)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPCQDELTKNQVSLWCLVKGFYPSDIAVEWESNGQPENNYKTTPPVLDS
DGSFFLYSRLTVDKSRWQEGNVFSCSVMHEALHNHYTQKSLSLSPGK;
CHA.7.518.1.H4(S241P) VL Ckappa

(SEQ ID NO: 3176)

DIQMTQSPSSLSASVGDRVTITCRVSENIYSNLAWYQQKPGKAPKLLIYE
ATNLAEGVPSRFSGSGSGTDFTLTISSLQPEDFATYYCQHFWGTPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC;
and
CPA.9.086 HC Clambda Crossmab IgG4 Y349C, E356D,
M358L, T366S, L368A, Y407V

(SEQ ID NO: 3177)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSASGQPKAAPSVTLFPPSSEELQANKATLV
CLISDFYPGAVTVAWKADSSPVKAGVETTTPSKQSNNKYAASSYLSLTPE
QWKSHRSYSCQVTHEGSTVEKTVAPTECSESKYGPPCPPCPAPEFLGGPS
VFLFPPKPKDTLMISRTPEVTCVVVDVSQEDPEVQFNWYVDGVEVHNAKT
KPREEQFNSTYRWSVLTVLHQDWLNGKEYKCKVSNKGLPSSIEKTISKAK
GQPREPQVCTLPPSQDELTKNQVSLSCAVKGFYPSDIAVEWESNGQPENN
YKTTPPVLDSDGSFFLVSRLTVDKSRWQEGNVFSCSVMHEALHNHYTQKS
LSLSPGK;
CPA.9.086 LC CH1

(SEQ ID NO: 3178)

QSALTQPRSASGNPGQRVTISCSGSSSNMGRRPVNWYQQIPGTAPKLLIY
SQNQRPSGVPDRFSGSQSGTSASLTISGLQSEDEAEYFCAVWDDIGRVLQ
LGGGTQLAVLSSASTKGPSVFPLAPSSKSTSGGTAALGCLVKDYFPEPVT
VSWNSGALTSGVHTFPAVLQSSGLYSLSSVVTVPSSSLGTQTYICNVNHK
PSNTKVDKKVEPKSC.

In some embodiments, the anti-PVRIG/anti-TIGIT bispecific antibody formulated into a pharmaceutical composition comprises the following sequences:

Antibody sequence for PVRIG (IMGT CDR
definition bold and underlined)
CHA.7.518.4 VH

(SEQ ID NO: 3179)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.4 VL

(SEQ ID NO: 3180)

DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIK;
and
Antibody sequence for TIGIT (IMGT CDR
definition bold and underlined)
CHA.9.547.18 VH

(SEQ ID NO: 1664)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSS;
CHA.9.547.18 VL

(SEQ ID NO: 1668)

DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIK.

In some embodiments, the anti-PVRIG antibody formulated into a pharmaceutical composition comprises the following sequences:

Antibody sequence for PVRIG (IMGT CDR
definition bold and underlined)
CHA.7.518.4 VH
(SEQ ID NO: 3179)
QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
and
CHA.7.518.4 VL
(SEQ ID NO: 3180)
DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIK.

In some embodiments, the anti-TIGIT antibody formulated into a pharmaceutical composition comprises the following sequences:

Antibody sequence for TIGIT (IMGT CDR
definition bold and underlined)
CHA.9.547.18 VH
(SEQ ID NO: 1664)
EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSS;
and
CHA.9.547.18 VL
(SEQ ID NO: 1668)
DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIK.

In a preferred embodiment, the pharmaceutical composition that comprises the anti-PVRIG/anti-TIGIT bispecific antibodies of the invention may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids and the like. “Pharmaceutically acceptable base addition salts” include those derived from inorganic bases and the like.

Administration of the pharmaceutical composition comprising anti-PVRIG/anti-TIGIT bispecific antibodies of the present invention, preferably in the form of a sterile aqueous solution, may be done in a variety of ways, including, but not limited to subcutaneously and intravenously.

The dosing amounts and frequencies of administration are, in a preferred embodiment, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

In order to treat a patient, a therapeutically effective dose of the Fc variant of the present invention may be administered. By “therapeutically effective dose” herein is meant a dose that produces the effects for which it is administered. The exact dose will depend on the purpose of the treatment, and will be ascertainable by one skilled in the art using known techniques.

### IX. METHODS FOR USING ANTIBODIES

The anti-PVRIG, anti-TIGIT, and/or anti-PVRIG/anti-TIGIT bispecific antibodies of the invention can be used in a number of diagnostic and therapeutic applications. In some cases, the decision of which anti-PVRIG/anti-TIGIT bispecific antibodies to administer to a patient is done using an evaluation of the expression levels (either gene expression levels or protein expression levels, with the latter being preferred) of sample tumor biopsies to determine whether the sample is overexpressing TIGIT and/or PVRIG, to determine what therapeutic antibody to administer.

A. Diagnostic Uses

Accordingly, the anti-PVRIG, anti-TIGIT, and/or anti-PVRIG/anti-TIGIT bispecific antibodies of the invention also find use in the in vitro or in vivo diagnosis, including imaging, of tumors that over-express either PVRIG and/or TIGIT, respectively. It should be noted, however, that as discussed herein, both TIGIT and PVRIG, as immuno-oncology target proteins, are not necessarily overexpressed on cancer cells, but rather within the immune infiltrates in the cancer (for example, in the tumor microenvironment, also referred to as the TME). Thus it is the mechanism of action, e.g., activation of immune cells such as T cells and NK cells, that results in cancer diagnosis. Accordingly, these antibodies can be used to diagnose cancer. Diagnosis using PVRIG antibodies is also outlined in WO 2016/134333, [0434 to 0459], hereby incorporated by reference.

Generally, diagnosis can be done in several ways. In one embodiment, a tissue from a patient, such as a biopsy sample, is contacted with a anti-PVRIG/anti-TIGIT bispecific antibodies antibody, generally labeled, such that the antibody binds to the endogenous TIGIT. The level of signal is compared to that of normal non-cancerous tissue either from the same patient or a reference sample, to determine the presence or absence of cancer. The biopsy sample can be from a solid tumor, a blood sample (for lymphomas and leukemias such as ALL, T cell lymphoma, etc).

In general, in this embodiment, the anti-PVRIG, anti-TIGIT, and/or anti-PVRIG/anti-TIGIT bispecific antibodies is labeled, for example with a fluorophore or other optical label, that is detected using a fluorometer or other optical detection system as is well known in the art. In an alternate embodiment, a secondary labeled antibody is contacted with the sample, for example using an anti-human IgG antibody from a different mammal (mouse, rat, rabbit, goat, etc.) to form a sandwich assay as is known in the art. Alternatively, the anti-PVRIG/anti-TIGIT bispecific antibodies could be directly labeled (e.g., with biotin) and detection can be done by a secondary Ab directed to the labeling agent in the art.

Once over-expression of TIGIT is seen, treatment can proceed with the administration of an anti-PVRIG, anti-TIGIT, and/or anti-PVRIG/anti-TIGIT bispecific antibody according to the invention as outlined herein.

In other embodiments, in vivo diagnosis is done. Generally, in this embodiment, the anti-TIGIT antibody (including antibody fragments) is injected into the patient and imaging is done. In this embodiment, for example, the anti-PVRIG, anti-TIGIT, and/or anti-PVRIG/anti-TIGIT bispecific antibody is generally labeled with an optical label or an MRI label, such as a gadolinium chelate, radioactive labeling of mAb (including fragments).

In some embodiments, the anti-PVRIG, anti-TIGIT, and/or anti-PVRIG/anti-TIGIT bispecific antibodies described herein are used for both diagnosis and treatment, or for diagnosis alone. When anti-PVRIG, anti-TIGIT, and/or anti-PVRIG/anti-TIGIT bispecific antibodies are used for both diagnosis and treatment, some embodiments rely on two different anti-PVRIG, anti-TIGIT, and/or anti-PVRIG/anti-TIGIT bispecific antibodies to two different epitopes (for example to two different eptitopes withing TIGIT or PVRIG, or based on their bispecific nature of binding to both PVRIG and TIGIT), such that the diagnostic anti-PVRIG, anti-TIGIT, and/or anti-PVRIG/anti-TIGIT bispecific antibody does not compete for binding with the therapeutic anti-PVRIG, anti-TIGIT, and/or anti-PVRIG/anti-TIGIT bispecific antibody, although in some cases the same antibody can be used for both. For example, this can be done using anti-PVRIG, anti-TIGIT, and/or anti-PVRIG/anti-TIGIT bispecific antibodies that are in different bins, e.g., that bind to different epitopes on TIGIT or different epitopes on PVRIG, such as outlined herein. Thus included in the invention are compositions comprising a diagnostic anti-PVRIG, anti-TIGIT, and/or anti-PVRIG/anti-TIGIT bispecific antibody and an anti-PVRIG, anti-TIGIT, and/or anti-PVRIG/anti-TIGIT bispecific antibody, and in some embodiments, the diagnostic anti-PVRIG, anti-TIGIT, and/or anti-PVRIG/anti-TIGIT bispecific antibody is labeled as described herein. In addition, the composition of therapeutic and diagnostic anti-PVRIG, anti-TIGIT, and/or anti-PVRIG/anti-TIGIT bispecific antibodies can also be co-administered with other drugs as outlined herein.

Particularly useful anti-PVRIG/anti-TIGIT bispecific antibodies for use in diagnosis include, but are not limited to these enumerated antibodies, or antibodies that utilize the CDRs with variant sequences, or those that compete for binding with any of the antibodies in FIGS. 5A-5H, 7A-7AE, and/or 23A-23D.

In many embodiments, a diagnostic anti-PVRIG, anti-TIGIT, and/or anti-PVRIG/anti-TIGIT bispecific antibody is labeled. By “labeled” herein is meant that the anti-PVRIG, anti-TIGIT, and/or anti-PVRIG/anti-TIGIT bispecific antibodies disclosed herein have one or more elements, isotopes, or chemical compounds attached to enable the detection in a screen or diagnostic procedure. In general, labels fall into several classes: a) immune labels, which may be an epitope incorporated as a fusion partner that is recognized by an antibody, b) isotopic labels, which may be radioactive or heavy isotopes, c) small molecule labels, which may include fluorescent and colorimetric dyes, or molecules such as biotin that enable other labeling methods, and d) labels such as particles (including bubbles for ultrasound labeling) or paramagnetic labels that allow body imagining. Labels may be incorporated into the anti-PVRIG/anti-TIGIT bispecific antibodies at any position and may be incorporated in vitro or in vivo during protein expression, as is known in the art.

Diagnosis can be done either in vivo, by administration of a diagnostic antibody that allows whole body imaging as described below, or in vitro, on samples removed from a patient. “Sample” in this context includes any number of things, including, but not limited to, bodily fluids (including, but not limited to, blood, urine, serum, lymph, saliva, anal and vaginal secretions, perspiration and semen), as well as tissue samples such as result from biopsies of relevant tissues.

In addition, as outlined below and in the Examples and Figures, information regarding the protein expression levels of either PVRIG and/or TIGIT, can be used to determine which antibodies should be administered to a patient.

B. Cancer Treatment

The anti-PVRIG, anti-TIGIT, and/or anti-PVRIG/anti-TIGIT bispecific antibodies of the invention find particular use in the treatment of cancer as a monotherapy. Due to the nature of an immuno-oncology mechanism of action, PVRIG and/or TIGIT do not necessarily need to be overexpressed on or correlated with a particular cancer type; that is, the goal is to have the anti-PVRIG/anti-TIGIT bispecific antibodies de-suppress T cell and NK cell activation, such that the immune system will go after the cancers.

In some embodiments, an anti-PVRIG, anti-TIGIT, and/or anti-PVRIG/anti-TIGIT bispecific antibodies comprising the anti-PVRIG antibody sequences of FIGS. 4A-4AA, 5A-5H, 7A-7AE, 11A-11I, 12A-12E, 13, 14A-14BX, 15A-15B, 16A-16E, 17A-17C, 26A-26B, and/or 40A-40T, find use in the treatment of cancer (including the activation of T cells and/or NK cells as outlined below), in particular those comprising and S241P substitution. While any anti-PVRIG, anti-TIGIT, and/or anti-PVRIG/anti-TIGIT bispecific antibodies comprising the anti-PVRIG antibody sequences of FIGS. 4A-4AA, 5A-5H, 7A-7AE, 11A-11I, 12A-12E, 13, 14A-14BX, 15A-15B, 16A-16E, 17A-17C, 26A-26B, and/or 40A-40T, find use in the treatment of cancer (including the activation of T cells and/or NK cells as outlined below), anti-PVRIG/anti-TIGIT bispecific antibodies comprising CHA.7.518.1, CHA.7.518.4, and/or CHA.7.538.2. find particular use in some embodiments.

While any anti-PVRIG/anti-TIGIT bispecific antibodies comprising the anti-TIGIT antibody sequences of FIGS. 23A-23D, 24A-24EE, and/or 41A-41T find use in the treatment of cancer (including the activation of T cells/and or NK cells as outlined below), anti-PVRIG/anti-TIGIT bispecific antibodies comprising CPA.9.086.H4(S241P), CPA.9.083.H4(S241P), CHA.9.547.7.H4(S241P), and CHA.9.547.13.H4(S241P), CHA.9.547.18 find particular use in some embodiments.

In some embodiments, the present invention also provides anti-PVRIG antibodies comprising the anti-PVRIG antibody sequences of FIGS. 4A-4AA, 5A-5H, 7A-7AE, 11A-11I, 12A-12E, 13, 14A-14BX, 15A-15B, 16A-16E, 17A-17C, 26A-26B, and/or 40A-40T, which find use in the treatment of cancer (including the activation of T cells/and or NK cells as outlined below). In some embodiments, the anti-PVRIG antibodies comprise:

Antibody sequence for PVRIG (IMGT CDR
definition bold and underlined)
CHA.7.518.4 VH

(SEQ ID NO: 3179)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
and
CHA.7.518.4 VL

(SEQ ID NO: 3180)

DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIK.

The anti-PVRIG/anti-TIGIT bispecific antibodies antibodies of the invention find particular use in the treatment of cancer. In general, the anti-PVRIG/anti-TIGIT bispecific antibodies antibodies of the invention are immunomodulatory, in that rather than directly attack cancerous cells, the antibodies of the invention stimulate the immune system, generally by inhibiting the action of the checkpoint receptor (e.g., PVRIG or TIGIT). Thus, unlike tumor-targeted therapies, which are aimed at inhibiting molecular pathways that are crucial for tumor growth and development, and/or depleting tumor cells, cancer immunotherapy is aimed to stimulate the patient's own immune system to eliminate cancer cells, providing long-lived tumor destruction. Various approaches can be used in cancer immunotherapy, among them are therapeutic cancer vaccines to induce tumor-specific T cell responses, and immunostimulatory antibodies (e.g., antagonists of inhibitory receptors=immune checkpoints) to remove immunosuppressive pathways.

Clinical responses with targeted therapy or conventional anti-cancer therapies tend to be transient as cancer cells develop resistance, and tumor recurrence takes place. However, the clinical use of cancer immunotherapy in the past few years has shown that this type of therapy can have durable clinical responses, showing dramatic impact on long term survival. However, although responses are long term, only a small number of patients respond (as opposed to conventional or targeted therapy, where a large number of patients respond, but responses are transient).

By the time a tumor is detected clinically, it has already evaded the immune-defense system by acquiring immunoresistant and immunosuppressive properties and creating an immunosuppressive tumor microenvironment through various mechanisms and a variety of immune cells.

Accordingly, the anti-PVRIG, anti-TIGIT, and/or anti-PVRIG/anti-TIGIT bispecific antibodies antibodies of the invention are useful in treating cancer. Due to the nature of an immuno-oncology mechanism of action, the checkpoint receptor (TIGIT or PVRIG) does not necessarily need to be overexpressed on or correlated with a particular cancer type; that is, the goal is to have the antibodies de-suppress T cell and NK cell activation, such that the immune system will go after the cancers.

“Cancer,” as used herein, refers broadly to any neoplastic disease (whether invasive or metastatic) characterized by abnormal and uncontrolled cell division causing malignant growth or tumor (e.g., unregulated cell growth.) The term “cancer” or “cancerous” as used herein should be understood to encompass any neoplastic disease (whether invasive, non-invasive or metastatic) which is characterized by abnormal and uncontrolled cell division causing malignant growth or tumor, non-limiting examples of which are described herein. This includes any physiological condition in mammals that is typically characterized by unregulated cell growth. Examples of cancer are exemplified in the working examples and also are described within the specification.

Non-limiting examples of cancer that can be treated using the anti-PVRIG/anti-TIGIT bispecific antibodies antibodies of the invention include, but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), esophageal cancer, melanoma, mesothelioma, merkel cell cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, prostate cancer, vulval cancer, thyroid cancer, hepatic carcinoma and various types of head and neck cancer, larynx cancer, oral cavity cancer, urothelial cancer, KRAS mutant tumors, Myelodysplastic syndromes (MDS), as well as B-cell malignancies, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenström's Macroglobulinemia); chronic lymphocytic leukemia (CLL); acute lymphoblastic leukemia (ALL); Hairy cell leukemia; chronic myeloblastic leukemia; adult T-cell leukemia/lymphoma; myeloma; multiple myeloma and post-transplant lymphoproliferative disorder (PTLD), lymphoid malignancies, abnormal vascular proliferation associated with phakomatoses, edema (such as that associated with brain tumors), and Meigs' syndrome, rectal cancer, renal cell cancer, soft-tissue sarcoma, Kaposi's sarcoma, carcinoid carcinoma, ovarian early or advanced (including metastatic).

As shown in the Examples of WO2016/134333, PVRIG is over expressed and/or correlates with tumor lymphocyte infiltration (as demonstrated by correlation to CD3, CD4, CD8 and PD-1 expression) in a number of different tumors of various origins, and thus is useful in treating any cancer, including but not limited to, prostate cancer, liver cancer (HCC), colorectal cancer, ovarian cancer, endometrial cancer, breast cancer, pancreatic cancer, stomach cancer, cervical cancer, head and neck cancer, thyroid cancer, testis cancer, urothelial cancer, lung cancer, melanoma, non melanoma skin cancer (squamous and basal cell carcinoma), glioma, renal cancer (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, and esophageal cancer.

In particular, anti-PVRIG/anti-TIGIT bispecific antibodies comprising CHA.7.518.1 as the PVRIG binding portion may find use in treating prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung cancer, non-small cell lung cancer), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, bladder cancer, Merkel Cells cancer, MSI-high cancer, KRAS mutant tumors, adult T-cell leukemia/lymphoma, myeloma and Myelodysplastic syndromes (MDS) esophageal cancer. In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS).

In particular, anti-PVRIG/anti-TIGIT bispecific antibodies comprising CHA.7.538.1.2 as the PVRIG binding portion may find use in treating prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung cancer, non-small cell lung cancer), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, bladder cancer, Merkel Cells cancer, MSI-high cancer, KRAS mutant tumors, adult T-cell leukemia/lymphoma, myeloma and Myelodysplastic syndromes (MDS) esophageal cancer. In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS).

In particular, anti-PVRIG/anti-TIGIT bispecific antibodies comprising CHA.7.518.4 as the PVRIG binding portion may find use in treating prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung cancer, non-small cell lung cancer), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, bladder cancer, Merkel Cells cancer, MSI-high cancer, KRAS mutant tumors, adult T-cell leukemia/lymphoma, myeloma and Myelodysplastic syndromes (MDS) esophageal cancer. In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS).

In particular, anti-PVRIG/anti-TIGIT bispecific antibodies comprising CPA.9.086 as the TIGIT binding portion may find use in treating prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung cancer, non-small cell lung cancer), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, bladder cancer, Merkel Cells cancer, MSI-high cancer, KRAS mutant tumors, adult T-cell leukemia/lymphoma, myeloma and Myelodysplastic syndromes (MDS) esophageal cancer. In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS).

In particular, anti-PVRIG/anti-TIGIT bispecific antibodies comprising CPA.9.083 as the TIGIT binding portion may find use in treating prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung cancer, non-small cell lung cancer), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, bladder cancer, Merkel Cells cancer, MSI-high cancer, KRAS mutant tumors, adult T-cell leukemia/lymphoma, myeloma and Myelodysplastic syndromes (MDS) esophageal cancer. In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS).

In particular, anti-PVRIG/anti-TIGIT bispecific antibodies comprising CHA.9.547.7 as the TIGIT binding portion may find use in treating prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung cancer, non-small cell lung cancer), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, bladder cancer, Merkel Cells cancer, MSI-high cancer, KRAS mutant tumors, adult T-cell leukemia/lymphoma, myeloma and Myelodysplastic syndromes (MDS) esophageal cancer. In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS).

In particular, anti-PVRIG/anti-TIGIT bispecific antibodies comprising CHA.9.547.13 as the TIGIT binding portion may find use in treating prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung cancer, non-small cell lung cancer), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, bladder cancer, Merkel Cells cancer, MSI-high cancer, KRAS mutant tumors, adult T-cell leukemia/lymphoma, myeloma and Myelodysplastic syndromes (MDS) esophageal cancer. In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS).

In particular, anti-PVRIG/anti-TIGIT bispecific antibodies comprising CPA.9.547.18 as the TIGIT binding portion may find use in treating prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung cancer, non-small cell lung cancer), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, bladder cancer, Merkel Cells cancer, MSI-high cancer, KRAS mutant tumors, adult T-cell leukemia/lymphoma, myeloma and Myelodysplastic syndromes (MDS) esophageal cancer. In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS).

In particular, anti-PVRIG/anti-TIGIT bispecific antibodies comprising the following sequences may find use in treating prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung cancer, non-small cell lung cancer), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, bladder cancer, esophageal cancer Merkel Cells cancer, MSI-high cancer, KRAS mutant tumors, adult T-cell leukemia/lymphoma, myeloma and Myelodysplastic syndromes (MDS), wherein the anti-PVRIG/anti-TIGIT bispecific comprises the following sequences:

CHA.7.518.1.H4(S241P) VH IgG1 CH1 IgG4 CH2—CH3
S354C, E356D, M358L, T366W

(SEQ ID NO: 3175)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPCQDELTKNQVSLWCLVKGFYPSDIAVEWESNGQPENNYKTTPPVLDS
DGSFFLYSRLTVDKSRWQEGNVFSCSVMHEALHNHYTQKSLSLSPGK;
CHA.7.518.1.H4(S241P) VL Ckappa

(SEQ ID NO: 3176)

DIQMTQSPSSLSASVGDRVTITCRVSENIYSNLAWYQQKPGKAPKLLIYE
ATNLAEGVPSRFSGSGSGTDFTLTISSLQPEDFATYYCQHFWGTPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC;
and
CPA.9.086 HC Clambda Crossmab IgG4 Y349C, E356D,
M358L, T366S, L368A, Y407V

(SEQ ID NO: 3177)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSASGQPKAAPSVTLFPPSSEELQANKATLV
CLISDFYPGAVTVAWKADSSPVKAGVETTTPSKQSNNKYAASSYLSLTPE
QWKSHRSYSCQVTHEGSTVEKTVAPTECSESKYGPPCPPCPAPEFLGGPS
VFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKA
KGQPREPQVCTLPPSQDELTKNQVSLSCAVKGFYPSDIAVEWESNGQPEN
NYKTTPPVLDSDGSFFLVSRLTVDKSRWQEGNVFSCSVMHEALHNHYTQK
SLSLSPGK;
CPA.9.086 LC CH1

(SEQ ID NO: 3178)

QSALTQPRSASGNPGQRVTISCSGSSSNMGRRPVNWYQQIPGTAPKLLIY
SQNQRPSGVPDRFSGSQSGTSASLTISGLQSEDEAEYFCAVWDDIGRVLQ
LGGGTQLAVLSSASTKGPSVFPLAPSSKSTSGGTAALGCLVKDYFPEPVT
VSWNSGALTSGVHTFPAVLQSSGLYSLSSVVTVPSSSLGTQTYICNVNHK
PSNTKVDKKVEPKSC.

In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS).

In particular, anti-PVRIG/anti-TIGIT bispecific antibodies comprising the following sequences may find use in treating prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung cancer, non-small cell lung cancer), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, bladder cancer, esophageal cancer Merkel Cells cancer, MSI-high cancer, KRAS mutant tumors, adult T-cell leukemia/lymphoma, myeloma and Myelodysplastic syndromes (MDS), wherein the anti-PVRIG/anti-TIGIT bispecific includes any one of the following anti-PVRIG/anti-TIGIT bispecific antibodies:

an anti-PVRIG/anti-TIGIT bispecific
comprising the following sequences:
CHA.7.518.1 VH

(SEQ ID NO: 1539)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.1 VL

(SEQ ID NO: 1544)

DIQMTQSPSSLSASVGDRVTITCRVSENIYSNLAWYQQKPGKAPKLLIYE
ATNLAEGVPSRFSGSGSGTDFTLTISSLQPEDFATYYCQHFWGTPYTFGQ
GTKLEIK;
and
CPA.9.086 VH

(SEQ ID NO: 1634)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSS;
CPA.9.086 VL

(SEQ ID NO: 1639)

QSALTQPRSASGNPGQRVTISCSGSSSNMGRRPVNWYQQIPGTAPKLLIY
SQNQRPSGVPDRFSGSQSGTSASLTISGLQSEDEAEYFCAVWDDIGRVLQ
LGGGTQLAVL;
OR
an anti-PVRIG/anti-TIGIT bispecific
comprising the following sequences:
CHA.7.518.4 VH

(SEQ ID NO: 3179)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.4 VL

(SEQ ID NO: 3180)

DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIK;
And
CPA.9.086 VH

(SEQ ID NO: 1634)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSS;
CPA.9.086 VL

(SEQ ID NO: 1639)

QSALTQPRSASGNPGQRVTISCSGSSSNMGRRPVNWYQQIPGTAPKLLIY
SQNQRPSGVPDRFSGSQSGTSASLTISGLQSEDEAEYFCAVWDDIGRVLQ
LGGGTQLAVL;
OR
an anti-PVRIG/anti-TIGIT bispecific
comprising the following sequences:
CHA.7.518.1 VH

(SEQ ID NO: 1539)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.1 VL

(SEQ ID NO: 1544)

DIQMTQSPSSLSASVGDRVTITCRVSENIYSNLAWYQQKPGKAPKLLIYE
ATNLAEGVPSRFSGSGSGTDFTLTISSLQPEDFATYYCQHFWGTPYTFGQ
GTKLEIK;
and
CHA.9.547.18 VH

(SEQ ID NO: 1664)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSS;
CHA.9.547.18 VL

(SEQ ID NO: 1668)

DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIK;
OR
an anti-PVRIG/anti-TIGIT bispecific
comprising the following sequences:
CHA.7.518.4 VH

(SEQ ID NO: 3179)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.4 VL

(SEQ ID NO: 3180)

DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIK;
and
CHA.9.547.18 VH

(SEQ ID NO: 1664)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSS;
CHA.9.547.18 VL

(SEQ ID NO: 1668)

DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIK.

In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS).

In particular, anti-PVRIG antibodies comprising the following sequences may find use in treating prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung cancer, non-small cell lung cancer), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, bladder cancer, esophageal cancer Merkel Cells cancer, MSI-high cancer, KRAS mutant tumors, adult T-cell leukemia/lymphoma, myeloma and Myelodysplastic syndromes (MDS), wherein the anti-PVRIG/anti-TIGIT bispecific includes any one of the following anti-PVRIG/anti-TIGIT bispecific antibodies:

an anti-PVRIG/anti-TIGIT bispecific
comprising the following sequences:
CHA.7.518.1 VH

(SEQ ID NO: 1539)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.1 VL

(SEQ ID NO: 1544)

DIQMTQSPSSLSASVGDRVTITCRVSENIYSNLAWYQQKPGKAPKLLIYE
ATNLAEGVPSRFSGSGSGTDFTLTISSLQPEDFATYYCQHFWGTPYTFGQ
GTKLEIK;
and
CPA.9.086 VH

(SEQ ID NO: 1634)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSS;
CPA.9.086 VL

(SEQ ID NO: 1639)

QSALTQPRSASGNPGQRVTISCSGSSSNMGRRPVNWYQQIPGTAPKLLIY
SQNQRPSGVPDRFSGSQSGTSASLTISGLQSEDEAEYFCAVWDDIGRVLQ
LGGGTQLAVL;
OR
an anti-PVRIG/anti-TIGIT bispecific
comprising the following sequences:
CHA.7.518.4 VH

(SEQ ID NO: 3179)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.4 VL

(SEQ ID NO: 3180)

DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIK;
and
CPA.9.086 VH

(SEQ ID NO: 1634)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSS;
CPA.9.086 VL

(SEQ ID NO: 1639)

QSALTQPRSASGNPGQRVTISCSGSSSNMGRRPVNWYQQIPGTAPKLLIY
SQNQRPSGVPDRFSGSQSGTSASLTISGLQSEDEAEYFCAVWDDIGRVLQ
LGGGTQLAVL;
OR
an anti-PVRIG/anti-TIGIT bispecific
comprising the following sequences:
CHA.7.518.1 VH

(SEQ ID NO: 1539)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.1 VL

(SEQ ID NO: 1544)

DIQMTQSPSSLSASVGDRVTITCRVSENIYSNLAWYQQKPGKAPKLLIYE
ATNLAEGVPSRFSGSGSGTDFTLTISSLQPEDFATYYCQHFWGTPYTFGQ
GTKLEIK;
and
CHA.9.547.18 VH

(SEQ ID NO: 1664)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSS;
CHA.9.547.18 VL

(SEQ ID NO: 1668)

DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIK;
OR
an anti-PVRIG/anti-TIGIT bispecific
comprising the following sequences:
CHA.7.518.4 VH

(SEQ ID NO: 3179)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.4 VL

(SEQ ID NO: 3180)

DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIK;
and
CHA.9.547.18 VH

(SEQ ID NO: 1664)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSS;
CHA.9.547.18 VL

(SEQ ID NO: 1668)

DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIK.

In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS).

In particular, anti-PVRIG/anti-TIGIT bispecific antibodies comprising the following sequences may find use in treating prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung cancer, non-small cell lung cancer), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, bladder cancer, esophageal cancer Merkel Cells cancer, MSI-high cancer, KRAS mutant tumors, adult T-cell leukemia/lymphoma, myeloma and Myelodysplastic syndromes (MDS), wherein the anti-PVRIG/anti-TIGIT bispecific includes any one of the following anti-PVRIG/anti-TIGIT bispecific antibodies:

an anti-PVRIG/anti-TIGIT bispecific
comprising the following sequences:
CHA.7.518.1 VH

(SEQ ID NO: 1539)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.1 VL

(SEQ ID NO: 1544)

DIQMTQSPSSLSASVGDRVTITCRVSENIYSNLAWYQQKPGKAPKLLIYE
ATNLAEGVPSRFSGSGSGTDFTLTISSLQPEDFATYYCQHFWGTPYTFGQ
GTKLEIK;
and
CPA.9.086 VH

(SEQ ID NO: 1634)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSS;
CPA.9.086 VL

(SEQ ID NO: 1639)

QSALTQPRSASGNPGQRVTISCSGSSSNMGRRPVNWYQQIPGTAPKLLIY
SQNQRPSGVPDRFSGSQSGTSASLTISGLQSEDEAEYFCAVWDDIGRVLQ
LGGGTQLAVL;
OR
an anti-PVRIG/anti-TIGIT bispecific
comprising the following sequences:
CHA.7.518.4 VH

(SEQ ID NO: 3179)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.4 VL

(SEQ ID NO: 3180)

DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIK;
And
CPA.9.086 VH

(SEQ ID NO: 1634)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSS;
CPA.9.086 VL

(SEQ ID NO: 1639)

QSALTQPRSASGNPGQRVTISCSGSSSNMGRRPVNWYQQIPGTAPKLLIY
SQNQRPSGVPDRFSGSQSGTSASLTISGLQSEDEAEYFCAVWDDIGRVLQ
LGGGTQLAVL;
OR
an anti-PVRIG/anti-TIGIT bispecific
comprising the following sequences:
CHA.7.518.1 VH

(SEQ ID NO: 1539)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.1 VL

(SEQ ID NO: 1544)

DIQMTQSPSSLSASVGDRVTITCRVSENIYSNLAWYQQKPGKAPKLLIYE
ATNLAEGVPSRFSGSGSGTDFTLTISSLQPEDFATYYCQHFWGTPYTFGQ
GTKLEIK;
and
CHA.9.547.18 VH

(SEQ ID NO: 1664)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSS;
CHA.9.547.18 VL

(SEQ ID NO: 1668)

DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIK;
OR
an anti-PVRIG/anti-TIGIT bispecific
comprising the following sequences:
CHA.7.518.4 VH

(SEQ ID NO: 3179)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.4 LC

(SEQ ID NO: 3180)

DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIK;
and
CHA.9.547.18 VH

(SEQ ID NO: 1664)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSS;
CHA.9.547.18 VL

(SEQ ID NO: 1668)

DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIK.

In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS).

In particular, anti-PVRIG antibodies comprising the following sequences may find use in treating prostate cancer, liver cancer (HCC), colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung cancer, non-small cell lung cancer), melanoma, non melanoma skin cancer (squamous and basal cell carcinoma), glioma, renal cancer (RCC), lymphoma (NHL or HL), Acute myeloid leukemia (AML), T cell Acute Lymphoblastic Leukemia (T-ALL), Diffuse Large B cell lymphoma, testicular germ cell tumors, mesothelioma, bladder cancer, esophageal cancer Merkel Cells cancer, MSI-high cancer, KRAS mutant tumors, adult T-cell leukemia/lymphoma, myeloma and Myelodysplastic syndromes (MDS):

an anti-PVRIG antibody comprising
the following sequences:
CHA.7.518.4 VH

(SEQ ID NO: 3179)

(SEQQVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS
and
CHA.7.518.4 VL

(SEQ ID NO: 3180)

(SEQDIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIK.

In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS).

C. Combination Therapies

As is known in the art, combination therapies comprising a therapeutic anti-PVRIG, anti-TIGIT, and/or anti-PVRIG/anti-TIGIT bispecific antibody targeting an immunotherapy target and an additional therapeutic agent, specific for the disease condition, are showing great promise. For example, in the area of immunotherapy, there are a number of promising combination therapies using a chemotherapeutic agent (either a small molecule drug or an anti-tumor antibody) or with an immuno-oncology antibody (for example, an anti-PVRIG/anti-TIGIT bispecific antibody of the invention).

The terms “in combination with” and “co-administration” are not limited to the administration of the prophylactic or therapeutic agents at exactly the same time. Instead, it is meant that the antibody and the other agent or agents are administered in a sequence and within a time interval such that they may act together to provide a benefit that is increased versus treatment with only either the antibody of the present invention or the other agent or agents. It is preferred that the antibody and the other agent or agents act additively, and especially preferred that they act synergistically. Such molecules are suitably present in combination in amounts that are effective for the purpose intended. The skilled medical practitioner can determine empirically, or by considering the pharmacokinetics and modes of action of the agents, the appropriate dose or doses of each therapeutic agent, as well as the appropriate timings and methods of administration.

Accordingly, the anti-PVRIG/anti-TIGIT bispecific antibodies of the present invention may be administered concomitantly with one or more other therapeutic regimens or agents. The additional therapeutic regimes or agents may be used to improve the efficacy or safety of the anti-PVRIG, anti-TIGIT, and/or anti-PVRIG/anti-TIGIT bispecific antibody. Also, the additional therapeutic regimes or agents may be used to treat the same disease or a comorbidity rather than to alter the action of the anti-PVRIG/anti-TIGIT bispecific antibody. For example, an anti-PVRIG, anti-TIGIT, and/or anti-PVRIG/anti-TIGIT bispecific antibody of the present invention may be administered to the patient along with chemotherapy, radiation therapy, or both chemotherapy and radiation therapy.

1. PVRIG, TIGIT, and/or PVRIG/TIGIT Bispecific Antibodies with Chemotherapeutic Small Molecules

The anti-PVRIG/anti-TIGIT bispecific antibodies of the present invention may be administered in combination with one or more other prophylactic or therapeutic agents, including but not limited to cytotoxic agents, chemotherapeutic agents, cytokines, growth inhibitory agents, anti-hormonal agents, kinase inhibitors, anti-angiogenic agents, cardioprotectants, immunostimulatory agents, immunosuppressive agents, agents that promote proliferation of hematological cells, angiogenesis inhibitors, protein tyrosine kinase (PTK) inhibitors, or other therapeutic agents.

In this context, a “chemotherapeutic agent” is a chemical compound useful in the treatment of cancer. Examples of chemotherapeutic agents include alkylating agents such as thiotepa and cyclosphosphamide, alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); delta-9-tetrahydrocannabinol (dronabinol, MARINOL′); beta-lapachone; lapachol; colchicines; betulinic acid; a camptothecin (including the synthetic analogue topotecan (HYCAMTN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®; Bristol-Myers Squibb Oncology, Princeton, N.J.), ABRAXANE®, cremophor-free, albumin-engineered nanoparticle formulation of paclitaxel (American Pharmaceutical Partners, Schaumberg, Ill.), and docetaxel (TAXOTERE®; Rhone-Poulenc Rorer, Antony, France); chloranbucil; gemcitabine (GEMZARM®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone; CVP, an abbreviation for a combined therapy of cyclophosphamide, vincristine, and prednisolone; and FOLFOX, an abbreviation for a treatment regimen with oxaliplatin (ELOXATIN®) combined with 5-FU and leucovorin.

According to at least some embodiments, the anti-PVRIG/anti-TIGIT bispecific antibodies of the inventioncould be used in combination with any of the known in the art standard of care cancer treatment (as can be found, for example, in http://www.cancer.gov/cancertopics).

Thus, in some cases, the anti-PVRIG/anti-TIGIT bispecific antibodies outlined herein (particularly those including CHA.7.538.1.2 and/or CHA.7.518.1 as the PVRIG binding portion) can be combined with chemotherapeutic agents. Similarly, the anti-PVRIG/anti-TIGIT bispecific outlined herein (particularly those including CPA.9.086, CPA.9.083 and/or CHA.9.547.13 as the TIGIT binding portion) can be combined with chemotherapeutic agents.

In some embodiments, the anti-PVRIG/anti-TIGIT bispecific antibody that can be combined with chemotherapeutic agents comprises the following sequences:

CHA.7.518.1.H4(S241P) VH IgG1 CH1 IgG4 CH2—CH3
S354C, E356D, M358L, T366W

(SEQ ID NO: 3175)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPCQDELTKNQVSLWCLVKGFYPSDIAVEWESNGQPENNYKTTPPVLDS
DGSFFLYSRLTVDKSRWQEGNVFSCSVMHEALHNHYTQKSLSLSPGK
CHA.7.518.1.H4(S241P) VL Ckappa

(SEQ ID NO: 3176)

DIQMTQSPSSLSASVGDRVTITCRVSENIYSNLAWYQQKPGKAPKLLIYE
ATNLAEGVPSRFSGSGSGTDFTLTISSLQPEDFATYYCQHFWGTPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
CPA.9.086 HC Clambda Crossmab IgG4 Y349C, E356D,
M358L, T366S, L368A, Y407V

(SEQ ID NO: 3177)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSSASGQPKAAPSVTLFPPSSEELQANKATLV
CLISDFYPGAVTVAWKADSSPVKAGVETTTPSKQSNNKYAASSYLSLTPE
QWKSHRSYSCQVTHEGSTVEKTVAPTECSESKYGPPCPPCPAPEFLGGPS
VFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKA
KGQPREPQVCTLPPSQDELTKNQVSLSCAVKGFYPSDIAVEWESNGQPEN
NYKTTPPVLDSDGSFFLVSRLTVDKSRWQEGNVFSCSVMHEALHNHYTQK
SLSLSPGK
CPA.9.086 LC CH1

(SEQ ID NO: 3178)

QSALTQPRSASGNPGQRVTISCSGSSSNMGRRPVNWYQQIPGTAPKLLIY
SQNQRPSGVPDRFSGSQSGTSASLTISGLQSEDEAEYFCAVWDDIGRVLQ
LGGGTQLAVLSSASTKGPSVFPLAPSSKSTSGGTAALGCLVKDYFPEPVT
VSWNSGALTSGVHTFPAVLQSSGLYSLSSVVTVPSSSLGTQTYICNVNHK
PSNTKVDKKVEPKSC

In some embodiments, the anti-PVRIG/anti-TIGIT bispecific antibody that can be combined with chemotherapeutic agents comprises the following sequences:

Antibody sequence for PVRIG (IMGT CDR
definition bold and underlined)
CHA.7.518.4 VH

(SEQ ID NO: 3179)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS;
CHA.7.518.4 VL

(SEQ ID NO: 3180)

DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIK;
And
Antibody sequence for TIGIT (IMGT CDR
definition bold and underlined)
CHA.9.547.18 VH

(SEQ ID NO: 1664)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSS;
CHA.9.547.18 VL

(SEQ ID NO: 1668)

DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIK.

In some embodiments, the anti-PVRIG/anti-TIGIT bispecific antibody that can be combined with chemotherapeutic agents comprises the following sequences:

CHA.7.518.1 VH

(SEQ ID NO: 1539)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS
CHA.7.518.1 VL

(SEQ ID NO: 1544)

DIQMTQSPSSLSASVGDRVTITCRVSENIYSNLAWYQQKPGKAPKLLIYE
ATNLAEGVPSRFSGSGSGTDFTLTISSLQPEDFATYYCQHFWGTPYTFGQ
GTKLEIK
And
CPA.9.086 VH

(SEQ ID NO: 1634)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSS
CPA.9.086 VL

(SEQ ID NO: 1639)

QSALTQPRSASGNPGQRVTISCSGSSSNMGRRPVNWYQQIPGTAPKLLIY
SQNQRPSGVPDRFSGSQSGTSASLTISGLQSEDEAEYFCAVWDDIGRVLQ
LGGGTQLAVL.

In some embodiments, the anti-PVRIG/anti-TIGIT bispecific antibody that can be combined with chemotherapeutic agents comprises the following sequences:

CHA.7.518.4 VH

(SEQ ID NO: 3179)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS
CHA.7.518.4 VL

(SEQ ID NO: 3180)

DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIK
And
CPA.9.086 VH

(SEQ ID NO: 1634)

EVQLVETGGGLIQPGRSLRLSCAASGFTFSSYAMHWVRQAPGKGLEWVAV
ISYAGEVKYYADSVKGRFTISRDNSKNTLYLQMNSLRAEDTAVYYCARDP
LPLHYYGMDVWGQGTTVTVSS
CPA.9.086 VL

(SEQ ID NO: 1639)

QSALTQPRSASGNPGQRVTISCSGSSSNMGRRPVNWYQQIPGTAPKLLIY
SQNQRPSGVPDRFSGSQSGTSASLTISGLQSEDEAEYFCAVWDDIGRVLQ
LGGGTQLAVL.

In some embodiments, the anti-PVRIG/anti-TIGIT bispecific antibody that can be combined with chemotherapeutic agents comprises the following sequences:

CHA.7.518.1 VH

(SEQ ID NO: 1539)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS
CHA.7.518.1 VL

(SEQ ID NO: 1544)

DIQMTQSPSSLSASVGDRVTITCRVSENIYSNLAWYQQKPGKAPKLLIYE
ATNLAEGVPSRFSGSGSGTDFTLTISSLQPEDFATYYCQHFWGTPYTFGQ
GTKLEIK
And
CHA.9.547.18 VH

(SEQ ID NO: 1664)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSS
CHA.9.547.18 VL

(SEQ ID NO: 1668)

DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIK.

In some embodiments, the anti-PVRIG/anti-TIGIT bispecific antibody that can be combined with chemotherapeutic agents comprises the following sequences:

CHA.7.518.4 VH

(SEQ ID NO: 3179)

QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS
CHA.7.518.4 VL

(SEQ ID NO: 3180)

DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTFGQ
GTKLEIK
And
CHA.9.547.18 VH

(SEQ ID NO: 1664)

EVQLVESGGGLVQPGGSLRLSCAASGFTFSSYIMSWVRQAPGKGLEWVAT
ISGGSKGQYYADSVKGRFTISRDNSKNTLYLQMNSLRAEDTAVYYCAKWL
LSYYAMDYWGQGTLVTVSS
CHA.9.547.18 VL

(SEQ ID NO: 1668)

DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFGQ
GTKLEIK.

D. Assessment of Treatment

Generally, the anti-PVRIG, anti-TIGIT, and/or anti-PVRIG/anti-TIGIT bispecific antibodies of the invention, are administered to patients with cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays. For example, evaluation of changes in immune status (e.g. presence of ICOS+CD4+ T cells following ipi treatment) along with “old fashioned” measurements such as tumor burden, size, invasiveness, LN involvement, metastasis, etc. can be done. Thus, any or all of the following can be evaluated: the inhibitory effects of PVRIG or TIGIT on CD4⁺ T cell activation or proliferation, CD8⁺ T (CTL) cell activation or proliferation, CD8⁺ T cell-mediated cytotoxic activity and/or CTL mediated cell depletion, NK cell activity and NK mediated cell depletion, the potentiating effects of PVRIG or TIGIT on Treg cell differentiation and proliferation and Treg- or myeloid derived suppressor cell (MDSC)-mediated immunosuppression or immune tolerance, and/or the effects of PVRIG or TIGIT on proinflammatory cytokine production by immune cells, e.g., IL-2, IFN-γ or TNF-α production by T or other immune cells.

In some embodiments, assessment of treatment is done by evaluating immune cell proliferation, using for example, CFSE dilution method, Ki67 intracellular staining of immune effector cells, and 3H-Thymidine incorporation method.

In some embodiments, assessment of treatment is done by evaluating the increase in gene expression or increased protein levels of activation-associated markers, including one or more of: CD25, CD69, CD137, ICOS, PD1, GITR, OX40, and cell degranulation measured by surface expression of CD107A.

In some embodiments, the assessment of treatment is done by assessing the amount of T cell proliferation in the absence of treatment, for example prior to administration of the antibodies of the invention. If, after administration, the patient has an increase in T cell proliferation, e.g. a subset of the patient's T cells are proliferating, this is an indication that the T cells were activated.

Similarly, assessment of treatment with the antibodies of the invention can be done by measuring the patient's IFNγ levels prior to administration and post-administration to assess efficacy of treatment. This may be done within hours or days.

In general, gene expression assays are done as is known in the art. See for example Goodkind et al., Computers and Chem. Eng. 29(3):589 (2005), Han et al., Bioinform. Biol. Insights 11/15/15 9(Suppl. 1):29-46, Campo et al., Nod. Pathol. 2013 January; 26 suppl. 1:S97-S110, the gene expression measurement techniques of which are expressly incorporated by reference herein.

In general, protein expression measurements are also similarly done as is known in the art, see for example, Wang et al., Recent Advances in Capillary Electrophoresis-Based Proteomic Techniques for Biomarker Discovery, Methods. Mol. Biol. 2013:984:1-12; Taylor et al, BioMed Res. Volume 2014, Article ID 361590, 8 pages, Becerk et al., Mutat. Res 2011 Jun. 17:722(2): 171-182, the measurement techniques of which are expressly incorporated herein by reference.

In some embodiments, assessment of treatment is done by assessing cytotoxic activity measured by target cell viability detection via estimating numerous cell parameters such as enzyme activity (including protease activity), cell membrane permeability, cell adherence, ATP production, co-enzyme production, and nucleotide uptake activity. Specific examples of these assays include, but are not limited to, Trypan Blue or PI staining, ⁵¹Cr or ³⁵S release method, LDH activity, MTT and/or WST assays, Calcein-AM assay, Luminescent based assay, and others.

In some embodiments, assessment of treatment is done by assessing T cell activity measured by cytokine production, measure either intracellularly in culture supernatant using cytokines including, but not limited to, IFNγ, TNFα, GM-CSF, IL2, IL6, IL4, IL5, IL10, IL13 using well known techniques.

Accordingly, assessment of treatment can be done using assays that evaluate one or more of the following: (i) increases in immune response, (ii) increases in activation of αβ and/or γδ T cells, (iii) increases in cytotoxic T cell activity, (iv) increases in NK and/or NKT cell activity, (v) alleviation of αβ and/or γδ T-cell suppression, (vi) increases in pro-inflammatory cytokine secretion, (vii) increases in IL-2 secretion; (viii) increases in interferon-γ production, (ix) increases in Th1 response, (x) decreases in Th2 response, (xi) decreases or eliminates cell number and/or activity of at least one of regulatory T cells (Tregs).

E. Assays to Measure Efficacy

In some embodiments, T cell activation is assessed using a Mixed Lymphocyte Reaction (MLR) assay as is described in the Examples. An increase in activity indicates immunostimulatory activity. Appropriate increases in activity are outlined below.

In one embodiment, the signaling pathway assay measures increases or decreases in immune response as measured for an example by phosphorylation or de-phosphorylation of different factors, or by measuring other post translational modifications. An increase in activity indicates immunostimulatory activity. Appropriate increases in activity are outlined below.

In one embodiment, the signaling pathway assay measures increases or decreases in activation of αβ and/or γδ T cells as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in cytotoxic T cell activity as measured for an example by direct killing of target cells like for an example cancer cells or by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in NK and/or NKT cell activity as measured for an example by direct killing of target cells like for an example cancer cells or by cytokine secretion or by changes in expression of activation markers like for an example CD107a, etc. An increase in activity indicates immunostimulatory activity. Appropriate increases in activity are outlined below.

In one embodiment, the signaling pathway assay measures increases or decreases in αβ and/or γδ T-cell suppression, as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in pro-inflammatory cytokine secretion as measured for example by ELISA or by Luminex or by Multiplex bead based methods or by intracellular staining and FACS analysis or by Alispot etc. An increase in activity indicates immunostimulatory activity. Appropriate increases in activity are outlined below.

In one embodiment, the signaling pathway assay measures increases or decreases in IL-2 secretion as measured for example by ELISA or by Luminex or by Multiplex bead based methods or by intracellular staining and FACS analysis or by Alispot etc. An increase in activity indicates immunostimulatory activity. Appropriate increases in activity are outlined below.

In one embodiment, the signaling pathway assay measures increases or decreases in interferon-γ production as measured for example by ELISA or by Luminex or by Multiplex bead based methods or by intracellular staining and FACS analysis or by Alispot etc. An increase in activity indicates immunostimulatory activity. Appropriate increases in activity are outlined below.

In one embodiment, the signaling pathway assay measures increases or decreases in Th1 response as measured for an example by cytokine secretion or by changes in expression of activation markers. An increase in activity indicates immunostimulatory activity. Appropriate increases in activity are outlined below.

In one embodiment, the signaling pathway assay measures increases or decreases in Th2 response as measured for an example by cytokine secretion or by changes in expression of activation markers. An increase in activity indicates immunostimulatory activity. Appropriate increases in activity are outlined below.

In one embodiment, the signaling pathway assay measures increases or decreases cell number and/or activity of at least one of regulatory T cells (Tregs), as measured for example by flow cytometry or by IHC. A decrease in response indicates immunostimulatory activity. Appropriate decreases are the same as for increases, outlined below.

In one embodiment, the signaling pathway assay measures increases or decreases in M2 macrophages cell numbers, as measured for example by flow cytometry or by IHC. A decrease in response indicates immunostimulatory activity. Appropriate decreases are the same as for increases, outlined below.

In one embodiment, the signaling pathway assay measures increases or decreases in M2 macrophage pro-tumorigenic activity, as measured for an example by cytokine secretion or by changes in expression of activation markers. A decrease in response indicates immunostimulatory activity. Appropriate decreases are the same as for increases, outlined below.

In one embodiment, the signaling pathway assay measures increases or decreases in N2 neutrophils increase, as measured for example by flow cytometry or by IHC. A decrease in response indicates immunostimulatory activity. Appropriate decreases are the same as for increases, outlined below.

In one embodiment, the signaling pathway assay measures increases or decreases in N2 neutrophils pro-tumorigenic activity, as measured for an example by cytokine secretion or by changes in expression of activation markers. A decrease in response indicates immunostimulatory activity. Appropriate decreases are the same as for increases, outlined below.

In one embodiment, the signaling pathway assay measures increases or decreases in inhibition of T cell activation, as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in inhibition of CTL activation as measured for an example by direct killing of target cells like for an example cancer cells or by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in αβ and/or γδ T cell exhaustion as measured for an example by changes in expression of activation markers. A decrease in response indicates immunostimulatory activity. Appropriate decreases are the same as for increases, outlined below.

In one embodiment, the signaling pathway assay measures increases or decreases αβ and/or γδ T cell response as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in stimulation of antigen-specific memory responses as measured for an example by cytokine secretion or by proliferation or by changes in expression of activation markers like for an example CD45RA, CCR7 etc. An increase in activity indicates immunostimulatory activity. Appropriate increases in activity are outlined below.

In one embodiment, the signaling pathway assay measures increases or decreases in apoptosis or lysis of cancer cells as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, the signaling pathway assay measures increases or decreases in stimulation of cytotoxic or cytostatic effect on cancer cells. as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, the signaling pathway assay measures increases or decreases direct killing of cancer cells as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, the signaling pathway assay measures increases or decreases Th17 activity as measured for an example by cytokine secretion or by proliferation or by changes in expression of activation markers. An increase in activity indicates immunostimulatory activity. Appropriate increases in activity are outlined below.

In one embodiment, the signaling pathway assay measures increases or decreases in induction of complement dependent cytotoxicity and/or antibody dependent cell-mediated cytotoxicity, as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, T cell activation is measured for an example by direct killing of target cells like for an example cancer cells or by cytokine secretion or by proliferation or by changes in expression of activation markers like for an example CD137, CD107a, PD1, etc. For T-cells, increases in proliferation, cell surface markers of activation (e.g., CD25, CD69, CD137, PD1), cytotoxicity (ability to kill target cells), and cytokine production (e.g., IL-2, IL-4, IL-6, IFNγ, TNF-a, IL-10, IL-17A) would be indicative of immune modulation that would be consistent with enhanced killing of cancer cells.

In one embodiment, NK cell activation is measured for example by direct killing of target cells like for an example cancer cells or by cytokine secretion or by changes in expression of activation markers like for an example CD107a, etc. For NK cells, increases in proliferation, cytotoxicity (ability to kill target cells and increases CD107a, granzyme, and perforin expression), cytokine production (e.g., IFNγ and TNF), and cell surface receptor expression (e.g., CD25) would be indicative of immune modulation that would be consistent with enhanced killing of cancer cells.

In one embodiment, γδ T cell activation is measured for example by cytokine secretion or by proliferation or by changes in expression of activation markers.

In one embodiment, Th1 cell activation is measured for example by cytokine secretion or by changes in expression of activation markers.

Appropriate increases in activity or response (or decreases, as appropriate as outlined above), are increases of at least about 5%, 10%, 20%, 30%, 40%, 50%, 60%, 70%, 80%, 90%, 95% or 98 to 99% percent over the signal in either a reference sample or in control samples, for example test samples that do not contain an anti-PVRIG/anti-TIGIT bispecific antibody of the invention.

Similarly, increases of at least one-, two-, three-, four- or five-fold as compared to reference or control samples show efficacy.

### X. EXAMPLES

### Example 1: Generation and Characterization of an Anti-PVRIG/Anti-TIGIT Bispecific Antibody

### 1. Introduction

PVRIG and TIGIT are inhibitory immune checkpoint receptors expressed on T cells and Natural Killer cells. Antibody-mediated blockade of the interaction of these receptors with their respective ligands results in enhanced T cell activity. The combination anti-PVRIG and anti-TIGIT antibody therapy results in synergistic or additive effect in in vitro functional assays. Here we generate and characterize an anti-PVRIG/anti-TIGIT bispecific antibody and show that the antibody retains the binding affinity of each arm for the respective target antigen. Furthermore, the PVRIG/TIGIT BsAb is equivalent to the anti-PVRIG and anti-TIGIT antibody combination in T cell based in vitro functional assay.

### 2. Protocols

Anti-PVRIG/Anti-TIGIT Bispecific Construct Design:

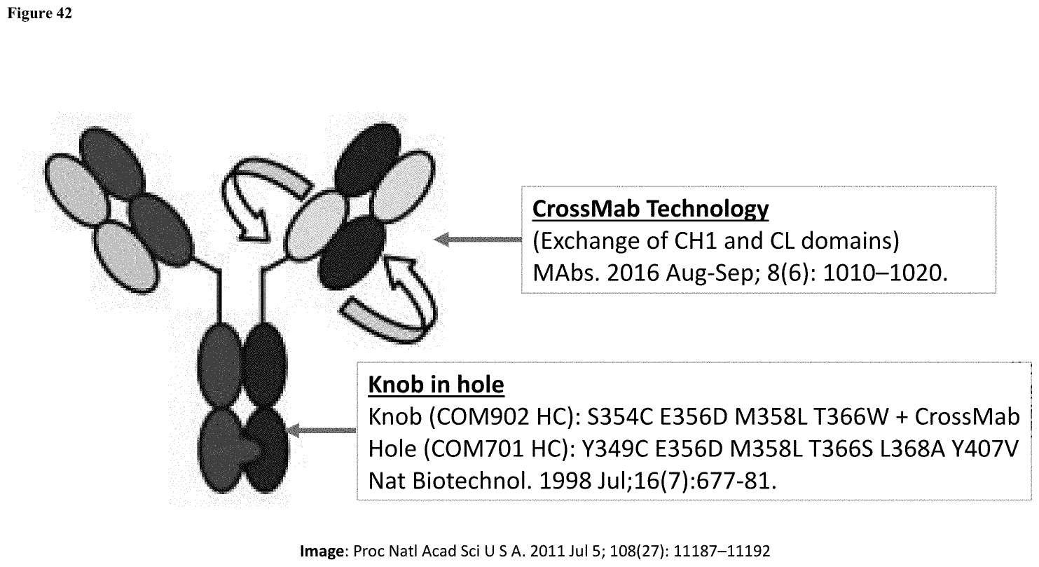

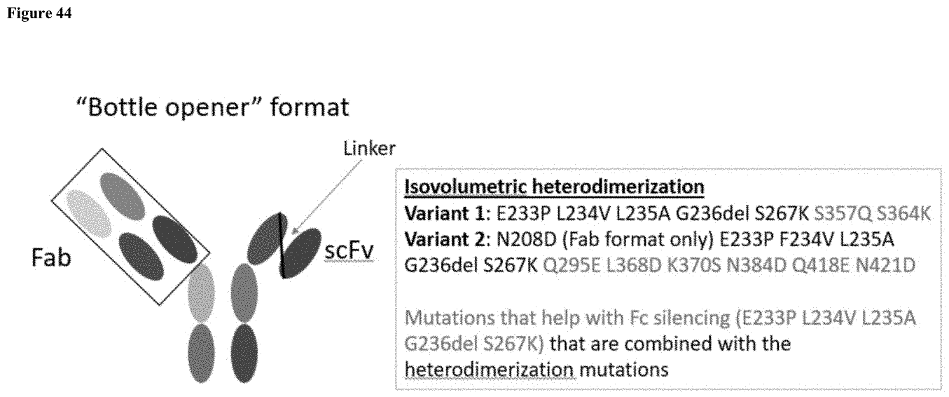

Representatives of multiple bispecific PVRIG/TIGIT dual inhibitor antibodies were created in three different formats using a combination of “knob into hole” (KIH) Fc engineering and CrossMab, KIH scFv-Fab (“bottle opener”), and “bottle opener” isovolumetric heterodimerization (“IH”) formats based sequences deposited at IMGT (http://www.imgt.org, positions based on Eu numbering; see also FIGS. 42-44). To create fully human bispecific antibodies in the KIH CrossMab format the CPA.9.086 variable heavy chain plus a two-amino acid linker of alanine and serine was fused to the human constant Lambda domain and the CH2 and CH3 domains of human IgG4 containing mutations S241P, S354C, E356D, M358L and T366W. The CPA.9.086 variable light chain domain plus a linker of two serines was fused to the CH1 of human IgG4. The CHA.7.518.1.H4(S241P) variable heavy chain domain was fused to the CH1 domain of human IgG4 and the CH2 and CH3 domains of human IgG4 containing mutations Y349C, E356D, M358L, T366S, L368A and Y407V. The CHA.7.518.1.H4(S241P) variable light chain domain was fused to the human constant Kappa domain. For the KIH “bottle opener” format, the same CHA.7.518.1 heavy chain and light chain constructs from above were used and paired with a single chain variable fragment (scFv) of CPA.9.086 fused to the hinge region of human IgG4 and also containing CH2 and CH3 with the “knob” mutations listed above. The order of heavy chain and light chain was varied in the single chain format (VH-VL or VL-VH) to assess effects on expression and stability. For the isovolumetric heterodimerization bottle opener format, several variable domain combinations were evaluated (CPA.9.086 scFv paired with CHA.7.518.1, CHA.7.518.1 scFv paired with CPA.9.086, CHA.9.547.18 scFv paired with CHA.7.518.4, and CHA.7.518.4 scFv paired with CHA.547.18) scFv containing constructs had the following substitutions in the CH2 and CH3 domains: F234V L235A G236del S267K S357Q S364K. Fab constructs contained the following substitutions: N208D E233P F234V L235A G236del S267K Q295E L368D K370S N384D Q418E N421D. Substitutions E233P F234V L235A G236del S267K and E233P F234V L235A G236del S267K K370S N384D Q418E N421D were incorporated to reduce Fc receptor binding.

Expression of CHA.7.518.1.H4(S241P)/CPA.9.086 Bispecific Antibody:

The DNA for each of these constructs was cloned into the pcDNA3.4 expression vector (ThermoFisher) to yield four separate vectors. To express bispecific antibodies, DNA for each construct was transfected into 3 ml logarithmically growing Expi293 cells (ThermoFisher) at a final total concentration of 1 ug/ml. Various ratios of heavy chains and light chains were analyzed for expression yield by LabChip (Perkin Elmer) capillary electrophoresis in both reducing and non-reducing conditions. This allows the relative amount of uncomplexed heavy chain and light chain to be qualitatively determined for multiple conditions. Once the optimal ratios were determined, larger scale productions were performed, and the expressed bispecific antibodies purified

Purification of CHA.7.518.1.H4(S241P)/CPA.9.086 Bispecific Antibody:

The anti-PVRIG/anti-TIGIT bispecific antibodies were purified using affinity purification and size-exclusion chromatography.

Affinity Purification with Protein A:

Protein A affinity chromatography (ProA) was performed on supernatant to purify bispecific antibody (BsAb) from cell culture supernatant. Supernatant was prepared for chromatography by the addition of 20 mL of 1M Sodium Phosphate pH 7.4 (Teknova) and 100 mL of 5M NaCl (Teknova) per 1000 mL of supernatant followed by 0.22 μm filtration. Separation was performed on an AKTA Pure (GE Healthcare) with a 5 mL HiTrap column of MabSelect SuRe (GE Healthcare) equilibrated with 20 mM Sodium Phosphate pH 7.4, 0.6M NaCl (Teknova). The supernatant was loaded at 1.25 mL/minute. Post load the column was washed with equilibration buffer for 10 CV at 5 mL/min. Elution was done in two steps. Step one was with 20 mM Sodium Citrate pH 3.6, 150 mM NaCl (Teknova) and step 2 was with 100 mM Glycine pH 2.7, 150 mM NaCl (Teknova). Both elutions were carried out over 4.3 CV at 5 mL/min. collecting 12 mL fractions into tube prefilled with 2.25 mL of 1 M Tris pH 7.5 (Teknova). Fractions were analyzed for protein concentration by absorbance at 280 nm using a NanoDrop (Thermo) and monomer content by analytical HPLC-SEC.

Separation of Monomer and High Molecular Weight (HMW) Components:

Preparative size exclusion chromatography (SEC) was performed on the Pro A purified bispecific antibody to separate monomer from HMW. BsAb was prepared for SEC chromatography by 0.22 μm filtration. Separation was performed on an AKTA Pure (GE Healthcare) running an isocratic gradient of PBS, pH 7.3 (Teknova). BsAb was loaded on to a Superdex 200 pg 26/600 column (GE Healthcare) at 2.6 mL/min. Elution was with 1 column volume (CV) of PBS at 2.6 mL/min. Fractions were collected after the void volume. HMW eluted ahead of the monomer as expected. Peak fractions were run on HPLC-SEC to determine monomer versus HMW percentage and concentration was determined by absorbance at 280 nm. Monomer and HMW containing fractions were separately pooled.

Sec-HPLC Analysis:

Analysis was performed using an Acquity UPLC (Waters) system and a Protein BEH SEC, 200A, 1.7 um, 4.6 mm×150 mm, 10K-450K (Waters) was used as the SEC column. The SEC separation was performed at ambient temperature isocratically using a mobile phase consisting of PBS, pH 7.4 (Teknova). Flow rate was 0.25 mL min-1. The relative amounts of BiSpAb monomer, aggregates and fragments were quantified by calculation of the peak areas detected by the ultraviolet (UV) detectors at 280 nm.

SPR Kinetic Assay of Human TIGIT and Human PVRIG Binding to the Bispecific Antibody:

All experiments were performed using a ProteOn XPR 36 instrument at 22° C. while samples were kept at 4° C. during the assay. First, high density capture surfaces were prepared with a goat anti-human Fc polyclonal antibody (Thermo Fisher) immobilized over all vertical capture lanes and horizontal interspots on a GLC chip (Bio Rad) using standard amine coupling. Typical immobilization levels for the polyclonal antibody for each lane were around 4600RU. Human TIGIT-HIS monomer was obtained from Sino Biological, while human PVRIG-HIS was prepared in-house. The bispecific antibody was diluted to ˜1 μg/ml in running buffer, which was 1×PBST with filtered BSA added to a final concentration of 100 μg/ml. For each “single-shot kinetics” cycle on the ProteOn instrument, a different antibody was captured over two unique vertical capture lanes for two minutes. After switching the buffer flow of the ProteOn to the horizontal direction, capture surfaces were stabilized for approximately 15-20 minutes. Over separate capture cycles, six concentrations of a 3-fold dilution series of human TIGIT (362 pM-88 nM) or human PVRIG (460 pM-112 nM) were injected for 2 minutes followed by 15 minutes of dissociation at a flow rate of 100 μl/min. Three replicates of each concentration series of antigen were injected over two independent surfaces of the captured bispecific antibody along with several cycles of buffer injections for double-referencing. Anti-human antibody surfaces were regenerated between each cycle with two 30-second pulses of 146 mM phosphoric acid. The sensorgrams of TIGIT and PVRIG injected over the captured bispecific antibody were processed using a ProteOn version of Scrubber and were fit to a 1:1 kinetic binding model, including a term for mass transport.

SPR Sandwich Assay of Human TIGIT and Human PVRIG Simultaneously Bound to the Bispecific Antibody:

Experiments were performed using a Biacore 3000 (GE Healthcare) at 22° C. Human TIGIT (Sino Biologicals) was covalently immobilized to all four flow channels of a CMS Biacore chip at RU levels that ranged from 800RU to 1000RU using standard amine coupling. The bispecific antibody was injected over one flow cell at a molecular concentration of 25 nM for three minutes at a flow rate of 25 μl/min. Human PVRIG was then simultaneously injected at a concentration of 116 nM for three minutes at a flow rate of 25 μl/min over both the TIGIT flow cell complexed with the bispecific antibody and over a control TIGIT surface having no bound bispecific antibody. The control TIGIT surface was reference subtracted using Scrubber 2.0 software to process the resulting sensorgrams.

Dual Engagement ELISA:

To prepare ELISA plates for analysis of bispecific antibody binding, plates were coated overnight at 4° C. with 100 ul of 1 ug/mL PVRIG-hFc. in PBS. The following day, the coating solution was discarded, and the plate blocked with 250 μL of 2% BSA, 0.1% Tween 20 for 2 hours. The wells were washed 3× with 300 μL of 1×PBS pH 7.4, 0.05% Tween20 (wash buffer). Samples were serially diluted in 3-fold increments in 1×PBS pH7.4, 0.1% Tween20, 0.2% BSA (binding buffer) from 1 μg/mL to 1.4 pg/mL and incubated at room temperature for 1 hour with shaking at 300 RPM. As controls, CHA.7.518.1.H4(S241P), a PVRIG-specific antibody, and CPA.9.086, a TIGIT-specific antibody, were similarly prepared and evaluated as the bispecific samples. Wells were washed as above and the ability to bind TIGIT was assessed by the addition of 100 μL of 1 μg/mL TIGIT-His (Sino Biological cat #10917-H08H). For assessing the control antibody binding, 100 μL of 1 μg/mL PVRIG-His (Compugen, lot 20170623) was added to the CHA.7.518.1.H4(S241P) wells and 100 ul of 1 μg/mL TIGIT-His added to the CPA.9.086 wells. Incubation with soluble ligands was for 1 hour at room temperature with shaking at 300 RPM. Wells were washed as above, and then soluble ligand binding was detected by the addition of 100 ul of 1 ug/ml anti-His-Tag antibody HRP conjugate (R&D Systems, Catalog # MAB050H, clone# AD1.1.10 in binding buffer and incubated at room temperature with shaking at 300 RPM for 1 hour. Wells were washed 3× with wash buffer as above and 100 μL of room temperature Ultra-TMB substrate (Moss Inc.) added. Development was for 7 minutes and the reaction stopped by the addition of 100 μL 2N H2504. The plate was read on Molecular Devices SpectraMax 340PC-384 plate reader set at wavelength of 450 nM.

To assess TIGIT-capture and soluble PVRIG binding of the bispecific antibodies, as similar assay to that used above was performed. In this case, 100 μL/well of 1 μg/ml TIGIT-Fc fusion protein in 1×PBS pH7.4 was coated overnight at 4° C. The coating solution was discarded, and the wells blocked with 1×PBS pH 7.4, 2% BSA and 0.1% Tween 20 for 2 hours at room temperature. Bispecific and monospecific controls used above were serially diluted in the same range as for the PVRIG capture experiment. The remainder of the experiment was the same as above with the exception that PVRIG-His was used for detection of all samples except CPA.9.086 for which TIGIT-His was used.

Human CMV-Specific CD8⁺ T Cell Expansion:

Human CMV-reactive peripheral blood mononuclear cells (PBMCs) (CTL) were thawed, resuspended at 2×10⁶ cells/ml, and stimulated with 1 μg/ml of the CMV pp65 peptide (Anaspec) in complete RPMI medium supplemented with 2 ng/ml recombinant human IL-2 (R&D systems) and 10 ng/ml recombinant human IL-7 (R&D systems) at 37° C. with 5% CO2. After 6 days, cells were split 1:2 and rested with low dose human IL-2 (100 IU/ml). At day eight, cells were harvested, and re-plated in low dose IL-2 (100 U/ml) at 2 million/ml in complete RPMI media for two days. At day eleven, cells are phenotyped for CD8⁺ T cell purity and CMV pp65₍₄₉₅₋₅₀₃₎ reactivity.

Cells were stained with anti-CD3 (clone: OKT3)-allophycocyanin seven (APC-Cy7; Biolegend), anti-CD8 (clone: H1T8a)-Alexa Fluor (AF) 488 (Biolegend), a combined cocktail of anti-CD14 (clone: HCD14)-peridinin chlorophyll protein (PerCP-Cy5.5), anti-CD19 (clone:HIBCD14) PerCP-Cy5.5, anti-CD56 (clone:HCD56)-PerCP-Cy5.5(Biolegend), anti-TIGIT (clone: MB SA43)-allophycocyanin (APC; e-Bioscience) or IgG4 (Compugen)-isotype control (APC:Biolegend), CHA.7.518.1.H4(S241P)-AF-647 (Compugen) or IgG4-AF647 isotype control (Compugen), and anti-PD-1 (clone: EH12.2H7)-Brilliant Violet 421 (BV421:Biolegend) or IgG1 (clone: MOPC21) BV421 (Biolegend). To assess the frequency of tetramer-reactive CD8⁺ T cells, PBMCs were stained with iTAg Tetramer-HLA-A*02:01 CMV pp65₍₄₉₅₋₅₀₃₎ (NLVPMVATV)-phycoerythrin (MBL-BION) for 30 min at room temperature. Cells were washed with PBS/1% BSA/0.01% sodium azide buffer, data were acquired using a Fortessa flow cytometer (BD Biosciences) and analyzed using FlowJo (Treestar).

Human CMV-Specific CD8⁺ T cell Co-Culture Assay with pp65-Expressing Melanoma Cell Lines:

An in vitro co-culture assay with human CMV-specific CD8⁺ T cells was utilized to assess the effect of monoclonal antibodies (mAbs) to PVRIG or TIGIT and a bispecific antibody (BsAb) to PVRIG and TIGIT on antigen-specific cytokine secretion. The target cells used in the co-culture assay was a modified Mel-624 cell line (ATCC), Mel-624 pp65. This cell line was generated by ectopically expressing the CMV protein, pp65. This results in HLA-A2 presentation of the peptides derived from pp65 without the need to add exogenous pp65 peptides. A dose-dependent titration of inhibitory receptor blockade on CMV pp65 reactive CD8 T cells in co-culture with Mel-624 pp65 cancer cell line was performed. A 10 point, 4-fold dilution series of antibody, starting at 132 nM and ending at 0.001 nM was co-cultured with Me1624 pp65 cells and CMV⁺CD8⁺ T cells in a 5:1 ratio of target cells (75,000) to T cells (15,000). T cells, tumor cell lines, and antibodies were added together in a 96-well U-bottom plate (Costar), and incubated for 18 hours at 37° C. The following antibodies were tested: anti-PVRIG (CHA.7.518.1.H4(S241P)), anti-TIGIT (CPA.9.086), a combination of CHA.7.518.1.H4(S241P) and CPA.9.086, bispecific antibodies (CHA.7.518.1.H4(S241P) or CHA.7.518.4/CPA.9.086 or CHA.9.547.18 BsAb) that targets both PVRIG and TIGIT, and a human IgG4 isotype control. After the 18 hours incubation period, the amount of human interferon gamma (IFN-γ) in the co-culture supernatant was measured by flow cytometry using a cytometric bead assay (BD). Data were analyzed by non-linear regression and fit to either a “One site specific binding” or “One site—Total and nonspecific binding” model using GraphPad Prism.

Differential Scanning Fluorimetry:

Melting temperature provides a measure of antibody stability. The bispecific and control monospecific antibodies were analyzed for melting temperature denaturation and binding of the hydrophobic dye SYPRO Orange (Thermo). Antibodies were diluted to an appropriate concentration in the desired buffer with SYPRO Orange. A StepOne Plus RT-PCR instrument (Applied Biosystems) was used to perform a controlled melt (25 C-95 C, 1 degree/minute ramp) and detect fluorescence of SYPRO Orange at 1-minute intervals. The antibodies were analyzed at pH6.0 and pH7.4 to ensure that the effects obaserved were not due to the formulation.

LC-MS Analysis:

Mass spectrometry was performed by LakePharma Inc. in Belmont, Calif., USA. After enzymatic deglycosylation, samples were analyzed on an Agilent Q Exactive Orbitrap (Thermo Fisher Scientific) coupled to a capillary UHPLC system. Intact analysis was performed under nonreduced and reduced conditions for protein characterization and Waters MassLynx software used to analyze the results.

Freeze-Thaw Stability Analysis:

Samples were prepared by adjusting the concentration to 1.0 mg/mL in 1×PBS, pH7.4 by dilution or using a spin concentrator. 100 ul was put into 0.2 ml snapstrip PCR tubes and frozen at −80° C. for at least 24 hours, then thawed at room temperature. This cycle was repeated twice for 3 cycles of freezing and thawing. 20 ul of each sample was analyzed by SEC-UPLC for monomer, high molecular weight (HMW) and low molecular weight (LMW) species. The samples were also run in CE-SDS (reduced and non-reduced, Lab Chip GXII, Perkin Elmer) and concentration assessed after centrifugation to remove any precipitated protein.

Low pH Hold:

Samples were prepared by adjusting the concentration to 1.0 mg/mL in 1×PBS, pH7.4 by dilution or using a spin concentrator. The pH of each sample was adjusted to pH3.0 by addition of 0.1 M Glycine pH3.2 and incubated at room temperature for 0, 1, 2, 4, and 24 hours. The samples were neutralized with 1 M Tris pH7.5 and analyzed by CE-SDS (reduced and non-reduced, Lab Chip GXII, Perkin Elmer), SEC-UPLC and absorbance at 280 nm by NanoDrop after centrifugation to remove any precipitated protein.

### Results

Sequence of Bispecific Antibodies:

The anti-PVRIG/anti-TIGIT bispecific antibodies were created using two different Fc heterodimerization formats and two variable domain display formats. The sequence of each fragment is shown in FIGS. 26A-26B, 40A-40T and 41A-41T.

SPR Kinetic Assay:

FIG. 27 shows the sensorgram data of (A) TIGIT and (B) PVRIG binding to the captured bispecific antibody over two independent surfaces and the global fits of the data to a simple 1:1 kinetic model. Because there was not enough dissociation signal decay for the kinetic model to adequately estimate the off-rate for the TIGIT-bispecific antibody interaction, the k_{d} value was arbitrarily held constant at 1.0e⁻⁵ sec⁻¹ in order to fit the data. The affinity and rate constants from the TIGIT-bispecific antibody interaction in FIG. 27 should therefore only be considered to be rough estimates. The binding constants of the TIGIT-Bispecific antibody interaction as measured using SPR with the k_{d} held constant at 1.0e⁻⁵ sec⁻⁴ were kₐ=3.9e⁶ M⁻¹ sec⁻¹ and K_{D}=2.6 pM. The binding constants for the PVRIG-Bispecific antibody interaction were estimated to be kₐ=1.3e⁶ M⁻¹ sec⁻¹, k_{d}=2.4e⁻⁴ sec⁻¹, and K_{D}=187 pM.

SPR Sandwich Assay:

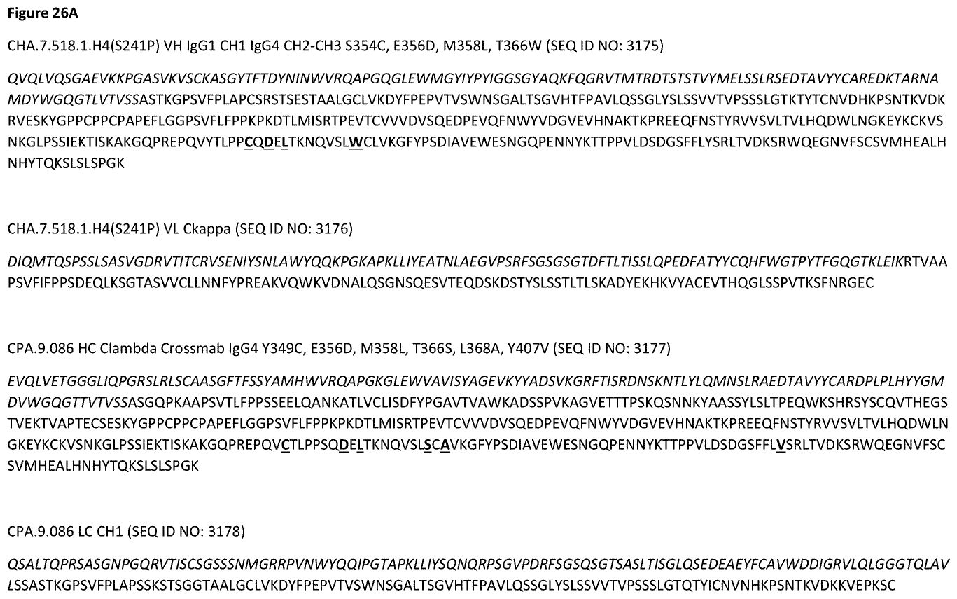

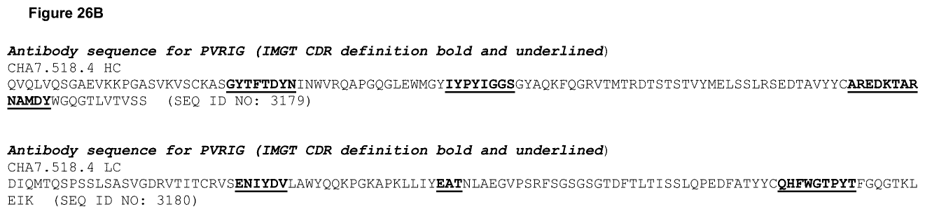

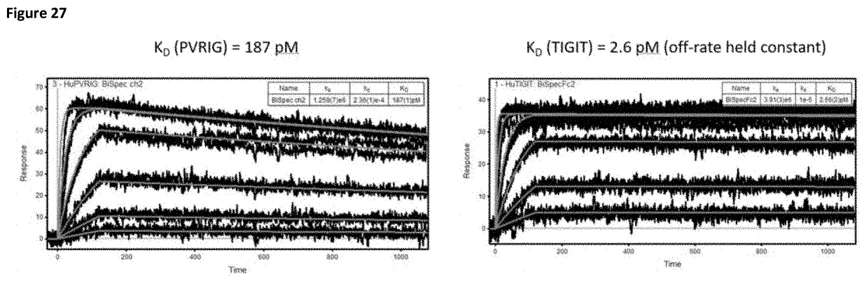

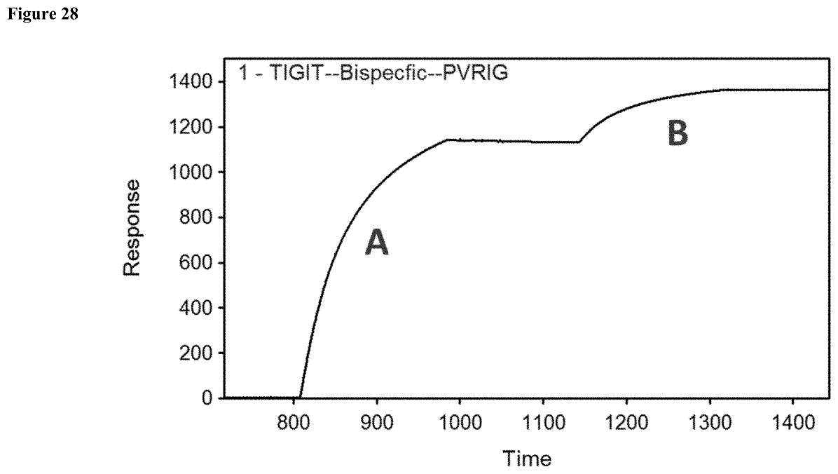

FIG. 28 shows the sensorgram of successive injections of the bispecific antibody (A) injected over the Biacore chip having immobilized human TIGIT followed by an injection of human PVRIG (B), therefore showing the ability of the bispecific antibody to simultaneously bind both human TIGIT and human PVRIG. The response level of the bispecific antibody binding to the TIGIT surface was ˜1100RU. The response level of human PVRIG binding to the bispecific antibody pre-complexed to the TIGIT surface was ˜230RU.

CMVpp65 Reactive T Cells Express PVRIG, TIGIT, and PD-1:

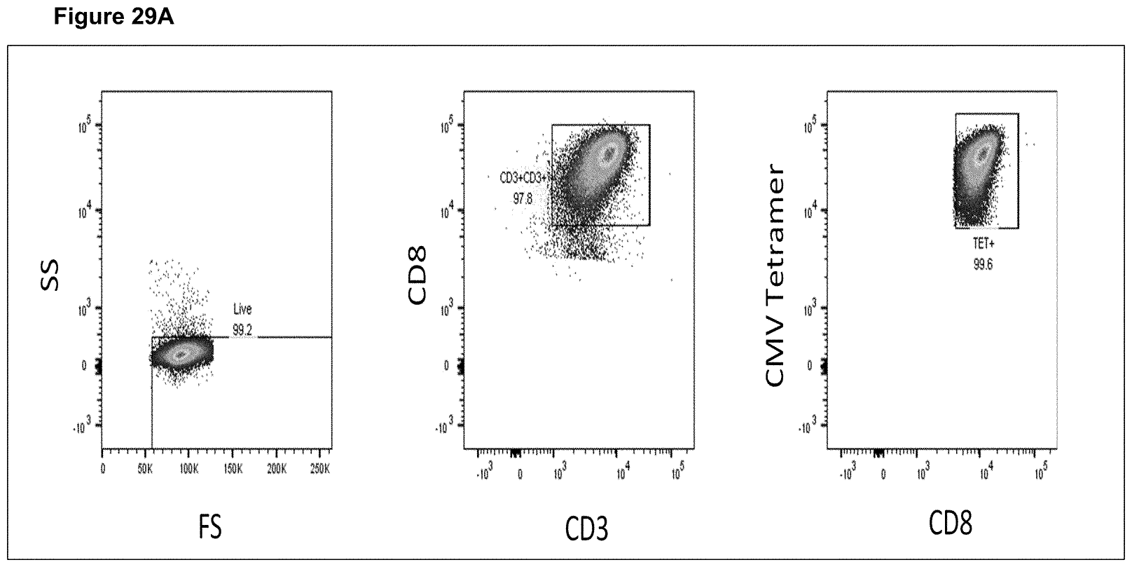

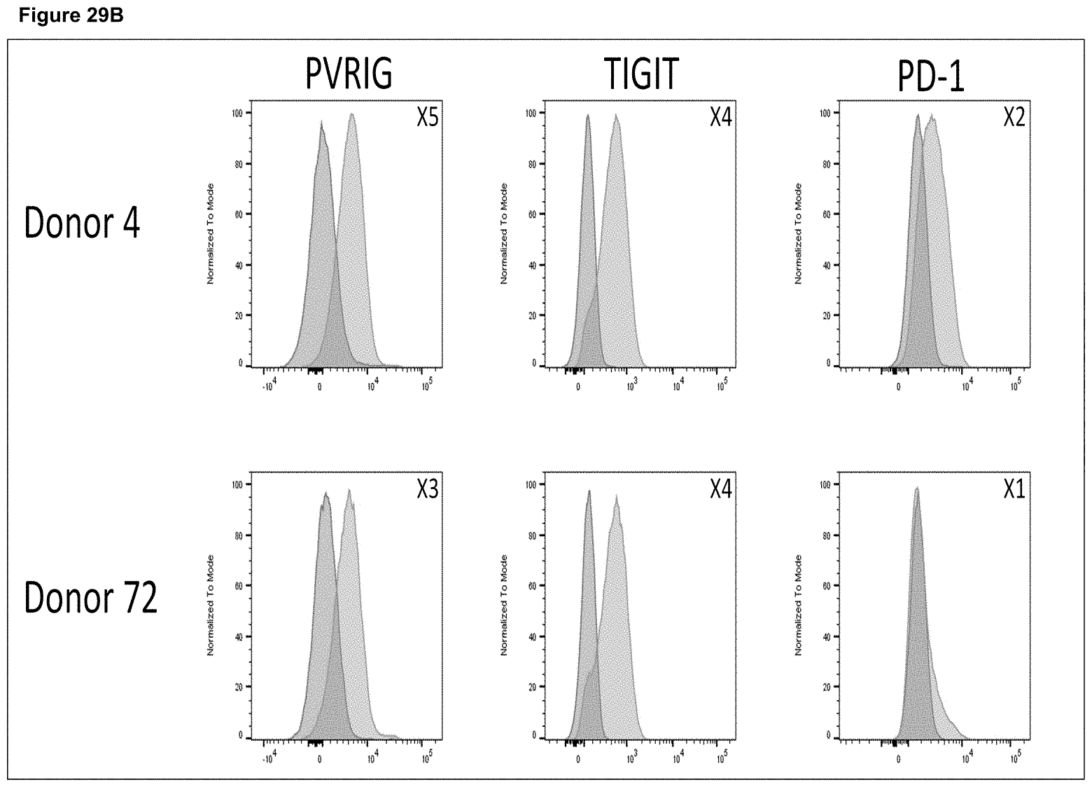

CD8⁺ T cells specific to a CMV protein, pp65, have been well characterized and these CMV specific T cells can be used to study the role of modulatory receptors on T cells. Stimulation of HLA-A2⁺ donor PBMCs using CMV pp65 peptide, IL-2 and IL-7 resulted in a strong expansion of CMV pp65-specific T cells to purities ranging from 90-95% as determined by tetramer staining. FIG. 29A shows the percentage of CMV pp65 specific T cells from two donors after expansion. The surface expression of PVRIG, TIGIT and PD-1 on T cells was assessed from these donors. On day eleven of activation, CMV pp65 specific T cells expressed PVRIG gMFI ratio (MFIr, defined as the MFI of target antibody divided by MFI of control antibody): 3-5-fold, TIGIT MFIr: 4-fold), and PD-1 MFIr: 1-2-fold (FIG. 29B).

Anti-Human PVRIG and TIGIT Antibodies Increase IFN-γ Secretion Either Alone or in Combination from Human CMV-Specific T Cells:

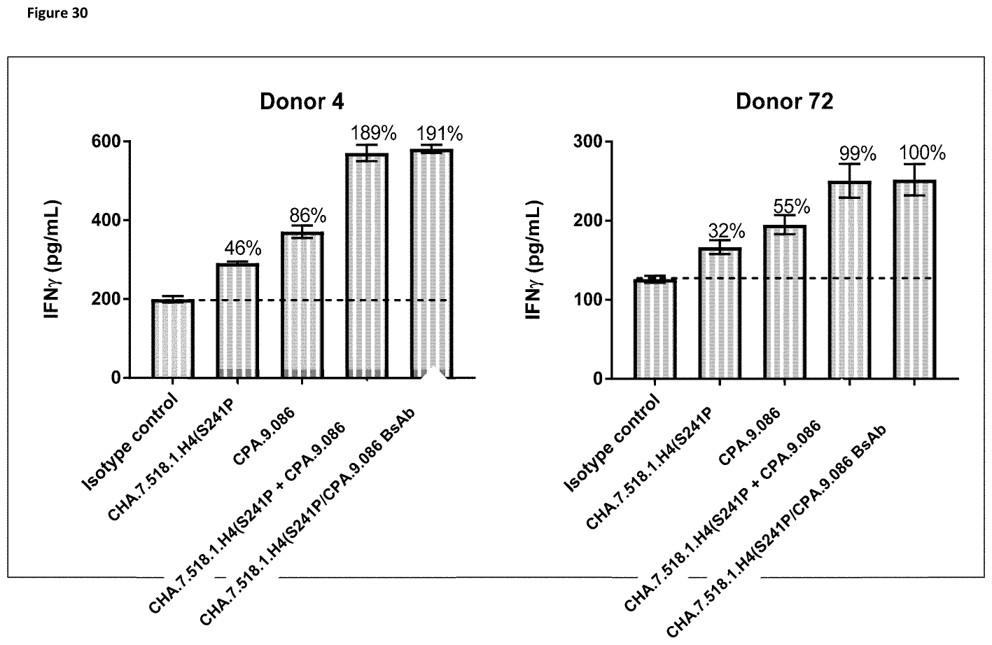

With the rationale that the induction of TIGIT and PVRIG expression on CD8⁺ T cells correlates with T cell dysfunction, we aimed to evaluate the effects of PVRIG and TIGIT blockade on the capacity for pro-inflammatory cytokine production. CMV reactive T cells from 2 donors (Donor 4 and Donor 72) were co-cultured with Mel-624 (PVR⁺PVRL2⁺) pp65 cells. The activity of CHA.7.518.1.H4(S241P), CPA.9.086, CHA.7.518.1.H4(S241P) and CPA.9.086, and CHA.7.518.1.H4(S241P)/CPA.9.086 BsAb on CMV⁺CD8⁺ T cells was assessed. We observed that CHA.7.518.1.H4(S241P) increased IFN-γ production (32-46%) as compared with IgG control antibody. Addition of CPA.9.086 antibody resulted in a further increase in IFN-γ (55-86%). When combined, CHA.7.518.1.H4(S241P) and CPA.9.086 treatment synergistically or in some cases, additively increased cytokine production of CD8⁺ T cells compared with CHA.7.518.1.H4(S241P) or CPA.9.086 single blockade (99-189%). The CHA.7.518.1.H4(S241P)/CPA.9.086 BsAb exhibited the same functional effect as the combination CHA.7.518.1.H4(S241P) and CPA.9.086 2 mAbs (100-191%). The percent increase of IFN-γ secretion in each antibody over respective isotype control antibodies is shown in FIG. 30.

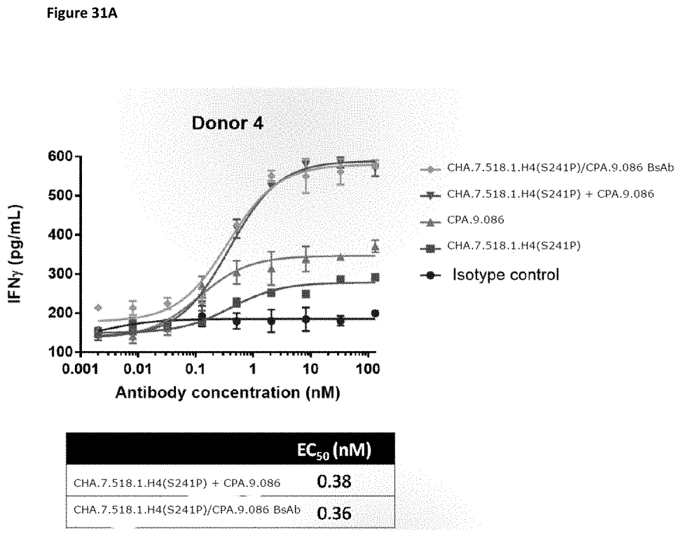

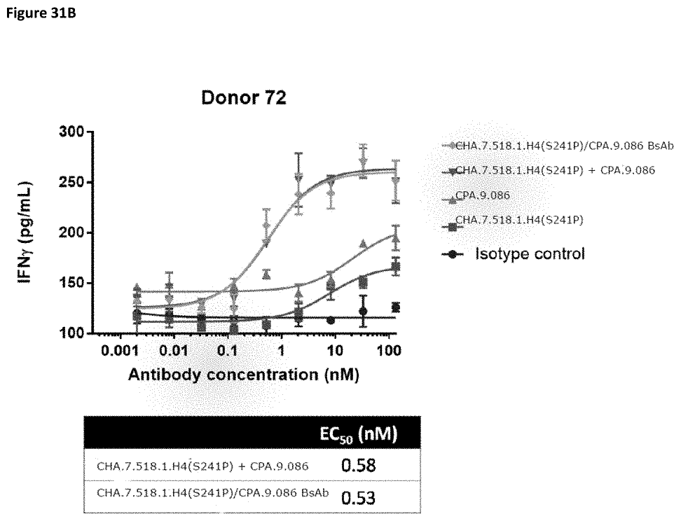

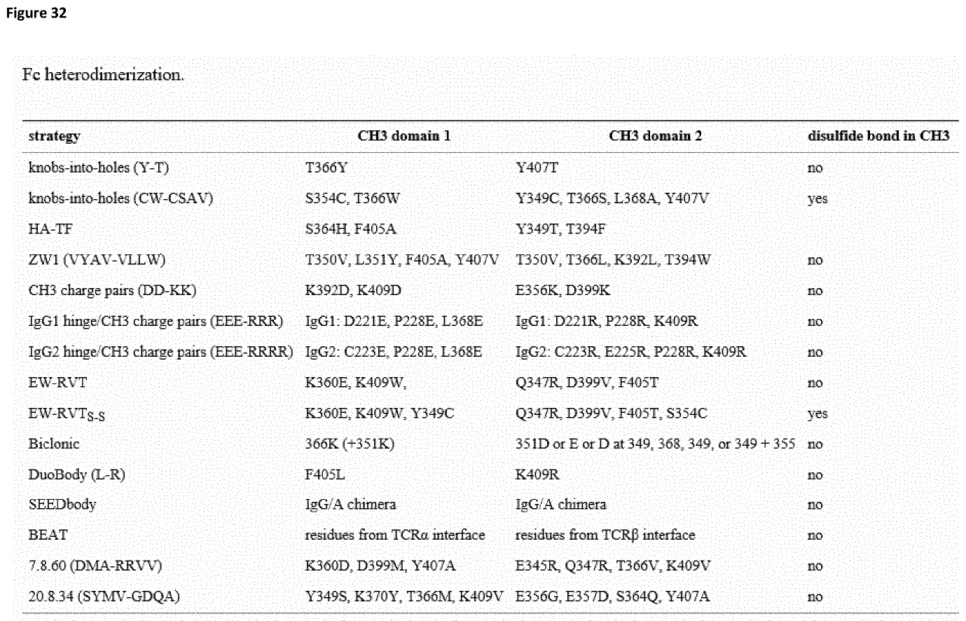

It was also observed that the CHA.7.518.1.H4(S241P)/CPA.9.086 BsAb displayed a dose dependent increase in IFN-γ production by CMV pp65 specific T cells which is comparable to the combination of CHA.7.518.1.H4(S241P) and CPA.9.086 mAbs (FIG. 31A-31B). In both donors 4 and 72, the average±SEM IFN-γ production EC₅₀ values for the CHA.7.518.1.H4(S241P)/CPA.9.086 BsAb and the combination of CHA.7.518.1.H4(S241P) and CPA.9.086 were similar and calculated to be in the low single digit nM range. In donor 4, the EC₅₀ was calculated to be 0.38 nM for the combination of CHA.7.518.1.H4(S241P) and CPA.9.086, whilst the CHA.7.518.1.H4(S241P)/CPA.9.086 BsAb EC₅₀ was calculated to be 0.36 nM. In donor 72, the EC₅₀ was calculated to be 0.58 nM for the combination of CHA.7.518.1.H4(S241P) and CPA.9.086, whilst the CHA.7.518.1.H4(S241P)/CPA.9.086BsAb EC₅₀ was calculated to be 0.53 nM (FIG. 31A-31B).

Dual Engagement ELISA:

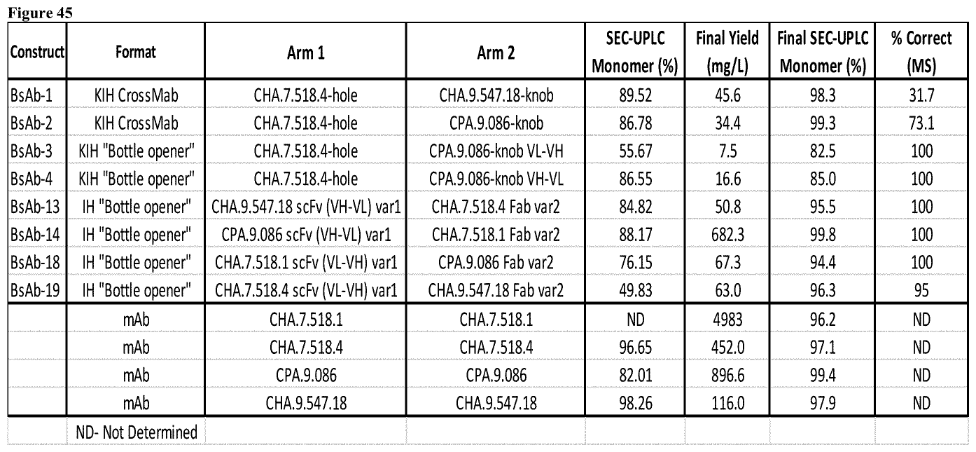

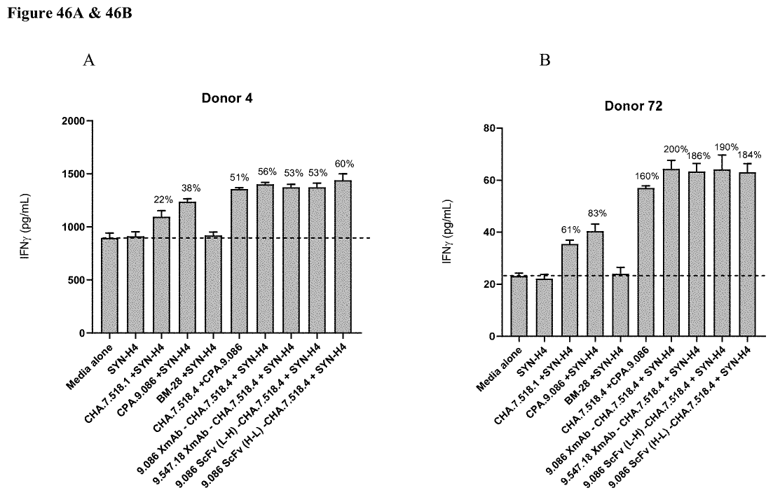

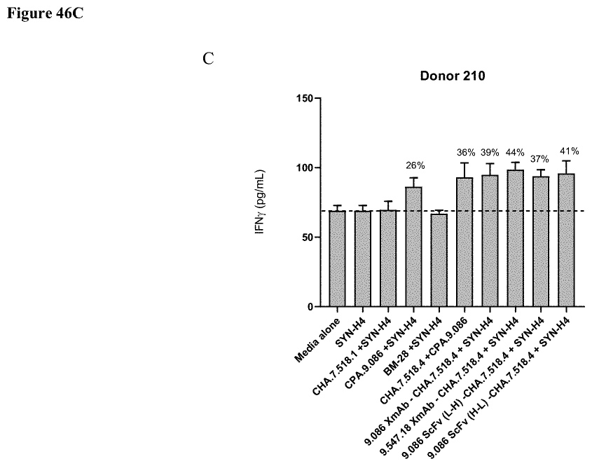

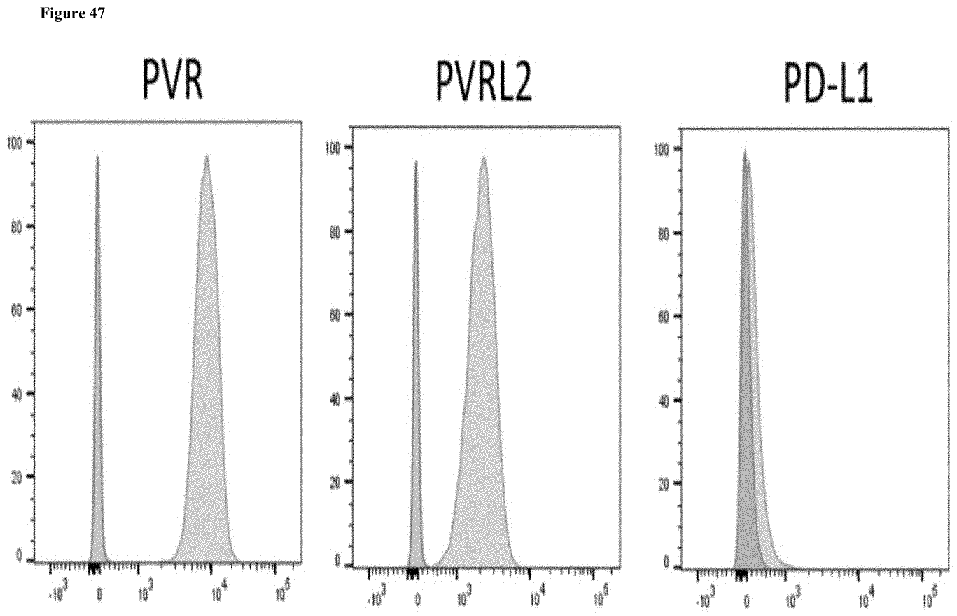

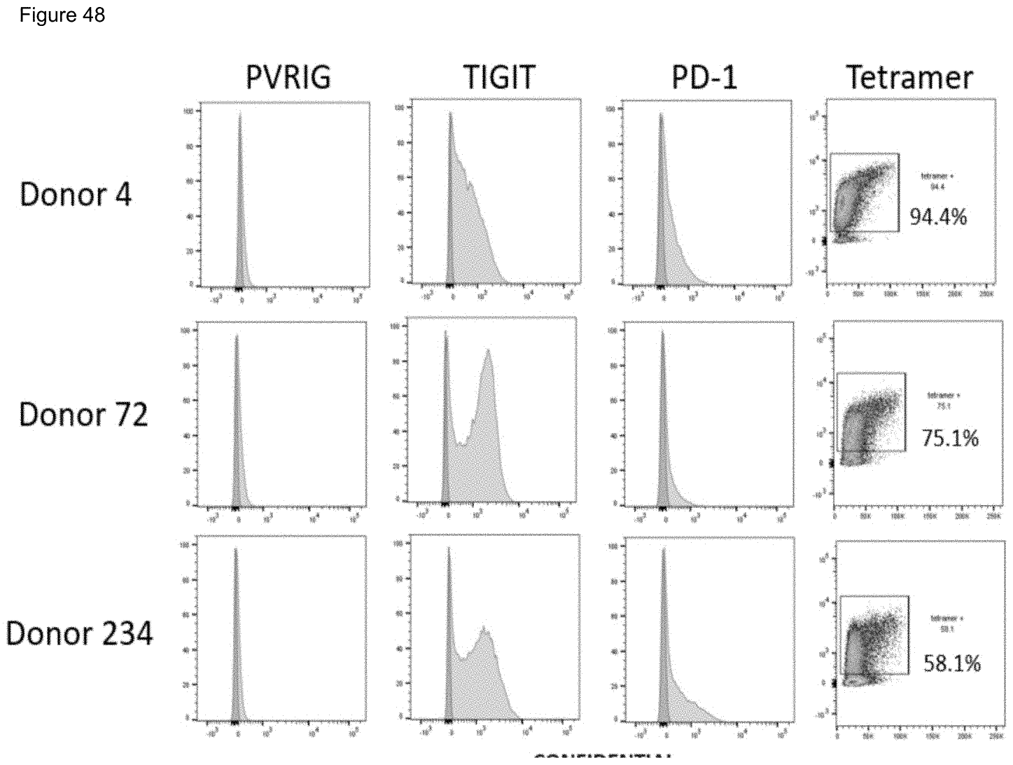

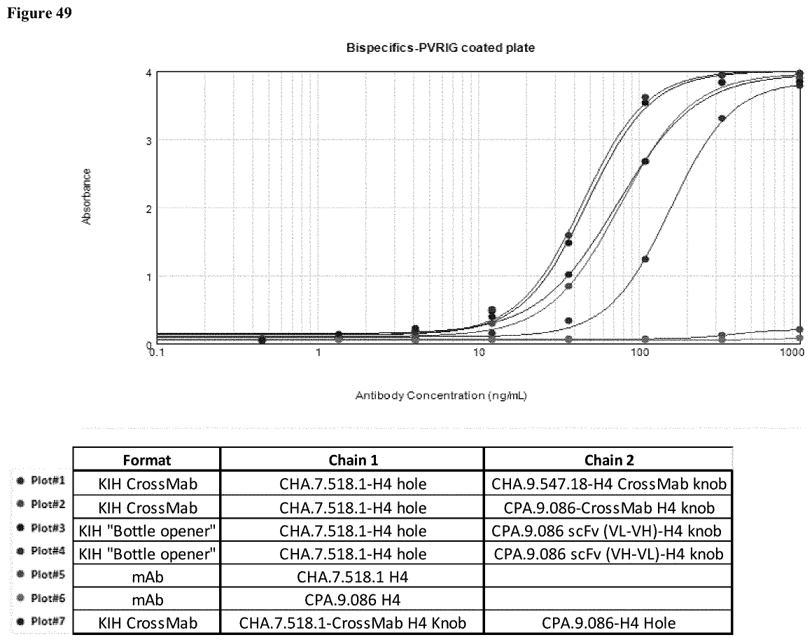

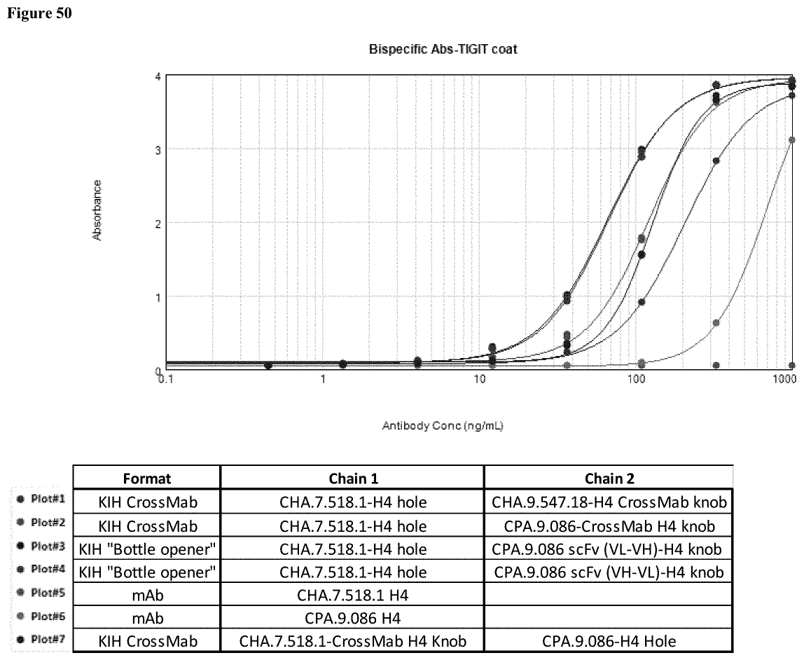

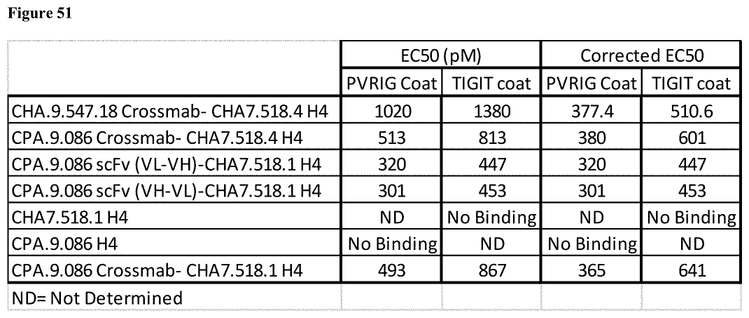

Bispecific and monospecific antibodies were assessed for the ability to bind coated antigen and then bind a second, soluble ligand simultaneously. FIG. 49 shows the binding of 3-fold serial dilutions of bispecific and monospecific antibodies to PVRIG coated plates with detection through the binding of biotinylated TIGIT-His protein and Streptavidin-HRP. Similarly, FIG. 50 shows binding of 3-fold serial dilutions of bispecific and monospecific antibodies to recombinant TIGIT-His coated plates and detection through binding of biotinylated PVRIG and Streptavidin HRP. All bispecific formats were able to engage both ligands simultaneously. Apparent shifts in affinity was due to the relative amount of correctly assembled antibody in solution. FIG. 51 shows the EC₅₀s calculated using SoftMax Pro software (Molecular Devices) and corrected for correctly assembled bispecific antibody as determined by LC-MS and shown in FIG. 45.

Differential Scanning Fluorimetry:

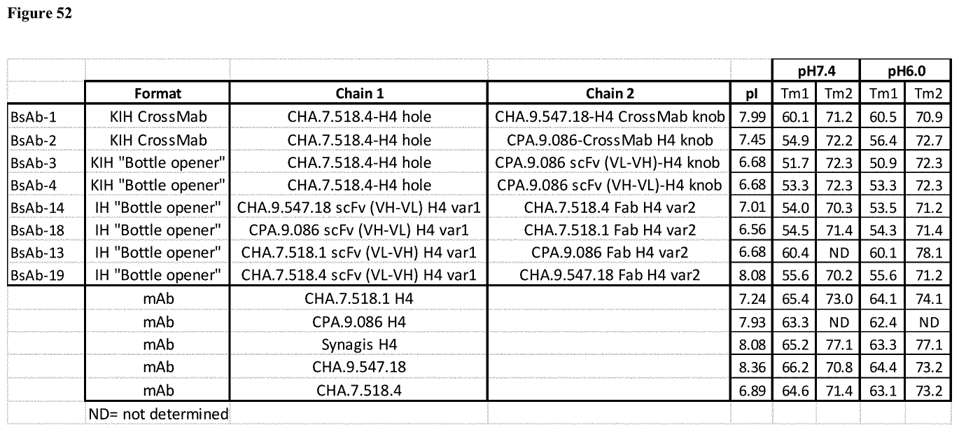

The bispecific and monospecific antibodies were analyzed for melting temperature denaturation and binding of the hydrophobic dye SYPRO Orange (Thermo). The antibodies were analyzed at pH6.0 and pH7.4 to ensure that the effects observed were not due to the formulation. Tm1 which reflects the dissolving of the CH2 domain was in the range of 51.7 C to 60.4 C at pH7.4 and 50.9 C to 60.5 C at pH6.0 for all the bispecific antibodies (FIG. 52). The monospecific antibodies all demonstrated a higher Tm1, 63.3 C to 66.2 C at pH7.4 and 62.4 C to 64.1 C at pH6.0. Tm2 for all the antibodies (CH3 and Fab melting) were >70 C (range=70.2 C-77.1 C at pH7.4 and 70.9 C to 77.1 C at pH6.0).

LC-MS Analysis:

Deglycosyated intact mass spectrometry was performed on the bispecific antibodies of all three formats. Heterodimerization utilizing knobs into holes Fc and CrossMab strategies resulted in 32% and 73% correctly assembled bispecific antibody, while the two “bottle opener” formats containing a scFv containing arm and an intact antibody arm through different Fc heterodimerization approaches (knobs into holes and isovolumetric heterodimerization, respectively) resulted in >95% correctly assembled bispecific antibody (FIG. 45).

Low pH Hold:

Bispecific and monospecific control antibodies were incubated at pH3 for 0, 1, 2, 4, and 24 hours and assessed for the formation of aggregates or lower molecular weight products by CE-SDS using a LabChip GXII (Perkin Elmer) and SEC-UPLC as observed by changes to the % monomer content from T=0. All bispecific and monospecific antibodies were stable to this stress, showing at most a 6% change in monomer content over the 24 hour time course.

Freeze-Thaw Stability Analysis:

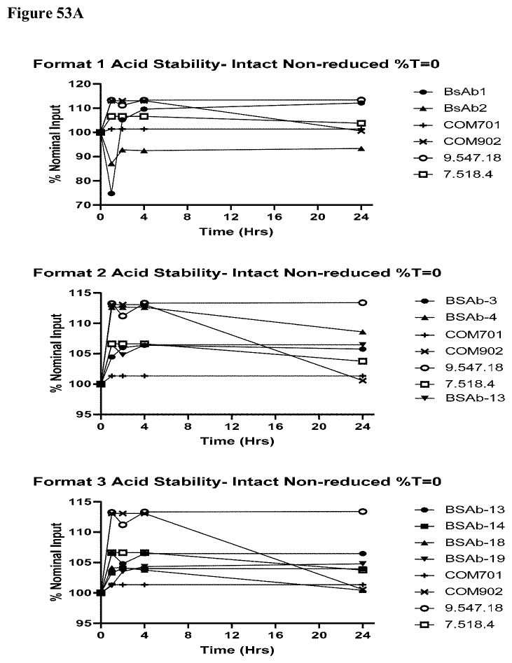

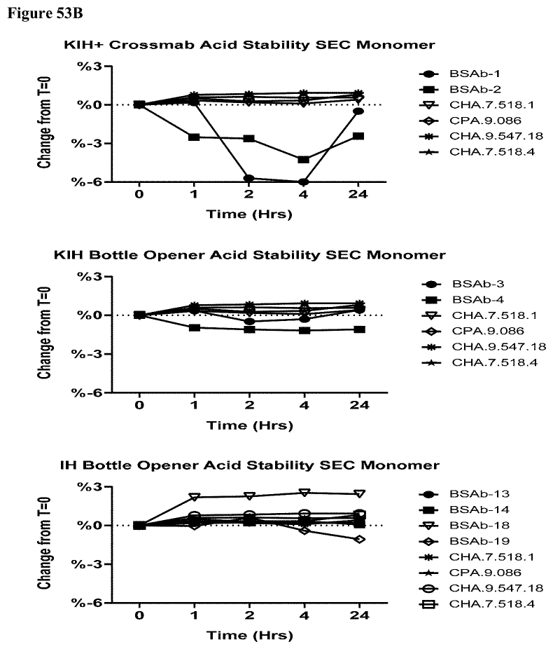

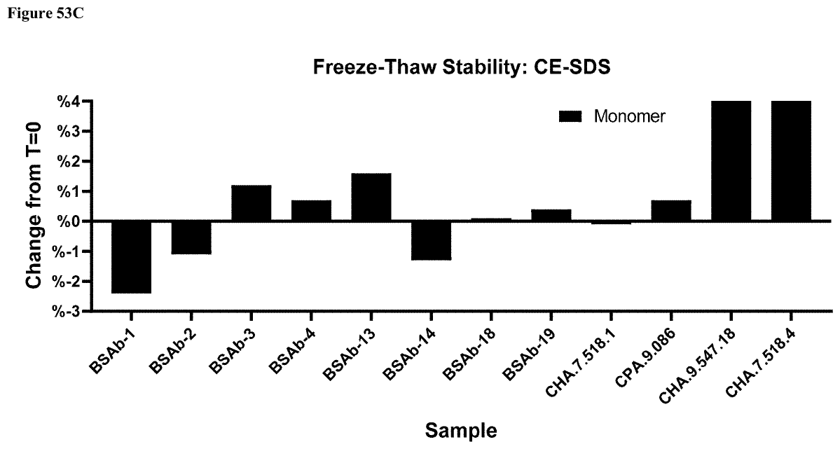

Bispecific and control monospecific antibodies utilizing the various light chain constraining and heterodimerization approaches were assessed for their ability to withstand serial freeze thaw cycles. After three cycles of freezing and thawing, the antibodies were analyzed by SEC-UPLC for changes in the Low Molecular Weight (LMW), Monomer, and High Molecular Weight species present in solution. Positive values represent an increase in the relative proportion of the species reported after the freeze/thaw cycles. Bispecific antibody BsAb-14 was the only sample that showed a >5% decrease in monomer content from T=0 (FIG. 53A-53D). All other formats were stable after this stress. CMV assay dose response

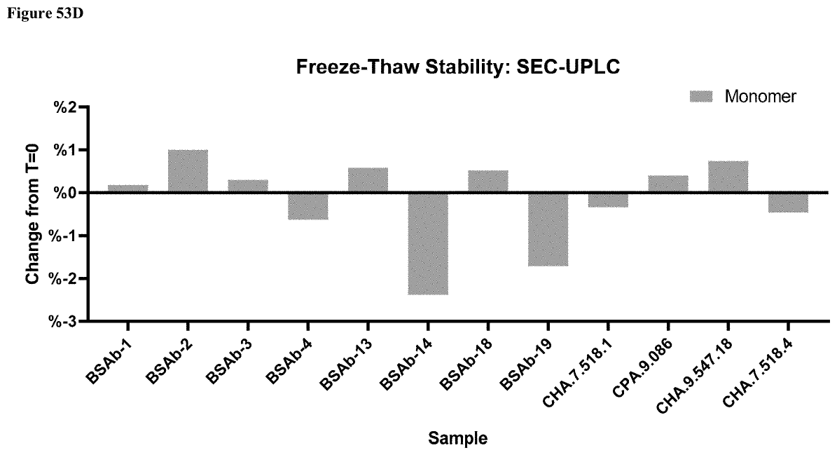

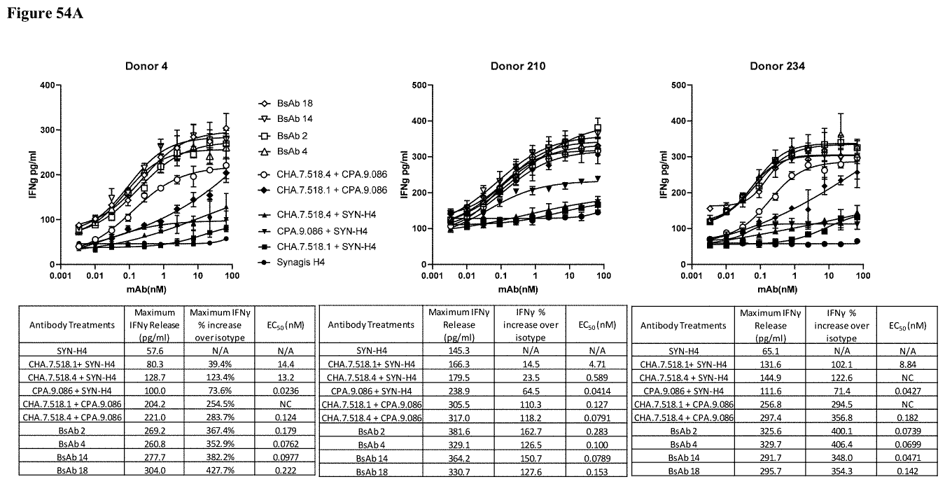

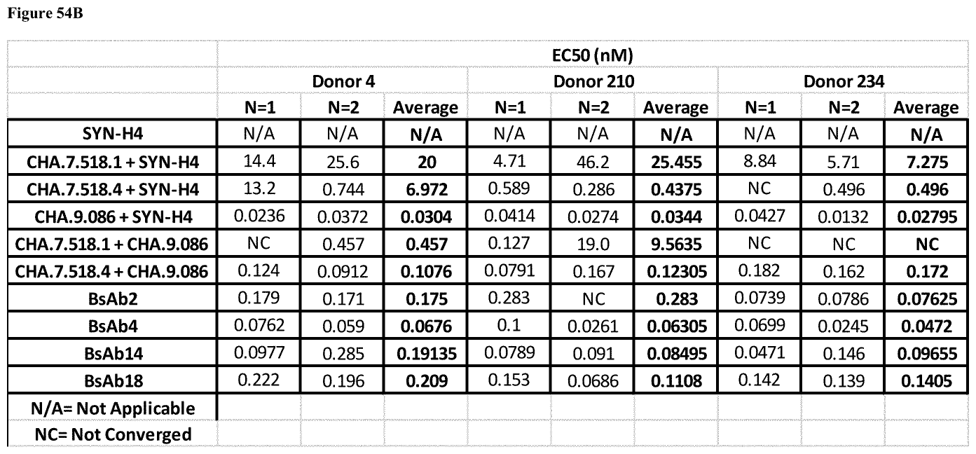

Bispecific, monospecific and combinations of monospecific antibodies were tested in the CMV assay described for FIG. 30 in a dose response. Serial three-fold dilutions of each antibody or mixture were performed to provide a range of antibody concentrations from 66 nM to 0.003 nM. Monospecific antibodies were mixed 1:1 with a non-specific control antibody prior to serial dilution. The bispecific antibodies tested were BsAb 2 (KIH+CrossMab), BsAb 4 (KIH bottle opener), BsAb 14, and BsAb 18 (both IH bottle opener). Combinations of CHA.7.518.1 and CHA.7.518.4 with CPA.9.086 were also assessed. The bispecific antibodies all performed at least equally to the combination of antibodies across all the formats tested and were superior to the monospecific antibodies in all three donor T-cells tested in two independent assays (FIG. 54A-54B).

### SUMMARY AND CONCLUSIONS

Addition of CHA.7.518.1.H4(S241P) and CPA.9.086, either alone or in combination, and the CHA.7.518.1.H4(S241P)/CPA.9.086 BsAb to the Mel-624 pp65 assay induced a dose dependent increase in IFN-γ secretion compared with IgG control antibody. When combined, CHA.7.518.1.H4(S241P) and CPA.9.086 synergistically or in the case of donor 72, additively increased cytokine production of CD8⁺ T cells compared to single blockade alone. The CHA.7.518.1.H4(S241P)/CPA.9.086 BsAb exhibited the same functional effect as the combination of CHA.7.518.1.H4(S241P)/CPA.9.086 mAbs with EC₅₀ values for both donors calculated to be in the low single digit nM range. Taken together, these data demonstrate that CHA.7.518.1.H4(S241P) and CPA.9.086 antibodies, either alone or in combination, or in a bispecific antibody format can enhance T cell effector function.

### Example 2: ELISA Assay

### PVRIG Coated Plate

To prepare ELISA plates for analysis of bispecific antibody binding, plates were coated overnight at 4° C. with 100 ul of 1 ug/mL PVRIG-hFc. in PBS. The following day, the coating solution was discarded, and the plate blocked with 250 μL of 2% BSA, 0.1% Tween 20 for 2 hours. The wells were washed 3× with 300 μL of 1×PBS pH 7.4, 0.05% Tween20 (wash buffer). Samples were serially diluted in 3-fold increments in 1×PBS pH7.4, 0.1% Tween20, 0.2% BSA (binding buffer) from 1 μg/mL to 1.4 pg/mL and incubated at room temperature for 1 hour with shaking at 300 RPM. As controls, CHA.7.518.1 H4, a PVRIG-specific antibody, and CPA.9.086 H4, a TIGIT-specific antibody, were similarly prepared and evaluated as the bispecific samples. Wells were washed as above and the ability to bind TIGIT was assessed by the addition of 100 μL of 1 μg/mL TIGIT-His (Sino Biological cat #10917-H08H). For assessing the control antibody binding, 100 μL of 1 μg/mL PVRIG-His (Compugen, lot 20170623) was added to the CHA.7.518.1 H4 wells and 100 ul of 1 μg/mL TIGIT-His added to the CPA.9.086 H4 wells. Incubation with soluble ligands was for 1 hour at room temperature with shaking at 300 RPM. Wells were washed as above, and then soluble ligand binding was detected by the addition of 100 ul of 1 ug/ml anti-His-Tag antibody HRP conjugate (R&D Systems, Catalog # MAB050H, clone# AD1.1.10) in binding buffer and incubated at room temperature with shaking at 300 RPM for 1 hour. Wells were washed 3× with wash buffer as above and 100 μL of room temperature Ultra-TMB substrate (Moss Inc.) added. Development was for 7 minutes and the reaction stopped by the addition of 100 μL 2N H2504. The plate was read on Molecular Devices SpectraMax 340PC-384 plate reader set at wavelength of 450 nM.

To assess TIGIT-capture and soluble PVRIG binding of the bispecific antibodies, as similar assay to that used above was performed. In this case, 100 μL/well of 1 μg/ml TIGIT-Fc fusion protein in 1×PBS pH7.4 was coated overnight at 4° C. The coating solution was discarded, and the wells blocked with 1×PBS pH 7.4, 2% BSA and 0.1% Tween 20 for 2 hours at room temperature. Bispecific and monospecific controls used above were serially diluted in the same range as for the PVRIG capture experiment. The remainder of the experiment was the same as above with the exception that PVRIG-His was used for detection of all samples except CPA.9.086 H4 for which TIGIT-His was used.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. An anti-PVRIG/anti-TIGIT bispecific antibody that monovalently binds a human PVRIG and monovalently binds TIGIT for use in activating T cells and/or NK cells for the treatment of cancer.

2. An anti-PVRIG/anti-TIGIT bispecific antibody comprising:
a) a first antigen binding portion comprising:
i. a first heavy chain variable domain comprising a vhCDR1, vhCDR2, and vhCDR3 from an anti-PVRIG antibody; and
ii. a first light chain variable domain comprising a vlCDR1, vlCDR2, and vlCDR3 from an anti-PVRIG antibody;
wherein the anti-PVRIG antibody is selected from the group consisting of CHA.7.518.4, CHA.7.518.1, CHA.7.518, CHA.7.524 CHA.7.530, CHA.7.538_1, CHA.7.538_2, CHA.7.502, CHA.7.503, CHA.7.506, CHA.7.508, CHA.7.510, CHA.7.512, CHA.7.514, CHA.7.516, CHA.7.518, CHA.7.520.1, CHA.7.520.2, CHA.7.522, CHA.7.524, CHA.7.526, CHA.7.527, CHA.7.528, CHA.7.530, CHA.7.534, CHA.7.535, CHA.7.537, CHA.7.538.1, CHA.7.538.2, CHA.7.543, CHA.7.544, CHA.7.545, CHA.7.546, CHA.7.547, CHA.7.548, CHA.7.549, CHA.7.550, CHA7.538.1.2, CPA.7.021, CPA.7.001, CPA.7.003, CPA.7.004, CPA.7.006, CPA.7.008, CPA.7.009, CPA.7.010, CPA.7.011, CPA.7.012, CPA.7.013, CPA.7.014, CPA.7.015, CPA.7.017, CPA.7.018, CPA.7.019, CPA.7.022, CPA.7.023, CPA.7.024, CPA.7.033, CPA.7.034, CPA.7.036, CPA.7.040, CPA.7.046, CPA.7.047, CPA.7.049, CPA.7.050, and CHA.7.518; and
a) a second antigen binding portion comprising an anti-TIGIT antigen binding domain.

3. An anti-PVRIG/anti-TIGIT bispecific antibody comprising:
a) a first antigen binding portion comprising an anti-PVRIG antigen binding domain; and
b) a second antigen binding portion comprising:
i. a second heavy chain variable domain comprising a vhCDR1, vhCDR2, and vhCDR3 from an anti-TIGIT antibody; and
ii. a second light chain variable domain comprising a vlCDR1, vlCDR2, and vlCDR3 from an anti-TIGIT antibody;
wherein the anti-TIGIT antibody is selected from the group consisting of CPA.9.086, CHA.9.547.18, CPA.9.018, CPA.9.027, CPA.9.049, CPA.9.057, CPA.9.059, CPA.9.083, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, and CHA.9.541.8.

4. An anti-PVRIG/anti-TIGIT bispecific antibody comprising:
a) a first antigen binding portion comprising:
i. a first heavy chain variable domain comprising a vhCDR1, vhCDR2, and vhCDR3 from an anti-PVRIG antibody; and
ii. a first light chain variable domain comprising a vlCDR1, vlCDR2, and vlCDR3 from an anti-PVRIG antibody
wherein the anti-PVRIG antibody is selected from the group consisting of CHA.7.518.4, CHA.7.518.1, CHA.7.518, CHA.7.524, CHA.7.530, CHA.7.538_1, CHA.7.538_2, CHA.7.502, CHA.7.503, CHA.7.506, CHA.7.508, CHA.7.510, CHA.7.512, CHA.7.514, CHA.7.516, CHA.7.518, CHA.7.520.1, CHA.7.520.2, CHA.7.522, CHA.7.524, CHA.7.526, CHA.7.527, CHA.7.528, CHA.7.530, CHA.7.534, CHA.7.535, CHA.7.537, CHA.7.538.1, CHA.7.538.2, CHA.7.543, CHA.7.544, CHA.7.545, CHA.7.546, CHA.7.547, CHA.7.548, CHA.7.549, CHA.7.550, CHA7.538.1.2, CPA.7.021, CPA.7.001, CPA.7.003, CPA.7.004, CPA.7.006, CPA.7.008, CPA.7.009, CPA.7.010, CPA.7.011, CPA.7.012, CPA.7.013, CPA.7.014, CPA.7.015, CPA.7.017, CPA.7.018, CPA.7.019, CPA.7.022, CPA.7.023, CPA.7.024, CPA.7.033, CPA.7.034, CPA.7.036, CPA.7.040, CPA.7.046, CPA.7.047, CPA.7.049, and CPA.7.050CHA.7.518; and
b) a second antigen binding portion comprising:
i. a second heavy chain variable domain comprising a vhCDR1, vhCDR2, and vhCDR3 from an anti-TIGIT antibody; and
ii. a second light chain variable domain comprising a vlCDR1, vlCDR2, and vlCDR3 from an anti-TIGIT antibody;
wherein the anti-TIGIT antibody is selected from the group consisting of CPA.9.086, CHA.9.547.18, CPA.9.018, CPA.9.027, CPA.9.049, CPA.9.057, CPA.9.059, CPA.9.083, CPA.9.086, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, and CHA.9.541.8.

5. The anti-PVRIG/anti-TIGIT bispecific antibody of claim 4, wherein first antigen binding portion comprises:
i. a first heavy chain comprising VH-CH1-hinge-CH2-CH3; and
ii. a first light chain comprising VL-CL, wherein the CL is the constant domain of either a kappa or lambda antibody.

6. The anti-PVRIG/anti-TIGIT bispecific antibody of claim 5, wherein the first heavy chain CH3 comprises the amino acid substitutions S354C, E356D, M358L, and T366W.

7. The anti-PVRIG/anti-TIGIT bispecific antibody of claim 5, wherein the CL is kappa.

8. The anti-PVRIG/anti-TIGIT bispecific antibody of claim 4, wherein the second antigen binding portion comprises:
i. a second heavy chain comprising HC-CL-hinge-CH2-CH3, wherein the CL is either kappa or lambda; and
ii. a second light chain comprising VL-CH1.

9. The anti-PVRIG/anti-TIGIT bispecific antibody of claim 8, wherein the second heavy chain CH3 comprises the amino acid substitutions Y349C, E356D, M358L, T366S, L368A, and Y407V.

10. The anti-PVRIG/anti-TIGIT bispecific antibody of claim 8, wherein the CL is lambda.

11. The anti-PVRIG/anti-TIGIT bispecific antibody of claim 8, wherein the CL is kappa.

12. An anti-PVRIG/anti-TIGIT bispecific antibody comprising:
a) a first antigen binding portion comprising an anti-PVRIG antigen binding domain; and
b) a second anti-TIGIT antigen binding portion comprising:
i. a second heavy chain variable region comprising
CPA.9.086 VH
(EVQLVETGGGLIQPGRSLRLSCAASGFTFSSYAMHWVRQAPGKG
LEWVAVISYAGEVKYYADSVKGRFTISRDNSKNTLYLQMNSLRAE
DTAVYYCARDPLPLHYYGMDVWGQGTTVTVSS; SEQ ID
NO: 1634);
and
ii. a second light chain variable region
comprising CPA.9.086 VL
(QSALTQPRSASGNPGQRVTISCSGSSSNMGRRPVNWYQQIPGTAP
KLLIYSQNQRPSGVPDRFSGSQSGTSASLTISGLQSEDEAEYFCAV
WDDIGRVLQLGGGTQLAVL; SEQ ID NO: 1639).

13. An anti-PVRIG/anti-TIGIT bispecific antibody comprising:
c) a first antigen binding portion comprising an anti-PVRIG antigen binding domain; and
d) a second anti-TIGIT antigen binding portion comprising:
i. a second heavy chain variable region comprising
CPA.9.547.18 VH
(EVQLVESGGGLVQPGGSLRLSCAASGFTFSSYIMSWVRQAPGKGL
EWVATISGGSKGQYYADSVKGRFTISRDNSKNTLYLQMNSLRAED
TAVYYCAKWLLSYYAMDYWGQGTLVTVSS; SEQ ID NO: 1664);
and
ii. a second light chain variable region
comprising CPA.9.547.18 VL
(DIQMTQSPSSLSASVGDRITITCRASQSMAIWLSWYQQKPGKAPK
LLIYKASKSHTGVPSRFSGSGSGTDFTLTISSLQPEDFATYYCQQGQ
SYPYTFGQGTKLEIK; SEQ ID NO: 1668).

14. An anti-PVRIG/anti-TIGIT bispecific antibody comprising:
a) a first antigen binding portion comprising an anti-PVRIG binding doming comprising:
i. a first heavy chain variable region comprising
CHA.7.518.1 VH
(QVQLVQSGAEVKKPGASVKVSCKASGYTFTDYNINWVRQAPGQ
GLEWMGYIYPYIGGSGYAQKFQGRVTMTRDTSTSTVYMELSSLRS
EDTAVYYCAREDKTARNAMDYWGQGTLVTVSS; SEQ ID
NO: 1539);
and
ii. a first light chain comprising CHA.7.518.1 VL
(DIQMTQSPSSLSASVGDRVTITCRVSENIYSNLAWYQQKPGKAPK
LLIYEATNLAEGVPSRFSGSGSGTDFTLTISSLQPEDFATYYCQHFW
GTPYTFGQGTKLEIK; SEQ ID NO: 1544);
and
b) a second antigen binding portion comprising an anti-TIGIT antigen binding domain.

15. An anti-PVRIG/anti-TIGIT bispecific antibody comprising:
a) a first antigen binding portion comprising an anti-PVRIG binding doming comprising:
i. a first heavy chain variable region comprising
CHA.7.518.4 VH
(QVQLVQSGAEVKKPGASVKVSCKASGYTFTDYNINWVRQAPGQ
GLEWMGYIYPYIGGSGYAQKFQGRVTMTRDTSTSTVYMELSSLRS
EDTAVYYCAREDKTARNAMDYWGQGTLVTVSS; SEQ ID
NO: 3179);
and
ii. a first light chain comprising CHA.7.518.4 VL
(DIQMTQSPSSLSASVGDRVTITCRVSENIYDVLAWYQQKPGKAPK
LLIYEATNLAEGVPSRFSGSGSGTDFTLTISSLQPEDFATYYCQHFW
GTPYTFGQGTKLEIK; SEQ ID NO: 3180);
and
b) a second antigen binding portion comprising an anti-TIGIT antigen binding domain.

16. An anti-PVRIG/anti-TIGIT bispecific antibody comprising:
a) a first antigen binding portion comprising an anti-PVRIG binding doming comprising:
i. a first heavy chain variable region comprising
CHA.7.518.1 VH
(QVQLVQSGAEVKKPGASVKVSCKASGYTFTDYNINWVRQAPGQ
GLEWMGYIYPYIGGSGYAQKFQGRVTMTRDTSTSTVYMELSSLRS
EDTAVYYCAREDKTARNAMDYWGQGTLVTVSS; SEQ ID
NO: 1539);
and
ii. a first light chain variable region comprising
CHA.7.518.1
(DIQMTQSPSSLSASVGDRVTITCRVSENIYSNLAWYQQKPGKAPK
LLIYEATNLAEGVPSRFSGSGSGTDFTLTISSLQPEDFATYYCQHFW
GTPYTFGQGTKLEIK; SEQ ID NO: 1544);
and
b) a second antigen binding portion comprising an anti-TIGIT binding doming comprising
i. a first heavy chain variable region comprising
CPA.9.086 VH
(EVQLVETGGGLIQPGRSLRLSCAASGFTFSSYAMHWVRQAPGKG
LEWVAVISYAGEVKYYADSVKGRFTISRDNSKNTLYLQMNSLRAE
DTAVYYCARDPLPLHYYGMDVWGQGTTVTVSS; SEQ ID
NO: 1634);
and
ii. a first light chain variable region comprising
CPA.9.086 VL
(QSALTQPRSASGNPGQRVTISCSGSSSNMGRRPVNWYQQIPGTAP
KLLIYSQNQRPSGVPDRFSGSQSGTSASLTISGLQSEDEAEYFCAV
WDDIGRVLQLGGGTQLAVL; SEQ ID NO: 1639).

17. An anti-PVRIG/anti-TIGIT bispecific antibody comprising:
a) a first antigen binding portion comprising an anti-PVRIG binding doming comprising:
i. a first heavy chain variable region comprising
CHA.7.518.4 VH
(QVQLVQSGAEVKKPGASVKVSCKASGYTFTDYNINWVRQAPGQ
GLEWMGYIYPYIGGSGYAQKFQGRVTMTRDTSTSTVYMELSSLRS
EDTAVYYCAREDKTARNAMDYWGQGTLVTVSS; SEQ ID
NO: 3179);
and
ii. a first light chain comprising CHA.7.518.4 VL
(DIQMTQSPSSLSASVGDRVTITCRVSENIYDVLAWYQQKPGKAPK
LLIYEATNLAEGVPSRFSGSGSGTDFTLTISSLQPEDFATYYCQHFW
GTPYTFGQGTKLEIK; SEQ ID NO: 3180);
and
b) a second antigen binding portion comprising an anti-TIGIT binding doming comprising:
i. a first heavy chain variable region comprising
CPA.9.086 VH
(EVQLVETGGGLIQPGRSLRLSCAASGFTFSSYAMHWVRQAPGKG
LEWVAVISYAGEVKYYADSVKGRFTISRDNSKNTLYLQMNSLRAE
DTAVYYCARDPLPLHYYGMDVWGQGTTVTVSS; SEQ ID
NO: 1634);
and
ii. a first light chain variable region comprising
CPA.9.086 VL
(QSALTQPRSASGNPGQRVTISCSGSSSNMGRRPVNWYQQIPGTAP
KLLIYSQNQRPSGVPDRFSGSQSGTSASLTISGLQSEDEAEYFCAV
WDDIGRVLQLGGGTQLAVL; SEQ ID NO: 1639).

18. An anti-PVRIG/anti-TIGIT bispecific antibody comprising:
a) a first antigen binding portion comprising an anti-PVRIG binding doming comprising:
i. a first heavy chain variable region comprising
CHA.7.518.1
(QVQLVQSGAEVKKPGASVKVSCKASGYTFTDYNINWVRQAPGQ
GLEWMGYIYPYIGGSGYAQKFQGRVTMTRDTSTSTVYMELSSLRS
EDTAVYYCAREDKTARNAMDYWGQGTLVTVSS; SEQ ID NO:
1539);
and
ii. a first light chain variable region comprising
CHA.7.518.1 VL
(DIQMTQSPSSLSASVGDRVTITCRVSENIYSNLAWYQQKPGKAPK
LLIYEATNLAEGVPSRFSGSGSGTDFTLTISSLQPEDFATYYCQHFW
GTPYTFGQGTKLEIK; SEQ ID NO: 1544);
and
b) a second antigen binding portion comprising an anti-TIGIT binding doming comprising:
i. a first heavy chain variable region comprising
CHA.9.547.18 HC
(EVQLVESGGGLVQPGGSLRLSCAASGFTFSSYIMSWVRQAPGKGL
EWVATISGGSKGQYYADSVKGRFTISRDNSKNTLYLQMNSLRAED
TAVYYCAKWLLSYYAMDYWGQGTLVTVSS; SEQ ID NO: 1664);
and
ii. a first light chain variable region comprising
CHA.9.547.18 VL
(DIQMTQSPSSLSASVGDRITITCRASQSMAIWLSWYQQKPGKAPK
LLIYKASKSHTGVPSRFSGSGSGTDFTLTISSLQPEDFATYYCQQGQ
SYPYTFGQGTKLEIK; SEQ ID NO: 1668).

19. An anti-PVRIG/anti-TIGIT bispecific antibody comprising:
a) a first antigen binding portion comprising an anti-PVRIG binding doming comprising:
i. a first heavy chain variable region comprising
CHA.7.518.4 VH
(QVQLVQSGAEVKKPGASVKVSCKASGYTFTDYNMWVRQAPGQ
GLEWMGYIYPYIGGSGYAQKFQGRVTMTRDTSTSTVYMELSSLRS
EDTAVYYCAREDKTARNAMDYWGQGTLVTVSS; SEQ ID
NO: 3179);
and
ii. a first light chain comprising CHA.7.518.4 VL
(DIQMTQSPSSLSASVGDRVTITCRVSENIYDVLAWYQQKPGKAPK
LLIYEATNLAEGVPSRFSGSGSGTDFTLTISSLQPEDFATYYCQHFW
GTPYTFGQGTKLEIK; SEQ ID NO: 3180);
and
b) a second antigen binding portion comprising an anti-TIGIT binding doming comprising:
i. a first heavy chain variable region comprising
CHA.9.547.18 HC
(EVQLVESGGGLVQPGGSLRLSCAASGFTFSSYIMSWVRQAPGKGL
EWVATISGGSKGQYYADSVKGRFTISRDNSKNTLYLQMNSLRAED
TAVYYCAKWLLSYYAMDYWGQGTLVTVSS (SEQ ID NO: 1664);
and
ii. a first light chain variable region comprising
CHA.9.547.18 VL
(DIQMTQSPSSLSASVGDRITITCRASQSMAIWLSWYQQKPGKAPK
LLIYKASKSHTGVPSRFSGSGSGTDFTLTISSLQPEDFATYYCQQGQ
SYPYTFGQGTKLEIK; SEQ ID NO: 1668).

20. An anti-PVRIG/anti-TIGIT bispecific antibody according to claim 1 wherein said anti-PVRIG/anti-TIGIT bispecific antibody is a humanized antibody.

21. A composition comprising an anti-PVRIG/anti-TIGIT bispecific antibody according to claim 1.

22. A nucleic acid composition comprising:
a) a first nucleic acid encoding a first heavy chain or heavy chain variable domain according to claim 1;
b) a second nucleic acid encoding a first light chain or light chain variable domain according to claim 1;
c) a third nucleic acid encoding a second heavy chain or heavy chain variable domain according to claim 1; and
d) a fourth nucleic acid encoding a second light chain or light chain variable domain according to claim 1.

23. An expression vector composition comprising:
a) a first expression vector comprising the first nucleic acid of claim 22;
b) a second expression vector comprising the second nucleic acid of claim 22;
c) a third expression vector comprising the third nucleic acid of claim 22; and
d) a fourth expression vector comprising the fourth nucleic acid of claim 22.

24. (canceled)

25. A host cell comprising the expression vector composition of claim 23.

26. A method of making an anti-PVRIG/anti-TIGIT bispecific antibody comprising:
a) culturing the host cell of claim 25 under conditions wherein the antibody is expressed; and
b) recovering the antibody.

27. A method of activating T cells of a patient comprising administering the anti-PVRIG/anti-TIGIT bispecific antibody of claim 1 to the patient, wherein a subset of the T cells of the patient are activated.

28. A method of activating cytotoxic T cells (CTLs) of a patient comprising administering the anti-PVRIG/anti-TIGIT bispecific antibody of claim 1 to the patient, wherein a subset of the CTLs of the patient are activated.

29. A method of activating NK cells of a patient comprising administering the anti-PVRIG/anti-TIGIT bispecific antibody of claim 1 to the patient, wherein a subset of the NK cells of the patient are activated.

30. A method of activating γδ T cells of a patient comprising administering the anti-PVRIG/anti-TIGIT bispecific antibody of claim 1 to the patient, wherein a subset of the γδ T cells of the patient are activated.

31. A method of activating Th1 cells of a patient comprising administering the anti-PVRIG/anti-TIGIT bispecific antibody of claim 1 to the patient, wherein a subset of the Th1 cells of the patient are activated.

32. A method of decreasing or eliminating cell number and/or activity of at least one of regulatory T cells (Tregs) in a patient comprising administering the anti-PVRIG/anti-TIGIT bispecific antibody of claim 1 to the patient.

33. A method of increasing interferon-γ production and/or pro-inflammatory cytokine secretion in a patient comprising administering the anti-PVRIG/anti-TIGIT bispecific antibody of claim 1 to the patient.

34. A method of treating cancer in a patient comprising administering the anti-PVRIG/anti-TIGIT antibody of claim 1 to the patient.

35. (canceled)

36. (canceled)

37. An anti-PVRIG/anti-TIGIT bispecific antibody comprising:
a) a first antigen binding portion comprising an anti-PVRIG antigen binding portion comprising:
i. a first heavy chain variable domain comprising a vhCDR1, vhCDR2, and vhCDR3 from an anti-PVRIG antibody; and
ii. a first light chain variable domain comprising a vlCDR1, vlCDR2, and vlCDR3 from an anti-PVRIG antibody;
wherein the anti-PVRIG antigen binding portion is selected from the group consisting of CPA.7.021, CPA.7.001, CPA.7.003, CPA.7.004, CPA.7.006, CPA.7.008, CPA.7.009, CPA.7.010, CPA.7.011, CPA.7.012, CPA.7.013, CPA.7.014, CPA.7.015, CPA.7.017, CPA.7.018, CPA.7.019, CPA.7.022, CPA.7.023, CPA.7.024, CPA.7.033, CPA.7.034, CPA.7.036, CPA.7.040, CPA.7.046, CPA.7.047, CPA.7.049, CPA.7.050, CHA.7.502, CHA.7.503, CHA.7.506, CHA.7.508, CHA.7.510, CHA.7.512, CHA.7.514, CHA.7.516, CHA.7.518, CHA.7.520.1, CHA.7.520.2, CHA.7.522, CHA.7.524, CHA.7.526, CHA.7.527, CHA.7.528, CHA.7.530, CHA.7.534, CHA.7.535, CHA.7.537, CHA.7.538.1, CHA.7.538.2, CHA.7.543, CHA.7.544, CHA.7.545, CHA.7.546, CHA.7.547, CHA.7.548, CHA.7.549, CHA.7.550, CHA.7.518.1; CHA7.538.1.2 and CHA.7.518.4; and
b) a second antigen binding portion comprising an anti-TIGIT antigen binding domain, wherein the anti-TIGIT antigen binding domain is from an antibody as provided in FIGS. 24 and 41, and in particular FIG. 24A-24EE.

38. An anti-PVRIG/anti-TIGIT bispecific antibody comprising:
a) a first antigen binding portion comprising an anti-PVRIG antigen binding domain wherein the anti-PVRIG antigen binding domain is from an antibody as provided in FIG. 35; and
b) a second antigen binding portion comprising an anti-TIGIT antigen binding domain comprising:
i. a second heavy chain variable domain comprising a vhCDR1, vhCDR2, and vhCDR3 from an anti-TIGIT antibody; and
ii. a second light chain variable domain comprising a vlCDR1, vlCDR2, and vlCDR3 from an anti-TIGIT antibody;
wherein the anti-TIGIT antigen binding domain is selected from the group consisting of CPA.9.018, CPA.9.027, CPA.9.049, CPA.9.057, CPA.9.059, CPA.9.083, CPA.9.086, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, CHA.9.541.8, and CHA.9.547.18.

39. An anti-PVRIG antibody comprising:
i) a heavy chain or heavy chain variable domain
comprising the vhCDR1, vhCDR2, and vhCDR3
from the following sequence:
(SEQ ID NO: 3179)
QVQLVQSGAEVKKPGASVKVSCKASGYTFTDYNINWVRQAPGQGLEWMGY
IYPYIGGSGYAQKFQGRVTMTRDTSTSTVYMELSSLRSEDTAVYYCARED
KTARNAMDYWGQGTLVTVSS,
and
ii) a light chain or light chain variable domain
comprising the vlCDR1, vlCDR2, vlCDR3 from
the following sequence:
(SEQ ID NO: 3180)
DIQMTQSPSSLSASVGDRVTITCRVSENIYDVLAWYQQKPGKAPKLLIYE
ATNLAEGVPSRFSGSGSGTDFTLTISSLQPEDFATYYCQHFWGTPYTF
GQGTKLEIK.

40. An anti-TIGIT antibody comprising:
i) a heavy chain or heavy chain variable domain
comprising the vhCDR1, vhCDR2, and vhCDR3 from
the following sequence:
(SEQ ID NO: 1664)
EVQLVESGGGLVQPGGSLRLSCAASGFTFSSYIMSWVRQAPGKGLEWVA
TISGGSKGQYYADSVKGRFTISRDNSKNTLYLQMNSLRAEDTAVYYCAKW
LLSYYAMDYWGQGTLVTVSS,
and
ii) a light chain or light chain variable domain
comprising the vlCDR1, vlCDR2, and vlCDR3
from the following sequence:
(SEQ ID NO: 1668)
DIQMTQSPSSLSASVGDRITITCRASQSMAIWLSWYQQKPGKAPKLLIYK
ASKSHTGVPSRFSGSGSGTDFTLTISSLQPEDFATYYCQQGQSYPYTFG
QGTKLEIK.

41. (canceled)

42. A composition comprising an anti-PVRIG antibody according to claim 38.

43. A nucleic acid composition comprising:
i) a first nucleic acid encoding a heavy chain or heavy chain variable domain according to claim 38; and
ii) a second nucleic acid encoding a light chain or light chain variable domain according to claim 38.

44. (canceled)

45. A nucleic acid composition comprising:
i) a first nucleic acid encoding a heavy chain or heavy chain variable domain according to claim 38; and
ii) a second nucleic acid encoding a light chain or light chain variable domain according to claim 38.

46. (canceled)

47. (canceled)

48. An expression vector composition comprising:
i) a first expression vector comprising the first nucleic acid of claim 43; and
ii) a second expression vector comprising the second nucleic acid of claim 43.

49. An expression vector comprising:
i) the first nucleic acid of claim 43; and
ii) the second nucleic acid of claim 43.

50. A host cell comprising the expression vector or vector composition of anyone of claim 48.

51. A method of making an anti-PVRIG or anti-TIGIT antibody comprising:
i) culturing the host cell of claim 48 under conditions wherein the antibody is expressed; and
ii) recovering the antibody.

52.-58. (canceled)

59. A method of treating cancer in a patient comprising administering the anti-PVRIG or anti-TIGIT antibody of claim 38 to the patient.

60. (canceled)

61. (canceled)

62. A method of treating cancer in a patient comprising administering a combination therapy comprising an anti-PVRIG/anti-TIGIT bispecific antibody according to claim 1.

63. A method of treating cancer in a patient comprising administering a combination therapy comprising an anti-PVRIG antibody of claim 38 and an anti-PD-1 antibody.

64. (canceled)

65. A method of treating cancer in a patient comprising administering a combination therapy comprising an anti-TIGIT antibody of claim 39 and an anti-PD-1 antibody.

66. (canceled)

67. A method of treating cancer in a patient comprising administering a combination therapy comprising an anti-TIGIT antibody of claim 39 and an anti-PVRIG antibody of claim 38.

68. A method of treating cancer in a patient comprising administering a triple combination therapy comprising an anti-TIGIT antibody of claim 39, an anti-PVRIG antibody, and an anti-PD-1 antibody.

69. A method of treating cancer in a patient comprising administering a triple combination therapy comprising an anti-TIGIT, an anti-PVRIG antibody of claim 38, and an anti-PD-1 antibody.

70. A method of treating cancer in a patient comprising administering a triple combination therapy comprising an anti-TIGIT of claim 39, an anti-PVRIG antibody of claim 38, and an anti-PD-1 antibody.

71.-80. (canceled)
